(19) United States
(12) Patent Application Publication
(10) Pub. No.: US 2024/0279237 A1
(43) Pub. Date: Aug. 22, 2024

(54) FUSED BICYCLIC HETEROARYL COMPOUNDS USEFUL AS NLRP3 INHIBITORS

(71) Applicant: Hoffmann-La Roche Inc., Little Falls, NJ (US)

(72) Inventors: Lewis Scott AITKEN, Nottingham (GB); Lea Aurelie BOUCHE, Basel (CH); Wolfgang GUBA, Muellheim (DE); Georg JAESCHKE, Basel (CH); Heather Jennifer JOHNSTON, Nottingham (GB); Stefanie Katharina MESCH, Basel (CH); Angélique PATINY-ADAM, Krembs (FR); Christian SCHNIDER, Biel-Benken, Basel-Country (CH); Jonathan Martin SHANNON, Nottingham (GB); Sandra STEINER, Sursee, Lucerne (CH); Andreas TOSSTORFF, New York, NY (US)

(73) Assignee: Hoffmann-La Roche Inc., Little Falls, NJ (US)

(21) Appl. No.: 18/637,966

(22) Filed: Apr. 17, 2024

Related U.S. Application Data
(63) Continuation of application No. PCT/EP2022/078755, filed on Oct. 17, 2022.

(30) Foreign Application Priority Data
Oct. 19, 2021 (EP) 21203314.6
May 23, 2022 (EP) 22174872.6

(51) Int. Cl. C07D 498/04; A61K 9/20; A61K 9/48; A61K 31/437; A61K 31/4375; A61K 31/438; A61K 31/439; A61K 31/4545; A61K 31/496; A61K 31/4985; A61K 31/5377; A61K 31/5383; C07D 471/04; C07D 519/00

(52) U.S. Cl. CPC C07D 498/04; A61K 9/2009; A61K 9/2054; A61K 9/2059; A61K 9/485; A61K 9/4858; A61K 9/4866; A61K 31/437; A61K 31/4375; A61K 31/438; A61K 31/439; A61K 31/4545; A61K 31/496; A61K 31/4985; A61K 31/5377; A61K 31/5383; C07D 471/04; C07D 519/00

(57) ABSTRACT

The invention relates to novel compounds having the general formula Ic

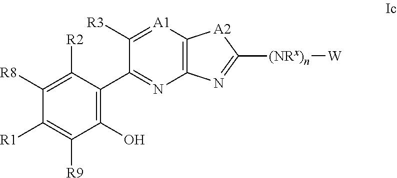

wherein R¹, R², R³, R⁸, R⁹, R^{X}, A¹, A², W and n are as described herein, composition including the compounds and methods of using the compounds.

### FIELD OF THE INVENTION

The present invention relates to organic compounds useful for therapy and/or prophylaxis in a mammal, and in particular to compounds that modulate NLRP3 inhibition.

The present invention provides novel compounds of formula Ic

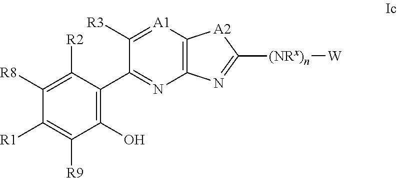

wherein
A¹ is —N— or —CR¹⁰—;
A² is —O—, —NH—, —NCH₃—, or —S—;
R¹ is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 4-to-6-membered cycloalkyl or heterocycle comprising a single O heteroatom;
R² is H, halo, alkyl, cyano, haloalkoxy, haloalkyl, alkoxy, alkoxyalkyl, or hydroxyalkyl, and R³ is H, halo, or alkyl, wherein at least one of R² and R³ is not H;
R⁸ is H or halo;
R¹⁰ is selected from H, alkyl, acetyl, alkylamino, dialkylamino, hydroxyalkyl, hydroxyalkylalkyl, cyano, halo, haloalkyl, alkoxy, haloalkoxy, and NR′R″, wherein R′ and R″ are independently selected from H and alkyl, or R′ and R″ and the N atom to which they are attached form either a 4-to-6 membered N-containing heterocycle optionally substituted with —OH, or a 5-membered lactam ring;
n can be 0 or 1;
R^{X} is H, alkyl, alkoxyalkyl, or hydroxyalkyl;
W is selected from
   a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, cyano, haloalkyl, alkylester, alkylsulfon, cycloalkyl, heterocycle comprising a single O heteroatom, cycloalkyl substituted with —OH, cycloalkyl-CH₂— substituted with —OH, a 6-membered heteroaryl substituted with alkoxy, and a 5-membered heteroaryl with 2 N heteroatoms,
   a 4-to-6-membered heterocycle-CH₂— comprising up to 2 heteroatoms independently selected from N and O, wherein the maximum number of O heteroatoms is 1, optionally substituted by alkyl,
   a 3-to-6 membered cycloalkyl optionally substituted with 1 to 2 substituents independently selected from —OH, alkylamino and dialkylamino,
   a 4-membered cycloalkyl-CH₂— optionally substituted with 1 or 2 substituents independently selected from —OH and alkyl, and
   a 5-membered heteroaryl-CH₂— comprising 2 N heteroatoms;
and pharmaceutically acceptable salts.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β B signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula Ic

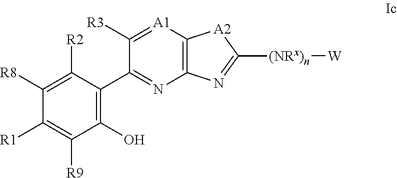

wherein
A¹ is —N— or —CR¹⁰—;
A² is —O—, —NH—, —NCH₃—, or —S—;
R¹ is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 4-to-6-membered cycloalkyl or heterocycle comprising a single O heteroatom;
R² is H, halo, alkyl, cyano, haloalkoxy, haloalkyl, alkoxy, alkoxyalkyl, or hydroxyalkyl, and R³ is H, halo, or alkyl, wherein at least one of R² and R³ is not H;
R⁸ is H or halo;
R¹⁰ is selected from H, alkyl, acetyl, alkylamino, dialkylamino, hydroxyalkyl, hydroxyalkylalkyl, cyano, halo, haloalkyl, alkoxy, haloalkoxy, and NR′R″, wherein R′ and R″ are independently selected from H and alkyl, or R′ and R″ and the N atom to which they are attached form either a 4-to-6 membered N-containing heterocycle optionally substituted with —OH, or a 5-membered lactam ring;
n can be 0 or 1;
R^{X} is H, alkyl, alkoxyalkyl, or hydroxyalkyl;
W is selected from
   a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, cyano, haloalkyl, alkylester, alkylsulfon, cycloalkyl, heterocycle comprising a single O heteroatom, cycloalkyl substituted with —OH, cycloalkyl-CH₂— substituted with —OH, a 6-membered heteroaryl substituted with alkoxy, and a 5-membered heteroaryl with 2 N heteroatoms,
   a 4-to-6-membered heterocycle-CH₂— comprising up to 2 heteroatoms independently selected from N and O, wherein the maximum number of O heteroatoms is 1, optionally substituted by alkyl,
   a 3-to-6 membered cycloalkyl optionally substituted with 1 to 2 substituents independently selected from —OH, alkylamino and dialkylamino,
   a 4-membered cycloalkyl-CH₂— optionally substituted with 1 or 2 substituents independently selected from —OH and alkyl, and
   a 5-membered heteroaryl-CH₂— comprising 2 N heteroatoms;
and pharmaceutically acceptable salts.

The term “acetyl” denotes an —C(═O)CH₃ group.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl.

The term “alkoxy” denotes a group of the formula —O—R′, wherein R′ is a C₁₋₆-alkyl group.

Examples of C₁₋₆-alkoxy groups include methoxy, ethoxy, n-propoxy, isopropoxy, n-butoxy, isobutoxy and tert-butoxy. Particular alkoxy groups include methoxy and ethoxy.

The term “alkoxyalkyl” denotes an alkyl group wherein one of the hydrogen atoms of the alkyl group have been replaced by an alkoxy group. Particular examples of alkoxyalkyl are methoxymethyl and methoxyethyl.

The term “amino” denotes an —NH₂ group.

The term “alkylamino” denotes an amino group wherein one of the hydrogen atoms of the amino group have been replaced by an alkyl group. Particular example is methylamino.

The term “alkylaminoalkyl” denotes an aminoalkyl group wherein one of the hydrogen atoms of the amino group has been replaced by an alkyl group. Examples of alkylaminoalkyl groups include methylaminomethyl and methylaminoethyl.

The term “dialkylamino” denotes an amino group wherein two of the hydrogen atoms of the amino group have been replaced by two alkyl groups. Particular example is dimethylamino.

The term “dialkylaminoalkyl” denotes an alkyl group wherein at least one of the hydrogen atoms of the alkyl group has been replaced by a dialkylamino group. Examples of dialkylaminoalkyl include (dimethylamino)methyl and (dimethylamino)ethyl. Particular example is (dimethylamino)methyl.

The term “aminoalkyl” denotes an alkyl group wherein at least one of the hydrogen atoms of the alkyl group has been replaced by an amino group. Examples of aminoalkyl include aminomethyl and aminoethyl.

The term “ester” denotes a carboxyl group bridging two moieties linked at carbon atoms. Examples include methoxycarbonyl.

The term “alkylester” denotes an ester group wherein one of the hydrogen atoms of the ester group have been replaced by an alkyl group. Particular examples are 2-methoxy-2-oxo-ethyl and 3-methoxy-3-oxo-propyl.

The term “sulfonyl” denotes an —S(O)₂— group.

The term “alkylsulfonyl” denotes a group of the formula —S(O)2-R′, wherein R′ is an alkyl group. Particular examples of alkylsulfonyl groups include groups of the formula —S(O)2-R′, wherein R′ is methyl.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl, cyclohexyl, octahydropentalenyl, spiro[3.3]heptanyl, and the like. Particular examples include cyclobutyl, cyclopentyl, and cyclohexyl. Other particular examples include cyclopropyl and cyclohexyl.

The term “cyano” denotes a —C—N group.

The term “halogen”, “halide” and “halo” are used interchangeably herein and denote fluoro, chloro, bromo or iodo. Particular halogens are fluoro and chloro.

The term “haloalkyl” denotes a C₁₋₆-alkyl group wherein at least one of the hydrogen atoms of the C₁₋₆-alkyl group has been replaced by the same or different halogen atoms. Example of haloalkyl include fluoromethyl, difluoromethyl and trifluoromethyl. Particular examples are difluoromethyl, difluoropropyl and trifluoromethyl.

The term “haloalkoxy” denotes a C₁₋₆-alkoxy group wherein at least one of the hydrogen atoms of the C₁₋₆-alkoxy group has been replaced by the same or different halogen atoms. Examples of haloalkoxy are difluoromethoxy, trifluoromethoxy, difluoroethoxy and trifluoroethoxy. Particular examples are difluoromethoxy and trifluoromethoxy.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl group include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridinyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, and benzothiophenyl. A particular example of a heteroaryl group is imidazole

The term “heterocycle ring” or “heterocycle” denotes a monovalent saturated or partly unsaturated mono- or bicycle ring system of 4 to 10 ring atoms, or 4 to 9 ring atoms, comprising 1, 2, or 3 ring heteroatoms selected from N, O and S, the remaining ring atoms being carbon. Examples for monocyclic saturated heterocycle rings are azetidinyl, diazepanyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, morpholinyl, and piperazinyl. Examples of polycyclic saturated heterocycle rings are azaspiroheptanyl, diazaspiroheptanyl, azaspirooctanyl, diazospirooctanyl, diazaspirononanyl, oxaazaspirooctanyl, and oxadiazaspirononanyl. Particular examples of 4-member heterocycle rings are azetidinyl and oxetanyl. Particular examples of 5-member heterocycle rings are pyrrolidinyl, tetrahydrofuranyl, and pyrrolidinyl. Particular examples of 6-member heterocycle rings are piperidyl, morpholinyl, tetrahydropyranyl, and piperazinyl. A particular example of a 7-member heterocycle ring is azepanyl. Particular examples of 8-member heterocycle rings are azabicyclo[2.2.2]octan-4-yl and Hexahydrofuro[3,4-c]pyrrol-5-yl. Particular examples of 9-member heterocycle rings are octahydroindolizinyl, octahydropyrrolo[2,3-c]pyridin-1-yl, diazaspirononan7-yl, and hexahydro-2H-pyrrolo[3,4-b][1,4]oxazin-4-yl. Particular examples of 10-member heterocycle rings are octahydro-1,7-naphthyridin-1-yl and hexahydro-2H-pyrido[4,3-b][1,4]oxazin-4-yl.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxypropyl, hydroxymethylethyl, hydroxymethylpropyl and dihydroxypropyl. Particular examples are hydroxymethyl, hydroxyethyl, hydroxypropyl and hydroxymethylethyl.

The term “lactam ring” denotes a cyclic amide. Particular example is 2-oxopyrrolidin-1-yl.

The term “oxo” denotes a divalent oxygen atom ═O.

The term “pyrrolidinylalkyl” denotes an alkyl group wherein at least one of the hydrogen atoms of the alkyl group has been replaced by a pyrrolidinyl group. Example of pyrrolidinylalkyl is pyrrolidinylmethyl.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula Ic can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula Ic as described herein and pharmaceutically acceptable salts or esters thereof, in particular compounds according to formula Ic as described herein and pharmaceutically acceptable salts thereof, more particularly compounds according to formula Ic as described herein.

An embodiment of the present invention provides compounds according to formula Ic as described herein, wherein A² is —O— or —NH—.

An embodiment of the present invention provides compounds according to formula Ic as described herein, wherein A² is —O—.

An embodiment of the present invention provides compounds according to formula Ic as described herein, wherein
R¹ is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 5-membered heterocycle comprising a single O heteroatom.

An embodiment of the present invention provides compounds according to formula Ic as described herein, wherein
R¹ is halo, alkyl, haloalkyl, or cyano;
R⁹ is H;
or R¹ and R⁹ form a 5-membered heterocycle comprising a single O heteroatom.

An embodiment of the present invention provides compounds according to formula Ic as described herein, wherein R¹ is halo or cyano and R⁹ is H.

An embodiment of the present invention provides compounds according to formula Ic as described herein, wherein R² is alkyl or alkoxyalkyl and R³ is H.

An embodiment of the present invention provides compounds according to formula Ic as described herein, wherein R⁸ is H.

An embodiment of the present invention provides compounds according to formula Ic as described herein, wherein R¹⁰ is selected from H, alkyl, acetyl, alkylamino, dialkylamino, hydroxyalkyl, hydroxyalkylalkyl, cyano, alkoxy, and NR′R″, wherein R′ and R″ and the N atom to which they are attached form either a 4-to-5-membered N-containing heterocycle optionally substituted with —OH, or a 5-membered lactam ring.

An embodiment of the present invention provides compounds according to formula Ic as described herein, wherein R¹⁰ is selected from H, cyano, and alkoxy.

An embodiment of the present invention provides compounds according to formula Ic as described herein, wherein R¹⁰ is H.

An embodiment of the present invention provides compounds according to formula Ic as described herein, wherein R^{X} is H, alkoxyalkyl, or hydroxyalkyl.

An embodiment of the present invention provides compounds according to formula Ic as described herein, wherein R^{X} is H.

An embodiment of the present invention provides compounds according to formula Ic as described herein, wherein W is selected from
a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, haloalkyl, a 4-membered cycloalkyl, a 4-membered heterocycle comprising 1 O heteroatom, a 4-membered cycloalkyl substituted with —OH, and a 4-membered cycloalkyl-CH₂— substituted with —OH,
a 4-to-6-membered heterocycle-CH₂— comprising up to 2 heteroatoms independently selected from N and O, wherein the maximum number of O heteroatoms is 1, optionally substituted by alkyl,
a 3-to-6-membered cycloalkyl optionally substituted with —OH, alkylamino or dialkylamino,
a 4-membered cycloalkyl-CH₂— optionally substituted with 1 or 2 substituents independently selected from —OH and alkyl, and
a 5-membered heteroaryl-CH₂— comprising 2 N heteroatoms.

An embodiment of the present invention provides compounds according to formula Ic as described herein, wherein W is selected from
a 6-to-9-membered heterocycle comprising up to 3 heteroatoms independently selected from N and O, wherein the maximum number of O heteroatoms is 1, optionally substituted by 1 or 2 substituents selected from oxo, —OH, halo, alkyl and hydroxyalkyl,
a 6-membered heterocycle-CH₂— comprising a single N heteroatom, substituted by alkyl, and
a 5-membered cycloalkyl substituted with dialkylamino.

An embodiment of the present invention provides compounds according to formula Ic as described herein, wherein W is a 6-membered heterocycle comprising a single N heteroatom, optionally substituted by either alkyl or oxo, or both alkyl and —OH; An embodiment of the present invention provides compounds according to formula Ic as described herein, wherein
A¹ is —N— or —CR¹⁰—;
A² is —O— or —NH—;
R¹ is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 5-membered heterocycle comprising a single O heteroatom;
R² is H, halo, alkyl, cyano, haloalkoxy, haloalkyl, alkoxy, alkoxyalkyl, or hydroxyalkyl, and R³ is H, halo, or alkyl, wherein at least one of R² and R³ is not H;
R⁸ is H or halo;
R¹⁰ is selected from H, alkyl, acetylalkylamino, dialkylamino, hydroxyalkyl, hydroxyalkylalkyl, cyano, alkoxy, and NR′R″, wherein R′ and R″ and the N atom to which they are attached form either a 4-to-5-membered N-containing heterocycle optionally substituted with —OH, or a 5-membered lactam ring;
n can be 0 or 1;
R^{X} is H, alkoxyalkyl, or hydroxyalkyl;
W is selected from
   a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, haloalkyl, a 4-membered cycloalkyl, a 4-membered heterocycle comprising a single O heteroatom, a 4-membered cycloalkyl substituted with —OH, and a 4-membered cycloalkyl-CH₂— substituted with —OH,
   a 4-to-6-membered heterocycle-CH₂— comprising up to 2 heteroatoms independently selected from N and O, wherein the maximum number of O heteroatoms is 1, optionally substituted by alkyl,
   a 3-to-6-membered cycloalkyl optionally substituted with —OH, alkylamino or dialkylamino,
   a 4-membered cycloalkyl-CH₂— optionally substituted with 1 or 2 substituents independently selected from —OH and alkyl, and
   a 5-membered heteroaryl-CH₂— comprising 2 N heteroatoms;
and pharmaceutically acceptable salts.

An embodiment of the present invention provides compounds according to formula Ic as described herein, wherein
A¹ is —N— or —CR¹⁰—;
A² is —O—;
R¹ is halo, alkyl, haloalkyl, or cyano;
R⁹ is H;
or R¹ and R⁹ form a 5-membered heterocycle comprising a single O heteroatom;
R² is alkyl or alkoxyalkyl and R³ is H;
R⁸ is H;
R¹⁰ is selected from H, cyano and alkoxy;
n can be 0 or 1;
R^{X} is H;
W is selected from
   a 6-to-9-membered heterocycle comprising up to 3 heteroatoms independently selected from N and O, wherein the maximum number of O heteroatoms is 1, optionally substituted by 1 or 2 substituents selected from oxo, —OH, halo, alkyl and hydroxyalkyl,
   a 6-membered heterocycle-CH₂— comprising a single N heteroatom, substituted by alkyl, and
   a 5-membered cycloalkyl substituted with dialkylamino;
and pharmaceutically acceptable salts.

An embodiment of the present invention provides compounds according to formula Ic as described herein, wherein
A¹ is —N— or —CR¹⁰—;
A² is —O—;
R¹ is halo or cyano;
R⁹ is H;
R² is alkyl or alkoxyalkyl and R³ is H;
R⁸ is H;
R¹⁰ is H;
n can be 0 or 1;
R^{X} is H;
W is a 6-membered heterocycle comprising a single N heteroatom, optionally substituted by either alkyl or oxo, or both alkyl and —OH;
and pharmaceutically acceptable salts.

An embodiment of the present invention provides compounds according to formula Ib, wherein the compound of formula Ib is a compound of formula Ic

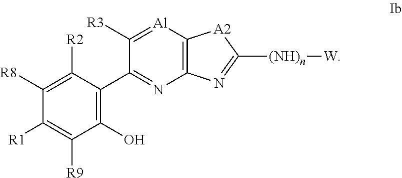

The compounds of formula Ib can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

Also an embodiment of the present invention provides compounds according to formula Ib as described herein and pharmaceutically acceptable salts or esters thereof, in particular compounds according to formula Ib as described herein and pharmaceutically acceptable salts thereof, more particularly compounds according to formula Ib as described herein.

An embodiment of the present invention provides compounds according to formula Ib as described herein, wherein
A¹ is —N— or —CR¹⁰—;
A² is —O—, —NH—, —NCH₃—, or —S—;
R¹ is H, halogen, alkyl, haloalkyl, haloalkoxy, cyano, alkoxy, or cycloalkyl;
R⁹ is H or halogen;
or R¹ and R⁹ form a 4 to 6 membered cycloalkyl or heterocycloalkyl ring comprising a single O heteroatom;
R² is H, halogen, alkyl, cyano, haloalkoxy, haloalkyl, alkoxy, or alkoxyalkyl, and R³ is H, halogen, or alkyl, wherein at least one of R² and R³ is not H;
R⁸ is H or halo;
R¹⁰ is selected from
   i. H,
   ii. alkyl,
   iii. hydroxyalkyl,
   iv. cyano,
   v. halogen
   v. haloalkyl,
   vi. alkoxy,
   vii. haloalkoxy,
   viii. a 5-6 membered heterocycle comprising 1 or 2 N atoms optionally substituted with either halo or methyl, or
   ix. NR′R″, wherein R′ and R″ are independently selected from H or alkyl, or R′ and R″ and the N atom to which they are attached form either a 4-6 membered N-containing heterocycle ring or a 5-membered lactam ring;
n can be 0 or 1;
W is a heterocycloalkyl optionally substituted with 1 to 2 substituents independently selected from: oxo, —OH, alkyl, alkylamino, alkylaminoalkyl, amino, aminoalkyl, cyano, cycloalkyl, or cycloalkyl substituted with amino, halogen, dialkylamino, dialkylaminoalkyl, ester, haloalkyl, hydroxyalkyl, sulfonyl or pyrrolidinylalkyl, or
W is a 3-to-6-membered cycloalkyl optionally substituted with 1 to 2 substituents independently selected from —OH, alkyl, alkylamino, amino, dialkylamino, halogen, haloalkyl and tert-Butyl N-methylcarbamate;
and pharmaceutically acceptable salts.

An embodiment of the present invention provides compounds according to formula Ib as described herein, wherein
A¹ is —N— or —CR¹⁰—;
A² is —O—, or —NH;
R¹ is halogen, haloalkyl, cyano, alkoxy or cycloalkyl;
R⁹ is H or halogen;
or R¹ and R⁹ form a 5-membered heterocycle ring comprising a single O heteroatom;
R² is H, alkyl, cyano, haloalkyl, alkoxy, or alkoxyalkyl, and R³ is H, or alkyl, wherein at least one of R² and R³ is not H;
R⁸ is H;
R¹⁰ is selected from H or methyl;
n can be 0 or 1;
W is selected from ring systems:

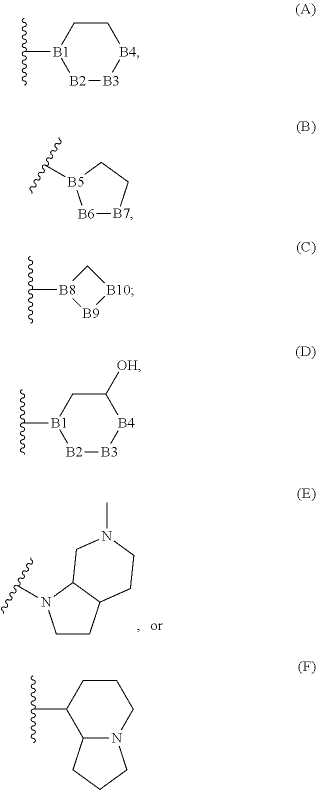

wherein,
   B¹ is —N— or —CH—;
   B² is —CHR⁴— or —NCH₃;
   B³ is —CHR⁵—, —O—, or —NR⁶—
   B⁴ is —O—, —CH₂—, —NH—, —C(═O)—;
   B⁵ is —CH—;
   B⁶ is —NH—;
   B⁷ is —CH₂—;
   B⁸ is —N—;
   B⁹ is —CH₂—;
   B¹⁰ is —CHR⁷—;
   R⁴ is H or dialkylaminoalkyl;
   R⁵ is H, —OH, alkylamino, or hydroxyalkyl;
   R⁶ is H or alkyl;
   R⁷ is —OH;
wherein W can only have a maximum of 2 heteroatoms and 2 non-hydrogen substituents;
wherein if W is ring system (C), ring system (E), or B¹ is —N—, then n is 0;
and pharmaceutical acceptable salts.

An embodiment of the present invention provides compounds according to formula Ib as described herein, wherein
A¹ is —N— or —CR¹⁰—;
A² is —O—, or —NH;
R¹ is halogen, haloalkyl, cyano, or alkoxy;
R⁹ is H or halogen;
or R¹ and R⁹ form a 5-membered heterocycle ring comprising a single O heteroatom;
R² is H, alkyl, cyano, haloalkyl, or alkoxyalkyl, and R³ is H, or alkyl, wherein at least one of R² and R³ is not H;
R⁸ is H;
R¹⁰ is selected from H or methyl;
n can be 0 or 1;
W is selected from ring systems:

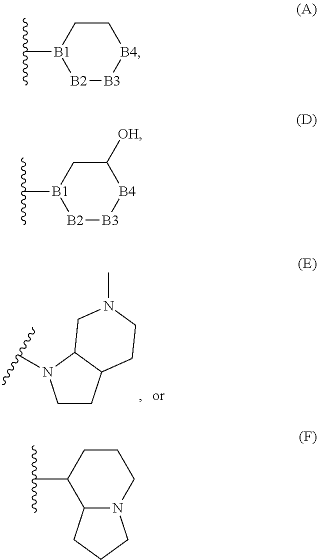

wherein,
   B¹ is —CH—;
   B² is —CHR⁴—;
   B³ is —NR⁶—
   B⁴ is —CH₂—, or —C(═O)—;
   R⁴ is H;
   R⁶ is H or alkyl;
wherein W can only have a maximum of 2 heteroatoms and 2 non-hydrogen substituents;
wherein if W is ring system (E) then n is 0;
and pharmaceutical acceptable salts.

An embodiment of the present invention provides compounds according to formula Ib as described herein, wherein
A¹ is —N— or —CR¹⁰—;
A² is —O—;
R¹ is halogen, haloalkyl, cyano, or alkoxy;
R⁹ is H or halogen;
R² is alkyl, cyano, or alkoxyalkyl, and R³ is H;
R⁸ is H;
R¹⁰ is H;
n can be 0 or 1;
W is selected from ring systems:

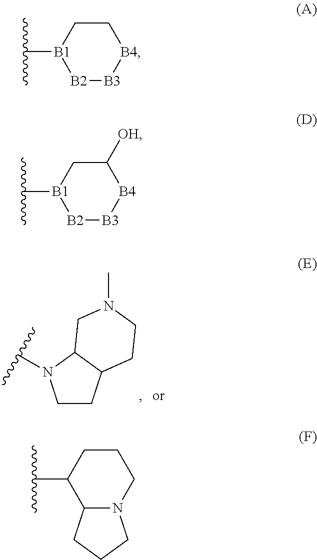

wherein,
   B¹ is —CH—;
   B² is —CHR⁴—;
   B³ is —NR⁶—
   B⁴ is —CH₂—, or —C(═O)—;
   R⁴ is H;
   R⁶ is H or alkyl;
wherein W can only have a maximum of 2 heteroatoms and 2 non-hydrogen substituents;
wherein if W is ring system (E) then n is 0;
and pharmaceutical acceptable salts.

An embodiment of the present invention provides compounds according to formula Ib as described herein, wherein
A¹ is —N— or —CR¹⁰—;
A² is —O—;
R¹ is halogen, haloalkyl, or cyano;
R⁹ is H;
R² is alkyl and R³ is H;
R⁸ is H;
R¹⁰ is H;
n can be 0 or 1;
W is selected from ring systems:

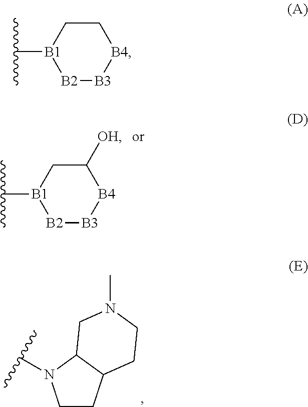

wherein,
   B¹ is —CH—;
   B² is —CHR⁴—;
   B³ is —NR⁶—
   B⁴ is —CH₂—;
   R⁴ is H;
   R⁶ is alkyl;
wherein W can only have a maximum of 2 heteroatoms and 2 non-hydrogen substituents;
wherein if W is ring system (E) then n is 0;
and pharmaceutical acceptable salts.

An embodiment of the present invention provides compounds according to formula Ib as described herein, wherein
A¹ is —CR¹⁰—;
A² is —O—;
R¹ is halogen or cyano;
R⁹ is H;
R² is alkyl and R³ is H;
R⁸ is H;
R¹⁰ is H;
n can be 0 or 1;
W is selected from ring systems:

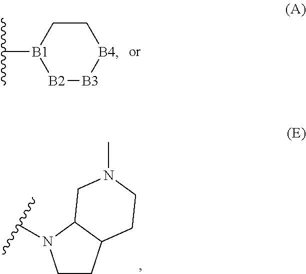

wherein,
   B¹ is —CH—;
   B² is —CHR⁴—;
   B³ is —NR⁶—
   B⁴ is —CH₂—;
   R⁴ is H;
   R⁶ is alkyl;
wherein W can only have a maximum of 2 heteroatoms and 1 non-hydrogen substituents;
wherein if W is ring system (E) then n is 0;
and pharmaceutical acceptable salts.

An embodiment of the present invention provides compounds according to formula I, wherein the compound of formula I is a compound of formula Ib

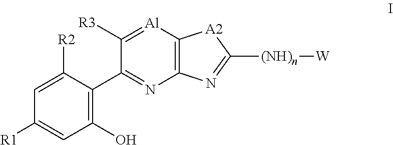

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein
A¹ is —N— or —CH—;
A² is —O—, —NH—, —NCH₃—, or —S—;
R¹ is halogen, alkyl, haloalkyl, or haloalkoxy;
R² is H, halogen, or alkyl and R³ is H, halogen, or alkyl, wherein at least one of R² and R³ is not H;
n can be 0 or 1;
W is a heterocycle ring optionally substituted with 1 to 2 substituents independently selected from: oxo, —OH, alkyl, alkylamino, alkylaminoalkyl, amino, aminoalkyl, cycloalkyl, cycloalkyl substituted with halogen, dialkylamino, dialkylaminoalkyl, haloalkyl, hydroxyalkyl, and pyrrolidinylalkyl, or
W is a 4-to-6-membered cycloalkyl optionally substituted with 1 to 2 substituents independently selected from —OH, alkyl, alkylamino, amino, dialkylamino, halogen, haloalkyl and tert-Butyl N-methylcarbamate;
and pharmaceutically acceptable salts.

An embodiment of the present invention provides compounds according to formula I as described herein, wherein
A¹ is —N— or —CH—;
A² is —O—, —NH—, —NCH₃—, or —S—;
R¹ is halogen, alkyl, haloalkyl, or haloalkoxy;
R² is H, halogen, or alkyl and R³ is H, halogen, or alkyl, wherein at least one of R² and R³ is not H;
n can be 0 or 1;
W is ring system

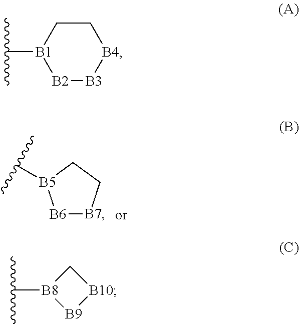

B¹ is —N— or —CH—;
B² is —CHR⁴— or —NCH₃;
B³ is —CHR⁵—, —O—, or —NR⁶—
B⁴ is —O—, —CH₂—, —NH—, —C(═O)—;
B⁵ is —CH—;
B⁶ is —NH—;
B⁷ is —CH₂—;
B⁸ is —N—;
B⁹ is —CH₂—;
B¹⁰ is —CHR⁷—;
R⁴ is H, dialkylaminoalkyl or hydroxyalkyl;
R⁵ is H, —OH, alkylamino, dialkylaminoalkyl or hydroxyalkyl;
R⁶ is H or alkyl;
R⁷ is —OH;
wherein W can only have a maximum of 2 heteroatoms and 2 non-hydrogen substituents;
wherein if W is ring system C or B¹ is —N—, then n is 0;
and pharmaceutical acceptable salts.

An embodiment of the present invention provides compounds according to formula I as described herein, wherein
A¹ is —CH—;
A² is —O—, or —NH—;
R¹ is haloalkyl or halogen;
R² is alkyl and R³ is H;
n can be 0 or 1;
W is ring system

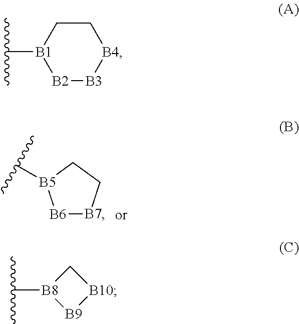

B¹ is —N— or —CH—;
B² is —CHR⁴— or —NCH₃;
B³ is —CHR⁵—, —O—, or —NR⁶—
B⁴ is —O—, —CH₂—, —NH—, —C(═O)—;
B⁵ is —CH—;
B⁶ is —NH—;
B⁷ is —CH₂—;
B⁸ is —N—;
B⁹ is —CH₂—;
B¹⁰ is —CHR⁷—;
R⁴ is H, or dialkylaminoalkyl;
R⁵ is H, —OH, alkylamino, or hydroxyalkyl;
R⁶ is H or alkyl;
R⁷ is —OH;
wherein W can only have a maximum of 2 heteroatoms and 2 non-hydrogen substituents;
wherein if W is ring system C or B¹ is —N—, then n is 0;
and pharmaceutical acceptable salts.

An embodiment of the present invention provides compounds according to formula I as described herein, wherein
A¹ is —CH—;
A² is —O—, or —NH—;
R¹ is haloalkyl or halogen;
R² is alkyl and R³ is H;
n can be 0 or 1;
W is ring system

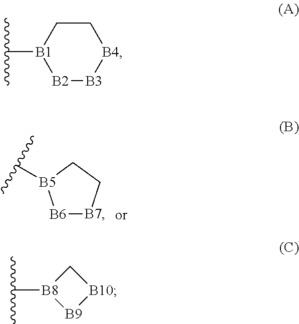

B¹ is —N— or —CH—;
B² is —CHR⁴—;
B³ is —CHR⁵—, or —NR⁶—
B⁴ is —O—, —CH₂—, —NH—, —C(═O)—;
B⁵ is —CH—;
B⁶ is —NH—;
B⁷ is —CH₂—;
B⁸ is —N—;
B⁹ is —CH₂—;
B¹⁰ is —CHR⁷—;
R⁴ is H, or dialkylaminoalkyl;
R⁵ is H, —OH, alkylamino, or hydroxyalkyl;
R⁶ is H or alkyl;
R⁷ is —OH;
wherein W can only have a maximum of 2 heteroatoms and 1 non-hydrogen substituents;
wherein if W is ring system C or B¹ is —N—, then n is 0;
and pharmaceutical acceptable salts.

An embodiment of the present invention provides compounds according to formula I as described herein, wherein
A¹ is —CH—;
A² is —O—, or —NH—;
R¹ is haloalkyl;
R² is alkyl and R³ is H;
n can be 0 or 1;
W is ring system

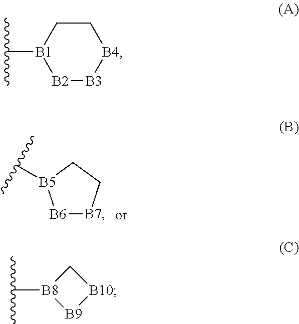

B¹ is —N— or —CH—;
B² is —CHR⁴—;
B³ is —CHR⁵—, or —NR⁶—
B⁴ is —O—, —CH₂—, —NH—, —C(═O)—;
B⁵ is —CH—;
B⁶ is —NH—;
B⁷ is —CH₂—;
B⁸ is —N—;
B⁹ is —CH₂—;
B¹⁰ is —CHR⁷—;
R⁴ is H, or dialkylaminoalkyl;
R⁵ is H, —OH, alkylamino, or hydroxyalkyl;
R⁶ is H or alkyl;
R⁷ is —OH;
wherein W can only have a maximum of 2 heteroatoms and 1 non-hydrogen
substituents;
wherein if W is ring system C or B¹ is —N—, then n is 0;
and pharmaceutical acceptable salts.

An embodiment of the present invention provides compounds according to formula I as described herein, wherein
A¹ is —CH—;
A² is —O—, or —NH—;
R¹ is haloalkyl;
R² is alkyl and R³ is H;
n can be 0 or 1;
W is ring system

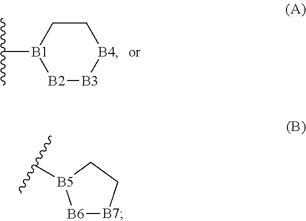

B¹ is —N— or —CH—;
B² is —CHR⁴—;
B³ is —CHR⁵—, or —NR⁶—
B⁴ is —O—, —CH₂—, —NH—, —C(═O)—;
B⁵ is —CH—;
B⁶ is —NH—;
B⁷ is —CH₂—;
R⁴ is H, or dialkylaminoalkyl;
R⁵ is H, —OH, alkylamino, or hydroxyalkyl;
R⁶ is H or alkyl;
wherein W can only have a maximum of 2 heteroatoms and 1 non-hydrogen substituents;
wherein if B¹ is —N—, then n is 0;
and pharmaceutical acceptable salts.

An embodiment of the present invention provides compounds according to formula I as described herein, wherein
A¹ is —CH—;
A² is —O—, or —NH—;
R¹ is haloalkyl;
R² is alkyl and R³ is H;
n can be 0 or 1;
W is ring system

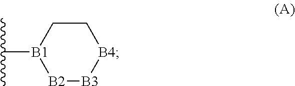

B¹ is —N— or —CH—;
B² is —CHR⁴—;
B³ is —CHR⁵—, or —NR⁶—
B⁴ is —O—, or —CH₂—;
R⁴ is H, or dialkylaminoalkyl;
R⁵ is H;
R⁶ is alkyl;
wherein W can only have a maximum of 2 heteroatoms and 1 non-hydrogen substituents;
wherein if B¹ is —N—, then n is 0;
and pharmaceutical acceptable salts.

An embodiment of the present invention provides compounds according to formula I as described herein, wherein
A¹ is —CH—;
A² is —O—;
R¹ is haloalkyl;
R² is alkyl and R³ is H;
n is 1;
W is ring system

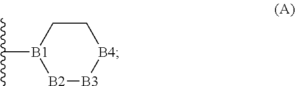

B¹ is-CH—;
B² is —CHR⁴—;
B³ is —NR⁶—
B⁴ is —CH₂—;
R⁴ is H;
R⁶ is alkyl;
and pharmaceutical acceptable salts.

Particular examples of compounds of formula Ic as described herein are selected from
2-[2-[(3aS,7aR)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol or 2-[2-[(3aR,7aS)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol;
2-[2-[(3aR,7aS)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol or 2-[2-[(3aS,7aR)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol;
(rac)-3-Methyl-2-[2-(tetrahydrofuran-3-ylamino)oxazolo[4,5-b]pyridin-5-yl]-5-(trifluoromethyl)phenol;
5-Chloro-3-methyl-2-[2-[[rac-(8S,8aR)-1,2,3,5,6,7,8,8a-octahydroindolizin-8-yl]amino]oxazolo[4,5-b]pyridin-5-yl]phenol;
2-[2-[[(8S,8aR)-1,2,3,5,6,7,8,8a-Octahydroindolizin-8-yl]amino]oxazolo[4,5-b]pyridin-5-yl]-5-chloro-3-methyl-phenol or 2-[2-[[(8R,8aS)-1,2,3,5,6,7,8,8a-Octahydroindolizin-8-yl]amino]oxazolo[4,5-b]pyridin-5-yl]-5-chloro-3-methyl-phenol;
2-[2-[[(8R,8aS)-1,2,3,5,6,7,8,8a-Octahydroindolizin-8-yl]amino]oxazolo[4,5-b]pyridin-5-yl]-5-chloro-3-methyl-phenol or 2-[2-[[(8S,8aR)-1,2,3,5,6,7,8,8a-Octahydroindolizin-8-yl]amino]oxazolo[4,5-b]pyridin-5-yl]-5-chloro-3-methyl-phenol;
5-Chloro-3-methyl-2-[2-(6-methyl-3,4a,5,7,8,8a-hexahydro-2H-pyrido[4,3-b][1,4]oxazin-4-yl)oxazolo[4,5-b]pyridin-5-yl]phenol;
2-[2-(2-Azabicyclo[2.1.1]hexan-1-ylmethylamino)oxazolo[4,5-b]pyridin-5-yl]-3-methyl-5-(trifluoromethyl)phenol;dihydrochloride;
5-Chloro-3-methyl-2-[2-[[(3R)-1-methyl-3-piperidyl]amino]oxazolo[4,5-b]pyridin-5-yl]phenol;
2-[2-[[(3R)-1-Ethyl-3-piperidyl]amino]oxazolo[4,5-b]pyridin-5-yl]-5-fluoro-3-methyl-phenol;
5-Chloro-3-methyl-2-[2-[[(3R)-1-propyl-3-piperidyl]amino]oxazolo[4,5-b]pyridin-5-yl]phenol;
5-Chloro-2-[2-[[(3R)-1-cyclobutyl-3-piperidyl]amino]oxazolo[4,5-b]pyridin-5-yl]-3-methyl-phenol;
5-[2-[[(3R)-1-Ethyl-3-piperidyl]amino]oxazolo[4,5-b]pyridin-5-yl]-6-methyl-2,3-dihydrobenzofuran-4-ol;
(3S,5R)-5-[[5-(4-Chloro-2-hydroxy-6-methyl-phenyl)oxazolo[4,5-b]pyridin-2-yl]amino]-1-methyl-piperidin-3-ol;
5-Fluoro-3-methyl-2-[2-[[(3R)-1-methyl-3-piperidyl]amino]oxazolo[4,5-b]pyridin-5-yl]phenol;
(3S,5R)-1-Ethyl-5-[[5-[2-hydroxy-6-methyl-4-(trifluoromethyl)phenyl]oxazolo[4,5-b]pyridin-2-yl]amino]piperidin-3-ol;
2-[2-[[(3R)-1-(2-Hydroxyethyl)-3-piperidyl]amino]oxazolo[4,5-b]pyridin-5-yl]-3-methyl-5-(trifluoromethyl)phenol;
2-[2-[[(3R)-1-Ethyl-3-piperidyl]amino]oxazolo[4,5-b]pyridin-5-yl]-3-fluoro-5-(trifluoromethyl)phenol;
5-Chloro-2-[2-[[(3R,5S)-1-ethyl-5-fluoro-3-piperidyl]amino]oxazolo[4,5-b]pyridin-5-yl]-3-methyl-phenol;
2-[2-[[(1R,2R)-2-(Dimethylamino)cyclopropyl]amino]oxazolo[4,5-b]pyridin-5-yl]-3-methyl-5-(trifluoromethyl)phenol;
3-Hydroxy-4-[2-[[(3R,5S)-5-hydroxy-1-methyl-3-piperidyl]amino]oxazolo[4,5-b]pyridin-5-yl]-5-methyl-benzonitrile;
cis-2-[2-[(3-Hydroxy-3-methyl-cyclobutyl)methylamino]oxazolo[4,5-b]pyridin-5-yl]-3-methyl-5-(trifluoromethyl)phenol;
trans-2-[2-[(3-Hydroxy-3-methyl-cyclobutyl)methylamino]oxazolo[4,5-b]pyridin-5-yl]-3-methyl-5-(trifluoromethyl)phenol;
5-Chloro-2-[2-[(1-ethylazetidin-3-yl)methylamino]oxazolo[4,5-b]pyridin-5-yl]-3-methyl-phenol;
1-[5-(4-Chloro-2-hydroxy-6-methyl-phenyl)-2-[[rac-(3R)-1-methyl-3-piperidyl]amino]oxazolo[4,5-b]pyridin-7-yl]pyrrolidin-2-one;
2-[2-[[(3R)-1-(3-Hydroxycyclobutyl)-3-piperidyl]amino]oxazolo[4,5-b]pyridin-5-yl]-3-methyl-5-(trifluoromethyl)phenol;
2-[2-(1,3,3a,4,6,6a-Hexahydrofuro[3,4-c]pyrrol-5-yl)oxazolo[4,5-b]pyridin-5-yl]-3-methyl-5-(trifluoromethyl)phenol;
5-Chloro-3-methyl-2-[2-[[(3R)-1-(oxetan-3-yl)-3-piperidyl]amino]oxazolo[4,5-b]pyridin-5-yl]phenol;
3-Methyl-2-[2-[rel-(4aR,7aR)-3,4a,5,6,7,7a-hexahydro-2H-pyrrolo[3,4-b][1,4]oxazin-4-yl]oxazolo[4,5-b]pyridin-5-yl]-5-(trifluoromethyl)phenol;2,2,2-trifluoroacetic acid;
2-[2-[(4aS,7aS)-3,4a,5,6,7,7a-Hexahydro-2H-pyrrolo[3,4-b][1,4]oxazin-4-yl]oxazolo[4,5-b]pyridin-5-yl]-3-methyl-5-(trifluoromethyl)phenol;
2-[2-[(4aR,7aR)-3,4a,5,6,7,7a-Hexahydro-2H-pyrrolo[3,4-b][1,4]oxazin-4-yl]oxazolo[4,5-b]pyridin-5-yl]-3-methyl-5-(trifluoromethyl)phenol;
N-(1,2,3,5,6,7,8,8a-Octahydroindolizin-6-yl)-5-chloro-oxazolo[4,5-b]pyridin-2-amine;
3-Methyl-2-[2-[[(2S)-1-methylazetidin-2-yl]methylamino]oxazolo[4,5-b]pyridin-5-yl]-5-(trifluoromethyl)phenol or 3-methyl-2-[2-[[(2R)-1-methylazetidin-2-yl]methylamino]oxazolo[4,5-b]pyridin-5-yl]-5-(trifluoromethyl)phenol;
3-Methyl-2-[2-[[(2R)-1-methylazetidin-2-yl]methylamino]oxazolo[4,5-b]pyridin-5-yl]-5-(trifluoromethyl)phenol or 3-methyl-2-[2-[[(2S)-1-methylazetidin-2-yl]methylamino]oxazolo[4,5-b]pyridin-5-yl]-5-(trifluoromethyl)phenol;
3-Methyl-2-[2-[(1-methylazetidin-2-yl)methylamino]oxazolo[4,5-b]pyridin-5-yl]-5-(trifluoromethyl)phenol;
5-Chloro-2-[2-(1H-imidazol-5-ylmethylamino)oxazolo[4,5-b]pyridin-5-yl]-3-methyl-phenol;
(3RS,5R)-5-[[5-(4-Chloro-2-hydroxy-6-methyl-phenyl)oxazolo[4,5-b]pyridin-2-yl]amino]-1-ethyl-piperidin-3-ol;formic acid;
3,6-Dimethyl-2-[2-[[(3R)-1-methyl-3-piperidyl]amino]oxazolo[4,5-b]pyridin-5-yl]phenol;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
4-[2-[[(3R,5R)-1-Ethyl-5-hydroxy-3-piperidyl]amino]oxazolo[4,5-b]pyridin-5-yl]-3-hydroxy-5-methyl-benzonitrile;
4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile or 4-[2-[(3aR,7aS)-6-ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[(3aR,7aS)-6-ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile or 4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
5-[5-(4-Chloro-2-hydroxy-6-methyl-phenyl)oxazolo[4,5-b]pyridin-2-yl]piperidin-2-one;
(5S)-5-[5-(4-Chloro-2-hydroxy-6-methyl-phenyl)oxazolo[4,5-b]pyridin-2-yl]piperidin-2-one or (5R)-5-[5-(4-Chloro-2-hydroxy-6-methyl-phenyl)oxazolo[4,5-b]pyridin-2-yl]piperidin-2-one;
(5R)-5-[5-(4-Chloro-2-hydroxy-6-methyl-phenyl)oxazolo[4,5-b]pyridin-2-yl]piperidin-2-one or (5S)-5-[5-(4-chloro-2-hydroxy-6-methyl-phenyl)oxazolo[4,5-b]pyridin-2-yl]piperidin-2-one;
5-Chloro-2-[2-[[(3R)-1-(3,3-difluoropropyl)-3-piperidyl]amino]oxazolo[4,5-b]pyridin-5-yl]-3-methyl-phenol;
5-Chloro-3-methyl-2-[2-[[(3R)-1-[(3-hydroxycyclobutyl)methyl]-3-piperidyl]amino]oxazolo[4,5-b]pyridin-5-yl]phenol;
5-Chloro-2-[2-(1H-imidazol-2-ylmethylamino)oxazolo[4,5-b]pyridin-5-yl]-3-methyl-phenol;
2-[2-[3-(Hydroxymethyl)-1-piperidyl]oxazolo[4,5-b]pyridin-5-yl]-3-methyl-5-(trifluoromethyl)phenol;
2-[2-[4-(Hydroxymethyl)-1-piperidyl]oxazolo[4,5-b]pyridin-5-yl]-3-methyl-5-(trifluoromethyl)phenol;
2-[2-(1-Ethyl-1,7-diazaspiro[3.5]nonan-7-yl)oxazolo[4,5-b]pyridin-5-yl]-3-methyl-5-(trifluoromethyl)phenol;
3-Hydroxy-5-methyl-4-[2-[rac trans-6-ethyl-2,3,4a,5,7,7a-hexahydropyrrolo[3,4-b][1,4]-oxazin-4-yl]oxazolo[4,5-b]pyridin-5-yl]benzonitrile;
3-Hydroxy-5-methyl-4-[2-(7-methyl-2,3,4,4a,5,6,8,8a-octahydro-1,7-naphthyridin-1-yl)oxazolo[4,5-b]pyridin-5-yl]benzonitrile;
2-Hydroxy-4-methyl-3-[2-[[(3R)-1-methyl-3-piperidyl]amino]oxazolo[4,5-b]pyridin-5-yl]benzonitrile;2,2,2-trifluoroacetic acid;
5-Chloro-3-methyl-2-[2-[(1-methyl-3-piperidyl)methyl]oxazolo[4,5-b]pyridin-5-yl]phenol;
Methyl 4-[(3R)-3-[[5-(4-chloro-2-hydroxy-6-methyl-phenyl)oxazolo[4,5-b]pyridin-2-yl]amino]-1-piperidyl]butanoate;
3-Hydroxy-4-[2-[[(3R)-1-(2-hydroxyethyl)-3-piperidyl]amino]oxazolo[4,5-b]pyridin-5-yl]-5-methyl-benzonitrile;
Methyl 3-[(3R)-3-[[5-(4-chloro-2-hydroxy-6-methyl-phenyl)oxazolo[4,5-b]pyridin-2-yl]amino]-1-piperidyl]propanoate;
5-Chloro-2-[2-[[6-(hydroxymethyl)-1-methyl-3-piperidyl]amino]oxazolo[4,5-b]pyridin-5-yl]-3-methyl-phenol;2,2,2-trifluoroacetic acid;
5-Chloro-2-[2-[(4-isopropylmorpholin-2-yl)methyl]oxazolo[4,5-b]pyridin-5-yl]-3-methyl-phenol;formic acid;
3-Methyl-2-[2-[[(3R)-1-methyl-3-piperidyl]amino]oxazolo[4,5-b]pyridin-5-yl]-5-methylsulfonyl-phenol;2,2,2-trifluoroacetic acid;
5-Chloro-2-[2-[2-methoxyethyl-[(3R)-1-methyl-3-piperidyl]amino]oxazolo[4,5-b]pyridin-5-yl]-3-methyl-phenol;formic acid;
2-[2-[[(3R)-1-Ethylazepan-3-yl]amino]oxazolo[4,5-b]pyridin-5-yl]-3-methyl-5-(trifluoromethyl)phenol;
5-Chloro-2-[2-[(3-hydroxycyclobutyl)methylamino]oxazolo[4,5-b]pyridin-5-yl]-3-methyl-phenol;
5-Chloro-2-[2-[[(3R)-1-(2-hydroxyethyl)-3-piperidyl]amino]oxazolo[4,5-b]pyridin-5-yl]-3-methyl-phenol;
5-Chloro-3-(hydroxymethyl)-2-[2-[[(3R)-1-methyl-3-piperidyl]amino]oxazolo[4,5-b]pyridin-5-yl]phenol;
5-Chloro-2-[2-[[(3R)-1-(3-hydroxypropyl)-3-piperidyl]amino]oxazolo[4,5-b]pyridin-5-yl]-3-methyl-phenol;
5-Chloro-3-methyl-2-[2-[[rac-(3R)-1-(2-hydroxypropyl)-3-piperidyl]amino]oxazolo[4,5-b]pyridin-5-yl]phenol;
5-Chloro-2-[2-[2-hydroxyethyl-[(3R)-1-methyl-3-piperidyl]amino]oxazolo[4,5-b]pyridin-5-yl]-3-methyl-phenol;
5-Chloro-2-[2-[[(3S,4R)-1-ethyl-4-fluoro-3-piperidyl]amino]oxazolo[4,5-b]pyridin-5-yl]-3-methyl-phenol;
5-Chloro-2-[2-[[(3R,4S)-1-ethyl-4-fluoro-3-piperidyl]amino]oxazolo[4,5-b]pyridin-5-yl]-3-methyl-phenol;
(3S,5R)-5-[[5-[4-Chloro-2-hydroxy-6-(methoxymethyl)phenyl]oxazolo[4,5-b]pyridin-2-yl]amino]-1-methyl-piperidin-3-ol;
1-[2-[[(3R)-1-Ethyl-3-piperidyl]amino]-5-[2-hydroxy-6-methyl-4-(trifluoromethyl)phenyl]oxazolo[4,5-b]pyridin-7-yl]azetidin-3-ol;
1-[5-[2-Hydroxy-6-methyl-4-(trifluoromethyl)phenyl]-2-[[(3R)-1-methyl-3-piperidyl]amino]oxazolo[4,5-b]pyridin-7-yl]ethanone;2,2,2-trifluoroacetic acid;
2-[2-[[(3R)-1-Ethyl-3-piperidyl]amino]-7-(methylamino)oxazolo[4,5-b]pyridin-5-yl]-3-methyl-5-(trifluoromethyl)phenol;
2-[2-[[(3R)-1-Ethyl-3-piperidyl]amino]-7-methoxy-oxazolo[4,5-b]pyridin-5-yl]-3-methyl-5-(trifluoromethyl)phenol;
2-[7-(1-Hydroxy-1-methyl-ethyl)-2-[[(3R)-1-methyl-3-piperidyl]amino]oxazolo[4,5-b]pyridin-5-yl]-3-methyl-5-(trifluoromethyl)phenol;2,2,2-trifluoroacetic acid;
2-[7-(1-Hydroxyethyl)-2-[[(3R)-1-methyl-3-piperidyl]amino]oxazolo[4,5-b]pyridin-5-yl]-3-methyl-5-(trifluoromethyl)phenol;
3-Chloro-4-[2-[[(3R)-1-ethyl-3-piperidyl]amino]oxazolo[4,5-b]pyridin-5-yl]-5-hydroxy-benzonitrile;2,2,2-trifluoroacetic acid;
4-[2-[[(1R,2R)-2-(Dimethylamino)cyclopentyl]amino]oxazolo[4,5-b]pyridin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;
5-Chloro-2-[2-[[(1R,2R)-2-(dimethylamino)cyclopentyl]amino]oxazolo[4,5-b]pyridin-5-yl]-3-methyl-phenol;
5-Chloro-2-[2-[[(3R)-1-ethyl-3-piperidyl]amino]oxazolo[4,5-b]pyrazin-5-yl]-3-methyl-phenol;2,2,2-trifluoroacetic acid;
5-Chloro-2-[2-[[(3R)-1-ethyl-3-piperidyl]amino]oxazolo[4,5-b]pyridin-5-yl]-3-(trifluoromethoxy)phenol;2,2,2-trifluoroacetic acid;
3-Hydroxy-5-methyl-4-[2-[[(2S)-2-hydroxy-1,2,3,5,6,7,8,8a-octahydroindolizin-8-yl]amino]oxazolo[4,5-b]pyridin-5-yl]benzonitrile;2,2,2-trifluoroacetic acid;
2-[7-(Dimethylamino)-2-[[(3R)-1-ethyl-3-piperidyl]amino]oxazolo[4,5-b]pyridin-5-yl]-3-methyl-5-(trifluoromethyl)phenol;
2-[[(3R)-1-Ethyl-3-piperidyl]amino]-5-[2-hydroxy-6-methyl-4-(trifluoromethyl)phenyl]oxazolo[4,5-b]pyridine-7-carbonitrile;
5-chloro-2-[2-[[(3R)-1-[[rac trans-2-hydroxycyclobutyl]methyl]-3-piperidyl]amino]oxazolo[4,5-b]pyridin-5-yl]-3-methyl-phenol or 5-chloro-2-[2-[[(3R)-1-[[rac cis-2-hydroxycyclobutyl]methyl]-3-piperidyl]amino]oxazolo[4,5-b]pyridin-5-yl]-3-methyl-phenol;
5-chloro-2-[2-[[(3R)-1-[[rac trans-2-hydroxycyclobutyl]methyl]-3-piperidyl]amino]oxazolo[4,5-b]pyridin-5-yl]-3-methyl-phenol or 5-chloro-2-[2-[[(3R)-1-[[rac cis-2-hydroxycyclobutyl]methyl]-3-piperidyl]amino]oxazolo[4,5-b]pyridin-5-yl]-3-methyl-phenol;
2-[2-[[(3R)-1-Ethyl-3-piperidyl]amino]oxazolo[4,5-b]pyridin-5-yl]-3-hydroxy-benzonitrile;
3-Methyl-2-[2-[(2-methyl-2-azabicyclo[2.2.2]octan-4-yl)amino]oxazolo[4,5-b]pyridin-5-yl]-5-(trifluoromethyl)phenol;
5-(Difluoromethyl)-3-methyl-2-[2-[[(3R)-1-methyl-3-piperidyl]amino]oxazolo[4,5-b]pyridin-5-yl]phenol;
5-(Difluoromethyl)-2-[2-[[(3R)-1-ethyl-3-piperidyl]amino]oxazolo[4,5-b]pyridin-5-yl]-3-methyl-phenol;2,2,2-trifluoroacetic acid;
5-(Difluoromethyl)-2-[2-[[(3R)-1-ethyl-3-piperidyl]amino]oxazolo[4,5-b]pyridin-5-yl]-3-methyl-phenol;
2-[2-[[(3R)-1-Ethyl-3-piperidyl]amino]oxazolo[4,5-b]pyridin-5-yl]-3,5-dimethyl-phenol;
3-[[5-(4-Chloro-2-hydroxy-6-methyl-phenyl)oxazolo[4,5-b]pyridin-2-yl]amino]-1-ethyl-piperidin-4-ol;
2-[2-[[(3R)-1-Ethyl-3-piperidyl]amino]oxazolo[4,5-b]pyridin-5-yl]-3-methyl-phenol;
5-Fluoro-2-[2-[[(3R)-1-methyl-3-piperidyl]amino]oxazolo[4,5-b]pyridin-5-yl]-3-(trifluoromethyl)phenol;2,2,2-trifluoroacetic acid;
5-Chloro-2-[6-chloro-2-[[(3R)-1-ethyl-3-piperidyl]amino]oxazolo[4,5-b]pyridin-5-yl]phenol;
3,5-Dimethyl-2-[2-[[(3R)-1-methyl-3-piperidyl]amino]oxazolo[4,5-b]pyridin-5-yl]phenol;2,2,2-trifluoroacetic acid;
3-Chloro-2-[2-[[(3R)-1-ethyl-3-piperidyl]amino]oxazolo[4,5-b]pyridin-5-yl]-5-fluoro-phenol;2,2,2-trifluoroacetic acid;
5-(Difluoromethoxy)-2-[2-[[(3R)-1-ethyl-3-piperidyl]amino]oxazolo[4,5-b]pyridin-5-yl]-3-methyl-phenol;2,2,2-trifluoroacetic acid;
2-[2-[(1-Ethylpyrrolidin-3-yl)amino]oxazolo[4,5-b]pyridin-5-yl]-3-methyl-5-(trifluoromethyl)phenol;
3,5-Dichloro-2-[2-[[(3R)-1-ethyl-3-piperidyl]amino]oxazolo[4,5-b]pyridin-5-yl]phenol;2,2,2-trifluoroacetic acid;
2-[2-[[(3R)-1-Ethyl-3-piperidyl]amino]-6-fluoro-TH-imidazo[4,5-b]pyridin-5-yl]-5-(trifluoromethyl)phenol;
(3S,5R)-5-[[5-(4-Chloro-2-hydroxy-6-methyl-phenyl)oxazolo[4,5-b]pyridin-2-yl]amino]-1-ethyl-piperidin-3-ol;
3-Methyl-2-[2-[4-(methylamino)tetrahydropyran-4-yl]oxazolo[4,5-b]pyridin-5-yl]-5-(trifluoromethyl)phenol;2,2,2-trifluoroacetic acid;
5-Chloro-2-[2-[[(3R)-1-ethyl-3-piperidyl]amino]-3H-imidazo[4,5-b]pyridin-5-yl]-3-methyl-phenol;
2-[2-[[(3R)-1-Ethyl-3-piperidyl]amino]oxazolo[4,5-b]pyridin-5-yl]-3-methyl-5-(trifluoromethoxy)phenol;2,2,2-trifluoroacetic acid;
2-[2-(1-Ethyl-3-piperidyl)oxazolo[4,5-b]pyridin-5-yl]-3-methyl-5-(trifluoromethyl)phenol;
4-[2-[[(8R,8aR)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8S,8aS)-1,2,3,5,6,7,8,8a-octahydroindolizin-8-yl]amino]oxazolo[4,5-b]pyridin-5-yl]-3-hydroxy-5-methyl-benzonitrile;
4-[2-[[(8S,8aS)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8R,8aR)-1,2,3,5,6,7,8,8a-octahydroindolizin-8-yl]amino]oxazolo[4,5-b]pyridin-5-yl]-3-hydroxy-5-methyl-benzonitrile;

and pharmaceutically acceptable salts thereof.

Also particular examples of compounds of formula Ic as described herein are selected from
(rac)-2-[2-[(1-Ethyl-3-piperidyl)amino]oxazolo[4,5-b]pyridin-5-yl]-3-methyl-5-(trifluoromethyl)phenol;
(rac)-2-[2-[3-[(Dimethylamino)methyl]morpholin-4-yl]oxazolo[4,5-b]pyridin-5-yl]-3-methyl-5-(trifluoromethyl)phenol;
2-[2-[(3R or 3S)-3-[(Dimethylamino)methyl]morpholin-4-yl]oxazolo[4,5-b]pyridin-5-yl]-3-methyl-5-(trifluoromethyl)phenol;
2-[2-[(3S or 3R)-3-[(Dimethylamino)methyl]morpholin-4-yl]oxazolo[4,5-b]pyridin-5-yl]-3-methyl-5-(trifluoromethyl)phenol;
(rac)-1-[5-[2-Hydroxy-6-methyl-4-(trifluoromethyl)phenyl]oxazolo[4,5-b]pyridin-2-yl]piperidin-3-ol;
(3R or 3S)-1-[5-[2-Hydroxy-6-methyl-4-(trifluoromethyl)phenyl]oxazolo[4,5-b]pyridin-2-yl]piperidin-3-ol;
(3S or 3R)-1-[5-[2-Hydroxy-6-methyl-4-(trifluoromethyl)phenyl]oxazolo[4,5-b]pyridin-2-yl]piperidin-3-ol;
3-Methyl-2-(2-morpholinooxazolo[4,5-b]pyridin-5-yl)-5-(trifluoromethyl)phenol;
(rac)-[2-[2-(Hydroxymethyl)morpholin-4-yl]oxazolo[4,5-b]pyridin-5-yl]-3-methyl-5-(trifluoromethyl)phenol;
3-Methyl-2-(2-piperazin-1-yloxazolo[4,5-b]pyridin-5-yl)-5-(trifluoromethyl)phenol;
3-Methyl-2-[2-[[3-(methylamino)cyclohexyl]amino]oxazolo[4,5-b]pyridin-5-yl]-5-(trifluoromethyl)phenol;
3-Methyl-2-(2-piperazin-1-yl-1H-imidazo[4,5-b]pyridin-5-yl)-5-(trifluoromethyl)phenol;
(rac)-5-(5-(2-Hydroxy-6-methyl-4-(trifluoromethyl)phenyl)oxazolo[4,5-b]pyridin-2-yl) piperidin-2-one;
(rac)-3-Methyl-2-[2-[3-(methylamino)-1-piperidyl]oxazolo[4,5-b]pyridin-5-yl]-5-(trifluoromethyl) phenol;
(rac)-3-Methyl-2-[2-(tetrahydropyran-3-ylamino)oxazolo[4,5-b]pyridin-5-yl]-5-(trifluoromethyl)phenol;
1-[5-[2-Hydroxy-6-methyl-4-(trifluoromethyl)phenyl]oxazolo[4,5-b]pyridin-2-yl]azetidin-3-ol;
2-[2-[(3-Hydroxycyclohexyl)amino]oxazolo[4,5-b]pyridin-5-yl]-3-methyl-5-(trifluoromethyl)phenol;
2-[2-[[(3R)-1-Ethyl-3-piperidyl]amino]oxazolo[4,5-b]pyridin-5-yl]-3-methyl-5-(trifluoromethyl)phenol;
3-Methyl-2-[2-[[(3R)-1-methyl-3-piperidyl]amino]oxazolo[4,5-b]pyridin-5-yl]-5-(trifluoromethyl)phenol;
(rac)-3-Methyl-2-(2-(1-methylpiperidin-2-yl)-3H-imidazo[4,5-b]pyridin-5-yl)-5-(trifluoromethyl)phenol;
(rac)-3-Methyl-2-(2-(pyrrolidin-2-yl)-1H-imidazo[4,5-b]pyridin-5-yl)-5-(trifluoro-methyl)phenol;
(rac)-2-[2-[(1-Ethyl-3-piperidyl)amino]-1H-imidazo[4,5-b]pyridin-5-yl]-3-methyl-5-(trifluoromethyl)phenol;
2-[2-[[(3R or 3S)-1-Ethyl-3-piperidyl]amino]-1H-imidazo[4,5-b]pyridin-5-yl]-3-methyl-5-(trifluoromethyl)phenol;
2-[2-[[(3S or 3R)-1-Ethyl-3-piperidyl]amino]-1H-imidazo[4,5-b]pyridin-5-yl]-3-methyl-5-(trifluoromethyl)phenol;

and pharmaceutically acceptable salts thereof.

Also particular examples of compounds of formula Ic as described herein are selected from
5-Chloro-2-[2-[[(3R)-1-ethyl-3-piperidyl]amino]oxazolo[4,5-b]pyridin-5-yl]-3-methyl-phenol;
5-Chloro-3-methyl-2-[2-(6-methyl-3,3a,4,5,7,7a-hexahydro-2H-pyrrolo[2,3-c]pyridin-1-yl)oxazolo[4,5-b]pyridin-5-yl]phenol;
2-[2-[(3aS,7aR or 3aR,7aS)-6-Methyl-3,3a,4,5,7,7a-hexahydro-2H-pyrrolo[2,3-c]pyridin-1-yl]oxazolo[4,5-b]pyridin-5-yl]-5-chloro-3-methyl-phenol;
2-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-5-chloro-3-methyl-phenol;
5-Chloro-2-[2-[[(3R)-1-ethyl-3-piperidyl]amino]oxazolo[4,5-b]pyridin-5-yl]-6-fluoro-3-methyl-phenol;
5-Chloro-2-[2-[[(3R)-1-ethyl-3-piperidyl]amino]oxazolo[4,5-b]pyridin-5-yl]-6-fluoro-3-methyl-phenol;
5-Chloro-2-[2-[[(3R)-1-ethyl-3-piperidyl]amino]oxazolo[4,5-b]pyridin-5-yl]-4-fluoro-3-methyl-phenol;
5-Chloro-2-[2-[[(3R)-1-ethyl-3-piperidyl]amino]oxazolo[4,5-b]pyridin-5-yl]-4-fluoro-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;
5-Chloro-2-[2-[[(3R)-1-ethyl-3-piperidyl]amino]oxazolo[4,5-b]pyridin-5-yl]-3-(methoxymethyl)phenol;
4-[2-[[(3R)-1-Ethyl-3-piperidyl]amino]oxazolo[4,5-b]pyridin-5-yl]-3-hydroxy-5-methyl-benzonitrile;
3-hydroxy-5-Methyl-4-[2-[[(3R)-1-methyl-3-piperidyl]amino]oxazolo[4,5-b]pyridin-5-yl]benzonitrile;
4-[2-[(3aS,7aR or 3aR,7aS)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
4-[2-(1,2,3,5,6,7,8,8a-Octahydroindolizin-8-ylamino)oxazolo[4,5-b]pyridin-5-yl]-3-hydroxy-5-methyl-benzonitrile;
3-(Difluoromethyl)-2-[2-[[(3R)-1-methyl-3-piperidyl]amino]oxazolo[4,5-b]pyridin-5-yl]-5-(trifluoromethyl)phenol;
2-[2-[[(3R)-1-Ethyl-3-piperidyl]amino]oxazolo[4,5-b]pyridin-5-yl]-3-methoxy-5-(trifluoromethyl)phenol;
2-[2-[[(3R)-1-Ethyl-3-piperidyl]amino]oxazolo[4,5-b]pyridin-5-yl]-3-methoxy-5-(trifluoromethyl)phenol;
2-[2-[[(3R)-1-Ethyl-3-piperidyl]amino]oxazolo[4,5-b]pyridin-5-yl]-3-methoxy-5-(trifluoromethyl)phenol;
2-[2-[[(3R)-1-Ethyl-3-piperidyl]amino]oxazolo[4,5-b]pyridin-5-yl]-3-methoxy-5-(trifluoromethyl)phenol;
2-[2-[(1-Ethyl-3-piperidyl)amino]oxazolo[4,5-b]pyrazin-5-yl]-3-methyl-5-(trifluoromethyl)phenol;
2-[2-[(1-Ethyl-3-piperidyl)amino]oxazolo[4,5-b]pyrazin-5-yl]-3-methyl-5-(trifluoromethyl)phenol;
3-Methyl-2-[7-methyl-2-[[(3R)-1-methyl-3-piperidyl]amino]oxazolo[4,5-b]pyridin-5-yl]-5-(trifluoromethyl)phenol;
3-Methyl-2-[7-methyl-2-[[(3R)-1-methyl-3-piperidyl]amino]oxazolo[4,5-b]pyridin-5-yl]-5-(trifluoromethyl)phenol;
5-Methoxy-3-methyl-2-[2-[[(3R)-1-methyl-3-piperidyl]amino]oxazolo[4,5-b]pyridin-5-yl]phenol;
5-[2-[[(3R)-1-Ethyl-3-piperidyl]amino]-6-methyl-oxazolo[4,5-b]pyridin-5-yl]-2,3-dihydrobenzofuran-4-ol;
5-Cyclopropyl-3-methyl-2-[2-[[(3R)-1-methyl-3-piperidyl]amino]oxazolo[4,5-b]pyridin-5-yl]phenol;

and pharmaceutically acceptable salts thereof.

Preferred examples of compounds of formula Ic as described herein are selected from
2-[2-[[(8R,8aS)-1,2,3,5,6,7,8,8a-Octahydroindolizin-8-yl]amino]oxazolo[4,5-b]pyridin-5-yl]-5-chloro-3-methyl-phenol or 2-[2-[[(8S,8aR)-1,2,3,5,6,7,8,8a-Octahydroindolizin-8-yl]amino]oxazolo[4,5-b]pyridin-5-yl]-5-chloro-3-methyl-phenol;
5-[2-[[(3R)-1-Ethyl-3-piperidyl]amino]oxazolo[4,5-b]pyridin-5-yl]-6-methyl-2,3-dihydrobenzofuran-4-ol;
(3S,5R)-5-[[5-(4-Chloro-2-hydroxy-6-methyl-phenyl)oxazolo[4,5-b]pyridin-2-yl]amino]-1-methyl-piperidin-3-ol;
3-Hydroxy-4-[2-[[(3R,5S)-5-hydroxy-1-methyl-3-piperidyl]amino]oxazolo[4,5-b]pyridin-5-yl]-5-methyl-benzonitrile;
3-Methyl-2-[2-[rel-(4aR,7aR)-3,4a,5,6,7,7a-hexahydro-2H-pyrrolo[3,4-b][1,4]oxazin-4-yl]oxazolo[4,5-b]pyridin-5-yl]-5-(trifluoromethyl)phenol;2,2,2-trifluoroacetic acid;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
4-[2-[[(3R,5R)-1-Ethyl-5-hydroxy-3-piperidyl]amino]oxazolo[4,5-b]pyridin-5-yl]-3-hydroxy-5-methyl-benzonitrile;
4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile or 4-[2-[(3aR,7aS)-6-ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
(5R)-5-[5-(4-Chloro-2-hydroxy-6-methyl-phenyl)oxazolo[4,5-b]pyridin-2-yl]piperidin-2-one or (5S)-5-[5-(4-chloro-2-hydroxy-6-methyl-phenyl)oxazolo[4,5-b]pyridin-2-yl]piperidin-2-one;
5-Chloro-3-methyl-2-[2-[(1-methyl-3-piperidyl)methyl]oxazolo[4,5-b]pyridin-5-yl]phenol;
3-Hydroxy-4-[2-[[(3R)-1-(2-hydroxyethyl)-3-piperidyl]amino]oxazolo[4,5-b]pyridin-5-yl]-5-methyl-benzonitrile;
5-Chloro-2-[2-[[(3S,4R)-1-ethyl-4-fluoro-3-piperidyl]amino]oxazolo[4,5-b]pyridin-5-yl]-3-methyl-phenol;
(3S,5R)-5-[[5-[4-Chloro-2-hydroxy-6-(methoxymethyl)phenyl]oxazolo[4,5-b]pyridin-2-yl]amino]-1-methyl-piperidin-3-ol;
2-[2-[[(3R)-1-Ethyl-3-piperidyl]amino]-7-methoxy-oxazolo[4,5-b]pyridin-5-yl]-3-methyl-5-(trifluoromethyl)phenol;
4-[2-[[(1R,2R)-2-(Dimethylamino)cyclopentyl]amino]oxazolo[4,5-b]pyridin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;
2-[[(3R)-1-Ethyl-3-piperidyl]amino]-5-[2-hydroxy-6-methyl-4-(trifluoromethyl)phenyl]oxazolo[4,5-b]pyridine-7-carbonitrile;
5-(Difluoromethyl)-3-methyl-2-[2-[[(3R)-1-methyl-3-piperidyl]amino]oxazolo[4,5-b]pyridin-5-yl]phenol;
5-(Difluoromethyl)-2-[2-[[(3R)-1-ethyl-3-piperidyl]amino]oxazolo[4,5-b]pyridin-5-yl]-3-methyl-phenol;
2-[2-[[(3R)-1-Ethyl-3-piperidyl]amino]oxazolo[4,5-b]pyridin-5-yl]-3,5-dimethyl-phenol;
4-[2-[[(8S,8aS)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8R,8aR)-1,2,3,5,6,7,8,8a-octahydroindolizin-8-yl]amino]oxazolo[4,5-b]pyridin-5-yl]-3-hydroxy-5-methyl-benzonitrile;

and pharmaceutically acceptable salts thereof.

More preferred examples of compounds of formula Ic as described herein are selected from
5-Chloro-2-[2-[[(3R)-1-ethyl-3-piperidyl]amino]oxazolo[4,5-b]pyridin-5-yl]-3-methyl-phenol;
5-Chloro-3-methyl-2-[2-(6-methyl-3,3a,4,5,7,7a-hexahydro-2H-pyrrolo[2,3-c]pyridin-1-yl)oxazolo[4,5-b]pyridin-5-yl]phenol;
2-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-5-chloro-3-methyl-phenol;
4-[2-[[(3R)-1-Ethyl-3-piperidyl]amino]oxazolo[4,5-b]pyridin-5-yl]-3-hydroxy-5-methyl-benzonitrile;
3-hydroxy-5-Methyl-4-[2-[[(3R)-1-methyl-3-piperidyl]amino]oxazolo[4,5-b]pyridin-5-yl]benzonitrile;
4-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
2-[2-[(1-Ethyl-3-piperidyl)amino]oxazolo[4,5-b]pyrazin-5-yl]-3-methyl-5-(trifluoromethyl)phenol;
2-[2-[(1-Ethyl-3-piperidyl)amino]oxazolo[4,5-b]pyrazin-5-yl]-3-methyl-5-(trifluoromethyl)phenol;

and pharmaceutically acceptable salts thereof.

Most preferred examples of compounds of formula Ic as described herein are selected from
4-[2-[[(3R)-1-Ethyl-3-piperidyl]amino]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid salt;
(5R)-5-[5-(4-Chloro-2-hydroxy-6-methyl-phenyl)oxazolo[4,5-b]pyridin-2-yl]piperidin-2-one or (5S)-5-[5-(4-chloro-2-hydroxy-6-methyl-phenyl)oxazolo[4,5-b]pyridin-2-yl]piperidin-2-one;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid;
4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid;

and pharmaceutically acceptable salts thereof.

Also most preferred examples of compounds of formula Ic as described herein are selected from
4-[2-[[(3R,5R)-1-Ethyl-5-hydroxy-3-piperidyl]amino]oxazolo[4,5-b]pyridin-5-yl]-3-hydroxy-5-methyl-benzonitrile;
(5R)-5-[5-(4-Chloro-2-hydroxy-6-methyl-phenyl)oxazolo[4,5-b]pyridin-2-yl]piperidin-2-one or (5S)-5-[5-(4-chloro-2-hydroxy-6-methyl-phenyl)oxazolo[4,5-b]pyridin-2-yl]piperidin-2-one;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid;

and pharmaceutically acceptable salts thereof.

Preferred examples of compounds of formula Ib as described herein are selected from (rac)-5-(5-(2-Hydroxy-6-methyl-4-(trifluoromethyl)phenyl)oxazolo[4,5-b]pyridin-2-yl) piperidin-2-one;
5-Chloro-2-[2-[[(3R)-1-ethyl-3-piperidyl]amino]oxazolo[4,5-b]pyridin-5-yl]-3-methyl-phenol;
5-Chloro-3-methyl-2-[2-(6-methyl-3,3a,4,5,7,7a-hexahydro-2H-pyrrolo[2,3-c]pyridin-1-yl)oxazolo[4,5-b]pyridin-5-yl]phenol;
2-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-5-chloro-3-methyl-phenol;
5-Chloro-2-[2-[[(3R)-1-ethyl-3-piperidyl]amino]oxazolo[4,5-b]pyridin-5-yl]-6-fluoro-3-methyl-phenol;
5-Chloro-2-[2-[[(3R)-1-ethyl-3-piperidyl]amino]oxazolo[4,5-b]pyridin-5-yl]-6-fluoro-3-methyl-phenol;
5-Chloro-2-[2-[[(3R)-1-ethyl-3-piperidyl]amino]oxazolo[4,5-b]pyridin-5-yl]-4-fluoro-3-methyl-phenol;
5-Chloro-2-[2-[[(3R)-1-ethyl-3-piperidyl]amino]oxazolo[4,5-b]pyridin-5-yl]-4-fluoro-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;
5-Chloro-2-[2-[[(3R)-1-ethyl-3-piperidyl]amino]oxazolo[4,5-b]pyridin-5-yl]-3-(methoxymethyl)phenol;
4-[2-[[(3R)-1-Ethyl-3-piperidyl]amino]oxazolo[4,5-b]pyridin-5-yl]-3-hydroxy-5-methyl-benzonitrile;
3-hydroxy-5-Methyl-4-[2-[[(3R)-1-methyl-3-piperidyl]amino]oxazolo[4,5-b]pyridin-5-yl]benzonitrile;
4-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
4-[2-(1,2,3,5,6,7,8,8a-Octahydroindolizin-8-ylamino)oxazolo[4,5-b]pyridin-5-yl]-3-hydroxy-5-methyl-benzonitrile;
2-[2-[(1-Ethyl-3-piperidyl)amino]oxazolo[4,5-b]pyrazin-5-yl]-3-methyl-5-(trifluoromethyl)phenol;
2-[2-[(1-Ethyl-3-piperidyl)amino]oxazolo[4,5-b]pyrazin-5-yl]-3-methyl-5-(trifluoromethyl)phenol;
5-Methoxy-3-methyl-2-[2-[[(3R)-1-methyl-3-piperidyl]amino]oxazolo[4,5-b]pyridin-5-yl]phenol;

and pharmaceutically acceptable salts thereof.

Most preferred examples of compounds of formula Ib as described herein are selected from
5-Chloro-2-[2-[[(3R)-1-ethyl-3-piperidyl]amino]oxazolo[4,5-b]pyridin-5-yl]-3-methyl-phenol
4-[2-[[(3R)-1-Ethyl-3-piperidyl]amino]oxazolo[4,5-b]pyridin-5-yl]-3-hydroxy-5-methyl-benzonitrile
4-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile and pharmaceutically acceptable salts thereof.
Particular examples of compounds of formula I as described herein are selected from (rac)-2-[2-[(1-Ethyl-3-piperidyl)amino]oxazolo[4,5-b]pyridin-5-yl]-3-methyl-5-(trifluoromethyl)phenol;
(rac)-2-[2-[3-[(Dimethylamino)methyl]morpholin-4-yl]oxazolo[4,5-b]pyridin-5-yl]-3-methyl-5-(trifluoromethyl)phenol;
2-[2-[(3R or 3S)-3-[(Dimethylamino)methyl]morpholin-4-yl]oxazolo[4,5-b]pyridin-5-yl]-3-methyl-5-(trifluoromethyl)phenol;
2-[2-[(3S or 3R)-3-[(Dimethylamino)methyl]morpholin-4-yl]oxazolo[4,5-b]pyridin-5-yl]-3-methyl-5-(trifluoromethyl)phenol;
(rac)-1-[5-[2-Hydroxy-6-methyl-4-(trifluoromethyl)phenyl]oxazolo[4,5-b]pyridin-2-yl]piperidin-3-ol;
(3R or 3S)-1-[5-[2-Hydroxy-6-methyl-4-(trifluoromethyl)phenyl]oxazolo[4,5-b]pyridin-2-yl]piperidin-3-ol;
(3S or 3R)-1-[5-[2-Hydroxy-6-methyl-4-(trifluoromethyl)phenyl]oxazolo[4,5-b]pyridin-2-yl]piperidin-3-ol;
3-Methyl-2-(2-morpholinooxazolo[4,5-b]pyridin-5-yl)-5-(trifluoromethyl)phenol;
(rac)-[2-[2-(Hydroxymethyl)morpholin-4-yl]oxazolo[4,5-b]pyridin-5-yl]-3-methyl-5-(trifluoromethyl)phenol;
3-Methyl-2-(2-piperazin-1-yloxazolo[4,5-b]pyridin-5-yl)-5-(trifluoromethyl)phenol;
3-Methyl-2-(2-piperazin-1-yloxazolo[4,5-b]pyridin-5-yl)-5-(trifluoromethyl)phenol formic acid salt;
3-Methyl-2-[2-[[3-(methylamino)cyclohexyl]amino]oxazolo[4,5-b]pyridin-5-yl]-5-(trifluoromethyl)phenol;
3-Methyl-2-(2-piperazin-1-yl-1H-imidazo[4,5-b]pyridin-5-yl)-5-(trifluoromethyl)phenol;
3-Methyl-2-(2-piperazin-1-yl-1H-imidazo[4,5-b]pyridin-5-yl)-5-(trifluoromethyl)phenol;2,2,2-trifluoroacetic acid;
(rac)-5-(5-(2-Hydroxy-6-methyl-4-(trifluoromethyl)phenyl)oxazolo[4,5-b]pyridin-2-yl) piperidin-2-one;
(rac)-3-Methyl-2-[2-[3-(methylamino)-1-piperidyl]oxazolo[4,5-b]pyridin-5-yl]-5-(trifluoromethyl) phenol;
(rac)-3-Methyl-2-[2-(tetrahydropyran-3-ylamino)oxazolo[4,5-b]pyridin-5-yl]-5-(trifluoromethyl)phenol;
1-[5-[2-Hydroxy-6-methyl-4-(trifluoromethyl)phenyl]oxazolo[4,5-b]pyridin-2-yl]azetidin-3-ol;
2-[2-[(3-Hydroxycyclohexyl)amino]oxazolo[4,5-b]pyridin-5-yl]-3-methyl-5-(trifluoromethyl)phenol;
2-[2-[[(3R)-1-Ethyl-3-piperidyl]amino]oxazolo[4,5-b]pyridin-5-yl]-3-methyl-5-(trifluoromethyl)phenol;
3-Methyl-2-[2-[[(3R)-1-methyl-3-piperidyl]amino]oxazolo[4,5-b]pyridin-5-yl]-5-(trifluoromethyl)phenol;
(rac)-3-Methyl-2-(2-(1-methylpiperidin-2-yl)-3H-imidazo[4,5-b]pyridin-5-yl)-5-(trifluoromethyl)phenol;
(rac)-3-Methyl-2-(2-(pyrrolidin-2-yl)-1H-imidazo[4,5-b]pyridin-5-yl)-5-(trifluoro-methyl)phenol;
(rac)-2-[2-[(1-Ethyl-3-piperidyl)amino]-1H-imidazo[4,5-b]pyridin-5-yl]-3-methyl-5-(trifluoromethyl)phenol;
2-[2-[[(3R or 3S)-1-Ethyl-3-piperidyl]amino]-1H-imidazo[4,5-b]pyridin-5-yl]-3-methyl-5-(trifluoromethyl)phenol;
2-[2-[[(3S or 3R)-1-Ethyl-3-piperidyl]amino]-1H-imidazo[4,5-b]pyridin-5-yl]-3-methyl-5-(trifluoromethyl)phenol;

and pharmaceutically acceptable salts thereof.

Preferred examples of compounds of formula I as described herein are selected from (rac)-2-[2-[(1-Ethyl-3-piperidyl)amino]oxazolo[4,5-b]pyridin-5-yl]-3-methyl-5-(trifluoromethyl)phenol;
2-[2-[(3S or 3R)-3-[(Dimethylamino)methyl]morpholin-4-yl]oxazolo[4,5-b]pyridin-5-yl]-3-methyl-5-(trifluoromethyl)phenol;
(3S or 3R)-1-[5-[2-Hydroxy-6-methyl-4-(trifluoromethyl)phenyl]oxazolo[4,5-b]pyridin-2-yl]piperidin-3-ol;
3-Methyl-2-(2-morpholinooxazolo[4,5-b]pyridin-5-yl)-5-(trifluoromethyl)phenol;
(rac)-[2-[2-(Hydroxymethyl)morpholin-4-yl]oxazolo[4,5-b]pyridin-5-yl]-3-methyl-5-(trifluoromethyl)phenol;
3-Methyl-2-(2-piperazin-1-yloxazolo[4,5-b]pyridin-5-yl)-5-(trifluoromethyl)phenol;
3-Methyl-2-(2-piperazin-1-yloxazolo[4,5-b]pyridin-5-yl)-5-(trifluoromethyl)phenol formic acid salt;
3-Methyl-2-[2-[[3-(methylamino)cyclohexyl]amino]oxazolo[4,5-b]pyridin-5-yl]-5-(trifluoromethyl)phenol;
(rac)-5-(5-(2-Hydroxy-6-methyl-4-(trifluoromethyl)phenyl)oxazolo[4,5-b]pyridin-2-yl) piperidin-2-one;
(rac)-3-Methyl-2-[2-[3-(methylamino)-1-piperidyl]oxazolo[4,5-b]pyridin-5-yl]-5-(trifluoromethyl) phenol;
2-[2-[[(3R)-1-Ethyl-3-piperidyl]amino]oxazolo[4,5-b]pyridin-5-yl]-3-methyl-5-(trifluoromethyl)phenol;
3-Methyl-2-[2-[[(3R)-1-methyl-3-piperidyl]amino]oxazolo[4,5-b]pyridin-5-yl]-5-(trifluoromethyl)phenol;
(rac)-3-Methyl-2-(2-(pyrrolidin-2-yl)-1H-imidazo[4,5-b]pyridin-5-yl)-5-(trifluoro-methyl)phenol;
2-[2-[[(3S or 3R)-1-Ethyl-3-piperidyl]amino]-1H-imidazo[4,5-b]pyridin-5-yl]-3-methyl-5-(trifluoromethyl)phenol;

and pharmaceutically acceptable salts thereof.

More preferred examples of compounds of formula I as described herein are selected from
2-[2-[(3S or 3R)-3-[(Dimethylamino)methyl]morpholin-4-yl]oxazolo[4,5-b]pyridin-5-yl]-3-methyl-5-(trifluoromethyl)phenol;
3-Methyl-2-(2-morpholinooxazolo[4,5-b]pyridin-5-yl)-5-(trifluoromethyl)phenol;
2-[2-[[(3R)-1-Ethyl-3-piperidyl]amino]oxazolo[4,5-b]pyridin-5-yl]-3-methyl-5-(trifluoromethyl)phenol;
3-Methyl-2-[2-[[(3R)-1-methyl-3-piperidyl]amino]oxazolo[4,5-b]pyridin-5-yl]-5-(trifluoromethyl)phenol;

and pharmaceutically acceptable salts thereof.

Most preferred examples of compounds of formula I as described herein are selected from
2-[2-[[(3R)-1-Ethyl-3-piperidyl]amino]oxazolo[4,5-b]pyridin-5-yl]-3-methyl-5-(trifluoromethyl)phenol;
3-Methyl-2-[2-[[(3R)-1-methyl-3-piperidyl]amino]oxazolo[4,5-b]pyridin-5-yl]-5-(trifluoromethyl)phenol;

and pharmaceutically acceptable salts thereof.

Processes for the manufacture of compounds of formula Ic as described herein are an object of the invention.

The present compounds of formula Ic and their pharmaceutically acceptable salts can be prepared by methods known in the art, for example, by processes described below which scheme 1 below comprises reacting a compound of formula II to a compound of formula Ic wherein R¹, R², R³, R⁸, R⁹, R^{X}, A¹, W and n are as described herein, A²=O or NH and R═H.

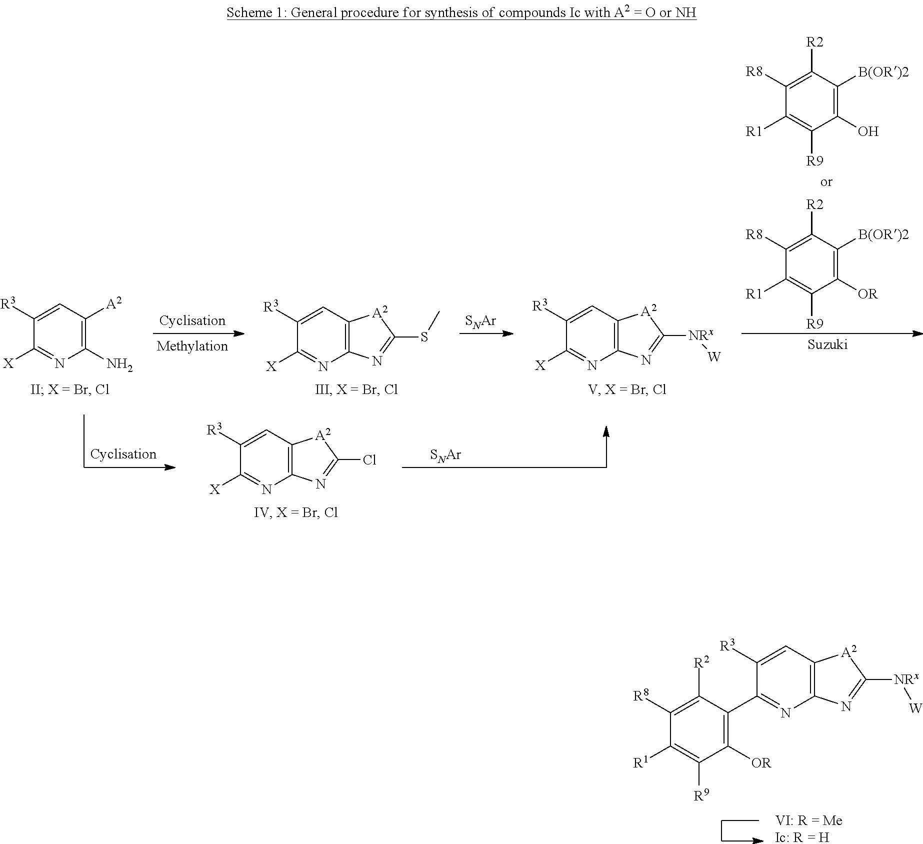

An alternative scheme to prepare compounds of formula Ic and their pharmaceutically acceptable salts is shown by the scheme 2 below which comprises reacting a compound of formula II to a compound of formula Ic wherein R¹, R², R³, R⁸, R⁹, R^{X}, A¹, A² and W are as described herein, and n is 0.

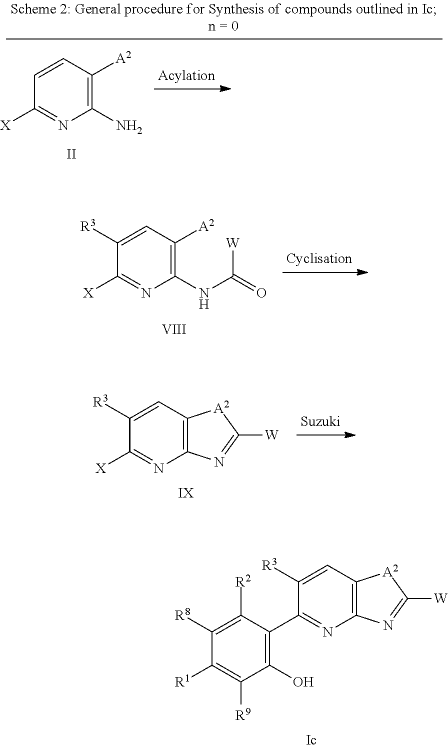

An alternative scheme to prepare compounds of formula Ic and their pharmaceutically acceptable salts is shown by the scheme 3 below which comprises reacting a compound of formula X to a compound of formula Ic wherein R¹, R², R³, R⁸, R⁹, R^{X}, n, and W are as described herein, A¹ is N and A² is O.

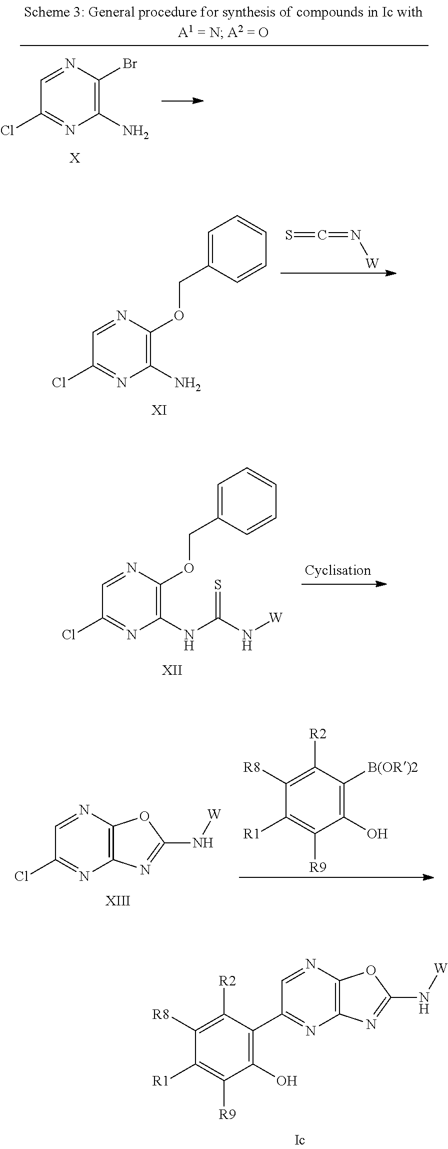

### General Synthesis Schemes

The compounds of formula Ic of the present invention may be prepared in accordance with the process variants described below and with the following schemes 1, 1a, 1b, 2, 3, 3a, 3b and 4. In addition, it is clear to the person skilled in the art that the order of transformations as described can be modified in various orders. These transformations can include e.g. the protection/cleavage of protecting groups, Pd-catalyzed couplings, reductive aminations and/or nucleophilic substitutions. The starting materials are commercially available or may be prepared in accordance with methods known in the public domain.

As depicted in scheme 1a, the commercially available building blocks of formula II wherein X is a halogen atom such as bromine, chlorine or iodine more preferably chlorine or bromine, can be submitted to a cyclisation commonly using 1,1′-Carbonyldiimidazole (CDI) or similar reagents as 1,1′-thiocarbonyldiimidazole or carbon disulfide followed by the addition of methyl iodide in the presence of an conventional inorganic base (such as potassium carbonate) or via reaction with a chlorinating agent such as POCl₃ to prepare compounds of general formula III or IV, respectively. These then can be subjected to a nucleophilic aromatic substitution in order to prepare compounds of formula V. The nucleophilic aromatic substitution are carried out with a suitable amine, e.g NR^{x}—W, but not limited to, wherein W and R^{x} have the meaning given in the claims of this current invention, with n being either 1 or 0, in the presence of organic bases as N,N-diisopropylethylamine (DIEA) or trimethylamine which are common and known to the skilled person and/or commercially available. Usually heating and 1,4-dioxane as solvent was used, but solvents such as dimethyl sulfoxide (DMSO), N,N-dimethyl-formamide (DMF) or N-methyl-2-pyrrolidine (NMP) in certain cases where higher temperature can be needed. Alternatively, the reaction can be also done under microwave irradiation. The left-hand side is added to the compound of general formula V to form the compound of formula VI using palladium catalyzed type reactions such as Suzuki cross coupling in the presence of a palladium catalyst and a boronic acid or boronic pinacol ester according to standard conditions well known to the skilled person, leading to final compounds of general formula Ic (depending of the substitution of the boronic acid or ester). In the case of a methyl ether-protecting group, in a final step, this is usually cleaved with boron tribromide (BBr₃) in dichloromethane delivering the compounds of general formula Ic. Specific examples are described in more detail for each exemplified compound below.

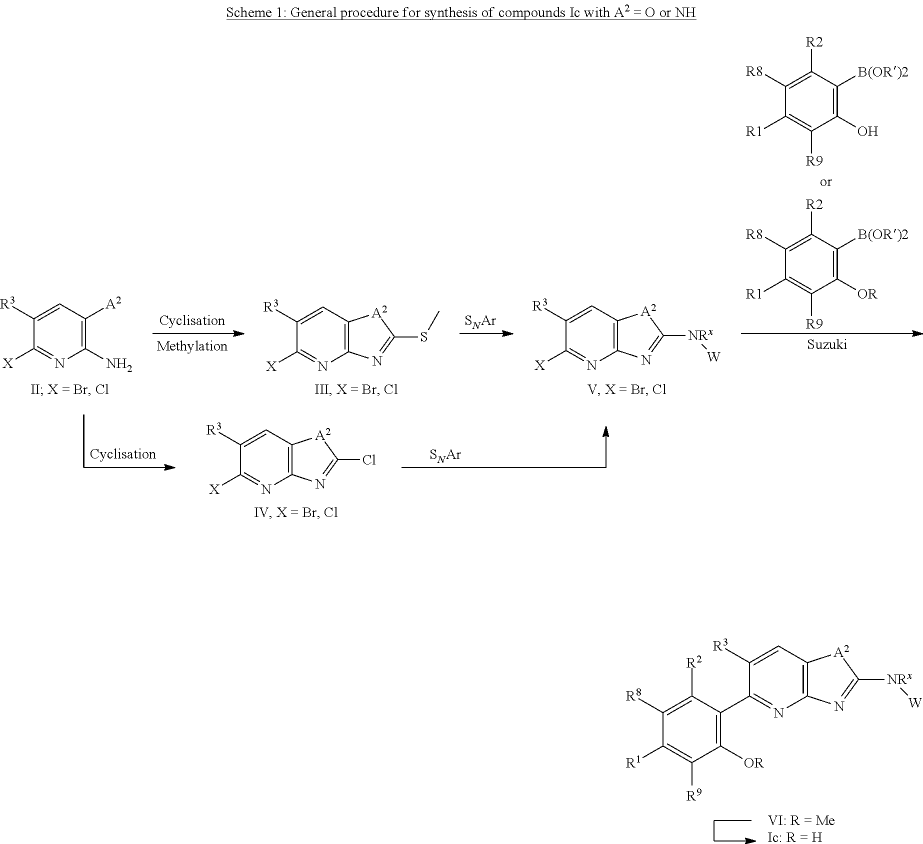

The synthesis of the compounds of formula Ic of the present invention are synthesized following to the general synthesis depicted in Scheme 1, wherein R′ can be OH or —C(CH₃)₂— and R is a protecting group known to the skilled person such as Me, SEM, benzyl or any other suitable protecting group for phenols (for examples, see in Protective Groups in organic Synthesis, T. W. Greene and P. G. M)

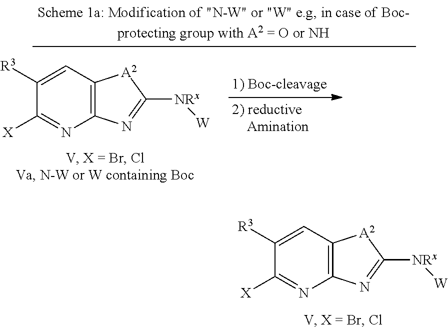

Further, in the cases where the amine NR^{x}—W or W contains e.g. a tert-butyloxycarbonyl (BOC) protecting group Va, an additional deprotection step was carried out either at an initial stage as described in Scheme 1a using TFA (trifluoroacetic acid) or HCl in dioxane, or at a later stage after introduction of the left-hand side aryl (Scheme 1a or 1b, VII). Afterwards, the respective amines were subjected to reductive aminations which are widely known to the skilled person, using an aldehyde or a ketone in the presence of a reducing agent such as borohydrides, more specifically e.g. sodium triacetoxyborohydride or sodium cyanoborohydride in the presence of an acid (e.g. acetic acid) or a buffer such as sodium acetate to yield wither derivatives of general formula V or final compounds Ic.

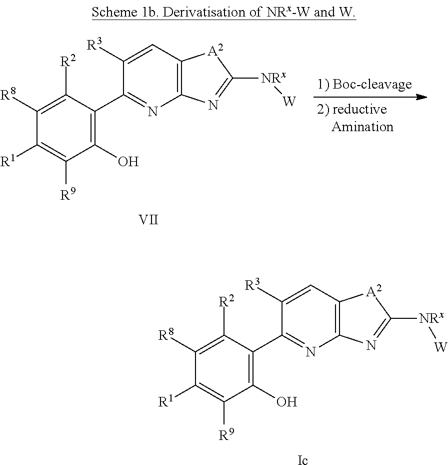

In case of n=0, commercially available building blocks of formula II wherein X is a halogen atom such as bromine, chlorine or iodine, more preferably chlorine or bromine, can be submitted to an acylation first under standard amide coupling conditions such as EDCI and HOBt, or HATU, or acyl chloride followed by cyclisation using either acidic conditions with e.g. polyphosphoric acid, Eaton's reagent or via Mitsunobu reaction, using DIAD and triphenylphosphine in THF or diethylether yielding the intermediate of general formula IX. Finally, Suzuki reaction as mentioned above, yields compounds of general formula Ic.

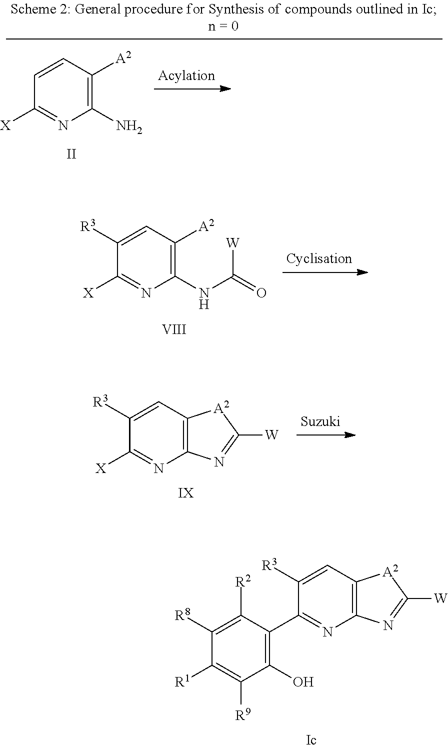

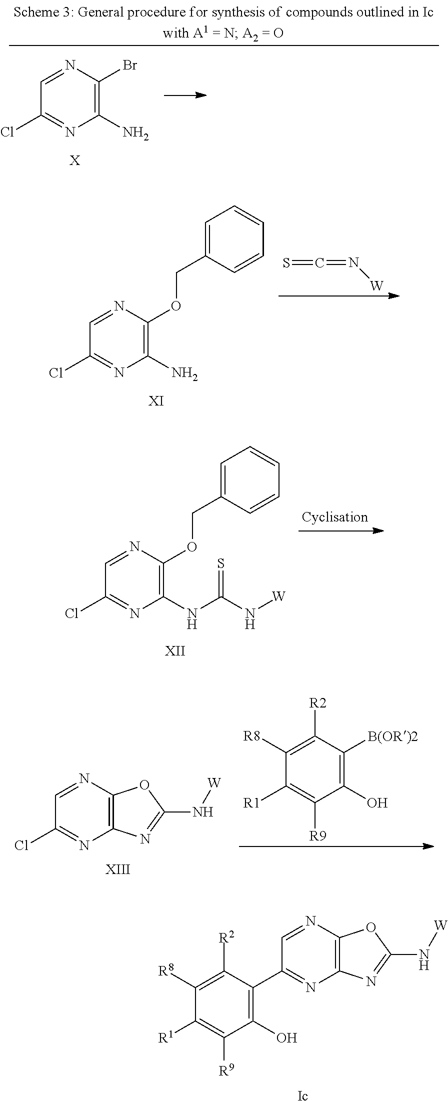

In case of A¹=N, commercially available building block of formula X, wherein chlorine and bromine can also be exchanged, can be subjected to reaction with benzyl alcohol in the presence of a strong base such as sodium hydride yielding compound of general formula XI. Suitable solvents are e.g. THF. In the next step, reaction with the corresponding isothiocyanate and a base, e.g. sodium hydride, gives compound of general formula XII. Cyclisation to intermediate XIII can be conducted by addition of reagents as e.g. tetrabutylazanium;iodide and hydrogen peroxide. In the final step, a Suzuki cross coupling in the presence of a palladium catalyst and a boronic acid or boronic pinacol ester according to standard conditions well known to the skilled person, leads to final compounds of general formula Ic.

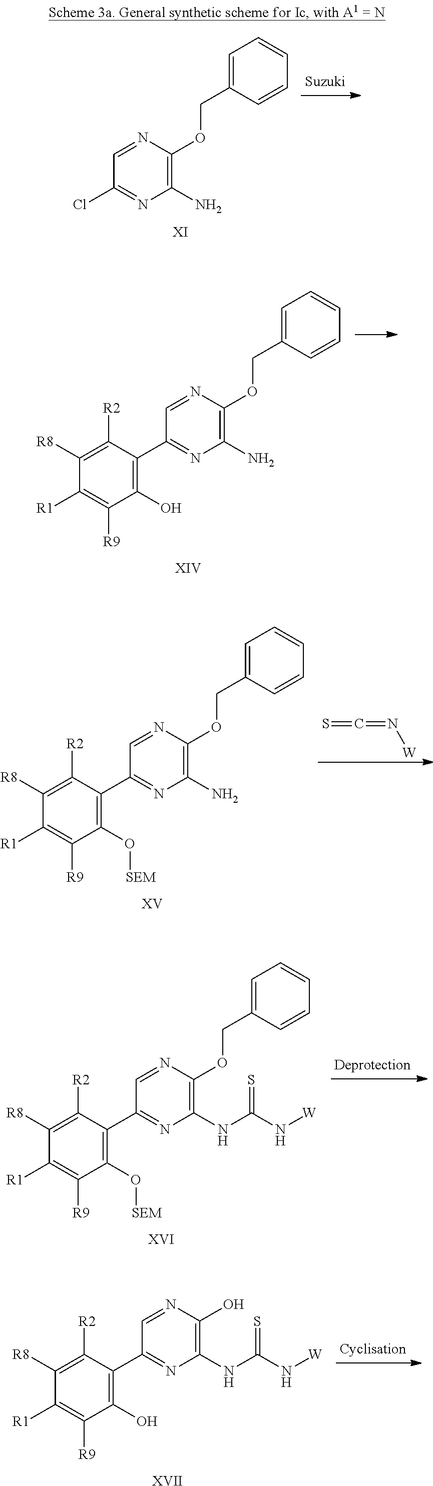

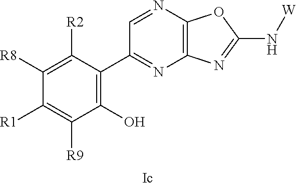

The compounds of general formula Ic, where A¹=N can also be obtained following the sequence outlined above. Starting from a similar precursor XI, Suzuki cross coupling in the presence of a palladium catalyst and a boronic acid or boronic pinacol ester according to standard conditions gives intermediate XIV. After protection of the phenol with e.g. SEM using standard procedures known to the person skilled in the art, compound XV was subjected to reaction with isothiocyanate in the presence of a base, e.g. sodium hydride, yielding intermediate XVI. Subsequent cleavage of the protecting group (SEM) under standard acidic conditions (e.g. TFA) gave access to XVII. In a final step, cyclisation was accomplished as outlined above.

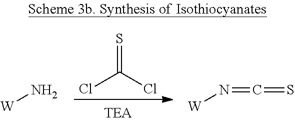

The isocyanates as describe can be prepared by reacting a primary amine of general formula as depicted in scheme 3b with thiophosgene under ice cooling. Suitable solvents for the reaction are e.g. DCM or DCE.

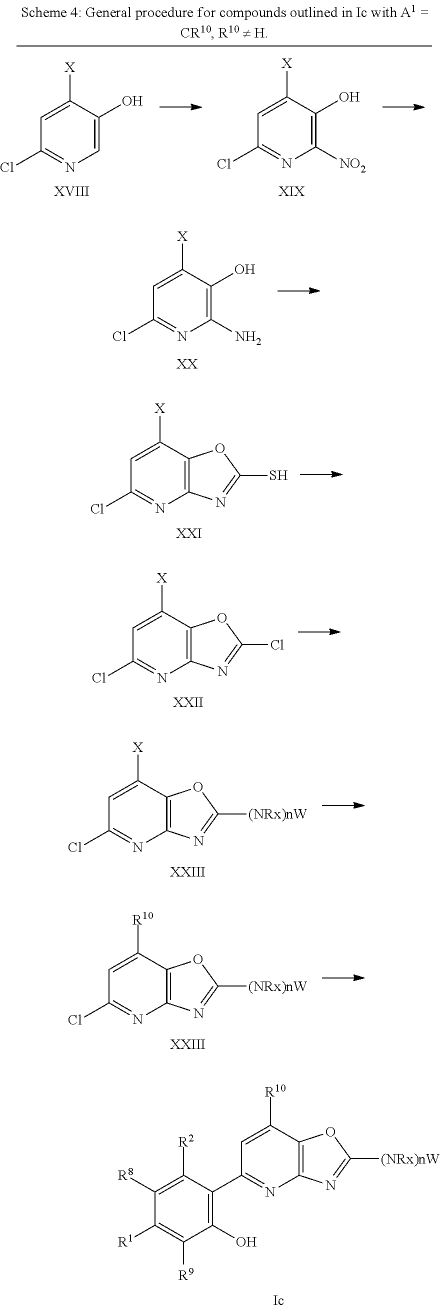

Starting from commercial building block XVIII, nitration was conducted in the presence of strong acids as eg. fuming nitric acid, known to the person skilled of the art, with X being iodine or bromine, preferably iodine. Reduction using tin(II)chloride or iron(III)chloride in e.g methanol afforded aniline XX. Subsequent cyclisation was achieved by addition of TCDI and heating. Key intermediate XXII was obtained after reaction with a chlorinating agent such as oxalyl chloride or POCl3 in, e.g. DMF at elevated temperatures. The compound was subjected to nucleophilic aromatic substitution, using a suitable amine NH—W or W containing a secondary amine or NR^{x}—W, wherein W and R^{x} have the meaning given for general formula Ic in the presence of organic bases as N,N-diisopropylethylamine (DIEA) or trimethylamine which are common and known to the skilled person and/or commercially available. Usually 1,4-dioxane as solvent was used, but solvents such as dimethyl sulfoxide (DMSO) or N-methyl-2-pyrrolidine (NMP) are also suitable. Different substituents as outlined in the claim for R¹⁰ and exemplified below were introduced with different methodologies, using palladium catalyzed coupling reactions, e.g. Buchwald reaction in the presence of an amide, carbonate or amine with respective ligands known to the skilled person, or Stille coupling using organotinn reagents and other suitable cross-coupling reactions. In a final step, Suzuki reaction was carried out to install the left hand side as depicted in general formula Ic.

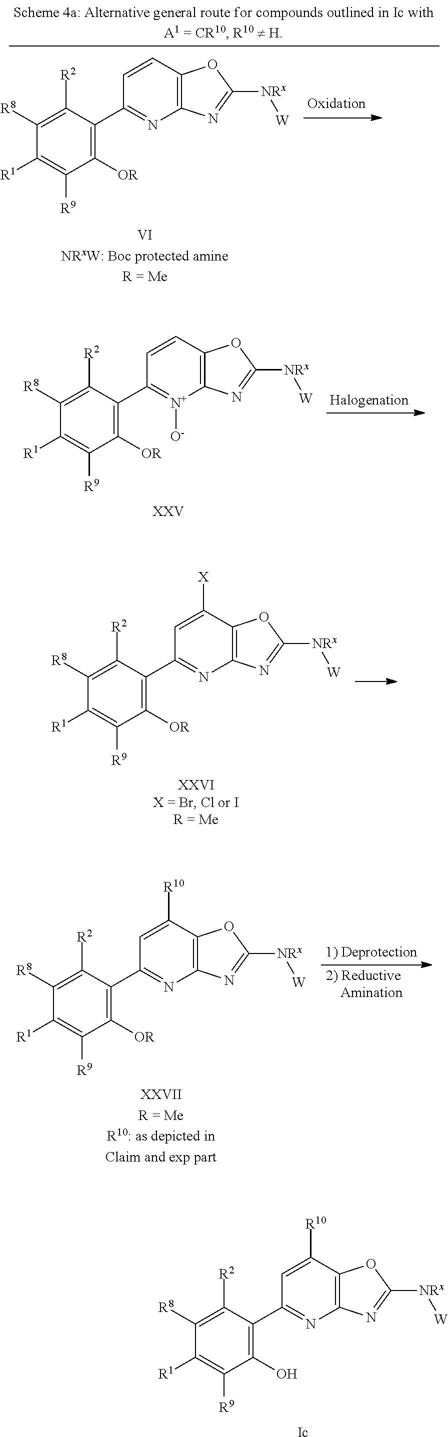

An alternative approach to modifications as described for R¹⁰ is outlined in Scheme 4a, starting from intermediate VI. Oxidation of the pyridine nitrogen was achieved in the presence of methyltrioxorhenium(vii) in the presence of hydrogen peroxide. Subsequent introduction of X═Cl or Br was obtained by the addition of suitable halogenation reagents such as e.g. oxalyl chloride in e.g. DMF. Introduction of R¹⁰ was achieved as outlined above using Pd-catalyzed cross coupling reactions or nucleophilic aromatic substitution. Finally, cleavage of the Boc-protected group under acidic conditions and reductive amination as outlined above gave final compounds Ic.

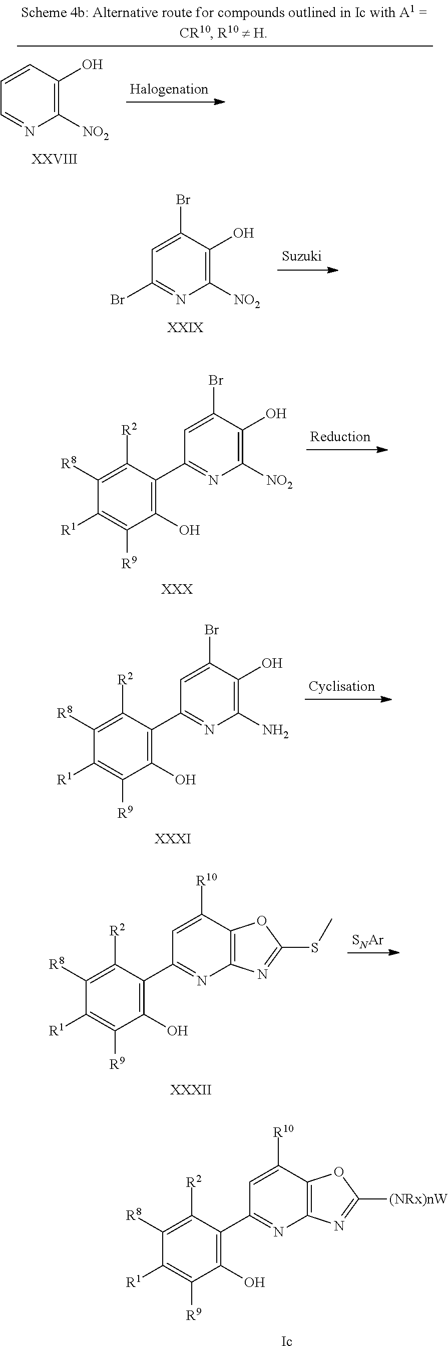

Starting from commercial building block XXVIII, halogenation was conducted in the presence of N-bromosuccinimide (NBS) or bromine in a solvent such as DMF, known to the person skilled of the art (XXIX). Subsequent Suzuki reaction with the corresponding boronic acid or boronic ester and reduction of the nitro-group using e.g. palladium on charcoal or, iron or iron(III)chloride in a suitable solvent such as methanol gives intermediate XXXI. Finally, cyclisation using TCDI followed by methylation with e.g. methyl iodide as described in detail above and subsequent nucleophilic aromatic substitution give compounds of general formula IC with R¹⁰H.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula Ib may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula Ib is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula Ic and their pharmaceutically acceptable salts can be processed with pharmaceutically inert, inorganic or organic adjuvants for the production of tablets, coated tablets, dragees, hard gelatin capsules, injection solutions or topical formulations Lactose, corn starch or derivatives thereof, talc, stearic acid or its salts etc. can be used, for example, as such adjuvants for tablets, dragees and hard gelatin capsules.

The compounds of formula Ib and their pharmaceutically acceptable salts can be processed with pharmaceutically inert, inorganic or organic adjuvants for the production of tablets, coated tablets, dragees, hard gelatin capsules, injection solutions or topical formulations Lactose, corn starch or derivatives thereof, talc, stearic acid or its salts etc. can be used, for example, as such adjuvants for tablets, dragees and hard gelatin capsules.

The compounds of formula I and their pharmaceutically acceptable salts can be processed with pharmaceutically inert, inorganic or organic adjuvants for the production of tablets, coated tablets, dragees, hard gelatin capsules, injection solutions or topical formulations Lactose, corn starch or derivatives thereof, talc, stearic acid or its salts etc. can be used, for example, as such adjuvants for tablets, dragees and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula Ic as described herein for use as a therapeutically active substance.

An embodiment of the present invention is a compound according to formula Ic as described herein for use in the treatment or prevention of a disease, disorder or condition, wherein the disease, disorder or condition is responsive to NLRP3 inhibition.

An embodiment of the present invention is a compound according to formula Ic as described herein for the treatment or prophylaxis of a disease, disorder or condition, wherein the disorder or condition is responsive to NLRP3 inhibition.

An embodiment of the present invention is a compound according to formula Ib as described herein for use as a therapeutically active substance.

An embodiment of the present invention is a compound according to formula Ib as described herein for use in the treatment or prevention of a disease, disorder or condition, wherein the disease, disorder or condition is responsive to NLRP3 inhibition.

An embodiment of the present invention is a compound according to formula Ib as described herein for the treatment or prophylaxis of a disease, disorder or condition, wherein the disorder or condition is responsive to NLRP3 inhibition.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia; (vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, Legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to formula Ic as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

An embodiment of the present invention is the use of a compound according to formula Ic as described herein in the treatment or prophylaxis of a disease, disorder or condition, wherein the disease, disorder or condition is responsive to NLRP3 inhibition.

An embodiment of the present invention is the use of a compound according to formula Ic as described herein in the treatment or prophylaxis of a disease, disorder or condition selected from Alzheimer's disease and Parkinson's disease.

An embodiment of the present invention is the use a compound according to formula Ic as described herein for use in the treatment or prophylaxis of a disease, disorder or condition selected from Asthma or COPD.

An embodiment of the present invention is the use a compound according to formula Ic as described herein for use in the treatment or prophylaxis of a disease, disorder or condition selected from inflammatory bowel disease (including Crohn's disease and ulcerative colitis).

An embodiment of the present invention is a compound according to formula Ic as described herein for the treatment or prophylaxis of a disease, disorder or condition selected from Alzheimer's disease and Parkinson's disease.

An embodiment of the present invention is a compound according to formula Ic as described herein for the treatment or prophylaxis of a disease, disorder or condition selected from Asthma or COPD.

An embodiment of the present invention is a compound according to formula Ic as described herein for the treatment or prophylaxis of a disease, disorder or condition selected from inflammatory bowel disease (including Crohn's disease and ulcerative colitis).

An embodiment of the present invention is the use of a compound according to formula Ic as described herein for preparation of a medicament for the treatment or prophylaxis of a disease, disorder or condition selected from Alzheimer's disease and Parkinson's disease.

An embodiment of the present invention is the use of a compound according to formula Ic as described herein for the preparation of a medicament for the treatment or prophylaxis of a disease, disorder or condition selected from Asthma or COPD.

An embodiment of the present invention is the use of a compound according to formula Ic as described herein for the preparation of a medicament for the treatment or prophylaxis of a disease, disorder or condition selected from inflammatory bowel disease (including Crohn's disease and ulcerative colitis).

An embodiment of the present invention is a method of treatment or prophylaxis of a disease, disorder or condition selected from Alzheimer's disease and Parkinson's disease, which method comprises administering an effective amount of a compound according to formula Ic as described herein.

An embodiment of the present invention is a method of treatment or prophylaxis of a disease, disorder or condition selected from Asthma or COPD, which method comprises administering an effective amount of a compound according to formula Ic as described herein.

An embodiment of the present invention is a method of treatment or prophylaxis of a disease, disorder or condition selected from inflammatory bowel disease (including Crohn's disease and ulcerative colitis), which method comprises administering an effective amount of a compound according to formula Ic as described herein.

An embodiment of the present invention relates to a method of inhibiting NLRP3, which method comprises administering an effective amount of a compound according to formula Ic as described herein.

Also an embodiment of the present invention are compounds of formula Ic as described herein, when manufactured according to any one of the described processes.

An embodiment of the present invention is a pharmaceutical composition comprising a compound according to formula Ic as described herein and a therapeutically inert carrier.

An embodiment of the present invention is the use of a compound according to formula Ib as described herein in the treatment or prophylaxis of a disease, disorder or condition, wherein the disease, disorder or condition is responsive to NLRP3 inhibition.

An embodiment of the present invention is the use of a compound according to formula Ib as described herein in the treatment or prophylaxis of a disease, disorder or condition selected from Alzheimer's disease and Parkinson's disease.

An embodiment of the present invention is the use a compound according to formula Ib as described herein for use in the treatment or prophylaxis of a disease, disorder or condition selected from Asthma or COPD.

An embodiment of the present invention is the use a compound according to formula Ib as described herein for use in the treatment or prophylaxis of a disease, disorder or condition selected from inflammatory bowel disease (including Crohn's disease and ulcerative colitis).

An embodiment of the present invention is a compound according to formula Ib as described herein for the treatment or prophylaxis of a disease, disorder or condition selected from Alzheimer's disease and Parkinson's disease.

An embodiment of the present invention is a compound according to formula Ib as described herein for the treatment or prophylaxis of a disease, disorder or condition selected from Asthma or COPD.

An embodiment of the present invention is a compound according to formula Ib as described herein for the treatment or prophylaxis of a disease, disorder or condition selected from inflammatory bowel disease (including Crohn's disease and ulcerative colitis).

An embodiment of the present invention is the use of a compound according to formula Ib as described herein for preparation of a medicament for the treatment or prophylaxis of a disease, disorder or condition selected from Alzheimer's disease and Parkinson's disease.

An embodiment of the present invention is the use of a compound according to formula Ib as described herein for the preparation of a medicament for the treatment or prophylaxis of a disease, disorder or condition selected from Asthma or COPD.

An embodiment of the present invention is the use of a compound according to formula Ib as described herein for the preparation of a medicament for the treatment or prophylaxis of a disease, disorder or condition selected from inflammatory bowel disease (including Crohn's disease and ulcerative colitis).

An embodiment of the present invention is a method of treatment or prophylaxis of a disease, disorder or condition selected from Alzheimer's disease and Parkinson's disease, which method comprises administering an effective amount of a compound according to formula Ib as described herein.

An embodiment of the present invention is a method of treatment or prophylaxis of a disease, disorder or condition selected from Asthma or COPD, which method comprises administering an effective amount of a compound according to formula Ib as described herein.

An embodiment of the present invention is a method of treatment or prophylaxis of a disease, disorder or condition selected from inflammatory bowel disease (including Crohn's disease and ulcerative colitis), which method comprises administering an effective amount of a compound according to formula Ib as described herein.

An embodiment of the present invention relates to a method of inhibiting NLRP3, which method comprises administering an effective amount of a compound according to formula Ib as described herein.

Also an embodiment of the present invention are compounds of formula Ib as described herein, when manufactured according to any one of the described processes.

An embodiment of the present invention is a pharmaceutical composition comprising a compound according to formula Ib as described herein and a therapeutically inert carrier.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use of a compound according to formula I as described herein in the treatment or prophylaxis of a disease, disorder or condition selected from Alzheimer's disease and Parkinson's disease.

An embodiment of the present invention is the use a compound according to formula I as described herein for use in the treatment or prophylaxis of a disease, disorder or condition selected from Asthma or COPD.

An embodiment of the present invention is the use a compound according to formula I as described herein for use in the treatment or prophylaxis of a disease, disorder or condition selected from inflammatory bowel disease (including Crohn's disease and ulcerative colitis).

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Asthma or COPD.

An embodiment of the present invention is a compound according to formula I as described herein for the treatment or prophylaxis of a disease, disorder or condition selected from inflammatory bowel disease (including Crohn's disease and ulcerative colitis).

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for the preparation of a medicament for the treatment or prophylaxis of a disease, disorder or condition selected from Asthma or COPD.

An embodiment of the present invention is the use of a compound according to formula I as described herein for the preparation of a medicament for the treatment or prophylaxis of a disease, disorder or condition selected from inflammatory bowel disease (including Crohn's disease and ulcerative colitis).

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Asthma or COPD, which method comprises administering an effective amount of a compound according to formula I as described herein.

An embodiment of the present invention is a method of treatment or prophylaxis of a disease, disorder or condition selected from inflammatory bowel disease (including Crohn's disease and ulcerative colitis), which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS).
The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)
   The results of the pyroptosis assay are summarised in Table 1 below as THP IC₅₀.

### Human Whole Blood IL-13 Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.
Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.10% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

TABLE 1
NLRP3 inhibitory activity

| Example No. | THP-1 pyroptosis Assay IC₅₀ (nM) |
|---|---|
| 1 | 1.9 |
| 2 | 17.4 |
| 2A | 128.6 |
| 2B | 16.9 |
| 3 | 75.4 |
| 3A | 75.0 |
| 3B | 28.6 |
| 4 | 29.4 |
| 5 | 31.9 |
| 6 | 86.6 |
| 7 | 82.1 |
| 8 | 302.0 |
| 9 | 15.3 |
| 10 | 107.4 |
| 11 | 216.6 |
| 12 | 214.7 |
| 13 | 441.6 |
| 14 | 1.0 |
| 15 | 0.7 |
| 16 | 330.0 |
| 17 | 183.0 |
| 18A | 4.1 |
| 18B | 392.4 |
| 19 | 0.4 |
| 20 | 1.2 |
| 20A | 148.5 |
| 20B | 0.5 |
| 21 | 0.6 |
| 22 | 5.4 |
| 23 | 4.2 |
| 24 | 1.7 |
| 25 | 1.6 |
| 26 | 2.8 |
| 27A | 578 |
| 27B | 2.7 |
| 28 | 4.6 |
| 29 | 5.1 |
| 30 | 4.5 |
| 31 | 4.0 |
| 32 | 3.0 |
| 33 | 5.8 |
| 34 | 18.9 |
| 35 | 27.3 |
| 36 | 32.0 |
| 37B | 1.1 |
| 37A | 216.2 |
| 38 | 85.3 |
| 39 | 1.9 |
| 40 | 27.4 |
| 41 | 0.6 |
| 42 | 5.1 |
| 43 | 30.5 |
| 44 | 0.7 |
| 45 | 0.9 |
| 46 | 1.0 |
| 47 | 1.2 |
| 48 | 1.2 |
| 49 | 1.6 |
| 50 | 1.7 |
| 51 | 1.8 |
| 52 | 2.3 |
| 53 | 2.6 |
| 54 | 10.1 |
| 55 | 17.8 |
| 56 | 18.9 |
| 57 | 50.3 |
| 58 | 95.9 |
| 59 | 30.1 |
| 60 | 17.9 |
| 61 | 39.4 |
| 62 | 84.8 |
| 63 | 57.5 |
| 64 | 4.9 |
| 64B | 13.9 |
| 64A | 1.7 |
| 65 | 20.3 |
| 66B | 256.2 |
| 66A | 397.5 |
| 66 | 148.6 |
| 67 | 290.0 |
| 68 | 62.4 |
| 69 | 2.7 |
| 70 | 3.8 |
| 71 | 1.1 |
| 72A | 1.0 |
| 72B | 309.7 |
| 73 | 30.0 |
| 73B | 78.7 |
| 73A | 18.7 |
| 74 | 9.2 |
| 75 | 20.8 |
| 76 | 143.0 |
| 77 | 134.7 |
| 78 | 58.3 |
| 79 | 94.9 |
| 80 | 109.9 |
| 81 | 72.3 |
| 82 | 128.2 |
| 83 | 5.1 |
| 84 | 0.8 |
| 85 | 15.4 |
| 86 | 0.6 |
| 87 | 75.6 |
| 88 | 6.0 |
| 89 | 536.1 |
| 90 | 22.6 |
| 91 | 4.9 |
| 92 | 26.9 |
| 93 | 2.1 |
| 94 | 11.9 |
| 95 | 11.2 |
| 96 | 56.1 |
| 97 | 93.9 |
| 98 | 11.1 |
| 99A | 1.9 |
| 99B | 380->1000 |
| 100 | 18.4 |
| 101 | 6.0 |
| 102 | 6.5 |
| 103 | 4.6 |
| 104 | 1.9 |
| 105 | 8.9 |
| 106 | 22.0 |
| 107 | 8.8 |
| 108 | 16.8 |
| 109 | 1.4 |
| 110 | 5.8 |
| 111 | 2.2 |
| 112 | 6.6 |
| 113 | 0.5 |
| 114 | 1.1 |
| 115 | 48.5 |
| 116 | 6.9 |
| 117 | 5.2 |
| 118A | 50.8 |
| 118B | 30.6 |
| 119 | 23.0 |
| 120 | 107.7 |
| 121 | 2.7 |
| 122 | 1.0 |
| 123 | 0.7 |
| 124 | 53.8 |
| 125 | 3.0 |
| 126 | 3.9 |
| 127 | 20.6 |
| 128 | 1.8 |
| 129 | 1.7 |
| 130 | 1.9 |
| 131 | 9.6 |
| 132 | 0.7 |
| 133 | 80.3 |
| 134 | 1.9 |
| 135 | 505.0 |
| 136 | 1.0 |
| 137 | 4.8 |
| 138 | 233.6 |
| 139B | 59.9 |
| 139A | 3.7 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization. For some examples, e.g. 99a and 99b, the absolute stereoconfiguration was not determined but attributed based on biological activity (determined e.g. in the THP-assay).

### EXPERIMENTAL METHODS

### Abbreviations

| | |
|---|---|
| aq | aqueous |
| DCM | Dichloromethane |
| DIPEA | diisopropylethylamine |
| DMF | N,N-Dimethylformamide |
| hr(s), h(s) | hour(s) |
| HOBt | hydroxybenzotriazole |
| EDCI | 1-ethyl-3-(3- |
| | dimethylaminopropyl)carbodiimide |
| Methanesulfonato(2- | XPhos Pd G3 |
| dicyclohexylphosphino-2′,4′,6′-tri-i- | |
| propyl-1,1′-biphenyl)(2′-amino-1,1′- | |
| biphenyl-2-yl)palladium(ii) | |
| RT, rt, r.t. | Room temperature |
| tBME, MTBE | methyl tert-butyl ether |
| Prep-HPLC | preparative-High performance |
| | liquid chromatography |
| TFA | trifluoroacetic acid |
| T₃P | propylphosphonic anhydride |
| MeCN | acetonitrile |
| sat | saturated |

### Analytical Methods

NMR spectra were run on Bruker 400 MHz spectrometers using ICON-NMR, under TopSpin program control. Spectra were measured at 298 K, unless indicated otherwise, and were referenced relative to the solvent resonance.

### LC-MS Methods:

Using SHIMADZU LCMS-2020, Agilent 1200 LC/G1956A MSD and Agilent 1200\G6110A, Agilent 1200 LC & Agilent 6110 MSD. Mobile Phase: A: 0.025% NH3·H2O in water (v/v); B: Acetonitrile. Column: Kinetex EVO C18 2.1×30 mm, 5 um.

### Systems

### Waters Acquity UPLC

Binary Pump
Autosampler Waters 2777C (alias CTC Pal HT)
Column Managers (4 Columns)
Photodiode Array Detector (PDA)
Single Quadrupole Mass Spectrometer (SQD1 respectively SQD2)

### Eluents

Channel A: Water 0.10% Formic Acid
Channel B: Acetonitrile 0.07% Formic Acid

### Built-In Columns (@ 50° C.):

Column 1: Agilent Zorbax Eclipse Plus C18, Rapid Resolution HT, 2.1×30 mm, 1.8 μm, Part. no. 959731-902
Column 2: (only for MS1+5+7): Waters Acquity UPLC BEH C18, 2.1×50 mm, 1.7 μm, Part. no. 186002350
Column 3: none
Column 4: none (Flow injection)

### Methods:

Fast_Gradient (2 min, Column 1, Mass range m/z 150-900)

| Time (min) | Flow Rate (ml/min) | % A | % B |
|---|---|---|---|
| Initial | 0.8 | 97 | 3 |
| 0.2 | 1.0 | 97 | 3 |
| 1.7 | 1.0 | 3 | 97 |
| 2.0 | 1.0 | 3 | 97 |
| 2.1 | 1.0 | 97 | 3 |

### Purification Method (Step B)

Automated reversed phase column chromatography was carried out using a Gilson GX-281 system driven by a Gilson-322 pump module, Gilson-156 UV photometer detection unit and Gilson-281 fraction collector.
Waters Xbridge: 150*25 mm*5 um
pH (water(10 mM NH₃H2O)-ACN) 7-8
Average particle size: 5 μm
The column was conditioned before use with 100% MeCN (2 min) then brought to 1% MeCN (in 0.8 min). Flow rate=25 m/min.

### Separation Runs:

| Time (min) | A: Water (10 mM NH₃H₂O) | B MeCN |
|---|---|---|
| 0 | 82% | 18% |
| 1.0 | 82% | 18% |
| 10.0 | 52% | 48% |
| 10.2 | 0% | 100% |
| 12.0 | 0% | 100% |
| 12.2 | 95% | 5% |
| 13.0 | 95% | 5% |

Detection wavelength: 220 and 254 nm. Before each new run, the cartridge was cleaned using the conditioning method.

### General Procedures

### GP1: Aromatic Nucleophilic Substitution

To a mixture of 5-chloro-2-(methylthio)oxazolo[4,5-b]pyridine (CAS #1783370-92-2, 1.00 eq) in 1,4-dioxane (0.5 mmol/mL) was added the corresponding amine (1.16 eq) followed by addition of triethylamine (1.14 eq). The solution was stirred at 90° C. for 16 hrs. After cooling to r.t. the mixture was extracted with a suitable organic solvent such as ethyl acetate and the organic phase washed with e.g. saturated aq. NaHCO₃-solution. The combined organic layers were dried e.g. over sodium sulfate, filtered and concentrated in vacuo and finally purified either via column chromatography or HPLC.

### GP2: Suzuki

### 2a: With Dppf

A mixture of 5-chloro-N-[(3R)-1-ethyl-3-piperidyl]oxazolo[4,5-b]pyridin-2-amine (1.0 eq), and the corresponding boronic acid or boronate ester (1.4-1.7 eq), potassium carbonate (4.8 eq) and 1,1′-bis(diphenylphosphino)ferrocene-palladium(ii) dichloride dichloromethane complex (0.15 eq) were dissolved in 1,4-dioxane (0.1067 mmol/mL) and water (0.1067 mmol/mL, v/v 2/1) and the mixture was flushed with argon and stirred at 95° C. for consumption of the starting material (mostly 8-16 h). After cooling to r.t. the mixture was extracted with a suitable organic solvent such as ethyl acetate and the organic phase washed with e.g. half-saturated aq. NH4Cl-solution. The combined organic layers were dried e.g. over sodium sulfate, filtered and concentrated in vacuo and finally purified either via column chromatography or HPLC.

2b. With Xphos

A mixture of 5-chloro-N-[(3R)-1-ethyl-3-piperidyl]oxazolo[4,5-b]pyridin-2-amine (1.00 eq), and the corresponding boronic acid or boronate ester (1.4-1.7 eq), cesium carbonate (3.0 eq) and XPhos Pd G3 (0.1 eq) were dissolved in 1,4-dioxane and water (v/v 4/1) and the mixture was flushed with argon and stirred at 90° C. until consumption of starting material. After cooling to r.t. the mixture was extracted with a suitable organic solvent such as ethyl acetate and the organic phase washed with e.g. brine. The combined organic layers were dried e.g. over sodium sulfate, filtered and concentrated in vacuo and finally purified either via column chromatography or HPLC.

GP3: Boc Deprotection with TFA

The corresponding carboxylic acid tert-butyl ester (1.0 eq) was treated with TFA (9.0 eq) in dichloromethane and the reaction was stirred at r.t. until starting material was consumed. The solvent was then removed under reduced pressure.

### GP4: Reductive Amination

To a suspension of the corresponding primary or secondary amine (free base; HCl or TFA salt) (1.0 eq) in dry DCM (0.0702 mmol/mL) was added acetaldehyde (2.5 eq) followed by sodium acetate (2.5 eq) under ice-bath cooling. Then, sodium triacetoxyborohydride (1.8 eq) was added at 0° C. The reaction mixture was stirred at stirred at 0° C. for 5 min, then the ice bath was removed and stirred for 3-5 hours at 23° C. Then, the reaction mixture was carefully basified with e.g. aq. NaHCO₃ solution under ice cooling, and then extracted with a suitable organic solvent such as dichloromethane. The combined organic layers were dried over e.g. sodium sulfate, filtered and concentrated in vacuo and finally purified via either column chromatography or HPLC.

In certain cases a mixture of DCM/MeOH was used as solvent.

### EXAMPLES

All examples and intermediates were prepared under nitrogen atmosphere if not specified otherwise.

### Example 1

### (rac)-2-[2-[(1-Ethyl-3-piperidyl)amino]oxazolo[4,5-b]pyridin-5-yl]-3-methyl-5-(trifluoromethyl)phenol

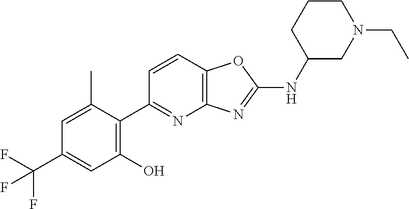 [SMILES: CCN1CCCC(Nc2nc3nc(-c4c(C)cc(C(F)(F)F)cc4O)ccc3o2)C1]

### Step 1: (rac)-5-Chloro-N-(1-ethyl-3-piperidyl)oxazolo[4,5-b]pyridin-2-amine

To a mixture of 5-chloro-2-(methylthio)oxazolo[4,5-b]pyridine (CAS #1783370-92-2, 80 mg, 0.379 mmol, 1 eq) in 1,4-dioxane (0.80 mL) was added (rac)-(1-ethyl-3-piperidyl)amine (CAS #6789-94-2, 0.061 mL, 0.427 mmol, 1.13 eq) followed by triethylamine (0.060 mL, 0.430 mmol, 1.14 eq). The brown solution was stirred at 90° C. for 16 hrs. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was purified by flash chromatography (silica gel, 12 g, gradient 0% to 10% methanol in dichloromethane) to afford the title compound (96 mg, 86% yield) as a light yellow oil. LCMS: m/z 281.3 [M+H]+, ESI pos.

### Step 2: (rac)-2-[2-[(1-Ethyl-3-piperidyl)amino]oxazolo[4,5-b]pyridin-5-yl]-3-methyl-5-(trifluoromethyl)phenol

A mixture of (rac)-5-chloro-N-(1-ethyl-3-piperidyl)oxazolo[4,5-b]pyridin-2-amine (Example 1, step 1) (92 mg, 0.311 mmol, 1.0 eq), 3-methyl-2-(4,4,5,5-tetramethyl-1,3,2-dioxaborolan-2-yl)-5-(trifluoromethyl)phenol (CAS #2557358-38-8, 136 mg, 0.450 mmol, 1.45 eq), XPhos Pd G3 (27 mg, 0.032 mmol, 0.102 eq) and cesium carbonate (305 mg, 0.936 mmol, 3.01 eq) in 1,4-dioxane (1.2 mL) and water (0.30 mL) was flushed with argon and stirred at 100° C. for 16 hrs. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined to afford the title compound (82 mg, 60% yield) as a light yellow solid. LCMS: m/z 421.3 [M+H]+, ESI pos.

### Example 2, 2A and 2B

### (rac)-2-[2-[3-[(Dimethylamino)methyl]morpholin-4-yl]oxazolo[4,5-b]pyridin-5-yl]-3-methyl-5-(trifluoromethyl)phenol and respective enantiomers 2A and 2B

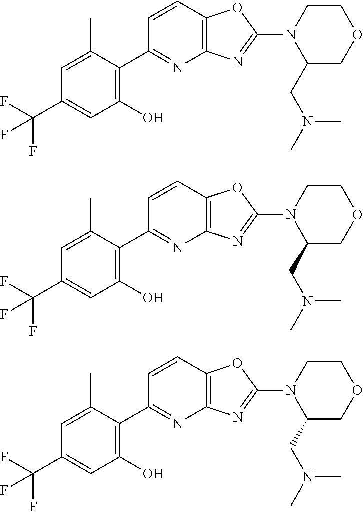

### Step 1: (rac)-1-[4-(5-Chlorooxazolo[4,5-b]pyridin-2-yl)morpholin-3-yl]-N,N-dimethyl-methanamine

To a mixture of 5-chloro-2-(methylthio)oxazolo[4,5-b]pyridine (CAS #1783370-92-2, 80 mg, 0.379 mmol, 1.0 eq) in (rac)-N-methyl-2-pyrrolidinone (0.720 mL) was added dimethyl(morpholin-3-ylmethyl)amine (CAS #128454-20-6, 61 mg, 0.423 mmol, 1.12 eq) followed by triethylamine (0.060 mL, 0.430 mmol, 1.14 eq). The brown solution was stirred at 150° C. for 16 hrs. The reaction mixture was cooled to room temperature and extracted with ethyl acetate and water. The aqueous layer was backextracted with ethyl acetate. The organic layers were washed four times with water and once with brine. The combined organic layers were dried over sodium sulfate, filtered and concentrated in vacuo. The crude product was purified by flash chromatography (silica gel, 12 g, gradient 0% to 5% methanol in dichloromethane) to afford the title compound (85 mg, 72% yield) as a light brown oil. LCMS: m/z 297.2 [M+H]+, ESI pos.

### Step 2: (rac)-2-[2-[3-[(Dimethylamino)methyl]morpholin-4-yl]oxazolo[4,5-b]pyridin-5-yl-3-methyl-5-(trifluoromethyl)phenol

A mixture of (rac)-1-[4-(5-chlorooxazolo[4,5-b]pyridin-2-yl)morpholin-3-yl]-N,N-dimethyl-methanamine (Example 2, step 1) (79 mg, 0.253 mmol, 1 eq), 3-methyl-2-(4,4,5,5-tetramethyl-1,3,2-dioxaborolan-2-yl)-5-(trifluoromethyl)phenol (CAS #2557358-38-8, 111 mg, 0.367 mmol, 1.45 eq), cesium carbonate (237 mg, 0.727 mmol, 2.88 eq) and XPhos Pd G3 (21 mg, 0.025 mmol, 0.098 eq) in 1,4-dioxane (1.0 mL) and water (0.250 mL) was flushed with argon and stirred at 100° C. for 16 hrs. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane). All fractions containing product were combined to afford the title compound (93 mg, 80% yield) as a light yellow foam. LCMS: m/z 437.4 [M+H]⁺, ESI pos.

### Step 3: 2-[2-[(3R or 3S)-3-[(Dimethylamino)methyl]morpholin-4-yl]oxazolo[4,5-b]pyridin-5-yl]-3-methyl-5-(trifluoromethyl)phenol and 2-[2-[(3S or 3R)-3-[(Dimethylamino)methyl]morpholin-4-yl]oxazolo[4,5-b]pyridin-5-yl]-3-methyl-5-(trifluoromethyl)phenol

Racemate (rac)-2-[2-[3-[(Dimethylamino)methyl]morpholin-4-yl]oxazolo[4,5-b]pyridin-5-yl]-3-methyl-5-(trifluoromethyl)phenol (Example 2, step 2) (60 mg, 0.131 mmol, 1 eq) was separated by chiral SFC (column: Chiralpak IC, eluent B: 15% methanol+0.2% diethylamine) to afford the two enantiomers example 2A (first eluting, Rt=1.51 min) (23 mg, 38% yield) as a light brown foam; m/z 437.3 [M+H]+, ESI pos and example 2B (second eluting, Rt=1.74 min) (27 mg, 45% yield) as a light brown foam. LCMS: m/z 437.3 [M+H]+, ESI pos.

### Examples 3, 3A and 3B

### (rac)-1-[5-[2-Hydroxy-6-methyl-4-(trifluoromethyl)phenyl]oxazolo[4,5-b]pyridin-2-yl]piperidin-3-ol and respective enantiomers 3A and 3B

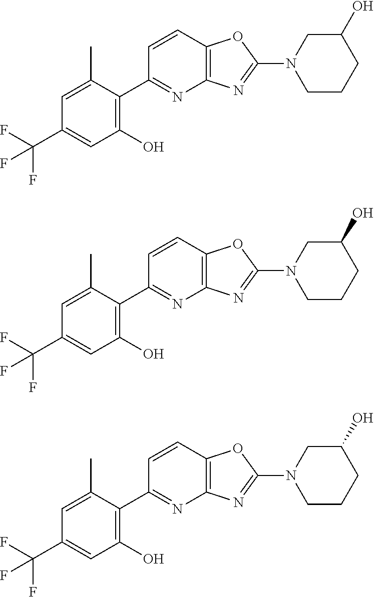 [SMILES: Cc1cc(C(F)(F)F)cc(O)c1-c1ccc2oc(N3CCCC(O)C3)nc2n1 ; Cc1cc(C(F)(F)F)cc(O)c1-c1ccc2oc(N3CCC[C@H](O)C3)nc2n1 ; Cc1cc(C(F)(F)F)cc(O)c1-c1ccc2oc(N3CCC[C@@H](O)C3)nc2n1]

### Step 1: (rac)-1-(5-Chlorooxazolo[4,5-b]pyridin-2-yl)piperidin-3-ol

A mixture of 5-chloro-2-(methylthio)oxazolo[4,5-b]pyridine (CAS #1783370-92-2, 100 mg, 0.473 mmol, 1.0 eq), piperidin-3-ol (CAS #6859-99-0, 53 mg, 0.524 mmol, 1.11 eq) and triethylamine (0.073 mL, 0.524 mmol, 1.1 eq) in 1,4-dioxane (1.0 mL) was stirred at 90° C. for 16 hrs. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 70% ethyl acetate in heptane) to afford the title compound (117 mg, 93% yield) as a white solid. LCMS: m/z 254.1 [M+H]⁺, ESI pos.

### Step 2: (rac)-1-[5-[2-Methoxy-6-methyl-4-(trifluoromethyl)phenyl]oxazolo[4,5-b]pyridin-2-yl]piperidin-3-ol

A mixture of (rac)-1-(5-chlorooxazolo[4,5-b]pyridin-2-yl)piperidin-3-ol (Example 3, step 1) (117 mg, 0.438 mmol, 1.0 eq), 2-[2-methoxy-6-methyl-4-(trifluoromethyl)phenyl]-4,4,5,5-tetramethyl-1,3,2-dioxaborolane (CAS #2557358-25-3, 194 mg, 0.614 mmol, 1.4 eq), potassium carbonate (305 mg, 2.21 mmol, 5.04 eq) and 1,1′-bis(diphenylphosphino)ferrocene-palladium(ii)dichloride dichloromethane complex (44 mg, 0.054 mmol, 0.123 eq) in 1,4-dioxane (2.8 mL) and water (1.4 mL) was flushed with argon and stirred at 110° C. for 4 hrs and at room temperature for 16 hrs. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound (194 mg, 98% yield, 90% purity) as a light brown waxy solid. LCMS: m/z 408.3 [M+H]+, ESI pos.

### Step 3: (rac)-1-[5-[2-Hydroxy-6-methyl-4-(trifluoromethyl)phenyl]oxazolo[4,5-b]pyridin-2-yl]piperidin-3-ol

To a solution of (rac)-1-[5-[2-methoxy-6-methyl-4-(trifluoromethyl)phenyl]oxazolo[4,5-b]pyridin-2-yl]piperidin-3-ol (Example 3, step 2) (194 mg, 0.429 mmol, 1.0 eq, 90% purity) in dichloromethane (0.800 mL) was added boron tribromide (1 M solution in dichloromethane) (3.98 g, 1.5 mL, 1.5 mmol, 3.5 eq) dropwise at 0° C. Let stir at 0° C. for 15 min and at room temperature for 3 hrs. The reaction mixture was cooled to 0° C. and methanol (˜3 mL) was added dropwise. Let stir at 0° C. for ˜15 min. Then, the mixture was added to saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound 3 (134 mg, 76% yield) as an off-white foam. LCMS: m/z 394.3 [M+H]+, ESI pos.

### Step 4: (3R or 3S)-1-[5-[2-hydroxy-6-methyl-4-(trifluoromethyl)phenyl]oxazolo[4,5-b]pyridin-2-yl]piperidin-3-ol and (3S or 3R)-1-[5-[2-hydroxy-6-methyl-4-(trifluoromethyl)phenyl]oxazolo[4,5-b]pyridin-2-yl]piperidin-3-ol

Racemate (rac)-1-[5-[2-hydroxy-6-methyl-4-(trifluoromethyl)phenyl]oxazolo[4,5-b]pyridin-2-yl]piperidin-3-ol (Example 3, step 3) (100 mg, 0.254 mmol) was separated by chiral SFC (column: OJ-H, 5 μm, 250×20 mm), eluent B: 15% methanol) to afford the two enantiomers example 3A (first eluting, Rt=3.65 min) (44 mg, 42% yield) as a white solid; m/z 394.3 [M+H]+, ESI pos and example 3B (second eluting, Rt=3.96 min) after trituration with a mixture of ethyl acetate/heptane=1:1 (23 mg, 21% yield, 90% purity) as an off-white solid. LCMS: m/z 394.3 [M+H]+, ESI pos.

### Example 4

### 3-Methyl-2-(2-morpholinooxazolo[4,5-b]pyridin-5-yl)-5-(trifluoromethyl)phenol

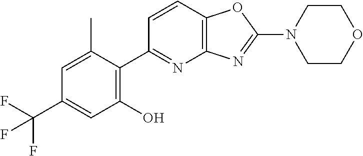 [SMILES: Cc1cc(C(F)(F)F)cc(O)c1-c1ccc2oc(N3CCOCC3)nc2n1]

### Step 1: 5-Chloro-2-morpholino-oxazolo[4,5-b]pyridine

To a mixture of 5-chloro-2-(methylthio)oxazolo[4,5-b]pyridine (CAS #1783370-92-2, 100 mg, 0.473 mmol, 1.0 eq) in 1,4-dioxane (1.0 mL) was added morpholine (CAS #110-91-8, 0.046 mL, 0.528 mmol, 1.12 eq) followed by triethylamine (0.074 mL, 0.531 mmol, 1.12 eq). The brown solution was stirred at 90° C. for 16 hrs. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 80% ethyl acetate in heptane) to afford the title compound (97 mg, 81% yield) as an off-white solid. LCMS: m/z 240.1 [M+H]⁺, ESI pos.

### Step 2: 5-[2-Methoxy-6-methyl-4-(trifluoromethyl)phenyl]-2-morpholino-oxazolo[4,5-b]pyridine

A mixture of 5-chloro-2-morpholino-oxazolo[4,5-b]pyridine (Example 4, step 1) (94 mg, 0.373 mmol, 1.0 eq), 2-[2-methoxy-6-methyl-4-(trifluoromethyl)phenyl]-4,4,5,5-tetramethyl-1,3,2-dioxaborolane (CAS #2557358-25-3, 165 mg, 0.522 mmol, 1.4 eq), potassium carbonate (260 mg, 1.88 mmol, 5.05 eq) and 1,1′-bis(diphenylphosphino)ferrocene-palladium(ii)dichloride dichloromethane complex (37 mg, 0.045 mmol, 0.122 eq) in 1,4-dioxane (2.4 mL) and water (1.2 mL) was flushed with argon and stirred at 110° C. for 3 hrs and at room temperature for 16 hrs.

The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 70% ethyl acetate in heptane) to afford the title compound (112 mg, 65% yield, 85% purity) as a light yellow foam. LCMS: m/z 394.3 [M+H]+, ESI pos.

### Step 3: 3-Methyl-2-(2-morpholinooxazolo[4,5-b]pyridin-5-yl)-5-(trifluoromethyl)phenol

To a solution of 5-[2-methoxy-6-methyl-4-(trifluoromethyl)phenyl]-2-morpholino-oxazolo[4,5-b]pyridine (Example 4, step 2) (112 mg, 0.242 mmol, 1.0 eq, 85% purity) in dichloromethane (0.460 mL) was added boron tribromide (1 M solution in dichloromethane) (2.28 g, 0.860 mL, 0.860 mmol, 3.55 eq) dropwise at 0° C. Let stir at 0° C. for 15 min and at room temperature for 3 hrs. The reaction mixture was cooled to 0° C. and methanol (˜3 mL) was added dropwise. Let stir at 0° C. for ˜15 min. Then, the mixture was added to saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 50% ethyl acetate in heptane) to afford the title compound (48 mg, 50% yield) as an off-white solid. LCMS: m/z 380.2 [M+H]+, ESI pos.

### Example 5

### (rac)-[2-[2-(Hydroxymethyl)morpholin-4-yl]oxazolo[4,5-b]pyridin-5-yl]-3-methyl-5-(trifluoromethyl)phenol

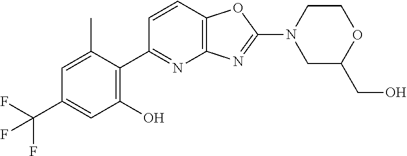 [SMILES: Cc1cc(C(F)(F)F)cc(O)c1-c1ccc2oc(N3CCOC(CO)C3)nc2n1]

### Step 1: (rac)-[4-(5-Chlorooxazolo[4,5-b]pyridin-2-yl)morpholin-2-yl]methanol

To a mixture of 5-chloro-2-(methylthio)oxazolo[4,5-b]pyridine (CAS #1783370-92-2, 80 mg, 0.379 mmol, 1.0 eq) in 1,4-dioxane (0.80 mL) was added (rac)-morpholin-2-ylmethanol (CAS #103003-01-6, 50 mg, 0.427 mmol, 1.13 eq) followed by triethylamine (0.060 mL, 0.430 mmol, 1.14 eq). The brown solution was stirred at 90° C. for 16 hrs. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% ethyl acetate in heptane) to afford the title compound (90 mg, 84% yield) as a colorless oil. LCMS: m/z 270.1 [M+H]+, ESI pos.

### Step 2: (rac)-[2-[2-(Hydroxymethyl)morpholin-4-yl]oxazolo[4,5-b]pyridin-5-yl-3-methyl-5-(trifluoromethyl)phenol

A mixture of (rac)-[4-(5-chlorooxazolo[4,5-b]pyridin-2-yl)morpholin-2-yl]methanol (Example 5, step 1) (87 mg, 0.306 mmol, 1 eq), 3-methyl-2-(4,4,5,5-tetramethyl-1,3,2-dioxaborolan-2-yl)-5-(trifluoromethyl)phenol (CAS #2557358-38-8, 134 mg, 0.444 mmol, 1.45 eq), XPhos Pd G3 (26 mg, 0.031 mmol, 0.100 eq) and cesium carbonate (300 mg, 0.921 mmol, 3.0 eq) in 1,4-dioxane (1.2 mL) and water (0.30 mL) was flushed with argon and stirred at 100° C. for 2 h and at room temperature for 16 hrs. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane). All fractions containing product were combined and concentrated in vacuo. The residue was adsorbed on ISOLUTE HM-N and repurified by flash chromatography (silica gel, 12 g, gradient 0% to 50% (dichloromethane:methanol:NH₄OH 9:1:0.05) in dichloromethane) to afford the title compound (99 mg, 75% yield) as an off-white foam. LCMS: m/z 410.3 [M+H]+, ESI pos.

### Example 6

### 3-Methyl-2-(2-piperazin-1-yloxazolo[4,5-b]pyridin-5-yl)-5-(trifluoromethyl)phenol formic acid salt

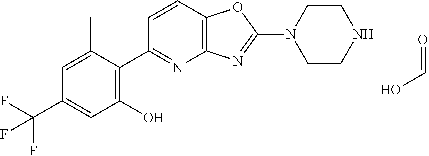 [SMILES: Cc1cc(C(F)(F)F)cc(O)c1-c1ccc2oc(N3CCNCC3)nc2n1 ; O=CO]

### Step 1: 5-[2-Methoxy-6-methyl-4-(trifluoromethyl)phenyl]-2-piperazin-1-yl-oxazolo[4,5-b]pyridine

A mixture of 5-chloro-2-piperazin-1-yl-oxazolo[4,5-b]pyridine (CAS #2504954-78-1, WO2020207941A1) (119.4 mg, 0.500 mmol, 1 eq), 2-[2-methoxy-6-methyl-4-(trifluoromethyl)phenyl]-4,4,5,5-tetramethyl-1,3,2-dioxaborolane (CAS #2557358-25-3, 268 mg, 0.848 mmol, 1.7 eq), potassium carbonate (330 mg, 2.39 mmol, 4.78 eq) and 1,1′-bis(diphenylphosphino)ferrocene-palladium(ii)dichloride dichloromethane complex (42.7 mg, 0.058 mmol, 0.116 eq) in 1,4-dioxane (3 mL) and water (1.5 mL) was flushed with argon and stirred at 110° C. for 5 hrs. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product (dark brown solid, 304.5 mg) was adsorbed on ISOLUTE HM-N and purified by flash chromatography (silica gel, 12 g, gradient 0% to 15% MeOH in dichloromethane) to afford the title compound (158.1 mg, 77% yield) as a brown solid. LCMS: m/z 393.3 [M+H]+, ESI pos.

### Step 2: 3-Methyl-2-(2-piperazin-1-yloxazolo[4,5-b]pyridin-5-yl)-5-(trifluoromethyl)phenol; formic acid salt

To a solution of aforementioned 5-[2-methoxy-6-methyl-4-(trifluoromethyl)phenyl]-2-piperazin-1-yl-oxazolo[4,5-b]pyridine (Example 6, step 1) (123.1 mg, 0.314 mmol, 1.0 eq, 95% purity) in dichloromethane (1.6 mL) was added dropwise boron tribromide (1 M solution in dichloromethane) (1.1 mL, 1.10 mmol, 3.5 eq) dropwise at −75° C. (bath with dry ice and acetone). Let stir at that temperature for 1 hr and let it warm-up to rt overnight. The reaction mixture was cooled to −75° C. and methanol (˜20 mL) was added dropwise. The red solution was stirred for ˜5 min. Then, the mixture was added to saturated aq. NaHCO₃-solution (until pH was adjusted to 7) and extracted two times with dichloromethane. The combined organic layers were dried over sodium sulfate, filtered and concentrated in vacuo. The crude product was purified by reverse phase HPLC (column: YMC-Triart C₁₈, 12 nm, 5 μm, 100×30 mm; eluent acetonitrile/water+0.1% HCOOH) to afford the title compound as a formic salt (50.6 mg, 50% yield, 93% purity) as a colorless solid. LCMS: m/z 377.3 [M+H]+, ESI pos.

### Example 7

### 3-Methyl-2-[2-[[3-(methylamino)cyclohexyl]amino]oxazolo[4,5-b]pyridin-5-yl]-5-(trifluoromethyl)phenol

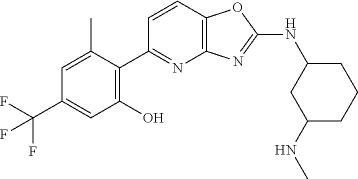 [SMILES: CNC1CCCC(Nc2nc3nc(-c4c(C)cc(C(F)(F)F)cc4O)ccc3o2)C1]

### Step 1: tert-butyl N-[3-[(5-chlorooxazolo[4,5-b]pyridin-2-yl)amino]cyclohexyl]-N-methyl-carbamate

To a mixture of 5-chloro-2-(methylthio)oxazolo[4,5-b]pyridine (CAS #1783370-92-2, 88 mg, 0.439 mmol, 1.0 eq) in 1,4-dioxane (0.90 mL) was added tert-butyl N-(3-aminocyclohexyl)-N-methyl-carbamate (CAS #1783996-31-5, 100 mg, 0.438 mmol, 0.99 eq) followed by triethylamine (67.3 μL, 0.438 mmol, 1.1 eq). The clear yellow solution was stirred at 90° C. for 24 hrs. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was purified by flash chromatography (silica gel, 12 g, gradient 0% to 10% methanol in dichloromethane) to afford the title compound (115 mg, 55% yield, 80% purity) as a light yellow oil. LCMS: m/z 381.3 [M+H]⁺, ESI pos.

### Step 2: tert-Butyl N-[3-[[5-[2-methoxy-6-methyl-4-(trifluoromethyl)phenyl]oxazolo[4,5-b]pyridin-2-yl]amino]cyclohexyl]-N-methyl-carbamate

A mixture of aforementioned tert-butyl N-[3-[(5-chlorooxazolo[4,5-b]pyridin-2-yl)amino]cyclohexyl]-N-methyl-carbamate (Example 7, Step 1) (115 mg, 0.242 mmol, 1 eq), 2-[2-methoxy-6-methyl-4-(trifluoromethyl)phenyl]-4,4,5,5-tetramethyl-1,3,2-dioxaborolane (CAS #2557358-25-3, 129.8 mg, 0.411 mmol, 1.7 eq), potassium carbonate (159.5 mg, 1.15 mmol, 4.78 eq) and 1,1′-bis(diphenylphosphino)ferrocene-palladium(ii)dichloride dichloromethane complex (22.8 mg, 0.028 mmol, 0.116 eq) in 1,4-dioxane (1.4 mL) and water (0.7 mL) was flushed with argon and stirred at 110° C. for 24 hrs (for reaction completion 0.5 eq for the catalyst and the base were added; also the reaction mixture was stored in the fridge for 3 d before reaction completion). The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product (dark brown oil) was adsorbed on ISOLUTE HM-N and purified by flash chromatography (silica gel, 12 g, gradient 0% to 15% MeOH in dichloromethane) to afford the title compound (78.9 mg, 52% yield, 85% purity) as green solid. LCMS: m/z 535.5 [M+H]+, ESI pos.

### Step 3: 3-Methyl-2-[2-[[3-(methylamino)cyclohexyl]amino]oxazolo[4,5-b]pyridin-5-yl]-5-(trifluoromethyl)phenol

To a solution of aforementioned tert-butyl N-[3-[[5-[2-methoxy-6-methyl-4-(trifluoromethyl)phenyl]oxazolo[4,5-b]pyridin-2-yl]amino]cyclohexyl]-N-methyl-carbamate (Example 7, step 2) (78.9 mg, 0.148 mmol, 1.0 eq) in dichloromethane (2 mL) was added dropwise boron tribromide (1 M solution in dichloromethane) (664.2 μL, 0.664 mmol, 4.5 eq) dropwise at 0° C. (bath with dry ice and acetone). Let stir at that temperature for 1 hr and let it warm-up to rt over a day. The reaction mixture was cooled to 0° C. and few drops of methanol were added. The clear yellow solution was stirred for ˜5 min. Then, the mixture was added to saturated aq. NaHCO₃-solution (until pH was basic) and extracted three times with dichloromethane. The combined organic layers were dried over sodium sulfate, filtered and concentrated in vacuo. The crude product was purified by reverse phase HPLC (column: YMC-Triart C₁₈, 12 nm, 5 μm, 100×30 mm; eluent acetonitrile/water+0.1% triethylamine) to afford the title compound most likely as a diastereomeric mixture cis/trans=2:1 (30 mg, 30% yield, 85% purity) as an off-white solid. LCMS: m/z 421.1853 [M+H]⁺, ESI pos.

### Example 8

### 3-Methyl-2-(2-piperazin-1-yl-1H-imidazo[4,5-b]pyridin-5-yl)-5-(trifluoromethyl)phenol;2,2,2-trifluoroacetic acid

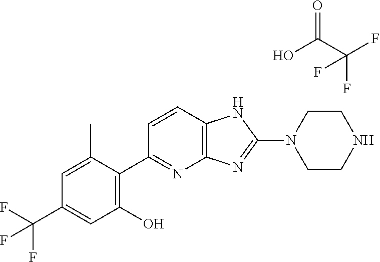

### Step 1: 5-Bromo-TH-imidazo[4,5-b]pyridin-2-ol

To a mixture of 6-bromopyridine-2,3-diamine (1.0 g, 5.32 mmol, 1 eq) in 1,4-dioxane (30 mL) was added carbonyl diimidazole (974.36 mg, 6.91 mmol, 1.3 eq) under N₂ at 25° C. Then the mixture was heated at 90° C. and stirred for 4 hrs. The mixture was concentrated in vacuum. The crude product was triturated with MTBE (10 mL*3). The solid was collected to afford the title compound (770 mg, 68% yield) as a black solid. ¹H NMR (400 MHz, DMSO-d₆) δ 11.57 (br. s, 1H), 11.01 (s, 1H), 7.18 (d, 1H), 7.12 (d, 1H).

### Step 2: 5-Bromo-2-chloro-1H-imidazo[4,5-b]pyridine

A mixture of aforementioned 5-bromo-1H-imidazo[4,5-b]pyridin-2-ol (Example 8, step 1) (300.0 mg, 1.4 mmol, 1 eq) and POCl₃ (4298.3 mg, 28.03 mmol, 20 eq) in toluene (2 mL) was stirred at 110° C. for 5 hrs. The mixture was concentrated under reduced pressure to afford the title compound (400 mg, 18% yield, HCl salt) as a brown solid. LCMS: m/z 233.8 [M+H]+, ESI pos.

### Step 3: tert-butyl 4-(5-bromo-1H-imidazo[4,5-b]pyridin-2-yl)piperazine-1-carboxylate

To a mixture of aforementioned 5-bromo-2-chloro-1H-imidazo[4,5-b]pyridine;hydrochloride (Example 8, step 2) (400.0 mg, 0.250 mmol, 1.0 eq), tert-butyl piperazine-1-carboxylate (231.59 mg, 1.24 mmol, 5 eq) in N-methylpyrrolidone (10 mL) was added K₂CO₃ (274.6 mg, 1.99 mmol, 8.0 eq) under N2 at 25° C. Then the mixture was stirred at 90° C. for 4 hrs. The mixture was filtered and the filtrate was concentrated in vacuum. The crude product was purified by reversed-phase flash (0.1% TFA condition) to afford the title compound (40 mg, 38% yield) as a brown solid. LCMS: m/z 384.0 [M+2H]+, ESI pos.

### Step 4: tert-butyl 4-(5-(2-hydroxy-6-methyl-4-(trifluoromethyl)phenyl)-1H-imidazo[4,5-b]pyridin-2-yl)piperazine-1-carboxylate

To a mixture of aforementioned tert-butyl 4-(5-bromo-1H-imidazo[4,5-b]pyridin-2-yl)piperazine-1-carboxylate (Example 8, step 3) (40.0 mg, 0.100 mmol, 1.0 eq), (2-hydroxy-6-methyl-4-(trifluoromethyl) phenyl)boronic acid (34.5 mg, 0.160 mmol, 1.5 eq), Na₂CO₃ (33.3 mg, 0.310 mmol, 3 eq) in 1,4-dioxane (2 mL)/water (1 mL) was added Pd(dppf)Cl₂ (7.66 mg, 0.010 mmol, 0.100 eq) under N₂ at 25° C. Then the mixture was stirred for 2 hrs at 95° C. The mixture was poured into water (10 mL) and extracted with ethyl acetate (50 mL*3). The combined organic layers were washed with brine (20 mL), dried over anhydrous sodium sulfate, filtered, and the filtrate was concentrated under reduced pressure. The residue was purified by column silica gel using petroleum ether/ethyl acetate=1/1 to afford the title compound (20 mg, 33% yield) as a light yellow solid. LCMS: m/z 478.0 [M+H]+, ESI pos.

### Step 5: 3-Methyl-2-(2-piperazin-1-yl-1H-imidazo[4,5-b]pyridin-5-yl)-5 (trifluoromethyl)phenol;2,2,2-trifluoroacetic acid

A mixture of aforementioned tert-butyl 4-(5-(2-hydroxy-6-methyl-4-(trifluoromethyl)phenyl)-1H-imidazo[4,5-b]pyridin-2-yl)piperazine-1-carboxylate (Example 8, step 4) (20.0 mg, 0.030 mmol, 1 eq), TFA (94.2 mg, 1.02 mmol, 30 eq) in Dichloromethane (1 mL) was stirred for 2 hrs at 25° C. The reaction mixture was concentrated under reduced pressure. The residue was purified by prep-HPLC (Column: 3 Phenomenex Luna C18 75*30 mm*3 um, water (TFA)-acetonitrile, 12%˜42%, 7 min) to afford the title compound (4.69 mg, 27% yield) as a light yellow solid. m/z 378.0 [M+H]+, ESI pos.

### Example 9

### (rac)-5-(5-(2-Hydroxy-6-methyl-4-(trifluoromethyl)phenyl)oxazolo[4,5-b]pyridin-2-yl) piperidin-2-one

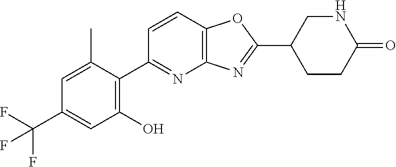 [SMILES: Cc1cc(C(F)(F)F)cc(O)c1-c1ccc2oc(C3CCC(=O)NC3)nc2n1]

### Step 1: (rac)-5-(5-Bromooxazolo[4,5-b]pyridin-2-yl)piperidin-2-one

The solution of 2-amino-6-bromopyridin-3-ol (CAS #934758-27-7, 150.0 mg, 0.790 mmol, 1.0 eq) and (rac)-6-oxopiperidine-3-carboxylic acid (CAS #22540-50-7, 113.6 mg, 0.790 mmol, 1.0 eq) in polyphosphoric acid (1.0 mL) was stirred at 135° C. for 12 hrs. The above reaction mixture was diluted with cold water (50 mL) and treated with saturated sodium carbonate to adjust pH to about 10, then extracted with ethyl acetate (20 mL*3). The combined organic phase was washed with brine (50 mL), dried over anhydrous sodium sulfate, filtered and concentrated under reduced pressure. The crude product was purified by reversed phase flash (0.1% TFA condition) to afford the title compound (50 mg, 11% yield) as a yellow solid. LCMS: m/z 296.0 [M+2H]+, ESI pos.

### Step 2: (rac)-5-(5-(2-Hydroxy-6-methyl-4-(trifluoromethyl)phenyl)oxazolo[4,5-b]pyridin-2-yl) piperidin-2-one

To a solution of (rac)-5-(5-bromooxazolo[4,5-b]pyridin-2-yl)piperidin-2-one (Example 9, step 1) (40.0 mg, 0.140 mmol, 1.0 eq) in 1,4-dioxane (2 mL) and water (0.40 mL) was added (2-hydroxy-6-methyl-4-(trifluoromethyl)phenyl)boronic acid (59.4 mg, 0.270 mmol, 2.0 eq), Na₂CO₃ (42.9 mg, 0.410 mmol, 3.0 eq) and Pd(dppf)Cl₂ (19.8 mg, 0.030 mmol, 0.20 eq). The above reaction mixture was stirred at 100° C. for 2 hrs under N₂ atmosphere. The above reaction mixture was filtered and the filtrate was concentrated under reduced pressure. The crude product was purified by prep-HPLC (Column: Waters Xbridge 150*25 mm*5 μm; conditions water (ammonia hydroxide v/v)-acetonitrile; Begin B 18 End B 48; Gradient Time (min): 9; 100% B Hold time (min): 2; FlowRate (m/min): 25) to afford the title compound (2.04 mg, 4% yield) as a white solid. LCMS: m/z 392.0 [M+H]⁺ ESI pos.

### Example 10

### (rac)-3-Methyl-2-[2-[3-(methylamino)-1-piperidyl]oxazolo[4,5-b]pyridin-5-yl]-5-(trifluoromethyl) phenol

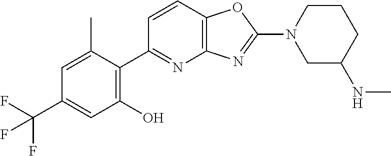 [SMILES: CNC1CCCN(c2nc3nc(-c4c(C)cc(C(F)(F)F)cc4O)ccc3o2)C1]

### Step 1: (rac)-tert-Butyl N-[1-(5-chlorooxazolo[4,5-b]pyridin-2-yl)-3-piperidyl]-N-methyl-carbamate

To a mixture of 5-chloro-2-(methylthio)oxazolo[4,5-b]pyridine (CAS #1783370-92-2, 90 mg, 0.449 mmol, 1 eq) in 1,4-dioxane (0.90 mL) was added (rac)-N-methyl-N-(3-piperidyl)carbamic acid tert-butyl ester (CAS #172478-01-2, 95 μL, 0.449 mmol, 1 eq) followed by triethylamine (69 μL, 0.493 mmol, 1.1 eq). The clear brown solution was stirred at 90° C. for two days. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was purified by flash chromatography (silica gel, 12 g, gradient 0% to 5% methanol in dichloromethane) to afford the title compound (139.2 mg, 81% yield) as an off-white solid. LCMS: m/z 367.3 [M+H]⁺, ESI pos.

### Step 2: (rac)-tert-Butyl N-[1-[5-[2-methoxy-6-methyl-4-(trifluoromethyl)phenyl]oxazolo[4,5-b]pyridin-2-yl]-3-piperidyl]-N-methyl-carbamate

To a mixture of aforementioned (rac)-tert-Butyl N-[1-(5-chlorooxazolo[4,5-b]pyridin-2-yl)-3-piperidyl]-N-methyl-carbamate (Example 10, step 10) (139.2 mg, 0.379 mmol, 1.0 eq), 2-[2-methoxy-6-methyl-4-(trifluoromethyl)phenyl]-4,4,5,5-tetramethyl-1,3,2-dioxaborolan (179.9 mg, 0.569 mmol, 1.5 eq), Na₂CO₃ (250.7 mg, 1.81 mmol, 4.78 eq) in 1,4-dioxane (2.3 mL)/water (1.1 mL) was added Pd(dppf)Cl₂ (35.9 mg, 0.044 mmol, 0.116 eq) under N2 at 25° C. Then the mixture was stirred for 2 days at 110° C. The mixture was poured into water and extracted with ethyl acetate. The combined organic layers were washed with brine, dried over anhydrous sodium sulfate, filtered, and the filtrate was concentrated under reduced pressure. The crude product was purified by flash chromatography (silica gel, 12 g, gradient 0% to 10% methanol in dichloromethane) to afford the title compound (122.8 mg, 48% yield, 77% purity) as a dark brown solid. LCMS: m/z 521.5 [M+H]⁺ ESI pos.

### Step 3: (rac)-3-Methyl-2-[2-[3-(methylamino)-1-piperidyl]oxazolo[4,5-b]pyridin-5-yl]-5-(trifluoromethyl)phenol

A mixture of aforementioned (rac)-tert-butyl N-[1-[5-[2-methoxy-6-methyl-4-(trifluoromethyl)phenyl]oxazolo[4,5-b]pyridin-2-yl]-3-piperidyl]-N-methyl-carbamate (Example 10, step 2) (78.9 mg, 0.152 mmol, 1.0 eq), 3-methyl-2-(4,4,5,5-tetramethyl-1,3,2-dioxaborolan-2-yl)-5-(trifluoromethyl)phenol (CAS #2557358-38-8, 682 μL, 0.682 mmol, 4.5 eq), XPhos Pd G3 (11.3 mg, 0.013 mmol, 0.098 eq) and cesium carbonate (12.2 mg, 0.373 mmol, 2.88 eq) in 1,4-dioxane (0.5 mL) and water (0.2 mL) was flushed with argon and stirred at 100° C. for 5 hrs.

The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product (yellow oil, 114 mg) was purified by HPLC (Column: Phenomenex Gemini NX C¹⁸-5 μm-110 Å-100×30 mm; mobile phase H₂O+0.1% triethylamine/acetonitrile (gradient over 15 min)) to afford the title compound (32.4 mg, 53% yield, 90% purity) as an off-white solid. LCMS: m/z 419.5 [M−H]⁻, ESI neg.

### Example 11

### (rac)-3-Methyl-2-[2-(tetrahydropyran-3-ylamino)oxazolo[4,5-b]pyridin-5-yl]-5-(trifluoromethyl)phenol

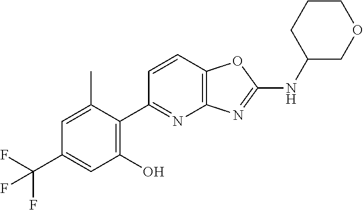 [SMILES: Cc1cc(C(F)(F)F)cc(O)c1-c1ccc2oc(NC3CCCOC3)nc2n1]

### Step 1: (rac)-5-Chloro-N-tetrahydropyran-3-yl-oxazolo[4,5-b]pyridin-2-amine

To a mixture of 5-chloro-2-(methylthio)oxazolo[4,5-b]pyridine (CAS #1783370-92-2, 100 mg, 0.498 mmol, 1 eq) in 1,4-dioxane (2.8 mL) was added (rac)-tetrahydropyran-3-ylamine (55.5 mg, 57.6 μL, 0.548 mmol, 1.1 eq) followed by triethylamine (56.5 mg, 77.8 μL, 0.558 mmol, 1.12 eq). The brown solution was stirred at 90° C. overnight. The reaction mixture was cooled to room temperature and extracted with ˜40 mL ethyl acetate, and ˜5 mL saturated NaHCO₃-solution. The aqueous layer was backextracted with ˜40 mL ethyl acetate. The organic layers were washed with ˜5 mL water and ˜5 mL brine. The combined organic layers were dried over sodium sulfate, filtered and concentrated in vacuo. The crude product was purified by flash chromatography (silica gel, 4 g, gradient 0% to 10% methanol in dichloromethane) to afford the title compound (63 mg, 59%) as white solid. LCMS: m/z=254.1 [M+H]⁺, ESI pos.

### Step 2: (rac)-3-Methyl-2-[2-(tetrahydropyran-3-ylamino)oxazolo[4,5-b]pyridin-5-yl]-5-(trifluoromethyl)phenol

To a solution of aforementioned (rac)-(5-chlorooxazolo[4,5-b]pyridin-2-yl)-tetrahydropyran-3-yl-amine (Example 11, step 1) (50 mg, 0.197 mmol, 1.0 eq) and 3-methyl-2-(4,4,5,5-tetramethyl-1,3,2-dioxaborolan-2-yl)-5-(trifluoromethyl)phenol (CAS #2557358-38-8, 86.5 mg, 71.5 μL, 0.286 mmol, 1.45 eq) in 1,4-dioxane, extra dry (0.8 mL) and water (0.2 mL) was added under argon cesium carbonate (184.7 mg, 0.567 mmol, 2.88 eq) followed by XPhos Pd G3 (17.2 mg, 0.019 mmol, 0.098 eq). The reaction mixture was flushed with argon and stirred at 100° C. for one hr. After reaction completion, the mixture was cooled to room temperature and extracted with ˜ 5 mL ethyl acetate and ˜5 mL water. The aqueous layer was backextracted with ˜5 mL ethyl acetate. The organic layers were washed with ˜5 mL water and ˜ 5 mL brine. The combined organic layers were dried over sodium sulfate, filtered and concentrated in vacuo. The crude product was adsorbed on ISOLUTE HM-N and purified by flash chromatography (silica gel, 4 g, gradient 0% to 10% methanol in dichloromethane). All fractions containing product were combined to afford the title compound (44 mg, 57%) as white solid. LCMS: m/z 394.3 [M+H]⁺, ESI pos.

### Example 12

### 1-[5-[2-Hydroxy-6-methyl-4-(trifluoromethyl)phenyl]oxazolo[4,5-b]pyridin-2-yl]azetidin-3-ol

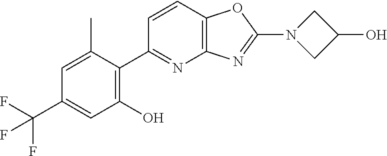 [SMILES: Cc1cc(C(F)(F)F)cc(O)c1-c1ccc2oc(N3CC(O)C3)nc2n1]

### Step 1: 1-(5-Chlorooxazolo[4,5-b]pyridin-2-yl)azetidin-3-ol

To a mixture of 5-chloro-2-(methylthio)oxazolo[4,5-b]pyridine (CAS #1783370-92-2, 0.100 g, 0.473 mmol, 1.0 eq) in 1,4-dioxane (1 mL) was added azetidin-3-ol (CAS #45347-82-838.3 mg, 0.524 mmol, 1.11 eq) followed by triethylamine (53.7 mg, 74 μL, 0.531 mmol, 1.12 eq). The brown solution was stirred at 90° C. overnight. The reaction mixture was cooled to room temperature and extracted with ˜20 mL ethyl acetate, 20 mL dichloromethane (solubility problems) and ˜5 mL saturated NaHCO₃-solution. The aqueous layer was backextracted with ˜40 mL dichloromethane. The organic layers were washed with ˜5 mL water and ˜5 mL brine. The combined organic layers were dried over sodium sulfate, filtered and concentrated in vacuo. The crude product was purified by flash chromatography (silica gel, 4 g, gradient 0% to 10% methanol in dichloromethane) to afford the title compound (63 mg, 59%) as a white solid. LCMS: m/z=226.1 [M+H]⁺, ESI pos.

### Step 2: 1-[5-[2-Hydroxy-6-methyl-4-(trifluoromethyl)phenyl]oxazolo[4,5-b]pyridin-2-yl]azetidin-3-ol

To a solution of 1-(5-chlorooxazolo[4,5-b]pyridin-2-yl)azetidin-3-ol (50 mg, 0.222 mmol, 1.0 eq) and 3-methyl-2-(4,4,5,5-tetramethyl-1,3,2-dioxaborolan-2-yl)-5-(trifluoromethyl)phenol (97.3 mg, 80.4 μL, 0.322 mmol, 1.45 eq) in 1,4-dioxane, extra dry (0.9 mL) and water (0.2 mL) was added under argon cesium carbonate (207.7 mg, 0.637 mmol, 2.88 eq) followed by XPhos Pd G3 (19.4 mg, 0.022 mmol, 0.098 eq). The reaction mixture was flushed with argon and stirred at 100° C. for one hr. After reaction completion, the mixture was cooled to room temperature and extracted with ˜5 mL ethyl acetate and ˜5 mL water. The aqueous layer was backextracted with ˜5 mL ethyl acetate. The organic layers were washed with ˜5 mL water and ˜5 mL brine. The combined organic layers were dried over sodium sulfate, filtered and concentrated in vacuo. The crude product was adsorbed on ISOLUTE HM-N and purified by flash chromatography (silica gel, 4 g, gradient 0% to 10% methanol in dichloromethane). All fractions containing product were combined. The reaction mixture was given to HPLC prep. Affording after lyophilisation the title compound (10.3 mg, 12%) as white solid. LCMS: m/z=366.2 [M+H]⁺, ESI pos.

### Example 13

### 2-[2-[(3-Hydroxycyclohexyl)amino]oxazolo[4,5-b]pyridin-5-yl]-3-methyl-5-(trifluoromethyl)phenol

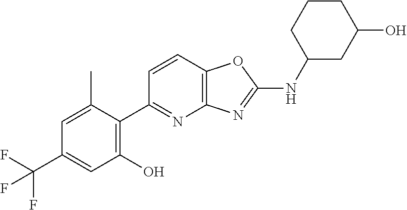 [SMILES: Cc1cc(C(F)(F)F)cc(O)c1-c1ccc2oc(NC3CCCC(O)C3)nc2n1]

### Step 1: 3-[(5-Chlorooxazolo[4,5-b]pyridin-2-yl)amino]cyclohexanol

To a solution of 5-chloro-2-(methylthio)oxazolo[4,5-b]pyridine (0.080 g, 0.379 mmol, 1 eq) in 1,4-dioxane (0.8 mL) was added 3-aminocyclohexanol (CAS #6850-39-1, 48.2 mg, 0.419 mmol, 1.11 eq) followed by triethylamine (42.98 mg, 59.2 uL, 0.425 mmol, 1.12 eq). The brown solution was stirred at 90° C. for two days. LC-MS showed 43% starting material and 51% product. 3-aminocyclohexanol (23.9 mg, 0.208 mmol, 0.550 eq) was added to the reaction mixture and stirred for five hrs. LC-MS showed a small educt peak. 3-aminocyclohexanol (23.9 mg, 0.208 mmol, 0.550 eq) was added to the reaction mixture and it was stirred at 90° C. overnight. After reaction completion, the mixture was cooled to room temperature and extracted with ˜40 mL ethyl acetate and ˜5 mL saturated NaHCO₃-solution. The aqueous layer was back extracted with ˜40 mL ethyl acetate. The organic layers were washed with ˜5 mL water and ˜5 mL brine. The combined organic layers were dried over sodium sulfate, filtered and concentrated in vacuo. The crude product was purified by flash chromatography (silica gel, 4 g, gradient 0% to 10% methanol in dichloromethane) to afford the title compound (79.6 mg, 79%) as light yellow solid. LCMS: m/z=268.1 [M+H]⁺, ESI pos.

### Step 2: 2-[2-[(3-Hydroxycyclohexyl)amino]oxazolo[4,5-b]pyridin-5-yl]-3-methyl-5-(trifluoromethyl) phenol

To a solution of aforementioned 3-[(5-chlorooxazolo[4,5-b]pyridin-2-yl)amino]cyclohexanol (Example 13, step 1) (75 mg, 0.280 mmol, 1.0 eq) and 3-methyl-2-(4,4,5,5-tetramethyl-1,3,2-dioxaborolan-2-yl)-5-(trifluoromethyl)phenol (122.9 mg, 101.6 μL, 0.407 mmol, 1.45 eq) in 1,4-dioxane (1 mL) and water (0.3 mL) was added under argon cesium carbonate (262.5 mg, 0.806 mmol, 2.88 eq) followed by XPhos Pd G3 (24.5 mg, 0.027 mmol, 0.098 eq). The reaction mixture was flushed with argon and stirred at 100° C. for one hr. The reaction mixture was cooled to room temperature and extracted with ˜5 mL ethyl acetate and ˜5 mL water. The aqueous layer was backextracted with ˜5 mL ethyl acetate. The organic layers were washed with ˜5 mL water and ˜5 mL brine. The combined organic layers were dried over sodium sulfate, filtered and concentrated in vacuo. The crude product was adsorbed on ISOLUTE HM-N and purified by flash chromatography (silica gel, 4 g, gradient 0% to 70% ethyl acetate in heptane) affording the title compound (80 mg, 67%) as yellow solid. LCMS: m/z=408.3 [M+H]⁺, ESI pos.

### Example 14

### 2-[2-[[(3R)-1-Ethyl-3-piperidyl]amino]oxazolo[4,5-b]pyridin-5-yl]-3-methyl-5-(trifluoromethyl)phenol

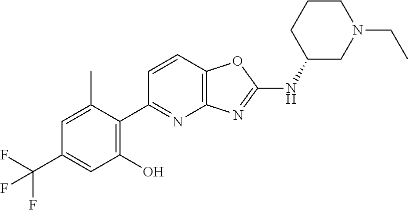 [SMILES: CCN1CCC[C@@H](Nc2nc3nc(-c4c(C)cc(C(F)(F)F)cc4O)ccc3o2)C1]

### Step 1: 5-Chloro-N-[(3R)-1-ethyl-3-piperidyl]oxazolo[4,5-b]pyridin-2-amine

To a mixture of 5-chloro-2-(methylthio)oxazolo[4,5-b]pyridine (CAS #1783370-92-2, 320 mg, 1.52 mmol, 1 eq) in 1,4-dioxane (3.2 mL) was added (3R)-1-ethylpiperidin-3-amine (CAS #1020396-26-2, 240 mg, 1.76 mmol, 1.16 eq) followed by triethylamine (0.240 mL, 1.72 mmol, 1.14 eq). The brown solution was stirred at 90° C. for 16 hrs. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in dichloromethane) to afford the title compound (405 mg, 90% yield) as an off-white solid. LCMS: m/z 281.2 [M+H]⁺, ESI pos.

### Step 2: 2-[2-[[(3R)-1-Ethyl-3-piperidyl]amino]oxazolo[4,5-b]pyridin-5-yl]-3-methyl-5-(trifluoromethyl)phenol

A mixture of 5-chloro-N-[(3R)-1-ethyl-3-piperidyl]oxazolo[4,5-b]pyridin-2-amine (Example 14, step 1) (300 mg, 1.02 mmol, 1 eq), 3-methyl-2-(4,4,5,5-tetramethyl-1,3,2-dioxaborolan-2-yl)-5-(trifluoromethyl)phenol (CAS #2557358-38-8, 430 mg, 1.42 mmol, 1.4 eq), cesium carbonate (995 mg, 3.05 mmol, 3.01 eq) and XPhos Pd G3 (89 mg, 0.105 mmol, 0.10 eq) in 1,4-dioxane (4.0 mL) and water (1.0 mL) was flushed with argon and stirred at 90° C. for 16 hrs. The reaction mixture was cooled to room temperature and extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 25 g, gradient 0% to 100% (dichloromethane:methanol:NH₄OH 9:1:0.05) in dichloromethane). All fractions containing product were combined and concentrated in vacuo. The residue was triturated with ethyl acetate/heptane to afford the title compound (328 mg, 73% yield) as an off-white solid. LCMS: m/z 421.3 [M+H]⁺, ESI pos.

### Example 15

### 3-Methyl-2-[2-[[(3R)-1-methyl-3-piperidyl]amino]oxazolo[4,5-b]pyridin-5-yl]-5-(trifluoromethyl)phenol

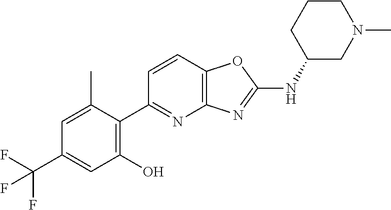 [SMILES: Cc1cc(C(F)(F)F)cc(O)c1-c1ccc2oc(N[C@@H]3CCCN(C)C3)nc2n1]

### Step 1: 5-Chloro-N-[(3R)-1-methyl-3-piperidyl]oxazolo[4,5-b]pyridin-2-amine

To a mixture of 5-chloro-2-(methylthio)oxazolo[4,5-b]pyridine (CAS #1783370-92-2, 80 mg, 0.379 mmol, 1 eq) in 1,4-dioxane (0.800 mL) was added (3R)-1-methylpiperidin-3-amine (CAS #1001353-92-9, 51 mg, 0.447 mmol, 1.18 eq) followed by triethylamine (0.061 mL, 0.438 mmol, 1.16 eq). The brown solution was stirred at 90° C. for 16 hrs. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (101 mg, 95% yield) as an off-white solid. LCMS: m/z 267.1 [M+H]⁺, ESI pos.

### Step 2: 3-Methyl-2-[2-[[(3R)-1-methyl-3-piperidyl]amino]oxazolo[4,5-b]pyridin-5-yl]-5-(trifluoromethyl)phenol

A mixture of 5-chloro-N-[(3R)-1-methyl-3-piperidyl]oxazolo[4,5-b]pyridin-2-amine (Example 15, step 1) (98 mg, 0.349 mmol, 1.0 eq), 3-methyl-2-(4,4,5,5-tetramethyl-1,3,2-dioxaborolan-2-yl)-5-(trifluoromethyl)phenol (CAS #2557358-38-8, 140 mg, 0.463 mmol, 1.33 eq), cesium carbonate (325 mg, 0.997 mmol, 2.86 eq) and XPhos Pd G3 (29 mg, 0.034 mmol, 0.098 eq) in 1,4-dioxane (1.2 mL) and water (0.3 mL) was flushed with argon and stirred at 100° C. for 2 hrs. The reaction mixture was cooled to room temperature and extracted with ethyl acetate and water. The aqueous layer was backextracted with ethyl acetate. The organic layers were washed with water and brine. The combined aqueous layers were backextracted with a mixture of dichloromethane/methanol (9:1). The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined and concentrated in vacuo. The residue was triturated with ethyl acetate/heptane to afford the title compound (88 mg, 59%) as an off-white powder. LCMS: m/z 407.3 [M+H]⁺, ESI pos.

### Example 16

### (rac)-3-Methyl-2-(2-(1-methylpiperidin-2-yl)-3H-imidazo[4,5-b]pyridin-5-yl)-5-(trifluoromethyl)phenol

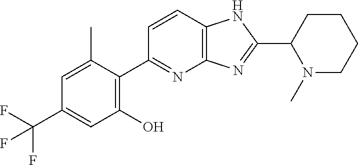 [SMILES: Cc1cc(C(F)(F)F)cc(O)c1-c1ccc2[nH]c(C3CCCCN3C)nc2n1]

### Step 1: (rac)-N-(2-amino-6-bromopyridin-3-yl)-1-methylpiperidine-2-carboxamide

To a solution of (rac)-1-methylpiperidine-2-carboxylic acid (456.88 mg, 3.19 mmol, 1 eq), 6-bromopyridine-2, 3-diamine (0.60 g, 3.2 mmol, 1.0 eq) and DIPEA (1.99 mL, 11.2 mmol, 3.5 eq) in DMF (10 mL) was added T₃P (3.05 g, 4.79 mmol, 1.5 eq, 50% purity in ethyl acetate) drop wise at 0° C. The reaction mixture was stirred under nitrogen atmosphere at 25° C. for 2 hrs. LCMS indicated the starting material still remained, so another batch of DIPEA (1.99 mL, 11.2 mmol, 3.5 eq) and T₃P (3045.92 mg, 4.79 mmol, 1.5 eq, 50% purity in ethyl acetate) was added to the mixture at 25° C. and stirred under nitrogen atmosphere at 50° C. for another 2 hrs. The reaction mixture was quenched with water (30 mL), extracted with ethyl acetate (100 mL*3). The combined organic phase was washed with brine (20 mL), dried over anhydrous sodium sulfate, filtrated and the filtrate was concentrated under reduced pressure. The residue was purified by reversed-phase flash (CombiFlash 0.1% NH₄OH aqueous-acetronitrile condition) and following lyophilization to afford the title compound (400 mg, 35% yield) as a yellow solid. LCMS: m/z 313.0 [M+H]⁺, ESI pos.

### Step 2: (rac)-5-Bromo-2-(1-methylpiperidin-2-yl)-3H-imidazo[4,5-b]pyridine

To a solution of aforementioned (rac)-N-(2-amino-6-bromopyridin-3-yl)-1-methylpiperidine-2-carboxamide (Example 16, step 1) (220 mg, 0.700 mmol, 1.0 eq) in ethanol (3 mL) and water (0.5 mL) was added NaOH (290.0 mg, 7.25 mmol, 10.3 eq) at 25° C. The reaction mixture was stirred at 100° C. for 20 hrs under nitrogen atmosphere. LCMS indicated most of the starting material still remained, and another batch of NaOH (100 mg) was added. The reaction mixture was stirred at 100° C. for another 18 hrs. The reaction mixture was diluted with 10 mL of water and extracted with ethyl acetate (100 mL*3). The combined organic phase was dried over anhydrous sodium sulfate, filtrated and the filtrate was concentrated under reduced pressure. The residue was purified by prep-HPLC (Method: Column Phenomenex luna C₁₈ 150*40 mm*15 μm; Condition: water (0.1% TFA)-acetonitrile: Begin B 23 End B 53; Gradient Time (min): 11; 100% B Hold Time (min): 2; FlowRate (mL/min): 60) and following up lyophilization to afford the title compound (100 mg, 48% yield) as a yellow solid. ¹H NMR (400 MHz, CHLOROFORM-d) δ=7.88 (d, 1H), 7.47 (d, 1H), 4.71 (d, 1H), 3.76 (d, 1H), 3.19-3.12 (m, 1H), 2.69 (s, 3H), 2.54-2.44 (m, 1H), 2.31-2.27 (m, 1H), 2.21-2.01 (m, 3H), 1.79-1.721 (m, 1H).

### Step 3: (rac)-5-(2-Methoxy-6-methyl-4-(trifluoromethyl)phenyl)-2-(1-methylpiperidin-2-yl)-3H-imidazo[4,5-b]pyridine

A mixture of aforementioned (rac)-2-(2-methoxy-6-methyl-4-(trifluoromethyl)phenyl)-4,4,5,5-tetramethyl-1, 3, 2-dioxaborolane (Example 16, step 2) (85.68 mg, 0.270 mmol, 1 eq), 5-bromo-2-(1-methylpiperidin-2-yl)-3H-imidazo[4,5-b]pyridine (80.0 mg, 0.270 mmol, 1 eq) and K₂CO₃ (201.08 mg, 1.9 mmol, 7 eq) in 1, 4-dioxane (4 mL) and water (0.800 mL) was degassed and purged with N₂ three times, and then Pd(dppf)Cl₂ (66.4 mg, 0.080 mmol, 0.300 eq) was added to the mixture. The mixture was stirred at 100° C. for 3 hrs. The reaction mixture was cooled to room temperature and concentrated under reduced pressure. The residue was purified by reversed-phase flash (CombiFlash 0.1% TFA aqueous/acetonitrile condition) and prep-HPLC (Column: 3_Phenomenex Luna C18 75*30 mm*3 um; condition: water (0.1% TFA)-acetonitrile; Begin B: 36, End B: 56; Gradient; Time (min): 8; 100% B; Hold Time (min): 2; Flow Rate (mL/min): 25) and following up lyophilization to afford the title compound (30 mg, 27% yield) as a black solid. LCMS: m/z 405.1 [M+H]⁺, ESI pos.

### Step 4: (rac)-3-Methyl-2-(2-(1-methylpiperidin-2-yl)-3H-imidazo[4,5-b]pyridin-5-yl)-5-(trifluoromethyl)phenol

To a solution of aforementioned (rac)-5-(2-methoxy-6-methyl-4-(trifluoromethyl)phenyl)-2-(1-methylpiperidin-2-yl)-3H-imidazo[4,5-b]pyridine (Example 16, step 3) (20.0 mg, 0.050 mmol, 1.0 eq) in DCM (12 mL) was added BBr₃ (2.0 mL, 20.76 mmol, 419.7 eq) drop wise at −60° C. The reaction mixture was stirred at −60° C. for 2.5 hrs and stirred at 25° C. for another 0.5 hr under nitrogen atmosphere. LCMS indicated the starting material still remained, so another batch of BBr₃ (2.0 mL, 20.76 mmol, 419.7 eq) was added to the mixture. The reaction mixture was stirred at 25° C. for additional 1 hr under nitrogen atmosphere. The reaction mixture was quenched with cooled water (5 mL) and adjusted to pH˜8 with ammonia aqueous, and then concentrated under reduced pressure. The residue was triturated with methanol (10 mL) twice, the filtrate was purified by (CombiFlash 0.1% TFA-aqueous/acetonitrile condition) and prep-HPLC (Colum: 3_Phenomenex Luna C18 75*30 mm*3 μm; Condition: water (0.1% TFA aqueous)-acetonitrile; Begin; B 21, End: B 51; Gradient Time (min): 7, 100% B; Hold Time (min): 2; Flow Rate (mL/min): 25) and following up lyophilization to afford the title compound (5.89 mg, 30% yield) as a white solid. LCMS: m/z 391.1 [M+H]⁺, ESI pos.

### Example 17

### (rac)-3-Methyl-2-(2-(pyrrolidin-2-yl)-1H-imidazo[4,5-b]pyridin-5-yl)-5-(trifluoro-methyl)phenol

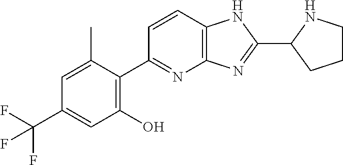 [SMILES: Cc1cc(C(F)(F)F)cc(O)c1-c1ccc2[nH]c(C3CCCN3)nc2n1]

### Step 1: (rac)-tert-Butyl 2-((2-amino-6-bromopyridin-3-yl)carbamoyl)pyrrolidine-1-carboxylate

To a solution of (rac)-(tert-butoxycarbonyl)proline (1.0 g, 4.65 mmol, 1 eq) in DMF (10 mL) was added DIPEA (1.22 g, 9.29 mmol, 2 eq) and 6-bromopyridine-2,3-diamine (874 mg, 4.65 mmol, 1.0 eq), then T3P (3.25 g, 5.11 mmol, 1.1 eq, 50% purity in ethyl acetate) was added at 0° C. The mixture was stirred at 20° C. for 2 hrs. The reaction mixture was quenched by ice water (20 mL) and then extracted with ethyl acetate (40 mL*3). The combined organic phase was washed by brine (40 mL*2), dried over anhydrous sodium sulfate, filtered and the filtrate was concentrated under reduced pressure. The crude product was purified by reversed phase flash (CombiFlash 0.1% NH₃·H₂O aqueous-acetonitrile) and following up lyophilization to afford the title compound (1.0 g, 56% yield) as a brown solid. LCMS: m/z 387.1 [M+2+H]⁺, ESI pos.

### Step 2: (rac)-tert-Butyl 2-(5-bromo-1H-imidazo[4,5-b]pyridin-2-yl)pyrrolidine-1-carboxylate

To a solution of (rac)-tert-butyl 2-((2-amino-6-bromopyridin-3-yl)carbamoyl)pyrrolidine-1-carboxylate (500.0 mg, 1.3 mmol, 1.0 eq) in ethanol (5 mL) was added a solution of NaOH (779 mg, 19.5 mmol, 15 eq) in water (1 mL). The reaction was stirred at 100° C. for 4 hrs. The reaction mixture was quenched by ice water (20 mL) and extracted with ethyl acetate (20 mL*3), then washed with brine (20 mL*2), dried over anhydrous sodium sulfate, filtrated and the filtrate was concentration under reduce pressure. The residue was purified by reversed-phase flash (CombiFlash 0.1% TFA aqueous-acetonitrile condition) and following up lyophilization to afford the title compound (50 mg, 10% yield) as a light yellow solid. LCMS: m/z 367.0 [M+H]⁺, ESI pos.

### Step 3: (rac)-tert-Butyl 2-(5-(2-methoxy-6-methyl-4-(trifluoromethyl)phenyl)-1H-imidazo[4,5-b]pyridin-2-yl)pyrrolidine-1-carboxylate

A mixture of (rac)-tert-butyl 2-(5-bromo-1H-imidazo[4,5-b]pyridin-2-yl)pyrrolidine-1-carboxylate; 2, 2, 2-trifluoroacetic acid (80.0 mg, 0.170 mmol, 1.0 eq), K₂CO₃ (68.82 mg, 0.500 mmol, 3 eq) and 2-(2-methoxy-6-methyl-4-(trifluoromethyl)phenyl)-4, 4, 5, 5-tetramethyl-1, 3, 2-dioxaborolane (105.1 mg, 0.330 mmol, 2 eq) in 1, 4-dioxane (2 mL) and water (0.4 mL) was added to a round-bottom flask and purged with nitrogen three times, then Pd(dppf)Cl₂ (36.45 mg, 0.050 mmol, 0.300 eq) was added to the mixture. The reaction mixture was stirred at 90° C. for 4 hrs. The reaction mixture was filtrated and the filtrate was concentrated under reduced pressure. The residue was purified by silica gel column (PE: ethyl acetate=1:1, Rf=0.4) to afford the title compound (79 mg, 99.7% yield) as a brown solid. LCMS: m/z 477.2 [M+H]⁺, ESI pos.

### Step 4: (rac)-3-Methyl-2-(2-(pyrrolidin-2-yl)-1H-imidazo[4,5-b]pyridin-5-yl)-5-(trifluoromethyl)phenol

To a mixture of (rac)-tert-butyl 2-(5-(2-methoxy-6-methyl-4-(trifluoromethyl)phenyl)-1H-imidazo[4,5-b]pyridin-2-yl)pyrrolidine-1-carboxylate (50.0 mg, 0.10 mmol, 1.0 eq) in dichloromethane (1 mL) was added BBr₃ (66.6 mg, 0.270 mmol, 2.53 eq) under N₂. The mixture was stirred at 25° C. for 10 minutes and then stirred at 25° C. for another 50 minutes. The reaction mixture was quenched by ice water (2 mL) and neutralized by NH₃·H₂O to pH˜7. The residue was purified by reversed-phase flash (CombiFlash 0.1% TFA aqueous-acetonitrile condition) and following up lyophilization to afford the title compound (15.1 mg, 39% yield) as a white solid. LCMS: m/z 363.1 [M+H]⁺, ESI pos.

### Example 18, 18A and 18B

### (rac)-2-[2-[(1-Ethyl-3-piperidyl)amino]-1H-imidazo[4,5-b]pyridin-5-yl]-3-methyl-5-(trifluoromethyl)phenol and respective enantiomers 18A and 18B

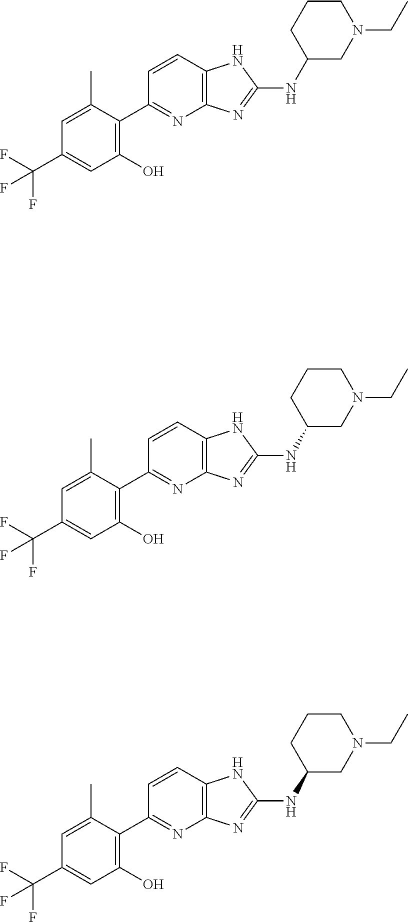 [SMILES: CCN1CCCC(Nc2nc3nc(-c4c(C)cc(C(F)(F)F)cc4O)ccc3[nH]2)C1 ; CCN1CCC[C@@H](Nc2nc3nc(-c4c(C)cc(C(F)(F)F)cc4O)ccc3[nH]2)C1 ; CCN1CCC[C@H](Nc2nc3nc(-c4c(C)cc(C(F)(F)F)cc4O)ccc3[nH]2)C1]

### Step 1: 5-Bromo-2-methylsulfanyl-1H-imidazo[4,5-b]pyridine

To a solution of (2-amino-6-bromo-3-pyridyl)amine (CAS #129012-04-0, 800 mg, 4.25 mmol, 1.0 eq) in DMF (40 mL) under argon atmosphere was added 1,1′-thiocarbonyldiimidazole (CAS #6160-65-2, 917.84 mg, 4.89 mmol, 1.15 eq) portionwise. The yellow solution changed color to black after addition. The reaction mixture was stirred at room temperature overnight. LC-MS indicated detection of intermediate so that potassium carbonate (1.18 g, 514 μL, 8.51 mmol, 2.0 eq) was added followed by methyl iodide (307.25 μL, 4.94 mmol, 1.16 eq). The reaction mixture was stirred at room temperature for 2 h30. After complete conversion (reaction control using TLC 1:1 ethyl acetate/hexane). The reaction mixture was cooled to 0° C. and ˜25 mL water was added dropwise. The reaction mixture was diluted with further water (25 mL) and extracted 3 times with ethyl acetate (20 mL). The organic layers were washed with water and brine, dried over Na₂SO₄ and concentrated under reduced pressure. The residue was triturated with ether to afford the title compound (618 mg, 60% yield) as a light brown powder. LCMS: m/z 244.0 [M+H]⁺ (Br), ESI pos.

### Step 2: (rac)-(5-Bromo-N-(1-ethyl-3-piperidyl)-1H-imidazo[4,5-b]pyridin-2-amine

In a sealed tube, to a mixture of aforementioned 5-bromo-2-methylsulfanyl-1H-imidazo[4,5-b]pyridine (Example 18, step 1) (200 mg, 0.819 mmol, 1.0 eq) was added (rac)-(1-ethyl-3-piperidyl)amine (CAS #6789-94-2, 552.9 mg, 615.7 μL, 4.1 mmol, 5.0 eq). The light yellow suspension was stirred at 150° C. for 4 d and one additional day at 130° C. After reaction completion, the reaction mixture was cooled to room temperature. The crude product was adsorbed on ISOLUTE HM-N and purified by flash chromatography (silica gel, 15 g, gradient 0% to 10% methanol in dichloromethane) to afford the title compound (187 mg, 56%, 80% purity) as viscous oil. LCMS: m/z 324.2 [M+H]⁺, ESI pos.

### Step 3: (rac)-2-[2-[(1-Ethyl-3-piperidyl)amino]-1H-imidazo[4,5-b]pyridin-5-yl]-3-methyl-5-(trifluoromethyl)phenol

To a solution of aforementioned (rac)-(5-Bromo-1H-imidazo[4,5-b]pyridin-2-yl)-(1-ethyl-3-piperidyl)amine (Example 18, step 2) (196.8 mg, 0.577 mmol, 1.0 eq) in 1,4-dioxane (3.3 mL) and water (1.65 mL) was added (2-hydroxy-6-methyl-4-(trifluoromethyl)phenyl)boronic acid (314.8 mg, 1.43 mmol, 2.48 eq), potassium carbonate (559 mg, 4.04 mmol, 7.0 eq) and Pd(dppf)Cl₂ (79.1 mg, 0.097 mmol, 0.17 eq). The reaction mixture was stirred at 85° C. for 6 hrs under N₂ atmosphere. The reaction mixture was cooled to room temperature and extracted with ˜30 mL ethyl acetate and ˜4 mL half-saturated NH₄Cl-solution. The aqueous layer was backextracted with ˜30 mL ethyl acetate. The organic layers were washed with ˜4 mL water and ˜4 mL brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (110 mg, 46%) as an light brown powder. LCMS: m/z 418.4 [M+H]⁺, ESI pos.

### Step 4: 2-[2-[[(3R or 3S)-1-Ethyl-3-piperidyl]amino]-1H-imidazo[4,5-b]pyridin-5-yl]-3-methyl-5-(trifluoromethyl)phenol and 2-[2-[[(3S or 3R)-1-Ethyl-3-piperidyl]amino]-1H-imidazo[4,5-b]pyridin-5-yl]-3-methyl-5-(trifluoromethyl)phenol

Aforementioned (Example 18, step 3) (rac)-2-[2-[(1-Ethyl-3-piperidyl)amino]-1H-imidazo[4,5-b]pyridin-5-yl]-3-methyl-5-(trifluoromethyl)phenol was separated by chiral SFC (column: chiral cellulose C4, 5 μm, 250×20 mm), eluent: methanol 35%+0.2% diethylamine) to afford the two enantiomers example 18A (first eluting, Rt=2.90 min, optical rotation (in MeOH, 20° C.): +19.44°) (33.0 mg, 27% yield, 90% purity, contains diethylamine) as a white powder; m/z 418.4 [M+H]⁺, ESI pos and example 18B (second eluting, Rt=4.12 min) (28.0 mg, 25% yield) as an white powder. LCMS: m/z 418.4 [M+H]⁺, ESI pos.

### Example 19

### 5-Chloro-2-[2-[[(3R)-1-ethyl-3-piperidyl]amino]oxazolo[4,5-b]pyridin-5-yl]-3-methyl-phenol

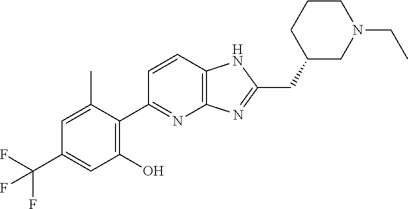 [SMILES: CCN1CCC[C@@H](Cc2nc3nc(-c4c(C)cc(C(F)(F)F)cc4O)ccc3[nH]2)C1]

A mixture of 5-chloro-N-[(3R)-1-ethyl-3-piperidyl]oxazolo[4,5-b]pyridin-2-amine (Example 14, step 1) (450 mg, 1.52 mmol, 1.00 eq), (4-chloro-2-hydroxy-6-methyl-phenyl)boronic acid (CAS #1207961-50-9, 480 mg, 2.58 mmol, 1.69 eq), potassium carbonate (1.01 g, 7.31 mmol, 4.80 eq) and 1,1′-bis(diphenylphosphino)ferrocene-palladium(ii) dichloride dichloromethane complex (189 mg, 0.23 mmol, 0.15 eq) in 1,4-dioxane (9.0 mL) and water (4.5 mL) was flushed with argon and stirred at 95° C. for 16 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 40 g, gradient 0% to 10% methanol in dichloromethane). All fractions containing product were combined and concentrated in vacuo to afford the title compound (414 mg, 67% yield) as a dark brown solid. LC-MS: m/z 387.2 [M+H]⁺, ESI pos.

### Example 20, 20A and 20B

### (rac)-5-Chloro-3-methyl-2-[2-(6-methyl-3,3a,4,5,7,7a-hexahydro-2H-pyrrolo[2,3-c]pyridin-1-yl)oxazolo[4,5-b]pyridin-5-yl]phenol and enantiomers 20A and 20B

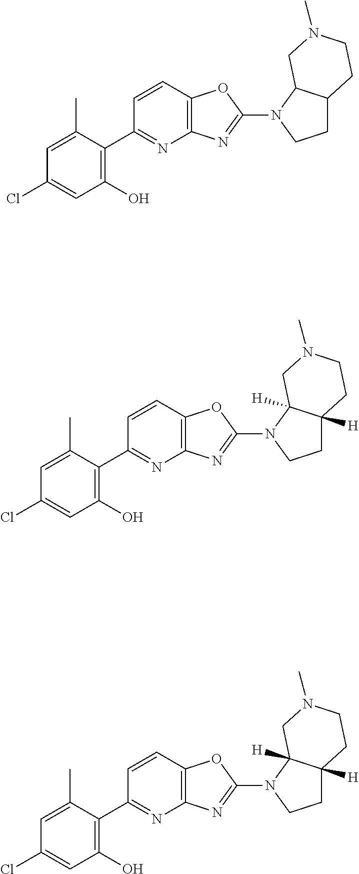 [SMILES: Cc1cc(Cl)cc(O)c1-c1ccc2oc(N3CCC4CCN(C)CC43)nc2n1 ; Cc1cc(Cl)cc(O)c1-c1ccc2oc(N3CC[C@H]4CCN(C)C[C@@H]43)nc2n1 ; Cc1cc(Cl)cc(O)c1-c1ccc2oc(N3CC[C@H]4CCN(C)C[C@H]43)nc2n1]

### Step 1: (rac)-5-Chloro-2-(6-methyl-3,3a,4,5,7,7a-hexahydro-2H-pyrrolo[2,3-c]pyridin-1-yl)oxazolo[4,5-b]pyridine

In a sealed tube, triethylamine (374 mg, 515 μL, 3.69 mmol, 3.0 eq) was added under stirring at rt to a mixture of 5-chloro-2-(methylthio)oxazolo[4,5-b]pyridine (CAS #1783370-92-2, 260 mg, 1.23 mmol, 1.0 eq) and 6-methyloctahydro-1H-pyrrolo[2,3-c]pyridine (CAS #1443980-22-0, 224 mg, 1.6 mmol, 1.3 eq) in 1,4-dioxane (4 mL) and N-methyl-2-pyrrolidinone (2 mL). The reaction mixture was heated at 120° C. for 2 hours. Afterwards the dark-brown reaction mixture was heated at 150° C. for 20 hours. The black reaction mixture was cooled to rt and extracted twice with ethyl acetate:tBME (v/v) 1:1 (2×50 mL). The organic layers were washed with water (20 mL) and brine (20 mL). The aqueous layers were back extracted with ethyl acetate:tBME (v/v) 1:1 (50 mL). The combined organic phases were dried over sodium sulfate, filtered and concentrated in vacuo. The crude product was absorbed on ISOLUTE HM-N and purified by flash chromatography (12 g SiO₂; gradient 0%-10% methanol in dichlormethane) to afford the title compound (313 mg, 86% yield) as yellow solid. LC-MS: m/z 293.1 [M+H]⁺, ESI pos.

### Step 2: (rac)-5-Chloro-3-methyl-2-[2-(6-methyl-3,3a,4,5,7,7a-hexahydro-2H-pyrrolo[2,3-c]pyridin-1-yl)oxazolo[4,5-b]pyridin-5-yl]phenol

5-Chloro-2-(6-methyl-3,3a,4,5,7,7a-hexahydro-2H-pyrrolo[2,3-c]pyridin-1-yl)oxazolo[4,5-b]pyridine (112 mg, 0.379 mmol, 1.0 eq) and (4-chloro-2-hydroxy-6-methyl-phenyl)boronic acid (141.2 mg, 0.757 mmol, 2.00 eq) were dissolved in 1,4-dioxane (3 mL) and water (1.5 mL). Then potassium carbonate (235.6 mg, 1.7 mmol, 4.5 eq) was added at rt. The orange reaction mixture was purged with argon and 1,1′-bis(diphenylphosphino)ferrocene-palladium(ii)dichloride dichloromethane complex (61.9 mg, 0.076 mmol, 0.2 eq) was added. The tube was sealed and stirred at 100° C. for 20 h. Then the reaction was cooled to rt and (4-chloro-2-hydroxy-6-methyl-phenyl)boronic acid (70.6 mg, 0.379 mmol, 1.0 eq) in 1,4-dioxane (1.5 mL) and water (0.750 mL) were added followed by the addition of potassium carbonate (235.6 mg, 1.7 mmol, 4.5 eq). The dark-red reaction mixture was purged with argon and 1,1′-bis(diphenylphosphino)ferrocene-palladium(ii)dichloride dichloromethane complex (30.9 mg, 0.038 mmol, 0.1 eq) was added under stirring. The tube was sealed and stirred at 110° C. was continued for 6 h. Afterwards, the reaction mixture was extracted twice with DCM (2×100 mL) and aq. ammonium chloride solution (50 mL). The organic layers were washed with water (50 mL) and brine (50 mL). The combined organic extracts were dried over sodium sulfate, filtered and the solvent was evaporated under reduced pressure. The crude product was absorbed on ISOLUTE HM-N and purified by flash chromatography (SiO2; 0%-30% dichlormethane:methanol:NH4OH (v/v) 110:10:1 in dichlormethane) followed by further purification by preparative HPLC to the title compound (30.2 mg, 18% yield) as white powder. LC-MS: m/z 397.1 [M−H]⁻, ESI neg.

### Step 3: 2-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-5-chloro-3-methyl-phenol and 2-[2-[(3aS,7aR or 3aR,7aS)-6-methyl-3,3a,4,5,7,7a-hexahydro-2H-pyrrolo[2,3-c]pyridin-1-yl]oxazolo[4,5-b]pyridin-5-yl]-5-chloro-3-methyl-phenol

Aforementioned (rac)-5-Chloro-3-methyl-2-[2-(6-methyl-3,3a,4,5,7,7a-hexahydro-2H-pyrrolo-[2,3-c]pyridin-1-yl)oxazolo[4,5-b]pyridin-5-yl]phenol (Example 20, step 2) was separated by chiral SFC (column: Column chiral IJ, 5 μm, 250×20 mm), eluent: methanol 25%+0.2% diethylamine) to afford the two enantiomers example 20A (8 mg, 41% yield, first eluting, Rt=1.46 min) as a light brown foam; m/z 399.2 [M+H]⁺, ESI pos and example 20B (8 mg, 41% yield, second eluting, Rt=2.26 min) m/z 399.2 [M+H]⁺, ESI pos as a light brown foam.

### Example 21

### 5-Chloro-2-[2-[[(3R)-1-ethyl-3-piperidyl]amino]oxazolo[4,5-b]pyridin-5-yl]-6-fluoro-3-methyl-phenol;2,2,2-trifluoroacetic acid salt

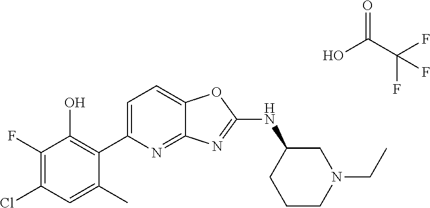 [SMILES: CCN1CCC[C@@H](Nc2nc3nc(-c4c(C)cc(Cl)c(F)c4O)ccc3o2)C1 ; O=C(O)C(F)(F)F]

### Step A: 2-(3-Chloro-2-fluoro-5-methylphenyl)-4,4,5,5-tetramethyl-1,3,2-dioxaborolane

To a solution of 4,4,4′,4′,5,5,5′,5′-octamethyl-2,2′-bi(1,3,2-dioxaborolane) (4391.3 mg, 17.3 mmol, 1.0 eq) in THF (50 mL) was added 4,4′-di-tert-butyl-2,2′-bipyridine (139.2 mg, 0.52 mmol, 0.03 eq) and (1,5-cyclooctadiene)(methoxy)iridium(I) dimer (229.25 mg, 0.35 mmol, 0.02 eq), then 2-chloro-1-fluoro-4-methylbenzene (2500.0 mg, 17.29 mmol, 1.0 eq) was added. The above reaction mixture was stirred at 80° C. for 12 hours. Then, reaction mixture was cooled to room temperature, diluted with water (200 mL), extracted with ethyl acetate (100 mL×3). The combined organic phase was washed with brine (100 mL), dried over anhydrous sodium sulfate, filtered and the filtrate was concentrated under reduced pressure. The residue was purified by silica gel column (petroleum ether:ethyl acetate=1:0 to 20:1) to afford the title compound (4.5 g, 96% yield) as a yellow solid. ¹H NMR (400 MHz, DMSO-d₆) δ 7.50 (dd, 1H), 7.36 (dd, 1H), 2.27 (s, 3H), 1.29 (s, 12H).

### Step B: 3-Chloro-2-fluoro-5-methylphenol

To a solution of 2-(3-chloro-2-fluoro-5-methylphenyl)-4,4,5,5-tetramethyl-1,3,2-dioxaborolane (4.50 g, 16.6 mmol, 1.0 eq) in THF (50 mL) was slowly dropped H₂O₂ (9.43 g, 83.17 mmol, 5.0 eq) at 0° C., then stirred at 20° C. for 2 hours. The above reaction solution was diluted with water (50 mL), then Na₂S203 (16 g, 83 mmol) was added, stirred at 20° C., for 20 min, extracted with ethyl acetate (100 mL×3). The combined organic phase was washed with brine (100 mL), dried over anhydrous sodium sulfate, filtered and the filtrate was concentrated under reduced pressure. The crude product was purified by silica gel column (petroleum ether:ethyl acetate=1:0 to 20:1) to afford the title compound (2500 mg, 84% yield) as yellow oil. ¹H NMR (400 MHz, DMSO-d₆) δ 10.19 (br.s, 1H), 6.77-6.68 (m, 2H), 2.18 (s, 3H).

### Step C: 3-Chloro-2-fluoro-6-iodo-5-methylphenol

To a solution of 3-chloro-2-fluoro-5-methylphenol (1.50 g, 9.34 mmol, 1.0 eq) in toluene (30 mL) was added NaH (934.2 mg, 23.35 mmol, 60% purity in mineral oil, 2.5 eq) in portions at 0° C., then stirred at 20° C. for 0.5 hour, then iodine (2.13 g, 8.41 mmol, 0.9 eq) was slowly added at 0° C. in portions. The above reaction mixture was stirred at 20° C. for 0.5 hour. The above reaction mixture was quenched with diluted hydrochloric acid (100 mL, 1 M solution of water) at 0° C., extracted with ethyl acetate (50 mL×3). The combined organic phase was washed with brine (100 mL), dried over anhydrous sodium sulfate, filtered and the filtrate was concentrated under reduced pressure. The crude product was purified by silica gel column (petroleum ether:ethyl acetate=1:0 to 10:1) to afford the title compound (1.10 g, 41% yield) as yellow oil. LC-MS: m/z 284.8 [M−H]⁻, ESI neg.

### Step D: 1-Chloro-2-fluoro-4-iodo-3-methoxy-5-methylbenzene

To a solution of 3-chloro-2-fluoro-6-iodo-5-methylphenol (1.0 g, 3.49 mmol, 1.0 eq) in DMF (5 mL) was added K₂CO₃ (1206.06 mg, 8.73 mmol, 2.5 eq), then Mel (0.43 mL, 6.98 mmol, 2.0 eq) was dropped at 0° C., then stirred at 20° C. for 2 hours. The above reaction solution was diluted with water (100 mL), extracted with ethyl acetate (50 mL×3). The combined organic phase was washed with brine (100 mL×3), dried over anhydrous sodium sulfate, filtered and the filtrate was concentrated under reduced pressure The crude product was purified by column chromatography (silica gel, petroleum ether:ethyl acetate=1:0 to 10:1) to the title compound (700.0 mg, 67% yield) as yellow oil. ¹H NMR (400 MHz, CDCl₃) δ 7.08 (dd, 1H), 3.95 (d, 3H), 2.42 (s, 3H).

### Step E: 2-(4-Chloro-3-fluoro-2-methoxy-6-methylphenyl)-4,4,5,5-tetramethyl-1,3,2-dioxaborolane

To a solution of 2-isopropoxy-4,4,5,5-tetramethyl-1,3,2-dioxaborolane (557.3 mg, 3.0 mmol, 1.5 eq) in THF (12 mL) was added 1-chloro-2-fluoro-4-iodo-3-methoxy-5-methylbenzene (600.0 mg, 2.0 mmol, 1.0 eq), and then n-BuLi (1.5 mL, 3.75 mmol, 2.5 M in hexane, 1.88 eq) was dropped at −70° C., then stirred at −70° C. for 0.5 hour under N2. The above reaction solution was quenched with saturated ammonium chloride (100 mL) at 0° C., extracted with ethyl acetate (50 mL×3). The combined organic phase was washed with brine (100 mL), dried over anhydrous sodium sulfate, filtered and the filtrate was concentrated under reduced pressure. The crude product was purified by column chromatography (silica gel, petroleum ether:ethyl acetate=1:0 to 2:1) to afford the title compound (400.0 mg, 67% yield) as colorless oil. ¹H NMR (400 MHz, CDCl₃) δ 6.89 (d, 1H), 3.91 (d, 3H), 2.30 (s, 3H), 1.39 (s, 12H).

### Step F: (4-Chloro-3-fluoro-2-hydroxy-6-methylphenyl)boronic acid

To a solution of 2-(4-chloro-3-fluoro-2-methoxy-6-methylphenyl)-4,4,5,5-tetramethyl-1,3,2-dioxaborolane (150.0 mg, 0.5 mmol, 1.0 eq) in DCM (2 mL) was dropwise added BBr₃ (0.5 mL, 4.99 mmol, 10.0 eq) under N₂ at −60° C., and then the mixture was stirred at 25° C. for 2 hours. The reaction mixture was poured into 10 mL water at 0° C., The pH was adjusted to 8 with 1M NaOH solution and the mixture was then washed with ethyl acetate (10 mL×3), then the aqueous phase was acidified with 1 N HCl solution to pH=4. extracted with ethyl acetate (20 mL×3). The combined organic phase was washed with brine (50 mL), dried over anhydrous sodium sulfate, filtered and the filtrate was concentrated under reduced pressure to afford (4-chloro-3-fluoro-2-hydroxy-6-methylphenyl)boronic acid (40 mg, 39% yield) as yellow oil. ¹H NMR (400 MHz, CD₃OD) δ 6.73 (d, 1H), 2.17 (s, 3H).

### Step G: 5-Bromo-2-methylsulfanyl-oxazolo[4,5-b]pyridine

1,1′-Thiocarbonyldiimidazole (5.42 g, 30.42 mmol, 1.15 eq) was added portion-wise to a stirred solution of 2-amino-6-bromopyridin-3-ol (CAS #934758-27-7, 5.0 g, 26.45 mmol, 1.0 eq,) in DMF (100 mL) at rt. The reaction mixture was stirred under nitrogen for 16 h. Potassium carbonate (7.31 g, 52.91 mmol, 2.0 eq) followed by iodomethane (1.9 mL, 30.52 mmol, 1.15 eq) were then added and the reaction was stirred for a further 18 h. The reaction mixture was cooled to 0° C. and quenched by the slow addition of water (70 mL) upon which a precipitate formed. The reaction mixture was filtered and the solid washed with water. The product was dissolved in MeCN and concentrated in vacuo after which the product was dried in a vacuum oven overnight to afford the title compound (4.89 g, 68% yield) as a brown solid. LC-MS: m/z 244.9/246.9 [M+H]⁺, ESI pos.

### Step H: 5-Bromo-N-[(3R)-1-ethyl-3-piperidyl]oxazolo[4,5-b]pyridin-2-amine

Triethylamine (3.3 mL, 23.7 mmol, 5.0 eq), (3R)-1-ethylpiperidin-3-amine dihydrochloride (1.9 g, 9.47 mmol, 2.0 eq) and 5-bromo-2-methylsulfanyl-oxazolo[4,5-b]pyridine (CAS #1149384-63-3, 1.16 g, 4.73 mmol, 1.0 eq) were dissolved in dioxane (20 mL) and the reaction mixture stirred at 90° C. for 24 h. The reaction mixture was diluted with water (50 mL) and extracted with EtOAc (2×50 mL). The combined organic extracts were dried using a phase separator and concentrated in vacuo. The resulting residue was purified by column chromatography (silica gel, 40 g, 0-10% (0.7 N NH₃ in MeOH)/DCM) to afford the title compound (1.61 g, 91% yield) as a brown semisolid. LC-MS: m/z 325.2/327.2 [M+H]⁺, ESI pos.

### Step I: 5-Chloro-2-[2-[[(3R)-1-ethyl-3-piperidyl]amino]oxazolo[4,5-b]pyridin-5-yl]-6-fluoro-3-methyl-phenol;2,2,2-trifluoroacetic acid salt

To a solution of (4-chloro-3-fluoro-2-hydroxy-6-methylphenyl)boronic acid (40 mg, 0.2 mmol, 1.0 eq) in 1,4-dioxane (2 mL) and water (0.4 mL) was added 5-bromo-N-[(3R)-1-ethyl-3-piperidyl]oxazolo[4,5-b]pyridin-2-amine;2,2,2-trifluoroacetic acid (68.77 mg, 0.16 mmol, 0.8 eq), K₂CO₃ (67.6 mg, 0.49 mmol, 2.5 eq) and Pd(dppf)Cl₂ (28.64 mg, 0.04 mmol, 0.2 eq), then stirred at 95° C. for 2 hours under N₂ atmosphere. The above reaction mixture was cooled to room temperature and diluted with acetonitrile (5 mL), filtered and the filtrate was concentrated under reduced pressure. The crude product was purified by reversed phase chromatography (0.1% TFA, water-MeCN), then the solvent was removed by lyophilization to afford the title compound (13.9 mg, 13% yield) as a white solid. LC-MS: m/z 404.9 [M−H]⁻, ESI neg.

### Example 22

### 5-Chloro-2-[2-[[(3R)-1-ethyl-3-piperidyl]amino]oxazolo[4,5-b]pyridin-5-yl]-4-fluoro-3-methyl-phenol;2,2,2-trifluoroacetic acid salt

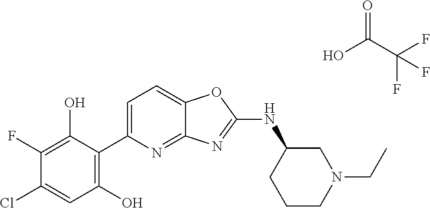 [SMILES: CCN1CCC[C@@H](Nc2nc3nc(-c4c(O)cc(Cl)c(F)c4O)ccc3o2)C1 ; O=C(O)C(F)(F)F]

### Step A: 6-Bromo-4-chloro-3-fluoro-2-methylaniline

To a solution of 4-chloro-3-fluoro-2-methylaniline (4.50 g, 28.2 mmol, 1.0 eq) in MeCN (100 mL) was added NBS (6.02 g, 33.8 mmol, 1.2 eq) in one portion, The mixture was stirred at 20° C. for 2 hours under a nitrogen atmosphere. The mixture was diluted with 50 mL of water, extracted with ethyl acetate (40 mL*3), the combined organic layer dried over anhydrous sodium sulfate, filtered and concentrated under reduced pressure. The residue was purified by silica gel column (petroleum ether:ethyl acetate=1:0 to 10:1) to afford the title compound (5.00 g, 74% yield) as a dark brown solid. LC-MS: m/z 238.0 [M+H]⁺, ESI pos.

### Step B: 4-Chloro-3-fluoro-6-methoxy-2-methylaniline

To a solution of 6-bromo-4-chloro-3-fluoro-2-methylaniline (3.00 g, 12.6 mmol, 1.0 eq) and CuI (2.38 g, 12.6 mmol, 1.0 eq) in DMF (60 mL) was added NaOMe (9.06 g, 50.32 mmol, 4.0 eq) slowly at 20° C. The mixture was stirred at 120° C. for 3 hours. The above reaction mixture was cooled to room temperature, saturated ammonium chloride (200 mL) was added and extracted with ethyl acetate (200 mL*3). The combined organic extracts were dried with anhydrous sodium sulfate and concentrated under reduced pressure. The crude product was purified by silica gel column (petroleum ether:ethyl acetate=20:1 to 5:1) to afford the title compound (900 mg, 38% yield) as a dark brown solid. LC-MS: m/z 190.1 [M+H]⁺, ESI pos.

### Step C: 2-Bromo-5-chloro-4-fluoro-1-methoxy-3-methylbenzene

To a solution of 4-chloro-3-fluoro-6-methoxy-2-methylaniline (250.0 mg, 1.32 mmol, 1.0 eq), CuBr (37.83 mg, 0.26 mmol, 0.2 eq) and CuBr₂ (294.5 mg, 1.32 mmol, 1.0 eq) in MeCN (5 mL) was added tert-butylnitrite (314 μL, 2.64 mmol, 2.0 eq) slowly at 0° C. The mixture was stirred at 20° C. for 2 hours under nitrogen. The above reaction mixture was diluted with water (20 mL), extracted with ethyl acetate (20 mL*3), then combined organic extracts were dried over anhydrous sodium sulfate and concentrated under reduced pressure. The crude product was purified by column chromatography (silica gel, petroleum ether:ethyl acetate=1:0 to 10:1) to afford the title compound (150.0 mg, 45% yield) as a white solid. ¹H NMR (400 MHz, DMSO-d) δ 7.23 (d, 1H), 3.86 (s, 3H), 2.32 (d, 3H).

### Step D: 2-(4-Chloro-3-fluoro-6-methoxy-2-methylphenyl)-4,4,5,5-tetramethyl-1,3,2-dioxaborolane

To a solution of 2-bromo-5-chloro-4-fluoro-1-methoxy-3-methylbenzene (50.0 mg, 0.2 mmol, 1.0 eq) in THF (1 mL) was added 2-isopropoxy-4,4,5,5-tetramethyl-1,3,2-dioxaborolane (91.8 mg, 0.49 mmol, 2.5 eq), then n-BuLi (0.12 mL, 0.3 mmol, 2.5 M in hexane, 1.5 eq) was dropped under −70° C., then stirred at −70° C. for 1 hour. The above reaction solution was quenched with saturated ammonium chloride solution (50 mL) at 0° C., extracted with ethyl acetate (20 mL*3). The combined organic phase was washed with brine (50 mL), dried over anhydrous sodium sulfate, filtered and the filtrate was concentrated under reduced pressure. The crude product was purified by prep-TLC (petroleum ether:ethyl acetate=20:1, Rf=0.4) to afford the title compound (40.0 mg, 67% yield) as a white solid. ¹H NMR (400 MHz, CDCl₃) δ 6.66 (d, 1H), 3.73 (s, 3H), 2.28 (d, 3H), 1.41 (s, 12H).

### Step E: (R)-5-(4-Chloro-3-fluoro-6-methoxy-2-methylphenyl)-N-(1-ethylpiperidin-3-yl)oxazolo[4,5-b]pyridin-2-amine

To a solution of 2-(4-chloro-3-fluoro-6-methoxy-2-methylphenyl)-4,4,5,5-tetra methyl-1,3,2-dioxaborolane (30.0 mg, 0.1 mmol, 1.0 eq) in 1,4-dioxane (1 mL) and water (0.2 mL) was added (R)-6-bromo-N-(1-ethylpiperidin-3-yl)-3H-pyrrolo[2,3-b]Pyridin-2-amine (43.8 mg, 0.1 mmol, 1.0 eq), CsF (37.9 mg, 0.25 mmol, 2.5 eq) and Xphos Pd G3 (16.92 mg, 0.02 mmol, 0.2 eq), then the above reaction mixture was stirred at 90° C. for 1 hour under N₂ atmosphere. The above reaction solution was diluted with water (100 mL), extracted with ethyl acetate (20 mL*3). The combined organic phase was washed with brine (50 mL), dried over anhydrous sodium sulfate, filtered and the filtrate was concentrated under reduced pressure. The crude product was purified by prep-TLC (dichloromethane:methanol=10:1, Rf=0.1) to afford the title compound (40.0 mg, 63% yield) as a yellow oil. LC-MS: m/z 419.0 [M+H]⁺, ESI pos.

### Step F: 5-Chloro-2-[2-[[(3R)-1-ethyl-3-piperidyl]amino]oxazolo[4,5-b]pyridin-5-yl]-4-fluoro-3-methyl-phenol;2,2,2-trifluoroacetic acid salt

To a solution of (R)-5-(4-chloro-3-fluoro-6-methoxy-2-methylphenyl)-N-(1-ethylpiperidin-3-yl)oxazolo[4,5-b]pyridin-2-amine (30.0 mg, 0.07 mmol, 1.0 eq) in dichloromethane (1 mL) was added BBr₃ (178.8 mg, 0.72 mmol, 10.0 eq) at −20° C., then stirred at 20° C. for 1 hour. The above reaction mixture was quenched with water (0.5 mL) at 0° C., diluted with methanol (1 ml), then the pH was adjusted to about 7 with ammonium hydroxide, then purified by reversed phase chromatography (C18, 0.1% TFA, water-ACN), then the solvent was removed by lyophilization to afford the title compound (10.6 mg, 28% yield) as a white solid. LC-MS: m/z 405.0 [M+H]⁺, ESI pos.

### Example 23

### 5-Chloro-2-[2-[[(3R)-1-ethyl-3-piperidyl]amino]oxazolo[4,5-b]pyridin-5-yl]-3-hydroxy-benzonitrile

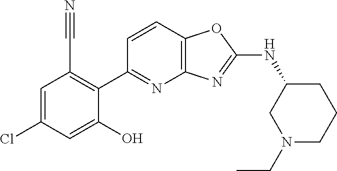 [SMILES: CCN1CCC[C@@H](Nc2nc3nc(-c4c(O)cc(Cl)cc4C#N)ccc3o2)C1]

### Step A: 2-Bromo-5-chloro-3-hydroxy-benzonitrile

N-Bromosuccinimide (3.60 g, 20.2 mmol, 1.04 eq) was added in portions to a stirred solution of 3-chloro-5-hydroxybenzonitrile (CAS #473923-97-6, 3.0 g, 19.5 mmol, 1.0 eq) and diisopropylamine (1.0 mL, 7.14 mmol, 0.37 eq) in DCM (50 mL) at 0° C. and the reaction was allowed to return to rt and was stirred for 3 days. The solvent was concentrated in vacuo and the crude material was purified by column chromatography (silica gel, 330 g, DCM) to give the title compound (738 mg, 16% yield) as a white solid. LC-MS: m/z 229.9 [M−H]⁻, ESI neg.

### Step B: 2-Bromo-5-chloro-3-methoxy-benzonitrile

Potassium carbonate (875.0 mg, 6.33 mmol, 2.02 eq) was added to a stirred solution of 2-bromo-5-chloro-3-hydroxy-benzonitrile (730.0 mg, 3.14 mmol, 1.0 eq) in acetone (8 mL) at rt and the reaction was stirred for 5 mins. Iodomethane (300.0 μL, 4.82 mmol, 1.53 eq) was then added and the reaction was stirred for a further 16 h. The reaction was concentrated in vacuo, then diluted with DCM (50 mL) and water (50 mL). The organic layer was separated and the aqueous was extracted again with DCM (2×50 mL). The combined organic layers were dried with MgSO₄ and concentrated in vacuo to give the title compound (752.0 mg, 92% yield) as a white solid which was used without further purification. LC-MS no m/z observed. ¹H NMR (500 MHz, DMSO-d₆) δ 7.72 (d, 1H), 7.57 (d, 1H), 3.95 (s, 3H).

### Step C: 5-Chloro-3-methoxy-2-(4.4,5,5-tetramethyl-1,3,2-dioxaborolan-2-yl)benzonitrile

Isopropylmagnesium chloride LiCl complex (1.3M solution) (5.85 mL, 7.61 mmol, 2.5 eq) was added dropwise to a stirred solution of 2-bromo-5-chloro-3-methoxy-benzonitrile (750.0 mg, 3.04 mmol, 1.0 eq) and 2-isopropoxy-4,4,5,5-tetramethyl-1,3,2-dioxaborolane (3.1 mL, 15.21 mmol, 5.0 eq) in THF (30 mL) at 0° C. The reaction was allowed to return to rt and was stirred for 16 h. The reaction mixture was concentrated in vacuo, then was diluted with water (100 mL) and DCM (100 mL) and the organic layer was separated. The aqueous layer was extracted again with DCM (2×100 mL) and the combined organic layers were washed with water (2×100 mL) and brine (100 mL), then were dried using MgSO₄ and concentrated in vacuo to give the title compound (914.6 mg, 72% yield) as a light yellow waxy solid. LC-MS m/z 210.2 [M−BPin]⁻, ESI neg.

### Step D: 5-Chloro-2-[2-[[(3R)-1-ethyl-3-piperidyl]amino]oxazolo[4,5-b]pyridin-5-yl]-3-methoxy-benzonitrile

A mixture of 5-bromo-N-[(3R)-1-ethyl-3-piperidyl]oxazolo[4,5-b]pyridin-2-amine (Example 21, step H) (200 mg, 0.62 mmol, 0.96 eq), 5-chloro-3-methoxy-2-(4,4,5,5-tetramethyl-1,3,2-dioxaborolan-2-yl)benzonitrile (250.0 mg, 0.64 mmol, 1.0 eq), cesium carbonate (620.0 mg, 1.9 mmol, 2.98 eq) and Xphos Pd G3 (55.0 mg, 0.06 mmol, 0.1 eq) in 1,4-dioxane (4.5 mL) and water (0.5 mL) was degassed with nitrogen for 5 mins, then was heated to 90° C. for 1 h. The reaction was allowed to cool to rt, concentrated and then was purified by column chromatography on silica gel (40 g, 0-10% MeOH (0.7M NH₃)/DCM) to give the title compound (131 mg, 17% yield) as a light yellow solid. LC-MS m/z 412.3 [M+H]⁺, ESI pos.

### Step E: 5-Chloro-2-[2-[[(3R)-1-ethyl-3-piperidyl]amino]oxazolo[4,5-b]pyridin-5-yl]-3-hydroxy-benzonitrile

BBr₃ (1M in DCM) (0.98 mL, 0.98 mmol, 10.23 eq) was added dropwise to a stirred solution of 5-chloro-2-[2-[[(3R)-1-ethyl-3-piperidyl]amino]oxazolo[4,5-b]pyridin-5-yl]-3-methoxy-benzonitrile (131.0 mg, 0.1 mmol, 1.0 eq) and tetrabutylammonium iodide (123.34 mg, 0.33 mmol, 3.5 eq) in DCM (5 mL) at 0° C. and the reaction was allowed to return to rt. After 24 h, the reaction was chilled to 0° C. and a further equivalent of BBr₃ (1M in DCM) (0.98 mL, 0.98 mmol, 10.23 eq) was added. The reaction was allowed to return to rt, then was stirred for another 24 h. The reaction was quenched by dropwise addition to a stirred solution of NH₃ in MeOH (100 mL, 7M) at 0° C. The solvent was concentrated in vacuo, and the resulting residue was taken up in DCM (20 mL) and water (20 mL). The aqueous phase was adjusted to ˜pH 12 with aqueous NaOH (2M) and the organic phase was separated. The aqueous phase was extracted again with DCM (2×20 mL). The combined organic layers were dried with MgSO₄, concentrated in vacuo and then purified by column chromatography on silica gel (40 g cartridge, 0-5% MeOH (7M NH₃)/DCM). The product was then taken up in DCM (5 mL) and washed with water (5×5 mL), then was dried by passing through a phase separator and concentrated in vacuo to afford the title compound (5.0 mg, 12% yield) as an off-white solid. LC-MS m/z 398.3 [M+H]⁺, ESI pos, m/z 396.2 [M−H]⁻, ESI neg.

### Example 24

### 5-Chloro-2-[2-[[(3R)-1-ethyl-3-piperidyl]amino]oxazolo[4,5-b]pyridin-5-yl]-3-(methoxymethyl)phenol;2,2,2-trifluoroacetic acid salt

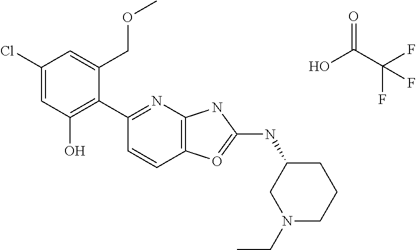

### Step A:_5-Chloro-3-hydroxy-2-iodo-benzaldehyde

To a mixture of 3-chloro-5-hydroxybenzaldehyde (7.5 g, 47.9 mmol, 1.0 eq) in DMF (80 mL) was added NaH (2299.3 mg, 95.8 mmol, 2.0 eq) at 0° C. under N₂, stirred for 30 mins, then I₂ (12157.98 mg, 47.9 mmol, 1.0 eq) was added portionwise and stirred for 15.5 hours at 25° C. The mixture was poured into water (150 mL) and extracted with ethyl acetate (100 mL×3). The combined organic layers were washed with brine (150 mL), dried over anhydrous sodium sulfate, filtered, and the filtrate was concentrated under reduced pressure. Then, the mixture purified by column chromatography (SiO₂, Petroleum ether/Ethyl acetate=1/0 to 10/1) and purified by preparative HPLC (NH₃—H₂O) and lyophilized to give the 5-chloro-3-hydroxy-2-iodo-benzaldehyde (1300 mg, 10% yield) as light green solid. ¹H NMR (400 MHz, DMSO-d6) δ 11.45-11.27 (m, 1H), 10.03-9.98 (m, 1H), 7.21-7.19 (m, 1H), 7.17-7.14 (m, 1H)

### Step B: 5-Chloro-2-iodo-3-(2-trimethylsilylethoxymethoxy)benzaldehyde

A mixture of Cs₂CO₃ (2249.34 mg, 6.9 mmol, 1.5 eq) and 5-chloro-3-hydroxy-2-iodo-benzaldehyde (1300.0 mg, 4.6 mmol, 1.0 eq) in DMF (2 mL) was degassed and purged with N₂ three times and stirred for 30 min. Then, SEMCl (1150.97 mg, 6.9 mmol, 1.5 eq) was added to the mixture in one portion. The mixture was stirred at 25° C. for 2 h. The mixture was poured into water (50 mL) and extracted with ethyl acetate (40 mL×3). The combined organic layers were washed with brine (20 mL), dried over anhydrous sodium sulfate, filtered, and the filtrate was concentrated under reduced pressure to give the title compound (1200 mg, 63% yield) as yellow oil. ¹H NMR (400 MHz, DMSO-d6) δ 10.02 (s, 1H), 7.48 (d, 1H), 7.38 (d, 1H), 5.46 (s, 2H), 3.79-3.73 (m, 2H), 0.92-0.88 (m, 2H), −0.04 (s, 9H).

### Step C: [5-Chloro-2-iodo-3-(2-trimethylsilylethoxymethoxy) phenyl] methanol

Sodium borohydride (132.0 mg, 3.49 mmol, 1.2 eq) was added in small portions to a solution of 5-chloro-2-iodo-3-(2-trimethylsilylethoxymethoxy)benzaldehyde (1200.0 mg, 2.91 mmol, 1.0 eq) in methanol (12 mL) and water (1.2 mL). Mixture was stirred at 25° C. for 3 hours and then diluted with water (20 mL). The mixture was poured into water (15 ml) and extracted with ethyl acetate (30 mL×3). The combined organic layers were washed with brine (20 mL), dried over anhydrous sodium sulfate, filtered, and the filtrate was concentrated under reduced pressure to give [5-chloro-2-iodo-3-(2-trimethylsilylethoxymethoxy)phenyl]methanol (870 mg, 72% yield) as yellow oil. ¹H NMR (400 MHz, DMSO-d₆) δ 7.13 (d, 1H), 7.09 (d, 1H), 5.59 (t, 1H), 5.36 (s, 2H), 4.40 (d, 2H), 4.03 (q, 1H), 3.74 (t, 2H), 1.99 (s, 2H), 1.17 (t, 2H), 0.88 (t, 2H), −0.04 (s, 9H).

### Step D: 2-[[5-Chloro-2-iodo-3-(methoxymethyl)phenoxy]methoxy]ethyl-trimethyl-silane

To a mixture of [5-chloro-2-iodo-3-(2-trimethylsilylethoxymethoxy)phenyl]methanol (870.0 mg, 2.1 mmol, 1.0 eq) and NaH (60.41 mg, 2.52 mmol, 1.2 eq) in THF (8 mL) was added Mel (0.13 mL, 2.1 mmol, 1.0 eq) in one portion under N₂ at 25° C., and stirred for 5 h. The mixture was poured into water (30 mL) and extracted with ethyl acetate (30 mL×3). The combined organic layers were washed with brine (30 mL), dried over anhydrous sodium sulfate, filtered, and the filtrate was concentrated under reduced pressure. The residue was purified by column chromatography (SiO₂, Petroleum ether/Ethyl acetate=0/1 to 5/1) concentrated under reduced pressure to afford the title compound (800 mg, 89% yield) as colorless oil. ¹H NMR (400 MHz, CD₃OD) δ 7.11 (d, 1H), 7.07 (d, 1H), 5.32 (s, 2H), 4.86 (s, 7H), 4.44 (s, 2H), 3.81 (t, 2H), 3.47 (s, 3H), 0.94 (t, J=8.0 Hz, 2H), 0.01 (s, 9H).

### Step E: 2-[[5-Chloro-3-(methoxymethyl)-2-(4,4,5,5-tetramethyl-1,3,2-dioxaborolan-2-yl)phenoxy]methoxy]ethyl-trimethyl-silane

To a solution of 2-[[5-chloro-2-iodo-3-(methoxymethyl) phenoxy] methoxy] ethyl-trimethyl-silane (800.0 mg, 1.87 mmol, 1.0 eq) and 2-isopropoxy-4,4,5,5-tetramethyl-1,3,2-dioxaborolane (624.87 mg, 3.36 mmol, 1.8 eq) in THF (5 mL) and was added nBuLi (1.12 mL, 2.8 mmol, 1.5 eq) dropwise over 10 min under N₂, stirred at −60° C. for 30 mins. The mixture was poured into NH4Cl solution (15 mL) and extracted with ethyl acetate (20 mL×3). The combined organic layers were washed with brine (20 mL), dried over anhydrous sodium sulfate, filtered, and the filtrate was concentrated under reduced pressure. The residue was purified by preparative TLC (PE/EA 10:1) to give the title compound (260 mg, 33% yield) as colorless oil. ¹H NMR (400 MHz, CD₃OD) δ 7.02 (d, 1H), 6.93 (s, 1H), 5.22 (s, 2H), 4.43 (s, 2H), 3.84-3.74 (m, 2H), 3.30 (s, 3H), 1.37 (s, 12H), 0.98-0.92 (m, 2H), 0.00 (s, 9H).

### Step F: 5-Chloro-3-(methoxymethyl)-2-(4,4,5,5-tetramethyl-1,3,2-dioxaborolan-2-yl)phenol

A solution of 2-[[5-chloro-3-(methoxymethyl)-2-(4,4,5,5-tetramethyl-1,3,2-dioxaborolan-2-yl)phenoxy] methoxy] ethyl-trimethyl-silane (65.0 mg, 0.15 mmol, 1.0 eq) and TFA (0.5 mL) in DCM (0.25 mL) was stirred at 25° C. for 12 h The mixture was poured into water (10 ml) and extracted with EA (10 mL×3), The combined organic layers were washed with brine (20 mL), dried anhydrous sodium sulfate, filtered, and the filtrate was concentrated under reduced pressure. The residue was purified by column chromatography (SiO₂, Petroleum ether/Ethyl acetate=0/1 to I/O) to afford the title compound (45.0 mg, 99% yield) as colorless oil. ¹H NMR (400 MHz, CD₃OD) δ 6.71 (d, 1H), 6.67 (d, 1H), 4.38 (s, 2H), 3.38 (s, 3H), 1.20 (s, 12H).

### Step G: 5-Chloro-2-[2-[[(3R)-1-ethyl-3-piperidyl]amino]oxazolo[4,5-b]pyridin-5-yl]-3-(methoxymethyl)phenol;2,2,2-trifluoroacetic acid salt

A mixture of K₂CO₃ (23.14 mg, 0.17 mmol, 2.5 eq), 5-chloro-3-(methoxymethyl)-2-(4,4,5,5-tetramethyl-1,3,2-dioxaborolan-2-yl)phenol (20.0 mg, 0.07 mmol, 1.0 eq) and 5-bromo-N-[(3R)-1-ethyl-3-piperidyl]oxazolo[4,5-b]pyridin-2-amine; 2,2,2-trifluoroacetic acid (38.3 mg, 0.09 mmol, 1.3 eq) in 1,4-dioxane (1 mL) and water (0.200 mL) was degassed and purged with N₂ three times and XphosPdG3 (5.63 mg, 0.01 mmol, 0.1 eq) was added to the mixture. The mixture was heated under microwave irradiation at 100° C. for 2 h. The mixture was concentrated under reduced pressure and purified by reversed-phase flash (0.1% TFA) and prep-HPLC (Method Column 3_Phenomenex Luna C18 75×30 mm×3 μm Condition water(TFA)-CAN; Begin B 16; End B 46; Gradient Time(min) 8; 100% B; Hold Time(min) 2; FlowRate (mL/min) 25; Injections 1 HPLC 99), after lyophilization the title compound (TFA salt) (10.95 mg, 310% yield) was obtained as a white solid. LCMS: m/z 417.1 [M+H]⁺, ESI pos.

### Example 25

### 4-[2-[[(3R)-1-Ethyl-3-piperidyl]amino]oxazolo[4,5-b]pyridin-5-yl]-3-hydroxy-5-methyl-benzonitrile

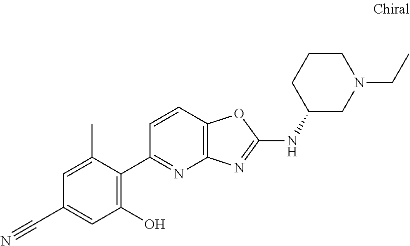 [SMILES: CCN1CCC[C@@H](Nc2nc3nc(-c4c(C)cc(C#N)cc4O)ccc3o2)C1]

### Step A: 4-Amino-3-methoxy-5-methyl-benzonitrile

Two Batches were carried out in parallel. To a solution of 4-bromo-2-methoxy-6-methyl-aniline (25.0 g, 115 mmol, 1.0 eq) in DMF (250 mL) was added Zn(CN)₂ (13.5 g, 115 mmol, 7.34 mL, 1.00 eq) and Pd(PPh₃)₄ (66.8 g, 57.8 mmol, 0.50 eq). The reaction mixture was stirred at 100° C. for 12 hrs. The reaction mixture was poured into H₂O (1.50 L) and extracted with ethyl acetate (1.00 L×3). The organic phase was washed with brine (1.00 L×3), dried over Na₂SO₄, filtered and concentrated under reduced pressure to give a residue, which was purified by column chromatography (SiO₂, petroleum ether/ethyl acetate=100/1 to 0/1) to yield the title compound (28.0 g, 75% yield) as a yellow solid.

### Step B: 4-Bromo-3-methoxy-5-methyl-benzonitrile

To a solution of CuBr (46.4 g, 323 mmol, 9.86 mL, 1.50 eq) in MeCN (180 mL) was added t-BuONO (33.3 g, 323 mmol, 38.5 mL, 1.50 eq) and stirred at 65° C. Then solution of 4-amino-3-methoxy-5-methyl-benzonitrile (35.0 g, 215 mmol, 1.00 eq) in MeCN (180 mL) at 65° C. was added. The mixture was stirred at 65° C. for 3.5 hrs. After cooling to rt, sat. aq. Na₂SO₃ (400 mL) and sat. aq. NH₄Cl (200 mL) was added to the mixture and extracted with ethyl acetate (500 mL×3). The organic phase was washed with brine (500 mL×2), dried over Na₂SO₄, filtered and concentrated under reduced pressure to give a residue. The residue was purified by column chromatography (SiO₂, petroleum ether/ethyl acetate=100/1 to 0/1, Rf=0.75) to give the title compound (20.7 g, 42% yield) as a white solid.

### Step C: 3-Methoxy-5-methyl-4-(4,4,5,5-tetramethyl-1,3,2-dioxaborolan-2-yl)benzonitrile

To a solution of 4-bromo-3-methoxy-5-methyl-benzonitrile (18.0 g, 79.6 mmol, 1.00 eq) in DMF (180 mL) was added B₂Pin₂ (30.3 g, 119 mmol, 1.50 eq) and AcOK (35.1 g, 358 mmol, 4.50 eq). The mixture was stirred at 20° C. for 0.5 hr and Pd(dppf)Cl₂·CH₂Cl₂ (13.0 g, 15.9 mmol, 0.20 eq) was added. The mixture was stirred at 100° C. for 12 hrs. After cooling to rt, the mixture was filtered with diatomite and diluted with H₂O (500 mL) and extracted with ethyl acetate (800 mL×3). The organic phase was washed with brine (800 mL×3), dried over Na₂SO₄, filtered and concentrated under reduced pressure. The residue was purified by column chromatography (SiO₂, petroleum ether/ethyl acetate=100/1 to 1/1, Rf=0.30) to give the title compound (18.0 g, 83% yield) as a white solid.

### Step D: (4-Cyano-2-hydroxy-6-methyl-phenyl)boronic acid

A solution of 3-methoxy-5-methyl-4-(4,4,5,5-tetramethyl-1,3,2-dioxaborolan-2-yl)benzonitrile (17.0 g, 96.0 mmol, 1.00 eq) in DCM (170 mL) was cooled to 0° C. and BBr₃ (38.9 g, 155 mmol, 2.50 eq) was added dropwise at 0° C. The mixture was stirred at 0° C. for 0.5 hr. The mixture was poured into H₂O (200 mL), filtered, and the cake was collected and triturated with ethyl acetate (20 mL) to give the title compound (4.67 g, 42% yield) as a gray solid. LCMS: m/z 178.1 [M+H]⁺, ESI pos.

### Step E: 4-[2-[[(3R)-1-Ethyl-3-piperidyl]amino]oxazolo[4,5-b]pyridin-5-yl]-3-hydroxy-5-methyl-benzonitrile

Aforementioned (5-chlorooxazolo[4,5-b]pyridin-2-yl)-[(3R)-1-ethyl-3-piperidyl]amine (241 mg, 858.4 umol, 1.0 eq), aforementioned (4-cyano-2-hydroxy-6-methyl-phenyl)boronic acid (Example 25, step D) (280.71 mg, 1.46 mmol, 1.7 eq) and cesium carbonate (839.07 mg, 2.58 mmol, 3.0 eq) were dissolved in 1,4-dioxane (5.9 mL) and water (1.48 mL). Then argon was bubbled through the mixture for 2 min followed by addition of the catalyst methanesulfonato(2-dicyclohexylphosphino-2′,4′,6′-tri-1-propyl-1,1′-biphenyl)(2′-amino-1,1′-biphenyl-2-yl)palladium(ii) (XPhos Pd G3) (108.99 mg, 128.76 umol, 0.15 eq). The sealed tube was stirred at 100° C. for 16 hours. After cooling to rt, the reaction mixture was extracted with ethyl acetate (2×80 mL) and half-saturated aq. Ammonium chloride solution (80 mL). The organic layers were washed with water (80 mL) and brine (80 mL). The combined organic layers were dried over sodium sulfate, filtered and concentrated in vacuo. The crude was purified by flash chromatography (SiO₂; 0-50% ethyl acetate in heptane; then ethyl acetate:methanol 9:1 (v/v) followed by crystallisation with ethyl acetate/heptane to afford the title compound (135 mg, 41% yield) as light yellow solid. LC-MS: 378.2 [M+H]⁺, ESI pos.

### Example 26

### 3-Hydroxy-5-methyl-4-[2-[[(3R)-1-methyl-3-piperidyl]amino]oxazolo[4,5-b]pyridin-5-yl]benzonitrile

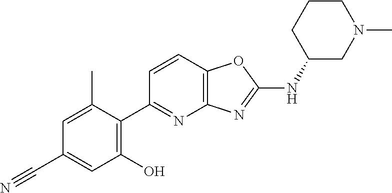 [SMILES: Cc1cc(C#N)cc(O)c1-c1ccc2oc(N[C@@H]3CCCN(C)C3)nc2n1]

### Step 1: 3-Hydroxy-5-methyl-4-[2-[[(3R)-1-methyl-3-piperidyl]amino]oxazolo[4,5-b]pyridin-5-yl]benzonitrile

Aforementioned 5-chloro-N-[(3R)-1-methyl-3-piperidyl]oxazolo[4,5-b]pyridin-2-amine (Example 15, step 1) (112 mg, 0.420 mmol, 1.0 eq), (4-cyano-2-hydroxy-6-methyl-phenyl)boronic acid (Example 25, step D) (137.3 mg, 0.71 mmol, 1.7 eq) and potassium carbonate (261.2 mg, 1.9 mmol, 4.5 eq) were dissolved in 1,4-dioxane (3 mL) and water (1.5 mL). Then argon was bubbled through the mixture for 2 min followed by the addition of the catalyst 1,1′-bis(diphenylphosphino)ferrocene-palladium(ii)-dichloride dichloromethane complex (51.4 mg, 0.063 mmol, 0.15 eq). The sealed tube was stirred at 90° C. for 16 h. Afterwards, the reaction mixture was extracted with ethyl acetate (2×30 mL) and half-saturated aq. Ammonium chloride solution (30 mL). The organic layers were washed with water (30 mL) and brine (30 mL). The combined organic layers were dried over sodium sulfate, filtered and concentrated in vacuo. The crude was purified by flash chromatography (SiO₂; 0-50% ethyl acetate in heptane; then ethyl acetate:methanol 9:1 (v/v) followed by crystallisation with ethyl acetate/heptane to afford the title compound (51 mg, 32% yield) as an off-white solid. LC-MS: 364.2 [M+H]⁺, ESI pos.

### Example 27, 27a and 27b

(rac)-3-Hydroxy-5-methyl-4-[2-(6-methyl-3,3a,4,5,7,7a-hexahydro-2H-pyrrolo[2,3-c]pyridin-1-yl)oxazolo[4,5-b]pyridin-5-yl]benzonitrile and respective enantiomers 27A and 27B

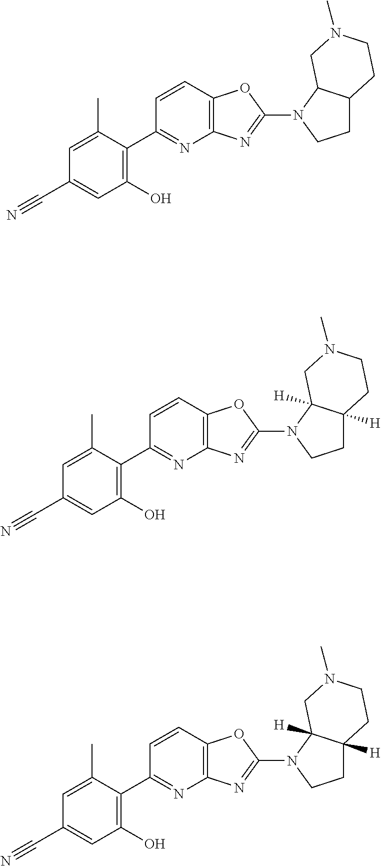 [SMILES: Cc1cc(C#N)cc(O)c1-c1ccc2oc(N3CCC4CCN(C)CC43)nc2n1 ; Cc1cc(C#N)cc(O)c1-c1ccc2oc(N3CC[C@@H]4CCN(C)C[C@@H]43)nc2n1 ; Cc1cc(C#N)cc(O)c1-c1ccc2oc(N3CC[C@H]4CCN(C)C[C@H]43)nc2n1]

### Step 1: 5-Chloro-2-(6-methyl-3,3a,4,5,7,7a-hexahydro-2H-pyrrolo[2,3-c]pyridin-1-yl)oxazolo[4,5-b]pyridine

To a mixture of 5-chloro-2-(methylthio)oxazolo[4,5-b]pyridine (CAS #1783370-92-2, 260 mg, 1.23 mmol, 1.00 eq) and 6-methyl-1,2,3,3a,4,5,7,7a-octahydropyrrolo[2,3-c]pyridine (CAS #1443980-22-0, 224 mg, 1.60 mmol, 1.30 eq) in 1,4-dioxane (4.0 mL) and N-methyl-2-pyrrolidinone (2.0 mL) was added triethylamine (374 mg, 0.515 mL, 3.69 mmol, 3.00 eq). The reaction mixture was heated at 120° C. and stirred for 2 hours and at 150° C. for 20 hours. The reaction mixture was cooled to room temperature and extracted with a mixture of ethyl acetate:MTBE (1:1) and water. The aqueous layer was backextracted with a mixture of ethyl acetate:MTBE (1:1). The organic layers were washed with water and brine. The combined organic layers were dried over sodium sulfate, filtered and concentrated in vacuo. The crude product was absorbed on ISOLUTE HM-N and purified by flash chromatography (silica gel, 12 g, gradient 0% to 10% methanol in dichlormethane) to afford the title compound (313 mg, 86% yield) as a yellow solid. LC-MS: m/z 293.1 [M+H]⁺, ESI pos.

### Step 2: (rac)-3-Hydroxy-5-methyl-4-[2-(6-methyl-3,3a,4,5,7,7a-hexahydro-2H-pyrrolo[2,3-c]pyridin-1-yl)oxazolo[4,5-b]pyridin-5-yl]benzonitrile

To a mixture of 5-chloro-2-(6-methyl-3,3a,4,5,7,7a-hexahydro-2H-pyrrolo[2,3-c]pyridin-1-yl)oxazolo[4,5-b]pyridine (Example 20, step 1) (160 mg, 0.54 mmol, 1.00 eq) and (4-cyano-2-hydroxy-6-methyl-phenyl)boronic acid (Example 25, step D) (208 mg, 1.08 mmol, 2.00 eq) in 1,4-dioxane (6.0 mL) and water (3.0 mL) was added under stirring potassium carbonate (359 mg, 2.60 mmol, 4.80 eq) at room temperature. Argon was bubbled through the reaction mixture for three minutes. Then, 1,1′-bis(diphenylphosphino)ferrocene-palladium(ii)dichloride dichloromethane complex (88 mg, 0.11 mmol, 0.20 eq) was added under stirring at room temperature. The tube was sealed and stirred at 100° C. for 20 hours. The reaction mixture was extracted with ethyl acetate and half-saturated aq. NH₄Cl-solution. The organic layers were washed with water and brine. The combined organic layers were dried over sodium sulfate, filtered and concentrated in vacuo. The crude product was adsorbed on ISOLUTE HM-N and purified by flash chromatography (silica gel, 40 g, gradient 0% to 100% (dichloromethane:methanol:NH₄OH 110:10:1) in dichloromethane) to afford the title compound (100 mg, 43% yield, 90% purity) as dark brown foam. LC-MS: m/z 390.2 [M+H]⁺, ESI pos.

### Step 3: 4-[2-[(3aS,7aR or 3aR,7aS)-6-methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile and 4-[2-[(3aR,7aS or 3aS,7aR)-6-methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile

Racemate (rac)-3-hydroxy-5-methyl-4-[2-(6-methyl-3,3a,4,5,7,7a-hexahydro-2H-pyrrolo[2,3-c]pyridin-1-yl)oxazolo[4,5-b]pyridin-5-yl]benzonitrile (Example 20, step 2) was separated by chiral SFC (column: Chiralpak IJ, eluent B: 20% methanol+0.2% diethylamine) to afford the two enantiomers example 27A (first eluting, Rt=1.31 min) (35 mg, 38% yield) as a light brown foam; LC-MS: m/z 390.2 [M+H]⁺, ESI pos and example 27B (second eluting, Rt=1.94 min) (28 mg, 30% yield) as a light brown foam. LC-MS: m/z 390.2 [M+H]⁺, ESI pos.

### Example 28

### 4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid salt

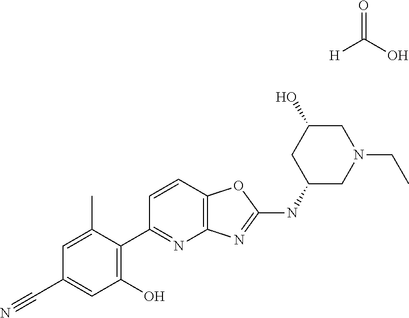 [SMILES: O=CO ; CCN1C[C@@H](O)C[C@@H](Nc2nc3nc(-c4c(C)cc(C#N)cc4O)ccc3o2)C1]

### Step 1: (3R,5S)-3-[(5-Chlorooxazolo[4,5-b]pyridin-2-yl)aminol-5-hydroxy-piperidine-1-carboxylic acid tert-butyl ester

To aforementioned 5-chloro-2-(methylthio)oxazolo[4,5-b]pyridine (CAS #1783370-92-2, 491 mg, 2.32 mmol, 1.0 eq) in 1,4-dioxane (3 mL) and N-methyl-2-pyrrolidinone (6 mL) was added (3R,5S)-3-amino-5-hydroxy-piperidine-1-carboxylic acid tert-butyl ester (653.6 mg, 3.02 mmol, 1.3 eq) followed by triethylamine (423.4 mg, 583.2 μL, 4.18 mmol, 1.8 eq). The light brown solution was stirred in a sealed tube at 150° C. for 16 hours. Afterwards, the reaction was quenched with water (5 mL) and extracted with ethylacetate (2×80 mL). Organic layers were washed with brine (80 mL), dried over sodium sulfate, filtered and concentrated in vacuo. The residue was purified by flash chromatography (ISCO, 25 g SiO₂; 0-50% ethyl acetate in heptane; then ethyl acetate:methanol 9:1 (v/v) to afford (3R,5S)-3-[(5-chlorooxazolo[4,5-b]pyridin-2-yl)amino]-5-hydroxy-piperidine-1-carboxylic acid tert-butyl ester (441 mg, 49% yield) as light yellow foam. LC-MS: m/z 369.2 [M+H]⁺, ESI pos.

### Step 2: (3S,5R)-5-[(5-Chlorooxazolo[4,5-b]pyridin-2-yl)amino]piperidin-3-ol;hydrogen chloride salt

To a solution of (3R,5S)-3-[(5-chlorooxazolo[4,5-b]pyridin-2-yl)amino]-5-hydroxy-piperidine-1-carboxylic acid tert-butyl ester (441 mg, 1.2 mmol, 1.0 eq) in DCM (10 mL) and methanol (5 mL) was added at rt 4 M HCl in 1,4-dioxane (3.59 g, 3.0 mL, 12 mmol, 10 eq) dropwise. The reaction mixture was stirred at 23° C. for 16 h. The reaction mixture was concentrated in vacuo to give the crude (3S,5R)-5-[(5-chlorooxazolo[4,5-b]pyridin-2-yl)amino]piperidin-3-ol;hydrogen chloride salt (415 mg, 102% yield) as light yellow foam which was used without further purification in the next step. LC-MS: m/z 269.1 [M+H]⁺, ESI pos.

### Step 3: (3S,5R)-5-[(5-Chlorooxazolo[4,5-b]pyridin-2-yl)amino]-1-ethyl-piperidin-3-ol

To a suspension of (3S,5R)-5-[(5-chlorooxazolo[4,5-b]pyridin-2-yl)amino]piperidin-3-ol;hydrochloride (414 mg, 1.22 mmol, 1.0 eq) in dry DCM (16.9 mL) was added acetaldehyde (134.5 mg, 172 uL, 3.05 mmol, 2.5 eq) followed by sodium acetate (250 mg, 3.05 mmol, 2.5 eq) under ice-bath cooling. Sodium triacetoxyborohydride (466 mg, 2.2 mmol, 1.8 eq) was added at 0° C. The reaction mixture was stirred at 0° C. for 5 min and at rt for 3 h. Then, the reaction mixture was carefully basified with aq. NaHCO₃ solution (25 mL) and then extracted with dichloromethane (3×60 mL). The combined organic layers were dried over sodium sulfate, filtered and concentrated in vacuo. The crude product was adsorbed on ISOLUTE HM-N and purified by flash chromatography (silica gel, 24 g, 0% to 10% methanol in dichloromethane) to afford the title compound (187 mg, 52% yield) as light brown solid. LC-MS: m/z 297.1 [M+H]⁺, ESI pos.

### Step 4: 4-[2-[[(3R,5S)-1-Ethyl-5-hydroxy-3-piperidyl]amino]oxazolo[4,5-b]pyridin-5-yl]-3-hydroxy-5-methyl-benzonitrile;formic acid salt

A mixture of (3S,5R)-5-[(5-chlorooxazolo[4,5-b]pyridin-2-yl)amino]-1-ethyl-piperidin-3-ol (Example 28, step 3) (55 mg, 166.8 umol, 1.0 eq), (4-cyano-2-hydroxy-6-methyl-phenyl)boronic acid (Example 25, step D) (64.2 mg, 333.6 umol, 2.0 eq), potassium carbonate (103.7 mg, 750.6 umol, 4.5 eq) and 1,1′-bis(diphenylphosphino)ferrocene-palladium(ii)dichloride dichloromethane complex (20.43 mg, 25.0 umol, 0.15 eq) in 1,4-dioxane (2 mL) and water (1 mL) was flushed with argon and stirred at 100° C. for 16 h. The reaction mixture was extracted with ethyl acetate (2×20 mL) and half-saturated aq. ammonium chloride solution (20 mL). The organic layers were washed with water (30 mL) and brine (30 mL). The combined organic layers were dried over sodium sulfate, filtered and concentrated in vacuo. The crude product was adsorbed on ISOLUTE HM-N and purified by flash chromatography (silica gel, 12 g, gradient 0% to 100% (DCM:methanol:NH₄OH 110:10:1) in DCM) to afford followed by preparative HPLC (column: YMC-Triart C₁₈, 12 nm, 5 μm, 100×30 mm, gradient 5 to 50% MeCN in (Water+0.1% HCOOH)) to afford the title compound (12 mg, 16% yield) as white amorphous freeze-dried solid. LC-MS: m/z 392.2 [M−H]⁻, ESI neg.

### Example 29

### (rac)-4-[2-(1,2,3,5,6,7,8,8a-Octahydroindolizin-8-ylamino)oxazolo[4,5-b]pyridin-5-yl]-3-hydroxy-5-methyl-benzonitrile

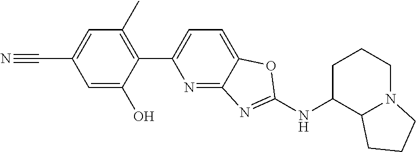 [SMILES: Cc1cc(C#N)cc(O)c1-c1ccc2oc(NC3CCCN4CCCC34)nc2n1]

### Step 1: (rac)-(5-Chlorooxazolo[4,5-b]pyridin-2-yl)-indolizidin-8-yl-amine

To a suspension of 5-chloro-2-(methylthio)oxazolo[4,5-b]pyridine (CAS #1783370-92-2, 200 mg, 947 umol, 1.0 eq) and rac-(8S,8aS)-octahydro-8-indolizinamine-dihydrochloride (262.4 mg, 1.23 mmol, 1.3 eq) in 1,4-dioxane (1.5 mL) and N-methyl-2-pyrrolidinone (1.5 mL) was added dropwise triethylamine (383 mg, 528 uL, 3.8 mmol, 4.0 eq) at rt. The brown suspension was sealed, and heated at 120° C. for 16 h. The dark-orange-brown reaction mixture was cooled to rt and extracted twice with ethyl acetate:tBME (v/v) 1:1 (2×50 mL). The organic layers were washed with water (20 mL) and brine (20 mL). The aqueous layers were back extracted with ethyl acetate:tBME (v/v) 1:1 (50 mL). The combined organic phases were dried over sodium sulfate, filtered and concentrated in vacuo. The crude product was absorbed on ISOLUTE HM-N and purified by flash chromatography (SiO₂; gradient 0%-10% methanol in DCM) to afford the title compound (64 mg, 21%) as light brown solid. LC-MS: m/z 293.1 M+H]⁺, ESI pos.

### Step 2: (rac)-4-[2-(1,2,3,5,6,7,8,8a-Octahydroindolizin-8-ylamino)oxazolo[4,5-b]pyridin-5-yl]-3-hydroxy-5-methyl-benzonitrile

(rac)-(5-Chlorooxazolo[4,5-b]pyridin-2-yl)-indolizidin-8-yl-amine (64 mg, 0.2 mmol, 1.0 eq) and (4-cyano-2-hydroxy-6-methyl-phenyl)boronic acid (Example 25, step D) (69.6 mg, 394 umol, 2.0 eq) were dissolved in 1,4-dioxane (2 mL) and water (1 mL), and potassium carbonate (122.4 mg, 885.4 umol, 4.5 eq) was added. Afterwards, the mixture was flushed with argon for 3 minutes, followed by the addition of 1,1′-bis(diphenylphosphino)ferrocene-palladium(ii)dichloride dichloromethane complex (24.1 mg, 29.51 umol, 0.15 eq). The dark red reaction suspension was sealed and stirred at 100° C. for 16 h. Then, the reaction mixture was cooled to rt and extracted with DCM (2×20 mL) and saturated aq. Ammonium chloride solution (20 mL). The organic layers were washed with water (10 mL) and brine (10 mL). The aqueous layers were back extracted with DCM (20 mL). The combined organic phases were dried over sodium sulfate, filtered and concentrated in vacuo. The crude product was adsorbed on ISOLUTE H-N-M and purified by flash chromatography (SiO2; 0%-60% DCM:methanol:NH₄OH (v/v) 110:10:1 in DCM) followed by further purification by crystallization (dissolved in heptane:ethyl acetate (v/v) 1:1; powdered and evaporated) to afford the title compound (27 mg, 35%) as pink solid. LC-MS: m/z 390.3 [M+H]⁺, ESI pos.

### Example 30

### 3-(Difluoromethyl)-2-[2-[[(3R)-1-methyl-3-piperidyl]amino]oxazolo[4,5-b]pyridin-5-yl]-5-(trifluoromethyl)phenol;2,2,2-trifluoroacetic acid salt

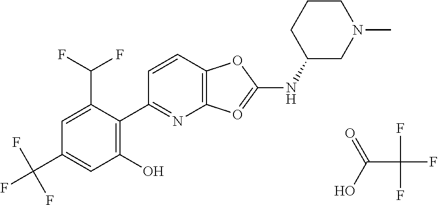

### Step A: 3-Hydroxy-N-methoxy-N-methyl-5-(trifluoromethyl)benzamide

To a mixture of 3-hydroxy-5-(trifluromethyl)benzoic acid (3.00 g, 14.55 mmol, 1.0 eq) and O,N-dimethylhydroxylamine HCl (1703.59 mg, 17.47 mmol, 1.2 eq) in DMF (30 mL) was added DIEA (5880.07 mg, 58.22 mmol, 4.0 eq), EDCI (4169.9 mg, 21.83 mmol, 1.5 eq) and HOBt (1571.9 mg, 11.64 mmol, 0.8 eq), and the mixture was stirred at 25° C. for 2 hours. The reaction mixture was quenched by 30 mL water, then extracted with EtOAc (40 mL×3), washed with brine (30 mL×2), dry over anhydrous Na₂SO₄, filtrated an the filtrated was concentrated under reduce pressure. The residue was purified by column (PE:EtOAc=10:1 to 3:1) to afford the title compound (2500 mg, 69% yield) as a white solid. LC-MS: m/z 250.0 [M+H]⁺, ESI pos.

### Step B: 3-Hydroxy-2-iodo-N-methoxy-N-methyl-5-(trifluoromethyl)benzamide

To a mixture of 3-hydroxy-N-methoxy-N-methyl-5-(trifluoromethyl)benzamide (6.4 g, 25.7 mmol, 1.0 eq) in toluene (65 mL) was added NaH (2.05 g, 51.37 mmol, 2.0 eq) at 0° C., and the mixture was stirred at 0° C. for 10 minibus, then I₂ (5.2 g, 20.55 mmol, 0.8 eq) was added to the mixture and the mixture was stirred at 25° C. for 12 hours. The reaction mixture was quenched by 20 mL water, then extracted by EA (30 mL×3), washed with brine (40 mL×2), dry over anhydrous Na₂SO₄, filtrated and the filtrate was concentrated under reduce pressure to get a yellow solid. The solid was purified by column (PE:EA=20:1 to 10:1) to afford the title compound (3200 mg, 33% yield) as a yellow solid. ¹H NMR (400 MHz, CD₃OD) δ 7.07 (s, 1H), 7.05 (s, 1H), 3.52 (s, 3H), 3.38 (s, 3H).

### Step C: 3-(Benzyloxy)-2-iodo-N-methoxy-N-methyl-5-(trifluoromethyl)benzamide

To a solution of 3-hydroxy-2-iodo-N-methoxy-N-methyl-5-(trifluoromethyl)benzamide (3.20 g, 8.53 mmol, 1.0 eq) in DMF (35 mL) was added K₂CO₃ (2354.7 mg, 17.06 mmol, 2.0 eq) and BnBr (2.18 g, 12.8 mmol, 1.5 eq), then the mixture was stirred at 25° C. for 2 hours. The reaction mixture was quenched by 10 mL, extracted by EA (20 mL×3), washed with brine (20 mL×2), dry over anhydrous Na₂SO₄, filtrated and the filtrate was concentrated under reduce pressure to get a yellow solid. The solid was purified by column (PE:EA=20:1 to 10:1) to afford 3-(benzyloxy)-2-iodo-N-methoxy-N-methyl-5-(trifluoromethyl)benzamide (3.50 g, 88% yield) as a white solid. ¹H NMR (400 MHz, CD₃OD) a 7.53 (d, 3H), 7.41-7.38 (m, 3H), 7.35-7.31 (m, 2H), 7.27 (s, 1H), 7.23 (s, 1H), 5.28 (s, 2H), 3.91 (s, 1H), 3.51 (s, 3H), 3.39 (s, 3H), 3.12 (s, 1H).

### Step D: (3-(Benzyloxy)-2-iodo-5-(trifluoromethyl)phenyl)methanol

To a mixture of 3-(benzyloxy)-2-iodo-N-methoxy-N-methyl-5-(trifluoromethyl)benzamide (3.50 g, 7.52 mmol, 1.0 eq) in THF (40 mL) was added DIBAL-H (22.57 mL, 22.57 mmol, 3.0 eq) dropwise at −60° C., and the mixture was stirred at −60° C. for 3 hours. The reaction mixture was dropwise added to 20 mL saturate NH₄Cl solution, then extracted by EA (20 mL×3), washed with brine (20 mL×2), filtrated and the filtrate was concentrated under reduce pressure to get a yellow solid. The solid was purified by column (PE:EA=20:1 to 10:1) to afford (3-(benzyloxy)-2-iodo-5-(trifluoromethyl)phenyl)methanol (1800 mg, 59% yield) as a white solid. ¹H NMR (400 MHz, CD₃OD) a 7.45-7.40 (m, 3H), 7.31-7.27 (m, 2H), 7.24-7.20 (m, 1H), 7.10 (d, 1H), 5.16 (s, 2H).

### Step E: 3-(Benzyloxy)-2-iodo-5-(trifluoromethyl)benzaldehyde

To a mixture of 3-(benzyloxy)-2-iodo-5-(trifluoromethyl)benzaldehyde (1800.0 mg, 4.41 mmol, 1.0 eq) in DCM (20 mL) was added MnO₂ (5524.15 mg, 22.05 mmol, 5.0 eq), then the mixture stirred at 25° C. for 1 hour. The reaction mixture was filtrated and the filtrate was concentrated under reduce pressure to afford 3-(benzyloxy)-2-iodo-5-(trifluoromethyl)benzaldehyde (1500 mg, 84% yield) as a white solid. ¹H NMR (400 MHz, CD₃OD) a 10.21 (m, 1H), 7.56-7.52 (m, 3H), 7.41-7.38 (m, 2H), 7.34-7.31 (m, 1H), 7.20 (d, 1H), 5.26 (s, 2H).

### Step F: 1-(Benzyloxy)-3-(difluoromethyl)-2-iodo-5-(trifluoromethyl)benzene

To a mixture of 3-(benzyloxy)-2-iodo-5-(trifluoromethyl)benzaldehyde (1500.0 mg, 3.69 mmol, 1.0 eq) in DCM (30 mL) was added diethylaminosulfur trifluoride (893.04 mg, 5.54 mmol, 1.5 eq) at 0° C., then the mixture was stirred at 25° C. for 12 hours. The reaction mixture was quenched by 20 mL ice-water, then extracted by EtOAc (30 mL×3), washed with brine (20 mL×2), dry over anhydrous Na₂SO₄, filtrated and the filtrate was concentrated under reduce pressure to get a white solid. The solid was purified by column (PE:EA=20:1 to 10:1) to afford 1-(benzyloxy)-3-(difluoromethyl)-2-iodo-5-(trifluoromethyl)benzene (1300 mg, 82% yield) as a white solid. ¹H NMR (400 MHz, CD₃OD) δ 7.54 (d, 2H), 7.44-7.38 (m, 4H), 7.36-7.32 (m, 1H), 7.00 (t, 1H), 5.31 (s, 2H)

### Step G: 2-(2-(Benzyloxy)-6-(difluoromethyl)-4-(trifluoromethyl)phenyl)-4,4,5,5-tetramethyl-1,3,2-dioxaborolane

To a mixture of 1-(benzyloxy)-3-(difluoromethyl)-2-iodo-5-(trifluoromethyl)benzene (400.0 mg, 0.93 mmol, 1.0 eq) and nBuLi (0.56 mL, 1.4 mmol, 1.5 eq) in THF (5 mL) and was dropwise added 2-isopropoxy-4,4,5,5-tetramethyl-1,3,2-dioxaborolane (312.9 mg, 1.68 mmol, 1.8 eq) under N₂, and the mixture stirred at −60° C. for 30 minutes. The mixture was poured into NH₄Cl solution (15 mL) and extracted with ethyl acetate (20 mL×3). The combined organic layers were washed with brine (20 mL), dried over anhydrous Na₂SO₄, filtered and the filtrate was concentrated under reduced pressure. The residue was purified by column (PE:EA=20:1 to 10:1) to give 2-(2-(benzyloxy)-6-(difluoromethyl)-4-(trifluoromethyl)phenyl)-4,4,5,5-tetramethyl-1,3,2-dioxaborolane (50.0 mg, 13% yield) as whites solid. ¹H NMR (400 MHz, CD₃OD) δ 7.51 (s, 1H), 7.50 (s, 1H), 7.41-7.32 (m, 5H), 5.16 (s, 2H), 1.26 (s, 12H).

### Step H: 5-Bromo-N-[(3R)-1-methyl-3-piperidyl]oxazolo[4,5-b]pyridin-2-amine

(3R)-1-Methylpiperidin-3-amine dihydrochloride (172.0 mg, 0.92 mmol, 1.12 eq, CAS: 1157849-50-7) and triethylamine (0.4 mL, 2.87 mmol, 3.48 eq) were added to stirred solution of 5-bromo-2-methylsulfanyl-oxazolo[4,5-b]pyridine (202.0 mg, 0.82 mmol, 1.0 eq) in 1,4-Dioxane (4 mL) and the reaction mixture was heated to 90° C. The reaction was stirred for 3 days. Triethylamine (0.3 mL, 2.15 mmol, 2.6 eq) and (3R)-1-ethylpiperidin-3-amine dihydrochloride (164.0 mg, 0.82 mmol, 1.0 eq) was added and the reaction left to stir at 90° C. for 2 h. Then additional (3R)-1-ethylpiperidin-3-amine dihydrochloride (42.0 mg, 0.21 mmol, 0.25 eq) and triethylamine (0.15 mL, 1.08 mmol, 1.3 eq) were added and the reaction was stirred at 90° C. for 18 h. The reaction was allowed to cool to r.t. and concentrated in vacuo. The crude reaction mixture was purified by chromatography on silica gel (40 g, 0-20% MeOH (with 0.7M NH₃)/EtOAc) to afford the title compound (222.0 mg, 84% yield) as a light brown solid. LC-MS: m/z 311.1/313.1 [M+H]⁺, ESI pos.

### Step I: (R)-5-(2-(Benzyloxy)-6-(difluoromethyl)-4-(trifluoromethyl)phenyl)-N-(1-methylpiperidin-3-yl)oxazolo[4,5-b]pyridin-2-amine

To a solution of 2-(2-(benzyloxy)-6-(difluoromethyl)-4-(trifluoromethyl)phenyl)-4,4,5,5-tetramethyl-1,3,2-dioxaborolane (100 mg, 0.23 mmol, 1.0 eq) and 5-bromo-N-[(3R)-1-methyl-3-piperidyl]oxazolo[4,5-b]pyridin-2-amine (Example 30, step H) (87.2 mg, 0.28 mmol, 1.2 eq) in a mixture of 1,4-dioxane (5 mL) and water (1 mL) was added K₂CO₃ (74.26 mg, 0.7 mmol, 3.0 eq) and Pd(dppf)₂Cl₂ (6.74 mg, 0.02 mmol, 0.1 eq), and the mixture was stirred at 90° C. for 2 hours under N₂. The reaction mixture was concentrated under reduce pressure to remove the solvent to get a brown solid. The solid was purified by reversed phase flash (0.1% TFA water/ACN condition). The eluate was dried by lyophilization to get a white solid to the title compound (20.0 mg, 16% yield) as a white solid. LC-MS: m/z 533.2 [M+H]⁺, ESI pos.

### Step I: (R)-3-(Difluoromethyl)-2-(2-((1-methylpiperidin-3-yl)amino)oxazolo[4,5-b]pyridin-5-yl)-5-(trifluoromethyl)phenol

To a mixture of (R)-5-(2-(benzyloxy)-6-(difluoromethyl)-4-(trifluoromethyl)phenyl)-N-(1-methylpiperidin-3-yl)oxazolo[4,5-b]pyridin-2-amine (10.0 mg, 0.02 mmol, 1.0 eq) in EtOAc (1 mL) was added Pd/C (20.0 mg) and the mixture was stirred at 25° C. for 1 hour. The reaction mixture was filtrated and the filtrate was concentrated under reduce pressure to get a white solid. The solid was purified by reversed phase flash (0.1% TFA water/ACN condition). The eluate was dried by lyophilization to get a white solid to afford the title compound (4.47 mg, 42% yield) as a white solid. LC-MS: m/z 443.2 [M+H]⁺, ESI pos.

### Example 31

### 2-[2-[[(3R)-1-Ethyl-3-piperidyl]amino]oxazolo[4,5-b]pyridin-5-yl]-3-methoxy-5-(trifluoromethyl)phenol;2,2,2-trifluoroacetic acid salt

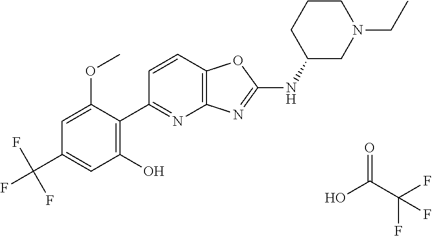 [SMILES: CCN1CCC[C@@H](Nc2nc3nc(-c4c(O)cc(C(F)(F)F)cc4OC)ccc3o2)C1 ; O=C(O)C(F)(F)F]

### Step A: 3-Amino-5-(trifluoromethyl)phenol

A solution of 3-nitro-5-(trifluoromethyl)phenol (8000.0 mg, 38.63 mmol, 1.0 eq) in methanol (80 mL) was evacuated and refilled with nitrogen three times, then Pd/C (200 mg, 10% purity) was added and the mixture was evacuated and refilled with hydrogen three times. The reaction mixture was stirred at 25° C. for 3 hours under hydrogen atmosphere (15 psi). The reaction mixture was filtrated and the solid was washed with MeOH (50 mL×3), the filtrate was concentrated under reduced pressure to afford 3-amino-5-(trifluoromethyl)phenol (6400 mg, 94% yield) as a yellow solid. LC-MS: m/z 178.0 [M+H]⁺, ESI pos.

### Step B: 5-(Trifluoromethyl)benzene-1,3-diol

A solution of 3-amino-5-(trifluoromethyl)phenol (2000.0 mg, 11.29 mmol, 1.0 eq) in concentrated sulfuric acid (8.0 mL, 150.1 mmol, 13.29 eq) and water (7 mL) was cooled to 0° C., then a solution of NaNO₂ (1168.53 mg, 16.94 mmol, 1.5 eq) in water (4 mL) was added dropwise, after stirring for 15 minutes, the excess nitrous acid was destroyed by addition of Urea (339.07 mg, 5.65 mmol, 0.5 eq), then this cool solution was added to a refluxing saturated solution of copper sulfate (25.0 g, 156.64 mmol, 13.87 eq) in water (100 mL) dropwise and stirred at this temperature for 30 minutes. After cooling to ambient temperature, the reaction mixture was extracted with EA (100 mL×3), the combined organic layer was dried over anhydrous sodium sulfate, filtrated and the filtrate was concentrated under reduced pressure. The residue was purified by C18 column chromatograph (0.1% NH₄OH in water/MeCN) and the desired fractions were extracted with EA (100 mL×3), washed with brine (20 mL), the combined organic layer was dried over anhydrous sodium sulfate, filtrated and the filtrate was concentrated under reduced pressure to afford 5-(trifluoromethyl)benzene-1, 3-diol (1.20 g, 60% yield) as red oil. LC-MS: m/z 354.9 [2M−H]⁻, ESI neg.

### Step C: 2-Iodo-5-(trifluoromethyl)benzene-1,3-diol

To a solution of 5-(trifluoromethyl)benzene-1, 3-diol (1200 mg, 6.74 mmol, 1.0 eq) in THF (70 mL) and water (70 mL) was added a solution of iodine (1710.02 mg, 6.74 mmol, 1.0 eq) in THF (10 mL) dropwise at 0° C., followed by sodium bicarbonate (566.01 mg, 6.74 mmol, 1.0 eq). after stirring for 10 minutes at 0° C., the reaction mixture was warmed to 25° C. and stirred at this temperature for another 50 minutes. The reaction mixture was quenched with a saturated aqueous solution of sodium sulfite (100 mL), extracted with EA (100 mL×3), washed with brine (30 mL). The combined organic layer was dried over anhydrous sodium sulfate, filtrated and the filtrate was concentrated under reduced pressure. The residue was purified by silica gel column chromatograph (PE to PE:EA=10:1) to afford 2-iodo-5-(trifluoromethyl)benzene-1, 3-diol (1000 mg, 49% yield) as a yellow solid. LC-MS: m/z 302.8, [M−H]⁻, ESI neg.

### Step D: 3-(Benzyloxy)-2-iodo-5-(trifluoromethyl)phenol

To a solution of 2-iodo-5-(trifluoromethyl)benzene-1, 3-diol (200 mg, 0.66 mmol, 1.0 eq) in DMF (3 mL) was added potassium carbonate (136.38 mg, 0.99 mmol, 1.5 eq) and BnBr (112.52 mg, 0.66 mmol, 1.0 eq) at 25° C., then the reaction mixture was stirred at this temperature for 2 hours under nitrogen atmosphere. The reaction mixture was quenched with a saturated aqueous solution of NH₄Cl (30 mL), extracted with EA (100 mL×2), washed with brine (30 mL), dried over anhydrous sodium sulfate, filtrated and the filtrate was concentrated under reduced pressure. The residue was purified by prep-TLC (PE:EA=15:1) to afford 3-(benzyloxy)-2-iodo-5-(trifluoromethyl)phenol (110.0 mg, 42% yield) as yellow oil. LC-MS: m/z 392.9, [M−H]⁻, ESI neg.

### Step E: 1-(Benzyloxy)-2-iodo-3-methoxy-5-(trifluoromethyl)benzene

To a solution of 3-(benzyloxy)-2-iodo-5-(trifluoromethyl)phenol (330.0 mg, 0.84 mmol, 1.0 eq) in DMF (6 mL) was added potassium carbonate (47.98 mg, 1.26 mmol, 1.5 eq), followed by CH₃I (237.69 mg, 1.67 mmol, 2.0 eq) dropwise and the reaction mixture was stirred at 25° C. for 1 hour under nitrogen atmosphere. The reaction mixture was quenched with a saturated aqueous solution of NH₄Cl (50 mL) at 25° C. dropwise, extracted with EA (200 mL×3), washed with brine (30 mL), dried over anhydrous sodium sulfate, filtrated and the filtrate was concentrated under reduced pressure to afford 1-(benzyloxy)-2-iodo-3-methoxy-5-(trifluoromethyl)benzene (400.0 mg, 88% yield) as a yellow solid. ¹H NMR (400 MHz, CDCl₃) δ 7.54-7.52 (m, 2H), 7.44-7.33 (m, 4H), 6.79 (s, 1H), 6.74 (s, 1H), 5.21 (s, 2H), 3.96 (s, 3H).

### Step F: 2-(2-(Benzyloxy)-6-methoxy-4-(trifluoromethyl)phenyl)-4,4,5,5-tetramethyl-1,3,2-dioxaborolane

To a solution of 1-(benzyloxy)-2-iodo-3-methoxy-5-(trifluoromethyl)benzene (200.0 mg, 0.49 mmol, 1.0 eq) and 2-isopropoxy-4,4,5,5-tetramethyl-1,3,2-dioxaborolane (266.47 mg, 1.43 mmol, 2.0 eq) in THF (3 mL) was added n-BuLi (0.43 mL, 1.07 mmol, 1.5 eq) dropwise at −60° C., then the reaction mixture was stirred at this temperature for 1 hour under nitrogen atmosphere. The reaction mixture was quenched with a saturated aqueous solution of NH₄Cl (30 mL), extracted with EA (100 mL×3), washed with brine (20 mL), the combined organic layer was dried over anhydrous sodium sulfate, filtrated and the filtrate was concentrated under reduced pressure. The residue was purified by prep-TLC (PE:EA=20:1) to afford the title compound (50.0 mg) as a yellow solid. ¹H NMR (400 MHz, CDCl₃) δ 7.46-7.43 (m, 2H), 7.36-7.31 (m, 3H), 6.79 (s, 1H), 6.72 (s, 1H), 5.07 (s, 2H), 3.83 (s, 3H), 1.31 (s, 12H).

### Step G: 3-Methoxy-2-(4, 4, 5, 5-tetramethyl-1, 3, 2-dioxaborolan-2-yl)-5-(trifluoromethyl)phenol

A solution of 2-(2-(benzyloxy)-6-methoxy-4-(trifluoromethyl)phenyl)-4,4,5,5-tetramethyl-1,3,2-dioxaborolane (30.0 mg, 0.06 mmol, 1.0 eq) in ethyl acetate (4 mL) and methanol (1 mL) was evacuated and refilled with nitrogen three times, then Pd/C (15.0 mg, 10% purity) was added. The reaction was evacuated and refilled with hydrogen three times, then the reaction mixture was stirred at 25° C. for 0.5 hour under hydrogen atmosphere (1100 mmHg). The reaction mixture was filtrated and the solid was washed with MeOH (20 mL×2), the filtrate was concentrated under reduced pressure to afford 3-methoxy-2-(4,4,5,5-tetramethyl-1,3,2-dioxaborolan-2-yl)-5-(trifluoromethyl)phenol (22.0 mg, 92% yield) as a yellow solid. ¹H NMR (400 MHz, CDCl₃) δ 8.79 (s, 1H), 6.77 (s, 1H), 6.57 (s, 1H), 3.86 (s, 3H), 1.39 (s, 12H).

### Step H: 2-[2-[[(3R)-1-Ethyl-3-piperidyl]amino]oxazolo[4,5-b]pyridin-5-yl]-3-methoxy-5-(trifluoromethyl)phenol;2,2,2-trifluoroacetic acid salt

To a solution of (R)-5-bromo-N-(1-ethylpiperidin-3-yl)oxazolo[4,5-b]pyridin-2-amine; 2,2,2-trifluoroacetic acid (20.0 mg, 0.05 mmol, 1.0 eq), 3-methoxy-2-(4,4,5,5-tetramethyl-1, 3, 2-dioxaborolan-2-yl)-5-(trifluoromethyl)phenol (18.83 mg, 0.06 mmol, 1.3 eq) and potassium carbonate (31.42 mg, 0.23 mmol, 5.0 eq) in 1, 4-dioxane (2 mL) and water (0.2 mL) was added Pd(dppf)Cl₂ (3.33 mg, 0.0 mmol, 0.1 eq). Then the reaction mixture was evacuated and refilled with nitrogen three times, then the reaction mixture was stirred at 100° C. for 3 hours under nitrogen atmosphere. After cooling to ambient temperature, the reaction mixture was purified by C18 column chromatograph (0.1% TFA in water/MeCN condition) directly and then prep-HPLC (Instrument ACSWH-GX-N, Method Column Phenomenex Synergi Polar-RP 100×25 mm×4 um, Condition water (TFA)-ACN, Begin B: 31, End B: 51. Gradient Time (min): 7, 100% B.

Hold Time (min): 2, FlowRate (m/min): 25) to afford (R)-2-(2-((1-ethylpiperidin-3-yl)amino)oxazolo[4,5-b]pyridin-5-yl)-3-methoxy-5-(trifluoromethyl)phenol as TFA salt (6.57 mg, 26% yield) as a yellow solid. LC-MS: m/z 437.1 [M+H]⁺, ESI pos.

### Example 32

### (rac)-2-[2-[(1-Ethyl-3-piperidyl)amino]oxazolo[4,5-b]pyrazin-5-yl]-3-methyl-5-(trifluoromethyl)phenol;2,2,2-trifluoroacetic acid salt

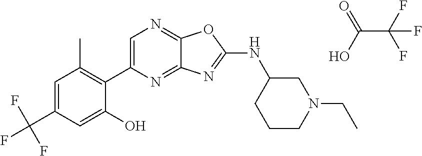 [SMILES: CCN1CCCC(Nc2nc3nc(-c4c(C)cc(C(F)(F)F)cc4O)cnc3o2)C1 ; O=C(O)C(F)(F)F]

### Step A: 5-(Trifluoromethyl)-1,2,4-triazin-3-amine

To a stirred solution of benzyl alcohol (5.19 g, 47.98 mmol, 1.0 eq) in tetrahydrofuran (100 mL) was added NaH (1.92 g, 47.98 mmol, 1.0 eq) at 20° C., The mixture was stirred at ambient temperature for 10 mins. The 3-bromo-6-chloro-pyrazin-2-amine, CAS: 212779-21-0 (10.0 g, 47.98 mmol, 1.0 eq) was then added and the reaction mixture was heated at 70° C. for 4 hours. The mixture was quenched by water 5 mL, ethyl acetate (50 mL) and water (20 mL) were added and layers were separated. The aqueous phase was extracted with ethyl acetate (50 mL×2). Combined extracts were washed with brine (60 mL), dried over Na₂SO₄, filtered, and concentrated under vacuum to give a residue which was purified by silica gel chromatography (eluting with petroleum ether:ethyl acetate=10:1 to 3:1, to give 3-benzyloxy-6-chloro-pyrazin-2-amine (3.0 g, 25% yield) as a yellow solid. LC-MS: m/z 236.0 [M+H]⁺, ESI pos.

### Step B: 2-(6-Amino-5-benzyloxy-pyrazin-2-yl)-3-methyl-5-(trifluoromethyl)phenol

The apparatus was dried by heating with a heat-gun under vacuum. To a mixture of 3-benz yloxy-6-chloro-pyrazin-2-amine (1.3 g, 5.52 mmol, 1.0 eq), CsF (2.51 g, 16.55 mmol, 3.0 eq) and 3-methyl-2-(4,4,5,5-tetramethyl-1,3,2-dioxaborolan-2-yl)-5-(trifluoromethyl)phenol (2.5 g, 8.27 mmol, 1.5 eq), in 1,4-dioxane (30 mL) and water (5 mL) was added XPhos Pd G₃ (0.47 g, 0.55 mmol, 0.1 eq) at 25° C., and the mixture was stirred at 100° C. for 16 h under N₂ to get a brown solution. Cooled to 25° C., the mixture was quenched by water 10 mL, ethyl acetate (50 mL) and water (20 mL) were added and layers were separated. The aqueous phase was extracted with ethyl acetate (50 mL×2). Combined extracts were washed with brine (60 mL), dried over Na₂SO₄, filtered, and concentrated under vacuum to give a residue which was purified by silica gel chromatography (eluting with petroleum ether:ethyl acetate=10:1 to 3:1 to obtained 2-(6-amino-5-benzyloxy-pyrazin-2-yl)-3-methyl-5-(trifluoromethyl)phenol (1.5 g, 60% yield) as a yellow solid. LC-MS: m/z 376.2 [M+H]⁺, ESI pos.

### Step C: (rac)-Benzyl 3-isothiocyanatopiperidine-1-carboxylate

To a stirring solution of TEA (862.2 mg, 8.54 mmol, 2.0 eq), benzyl 3-aminopiperidine-1-carboxylate, CAS: 711002-74-3 (1.0 g, 4.27 mmol, 1.0 eq) in dichloromethane (5 mL) at 0° C. was added a solution of thiophosgene (490.76 mg, 4.27 mmol, 1.0 eq) dropwise. And stirred for 2 h at 25° C., the mixture was concentrated under vacuum to give a residue. The residue was purified by silica gel chromatography (eluting with petroleum ether:ethyl acetate=10:1 to 3:1 to give benzyl 3-isothiocyanatopiperidine-1-carboxylate (1.0 g, 85% yield) as colorless oil. ¹H NMR (400 MHz, DMSO-d6) δ 7.43-7.28 (m, 5H), 5.19-5.04 (m, 2H), 4.06-4.00 (m, 1H), 3.94-3.66 (m, 2H), 3.40-3.38 (m, 1H), 3.20-3.04 (m, 1H), 1.94-1.83 (m, 2H), 1.68-1.49 (m, 2H).

### Step D: (rac)-3-(Benzyloxy)-6-(2-methyl-4-(trifluoromethyl)-6-((2-(trimethylsilyl)ethoxy)-methoxy)phenyl)pyrazin-2-amine

The apparatus was dried by heating with a heat-gun under vacuum. To a mixture of cesium carbonate (390.63 mg, 1.2 mmol, 1.5 eq) and 2-(6-amino-5-benzyloxy-pyrazin-2-yl)-3-methyl-5-(trifluoromethyl)phenol (300.0 mg, 0.8 mmol, 1.0 eq), in DMF (5 mL) at 25° C., and the mixture was stirred at 25° C. for 30 mins, then the SEMCl (199.8 mg, 1.2 mmol, 1.5 eq) was added and stirred at 25° C. for 2 h. the mixture was partitioned between ethyl acetate (50 mL) and water (20 mL). The organic portion concentrated to dryness under reduced pressure. The residue was purified by silica gel chromatography (eluting with petroleum ether:ethyl acetate=10:1 to 3:1 to give the title compound (200 mg, 50% yield) as colorless oil. LC-MS: m/z 506.2 [M+H]⁺, ESI pos.

### Step E: (rac)-Benzyl 3-[[3-benzyloxy-6-[2-methyl-4-(trifluoromethyl)-6-(2-trimethylsilylethoxy-methoxy)phenyl]pyrazin-2-yl]carbamothioylamino]piperidine-1-carboxylate

A stirring solution of 3-benzyloxy-6-[2-methyl-4-(trifluoromethyl)-6-(2-trimethylsilylethoxymethoxy)phenyl]pyrazin-2-amine (150.0 mg, 0.3 mmol, 1.0 eq) in tetrahydrofuran (1.5 mL) was added NaH (23.73 mg, 0.59 mmol, 60%, 2.0 eq) stirred for 0.5 h at 25° C. then the benzyl 3-isothiocyanatopiperidine-1-carboxylate (98.38 mg, 0.36 mmol, 1.2 eq) was added and stirred for 3 h at 25° C., The mixture was quenched by water 5 mL, The aqueous layer was extracted with EtOAc (50 mL×3). The combined organic layers were dried over anhydrous Na₂SO₄, filtered, and concentrated in vacuum. The residue was purified by silica gel chromatography (eluting with petroleum ether:ethyl acetate=10:1 to 3:1) to the title compound (60.0 mg, 26% yield) as a yellow solid. LC-MS: m/z 782.5 [M+H]⁺, ESI pos.

### Step F: (rac)-1-(3-Hydroxy-6-(2-hydroxy-6-methyl-4-(trifluoromethyl)phenyl)pyrazin-2-yl)-3-(piperidin-3-yl)thiourea; trifluoroacetic acid salt

A stirring solution of benzyl 3-[[3-benzyloxy-6-[2-methyl-4-(trifluoromethyl)-6-(2-trimethylsilylethoxymethoxy)phenyl]pyrazin-2-yl]carbamothioylamino]piperidine-1-carboxylate (50.0 mg, 0.06 mmol, 1.0 eq), anisole (0.07 mL, 0.64 mmol, 10.0 eq) and TFA (1.0 mL) was stirred for 4 h at 75° C. The reaction mixture was concentrated under reduced pressure. The residue was purified by C18 column chromatography (20 g, 0.1% TFA in water/MeCN), then the eluent was lyophilized to afford 1-[3-hydroxy-6-[2-hydroxy-6-methyl-4-(trifluoromethyl) phenyl]pyrazin-2-yl]-3-(3-piperidyl)thiourea; TFA salt (30.0 mg, 99% yield) as a yellow solid. LC-MS: m/z 428.2 [M+H]⁺ (ESI pos).

### Step G: (rac)-1-(1-Ethyl-3-piperidyl)-3-[3-hydroxy-6-[2-hydroxy-6-methyl-4-(trifluoromethyl) phenyl]pyrazin-2-yl]thiourea; trifluoroacetic acid salt

To a solution of 1-[3-hydroxy-6-[2-hydroxy-6-methyl-4-(trifluoromethyl)phenyl]pyrazin-2-yl]-3-(3-piperidyl)thiourea; TFA salt (25.0 mg, 0.05 mmol, 1.0 eq), acetic acid (3.33 mg, 0.06 mmol, 1.2 eq) in methanol (1 mL) was added acetaldehyde (0.03 mL, 0.23 mmol, 40%, 5.0 eq) and stirred at 25° C. for 0.5 h, then the NaBH₃CN (14.51 mg, 0.23 mmol, 5.0 eq) was added and stirred for 1.5 h at 25° C. The reaction mixture was concentrate d under reduced pressure. The residue was purified by C18 column chromatography (20 g, 0.1% TFA in water/MeCN), then the eluent was lyophilized to afford 1-(1-ethyl-3-piperidyl)-3-[3-hydroxy-6-[2-hydroxy-6-methyl-4-(trifluoromethyl)phenyl]pyrazin-2-yl] thiourea; TFA salt (25.0 mg, 94% yield) as a yellow solid. LC-MS: m/z 456.3 [M+H]⁺, ESI pos.

### Step H: (rac)-2-[2-[(1-Ethyl-3-piperidyl)amino]oxazolo[4,5-b]pyrazin-5-yl]-3-methyl-5-(trifluoromethyl)phenol;2,2,2-trifluoroacetic acid salt

A mixture of 1-(1-ethyl-3-piperidyl)-3-[3-hydroxy-6-[2-hydroxy-6-methyl-4-(trifluoromethyl) phenyl]pyrazin-2-yl]thiourea (TFA salt) (15.0 mg, 0.03 mmol, 1.0 eq), tetrabutylazanium;iodide (9.73 mg, 0.03 mmol, 1.0 eq) in tetrahydrofuran (0.5 mL) was added hydrogen peroxide (5.97 mg, 0.05 mmol, 2.0 eq) and stirred at 25° C. for 2 hours, then reaction mixture was concentrated under reduced pressure. The residue was purified by C18 column chromatography (20 g, 0.1% TFA in water/MeCN), then the eluent was lyophilized to afford the title compound (10.2 mg, 70% yield) as a yellow solid. LC-MS: m/z 422.2 [M+H]⁺, ESI pos.

### Example 33

### 3-Methyl-2-[7-methyl-2-[[(3R)-1-methyl-3-piperidyl]amino]oxazolo[4,5-b]pyridin-5-yl]-5-(trifluoromethyl)phenol;2,2,2-trifluoroacetic acid salt

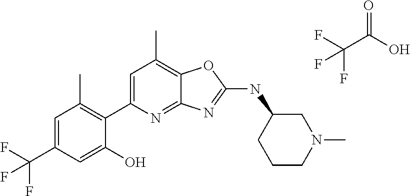 [SMILES: Cc1cc(C(F)(F)F)cc(O)c1-c1cc(C)c2oc(N[C@@H]3CCCN(C)C3)nc2n1 ; O=C(O)C(F)(F)F]

### Step A: 6-Chloro-4-methyl-2-nitropyridin-3-ol

The apparatus was dried by heating with a heatgun under vacuum. In a three round bottom flask, 6-chloro-4-methyl-pyridin-3-ol (CAS: 1227502-89-7) (800 mg, 5.57 mmol, 1.0 eq) was added to H₂SO₄ (5.46 g, 55.72 mmol, 10.0 eq) at 0° C., then a mixture of fuming nitric acid (657.0 mg, 10.43 mmol, 1.87 eq) and H₂SO₄ (1.09 g, 11.14 mmol, 2.0 eq) was added to the mixture and the mixture was stirred at 0° C. for 1 hour, then the mixture was stirred at 25° C. for 12 hours. The reaction mixture was quenched by 20 mL ice water, then extracted by EtOAc (30 ml×3), washed with brine (20 ml×2), dry over anhydrous Na₂SO₄, filtrated the filtrate was concentrated under reduce pressure. The residue was purified by column silica gel column chromatography (PE:EA=20:1 to 10:1). To give the desired product 6-chloro-4-methyl-2-nitropyridin-3-ol (1200.0 mg) was obtained as a yellow solid. ¹H NMR (400 MHz, DMSO-d₆): δ 10.95 (brs, 1H), 7.77 (s, 1H), 2.32 (s, 3H).

### Step B: 2-Amino-6-chloro-4-methylpyridin-3-ol

To a solution of 6-chloro-4-methyl-2-nitropyridin-3-ol (500.0 mg, 2.65 mmol, 1.0 eq) in THF (15 mL) and water (3 mL) was added Fe (1.48 g, 26.52 mmol, 10.0 eq) and NH₄Cl (1.42 g, 26.52 mmol, 10.0 eq) portion wise, then the reaction mixture was stirred at 70° C. for 2 hours under nitrogen atmosphere. The reaction was cooled to room temperature, filtrated and the filtrate was concentrated under reduced pressure. The residue was purified by column silica gel column chromatography (PE:EtOAc:THF=10:1:1) to afford the title compound (150.0 mg, 36% yield) as a yellow solid. LC-MS: m/z 159.0 [M+H]⁺, ESI pos.

### Step C: 5-Chloro-7-methyloxazolo[4,5-b]pyridine-2-thiol

To a solution of 2-amino-6-chloro-4-methylpyridin-3-ol (150.0 mg, 0.95 mmol, 1.0 eq) in N,N-dimethylformamide (3 mL) was added di(1H-imidazol-1-yl)methanethione (303.4 mg, 1.7 mmol, 1.8 eq) portionwise and the reaction mixture was stirred at 20° C. for 12 hours under nitrogen atmosphere. The reaction was quenched with 20 mL of water, extracted with EtOAc (100 mL×2), washed with 1N HCl (20 mL×2) aqueous solution, brine (20 mL), dried over Na₂SO₄, filtrated and the filtrate was concentrated under reduced pressure to give 5-chloro-7-methyloxazolo[4,5-b]pyridine-2-thiol (180.0 mg, 71% yield) as a yellow solid, which was used for next step without further purification. LC-MS: m/z 200.9, [M+H]⁺, ESI pos.

### Step D: 2,5-Dichloro-7-methyloxazolo[4,5-b]pyridine

To a mixture of 5-chloro-7-methyloxazolo[4,5-b]pyridine-2-thiol (150.0 mg, 0.75 mmol, 1.0 eq) in oxalyl chloride (4.74 g, 37.38 mmol, 50.0 eq) was added DMF (5.46 mg, 0.07 mmol, 0.1 eq), and the mixture was stirred at 50° C. for 1 hour. The reaction mixture was concentrated in vacuum. The residue was added in to ice water (20 mL) at 0° C. and extracted with EtOAc (30 mL×2). Combined extracts were washed with brine (60 mL), dried over Na₂SO₄, filtered, and concentrated under vacuum to give a residue. The residue was purified by column silica gel column chromatography (hexane/EtOAc, 5:1), to give the title (40.0 mg, 26% yield) as a white solid. LC-MS: m/z 203.0, [M+H]⁺, ESI pos.

### Step E: (R)-5-Chloro-7-methyl-N-(1-methylpiperidin-3-yl)oxazolo[4,5-b]pyridin-2-amine

To a solution of 2,5-dichloro-7-methyloxazolo[4,5-b]pyridine (40.0 mg, 0.2 mmol, 1.0 eq) in NMP (2 mL) was added (R)-1-methylpiperidin-3-amine (67.49 mg, 0.6 mmol, 3.0 eq) and DIEA (127.07 mg, 0.99 mmol, 5.0 eq). The mixture was stirred at 20° C. for 2 hour. The mixture was purified by C18 column chromatography (0.1% TFA in water/MeCN condition). The eluent was lyophilized to give the desired product (R)-5-chloro-7-methyl-N-(1-methylpiperidin-3-yl)oxazolo[4,5-b]pyridin-2-amine (60.0 mg, 77% yield, TFA salt) as a yellow solid. LC-MS: m/z 281.1, [M+H]⁺, ESI pos.

### Step F: 3-Methyl-2-[7-methyl-2-[[(3R)-1-methyl-3-piperidyl]amino]oxazolo[4,5-b]pyridin-5-V]-5-(trifluoromethyl)phenol;2,2,2-trifluoroacetic acid salt

To a solution of 3-methyl-2-(4,4,5,5-tetramethyl-1,3,2-dioxaborolan-2-yl)-5-(trifluoromethyl)phenol (36.73 mg, 0.12 mmol, 1.2 eq) and (R)-5-chloro-7-methyl-N-(1-methylpiperidin-3-yl)oxazolo[4,5-b]pyridin-2-amine (40.0 mg, 0.1 mmol, 1.0 eq) in 1,4-dioxane (1.5 mL) and Water (0.3 mL) was added CsF (61.56 mg, 0.41 mmol, 4.0 eq) and Xphos Pd G3 (17.17 mg, 0.02 mmol, 0.2 eq) under nitrogen atmosphere. The mixture was stirred at 100° C. for 3 hours. The mixture was concentrated in vacuum, the residue was purified by prep-HPLC (Method: Column: Phenomenex Synergi Polar-RP 100×25 mm×4 pin; Condition: water (TFA)-MeCN, Begin B: 27; End B: 47; Gradient Time (min): 7; 100% B Hold Time (min): 2; FlowRate (mL/min): 25.). The eluent was lyophilized to give the title compound (19.4 mg, 34% yield, TFA salt) as yellow solid. LC-MS: m/z 421.1, [M+H]⁺ (ESI pos).

### Example 34

### 5-Methoxy-3-methyl-2-[2-[[(3R)-1-methyl-3-piperidyl]amino]oxazolo[4,5-b]pyridin-5-yl]phenol

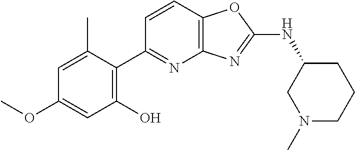 [SMILES: COc1cc(C)c(-c2ccc3oc(N[C@@H]4CCCN(C)C4)nc3n2)c(O)c1]

### Step A: 2-Iodo-5-methoxy-3-methyl-phenol

To a stirred solution of 3-methoxy-5-methylphenol (1.0 g, 7.24 mmol, 1.0 eq) (CAS: 3209-13-0) in toluene (42 mL) at rt was added sodium hydride, 60% in oil (614.0 mg, 15.35 mmol, 2.12 eq) and the reaction was left to stir for 1 h. After this time, iodine (1.86 g, 7.33 mmol, 1.01 eq) was added and the reaction was stirred for 4 h. The reaction mixture was carefully poured onto a mixture of ice-cold water (90 mL) and 1M HCl (10 mL) and stirred for 10 min. The resulting solution was diluted with water (50 mL) and DCM (50 mL). The separated aqueous layer was further extracted with DCM (2×50 mL) and the combined organic layers dried (Na₂SO₄), filtered and concentrated. The crude reaction mixture was purified by column chromatography on silica gel (40 g, 25-100% DCM:isoHexanes) to afford the title compound (779.0 mg, 40% yield) as a white crystalline solid. LC-MS m/z 264.9 [M+H]⁺, ESI pos.

### Step B: 1-Benzyloxy-2-iodo-5-methoxy-3-methyl-benzene

A suspension of potassium carbonate (833.0 mg, 6.03 mmol, 3.0 eq) in acetone (30 mL) was sonicated for 5 min then 2-iodo-5-methoxy-3-methyl-phenol (530.0 mg, 2.01 mmol, 1.0 eq) was added and the mixture was sonicated for further 5 min before the addition of benzyl bromide (265.0 uL, 2.23 mmol, 1.11 eq). The resulting stirred mixture was heated at reflux for 2.5 h and then cooled to r.t. and concentrated. The residue was dissolved in DCM (50 mL) and washed with 50 v % brine (100 mL). The aqueous layer was re-extracted with DCM (2×50 mL), and the combined organic layers were dried (MgSO₄), filtered and concentrated. The crude product was purified by column chromatography on silica gel (40 g, 0-50% DCM:isoHexanes) to afford the title compound (628.0 mg, 86% yield) as a colourless oil. LC-MS m/z 355.2 [M+H]⁺, ESI pos.

### Step C: 2-(2-Benzyloxy-4-methoxy-6-methyl-phenyl)-4,4,5,5-tetramethyl-1,3,2-dioxaborolane

To a stirred solution of 1-benzyloxy-2-iodo-5-methoxy-3-methyl-benzene (256.0 mg, 0.72 mmol, 1.0 eq) and 2-isopropoxy-4,4,5,5-tetramethyl-1,3,2-dioxaborolane (165.0 μL, 0.81 mmol, 1.12 eq) in THF (5 mL) at −78° C. was added n-BuLi (2.5 M in hexanes, 350 μL, 0.88 mmol, 1.21 eq) and the reaction was stirred for 2 h. Additional n-BuLi (2.5 M in hexanes, 350.0 μL, 0.88 mmol, 1.21 eq) and 2-isopropoxy-4,4,5,5-tetramethyl-1,3,2-dioxaborolane (165.0 uL, 0.81 mmol, 1.12 eq) were added and the reaction was stirred for 30 min. The reaction mixture was warmed to ˜0° C. and quenched with sat. NH4Cl (5 mL) then diluted with water (45 mL) and DCM (50 mL) and separated. The separated aqueous layer was further extracted with DCM (2×50 mL). The combined organic layers were dried (Na₂SO₄), filtered and concentrated. The crude product was purified by column chromatography on silica gel (40 g, 0-20% MTBE:isoHexanes) to afford the title compound (133 mg, 50% yield) as a colourless oil. LC-MS m/z 355.6 [M+H]⁺, ESI pos.

### Step D: 5-(2-Benzyloxy-4-methoxy-6-methyl-phenyl)-N-[(3R)-1-methyl-3-piperidyl]oxazolo-[4,5-b]pyridin-2-amine

2-(2-Benzyloxy-4-methoxy-6-methyl-phenyl)-4,4,5,5-tetramethyl-1,3,2-dioxaborolane (67.0 mg, 0.19 mmol, 0.84 eq), 5-bromo-N-[(3R)-1-methyl-3-piperidyl]oxazolo[4,5-b]pyridin-2-amine (Example 30, step H) (70.0 mg, 0.22 mmol, 1.0 eq) and potassium carbonate (61.0 mg, 0.44 mmol, 1.96 eq) in 1,4-dioxane (4 mL) and water (1 mL) was sparged (bubbling nitrogen for 10 min whilst sonicating). Xphos Pd G3 (9.0 mg, 0.01 mmol, 0.05 eq) and XPhos (3.0 mg, 0.01 mmol, 0.03 eq) were added and the reaction mixture was stirred at 90° C. for 4 h. The reaction was cooled to rt and additional Xphos Pd G3 (10.0 mg, 0.01 mmol, 0.05 eq), XPhos (3.0 mg, 0.01 mmol, 0.03 eq) and potassium carbonate (58.0 mg, 0.42 mmol, 1.87 eq) were added and the reaction mixture was stirred at 90° C. for 16 h. The reaction was cooled and degassed before the addition of 2-(2-benzyloxy-4-methoxy-6-methyl-phenyl)-4,4,5,5-tetramethyl-1,3,2-dioxaborolane (66.0 mg, 0.19 mmol, 0.83 eq) in 1,4-dioxane (0.5 mL) followed by Xphos Pd G3 (5.0 mg, 0.01 mmol, 0.03 eq) and XPhos (2.0 mg, 0.0 mmol, 0.02 eq) and the reaction was heated at 90° C. for 2 h. The reaction mixture was cooled to r.t, concentrated and the crude was purified by chromatography on silica gel (40 g, 0-20% MeOH (0.7 M NH-3):EtOAc) and then by reverse phase column chromatography (C₁₈, 43 g, 10-60% Acetonitrile (0.1% formic acid):water (0.1% formic acid)) to afford the title compound (18.0 mg, 17% yield) as a brown solid. LC-MS m/z 459.4 [M+H]⁺, ESI pos.

### Step E: 5-Methoxy-3-methyl-2-[2-[[(3R)-1-methyl-3-piperidyl]amino]oxazolo[4,5-b]pyridin-5-yl]phenol

A stirred solution of 5-(2-benzyloxy-4-methoxy-6-methyl-phenyl)-N-[(3R)-1-methyl-3-piperidyl]oxazolo[4,5-b]pyridin-2-amine (17.0 mg, 0.04 mmol, 1.0 eq) and Pd/C (Type 87) (6.0 mg, 0.0 mmol, 0.08 eq) in EtOAc (4 mL) was placed in a hydrogenation vessel under 1 bar of H2. The reaction was left to stirred at r.t. for 32 h. The reaction mixture was diluted with EtOH (1 mL), and additional Pd/C (Type 87) (6.0 mg, 0.0 mmol, 0.08 eq) was added and the reaction was placed in a hydrogenation vessel under 1 bar of H2 an additional 19 h. The reaction mixture was filtered through celite, the filter cake rinsed with EtOH (50 mL) and concentrated to afford the title compound (6.1 mg, 39% yield) as a light brown solid. LC-MS m/z 369.3 [M+H]⁺, ESI pos.

### Example 35

### 5-[2-[[(3R)-1-Ethyl-3-piperidyl]amino]-6-methyl-oxazolo[4,5-b]pyridin-5-yl]-2,3-dihydrobenzofuran-4-ol

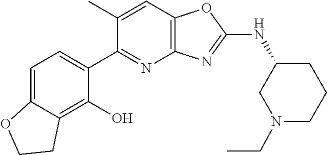 [SMILES: CCN1CCC[C@@H](Nc2nc3nc(-c4ccc5c(c4O)CCO5)c(C)cc3o2)C1]

### Step A: 6-Bromo-5-chloro-3H-oxazolo[4,5-b]pyridine-2-thione

To a solution of commercially available 2-amino-5-bromo-6-chloro-pyridin-3-ol (CAS #1131041-72-9, 1.06 g, 4.74 mmol, 1.00 eq) in N,N-dimethylformamide (13.7 mL) under argon atmosphere was added 1,1′-thiocarbonyldiimidazole (972.2 mg, 5.46 mmol, 1.15 eq). The reaction mixture was stirred at room temperature overnight. Then, potassium carbonate (1.31 g, 9.49 mmol, 2.00 eq) was added followed by methyl iodide (343 μL, 5.48 mmol, 1.16 eq). The reaction mixture was stirred at room temperature for 2 h for completion. The reaction mixture was cooled to 0° C. and ˜30 mL water was added dropwise (a precipitate was formed) and was further stirred at 0° C. for 30 min. The suspension was filtered and rinsed with water to afford the desired product (850 mg, 61% yield) as light brown solid. LC-MS: m/z 280.9 [M+H]⁺, ESI pos.

### Step B: 6-Bromo-5-chloro-N-[(3R)-1-ethyl-3-piperidyl]oxazolo[4,5-b]pyridin-2-amine

To a solution of aforementioned 6-bromo-5-chloro-2-(methylthio)oxazolo[4,5-b]pyridine (CAS #1783370-92-2, 850 mg, 2.89 mmol, 1.00 eq) in 1,4-dioxane (6.1 mL) was added [(3R)-1-ethyl-3-piperidyl]amine (411.1 mg, 457.8 μL, 3.21 mmol, 1.11 eq) followed by triethylamine (327.4 mg, 450.9 μL, 3.24 mmol, 1.12 eq). The orange solution was stirred at 90° C. overnight. LC-MS showed the reaction was complete. The reaction mixture was cooled to room temperature and extracted with ˜40 mL ethyl acetate and ˜5 mL saturated NaHCO₃-solution. The aqueous layer was back extracted with ˜40 mL ethyl acetate. The organic layers were washed with ˜5 mL water and ˜5 mL brine. The combined organic layers were dried over sodium sulfate, filtered and concentrated in vacuo. The crude product was purified by flash chromatography (silica gel, 25 g, DCM, MeOH 0-20% MeOH) to afford the title compound (691 mg, 63% yield) as a light brown solid. LC-MS: m/z 359.1 [M+H]⁺, ESI pos.

### Step C: 5-Chloro-N-[(3R)-1-ethyl-3-piperidyl]-6-methyl-oxazolo[4,5-b]pyridin-2-amine

To a solution of aforementioned (6-bromo-5-chloro-N-[(3R)-1-ethyl-3-piperidyl]oxazolo[4,5-b]pyridin-2-amine (698 mg, 1.94 mmol, 1.00 eq) in N,N-dimethylformamide (11.7 mL) were added under argon commercially available tributyl(methyl)stannane (CAS #1528-01-4, 769.7 mg, 706.2 μL, 2.52 mmol, 1.30 eq), PdCl₂(PPh₃) (136.2 mg, 0.194 mmol, 0.100 eq). The reaction was stirred at 100° C. for one hour. LC-MS showed only starting material so that additional PdCl₂(PPh₃) (136.0 mg, 0.194 mmol, 0.10 eq) was added to the reaction mixture. The mixture was stirred at 100° C. for four hours until completion. The reaction mixture was filtered over dicalite and washed with DCM. The crude product was adsorbed on ISOLUTE HM-N and purified by flash chromatography (silica gel, 25 g, DCM/MeOH 0-20% MeOH). Product and corresponding debrominated product could not be separated. All fractions containing product were combined and concentrated in vacuo to afford (460 mg) as brown solid. LC-MS: m/z 281.1[M+H]⁺, ESI pos.

### Step D: 5-Bromo-2,3-dihydrobenzofuran-4-ol

To a solution of coumaran-4-ol (5.0 g, 36.7 mmol, 1.0 eq) in methanol (100 mL) was added portionwise pyridinium tribromide (12.3 g, 38.6 mmol, 1.05 eq) at −50° C. to −40° C. The reaction mixture was stirred at −50° C. to −40° C. for 30 min, then warmed to rt and stirred overnight. The reaction mixture was extracted with DCM and 1M aq. HCl-solution. The organic layer was washed with 1M aq. HCl-solution and brine. The aqueous layers were backextracted three times with DCM. The combined organic layers were dried over sodium sulfate, filtered and concentrated in vacuo. The crude product was purified by flash chromatography (silica gel, 220 g, gradient 0% to 20% ethyl acetate in heptane) to afford the title compound (4.59 g, 55% yield) as off-white solid. LC-MS: m/z=214.9/216.9 (Br isotopes) [M+H]⁺, ESI pos.

### Step E: 4-Benzyloxy-5-bromo-2,3-dihydrobenzofuran

To a solution of 5-bromo-2,3-dihydrobenzofuran-4-ol (4.59 g, 20.26 mmol, 1.0 eq) in acetonitrile (40 mL) was added potassium carbonate (5.6 g, 40.51 mmol, 2.0 eq) followed by benzyl bromide (4.89 g, 3.4 mL, 28.57 mmol, 1.4 eq). The reaction mixture was stirred at rt for 2 h. The reaction mixture was extracted with ethyl acetate (140 mL) and water (30 mL). The aqueous layer was backextracted with ethyl acetate (140 mL). The organic layers were washed with water (20 mL) and brine (20 mL). The combined organic layers were dried over sodium sulfate, filtered and concentrated in vacuo. The crude product was adsorbed on ISOLUTE HM-N and purified by flash chromatography (silica gel, 220 g, gradient 0% to 10% ethyl acetate in heptane) to afford the title compound (6.17 g, 95% yield) as colorless oil. LC-MS: m/z=305.1/307.0 (Br isotopes) [M+H]⁺, ESI pos.

### Step F: 2-(4-Benzyloxy-2,3-dihydrobenzofuran-5-yl)-4,4,5,5-tetramethyl-1,3,2-dioxaborolane

To a solution of 4-benzyloxy-5-bromo-2,3-dihydrobenzofuran (6.16 g, 19.18 mmol, 1.0 eq) and 2-isopropoxy-4,4,5,5-tetramethyl-1,3,2-dioxaborolane (5.47 g, 6 mL, 29.41 mmol, 1.5 eq) in THF (80 mL) was added dropwise 1.6 M n-butyllithium, solution in hexane (19 mL, 30.4 mmol, 1.6 eq) within 40 min at −76° C. Let stir at −76° C. for 2.5 h. The reaction mixture was warmed to −60° C., quenched with saturated aq. NH4Cl-solution (40 mL) at −60° C., warmed to rt and then extracted with ethyl acetate (300 mL) and saturated aq. NH₄Cl-solution (40 mL). The aqueous layer was backextracted with ethyl acetate (300 mL). The organic layers were washed with brine (80 mL). The combined organic layers were dried over sodium sulfate, filtered and concentrated in vacuo. The crude product was adsorbed on ISOLUTE HM-N and purified by flash chromatography (silica gel, 120 g, gradient 0% to 10% ethyl acetate in heptane) to afford the title compound (5.78 g, 81% yield) as colorless oil. LC-MS: m/z=353.1 [M+H]⁺, ESI pos.

### Step G: 5-(4,4,5,5-Tetramethyl-1,3,2-dioxaborolan-2-yl)-2,3-dihydrobenzofuran-4-ol

A solution of 2-(4-benzoxycoumaran-5-yl)-4,4,5,5-tetramethyl-1,3,2-dioxaborolane (5.77 g, 15.6 mmol, 1.0 eq) in ethyl acetate (70 mL) was three times alternating evacuated and flushed with argon. Then palladium on activated charcoal, 10% Pd basis (577 mg, 0.542 mmol, 0.035 eq) was added carefully. The reaction flask was evacuated, flushed with argon, evacuated and flushed with hydrogen. The reaction mixture was stirred under hydrogen atmosphere (balloon) at room temperature for 3 h. The LC/MS showed still starting material remaining, and methanol (10 mL) was added. The reaction flask was three times alternating evacuated and flushed with argon, evacuated and then flushed with hydrogen. The reaction mixture was stirred under hydrogen atmosphere (balloon) at room temperature for 1 h. Afterwards, the reaction mixture was filtered and rinsed well with ethyl acetate/methanol. The filtrate was concentrated in vacuo to afford the title compound (4.22 g, 98% yield) as off-white solid. LC-MS: m/z=263.2 [M+H]⁺, ESI pos.

### Step H: 5-[2-[[(3R)-1-Ethyl-3-piperidyl]amino]-6-methyl-oxazolo[4,5-b]pyridin-5-yl]-2,3-dihydrobenzofuran-4-ol

To a solution of 5-(4,4,5,5-tetramethyl-1,3,2-dioxaborolan-2-yl)coumaran-4-ol (106.7 mg, 0.407 mmol, 1.50 eq) in 1,4-dioxane (1.85 mL), were added [1,1′-Bis(diphenylphosphino)ferrocene]dichloropalladium(II), complex with dichloromethane (26.6 mg, 0.033 mmol, 0.120 eq), and aforementioned mixture of 5-Chloro-N-[(3R)-1-ethyl-3-piperidyl]-6-methyl-oxazolo[4,5-b]pyridin-2-amine contaminated with corresponding debrominated compound (156.2 mg, 0.271 mmol, 1.00 eq) and water (0.923 mL). The reaction was flushed with argon for 5 min and then potassium carbonate (168.8 mg, 1.22 mmol, 4.50 eq) was added. The reaction was stirred at 100° C. over night. After complete conversion, the reaction mixture was cooled to room temperature and extracted with ethyl acetate and water. The aqueous layer was back extracted with ethyl acetate two times. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane). Product and by-product could not be separated. All fractions containing product were combined and concentrated in vacuo. The crude material (60 mg) was given to prep HPLC (YMC-Triart Cis, 12 nm, 5 μm, 100×30 mm, ACN/Water+0.1% TEA) to afford the title compound (10 mg, 9%) as an off-white solid and the side product 5-[2-[[(3R)-1-ethyl-3-piperidyl]amino]oxazolo[4,5-b]pyridin-5-yl]coumaran-4-ol (25 mg, 23% yield) as grey solid. LC-MS (title compound) m/z 393.2 [M−H]⁻, ESI neg.

### Example 36

### 5-Cyclopropyl-3-methyl-2-[2-[[(3R)-1-methyl-3-piperidyl]amino]oxazolo[4,5-b]pyridin-5-yl]phenol

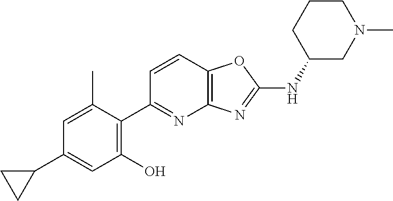 [SMILES: Cc1cc(C2CC2)cc(O)c1-c1ccc2oc(N[C@@H]3CCCN(C)C3)nc2n1]

### Step A: 3-Cyclopropyl-5-methyl-phenol

A mixture of 3-bromo-5-methylphenol (5.0 g, 26.73 mmol, 1.0 eq), K₂CO₃ (7.39 g, 53.47 mmol, 2.0 eq) and cyclopropylboronic acid (6.89 g, 80.2 mmol, 3.0 eq) in 1, 4-dioxane (50 mL) and water (10 mL) was evacuated and refilled with N₂ three times and Pd(dppf)Cl₂ (1956.16 mg, 2.67 mmol, 0.1 eq) was added to the mixture. The mixture was stirred at 100° C. for 12 hours. The mixture was poured into 1N HCl (200 mL), then, extracted with ethyl acetate (40 mL×3), the combined organic layers were washed with brine (200 mL), dried over anhydrous sodium sulfate, filtered and the filtrate was concentrated under reduced pressure, the residue was purified by silica gel column chromatography (SiO₂, Petroleum ether/Ethyl acetate=1/0 to 10/1) and purified by C18 column chromatograph (0.1% TFA in water/MeCN). After lyophilisation, 3-cyclopropyl-5-methyl-phenol (1.7 g, 43% yield) was obtained as yellow oil. ¹H NMR (400 MHz, CD₃OD) δ 6.37 (s, 2H), 6.27 (s, 1H), 2.20 (s, 3H), 1.80-1.72 (m, 1H), 0.91-0.84 (m, 2H), 0.62-0.57 (m, 2H).

### Step B: 5-Cyclopropyl-2-iodo-3-methyl-phenol

To a mixture of 3-cyclopropyl-5-methyl-phenol (1.20 g, 8.1 mmol, 1.0 eq) in Toluene (100 mL) was added NaH (388.66 mg, 16.19 mmol, 60% w/w, 2.0 eq) at 0° C. under N₂, stirred for 30 minutes, then 12 (1.64 g, 6.48 mmol, 0.8 eq) was added and stirred for 15.5 hours at 25° C. The mixture was poured into 1N HCl (100 mL) and extracted with ethyl acetate (60 mL×3). The combined organic layers were washed with brine (50 mL), dried over anhydrous sodium sulfate, filtered, and the filtrate was concentrated under reduced pressure. The residue was purified by silica gel column chromatography, petroleum ether/Ethyl acetate=10/1 and purified by C₁₈ column chromatograph (0.1% TFA in water/MeCN). After lyophilization 2-iodo-3, 5-dimethyl-phenol (6.9 g, 34% yield) was obtained as yellow solid. ¹H NMR (400 MHz, CD₃OD) δ 6.53 (d, 1H), 6.36 (d, 1H), 2.35 (s, 3H), 1.77 (dd, 1H), 0.94-0.89 (m, 2H), 0.65-0.57 (m, 2H)

### Step C: 1-Benzyloxy-5-cyclopropyl-2-iodo-3-methyl-benzene

To a solution of 5-cyclopropyl-2-iodo-3-methyl-phenol (350.0 mg, 1.28 mmol, 1.0 eq) in DMF (7 mL) was added K₂CO₃ (352.94 mg, 2.55 mmol, 2.0 eq) portion wise, followed by benzyl bromide (0.46 mL, 3.83 mmol, 3.0 eq) was added dropwise and the reaction mixture was stirred at 25° C. for 2 h under nitrogen atmosphere. The reaction was quenched with water (50 mL), extracted with EA (100 mL×3), washed with brine (20 mL), the organic layer was dried over Na₂SO₄, filtrated and the filtrate was concentrated under reduced pressure. The residue was purified by silica gel column chromatography (SiO₂, Petroleum ether/Ethyl acetate=20:1) to afford 1-benzyloxy-5-cyclopropyl-2-iodo-3-methyl-benzene (450 mg) as colourless oil. ¹H NMR (400 MHz, CD₃OD) δ 7.42 (d, 2H), 7.29 (d, 2H), 7.22 (d, 1H), 6.55 (d, 1H), 6.38 (d, 1H), 5.01 (s, 2H), 2.29 (s, 3H), 1.78-1.68 (m, 1H), 0.88-0.81 (m, 2H), 0.80-0.74 (m, 2H), 0.58-0.51 (m, 2H)

### Step D: 2-(2-Benzyloxy-4-cyclopropyl-6-methyl-phenyl)-4,4,5,5-tetramethyl-1,3,2-dioxaborolane

To a mixture of 1-benzyloxy-5-cyclopropyl-2-iodo-3-methyl-benzene (250.0 mg, 0.69 mmol, 1.0 eq) and 2-isopropoxy-4,4,5,5-tetramethyl-1,3,2-dioxaborolane (229.88 mg, 1.24 mmol, 1.8 eq) in THF (3 mL) and was added nBuLi (0.41 mL, 1.03 mmol, 1.5 eq) dropwise under N2 for 1 h. Then, the mixture was stirred at −60° C. for 30 mins. The mixture was poured into NH₄Cl solution (15 mL) and extracted with ethyl acetate (20 mL×3). The combined organic layers were washed with brine (20 mL), dried over anhydrous sodium sulfate, filtered, and the filtrate was concentrated under reduced pressure. The residue was purified by silica gel column chromatography, petroleum ether/Ethyl acetate=50/1 to give 2-(2-benzyloxy-4-cyclopropyl-6-methyl-phenyl)-4,4,5,5-tetramethyl-1,3,2-dioxaborolane (250.0 mg) as colorless oil. ¹H NMR (400 MHz, CD₃OD) δ 7.47 (d, 2H), 7.38-7.25 (m, 3H), 6.49 (d, 2H), 5.00 (s, 2H), 2.27 (s, 3H), 1.84 (dd, 1H), 1.27 (s, 12H), 0.96-0.84 (m, 3H), 0.68-0.62 (m, 2H).

### Step E: 5-(2-Benzyloxy-4-cyclopropyl-6-methyl-phenyl)-N-[(3R)-1-methyl-3-piperidyl]oxazolo-[4,5-b]pyridin-2-amine

A mixture of 2-(2-benzyloxy-4-cyclopropyl-6-methyl-phenyl)-4,4,5,5-tetramethyl-1,3,2-dioxaborolane (105.36 mg, 0.29 mmol, 2.0 eq), K₂CO₃ (39.97 mg, 0.29 mmol, 2.0 eq) and 5-bromo-N-[(3R)-1-methyl-3-piperidyl] oxazolo[4,5-b] pyridin-2-amine (45.0 mg, 0.14 mmol, 1.0 eq) in 1,4-dioxane (1 mL) and water (0.2 mL) was degassed and purged with N2 three times and Pd(dppf)Cl₂ (10.58 mg, 0.01 mmol, 0.1 eq) was added to the mixture. The mixture was stirred at 90° C. for 2 hrs. The reaction was quenched with water (20 mL), extracted with EA (20 mL×3), washed with brine (20 mL), the organic layer was dried over Na₂SO₄, filtrated and the filtrate was concentrated under reduced pressure to afford the title compound (30.0 mg, 36% yield) as yellow oil. LC-MS: m/z 469.3 [M+H]⁺, ESI pos.

### Step F: 5-Cyclopropyl-3-methyl-2-[2-[[(3R)-1-methyl-3-piperidyl]amino]oxazolo[4,5-b]pyridin-5-yl]phenol

To a solution of 5-(2-benzyloxy-4-cyclopropyl-6-methyl-phenyl)-N-[(3R)-1-methyl-3-piperidyl] oxazolo[4,5-b] pyridin-2-amine (15.0 mg, 0.03 mmol, 1.0 eq) in DCM (0.5 mL) and BBr₃ (0.5 mL, 0.6 mmol, 4.39 eq) was added to the mixture. The mixture was stirred at −65° C. for 1 h. First add an ice cube to the reaction solution, and then add methanol slowly to quench the reaction. Finally, ammonia is used to adjust the pH. The mixture was concentrated under reduced pressure and purified by reversed-phase flash (0.1% TFA) and purified by reversed-phase flash (0.1% TFA) and prep-HPLC (Method Column 3_Phenomenex Luna C18 75×30 mm×3 um Condition water (TFA)-ACN Begin B 16 End B 36 Gradient Time (min) 8; 100% B Hold Time (min) 2; Flow Rate (mL/min) 25; Injections 1 HPLC 99). After lyophilisation, the title compound was obtained as a yellow solid (2.77 mg, 5% yield). LC-MS: m/z 379.2 [M+H]⁺, ESI pos.

### Example 37a and 37b

### 2-[2-[(3aS,7aR)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol or 2-[2-[(3aR,7aS)-6-methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol and 2-[2-[(3aR,7aS)-6-methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)-phenol or 2-[2-[(3aS,7aR)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol

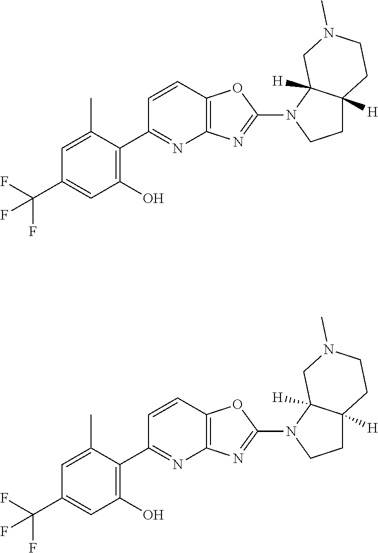

In a sealed tube, to a yellow suspension of 5-chloro-2-(6-methyl-3,3a,4,5,7,7a-hexahydro-2H-pyrrolo[2,3-c]pyridin-1-yl)oxazolo[4,5-b]pyridine (Example 20, step 1) (70 mg, 237 μmol, 1.00 eq) and 3-methyl-2-(4,4,5,5-tetramethyl-1,3,2-dioxaborolan-2-yl)-5-(trifluoromethyl)phenol (CAS #2557358-06-0, 107.3 mg, 88.7 μL, 355 μmol, 1.50 eq) in 1,4-dioxane (2.4 mL) and water (0.60 mL) was added under stirring cesium carbonate (231 mg, 710 μmol, 3.00 eq) at room temperature followed by the addition of XPhos Pd G3 (20.0 mg, 23.7 μmol, 0.10 eq) under an atmosphere of argon. After, argon was bubbled through the yellow reaction mixture for 3 minutes. The dark-yellow reaction mixture was heated to 100° C. (oil bath) for 16 hours. The black reaction mixture was cooled to room temperature and extracted with ethyl acetate twice (2×˜50 mL). The organic layers were washed with water (˜20 mL) and brine (˜20 mL). The aqueous layers were reextracted with ethyl acetate (˜40 mL). The combined organic extracts were dried over sodium sulfate, filtered off and concentrated in vacuo. The crude product was absorbed on ISOLUTE HM-N and purified by flash chromatography (silica gel; 0%-10% methanol in dichlormethane) to afford the title compound (86 mg, 76%) as light brown foam which was directly submitted to chiral separation (column chiral IJ, 5 μm, 250×20 mm, flow: 90 m/min; SFC). LC-MS: m/z 433.2 [M+H]⁺, ESI pos; to afford the first fraction 37a (39 mg, 43% yield) and the second fraction 37b (37 mg, 40% yield) both as light brown foam. LCMS m/z: 433.2 433.2 [M+H]⁺, ESI pos.

### Example 38

### (rac)-3-Methyl-2-[2-(tetrahydrofuran-3-ylamino)oxazolo[4,5-b]pyridin-5-yl]-5-(trifluoromethyl)phenol

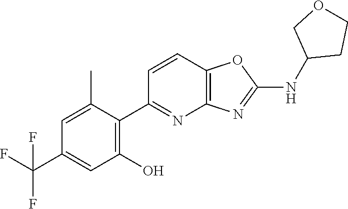

### Step A: 5-Chloro-N-tetrahydrofuran-3-yl-oxazolo[4,5-b]pyridin-2-amine

To a solution of 5-chloro-2-(methylthio)oxazolo[4,5-b]pyridine (CAS #1783370-92-2, 131 mg, 0.62 mmol, 1.0 eq) in 1,4-dioxane, extra dry (2 mL) was added commercially available tetrahydrofuran-3-ylamine (CAS #88675-24-5, 64.8 mg, 63.6 μL, 0.744 mmol, 1.20 eq) followed by triethylamine (81.6 mg, 112 μL, 0.806 mmol, 1.30 eq). The light brown solution was stirred at 90° C. for 16 hours. The reaction mixture was cooled to room temperature and extracted with ethyl acetate (30 mL) and saturated NaHCO₃-solution (15 mL). The aqueous layer was back extracted with ethyl acetate (30 mL). The organic layers were washed with water (10 mL) and brine (10 mL). The combined organic layers were dried over sodium sulfate, filtered and concentrated in vacuo. The residue was purified by flash chromatography (SiO₂; 0-90% ethyl acetate in heptane) to give the title compound (133 mg, 90%) as white solid. LC-MS: m/z 240.1; 242.1 (Cl isotopes) [M+H]⁺, ESI pos

### Step B: (rac)-3-Methyl-2-[2-(tetrahydrofuran-3-ylamino)oxazolo[4,5-b]pyridin-5-yl]-5-(trifluoromethyl)phenol

In a sealable tube, a mixture of aforementioned (5-chlorooxazolo[4,5-b]pyridin-2-yl)-tetrahydrofuran-3-yl-amine (40 mg, 0.167 mmol, 1.00 eq), [2-hydroxy-6-methyl-4-(trifluoromethyl)phenyl]boronic acid (CAS #2557358-06-0, 51.4 mg, 0.234 mmol, 1.40 eq) and potassium carbonate (103.8 mg, 0.751 mmol, 4.50 eq) was stirred in 1,4-dioxane (2.29 mL) and water (1.14 mL). Argon was bubbled through the mixture for 2 min, then the catalyst 1,1′-bis(diphenylphosphino)ferrocene-palladium(ii)dichloride dichloromethane complex (16.4 mg, 0.02 mmol, 0.12 eq) was added finally. The sealed tube was stirred at 90° C. for 16 hrs. The reaction mixture was extracted with ethyl acetate (2×20 mL) and half-saturated NH₄Cl-solution (20 mL). The organic layers were washed with water (20 mL) and brine (20 mL). The combined organic layers were dried over sodium sulfate, filtered and concentrated in vacuo. The residue (112 mg) was submitted to preparative RP HPLC (column: YMC-triart C₁₈, 12 nm, 5 um, 100×30 mm; CAN/water+0.1% HCOOH) to give the title compound (5 mg, 8%) as white amorph freeze-dried solid. LC-MS: m/z 380.2 [M+H]⁺, ESI pos.

### Example 39, 40 and 41

### 5-Chloro-3-methyl-2-[2-[[rac-(8S,8aR)-1,2,3,5,6,7,8,8a-octahydroindolizin-8-yl]amino]oxazolo[4,5-b]pyridin-5-yl]phenol, 2-[2-[[(8R,8aS)-1,2,3,5,6,7,8,8a-Octahydroindolizin-8-yl]amino]oxazolo[4,5-b]pyridin-5-yl]-5-chloro-3-methyl-phenol and 2-[2-[[(8S,8aR)-1,2,3,5,6,7,8,8a-octahydroindolizin-8-yl]amino]oxazolo[4,5-b]pyridin-5-yl]-5-chloro-3-methyl-phenol

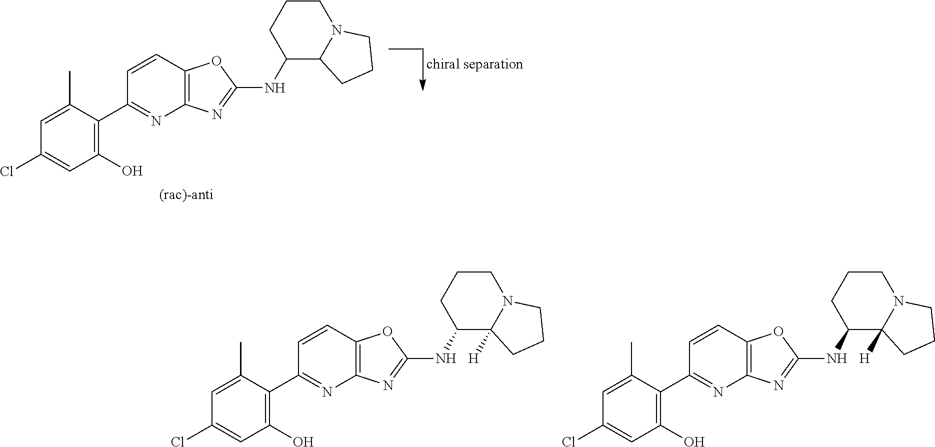

### Step A: 5-Chloro-N-[rac-(8S,8aR)-1,2,3,5,6,7,8,8a-octahydroindolizin-8-yl]oxazolo[4,5-b]pyridin-2-amine

To a solution of aforementioned 5-chloro-2-(methylthio)oxazolo[4,5-b]pyridine (CAS #1783370-92-2, 150 mg, 0.710 mmol, 1.00 eq) in 1,4-dioxane (1.5 mL) was added [rac-(8R,8aS)-indolizidin-8-yl]amine (CAS #1993250-73-9, 115.9 mg, 0.785 mmol, 1.106 eq) followed by triethylamine (111 μL, 0.796 mmol, 1.121 eq). The light brown solution was stirred at 90° C. overnight. Reaction mixture was stirred at 100° C. for another 4 hrs. A precipitate was formed, which was trituration in ether to give the desired product as a white solid (105 mg, 51%). LC-MS: m/z 293.2 [M+H⁺, ESI pos.

### Step B: 2-[2-(1,2,3,5,6,7,8,8a-octahydroindolizin-8-ylamino)oxazolo[4,5-b]pyridin-5-yl]-5-chloro-3-methyl-phenol (rac anti)

A mixture of aforementioned 5-chloro-N-[rac-(8S,8aR)-1,2,3,5,6,7,8,8a-octahydroindolizin-8-yl]oxazolo[4,5-b]pyridin-2-amine (step A) (105 mg, 0.359 mmol, 1.00 eq), (4-chloro-2-hydroxy-6-methyl-phenyl)boronic acid (CAS #1207961-50-9, 96.3 mg, 0.516 mmol, 1.440 eq) 1.44 eq), cesium carbonate (172.9 mg, 0.531 mmol, 1.48 eq) and Xphos-Pd-G3 (31.3 mg, 0.037 mmol, 0.103 eq) in 1,4-dioxane (1.5 mL) and water (0.375 mL) was flushed with argon and stirred at 100° C. for 2 h. The reaction mixture was cooled to room temperature and extracted with ˜30 mL ethyl acetate and ˜5 mL water. The aqueous layer was backextracted with ˜30 mL ethyl acetate. The organic layers were washed with ˜5 mL water and ˜5 mL brine. The combined organic layers were dried over sodium sulfate, filtered and concentrated in vacuo. The crude product was adsorbed on ISOLUTE HM-N and purified by flash chromatography (silica gel, gradient 0% to 10% (methanol+2% NH₄OH) in dichloromethane) to give the title compound as a light yellow powder (34 mg). LC-MS: m/z 397.3 [M−H]⁻, ESI neg.

### Step C: 2-[2-[[(8R,8aS)-1,2,3,5,6,7,8,8a-Octahydroindolizin-8-yl]amino]oxazolo[4,5-b]pyridin-5-yl]-5-chloro-3-methyl-phenol or 2-[2-[[(8S,8aR)-1,2,3,5,6,7,8,8a-octahydroindolizin-8-yl]amino] oxazolo[4,5-b]pyridin-5-yl]-5-chloro-3-methyl-phenol and 2-[2-[[(8S,8aR)-1,2,3,5,6,7,8,8a-octahydroindolizin-8-yl]amino]oxazolo[4,5-b]pyridin-5-yl]-5-chloro-3-methyl-phenol or 2-[2-[[(8R,8aS)-1,2,3,5,6,7,8,8a-Octahydroindolizin-8-yl]amino]oxazolo[4,5-b]pyridin-5-yl]-5-chloro-3-methyl-phenol

The mix fractions were purified by RP HPLC (SFC, 20-40% MeOH+0.2% DEA, column AD-H) to give two fractions: the first one (example 41) eluting at rt=3.11 min, (11.7 mg, 100% ee) and the second (example 40) eluting at rt=3.97 min (13 mg, 100% ee) both as a light brown solid. LC-MS: m/z 397.3 [M−H]⁻, ESI neg.

### Example 42

### 5-Chloro-3-methyl-2-[2-(6-methyl-3,4a,5,7,8,8a-hexahydro-2H-pyrido[4,3-b][1,4]oxazin-4-yl)oxazolo[4,5-b]pyridin-5-ylphenol

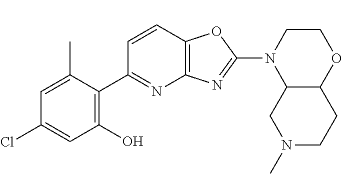

### Step A: 4-(5-chlorooxazolo[4,5-b]pyridin-2-yl)-6-methyl-3,4a,5,7,8,8a-hexahydro-2H-pyrido[4,3-b][1,4]oxazine

To a mixture of 5-chloro-2-(methylthio)oxazolo[4,5-b]pyridine (CAS #1783370-92-2, 242 mg, 1.21 mmol, 1.00 eq) and 6-methyl-2,3,4,4a,5,7,8,8a-octahydropyrido[4,3-b][1,4]oxazine (245 mg, 1.57 mmol, 1.30 eq) in 1,4-dioxane, extra dry (4 mL) and N-methyl-2-pyrrolidinone, extra dry (2 mL) was added at room temperature triethylamine (244 mg, 336 μL, 2.41 mmol, 2.00 eq). This reaction mixture was stirred in a sealed tube at 110° C. for 16 hours. Reaction was not complete, so that the temperature was increased to 150° C. and stirring was continued for 4 hours. The reaction mixture was quenched with water (10 mL) and extracted with ethylacetate (2×30 mL). The organic layers were washed with water (30 mL) and brine (30 mL). The combined organic extracts were dried over sodium sulfate, filtered off and concentrated in vacuo. The residue was purified by flash chromatography (silica gel, 0-50% ethyl acetate in heptane; then ethyl acetate:methanol 9:1 (v/v) to afford the title compound (233 mg, 63%) as light brown foam. LC-MS: m/z 309.1 ([{35C₁}M+H]+), 311.1 ([{37Cl}M+H]+), ESI pos.

### Step B: 5-Chloro-3-methyl-2-[2-(6-methyl-3,4a,5,7,8,8a-hexahydro-2H-pyrido[4,3-b][1,4]oxazin-4-yl)oxazolo[4,5-b]pyridin-5-yl]phenol

In a sealable tube, a mixture of aforementioned 4-(5-chlorooxazolo[4,5-b]pyridin-2-yl)-6-methyl-3,4a,5,7,8,8a-hexahydro-2H-pyrido[4,3-b][1,4]oxazine (73 mg, 0.236 mmol, 1.00 eq), (4-chloro-2-hydroxy-6-methyl-phenyl)boronic acid (CAS #1207961-50-9, 88.1 mg, 0.473 mmol, 2.00 eq), and potassium carbonate (147 mg, 1.06 mmol, 4.50 eq) in 1,4-dioxane (2 mL) and water (1 mL) was stirred and argon was bubbled through the mixture for 2 min, then the catalyst 1,1′-bis(diphenylphosphino)ferrocene-palladium(ii)dichloride dichloromethane complex (28.9 mg, 0.035 mmol, 0.15 eq) was added finally. The sealed tube was stirred at 100° C. for 16 hours. The reaction mixture was extracted with ethyl acetate (2×20 mL) and half-saturated NH₄Cl-solution (20 mL). The organic layers were washed with water (30 mL) and brine (30 mL). The combined organic layers were dried over sodium sulfate, filtered and concentrated in vacuo. The residue (110 mg) was purified by flash chromatography (silica gel, gradient 0% to 100% (dichloromethane:methanol:NH₄OH 110:10:1) in dichloromethane) followed by further purification on preparative HPLC to afford the title compound (4 mg, 4%) as white amorph freeze-dried solid. LC-MS: m/z 413.1 ([{35C₁}M−H]+), 415.1 ([{37Cl}M−H]⁺), ESI neg.

### Example 43

### 2-[2-(2-Azabicyclo[2.1.1]hexan-1-ylmethylamino)oxazolo[4,5-b]pyridin-5-yl]-3-methyl-5-(trifluoromethyl)phenol;dihydrochloride

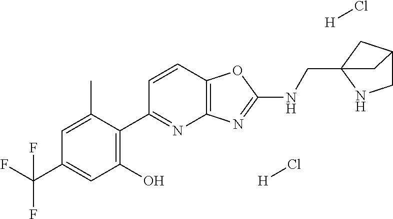

### Step A: tert-butyl 1-[[(5-Chlorooxazolo[4,5-b]pyridin-2-yl)amino]methyl]-2-azabicyclo[2.1.1]hexane-2-carboxylate

To a mixture of 5-chloro-2-(methylthio)oxazolo[4,5-b]pyridine (CAS #1783370-92-2, 111 mg, 553.2 umol, 1.000 eq) and triethylamine (112 mg, 154 μL, 1.11 mmol, 2.00 eq) in 1,4-dioxane, extra dry (1 mL) and N-methyl-2-pyrrolidinone, extra dry (1 mL) was added at room temperature tert-butyl 1-(aminomethyl)-2-azabicyclo[2.1.1]hexane-2-carboxylate (153 mg, 719 μmol, 1.30 eq). This reaction mixture was stirred in a sealed tube at 120° C. for 16 hours. The temperature was increased to 150° C. and stirring was continued for 2 hours until full conversion. The reaction mixture was quenched with water (10 mL) and extracted with ethylacetate (2×30 mL). The organic layers were washed with water (30 mL) and brine (30 mL). The combined organic extracts were dried over sodium sulfate, filtered off and concentrated in vacuo. The residue was purified by flash chromatography (silica gel, 0-50% ethyl acetate in heptane) to give the title compound (156 mg, 77.29%) as light yellow solid. LC-MS: m/z 365.2 ([{35C₁}M+H]+), 367.2 ([{37Cl}M+H]+), ESI pos.

### Step B: tert-butyl 1-[[[5-[2-hydroxy-6-methyl-4-(trifluoromethyl)phenyl]oxazolo[4,5-b]pyridin-2-yl]amino]methyl]-2-azabicyclo[2.1.1]hexane-2-carboxylate

A mixture of aforementioned tert-butyl 1-[[(5-Chlorooxazolo[4,5-b]pyridin-2-yl)amino]methyl]-2-azabicyclo[2.1.1]hexane-2-carboxylate (154 mg, 422 μmol, 1.00 eq), 3-methyl-2-(4,4,5,5-tetramethyl-1,3,2-dioxaborolan-2-yl)-5-(trifluoromethyl)phenol (283.4 mg, 844 μmol, 2.000 eq) and potassium carbonate (280 mg, 2.03 mmol, 4.80 eq) was dissolved in 1,4-dioxane (4 mL) and water (2 mL). The sealable tube was flushed with argon and 1,1′-bis(diphenylphosphino)ferrocene-palladium(ii)dichloride dichloromethane complex (51.7 mg, 63.3 μmol, 0.15 eq) was added. Flushed again with argon and the sealed tube was stirred at 100° C. (oil bath) for 16 hrs. Under argon methanesulfonato(2-dicyclohexylphosphino-2′,4′,6′-tri-1-propyl-1,1′-biphenyl)(2′-amino-1,1′-biphenyl-2-yl)palladium(ii) (XPhos Pd G3) (71.5 mg, 84.4 μmol, 0.20 eq) and cesium carbonate (137.5 mg, 422 μmol, 1.00 eq) were added and stirring continued at 110° C. (oil bath) for another 4 hours. The reaction mixture was cooled to room temperature and quenched with water (20 mL) and sat NH₄Cl sol (20 mL), then extracted with dichlormethane (3×30 mL). Organic layers were washed with brine (40 mL), dried over Na₂SO₄, filtered off and concentrated in vacuo. The residue was purified by flash chromatography (SiO₂; 0-50% ethyl acetate in heptane) followed by trituration in heptane to give the title compound (91 mg, 43%) as white solid. LC-MS: m/z 505.3 [M+H]⁺, ESI pos.

### Step C: 2-[2-(2-Azabicyclo[2.1.1]hexan-1-ylmethylamino)oxazolo[4,5-b]pyridin-5-yl]-3-methyl-5-(trifluoromethyl)phenol;dihydrochloride

To a solution of aforementioned tert-butyl 1-[[[5-[2-hydroxy-6-methyl-4-(trifluoromethyl)phenyl]oxazolo[4,5-b]pyridin-2-yl]amino]methyl]-2-azabicyclo[2.1.1]hexane-2-carboxylate (89 mg, 176 μmol, 1.00 eq) in dichloromethane (4 mL) and methanol (2 mL) was added at room temperature dropwise 4 M HCl in dioxane (441 μL, 1.76 mmol, 10.0 eq). The reaction mixture was stirred at 23° C. for 20 hours. The reaction mixture was concentrated in vacuo followed by crystallisation with EtOH/EA/heptane to afford the title compound as a 1:2 hydrochloride (74 mg, 83%) as white solid. LC-MS: m/z 403.2 [M−H]⁻, ESI neg.

### Example 44

### 5-Chloro-3-methyl-2-[2-[[(3R)-1-methyl-3-piperidyl]amino]oxazolo[4,5-b]pyridin-5-yl]phenol

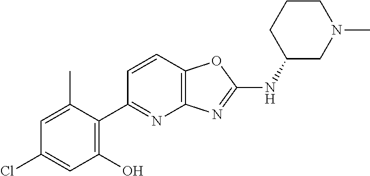 [SMILES: Cc1cc(Cl)cc(O)c1-c1ccc2oc(N[C@@H]3CCCN(C)C3)nc2n1]

The title compound was obtained as a light brown powder, LCMS: m/z 373.2 [M+H]⁺, ESI pos, using chemistry similar to that described in Example 19, starting from 5-chloro-N-[(3R)-1-methyl-3-piperidyl]oxazolo[4,5-b]pyridin-2-amine (Example 15, step 1) and (4-chloro-2-hydroxy-6-methyl-phenyl)boronic acid (CAS #1207961-50-9).

### Example 45

### 2-[2-[[(3R)-1-Ethyl-3-piperidyl]amino]oxazolo[4,5-b]pyridin-5-yl]-5-fluoro-3-methyl-phenol

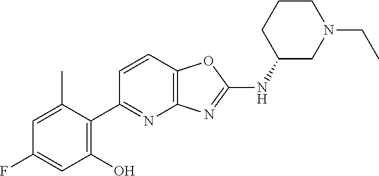 [SMILES: CCN1CCC[C@@H](Nc2nc3nc(-c4c(C)cc(F)cc4O)ccc3o2)C1]

The title compound was obtained as a light yellow solid, LCMS: m/z 371.3 [M+H]⁺, ESI pos, using chemistry similar to that described in Example 19, starting from 5-chloro-N-[(3R)-1-ethyl-3-piperidyl]oxazolo[4,5-b]pyridin-2-amine (Example 14, step 1) and (4-fluoro-2-hydroxy-6-methyl-phenyl)boronic acid intermediate A (see preparation below).

### Intermediate A: (4-Fluoro-2-hydroxy-6-methyl-phenyl)boronic acid

### Step A: 2-Bromo-4-fluoro-6-methyl-aniline

To a solution of 4-fluoro-2-methyl-aniline (CAS #452-71-1, 200 g, 1.60 mol) in DMF (3000 mL) was cooled to −10° C., then N-bromosuccinimide (313 g, 1.76 mol, 1.1 eq) was added in portions below 30° C. After addition, the mixture was stirred at 25° C. for 3 hrs. The reaction mixture was dissolved in H₂O (6000 mL) and then extracted with EtOAc (2000 mL*2). The organic layers were washed with brine (1000 mL), dried over Na₂SO₄ and concentrated under reduced pressure to give the title compound (230 g, 71% yield) as a purple liquid. LCMS m/z 204.0, [M+H]⁺, ESI pos.

### Step B: 4-Fluoro-2-methoxy-6-methyl-aniline

To a solution of aforementioned 2-bromo-4-fluoro-6-methyl-aniline (230 g, 1.13 mol) in MeOH (460 mL) was added sodium methylate (1150 mL, 5.75 mol) and copper iodide (236 g, 1.24 mol). Then the mixture was stirred at 65° C. for 4 hrs. The mixture was quenched with NH₄Cl (aq. sat. 3000 mL), then filtered and the solution was extracted with EtOAc (2000 mL*2). The organic layer was washed with brine (1000 mL), dried over Na₂SO₄ and concentrated under reduced pressure to give a residue. The residue was purified by chromatography (silica gel, petroleum ether:Ethyl acetate=10:1 to 5:1 to give the title compound (127 g, 73% yield) as a yellow oil. ¹H NMR (400 MHz, CDCl₃): δ=6.47 (td, 2H), 3.84 (s, 3H) 2.17 (s, 3H).

### Step C: 5-Fluoro-2-iodo-1-methoxy-3-methyl-benzene

To a mixture of tetrafluoroboric acid (317 g, 1.44 mol, 40% in water) and water (160 mL) was added aforementioned 4-fluoro-2-methoxy-6-methyl-aniline (80.0 g, 516 mmol), then the mixture was cooled to 0˜ 5° C. and EtOH (80.0 mL) was added. Then a mixture of sodium nitrite (39.1 g, 567 mmol) in water (80.0 mL) was added dropwise to the reaction solution at 0˜5° C. After addition, the mixture was stirred for 0.5 hr at 0-5° C. The mixture was filtered, the filter cake was collected and dissolved in water (400 mL). Then a solution of potassium iodide (128 g, 773 mmol) in water (400 mL) was added dropwise at 10˜20° C. After addition, the reaction mixture was stirred at 20˜30° C. for 1 h. The mixture was extracted with EtOAc (500 mL*2), then the organic layers were dried over Na₂SO₄ and concentrated under reduced pressure. The residue was purified by column chromatography (silica gel, petroleum ether:Ethyl acetate=1:0 to 10:1) to give the title compound (62.6 g, 46% yield) as a light yellow oil. ¹H NMR (400 MHz, CDCl₃): δ=6.59-6.73 (m, 1H), 6.42 (dd, 1H), 3.86 (s, 3H) 2.46 (s, 3H).

### Step D: 2-(4-Fluoro-2-methoxy-6-methyl-phenyl)-4,4,5,5-tetramethyl-1,3,2-dioxaborolane

To a solution of aforementioned 5-fluoro-2-iodo-1-methoxy-3-methyl-benzene (70.0 g, 263 mmol) and 2-isopropoxy-4,4,5,5-tetramethyl-1,3,2-dioxaborolane (97.9 g, 526 mmol) in THF (350 mL) was cooled to −20° C. under N₂, then Isopropylmagnesium Chloride-Lithium Chloride (243 mL, 316 mmol) was added below −10° C. After addition, the reaction mixture was warmed to 20˜ 30° C. and stirred for 1 h. The mixture was quenched with aq. sat. NH₄Cl (350 mL), and extracted with EtOAc (300 mL*2), the organic layers were dried over Na₂SO₄ and concentrated under reduced pressure to give a residue. The residue was slurred with n-heptane (140 mL) at 0˜10° C. for 0.5 h, then filtered and the filter cake was dried in vacuum to give the title compound (46.0 g, 173 mmol, 66% yield) as an off-white solid. ¹H NMR (400 MHz, CDCl₃): δ=6.46 (dd, 1H), 6.37 (dd, 1H), 3.74 (s, 3H), 2.34 (s, 3H), 1.38 (s, 12H).

### Step E: (4-Fluoro-2-hydroxy-6-methyl-phenyl)boronic acid

To a solution of aforementioned 2-(4-fluoro-2-methoxy-6-methyl-phenyl)-4,4,5,5-tetramethyl-1,3,2-dioxaborolane (46.0 g, 173 mmol) in dichloromethane (460 mL) was cooled to −20˜ 0° C., then boron tribromide (86.6 g, 346 mmol) was added below 0° C. The reaction mixture was stirred at −20˜ 0° C. for 0.5 h. The reaction mixture was quenched with MTBE (120 mL) below 0° C. Then the mixture was filtered and the filter cake was collected and slurred with H₂O (80 mL) at 15˜ 25° C. for 0.5 h. Then the mixture was filtered and the filter cake was washed with H₂O (20 mL). The filter cake was dried at 30˜ 35° C. in vacuum. The residue slurred with EtOAc (40 mL) and n-heptane (80 mL) at 20-30° C. for 0.5 hr, then filtered and the filter cake was dried in vacuum at 30˜35° C. to give the title compound (13.2 g, 45% yield) as a white solid. ¹H NMR (400 MHz, CDCl₃): δ=6.40 (dd, 1H), 6.30 (dd, 1H), 2.20 (s, 3H).

### Example 46

### 5-Chloro-3-methyl-2-[2-[[(3R)-1-propyl-3-piperidyl]amino]oxazolo[4,5-b]pyridin-5-yl]phenol

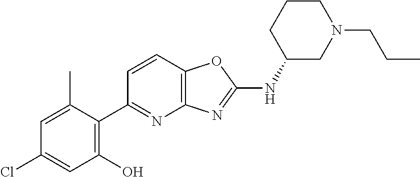 [SMILES: CCCN1CCC[C@@H](Nc2nc3nc(-c4c(C)cc(Cl)cc4O)ccc3o2)C1]

### Step 1: tert-Butyl N-[(3R)-1-propyl-3-piperidyl]carbamate

To a solution of tert-butyl N-[(3R)-3-piperidyl]carbamate (CAS #309956-78-3, 1.00 g, 4.99 mmol, 1.00 eq) in tetrahydrofuran (20 mL) was added N,N-diisopropylethylamine (1.63 g, 2.2 mL, 12.6 mmol, 2.52 eq) followed by dropwise addition of 1-iodopropane (1.01 g, 0.580 mL, 5.95 mmol, 1.19 eq) The solution was stirred at 40° C. for 16 hours. The reaction mixture was cooled to room temperature, quenched with saturated aq. NaHCO₃-solution and then extracted with ethyl acetate. The aqueous layer was backextracted twice with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, gradient 0% to 10% methanol in dichloromethane) to afford the title compound (1.14 g, 89% yield) as a light yellow solid. LCMS: m/z 243.2 [M+H]⁺, ESI pos.

### Step 2: (3R)-1-Propylpiperidin-3-amine hydrochloride

To a solution of tert-butyl N-[(3R)-1-propyl-3-piperidyl]carbamate (Example 46, step 1) (320 mg, 1.25 mmol, 1.00 eq) in dichloromethane (3.2 mL) and methanol (1.6 mL) was added dropwise 4 M HCl in dioxane (3.0 mL, 12.0 mmol, 9.57 eq). The reaction mixture was stirred at room temperature for 1 hour. The reaction mixture was concentrated in vacuo to afford the title compound (408 mg, 91% yield, 50% purity) as a light yellow oil. LCMS: m/z 143.2 [M+H]⁺, ESI pos.

### Step 3: 5-Chloro-N-[(3R)-1-propyl-3-piperidyl]oxazolo[4,5-b]pyridin-2-amine

To a mixture of 5-chloro-2-(methylthio)oxazolo[4,5-b]pyridine (CAS #1783370-92-2, 220 mg, 1.04 mmol, 1.00 eq) in 1,4-dioxane (2.0 mL) was added (3R)-1-propylpiperidin-3-amine hydrochloride (Example 46, step 2) (402 mg, 1.12 mmol, 1.08 eq, 50% purity) followed by triethylamine (366 mg, 0.504 mL, 3.62 mmol, 3.47 eq). The reaction mixture was stirred at 90° C. for 16 hours. The reaction mixture was cooled to room temperature and extracted with ethyl acetate and saturated NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in dichloromethane) to afford the title compound (274 mg, 85% yield) as a light yellow solid. LCMS: m/z 295.1 [M+H]⁺, ESI pos.

### Step 4: 5-Chloro-3-methyl-2-[2-[[(3R)-1-propyl-3-piperidyl]amino]oxazolo[4,5-b]pyridin-5-yl]phenol

The title compound was obtained as a light yellow foam, LCMS: m/z 401.2 [M+H]⁺, ESI pos, using chemistry similar to that described in Example 19, starting from 5-chloro-N-[(3R)-1-propyl-3-piperidyl]oxazolo[4,5-b]pyridin-2-amine (Example 46, step 3) and (4-chloro-2-hydroxy-6-methyl-phenyl)boronic acid (CAS #1207961-50-9).

### Example 47

### 5-Chloro-2-[2-[[(3R)-1-cyclobutyl-3-piperidyl]amino]oxazolo[4,5-b]pyridin-5-yl]-3-methyl-phenol

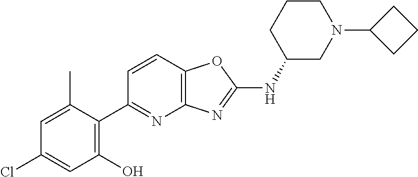 [SMILES: Cc1cc(Cl)cc(O)c1-c1ccc2oc(N[C@@H]3CCCN(C4CCC4)C3)nc2n1]

### Step 1: tert-Butyl N-[(3R)-1-cyclobutyl-3-piperidyl]carbamate

To a solution of cyclobutanone (CAS #1191-95-3, 1.05 g, 14.98 mmol, 1.50 eq) in dichloromethane (22 mL) was added tert-butyl N-[(3R)-3-piperidyl]carbamate (CAS #309956-78-3, 2.00 g, 9.99 mmol, 1.00 eq), sodium acetate (901 mg, 10.98 mmol, 1.10 eq) and acetic acid (661 mg, 0.630 mL, 11.0 mmol, 1.10 eq). Sodium triacetoxyborohydride (2.75 g, 12.98 mmol, 1.30 eq) was added in three portions and the reaction mixture was stirred at room temperature for 16 hours. The reaction mixture was basified with saturated aq. NaHCO₃-solution and saturated aq. Na₂CO₃-solution and then extracted three times with dichloromethane. The combined organic layers were dried over sodium sulfate, filtered and concentrated in vacuo. The crude product was adsorbed on ISOLUTE HM-N and purified by flash chromatography (silica gel, 40 g, gradient 0% to 5% methanol in dichloromethane) to afford the title compound (1.92 g, 72% yield) as an off-white solid. LCMS: m/z 255.3 [M+H]⁺, ESI pos.

### Step 2: 5-Chloro-N-[(3R)-1-cyclobutyl-3-piperidyl]oxazolo[4,5-b]pyridin-2-amine

To a solution of tert-butyl N-[(3R)-1-cyclobutyl-3-piperidyl]carbamate (Example 47, step 1) (204 mg, 0.76 mmol, 1.15 eq) in dichloromethane (5.6 mL) was added dropwise trifluoroacetic acid (1.63 g, 1.1 mL, 14.28 mmol, 21.5 eq). Let stir at room temperature for 3 hours. The reaction mixture was concentrated in vacuo. The residue was taken up in 1,4-dioxane (0.800 mL) and triethylamine (363 mg, 0.500 mL, 3.59 mmol, 5.41 eq) was added followed by 5-chloro-2-(methylthio)oxazolo[4,5-b]pyridine (CAS #1783370-92-2, 140 mg, 0.66 mmol, 1.00 eq). The brown solution was stirred at 90° C. for 16 hours. Triethylamine (181.5 mg, 0.250 mL, 1.79 mmol, 2.71 eq) was added at room temperature and the reaction mixture was stirred at 100° C. for 5 hours. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 10% methanol in dichloromethane) to afford the title compound (121 mg, 57% yield) as a light yellow solid. LCMS: m/z 307.1 [M+H]⁺, ESI pos.

### Step 3: 5-Chloro-2-[2-[[(3R)-1-cyclobutyl-3-piperidyl]amino]oxazolo[4,5-b]pyridin-5-yl]-3-methyl-phenol

The title compound was obtained as a light brown powder, LCMS: m/z 413.2 [M+H]⁺, ESI pos, using chemistry similar to that described in Example 19, starting from 5-chloro-N-[(3R)-1-cyclobutyl-3-piperidyl]oxazolo[4,5-b]pyridin-2-amine (Example 47, step 2) and (4-chloro-2-hydroxy-6-methyl-phenyl)boronic acid (CAS #1207961-50-9).

### Example 48

### 5-[2-[[(3R)-1-Ethyl-3-piperidyl]amino]oxazolo[4,5-b]pyridin-5-yl]-6-methyl-2,3-dihydrobenzofuran-4-ol

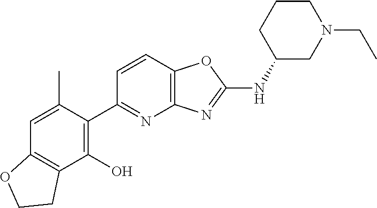 [SMILES: CCN1CCC[C@@H](Nc2nc3nc(-c4c(C)cc5c(c4O)CCO5)ccc3o2)C1]

### Step 1: 1-(2-Benzyloxy-6-hydroxy-4-methyl-phenyl)ethanone

To a solution of 1-(2,6-dihydroxy-4-methyl-phenyl)ethanone (CAS #1634-34-0, 5.1 g, 30.69 mmol, 1.00 eq) in N,N-dimethylformamide (35 mL) was added potassium carbonate (4.38 g, 31.69 mmol, 1.03 eq) followed by benzyl bromide (5.46 g, 3.8 mL, 31.95 mmol, 1.04 eq). The reaction mixture was stirred at 60° C. for 16 hours. The reaction mixture was cooled to room temperature and extracted with saturated aq. NH₄Cl-solution and three times dichloromethane. The organic layers were washed with brine. The combined organic layers were dried over sodium sulfate, filtered and concentrated in vacuo. The residue was adsorbed on ISOLUTE HM-N and purified by flash chromatography (silica gel, 220 g, gradient 5% to 20% ethyl acetate in heptane) to afford the title compound (4.94 g, 50% yield, 80% purity) as a yellow solid. LCMS: m/z 257.1 [M+H]⁺, ESI pos.

### Step 2: 4-Benzyloxy-6-methyl-benzofuran-3-one

To a solution of 1-(2-benzyloxy-6-hydroxy-4-methyl-phenyl)ethanone (Example 48, step 1) (2.81 g, 8.77 mmol, 1.00 eq, 80% purity) in tetrahydrofuran (40 mL) at −76° C. was added chlorotrimethylsilane (2.91 g, 3.4 mL, 26.79 mmol, 3.05 eq) followed by dropwise addition of 1 M lithium bis(trimethylsilyl)amide, solution in tetrahydrofuran/ethylbenzene (18.92 g, 22 mL, 22 mmol, 2.51 eq), keeping the internal temperature below −65° C. After the addition was complete, the ice bath was removed and the reaction mixture was stirred at room temperature for 2.5 hours. The reaction mixture was cooled to 0° C. and N-bromosuccinimide (1.90 g, 10.68 mmol, 1.22 eq) was added in three portions at 0° C. Let stir at 0° C. for 20 minutes and at room temperature for 1 hour and then added 1 M aq. NaOH-solution (10 mL, 10 mmol, 1.14 eq) and stirred at room temperature for 1 hour. Poured into ice cold 1 M HCl-solution and extracted with ethyl acetate. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 80 g, gradient 0% to 20% ethyl acetate in heptane) to afford the title compound (1.63 g, 66% yield, 90% purity) as a yellow solid. LCMS: m/z 255.1 [M+H]⁺, ESI pos.

### Step 3: 4-Benzyloxy-6-methyl-2,3-dihydrobenzofuran-3-ol

To a partial solution of 4-benzyloxy-6-methyl-benzofuran-3-one (Example 48, step 2) (1.05 g, 3.71 mmol, 1.00 eq, 90% purity) in methanol (10 mL) and tetrahydrofuran (5 mL) was added sodium borohydride (422 mg, 11.15 mmol, 3.00 eq) in three portions at 0° C. The reaction mixture was stirred at 0° C. for 3 hour. The reaction mixture was quenched with water and then extracted three times with dichloromethane. The organic layers were washed with brine. The combined organic layers were dried over sodium sulfate, filtered and concentrated in vacuo to afford the title compound (1.03 g, 87% yield, 80% purity) as a light brown oil, which was used without further purification. LCMS: m/z 239.1 [M−H₂O+H], ESI pos.

### Step 4: 6-Methyl-2,3-dihydrobenzofuran-4-ol

An autoclave was charged with 4-benzyloxy-6-methyl-2,3-dihydrobenzofuran-3-ol (Example 48, step 3) (1.03 g, 3.21 mmol, 1.00 eq) and methanol (32 mL). Pd(OH)₂/C 20% Het 60-1 (103 mg, 0.06 mmol, 0.015 eq) was added under argon. The reactor was sealed, evacuated and heated to 40° C. before filled with hydrogen gas (reaction pressure of 5 bar was set). The reaction mixture was stirred at 40° C. for 18 hrs. After the reactor was vented carefully, the reaction mixture was filtered (Pall Acrodisc CR 25 mm Syringe Filter 0.45 μm) and rinsed with methanol. The filtrate was concentrated to afford the title compound (556 mg, 92% yield, 80% purity) as a brown oil, which was used without further purification. LCMS: m/z 151.0 [M+H]⁺, ESI pos.

### Step 5: 4-Benzyloxy-6-methyl-2,3-dihydrobenzofuran

To a solution of 6-methyl-2,3-dihydrobenzofuran-4-ol (Example 48, step 4) (393 mg, 2.09 mmol, 1.00 eq, 80% purity) in acetonitrile (4.4 mL) was added potassium carbonate (582 mg, 4.21 mmol, 2.01 eq) followed by benzyl bromide (546 mg, 0.38 mL, 3.20 mmol, 1.53 eq). The reaction mixture was stirred at room temperature for 16 hrs. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 5% ethyl acetate in heptane) to afford the title compound (379 mg, 60% yield, 80% purity) as a colorless oil. LCMS: m/z 241.1 [M+H]⁺, ESI pos.

### Step 6: 4-Benzyloxy-5-bromo-6-methyl-2,3-dihydrobenzofuran

To a solution of 4-benzyloxy-6-methyl-2,3-dihydrobenzofuran (Example 48, step 5) (377 mg, 1.26 mmol, 1.00 eq, 80% purity) in dichloromethane (14 mL) was added portionwise N-bromosuccinimide (294 mg, 1.65 mmol, 1.32 eq) at 0° C. The reaction mixture was stirred at 0° C. for 5.5 hours. The reaction mixture was quenched with saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, gradient 0% to 5% ethyl acetate in heptane). All fractions containing product were combined and concentrated in vacuo. The residue was adsorbed on ISOLUTE HM-N and repurified by flash chromatography (silica gel, gradient 0% to 50% dichloromethane in heptane) to afford the title compound (370 mg, 65% yield, 70% purity) as a colorless oil. LCMS: m/z 319.0; 321.0 (Br isotopes) [M+H]⁺, ESI pos.

### Step 7: 2-(4-Benzyloxy-6-methyl-2,3-dihydrobenzofuran-5-yl)-4,4,5,5-tetramethyl-1,3,2-dioxaborolane

To a solution of 4-benzyloxy-5-bromo-6-methyl-2,3-dihydrobenzofuran (Example 48, step 6) (490 mg, 1.07 mmol, 1.00 eq, 70% purity) and 2-isopropoxy-4,4,5,5-tetramethyl-1,3,2-dioxaborolane (CAS #61676-62-8, 438 mg, 0.48 mL, 2.35 mmol, 2.19 eq) in tetrahydrofuran (6.4 mL) was added dropwise 1.6 M n-butyllithium, solution in hexanes (1.5 mL, 2.40 mmol, 2.23 eq) at −76° C. Let stir at −76° C. for 3 hours. The reaction mixture was warmed to −60° C., quenched with saturated aq. NH₄Cl-solution at −60° C., warmed to room temperature and then extracted with ethyl acetate. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, gradient 0% to 10% ethyl acetate in heptane) to afford the title compound (305 mg, 62% yield, 80% purity) as a colorless oil. LCMS: m/z 367.1 [M+H]⁺, ESI pos.

### Step 8: 6-Methyl-5-(4,4,5,5-tetramethyl-1,3,2-dioxaborolan-2-yl)-2,3-dihydrobenzofuran-4-ol

A solution of 2-(4-benzyloxy-6-methyl-2,3-dihydrobenzofuran-5-yl)-4,4,5,5-tetramethyl-1,3,2-dioxaborolane (Example 48, step 7) (300 mg, 0.66 mmol, 1.00 eq, 80% purity) in ethyl acetate (3.2 mL) and methanol (0.80 mL) was three times alternating evacuated and flushed with argon. Palladium on activated charcoal, 10% Pd basis (30 mg, 0.03 mmol, 0.04 eq) was added. The reaction flask was evacuated, flushed with argon, evacuated and flushed with hydrogen. The reaction mixture was stirred under hydrogen atmosphere (balloon) at room temperature for 2.5 hours. The reaction mixture was filtered and rinsed well with ethyl acetate/methanol. The filtrate was concentrated in vacuo to afford the title compound (225 mg, 99% yield, 80% purity) as a colorless oil, which was used without further purification. LCMS: m/z 277.0 [M+H]⁺, ESI pos.

### Step 9: 5-[2-[[(3R)-1-Ethyl-3-piperidyl]amino]oxazolo[4,5-b]pyridin-5-yl]-6-methyl-2,3-dihydrobenzofuran-4-ol

The title compound was obtained as a light brown solid, LCMS: m/z 395.3 [M+H]⁺, ESI pos, using chemistry similar to that described in Example 19, starting from 5-chloro-N-[(3R)-1-ethyl-3-piperidyl]oxazolo[4,5-b]pyridin-2-amine (Example 14, step 1) and 6-methyl-5-(4,4,5,5-tetramethyl-1,3,2-dioxaborolan-2-yl)-2,3-dihydrobenzofuran-4-ol (Example 48, step 8).

### Example 49

### (3S,5R)-5-[[5-(4-Chloro-2-hydroxy-6-methyl-phenyl)oxazolo[4,5-b]pyridin-2-yl]amino]-1-methyl-piperidin-3-ol

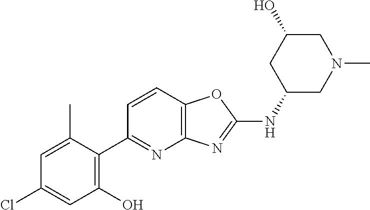 [SMILES: Cc1cc(Cl)cc(O)c1-c1ccc2oc(N[C@@H]3C[C@H](O)CN(C)C3)nc2n1]

### Step 1: tert-Butyl (3R⁵S)-3-[(5-chlorooxazolo[4,5-b]pyridin-2-yl)amino]-5-hydroxy-piperidine-1-carboxylate

To a mixture of 5-chloro-2-(methylthio)oxazolo[4,5-b]pyridine (CAS #1783370-92-2, 200 mg, 0.95 mmol, 1.00 eq) in 1,4-dioxane (1.9 mL) was added tert-butyl (3R,5S)-3-amino-5-hydroxy-piperidine-1-carboxylate (CAS #1932513-59-1, 236 mg, 1.09 mmol, 1.15 eq) followed by triethylamine (112 mg, 0.154 mL, 1.10 mmol, 1.17 eq). The brown solution was stirred at 110° C. for 16 hours. The reaction mixture was cooled to room temperature and extracted with ethyl acetate and saturated NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound (257 mg, 66% yield, 90% purity) as an off-white foam. LCMS: m/z 369.1 [M+H]⁺, ESI pos.

### Step 2: (3S,5R)-5-[(5-Chlorooxazolo[4,5-b]pyridin-2-yl)amino]piperidin-3-ol hydrochloride

To a solution of tert-butyl (3R,5S)-3-[(5-chlorooxazolo[4,5-b]pyridin-2-yl)amino]-5-hydroxy-piperidine-1-carboxylate (Example 49, step 1) (252 mg, 0.61 mmol, 1.00 eq, 90% purity) in dichloromethane (1.2 mL) and methanol (0.60 mL) was added dropwise 4 M HCl in dioxane (1.5 mL, 6.00 mmol, 9.76 eq). The reaction mixture was stirred at room temperature for 2.5 hours. The reaction mixture was concentrated in vacuo to afford the title compound (268 mg, 99% yield, 70% purity) as an off-white foam, which was used without further purification. LCMS: m/z 269.0 [M+H]⁺, ESI pos.

### Step 3: (3S,5R)-5-[(5-Chlorooxazolo[4,5-b]pyridin-2-yl)amino]-1-methyl-piperidin-3-ol

To a mixture of (3S,5R)-5-[(5-chlorooxazolo[4,5-b]pyridin-2-yl)amino]piperidin-3-ol hydrochloride (Example 49, step 2) (264 mg, 0.61 mmol, 1.00 eq, 70% purity) in 1,2-dichloroethane (6.5 mL) was added triethylamine (94 mg, 0.130 mL, 0.93 mmol, 1.54 eq). Let stir for 5 minutes at room temperature. Formaldehyde, 37% aqueous solution (65 mg, 0.060 mL, 0.81 mmol, 1.33 eq) was added followed by sodium triacetoxyborohydride (386 mg, 1.82 mmol, 3.01 eq). The reaction mixture was stirred at room temperature for 1 hour before additional formaldehyde, 37% aqueous solution (65 mg, 0.060 mL, 0.81 mmol, 1.33 eq) was added followed by sodium triacetoxyborohydride (193 mg, 0.91 mmol, 1.50 eq). The reaction mixture was stirred at room temperature for 1 hour. The reaction mixture was extracted with dichloromethane and saturated aq. NaHCO₃-solution. The aqueous layer was backextracted twice with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 20% methanol in dichloromethane) to afford the title compound (146 mg, 81% yield) as a light brown solid. LCMS: m/z 283.1 [M+H]⁺, ESI pos.

### Step 4: (3S,5R)-5-[[5-(4-Chloro-2-hydroxy-6-methyl-phenyl)oxazolo[4,5-b]pyridin-2-yl]amino]-1-methyl-piperidin-3-ol

The title compound was obtained as a light brown powder, LCMS: m/z 389.2 [M+H]⁺, ESI pos, using chemistry similar to that described in Example 19, starting from (3S,5R)-5-[(5-chlorooxazolo[4,5-b]pyridin-2-yl)amino]-1-methyl-piperidin-3-ol (Example 49, step 3) and (4-chloro-2-hydroxy-6-methyl-phenyl)boronic acid (CAS #1207961-50-9).

### Example 50

### 5-Fluoro-3-methyl-2-[2-[[(3R)-1-methyl-3-piperidyl]amino]oxazolo[4,5-b]pyridin-5-yl]phenol

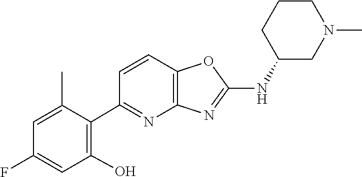 [SMILES: Cc1cc(F)cc(O)c1-c1ccc2oc(N[C@@H]3CCCN(C)C3)nc2n1]

The title compound was obtained as a light brown powder, LCMS: m/z 357.3 [M+H]⁺, ESI pos, using chemistry similar to that described in Example 14, step 2 starting from 5-chloro-N-[(3R)-1-methyl-3-piperidyl]oxazolo[4,5-b]pyridin-2-amine (Example 15, step 1) and (4-fluoro-2-hydroxy-6-methyl-phenyl)boronic acid (Example 45, Intermediate A).

### Example 51

### (3S,5R)-1-Ethyl-5-[[5-[2-hydroxy-6-methyl-4-(trifluoromethyl)phenyl]oxazolo[4,5-b]pyridin-2-yl]amino]piperidin-3-ol

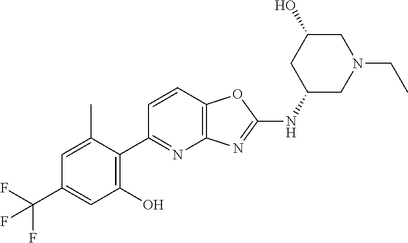 [SMILES: CCN1C[C@@H](O)C[C@@H](Nc2nc3nc(-c4c(C)cc(C(F)(F)F)cc4O)ccc3o2)C1]

The title compound was obtained as a light yellow solid, LCMS: m/z 437.4 [M+H]⁺, ESI pos, using chemistry similar to that described in Example 14, step 2 starting from (3S,5R)-5-[(5-chlorooxazolo[4,5-b]pyridin-2-yl)amino]-1-ethyl-piperidin-3-ol (Example 28, step 3) and 3-methyl-2-(4,4,5,5-tetramethyl-1,3,2-dioxaborolan-2-yl)-5-(trifluoromethyl)phenol (CAS #2557358-38-8).

### Example 52

### 2-[2-[[(3R)-1-(2-Hydroxyethyl)-3-piperidyl]amino]oxazolo[4,5-b]pyridin-5-yl]-3-methyl-5-(trifluoromethyl)phenol

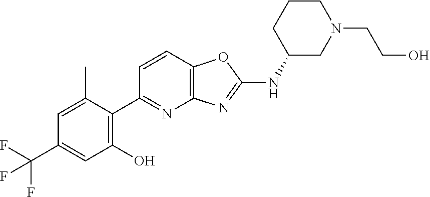 [SMILES: Cc1cc(C(F)(F)F)cc(O)c1-c1ccc2oc(N[C@@H]3CCCN(CCO)C3)nc2n1]

### Step 1: tert-Butyl N-[(3R)-1-(2-hydroxyethyl)-3-piperidyl]carbamate

The title compound was obtained as a light yellow oil, LCMS: m/z 245.1 [M+H]⁺, ESI pos, using chemistry similar to that described in Example 46, step 1 starting from tert-butyl N-[(3R)-3-piperidyl]carbamate (CAS #309956-78-3) and 2-iodoethanol (CAS #624-76-0).

### Step 2: 2-[(3R)-3-[(5-Chlorooxazolo[4,5-b]pyridin-2-yl)amino]-1-piperidyl]ethanol

To a solution of tert-butyl N-[(3R)-1-(2-hydroxyethyl)-3-piperidyl]carbamate (Example 52, step 1) (321 mg, 1.25 mmol, 1.12 eq) in dichloromethane (3.2 mL) and methanol (1.6 mL) was added dropwise 4 M HCl in dioxane (3.6 g, 3.0 mL, 12 mmol, 11.5 eq). Let stir at room temperature for 1 hour. The reaction mixture was concentrated in vacuo. The residue was taken up in 1,4-dioxane (2.0 mL) and 5-chloro-2-(methylthio)oxazolo[4,5-b]pyridine (CAS #1783370-92-2, 220 mg, 1.04 mmol, 1.00 eq) was added followed by triethylamine (421 mg, 0.580 mL, 4.16 mmol, 4.00 eq). The light brown suspension was stirred at 90° C. for 16 hours. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 10% methanol in DCM) to afford the title compound (211 mg, 65% yield) as a light yellow oil. LCMS: m/z 297.1 [M+H]⁺, ESI pos.

### Step 3: 2-[2-[[(3R)-1-(2-Hydroxyethyl)-3-piperidyl]amino]oxazolo[4,5-b]pyridin-5-yl]-3-methyl-5-(trifluoromethyl)phenol

The title compound was obtained as an off-white powder, LCMS: m/z 437.2 [M+H]⁺, ESI pos, using chemistry similar to that described in Example 14, step 2 starting from 2-[(3R)-3-[(5-chlorooxazolo[4,5-b]pyridin-2-yl)amino]-1-piperidyl]ethanol (Example 52, step 2) and 3-methyl-2-(4,4,5,5-tetramethyl-1,3,2-dioxaborolan-2-yl)-5-(trifluoromethyl)phenol (CAS #2557358-38-8).

### Example 53

### 2-[2-[[(3R)-1-Ethyl-3-piperidyl]amino]oxazolo[4,5-b]pyridin-5-yl]-3-fluoro-5-(trifluoromethyl)phenol

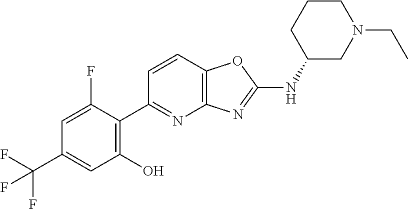 [SMILES: CCN1CCC[C@@H](Nc2nc3nc(-c4c(O)cc(C(F)(F)F)cc4F)ccc3o2)C1]

### Step 1: 2-Bromo-6-fluoro-4-(trifluoromethyl) aniline

To a solution of 2-fluoro-4-trifluoromethylaniline (CAS #69409-98-9, 25.0 g, 140 mmol, 1.00 eq.) in DMF (300 mL) was added NBS (26.1 g, 147 mmol, 1.05 eq.) at −10° C. The mixture was stirred at 25° C. for 12 hrs. To the reaction was added EtOAc (500 mL), washed with brine (500 mL*3), dried over Na₂SO₄, filtered and concentrated in vacuum. The residue was purified by column chromatography (silica gel, Petroleum ether/Ethyl acetate=1/0 to 10/1) to give the title compound (36.0 g, quantitative yield) as yellow oil. LCMS: m/z 257.9 [M+H]⁺, ESI pos.

### Step 2: 2-Fluoro-6-(4,4,5,5-tetramethyl-1,3,2-dioxaborolan-2-yl)-4-(trifluoromethyl) aniline

To a solution of aforementioned 2-bromo-6-fluoro-4-(trifluoromethyl) aniline (30.0 g, 116 mmol, 1.00 eq.) in dioxane (500 mL) was added 4,4,5,5-tetramethyl-2-(4,4,5,5-tetramethyl-1,3,2-dioxaborolan-2-yl)-1,3,2-dioxaborolane (59.1 g, 233 mmol, 2.00 eq.), KOAc (28.5 g, 291 mmol, 2.50 eq.) and Pd(dppf)Cl₂·CH₂Cl₂ (9.50 g, 11.6 mmol, 0.10 eq.) under N₂. The mixture was stirred at 100° C. for 3 hrs. After completion, the reaction was concentrated in vacuum. The residue was added EtOAc (1000 mL), washed with brine (1000 mL), dried over Na₂SO₄, filtered and concentrated in vacuum. The reaction was worked up with additional material to give the title compound (45.0 g, crude) as black oil, which was used next step directly. LCMS: m/z 306.1 [M+H]+, ESI pos.

### Step 3: 2-Amino-3-fluoro-5-(trifluoromethyl)phenol

To a solution of aforementioned 2-fluoro-6-(4,4,5,5-tetramethyl-1,3,2-dioxaborolan-2-yl)-4-(trifluoromethyl) aniline (45.0 g, 148 mmol, 1.00 eq.) in THF (600 mL) was added NaOH (2.00 M, 221 mL, 3.00 eq.) and H₂O₂ (100 g, 885 mmol, 85.0 mL, 30.0% purity, 6.00 eq.) at 0° C. and the reaction was stirred for 3 hrs at 25° C. After completion, to the reaction was added EtOAc (1500 mL), washed with aqueous Na₂SO₃ solution (1500 mL*3), dried over Na₂SO₄, filtered and concentrated under reduced pressure to give a residue. The crude product was purified by reversed-phase HPLC (0.1% FA condition). To give the title compound (11.0 g, 38% yield) as a brown solid. LCMS: m/z 196.0 [M+H]+, ESI pos.

### Step 4: 3-Fluoro-2-iodo-5-(trifluoromethyl)phenol

To a solution of aforementioned 2-amino-3-fluoro-5-(trifluoromethyl)phenol (11.0 g, 56.4 mmol, 1.00 eq.) and H₂SO₄ (40.5 g, 404 mmol, 22.0 mL, 98.0% purity, 7.17 eq.) in H2O (200 mL) and acetone (50.0 mL) was added NaNO₂ (7.78 g, 113 mmol, 2.00 eq.) at 0° C. and the reaction was stirred for 30 min at 0° C. Then CuI (26.8 g, 141 mmol, 2.50 eq) and NaI (21.1 g, 141 mmol, 2.50 eq) was added the reaction at 0° C. and the reaction was stirred for 1.5 hrs at 0° C. After completion, to the reaction was added water (500 mL), washed with EtOAc (300 mL*2). The combined organic layers were washed with brine (300 mL*2), dried over Na₂SO₄, filtered and concentrated under reduced pressure to give a residue. The residue was purified by column chromatography (silica gel, Petroleum ether/Ethyl acetate=1/0 to 10/1) to afford the title compound (20.0 g, crude) as brown oil. ¹H NMR (400 MHz, CDCl₃): δ=7.04 (s, 1H), 6.89 (dd, 1H), 6.76 (s, 1H).

### Step 5: 1-(Ethoxymethoxy)-3-fluoro-2-iodo-5-(trifluoromethyl)benzene

To a solution of aforementioned 3-fluoro-2-iodo-5-(trifluoromethyl)phenol (20.0 g, 65.4 mmol, 1.00 eq.) and chloromethoxyethane (9.09 mL, 98.0 mmol, 1.50 eq.) in DMF (200 mL) was added Cs₂CO₃ (31.9 g, 98.0 mmol, 1.50 eq.) and the mixture was stirred at 25° C. for 2 hrs. After completion, to the reaction was added EtOAc (500 mL), washed with brine (500 mL*3), dried over Na₂SO₄, filtered and concentrated under reduced pressure to give a residue. The residue was purified by column chromatography (silica gel, Petroleum ether/Ethyl acetate=1/0 to 10/1) to afford the title compound (10.0 g, 42% yield) as colourless oil. ¹H NMR (400 MHz, CDCl₃): δ=7.15 (s, 1H), 7.00 (dd, 1H), 5.36 (s, 2H), 3.78 (q, 2H), 1.24 (t, 3H).

### Step 6: 2-[2-(Ethoxymethoxy)-6-fluoro-4-(trifluoromethyl)phenyl]-4,4,5,5-tetramethyl-1,3,2-dioxaborolane

To a solution of aforementioned 1-(ethoxymethoxy)-3-fluoro-2-iodo-5-(trifluoromethyl)benzene (10.0 g, 27.5 mmol, 1.00 eq.) and 2-isopropoxy-4,4,5,5-tetramethyl-1,3,2-dioxaborolane (15.3 g, 82.4 mmol, 16.8 mL, 3.00 eq.) in THF (100 mL) was added n-BuLi (2.50 M, 27.5 mL, 2.50 eq.) at −70° C. and the reaction was stirred for 1 hr at −70° C. After completion, to the reaction was added aqueous NH₄Cl solution (300 mL), stirred for 10 min, extracted with EtOAc (200 mL*2). The combined organic layers were washed with brine (300 mL), dried over Na₂SO₄, filtered and concentrated under reduced pressure to give a residue. The residue was purified by prep-HPLC (Column: Welch Ultimate XB-CN 250*50*10 um; mobile phase: [Hexane-EtOH]; B %: 0%-0%, 7 min) to afford the title compound (7.00 g, 60% yield, 86.3% purity) as a white solid. ¹H NMR (400 MHz, CDCl₃): δ=7.10 (s, 1H), 6.94 (d, 1H), 5.24 (s, 2H), 3.73 (q, 2H), 1.39 (s, 12H), 1.22 (t, 3H).

### Step 7: 5-[2-(Ethoxymethoxy)-6-fluoro-4-(trifluoromethyl)phenyl]-N-[(3R)-1-ethyl-3-piperidyl]oxazolo[4,5-b]pyridin-2-amine

A mixture of 5-chloro-N-[(3R)-1-ethyl-3-piperidyl]oxazolo[4,5-b]pyridin-2-amine (Example 14, step 1) (70 mg, 0.24 mmol, 1.00 eq), aforementioned 2-[2-(ethoxymethoxy)-6-fluoro-4-(trifluoromethyl)phenyl]-4,4,5,5-tetramethyl-1,3,2-dioxaborolane (201 mg, 0.47 mmol, 2.00 eq), potassium carbonate (131 mg, 0.95 mmol, 4.00 eq) and SPhos-Pd-G3 (CAS #1445085-82-4, 25 mg, 0.03 mmol, 0.14 eq) in 1,4-dioxane (2.8 mL) and water (0.70 mL) was flushed with argon and stirred at 110° C. for 1 h. The reaction mixture was cooled to room temperature and extracted with ethyl acetate and water. The aqueous layer was back-extracted with ethyl acetate.

The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in DCM) to afford the title compound (92 mg, 76% yield) as a brown oil. LCMS: m/z 483.3 [M+H]⁺, ESI pos.

### Step 8: 2-[2-[[(3R)-1-Ethyl-3-piperidyl]amino]oxazolo[4,5-b]pyridin-5-yl]-3-fluoro-5-(trifluoromethyl)phenol

To a solution of 5-[2-(Ethoxymethoxy)-6-fluoro-4-(trifluoromethyl)phenyl]-N-[(3R)-1-ethyl-3-piperidyl]oxazolo[4,5-b]pyridin-2-amine (Example 53, step 1) (82 mg, 0.16 mmol, 1.00 eq) in DCM (2.1 mL) was added dropwise trifluoroacetic acid (607 mg, 0.410 mL, 5.32 mmol, 33.0 eq) at 0° C. Let stir at 0° C. for 1.5 hours and at room temperature for 1 hour. The reaction mixture was concentrated in vacuo. The residue was diluted with DCM and extracted with saturated NaHCO₃-solution. The organic layer was washed with water and brine. The organic layers were backextracted twice with DCM. The combined organic layers were dried over sodium sulfate, filtered and concentrated in vacuo. The residue was adsorbed on ISOLUTE HM-N and purified by flash chromatography (silica gel, gradient 0% to 50% (DCM:methanol:NH₄OH 9:1:0.05) in DCM). All fractions containing product were combined and concentrated. The residue was triturated with ethyl acetate/heptane to afford the title compound (38 mg, 53% yield) as an off-white powder. LCMS: m/z 425.2 [M+H]⁺, ESI pos.

### Example 54

### 5-Chloro-2-[2-[[(3R,5S)-1-ethyl-5-fluoro-3-piperidyl]amino]oxazolo[4,5-b]pyridin-5-yl]-3-methyl-phenol

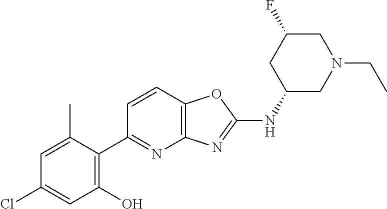 [SMILES: CCN1C[C@@H](F)C[C@@H](Nc2nc3nc(-c4c(C)cc(Cl)cc4O)ccc3o2)C1]

### Step 1: tert-Butyl N-[(3R,5S)-1-ethyl-5-fluoro-3-piperidyl]carbamate

The title compound was obtained as an off-white solid, LCMS: m/z 247.1 [M+H]⁺, ESI pos, using chemistry similar to that described in Example 46, step 1 starting from tert-butyl N-[(3R,5S)-5-fluoro-3-piperidyl]carbamate (CAS #1363378-08-8) and iodoethane (CAS #75-03-6).

### Step 2: (3R,5S)-1-Ethyl-5-fluoro-piperidin-3-amine hydrochloride

The title compound was obtained as an off-white foam, LCMS: m/z 147.1 [M+H]⁺, ESI pos, using chemistry similar to that described in (Example 46, step 2) starting from tert-butyl N-[(3R,5S)-1-ethyl-5-fluoro-3-piperidyl]carbamate (Example 54, step 1).

### Step 3: 5-Chloro-N-[(3R,5S)-1-ethyl-5-fluoro-3-piperidyl]oxazolo[4,5-b]pyridin-2-amine

The title compound was obtained as a yellow oil, LCMS: m/z 299.2 [M+H]⁺, ESI pos, using chemistry similar to that described in Example 46, step 3 starting from 5-chloro-2-(methylthio)oxazolo[4,5-b]pyridine (CAS #1783370-92-2) and (3R,5S)-1-ethyl-5-fluoro-piperidin-3-amine hydrochloride (Example 54, step 2).

### Step 4: 5-Chloro-2-[2-[[(3R,5S)-1-ethyl-5-fluoro-3-piperidyl]amino]oxazolo[4,5-b]pyridin-5-yl]-3-methyl-phenol

The title compound was obtained as an off-white foam, LCMS: m/z 405.2 [M+H]⁺, ESI pos, using chemistry similar to that described in Example 19, starting from 5-chloro-N-[(3R,5S)-1-ethyl-5-fluoro-3-piperidyl]oxazolo[4,5-b]pyridin-2-amine (Example 54, step 3) and (4-chloro-2-hydroxy-6-methyl-phenyl)boronic acid (CAS #1207961-50-9).

### Example 55

### 2-[2-[[(1R,2R)-2-(Dimethylamino)cyclopropyl]amino]oxazolo[4,5-b]pyridin-5-yl]-3-methyl-5-(trifluoromethyl)phenol

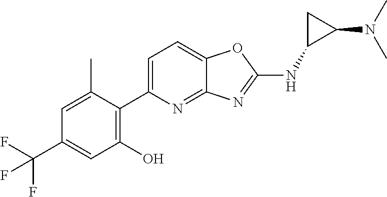 [SMILES: Cc1cc(C(F)(F)F)cc(O)c1-c1ccc2oc(N[C@@H]3C[C@H]3N(C)C)nc2n1]

### Step 1: tert-Butyl N-[(1R,2R)-2-[(5-chlorooxazolo[4,5-b]pyridin-2-yl)amino]cyclopropyl]carbamate

To a mixture of 5-chloro-2-(methylthio)oxazolo[4,5-b]pyridine (CAS #1783370-92-2, 180 mg, 0.85 mmol, 1.00 eq) in 1,4-dioxane (2.0 mL) was added tert-butyl N-[(1R,2R)-2-aminocyclopropyl]carbamate (CAS #1332761-28-0, 155 mg, 0.90 mmol, 1.06 eq) followed by triethylamine (102 mg, 0.14 mL, 1.00 mmol, 1.18 eq). The reaction mixture was stirred at 90° C. for 5 hours and at 100° C. for 16 hours. The reaction mixture was cooled to room temperature and extracted with ethyl acetate and saturated NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 70% ethyl acetate in heptane) to afford the title compound (200 mg, 69% yield) as an off-white foam. LCMS: m/z 325.1 [M+H]⁺, ESI pos.

### Step 2: tert-Butyl N-[(1R,2R)-2-[[5-[2-hydroxy-6-methyl-4-(trifluoromethyl)phenyl]oxazolo[4,5-b]pyridin-2-yl]amino]cyclopropyl]carbamate

The title compound was obtained as a light yellow foam, LCMS: m/z 465.4 [M+H]⁺, ESI pos, using chemistry similar to that described in Example 14, step 2 starting from tert-butyl N-[(1R,2R)-2-[(5-chlorooxazolo[4,5-b]pyridin-2-yl)amino]cyclopropyl]carbamate (Example 55, step 1) and 3-methyl-2-(4,4,5,5-tetramethyl-1,3,2-dioxaborolan-2-yl)-5-(trifluoromethyl)phenol (CAS #2557358-38-8).

### Step 3: 2-[2-[[(1R,2R)-2-Aminocyclopropyl]amino]oxazolo[4,5-b]pyridin-5-yl]-3-methyl-5-(trifluoromethyl)phenol

To a solution of tert-butyl N-[(1R,2R)-2-[[5-[2-hydroxy-6-methyl-4-(trifluoromethyl)phenyl]oxazolo[4,5-b]pyridin-2-yl]amino]cyclopropyl]carbamate (Example 55, step 2) (193 mg, 0.39 mmol, 1.00 eq) in DCM (1.0 mL) and methanol (0.50 mL) was added dropwise 4 M HCl in dioxane (0.88 mL, 3.52 mmol, 8.92 eq) at 0° C. The reaction mixture was stirred at room temperature for 3 hours. The reaction mixture was concentrated and the residue was extracted with DCM/methanol (19:1) and saturated aq. NaHCO₃-solution with some drops of water. The aqueous layer was backextracted twice with DCM/methanol (19:1). The combined organic layers were dried over sodium sulfate, filtered and concentrated in vacuo. The crude product was adsorbed on ISOLUTE HM and purified by flash chromatography (silica gel, gradient 0% to 100% (DCM:methanol:NH₄OH 9:1:0.05) in DCM) to afford the title compound (92 mg, 61% yield) as an off-white solid. LCMS: m/z 365.1 [M+H⁺, ESI pos.

### Step 4: 2-[2-[[(1R,2R)-2-(Dimethylamino)cyclopropyl]amino]oxazolo[4,5-b]pyridin-5-yl]-3-methyl-5-(trifluoromethyl)phenol

To a mixture of 2-[2-[[(1R,2R)-2-aminocyclopropyl]amino]oxazolo[4,5-b]pyridin-5-yl]-3-methyl-5-(trifluoromethyl)phenol (Example 55, step 3) (74 mg, 0.20 mmol, 1.00 eq) in 1,2-dichloroethane (2.0 mL) was added formaldehyde, 37% aqueous solution (33 mg, 0.03 mL, 0.40 mmol, 1.98 eq) followed by portion-wise addition of sodium triacetoxyborohydride (180 mg, 0.85 mmol, 4.18 eq). The reaction mixture was stirred at room temperature for 4 hours. The reaction mixture was extracted with saturated aq. NaHCO₃-solution and three times DCM. The combined organic layers were dried over sodium sulfate, filtered and concentrated in vacuo. The crude product was adsorbed on ISOLUTE HM-N and purified by flash chromatography (silica gel, gradient 0% to 10% methanol in DCM). All fractions containing product were combined and concentrated in vacuo. The residue was adsorbed on ISOLUTE HM-N and repurified by flash chromatography (SI-amine, gradient 0% to 10% methanol in ethyl acetate) to afford the title compound (31 mg, 37% yield) as an off-white solid. LCMS: m/z 393.2 [M+H]⁺, ESI pos.

### Example 56

### 3-Hydroxy-4-[2-[[(3R,5S)-5-hydroxy-1-methyl-3-piperidyl]amino]oxazolo[4,5-b]pyridin-5-yl]-5-methyl-benzonitrile

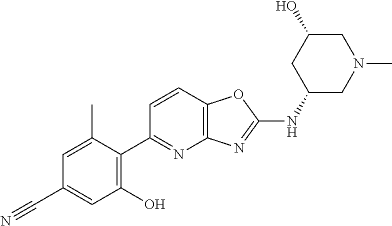 [SMILES: Cc1cc(C#N)cc(O)c1-c1ccc2oc(N[C@@H]3C[C@H](O)CN(C)C3)nc2n1]

The title compound was obtained as an off-white powder, LCMS: m/z 380.2 [M+H]⁺, ESI pos, using chemistry similar to that described in Example 19, starting from (3S,5R)-5-[(5-chlorooxazolo[4,5-b]pyridin-2-yl)amino]-1-methyl-piperidin-3-ol (Example 49, step 3) and (4-cyano-2-hydroxy-6-methyl-phenyl)boronic acid (Example 25, step D).

### Examples 57 and 58

### cis-2-[2-[(3-Hydroxy-3-methyl-cyclobutyl)methylamino]oxazolo[4,5-b]pyridin-5-yl]-3-methyl-5-(trifluoromethyl)phenol and trans-2-[2-[(3-hydroxy-3-methyl-cyclobutyl)methylamino]oxazolo[4,5-b]pyridin-5-yl]-3-methyl-5-(trifluoromethyl)phenol

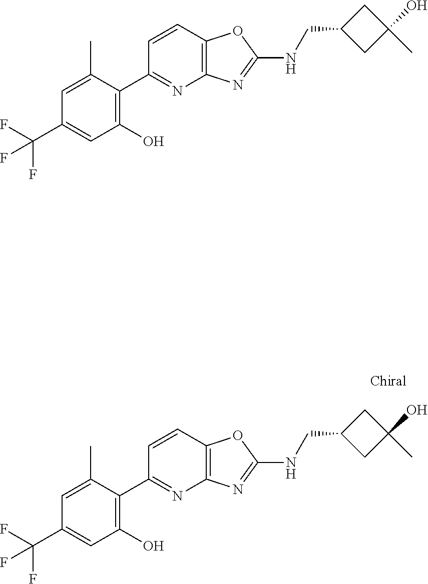 [SMILES: Cc1cc(C(F)(F)F)cc(O)c1-c1ccc2oc(NC[C@H]3C[C@@](C)(O)C3)nc2n1 ; Cc1cc(C(F)(F)F)cc(O)c1-c1ccc2oc(NC[C@H]3C[C@](C)(O)C3)nc2n1]

### Step 1: tert-Butyl N-[(3-hydroxy-3-methyl-cyclobutyl)methyl]carbamate

To a solution of tert-butyl N-[(3-oxocyclobutyl)methyl]carbamate (CAS #130369-09-4, 400 mg, 2.01 mmol, 1.00 eq) in tetrahydrofuran (14 mL) was added dropwise 3.2 M methylmagnesium bromide, solution in 2-methyltetrahydrofuran (CAS #75-16-1, 1.4 mL, 4.48 mmol, 2.23 eq) at 0° C. Let stir at 0° C. for 3 hours before 3.2 M methylmagnesium bromide, solution in 2-methyltetrahydrofuran (CAS #75-16-1, 0.700 mL, 2.24 mmol, 1.12 eq) was added dropwise at 0° C. Let stir at 0° C. for 15 minutes and at room temperature for 15 minutes. The reaction mixture was cooled to 0° C., quenched by dropwise addition of saturated aq. NH4Cl-solution and then extracted with ethyl acetate. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 25 g, gradient 0% to 60% ethyl acetate in heptane) to afford the title compound (334 mg, 70% yield, 90% purity) as a colorless oil. LCMS: m/z 160.1 [M-tBu+H]⁺, ESI pos.

### Step 2: 3-[[(5-Chlorooxazolo[4,5-b]pyridin-2-yl)amino]methyl]-1-methyl-cyclobutanol

To a solution of tert-butyl N-[(3-hydroxy-3-methyl-cyclobutyl)methyl]carbamate (Example 57, step 1) (116 mg, 0.48 mmol, 1.22 eq, 90% purity) in DCM (1.2 mL) and methanol (0.60 mL) was added dropwise 4 M HCl in dioxane (1.39 g, 1.16 mL, 4.63 mmol, 11.6 eq). Let stir at room temperature for 1 hour. The reaction mixture was concentrated in vacuo. The residue was taken up in 1,4-dioxane (0.80 mL) and 5-chloro-2-(methylthio)oxazolo[4,5-b]pyridine (CAS #1783370-92-2, 80 mg, 0.40 mmol, 1.00 eq) was added followed by triethylamine (128 mg, 0.176 mL, 1.26 mmol, 3.17 eq). The brown suspension was stirred at 90° C. for 16 hours. The reaction mixture was cooled to room temperature and extracted with ethyl acetate and saturated aq. NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in DCM) to afford the title compound (94 mg, 79% yield, 90% purity) as an off-white foam. LCMS: m/z 268.3 [M+H]⁺, ESI pos.

### Step 3: cis-2-[2-[(3-Hydroxy-3-methyl-cyclobutyl)methylamino]oxazolo[4,5-b]pyridin-5-yl]-3-methyl-5-(trifluoromethyl)phenol and trans-2-[2-[(3-hydroxy-3-methyl-cyclobutyl)methylamino]oxazolo[4,5-b]pyridin-5-yl]-3-methyl-5-(trifluoromethyl)phenol

A mixture of 3-[[(5-chlorooxazolo[4,5-b]pyridin-2-yl)amino]methyl]-1-methyl-cyclobutanol (Example 57, step 2) (92 mg, 0.31 mmol, 1.00 eq), 3-methyl-2-(4,4,5,5-tetramethyl-1,3,2-dioxaborolan-2-yl)-5-(trifluoromethyl)phenol (CAS #2557358-38-8, 131 mg, 0.43 mmol, 1.40 eq), cesium carbonate (290 mg, 0.89 mmol, 2.88 eq) and XPhos Pd G3 (27 mg, 0.03 mmol, 0.10 eq) in 1,4-dioxane (1.2 mL) and water (0.30 mL) was flushed with argon and stirred at 100° C. for 1 hour and left to cool under stirring in the hot oil bath for 16 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 10% methanol in DCM). All fractions containing product were combined and concentrated in vacuo. The residue was further purified by SFC (column: chiral IC, eluent B: 15% methanol+0.2% diethylamine) to separate the cis/trans mixture to afford cis-2-[2-[(3-hydroxy-3-methyl-cyclobutyl)methylamino]oxazolo[4,5-b]pyridin-5-yl]-3-methyl-5-(trifluoromethyl)phenol (66 mg, 47% yield, 90% purity) as light brown foam (first eluting, Rt=2.11 minutes), LCMS: m/z 408.3 [M+H]⁺, ESI pos and trans-2-[2-[(3-hydroxy-3-methyl-cyclobutyl)methylamino]oxazolo[4,5-b]pyridin-5-yl]-3-methyl-5-(trifluoromethyl)phenol (33 mg, 24% yield, 90% purity) as light brown foam (second eluting, Rt=2.89 minutes), LCMS: m/z 408.3 [M+H]⁺.

### Example 59

### 5-Chloro-2-[2-[(1-ethylazetidin-3-yl)methylamino]oxazolo[4,5-b]pyridin-5-yl]-3-methyl-phenol

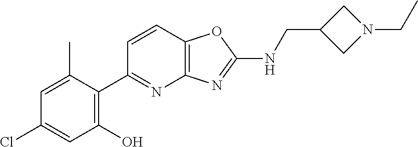 [SMILES: CCN1CC(CNc2nc3nc(-c4c(C)cc(Cl)cc4O)ccc3o2)C1]

### Step A: tert-Butyl 3-[[(5-chlorooxazolo[4,5-b]pyridin-2-yl)amino]methyl]azetidine-1-carboxylate

According to GP1, were 5-chloro-2-(methylthio)oxazolo[4,5-b]pyridine (150 mg, 0.710 mmol), 3-(aminomethyl)azetidine-1-carboxylic acid tert-butyl ester (CAS #325775-44-8, 143 μL, 0.785 mmol), Et₃N (111 μL, 0.796 mmol) stirred in dioxane (1.5 mL) at 90° C. overnight. After workup and purification by flash chromatography (silica gel, EtOAc, heptane 0-70% ethyl acetate), the title compound (168 mg, 70%) was obtained as light yellow solid. LC-MS: m/z 337.1 [M−H]⁻, ESI neg.

### Step B: N-(azetidin-3-ylmethyl)-5-chloro-oxazolo[4,5-b]pyridin-2-amine;2,2,2-trifluoroacetic acid

According to GP3, were aforementioned tert-Butyl 3-[[(5-chlorooxazolo[4,5-b]pyridin-2-yl)amino]methyl]azetidine-1-carboxylate (Example 59, step A) (166 mg, 0.490 mmol) and TFA (340 μL, 4.41 mmol) in DCM (3.58 mL), stirred at room temperature for 30 min to afford the title compound (320 mg, 93%) as light yellow oil. LC-MS: m/z 239.1 [M+H]⁺, ESI pos.

### Step C: 5-Chloro-N-[(1-ethylazetidin-3-yl)methyl]oxazolo[4,5-b]pyridin-2-amine

According to GP4, were aforementioned N-(azetidin-3-ylmethyl)-5-chloro-oxazolo[4,5-b]pyridin-2-amine;2,2,2-trifluoroacetic acid (Example 59, step B) (320 mg, 0.454 mmol), acetaldehyde (46 μL, 0.926 mmol), sodium acetate (74.5 mg, 0.908 mmol), sodium triacetoxyborohydride (144 mg, 0.60 mmol) stirred at 0° C. in DCM (0.965 mL) and methanol (0.965 mL). The reaction mixture was stirred at 0° C. for 30 min and at room temperature for 30 min. After workup, the crude product was adsorbed on silica gel and purified by flash chromatography (TELOS Flash NH₂, 10 g, gradient 0% to 20% methanol in DCM to afford the title compound (97 mg, 7%) as light yellow solid. LC-MS: m/z 267.2 [M+H]⁺, ESI pos.

### Step D: 5-Chloro-2-[2-[(1-ethylazetidin-3-yl)methylamino]oxazolo[4,5-b]pyridin-5-yl]-3-methyl-phenol

According to GP2a, aforementioned (5-chlorooxazolo[4,5-b]pyridin-2-yl)-[(1-ethylazetidin-3-yl)methyl]amine (Example 59, step C) (95 mg, 0.321 mmol), (4-chloro-2-hydroxy-6-methyl-phenyl)boronic acid (CAS #1207961-50-9, 102 mg, 0.549 mmol), potassium carbonate (213 mg, 1.54 mmol) and 1,1′-bis(diphenylphosphino)ferrocene-palladium(ii) dichloride DCM complex (39.8 mg, 0.049 mmol) in 1,4-dioxane (1.99 mL) and water (1 mL) were stirred at 95° C. overnight. After workup, the crude product was adsorbed on ISOLUTE HM-N and first purified by flash chromatography (silica gel, gradient 0% to 10% methanol in DCM) followed by RP HPLC (C₁₈, YMC-Triart, 12 nm, 5 μm, 100×30 mm, ACN/Water+0.1% TEA) affording after lyophilisation the title compound (35 mg, 26%) as off-white solid. LC-MS: m/z 371.1 [M−H]⁻, ESI neg.

### Example 60

### 1-[5-(4-Chloro-2-hydroxy-6-methyl-phenyl)-2-[[rac-(3R)-1-methyl-3-piperidyl]amino]oxazolo[4,5-b]pyridin-7-yl]pyrrolidin-2-one

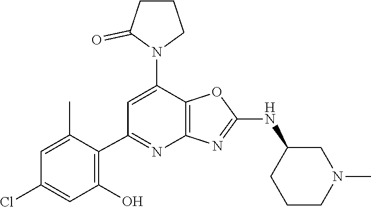 [SMILES: Cc1cc(Cl)cc(O)c1-c1cc(N2CCCC2=O)c2oc(N[C@@H]3CCCN(C)C3)nc2n1]

### Step A: 6-Chloro-4-iodo-2-nitropyridin-3-ol

The apparatus was dried by heating with a heatgun under vacuum. In a three round bottom flask, 6-chloro-4-iodopyridin-3-ol (CAS #877133-58-9, 10.0 g, 39.2 mmol, 1.0 eq) in acetic acid (100 mL) was added fuming nitric acid (6.17 g, 97.9 mmol, 2.5 eq) at 0° C., the mixture was stirred at 25° C. for 12 hrs. After cooling down to ambient temperature, the reaction mixture was quenched ice-water (20 mL), then extracted with EA (30 mL×3). The combined organic layers were washed with brine (30 mL×2), dried over anhydrous sodium sulfate, filtered and the filtrate was concentrated under reduced pressure. The residue was purified by column chromatography (silica gel, petroleum ether:ethyl acetate=2:1 to 1:1) to afford the title compound (2.0 g, 17% yield) as a yellow solid. ¹H NMR (DMSO-d₆, 400 MHz) δ 8.40 (s, 1H).

### Step B: 2-amino-6-chloro-4-iodopyridin-3-ol

To a mixture of aforementioned 6-chloro-4-iodo-2-nitropyridin-3-ol (2.0 g, 6.66 mmol, 1.0 eq) in methanol (30 mL) was added tin(II) chloride (6.31 g, 33.3 mmol, 5.0 eq) and the mixture was stirred at 70° C. for 2 hours. After cooling down to ambient temperature, the reaction mixture was filtered and the filtrate was concentrated under reduce pressure. The residue was purified by silica gel column chromatography (petroleum ether:ethyl acetate=3:1 to 1:1) to afford the title compound (1.80 g, 99% yield) as a yellow solid. LCMS: m/z 270.9 [M+H]⁺, ESI pos.

### Step C: 5-Chloro-7-iodooxazolo[4,5-b]pyridine-2-thiol

The apparatus was dried by heating with a heatgun under vacuum. To a mixture of 2-amino-6-chloro-4-iodopyridin-3-ol (2000.0 mg, 7.39 mmol, 1.0 eq) in DMF (50 mL) was added TCDI (2632.55 mg, 14.79 mmol, 2.0 eq), and the mixture was stirred at 50° C. for 12 hours. After cooling down to ambient temperature, the reaction mixture was quenched by water (20 mL), then extracted with EtOAc (30 mL×3). The combined organic layers were washed with brine (20 mL×2), dried over anhydrous sodium sulfate, filtered and the filtrate was concentrated under reduced pressure to afford the title compound (1000 mg, 43% yield) as a yellow solid. LCMS: m/z 312.8 [M+H]⁺, ESI pos.

### Step D: 2,5-Dichloro-7-iodooxazolo[4,5-b]pyridine and 5-chloro-7-iodo-2-(methylthio) oxazolo[4,5-b]pyridine

To a mixture of 5-chloro-7-iodooxazolo[4,5-b]pyridine-2-thiol (1000.0 mg, 3.2 mmol, 1.0 eq) in oxalyl chloride (20307.5 mg, 160 mmol, 50.0 eq) was added DMF (23.4 mg, 0.32 mmol, 0.1 eq) and the mixture was stirred at 60° C. for 1 hour. After cooling down to ambient temperature, the reaction mixture was concentrated under reduce pressure. The residue was purified by silica gel column chromatography (petroleum ether:ethyl acetate=10:1 to 1:1) to afford 2,5-dichloro-7-iodooxazolo[4,5-b]pyridine (140 mg, 14% yield) as a yellow solid and 5-chloro-7-iodo-2-(methylthio)oxazolo[4,5-b]pyridine (430 mg, 41% yield) as a yellow solid. LCMS: m/z 326.8 [M+H]⁺, ESI pos.

### Step E: (R)-5-Chloro-7-iodo-N-(1-methylpiperidin-3-yl)oxazolo[4,5-b]pyridin-2-amine

To a mixture of (R)-1-methylpiperidin-3-amine (601.5 mg, 5.27 mmol, 4.0 eq) in NMP (5 mL) was added 5-chloro-7-iodo-2-(methylthio)oxazolo[4,5-b]pyridine (430 mg, 1.32 mmol, 1.0 eq) and DIEA (339.7 mg, 2.63 mmol, 2.0 eq), then the mixture was stirred at 25° C. for 2 hrs. The reaction mixture (combined with another batch 0.14 g scale) was quenched by water (1 mL), then diluted with MeOH (3 mL). The mixture was purified by column chromatography (Cis, 0.1% TFA water/ACN condition), then the desired fractions were combined and lyophilized to afford the title compound (550 mg, 106% yield) as a yellow solid. LCMS: m/z 393.0 [M+H]⁺, ESI pos.

### Step F: (R)-1-(5-chloro-2-((1-methylpiperidin-3-yl)amino)oxazolo[4,5-b]pyridin-7-yl)pyrrolidin-2-one

To a mixture of aforementioned (R)-5-chloro-7-iodo-N-(1-methylpiperidin-3-yl)oxazolo[4,5-b]pyridin-2-amine (Example 60, step E) (350 mg, 0.89 mmol, 1.0 eq) and pyrrolidin-2-one (0.54 mL, 7.13 mmol, 8.0 eq) in 1,4-dioxane (5 mL) was added N,N-dimethyl-1,2-ethanediamine (7.86 mg, 0.09 mmol, 0.1 eq), K₂CO₃ (307.55 mg, 2.23 mmol, 2.5 eq) and CuI (16.9 mg, 0.09 mmol, 0.10 eq), and the mixture was stirred at 120° C. for 12 hours under N2. After cooling down to ambient temperature, the mixture was filtered, and the filtrate was concentrated under reduced pressure. The residue was purified by column chromatography (C₁₈, 0.1% TFA in water/MeCN), then the desired fractions were combined and lyophilized to afford the title compound (50.0 mg, 16% yield) as a yellow solid. LCMS: m/z 350.0 [M+H]⁺, ESI pos.

### Step G: 1-[5-(4-Chloro-2-hydroxy-6-methyl-phenyl)-2-[[rac-(3R)-1-methyl-3-piperidyl]amino]oxazolo[4,5-b]pyridin-7-yl]pyrrolidin-2-one

The apparatus was dried by heating with a heatgun under vacuum. To a mixture of aforementioned (R)-1-(5-chloro-2-((1-methylpiperidin-3-yl)amino)oxazolo[4,5-b]pyridin-7-yl)pyrrolidin-2-one (10.0 mg, 0.03 mmol, 1.00 eq), Pd(dppf)Cl₂ (2.09 mg, 0.10 eq) and (4-chloro-2-hydroxy-6-methyl-phenyl)boronic acid (CAS #1207961-50-9, 5.33 mg, 0.03 mmol, 1.00 eq), in 1,4-dioxane (1 mL) and water (0.2 mL) was added CsF (13.03 mg, 0.09 mmol, 3.0 eq) at 25° C., and the mixture was stirred at 120° C. for 2 hours under microwave. After cooling down to ambient temperature, the mixture was filtered, and the filtrate was concentrated under reduced pressure. The residue was purified by column chromatography (C₁₈, 0.1% TFA in water/MeCN), then the desired fractions were combined and lyophilized to afford the title compound (3.8 mg, 23% yield) as a yellow solid. LC-MS (Using SHIMADZU LCMS-2020, Agilent 1200 LC/G1956A MSD and Agilent 1200G6110A, Agilent 1200 LC & Agilent 6110 MSD. Mobile Phase: A: 0.038% TFA in water (v/v); B: 0.019% TFA in Acetonitrile (v/v). Column: Kinetex EVO C18 2.1×30 mm, 5 μm). LCMS: m/z 456.1 [M+H]⁺ ESI pos.

### Example 61

### 2-[2-[[(3R)-1-(3-hydroxycyclobutyl)-3-piperidyl]amino]oxazolo[4,5-b]pyridin-5-yl]-3-methyl-5-(trifluoromethyl)phenol

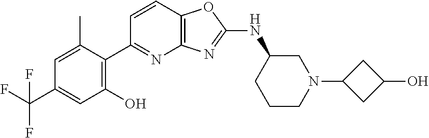 [SMILES: Cc1cc(C(F)(F)F)cc(O)c1-c1ccc2oc(N[C@@H]3CCCN(C4CC(O)C4)C3)nc2n1]

### Step A: tert-butyl (3R)-3-[(5-chlorooxazolo[4,5-b]pyridin-2-yl)amino]piperidine-1-carboxylate

To a solution of 5-chloro-2-(methylthio)oxazolo[4,5-b]pyridine (CAS #1783370-92-2, 0.60 g, 2.84 mmol, 1.00 eq) in 1,4-dioxane (6 mL) was added (3R)-3-aminopiperidine-1-carboxylic acid tert-butyl ester (CAS #188111-79-7, 629 mg, 3.14 mmol, 1.11 eq) followed by triethylamine (444 μL, 3.19 mmol, 1.121 eq). The brown solution was stirred at 90° C. overnight. (3R)-3-aminopiperidine-1-carboxylic acid tert-butyl ester (631.5 mg, 3.15 mmol, 1.110 eq) was added to the reaction mixture and it was stirred for additional two hours and finally over night at 90° C. Further (3R)-3-aminopiperidine-1-carboxylic acid tert-butyl ester (284 mg, 1.42 mmol, 0.50 eq) and triethylamine (222 μL, 1.59 mmol, 0.56 eq) were added to the reaction mixture wehih was stirred for 1 additional hour. Upon reaction completion, the reaction mixture was cooled to room temperature and extracted with ˜60 mL ethyl acetate and ˜15 mL saturated NaHCO₃-solution. The aqueous layer was backextracted with ˜50 mL ethyl acetate. The organic layers were washed with ˜15 mL water and ˜15 mL brine. The combined organic layers were dried over sodium sulfate, filtered and concentrated in vacuo. The crude product was purified by flash chromatography (silica gel, DCM/MeOH 0-10% MeOH) to afford the title compound (1.00 g, 95%) as yellow solid. LCMS: m/z 351.1[M−H]⁻, ESI neg.

### Step B: 5-Chloro-N-[(3R)-3-piperidyl]oxazolo[4,5-b]pyridin-2-amine;hydrochloride

Aforementioned tert-butyl (3R)-3-[(5-chlorooxazolo[4,5-b]pyridin-2-yl)amino]piperidine-1-carboxylate (1.17 g, 3.32 mmol, 1.00 eq) was dissolved in DCM, extra dry (12.2 mL) and methanol (6.07 mL). 4 M HCl 4M in dioxane (8.04 mL, 32.2 mmol, 9.70 eq) was added slowly under ice-bath cooling. After the addition of HCl, the ice bath was removed. The reaction was stirred at room temperature for 3 hrs. The solvent was evaporated afford the title compound as a 1:1 hydrogen chloride which was not further purified (1.09 g, 91% yield, 80% purity) as yellow solid. LCMS: m/z 253.1 [M+H]⁺, ESI pos.

### Step C: 3-[(3R)-3-[(5-Chlorooxazolo[4,5-b]pyridin-2-yl)amino]-1-piperidyl]cyclobutanol

Aforementioned 5-Chloro-N-[(3R)-3-piperidyl]oxazolo[4,5-b]pyridin-2-amine;hydrochloride (100 mg, 346 μmol, 1.00 eq) was suspended in DCM (3 mL) and 3-hydroxycyclobutan-1-one (30.7 mg, 345.8 μmol, 1.00 eq) was added at 0° C. The mixture was stirred for 1.5 h at 0° C. and then allowed to warm to RT. Then sodium triacetoxyborohydride (147 mg, 692 μmol, 2.00 eq) was added followed by acetic acid (39.6 μL, 692 μmol, 2.00 eq). The suspension was stirred at RT overnight. The reaction mixture was diluted with sat. NaHCO₃-solution and extracted two times with EtOAc. The organic layers were washed with water and brine, dried over Na₂SO₄ and concentrated to dryness. The crude product was adsorbed on ISOLUTE HM-N and purified by flash chromatography (silica gel, gradient 0% to 10% MeOH+0.02% NH₄OH in DCM) to the title product (32 mg, 27% yield) as orange viscous oil. LCMS: m/z 323.2; 325.1 (Cl isotopes) [M+H]+, ESI pos.

### Step D: 2-[2-[[(3R)-1-(3-Hydroxycyclobutyl)-3-piperidyl]amino]oxazolo[4,5-b]pyridin-5-yl]-3-methyl-5-(trifluoromethyl)phenol

According to GP2, aforementioned 3-[(3R)-3-[(5-Chlorooxazolo[4,5-b]pyridin-2-yl)amino]-1-piperidyl]cyclobutanol (32 mg, 0.099 mmol, 1.00 eq), 3-methyl-2-(4,4,5,5-tetramethyl-1,3,2-dioxaborolan-2-yl)-5-(trifluoromethyl)phenol (CAS #2557358-38-8, 44.9 mg, 149 μmol, 1.50 eq), cesium carbonate (96.9 mg, 297 μmol, 3.00 eq) and xphos-pd-g3 gt (8.39 mg, 9.91 umol, 0.100 eq) were stirred overnight at 90° C. in 1,4-dioxane (0.5 mL) and water (0.125 mL). After workup, the crude product was adsorbed on ISOLUTE HM-N and purified by flash chromatography (silica gel, gradient 0% to 10% DCM/MeOH+0.02% NH₄OH to afford 117 mg od the title compound but with an unknown impurity so that it was further purified using RP-HPLC (column: YMC-Triart C₁₈, 12 nm, 5 μm, 100×30 mm; 56-50% ACN/water+0.1% HCOOH) to finally afford the title compound (6 mg, 13% yield) as a white solid. LCMS: m/z 461.3 [M+H]⁺, ESI pos.

### Example 62

### 2-[2-(1,3,3a,4,6,6a-Hexahydrofuro[3,4-c]pyrrol-5-yl)oxazolo[4,5-b]pyridin-5-yl]-3-methyl-5-(trifluoromethyl)phenol

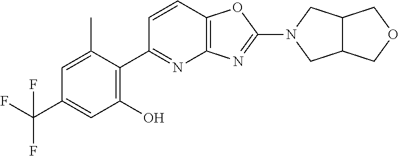 [SMILES: Cc1cc(C(F)(F)F)cc(O)c1-c1ccc2oc(N3CC4COCC4C3)nc2n1]

### Step A: 2-(1,3,3a,4,6,6a-hexahydrofuro[3,4-c]pyrrol-5-yl)-5-chloro-oxazolo[4,5-b]pyridine

According to GP1, 5-chloro-2-(methylthio)oxazolo[4,5-b]pyridine (80 mg, 0.399 mmol), 3,3a,4,5,6,6a-hexahydro-1H-furo[3,4-c]pyrrole (CAS #60889-32-9, 0.441 mmol, 48.5 μL), triethylamine (62.2 μL, 0.447 mmol) were stirred at 90° C. overnight in 1,4-dioxane (0.842 mL). After workup and purification using column chromatography and then HPLC, the title compound was obtained as alight yellow solid (58 mg, 52%). LCMS: m/z 266.1 [M+H]⁺, ESI pos.

### Step B: 2-[2-(1,3,3a,4,6,6a-Hexahydrofuro[3,4-c]pyrrol-5-yl)oxazolo[4,5-b]pyridin-5-yl]-3-methyl-5-(trifluoromethyl)phenol

According to GP2b, were aforementioned 2-[2-[[(3R)-1-(3-Hydroxycyclobutyl)-3-piperidyl]amino]oxazolo[4,5-b]pyridin-5-yl]-3-methyl-5-(trifluoromethyl)phenol (35.0 mg, 0.132 mmol) and [2-hydroxy-6-methyl-4-(trifluoromethyl)phenyl]boronic acid (CAS #2557358-06-0, 42.1 mg, 0.191 mmol) in 1,4-dioxane, extra dry (0.521 mL) and water (0.130 mL), cesium carbonate (123 mg, 0.379 mmol), XPhos Pd G3 (11.5 mg, 0.013 mmol stirred at 100° C. for 1 h. After workup, and purification by flash chromatography (silica gel, EtOAC/heptane: 0-100% EtOAc), the title compound was obtained (32 mg, 57%) as light yellow solid. LCMS: m/z 406.4 [M+H]⁺, ESI pos.

### Example 63

### 5-Chloro-3-methyl-2-[2-[[(3R)-1-(oxetan-3-yl)-3-piperidyl]amino]oxazolo[4,5-b]pyridin-5-yl]phenol

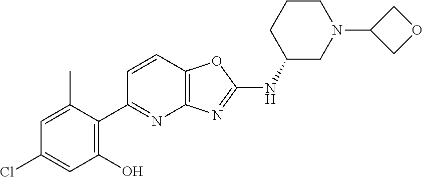 [SMILES: Cc1cc(Cl)cc(O)c1-c1ccc2oc(N[C@@H]3CCCN(C4COC4)C3)nc2n1]

### Step A: 5-Chloro-N-[(3R)-1-(oxetan-3-yl)-3-piperidyl]oxazolo[4,5-b]pyridin-2-amine

According to GP1, 5-chloro-2-(methylthio)oxazolo[4,5-b]pyridine (CAS #1783370-92-2, 300 mg, 1.27 mmol), [(3R)-1-(oxetan-3-yl)-3-piperidyl]amine (CAS #1349700-06-6, 220 mg, 1.41 mmol), triethylamine (213 μL, 1.53 mmol) in 1,4-dioxane (3 mL) was stirred at 90° C. overnight and another 4 hrs at 100° C. A precipitate was formed which was triturated in ether, and then was isolated as a light yellow solid (title compound) (315 mg, 63% yield, 85% purity). LCMS: m/z 309.2 [M+H]⁺, ESI pos.

### Step B: 5-Chloro-3-methyl-2-[2-[[(3R)-1-(oxetan-3-yl)-3-piperidyl]amino]oxazolo[4,5-b]pyridin-5-yl]phenol

According to GP2a, were aforementioned 5-Chloro-N-[(3R)-1-(oxetan-3-yl)-3-piperidyl]oxazolo[4,5-b]pyridin-2-amine (150 mg, 0.413 mmol), (4-chloro-2-hydroxy-6-methyl-phenyl)boronic acid (CAS #1207961-50-9, 131 mg, 0.702 mmol), potassium carbonate (257 mg, 1.86 mmol) and 1,1′-bis(diphenylphosphino)ferrocene-palladium(ii)dichloride DCM complex (50.6 mg, 0.062 mmol) stirred at 100° C. for 16 hrs in 1,4-dioxane (3.4 mL) and water (1.7 mL). After workup and purification (using column and trituration with ether/heptane) the title compound (103 mg, 57%) was obtained as light brown powder. LCMS: m/z 413.3; 415.2 (Cl isotopes) [M−H]⁻, ESI neg.

### Example 64

### 3-Methyl-2-[2-[rac-(4aS,7aS)-3,4a,5,6,7,7a-hexahydro-2H-pyrrolo[3,4-b]1,4]oxazin-4-yl]oxazolo[4,5-b]pyridin-5-yl]-5-(trifluoromethyl)phenol;2,2,2-trifluoroacetic acid and its respective enantiomers 64a and 64b

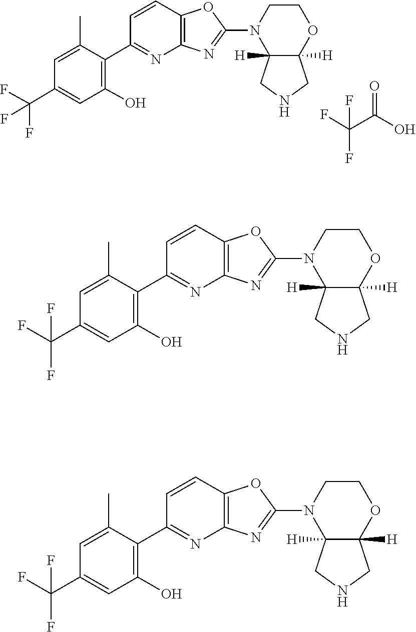

### Step A: tert-butyl rel-(4aS,7aS)-4-(5-chlorooxazolo[4,5-b]pyridin-2-yl)-2,3,4a,5,7,7a-hexahydropyrrolo[3,4-b][1,4]oxazine-6-carboxylate

According to GP1, 5-chloro-2-(methylthio)oxazolo[4,5-b]pyridine (100 mg, 0.498 mmol), tert-butyl rel-(4aS,7aS)-3,4,4a,5,7,7a-hexahydro-2H-pyrrolo[3,4-b][1,4]oxazine-6-carboxylate (CAS #138026-93-4, 125 mg, 0.548 mmol), triethylamine (56.5 mg, 77.8 μL, 0.558 mmol) were stirred at 90° C. over night in 1,4-dioxane (0.61 mL). Additional 4-amino-2-azabicyclo[2.2.2]octane-2-carboxylic acid tert-butyl ester (124 mg, 0.548 mmol) and triethylamine (56.5 mg, 77.8 μL, 0.558 mmol) were added to the reaction mixture and it was stirred for six hours. Then reaction was stopped, after workup and purification the title compound (84 mg, 44%) was obtained as off-white solid. LCMS: m/z 381.1 [M+H]⁺, ESI pos.

### Step B: tert-butyl rel-(4aS,7aS)-4-[5-[2,6-dimethyl-4-(trifluoromethyl)phenyl]oxazolo[4,5-b]pyridin-2-yl]-2,3,4a,5,7,7a-hexahydropyrrolo[3,4-b][1,4]oxazine-6-carboxylate

According to GP2b, aforementioned tert-butyl rel-(4aS,7aS)-4-(5-chlorooxazolo[4,5-b]pyridin-2-yl)-2,3,4a,5,7,7a-hexahydropyrrolo[3,4-b][1,4]oxazine-6-carboxylate (82.0 mg, 0.215 mmol), 3-methyl-2-(4,4,5,5-tetramethyl-1,3,2-dioxaborolan-2-yl)-5-(trifluoromethyl)phenol (94.5 mg, 0.313 mmol), cesium carbonate (202 mg, 0.619 mmol), XPhos Pd G3 (18.8 mg, 0.021 mmol) were stirred at 100° C. in 1,4-dioxane (0.851 mL) and water (0.213 mL) over night. After workup and purification, the title compound (75 mg, 67%) was obtained as a yellow solid. LCMS: m/z 521.3 [M+H]⁺, ESI pos.

### Step C: 3-Methyl-2-[2-[rac-(4aS,7aS)-3,4a,5,6,7,7a-hexahydro-2H-pyrrolo[3,4-b][1,4]oxazin-4-yl]oxazolo[4,5-b]pyridin-5-yl]-5-(trifluoromethyl)phenol;2,2,2-trifluoroacetic acid and its respective enantiomers 64a and 64b

According to GP3, aforementioned tert-butyl rel-(4aS,7aS)-4-[5-[2,6-dimethyl-4-(trifluoromethyl)phenyl]oxazolo[4,5-b]pyridin-2-yl]-2,3,4a,5,7,7a-hexahydropyrrolo[3,4-b][1,4]oxazine-6-carboxylate (75.0 mg, 0.144 mmol), TFA (167 μL, 2.16 mmol) were stirred at room temperature for 1 h in DCM (0.64 mL) to afford the title compound Example 64 (96 mg, 80% purity) as orange solid. LCMS: m/z 419.2 [M−H]⁻, ESI neg. The racemate 64 was then separated using chiral HPLC (column chiral IF, 5 μm, 250×20 mm; 35% MeOH+0.2% DEA, SFC) to afford the two trans enantiomers 64a (20 mg, 25%) and 64b (19 mg, 24% both as orange solids. LCMS: m/z 419.2 [M−H]⁻, ESI neg.

### Example 65

### 2-[2-(1,2,3,5,6,7,8,8a-Octahydroindolizin-6-ylamino)oxazolo[4,5-b]pyridin-5-yl]-5-chloro-3-methyl-phenol

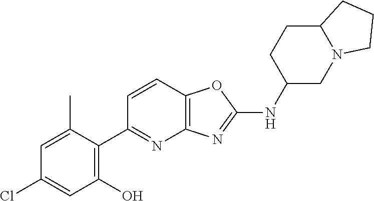 [SMILES: Cc1cc(Cl)cc(O)c1-c1ccc2oc(NC3CCC4CCCN4C3)nc2n1]

### Step A: N-(1,2,3,5,6,7,8,8a-octahydroindolizin-6-yl)-5-chloro-oxazolo[4,5-b]pyridin-2-amine

According to GP1, 5-chloro-2-(methylthio)oxazolo[4,5-b]pyridine (300 mg, 1.5 mmol), indolizidine-6-ylamine (CAS #1824202-77-8, 226 uL, 1.66 mmol), triethylamine (233.4 uL, 1.67 mmol) were stirred at 90° C. for 2 d in 1,4-dioxane (3.16 mL). The reaction was stopped. After workup and purification using column chromatography, the title compound (94 mg, 21%) was obtained as yellow solid. LCMS: m/z 293.1 [M+H]⁺, ESI pos.

### Step B: 2-[2-(1,2,3,5,6,7,8,8a-Octahydroindolizin-6-ylamino)oxazolo[4,5-b]pyridin-5-yl]-5-chloro-3-methyl-phenol

According to GP2a, were aforementioned N-(1,2,3,5,6,7,8,8a-octahydroindolizin-6-yl)-5-chloro-oxazolo[4,5-b]pyridin-2-amine (90 mg, 0.307 mmol), (4-chloro-2-hydroxy-6-methyl-phenyl)boronic acid (CAS #1207961-50-9, 98.1 mg, 0.526 mmol), potassium carbonate (204 mg, 1.48 mmol) and 1,1′-bis(diphenylphosphino)ferrocene-palladium(ii) dichloride DCM complex (38.2 mg, 0.047 mmol) stirred at 95° C. overnight in 1,4-dioxane (1.91 mL) and water (0.954 mL). (4-chloro-2-hydroxy-6-methyl-phenyl)boronic acid (CAS #1207961-50-9, 45.8 mg, 0.246 mmol) and 1,1′-bis(diphenylphosphino)ferrocene-palladium(ii) dichloride DCM complex (19.1 mg, 0.023 mmol) were added to the reaction mixture and it was stirred for two hours at 95° C. After workup and two purifications (one column chromatography and HPLC), the title compound (19 mg, 15%) was obtained as grey solid. LCMS: m/z 397.2 [M+H]⁺, ESI pos.

### Example 66

### 3-Methyl-2-[2-[(1-methylazetidin-2-yl)methylamino]oxazolo[4,5-b]pyridin-5-yl]-5-(trifluoromethyl)phenol and its enantiomers 66a and 66b

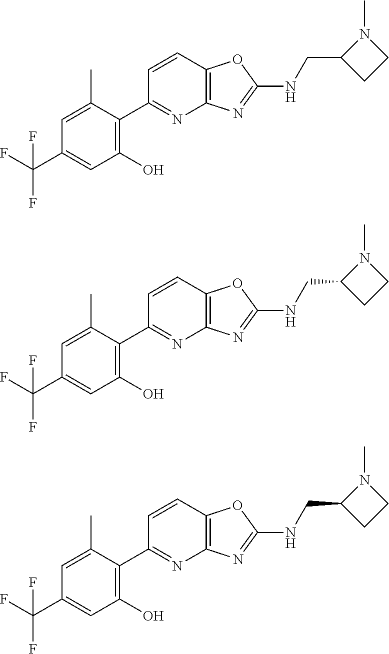 [SMILES: Cc1cc(C(F)(F)F)cc(O)c1-c1ccc2oc(NCC3CCN3C)nc2n1 ; Cc1cc(C(F)(F)F)cc(O)c1-c1ccc2oc(NC[C@H]3CCN3C)nc2n1 ; Cc1cc(C(F)(F)F)cc(O)c1-c1ccc2oc(NC[C@@H]3CCN3C)nc2n1]

### Step A: 5-Chloro-N-[(1-methylazetidin-2-yl)methyl]oxazolo[4,5-b]pyridin-2-amine

According to GP1, 2-aminomethyl-1-methylazetidine (102.5 mg, 0.972 mmol), 2-aminomethyl-1-methylazetidine (102.5 mg, 0.972 mmol), triethylamine (125 μL, 0.897 mmol) were stirred at 90° C. overnight in 1,4-dioxane (1 mL). After workup and purification using column chromatography the title compound (104 mg, 55%) was obtained as off-white solid. LCMS: m/z MS: 253.1; 255.0 [M+H]⁺, ESI pos.

### Step B: 3-Methyl-2-[2-[[(2S)-1-methylazetidin-2-yl]methylamino]oxazolo[4,5-b]pyridin-5-yl]-5-(trifluoromethyl)phenol and its respective Enantiomers 66a and 66b

According to GP2b, aforementioned (5-chlorooxazolo[4,5-b]pyridin-2-yl)-[(1-methylazetidin-2-yl)methyl]amine (104 mg, 0.412 mmol), 3-methyl-2-(4,4,5,5-tetramethyl-1,3,2-dioxaborolan-2-yl)-5-(trifluoromethyl)phenol (196.31 mg, 0.617 mmol), cesium carbonate (402.3 mg, 1.23 mmol) and xphos-pd-G3 (36.6 mg, 0.043 mmol) were stirred at 90° C. overnight in 1,4-dioxane (2 mL) and water (0.50 mL). 3-methyl-2-(4,4,5,5-tetramethyl-1,3,2-dioxaborolan-2-yl)-5-(trifluoromethyl)phenol (98.2 mg, 0.309 mmol), xphos-pd-G3 (17.4 mg, 0.021 mmol) and cesium carbonate (201.14 mg, 0.617 mmol) were added and the mixture was stirred for a additional days at 90° C. After workup and purification the title compound Example 66 (67 mg, 41%) was obtained as white powder. LCMS: m/z 391.3 [M−H]⁻, ESI plus. 33 mg of the title compound Example 66 was purified by chiral HPLC (column chiral IC, 5 μm, 100×4.6 mm; 20-40% MeOH+0.2% DEQ, SFC) to afford two fractions, (13 mg, 34%) and (12 mg, 37%) both as light brown solids.

### Example 67

### 5-Chloro-2-[2-(1H-imidazol-5-ylmethylamino)oxazolo[4,5-b]pyridin-5-yl]-3-methyl-phenol; formic acid

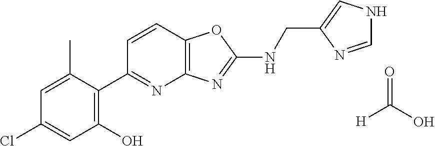

### Step A: (5-Chlorooxazolo[4,5-b]pyridin-2-yl)-(1H-imidazol-5-ylmethyl)amine

To a solution of 5-chloro-2-(methylthio)oxazolo[4,5-b]pyridine (100 mg, 473 μmol, 1.0 eq) in NMP (1 mL) was added 1H-imidazol-5-ylmethylamine;hydrochloride (139 mg, 1.04 mmol, 2.2 eq) followed by triethylamine (178 mg, 245 uL, 1.76 mmol, 3.7 eq). The brown solution was stirred at 130° C. overnight. 1H-imidazol-5-ylmethylamine;hydrochloride (139 mg, 1.04 mmol, 2.2 eq) followed by triethylamine (178 mg, 245 μL, 1.76 mmol, 3.7 eq) was added again to the reaction mixture and stirring was continued at 130° C. overnight. The reaction mixture was cooled to RT and extracted with ethyl acetate (˜40 mL) and saturated NaHCO₃-solution (˜10 mL). The aqueous layer was back extracted with ethyl acetate (˜40 mL). The organic layers were washed with water (˜5 mL) and brine (˜5 mL). The combined organic layers were dried over anhydrous sodium sulfate, filtered and concentrated in vacuo. The crude product was purified by flash chromatography (silica gel, DCM/MeOH 0-20% MeOH), affording the title compound (56 mg, 44% yield) as yellow solid. LCMS m/z: 250.0 [M+H]⁺, ESI pos.

### Step B: 5-Chloro-2-[2-(1H-imidazol-5-ylmethylamino)oxazolo[4,5-b]pyridin-5-yl]-3-methyl-phenol; formic acid

Following to GP2a, (5-chlorooxazolo[4,5-b]pyridin-2-yl)-(1H-imidazol-5-ylmethyl)amine (20 mg, 78.5 μmol, 1.0 eq) and (4-chloro-2-hydroxy-6-methyl-phenyl)boronic acid (CAS #1207961-50-9, 28.7 mg, 153.9 μmol, 1.96 eq) were reacted to afford the title compound (7 mg, 18%) as off-white solid. LCMS m/z: 354.2 [M−H]⁻, ESI neg.

### Example 68

### (3R,5R)-5-[[5-(4-Chloro-2-hydroxy-6-methyl-phenyl)oxazolo[4,5-b]pyridin-2-yl]amino]-1-ethyl-piperidin-3-ol;formic acid

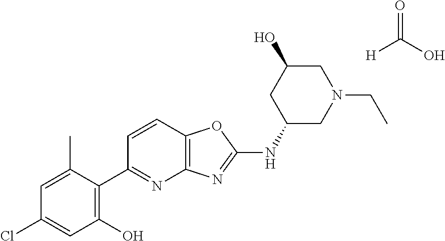

### Step A: (3R,5R)-3-[(5-chlorooxazolo[4,5-b]pyridin-2-yl)aminol-5-hydroxy-piperidine-1-carboxylic acid tert-butyl ester

Following to GP1, 5-chloro-2-(methylthio)oxazolo[4,5-b]pyridine (361 mg, 1.71 mmol, 1.0 eq) and (3R,5R)-3-amino-5-hydroxy-piperidine-1-carboxylic acid tert-butyl ester (443.6 mg, 2.05 mmol, 1.2 eq) were reacted to afford the title compound (690 mg, 99% yield) as light yellow solid. The compound was used without further purification in the next step. LCMS m/z: 369.2; 371.2 [M+H]⁺, ESI pos.

### Step B: (3R,5R)-5-[(5-chlorooxazolo[4,5-b]pyridin-2-yl)amino]piperidin-3-ol; hydrogen chloride

To a solution of (3R,5R)-3-[(5-chlorooxazolo[4,5-b]pyridin-2-yl)amino]-5-hydroxy-piperidine-1-carboxylic acid tert-butyl ester (690 mg, 1.68 mmol, 1.0 eq) in DCM (16 mL) and MeOH (8 mL) was added at rt 4 M HCl in 1,4-dioxane (4.2 mL, 16.8 mmol, 10 eq) dropwise. The reaction mixture was stirred at 23° C. for 16 hrs. The reaction mixture was concentrated in vacuo to give the crude title compound (632 mg, 98% yield) as light brown foam which was used without further purification in the next step. LCMS: m/z 269.1; 271.1 [M+H]⁺, ESI pos.

### Step C: (3R,5R)-5-[(5-chlorooxazolo[4,5-b]pyridin-2-yl)aminol-1-ethyl-piperidin-3-ol

Following to GP4 (3R,5R)-5-[(5-chlorooxazolo[4,5-b]pyridin-2-yl)amino]piperidin-3-ol;hydrochloride (632 mg, 1.66 mmol, 1.0 eq) and acetaldehyde (182.5 mg, 234 μL, 4.14 mmol, 2.5 eq) were reacted to afford the title compound (255 mg, 49%) as light brown foam. LCMS m/z: 297.2; 299.2 [M+H]⁺, ESI pos.

### Step D: (3R,5R)-5-[[5-(4-Chloro-2-hydroxy-6-methyl-phenyl)oxazolo[4,5-b]pyridin-2-yl]amino]-1-ethyl-piperidin-3-ol:formic acid

Following to GP2a, (3R,5R)-5-[(5-chlorooxazolo[4,5-b]pyridin-2-yl)amino]-1-ethyl-piperidin-3-ol (106 mg, 357 μmol, 1.0 eq) and (4-chloro-2-hydroxy-6-methyl-phenyl)boronic acid (CAS #1207961-50-9, 133 mg, 714 μmol, 2.0 eq) were reacted to afford the title compound (18 mg, 11% yield) as white amorph freeze-dried solid. LCMS m/z: 401.2; 403.2 [M−H]⁻, ESI neg.

### Example 69

### 3,6-Dimethyl-2-[2-[[(3R)-1-methyl-3-piperidyl]amino]oxazolo[4,5-b]pyridin-5-ylphenol

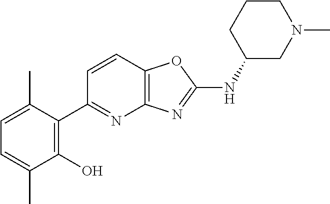 [SMILES: Cc1ccc(C)c(-c2ccc3oc(N[C@@H]4CCCN(C)C4)nc3n2)c1O]

### Step A: [5-(2-Methoxy-3,6-dimethyl-phenyl)oxazolo[4,5-b]pyridin-2-yl]-[(3R)-1-methyl-3-piperidyl]amine

To 5-chloro-N-[(3R)-1-methyl-3-piperidyl]oxazolo[4,5-b]pyridin-2-amine (Example 15, step 1) (41 mg, 153.7 μmol, 1.0 eq) and 2-(2-methoxy-3,6-dimethyl-phenyl)-4,4,5,5-tetramethyl-1,3,2-dioxaborolane (80.6 mg, 307.4 μmol, 2.0 eq) in 1,4-dioxane (2 mL) and water (1 mL) was added under stirring potassium carbonate (95.6 mg, 691.7 μmol, 4.5 eq) at r.t. Afterwards, the yellow reaction solution was flushed with argon for 3 minutes, followed by the addition of 1,1′-bis(diphenylphosphino)ferrocene-palladium(ii)dichloride DCM complex (18.8 mg, 23.06 μmol, 0.15 eq). The dark-reddish-brown reaction mixture was sealed and stirred at 95° C. for 3 hours. The dark-brown/black reaction mixture was cooled to RT and 2-(2-methoxy-3,6-dimethyl-phenyl)-4,4,5,5-tetramethyl-1,3,2-dioxaborolane (80.6 mg, 307.4 μmol, 2.0 eq) was added followed by cesium carbonate (100.2 mg, 307.4 μmol, 2.0 eq) under stirring. Afterwards, the dark-brown/black reaction suspension was flushed with argon for 3 minutes, followed by the addition of XPhos Pd G3 (19.5 mg, 23.1 μmol, 0.15 eq) under stirring at room temperature. The dark-brown/black reaction mixture was flushed again with argon for 3 mins, sealed and stirred at 100° C. for 16 hours. After cooling to r.t., the mixture was extracted with DCM twice (2×50 mL) and sat. NH₄Cl-solution (20 mL). The organic layers were washed with water (20 mL) and brine (20 mL). The aqueous layers were back extracted with DCM (40 mL). The combined organic extracts were dried over anhydrous sodium sulfate, filtered off and concentrated in vacuo. The crude product was purified by flash chromatography (silica gel, 0-10% methanol in DCM) to afford the title compound (26 mg, 46%) as a purple foam. LCMS m/z: 365.3 [M−H]⁻, ESI neg.

### Step B: 3,6-Dimethyl-2-[2-[[(3R)-1-methyl-3-piperidyl]amino]oxazolo[4,5-b]pyridin-5-yl]phenol

To a suspension of [5-(2-methoxy-3,6-dimethyl-phenyl)oxazolo[4,5-b]pyridin-2-yl]-[(3R)-1-methyl-3-piperidyl]amine (26 mg, 70 μmol, 1.0 eq) in DCM (1 mL) was added under stirring 1 M tribromoboron in DCM (936 mg, 360 uL, 360 umol, 5.1 eq) at 0° C. resulting in a brown suspension. The brown reaction mixture was stirred under an atmosphere of argon at room temperature for 3 hours. The light-brown reaction suspension was ice cooled under vigorous stirring, followed by the slow dropwise addition of sat. aq. NaHCO₃-solution (10 mL). Afterwards the reaction mixture was transferred in a separating funnel with 5 mL DCM and was extracted twice with DCM (2×30 mL). The organic phases were washed with water (10 mL) and brine (10 mL). The aqueous phase were back extracted with DCM (20 mL). The combined organic extracts were dried over anhydrous sodium sulfate, filtered off and concentrated in vacuo. The crude was purified by preparative HPLC (column: Gemini NX, 12 nm, 5 μm, 100×30 mm; gradient: MeCN/water+0.10% TEA) to afford the title compound (10 mg, 40% yield) as off-white solid. LCMS m/z: 351.3 [M−H]⁻, ESI neg.

### Example 70

### (3R,5R)-5-[[5-(4-Chloro-2-hydroxy-6-methyl-phenyl)oxazolo[4,5-b]pyridin-2-yl]amino]-1-ethyl-piperidin-3-ol;formic acid

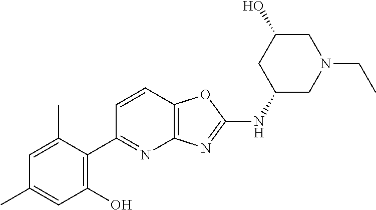 [SMILES: CCN1C[C@@H](O)C[C@@H](Nc2nc3nc(-c4c(C)cc(C)cc4O)ccc3o2)C1]

The title compound was obtained as an white amorph freeze-dried solid, using chemistry similar to that described in Example 28, step 4 starting from (3S,5R)-5-[(5-chlorooxazolo[4,5-b]pyridin-2-yl)amino]-1-ethyl-piperidin-3-ol (Example 28, step 3) and 3,5-dimethyl-2-(4,4,5,5-tetramethyl-1,3,2-dioxaborolan-2-yl)phenol (Example 123, step D). LCMS: m/z 381.4 [M−H]⁻, ESI neg.

### Example 72a and 72b

### 4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile or 4-[2-[(3aR,7aS)-6-ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile and 4-[2-[(3aR,7aS)-6-ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile or 4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile

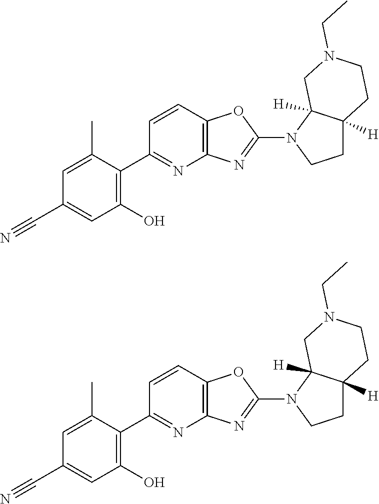 [SMILES: CCN1CC[C@H]2CCN(c3nc4nc(-c5c(C)cc(C#N)cc5O)ccc4o3)[C@H]2C1 ; CCN1CC[C@@H]2CCN(c3nc4nc(-c5c(C)cc(C#N)cc5O)ccc4o3)[C@@H]2C1]

### Step 1: tert-Butyl 1-(5-chlorooxazolo[4,5-b]pyridin-2-yl)-3,3a,4,5,7,7a-hexahydro-2H-pyrrolo[2,3-c]pyridine-6-carboxylate

To a mixture of 5-chloro-2-(methylthio)oxazolo[4,5-b]pyridine (CAS #1783370-92-2, 250 mg, 1.18 mmol, 1.00 eq) in 1,4-dioxane (2.5 mL) was added tert-butyl 1,2,3,3a,4,5,7,7a-octahydropyrrolo[2,3-c]pyridine-6-carboxylate (CAS #1196147-27-9, 308 mg, 1.36 mmol, 1.15 eq) followed by triethylamine (138 mg, 0.190 mL, 1.36 mmol, 1.15 eq). The reaction mixture was stirred at 105° C. for 16 hours and at 115° C. for 2 hours. The reaction mixture was cooled to room temperature and extracted with ethyl acetate and saturated NaHCO₃-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE-HM and purified by flash chromatography (silica gel, 25 g, gradient 0% to 70% ethyl acetate in heptane) to afford the title compound (366 mg, 78% yield) as a light yellow solid. LCMS: m/z 379.2 [M+H]⁺, ESI pos.

### Step 2: 2-(2,3,3a,4,5,6,7,7a-Octahydropyrrolo[2,3-c]pyridin-1-yl)-5-chloro-oxazolo[4,5-b]pyridine hydrochloride

The title compound was obtained as an off-white solid, LCMS: m/z 279.2 [M+H]⁺, ESI pos, using chemistry similar to that described in Example 46, step 2 starting from aforementioned tert-butyl 1-(5-chlorooxazolo[4,5-b]pyridin-2-yl)-3,3a,4,5,7,7a-hexahydro-2H-pyrrolo[2,3-c]pyridine-6-carboxylate (step 1).

### Step 3: 5-Chloro-2-(6-ethyl-3,3a,4,5,7,7a-hexahydro-2H-pyrrolo[2,3-c]pyridin-1-yl)oxazolo[4,5-b]pyridine

To a suspension of aforementioned 2-(2,3,3a,4,5,6,7,7a-Octahydropyrrolo[2,3-c]pyridin-1-yl)-5-chloro-oxazolo[4,5-b]pyridine hydrochloride (step 2) (325 mg, 0.88 mmol, 1.00 eq, 85% purity) in DCM (5.5 mL) was added acetaldehyde (CAS #75-07-0, 94 mg, 0.120 mL, 2.12 mmol, 2.42 eq) followed by sodium acetate (145 mg, 1.77 mmol, 2.02 eq) under ice-bath cooling. Sodium triacetoxyborohydride (280 mg, 1.32 mmol, 1.51 eq) was added in three portions at 0° C. After the addition was complete, the ice-bath was removed and the reaction mixture was stirred at room temperature for 1 hour. The reaction mixture was carefully quenched with saturated aq. NaHCO₃-solution and extracted three times with DCM. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in DCM) to afford the title compound (183 mg, 65% yield) as an orange solid. LCMS: m/z 307.2 [M+H]⁺, ESI pos.

### Step 4: 4-[2-(6-Ethyl-3,3a,4,5,7,7a-hexahydro-2H-pyrrolo[2,3-c]pyridin-1-yl)oxazolo[4,5-b]pyridin-5-yl]-3-hydroxy-5-methyl-benzonitrile

The title compound was obtained as an orange foam, using chemistry similar to that described in Example 14 (step 2) starting from aforementioned 5-chloro-2-(6-ethyl-3,3a,4,5,7,7a-hexahydro-2H-pyrrolo[2,3-c]pyridin-1-yl)oxazolo[4,5-b]pyridine (step 3) and (4-cyano-2-hydroxy-6-methyl-phenyl)boronic acid (Example 25, step D). LCMS: m/z 404.3 [M+H]⁺, ESI pos.

### Step 5: 4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile and 4-[2-[(3aR,7aS)-6-ethyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile

Chiral separation of 4-[2-(6-ethyl-3,3a,4,5,7,7a-hexahydro-2H-pyrrolo[2,3-c]pyridin-1-yl)oxazolo[4,5-b]pyridin-5-yl]-3-hydroxy-5-methyl-benzonitrile (Example 72, step 4) (154 mg, 0.34 mmol, 1.00 eq) by SFC (column: Chiralpak AD, eluent B: 25% methanol+0.2% diethylamine) to afford the two enantiomers example 72a (first eluting, Rt=2.5 minutes) (58 mg, 40% yield) as light brown foam; LCMS: m/z 404.3 [M+H]⁺, ESI pos and example 72b (second eluting, Rt=2.9 minutes) which was repurified by SFC (column: Chiralpak AD, eluent B: 25% methanol+0.2% diethylamine) to afford example 72b (second eluting, Rt=2.9 minutes) (46 mg, 32% yield) as light brown foam; LCMS: m/z 404.3 [M+H]⁺, ESI pos.

### Example 73

### 5-[5-(4-Chloro-2-hydroxy-6-methyl-phenyl)oxazolo[4,5-b]pyridin-2-yl]piperidin-2-one

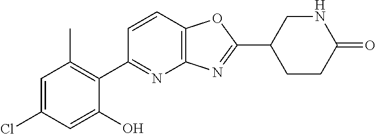 [SMILES: Cc1cc(Cl)cc(O)c1-c1ccc2oc(C3CCC(=O)NC3)nc2n1]

### Step A: 5-(5-Bromooxazolo[4,5-b]pyridin-2-yl)piperidin-2-one

6-Oxopiperidine-3-carboxylic acid (41.0 mg, 0.29 mmol, 1.08 eq) and 2-amino-6-bromopyridin-3-ol (50.0 mg, 0.26 mmol, 1.0 eq) were added to polyphosphoric acid (1.0 mL) and the viscous mixture was heated at 100° C. and stirred for 48 h. The reaction mixture was spooned out and added to a separating funnel containing sat. aq. NaHCO₃ (30 mL) resulting in vigorous bubbling, when bubbling had ceased, DCM (30 mL) was added and shaken. The separated aqueous layer was further extracted with DCM (2×30 mL). The combined organic layers were dried (Na₂SO₄), filtered and concentrated to afford the title compound (38.0 mg, 43% yield) as a light brown solid. LCMS m/z 295.7 (³⁵Cl) [M+H]⁺, ESI pos.

### Step B: 5-[5-(4-Chloro-2-hydroxy-6-methyl-phenyl)oxazolo[4,5-b]pyridin-2-yl]piperidin-2-one

A solution of 5-(5-bromooxazolo[4,5-b]pyridin-2-yl)piperidin-2-one (38.0 mg, 0.13 mmol, 1.0 eq), (4-chloro-2-hydroxy-6-methyl-phenyl)boronic acid (CAS #1207961-50-9, 26.0 mg, 0.14 mmol, 1.09 eq) and potassium carbonate (45.0 mg, 0.33 mmol, 2.54 eq) in water (0.5 mL) and 1,4-dioxane (2 mL) was sparged (bubbling N₂ while sonicating) for 10 min. [1,1′-bis(diphenylphosphino)ferrocene]dichloropalladium(II) (5.0 mg, 0.01 mmol, 0.05 eq) was added and the reaction mixture was heated to 90° C. for 80 min. The crude mixture was concentrated under reduced pressure and loaded onto silica. The crude product was purified by flash chromatography on silica gel (24 g, 0-12% MeOH (with 0.7M NH₃)/EtOAc) to afford the tittle compound (23.0 mg, 48% yield) as a light brown solid. LCMS m/z 358.1 (³⁵Cl) [M+H]+, ESI pos.

### Example 73a and 73b

### (5S)-5-[5-(4-Chloro-2-hydroxy-6-methyl-phenyl)oxazolo[4,5-b]pyridin-2-yl]piperidin-2-one or (5R)-5-[5-(4-Chloro-2-hydroxy-6-methyl-phenyl)oxazolo[4,5-b]pyridin-2-yl]piperidin-2-one and (5R)-5-[5-(4-Chloro-2-hydroxy-6-methyl-phenyl)oxazolo[4,5-b]pyridin-2-yl]piperidin-2-one or (5S)-5-[5-(4-chloro-2-hydroxy-6-methyl-phenyl)oxazolo[4,5-b]pyridin-2-yl]piperidin-2-one

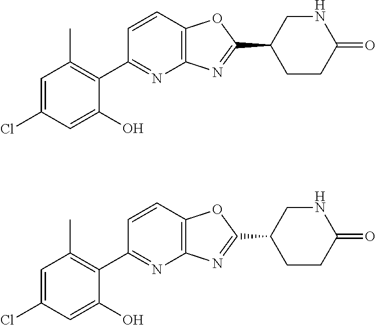 [SMILES: Cc1cc(Cl)cc(O)c1-c1ccc2oc([C@@H]3CCC(=O)NC3)nc2n1 ; Cc1cc(Cl)cc(O)c1-c1ccc2oc([C@H]3CCC(=O)NC3)nc2n1]

The title compounds were obtained after chiral separation of aforementioned 5-[5-(4-Chloro-2-hydroxy-6-methyl-phenyl)oxazolo[4,5-b]pyridin-2-yl]piperidin-2-one (chiral SFC on a Waters prep 15 with UV detection by DAD at 210-400 nm, 40° C., 120 bar; column: Chiralpak IH 10×250 mm, 5 μm; flow rate 15 mL/min at 40% MeOH (0.1% Ammonia), 60% CO₂) to yield 73a (7.1 mg, 15% yield) as an off-white solid (ee 95.2%) and 73b (5.9 mg, 13% yield) was obtained as an-off white solid (ee: 92.6%). LCMS m/z 358.1; 360.1 (Cl isotopes) [M+H]⁺, ESI pos.

### Example 74

### 5-Chloro-2-[2-[[(3R)-1-(3,3-difluoropropyl)-3-piperidyl]amino]oxazolo[4,5-b]pyridin-5-yl]-3-methyl-phenol

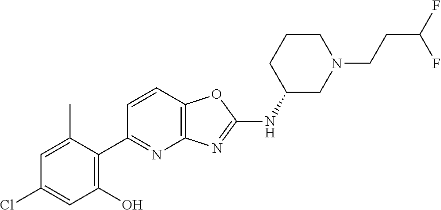 [SMILES: Cc1cc(Cl)cc(O)c1-c1ccc2oc(N[C@@H]3CCCN(CCC(F)F)C3)nc2n1]

### Step 1: 3,3-Difluoropropyl 4-methylbenzenesulfonate

To a solution of 3,3-difluoropropan-1-ol (CAS #461-52-9, 500 mg, 0.452 mL, 5.20 mmol, 1.00 eq) in DCM (15.5 mL) under nitrogen was added triethylamine (1.32 g, 1.8 mL, 13.0 mmol, 2.50 eq) followed by 4-methylbenzenesulfonyl chloride (CAS #98-59-9, 1.19 g, 6.24 mmol, 1.20 eq). The reaction was stirred at room temperature for 3 hours. The reaction mixture was poured into NaHCO₃-solution and extracted three times with DCM. The combined organic layers were washed with brine, dried over sodium sulfate, filtered and concentrated in vacuo. The crude material was adsorbed on silica gel and purified by flash chromatography (silica gel, 20 g, gradient 0% to 30% ethyl acetate in heptane) to afford the title compound (670 mg, 49% yield) as a yellow liquid. LCMS inconclusive, no ionisation.

### Step 2: tert-Butyl (3R)-3-[[5-(4-chloro-2-hydroxy-6-methyl-phenyl)oxazolo[4,5-b]pyridin-2-yl]amino]piperidine-1-carboxylate

The title compound was obtained as a light yellow foam, LCMS: m/z 459.3 [M+H]⁺, ESI pos, using chemistry similar to that described in Example 19, starting from tert-butyl (3R)-3-[(5-chlorooxazolo[4,5-b]pyridin-2-yl)amino]piperidine-1-carboxylate (Example 61, step A) and (4-chloro-2-hydroxy-6-methyl-phenyl)boronic acid (CAS #1207961-50-9).

### Step 3: 5-Chloro-3-methyl-2-[2-[[(3R)-3-piperidyl]amino] oxazolo[4,5-b]pyridin-5-yl]phenol

To a solution of tert-butyl (3R)-3-[[5-(4-chloro-2-hydroxy-6-methyl-phenyl)oxazolo[4,5-b]pyridin-2-yl]amino]piperidine-1-carboxylate (Example 74, step 2) (58 mg, 0.12 mmol, 1.00 eq) in DCM (0.32 mL) and methanol (0.16 mL) was added 4 M HCl, solution in dioxane (0.30 mL, 1.20 mmol, 10.0 eq). The reaction mixture was stirred at room temperature for 2 hours. The reaction mixture was concentrated in vacuo. The residue was dissolved in DCM/methanol (9:1), basified with saturated aq. NaHCO₃-solution and extracted three times with a mixture of DCM/methanol (9:1). The combined organic layers were dried over sodium sulfate, filtered and concentrated in vacuo. The crude product was adsorbed on ISOLUTE HM-N and purified by flash chromatography (Si-amine, gradient 0% to 10% methanol in ethyl acetate) to afford the title compound (25 mg, 55% yield) as an off-white solid. LCMS: m/z 359.2 [M+H]⁺, ESI pos.

### Step 4: 5-Chloro-2-[2-[[(3R)-1-(3,3-difluoropropyl)-3-piperidyl]amino]oxazolo[4,5-b]pyridin-5-yl]-3-methyl-phenol

To a mixture of 5-chloro-3-methyl-2-[2-[[(3R)-3-piperidyl]amino]oxazolo[4,5-b]pyridin-5-yl]phenol (Example 74, step 3) (20 mg, 0.05 mmol, 1.00 eq, 90% purity) in tetrahydrofuran (0.16 mL) and N,N-dimethylformamide (0.16 mL) was added N,N-diisopropylethylamine (19 mg, 0.025 mL, 0.14 mmol, 2.85 eq) followed by dropwise addition of 3,3-difluoropropyl 4-methylbenzenesulfonate (Example 74, step 1) (16 mg, 0.06 mmol, 1.21 eq). The reaction mixture was stirred at 50° C. for 16 hours before 3,3-difluoropropyl 4-methylbenzenesulfonate (8 mg, 0.03 mmol, 0.61 eq) and N,N-dimethylformamide (0.16 mL) were added at room temperature and the reaction mixture was stirred at 50° C. for 16 hours. The reaction mixture was cooled to room temperature and then extracted with ethyl acetate and aq. 10% LiCl-solution. The aqueous layer was back-extracted with ethyl acetate. The organic layers were washed twice with aq. 10% LiCl-solution, once with water and once with brine. The combined organic layers were dried over sodium sulfate, filtered and concentrated in vacuo. The crude product was adsorbed on ISOLUTE HM-N and purified by flash chromatography (silica gel, 4 g, gradient 0% to 100% ethyl acetate in heptane) to afford the title compound (14 mg, 61% yield) as an off-white solid. LCMS: m/z 437.2 [M+H]⁺, ESI pos.

### Example 75

### 5-Chloro-3-methyl-2-[2-[[(3R)-1-[(3-hydroxycyclobutyl)methyl]-3-piperidyl]amino]oxazolo-[4,5-b]pyridin-5-yl]phenol

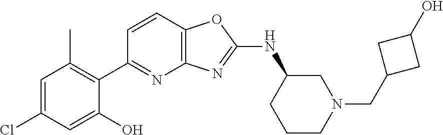 [SMILES: Cc1cc(Cl)cc(O)c1-c1ccc2oc(N[C@@H]3CCCN(CC4CC(O)C4)C3)nc2n1]

### Step A: tert-Butyl (3R)-3-[(5-bromooxazolo[4,5-b]pyridin-2-yl)amino]piperidine-1-carboxylate

(R)-1-Boc-3-aminopiperidine (1.05 g, 5.24 mmol, 1.29 eq) and triethylamine (1.71 mL, 12.24 mmol, 3.0 eq) were added to stirred solution of 5-bromo-2-methylsulfanyl-oxazolo[4,5-b]pyridine (1.0 g, 4.08 mmol, 1.0 eq) in 1,4-dioxane (20 mL) and the reaction mixture was heated to 90° C. for 16 hours. Then additional (R)-1-Boc-3-aminopiperidine (1.05 g, 5.24 mmol, 1.29 eq) and triethylamine (1.71 mL, 12.24 mmol, 3.0 eq) were added to the reaction mixture was heated to 90° C. for a further 18 hours. The reaction mixture was cooled to r.t., diluted with NaHCO₃ (10 mL) and EtOAc (10 mL) and layers were separated. The aqueous layer was extracted with EtOAc (2×10 mL) and the combined organics concentrated in vacuo. The crude product was purified by column chromatography (silica gel, cartridge, 0-100% (EtOAc/isohexane)) to afford the title compound (1.6 g, 86% yield) as a pale brown foam. LCMS m/z 397.2; 399.3 (Br isotopes) [M+H]⁺, ESI pos.

### Step B: 5-Bromo-N-[(3R)-3-piperidyl]oxazolo[4,5-b]pyridin-2-amine;dihydrochloride

To a stirred solution of tert-butyl (3R)-3-[(5-bromooxazolo[4,5-b]pyridin-2-yl)amino]piperidine-1-carboxylate (Example 75, Step A) (2.0 g, 5.03 mmol, 1.0 eq) in 1,4-dioxane (12 mL) at 0° C. was added 4M HCl in dioxane (5.0 mL, 20.0 mmol, 3.97 eq) and the reaction was allowed to warm to r.t. and stirred for 30 mins. Then MeOH (5 mL) was added, and reaction stirred at r.t for a further 1.5 h. The reaction was concentrated under reduced pressure to afford the title compound (1.88 g, 89% yield) as an orange solid. LCMS m/z 297.1; 299.1 (Br isotopes) [M+H]⁺, ESI pos.

### Step C: 3-[(3R)-3-[(5-bromooxazolo[4,5-b]pyridin-2-yl)amino]piperidine-1-carbonyl]-cyclobutanone

To a stirred solution of 5-bromo-N-[(3R)-3-piperidyl]oxazolo[4,5-b]pyridin-2-amine;dihydrochloride (Example 75, step B) (245.0 mg, 0.58 mmol, 1.0 eq) in DCM (5 mL) and N,N-diisopropylethylamine (500 μL, 2.87 mmol, 4.93 eq) was added 3-oxocyclobutanecarboxylic acid (73.0 mg, 0.64 mmol, 1.1 eq) and O-(7-azabenzotriazol-1-yl)-N,N,N′,N′-tetramethyluronium hexafluorophosphate (339.0 mg, 0.89 mmol, 1.53 eq) and the reaction was stirred at rt for 18 hr. The reaction mixture was concentrated under reduced pressure and loaded onto silica. The crude was purified by chromatography on silica (40 g, 0-10% MeOH (0.7 M NH3):EtOAc, to afford the title compound (373.0 mg, 98% yield) as a light brown oil. LCMS m/z 393.2; 395.2 (Br isotopes) [M+H]⁺, ESI pos.

### Step D: 3-[(3R)-3-[[5-(4-chloro-2-hydroxy-6-methyl-phenyl)oxazolo[4,5-b]pyridin-2-yl]amino]piperidine-1-carbonyl]cyclobutanone

A solution of 3-[(3R)-3-[(5-bromooxazolo[4,5-b]pyridin-2-yl)amino]piperidine-1-carbonyl]cyclobutanone (295 mg, 0.56 mmol, 1.0 eq), (4-chloro-2-hydroxy-6-methyl-phenyl)boronic acid (CAS #1207961-50-9, 115 mg, 0.62 mmol, 1.1 eq) and potassium carbonate (196.0 mg, 1.42 mmol, 2.52 eq) in 1,4-Dioxane (5 mL) and water (1 mL) was sparged (bubbling N₂ while sonicating) for 10 min. [1,1′-bis(diphenylphosphino)ferrocene]dichloropalladium(II) (22.0 mg, 0.03 mmol, 0.05 eq) was added and the reaction mixture was heated to 90° C. for 2 h. The crude mixture concentrated under reduced pressure and loaded onto silica. The crude reaction mixture was purified by column chromatography (silica gel, 0-10% MeOH (0.7 M NH₃):EtOAc, to afford title compound (182 mg, 68% yield) as a white solid. LCMS m/z 455.3; 457.2 (Br isotopes) [M+H]⁺, ESI pos.

### Step E: 5-Chloro-3-methyl-2-[2-[[(3R)-1-[(3-hydroxycyclobutyl)methyl]-3-piperidyl]amino]-oxazolo-[4,5-b]pyridin-5-yl]phenol

To a stirred solution of 3-[(3R)-3-[[5-(4-chloro-2-hydroxy-6-methyl-phenyl)oxazolo[4,5-b]pyridin-2-yl]amino]piperidine-1-carbonyl]cyclobutanone (141 mg, 0.31 mmol, 1.0 eq) in THF (9 mL) at 0° C. was added lithium aluminum hydride (2.4 M in THF) (425 μL, 1.02 mmol, 3.29 eq) by dropwise addition. The reaction was stirred for 30 min then allowed to warm to r.t. and stirred for 3.5 hr. The reaction mixture was quenched with MeOH (3 mL) and left to stir overnight. The crude mixture concentrated under reduced pressure and loaded onto silica. The crude reaction mixture was purified by column chromatography (silica gel, 0-20% MeOH (0.7M NH₃):DCM, and then by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130A, 5 μm, 30 mm×100 mm, flow rate 40 mL min-1 eluting with a 0.1% Formic acid in water-MeCN gradient over 12.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 5% MeCN; 0.5-10.5 min, ramped from 5% MeCN to 32.5% MeCN; 10.5-10.6 min, ramped from 32.5% MeCN to 100% MeCN; 10.6-12.5 min, held at 100% MeCN. The clean fractions were evaporated in a Genevac to afford the title compound (43.6 mg, 31% yield) as a white solid. LCMS m/z 433.3; 435.3 (Br isotopes) [M+H]⁺, ESI pos.

### Example 76

### 5-Chloro-2-[2-(1H-imidazol-2-ylmethylamino)oxazolo[4,5-b]pyridin-5-yl]-3-methyl-phenol

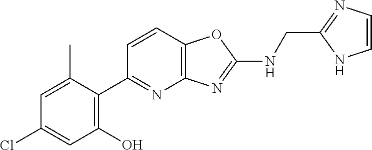 [SMILES: Cc1cc(Cl)cc(O)c1-c1ccc2oc(NCc3ncc[nH]3)nc2n1]

### Step 1: 5-Chloro-N-(1H-imidazol-2-ylmethyl)oxazolo[4,5-b]pyridin-2-amine

To a mixture of 5-chloro-2-(methylthio)oxazolo[4,5-b]pyridine (CAS #1783370-92-2, 150 mg, 0.71 mmol, 1.00 eq) in 1,4-dioxane (1.5 mL) was added 1H-imidazol-2-ylmethylamine dihydrochloride (CAS #53332-80-2, 146 mg, 0.86 mmol, 1.21 eq) followed by triethylamine (290 mg, 0.400 mL, 2.87 mmol, 4.04 eq). The brown suspension was stirred at 90° C. for 16 hours. N-Methyl-2-pyrrolidinone (1.0 mL) was added at room temperature and the reaction mixture was stirred at 140° C. for 3.5 hours. The reaction mixture was cooled to room temperature and extracted with ethyl acetate and 5% aq. LiCl-solution. The aqueous layer was back-extracted with ethyl acetate. The organic layers were washed three times with 5% aq. LiCl-solution, once with water and once with brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (DCM:methanol:NH₄OH 9:1:0.05) in DCM) to afford the title compound (132 mg, 67% yield, 90% purity) as a light yellow solid. LCMS: m/z 250.0 [M+H]⁺, ESI pos.

### Step 2: 5-Chloro-2-[2-(1H-imidazol-2-ylmethylamino)oxazolo[4,5-b]pyridin-5-yl]-3-methyl-phenol

The title compound was obtained as a light brown solid, LCMS: m/z 356.1 [M+H]⁺, ESI pos, using chemistry similar to that described in Example 19, starting from 5-chloro-N-(1H-imidazol-2-ylmethyl)oxazolo[4,5-b]pyridin-2-amine (Example 76, step 1) and (4-chloro-2-hydroxy-6-methyl-phenyl)boronic acid (CAS #1207961-50-9).

### Example 77

### 2-[2-[3-(Hydroxymethyl)-1-piperidyl]oxazolo[4,5-b]pyridin-5-yl]-3-methyl-5-(trifluoromethyl)phenol

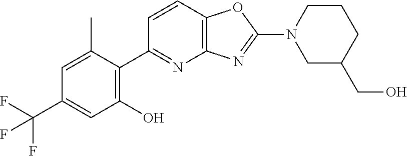 [SMILES: Cc1cc(C(F)(F)F)cc(O)c1-c1ccc2oc(N3CCCC(CO)C3)nc2n1]

### Step 1: [1-(5-Chlorooxazolo[4,5-b]pyridin-2-yl)-3-piperidyl]methanol

A mixture of 5-chloro-2-(methylthio)oxazolo[4,5-b]pyridine (CAS #1783370-92-2, 60 mg, 0.28 mmol, 1.00 eq), 3-piperidylmethanol (CAS #4606-65-9, 36 mg, 0.31 mmol, 1.10 eq) and triethylamine (33 mg, 0.045 mL, 0.32 mmol, 1.14 eq) in 1,4-dioxane (0.60 mL) was stirred at 90° C. for 16 hours. The reaction mixture was then extracted with ethyl acetate and saturated aq. NaHCO₃-solution. The aqueous layer was back-extracted with ethyl acetate. The organic layers were washed with water and brine. The combined organic layers were dried over sodium sulfate, filtered and concentrated in vacuo. The crude product was adsorbed on ISOLUTE HM and purified by flash chromatography (silica gel, 12 g, gradient 0% to 10% methanol in DCM) to the title compound (80 mg, quantitative yield) as a yellow oil. LCMS: m/z 268.1 [M+H]⁺, ESI pos.

### Step 2: 2-[2-[3-(Hydroxymethyl)-1-piperidyl]oxazolo[4,5-b]pyridin-5-yl]-3-methyl-5-(trifluoromethyl)phenol

The title compound was obtained as an off-white foam, LCMS: m/z 408.3 [M+H]⁺, ESI pos, using chemistry similar to that described in Example 14, step 2 starting from [1-(5-chlorooxazolo[4,5-b]pyridin-2-yl)-3-piperidyl]methanol (Example 77, step 1) and 3-methyl-2-(4,4,5,5-tetramethyl-1,3,2-dioxaborolan-2-yl)-5-(trifluoromethyl)phenol (CAS #2557358-38-8).

### Example 78

### 2-[2-[4-(Hydroxymethyl)-1-piperidyl]oxazolo[4,5-b]pyridin-5-yl]-3-methyl-5-(trifluoromethyl)phenol

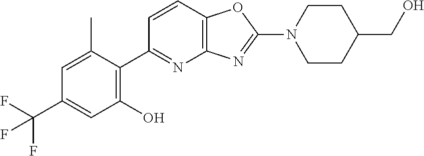 [SMILES: Cc1cc(C(F)(F)F)cc(O)c1-c1ccc2oc(N3CCC(CO)CC3)nc2n1]

### Step 1: [1-(5-Chlorooxazolo[4,5-b]pyridin-2-yl)-4-piperidyl]methanol

A mixture of 5-chloro-2-(methylthio)oxazolo[4,5-b]pyridine (CAS #1783370-92-2, 50 mg, 0.24 mmol, 1.00 eq), 4-piperidylmethanol (CAS #6457-49-4, 30 mg, 0.26 mmol, 1.10 eq) and triethylamine (28 mg, 0.038 mL, 0.27 mmol, 1.15 eq) in 1,4-dioxane (0.50 mL) was stirred at 90° C. for 16 hours. The reaction mixture was cooled to room temperature and then extracted with ethyl acetate and saturated aq. NaHCO₃-solution. The aqueous layer was back-extracted with ethyl acetate. The organic layers were washed with water and brine. The combined organic layers were dried over sodium sulfate, filtered and concentrated in vacuo. The crude product was adsorbed on ISOLUTE HM and purified by flash chromatography (silica gel, 12 g, gradient 0% to 10% methanol in DCM) to afford the title compound (65 mg, 97% yield) as an off-white solid. LCMS: m/z 268.3 [M+H]⁺, ESI pos.

### Step 2: [1-[5-[2-Methoxy-6-methyl-4-(trifluoromethyl)phenyl]oxazolo[4,5-b]pyridin-2-yl]-4-piperidyl]methanol

The title compound was obtained as a light yellow waxy solid, using chemistry similar to that described in Example 19, starting from [1-(5-chlorooxazolo[4,5-b]pyridin-2-yl)-4-piperidyl]methanol (Example 78, step 1) and 2-[2-methoxy-6-methyl-4-(trifluoromethyl)phenyl]-4,4,5,5-tetramethyl-1,3,2-dioxaborolane (CAS #2557358-25-3). LCMS: m/z 422.3 [M+H]⁺, ESI pos.

### Step 3: 2-[2-[4-(Hydroxymethyl)-1-piperidyl]oxazolo[4,5-b]pyridin-5-yl]-3-methyl-5-(trifluoromethyl)phenol

The title compound was obtained as a white solid, using chemistry similar to that described in Example 3 (step 3) starting from [1-[5-[2-methoxy-6-methyl-4-(trifluoromethyl)phenyl]oxazolo[4,5-b]pyridin-2-yl]-4-piperidyl]methanol (Example 78, step 2) and boron tribromide (1 M solution in dichloromethane). LCMS: m/z 408.3 [M+H]⁺, ESI pos.

### Example 79

### 2-[2-(1-Ethyl-1,7-diazaspiro[3.5]nonan-7-yl)oxazolo[4,5-b]pyridin-5-yl]-3-methyl-5-(trifluoromethyl)phenol

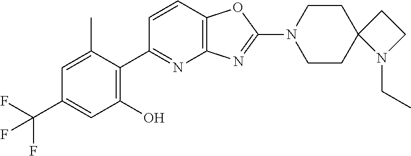 [SMILES: CCN1CCC12CCN(c1nc3nc(-c4c(C)cc(C(F)(F)F)cc4O)ccc3o1)CC2]

### Step 1: 5-Chloro-2-(1-ethyl-1,7-diazaspiro[3.5]nonan-7-yl)oxazolo[4,5-b]pyridine

To a mixture of 5-chloro-2-(methylthio)oxazolo[4,5-b]pyridine (CAS #1783370-92-2, 80 mg, 0.38 mmol, 1.00 eq) in 1,4-dioxane (0.80 mL) was added 1-ethyl-1,7-diazaspiro[3.5]nonane (CAS #1422139-09-0, 66 mg, 0.43 mmol, 1.13 eq) followed by triethylamine (44 mg, 0.060 mL, 0.43 mmol, 1.14 eq). The brown solution was stirred at 90° C. for 16 hours. The reaction mixture was cooled to room temperature and extracted with ethyl acetate and saturated aq. NaHCO₃-solution. The aqueous layer was back-extracted with ethyl acetate. The organic layers were washed with water and brine. The combined organic layers were dried over sodium sulfate, filtered and concentrated in vacuo. The crude product was adsorbed on ISOLUTE HM and purified by flash chromatography (silica gel, gradient 0% to 100% (DCM:methanol:NH₄OH 9:1:0.05) in DCM) to afford the title compound (106 mg, 87% yield) as a light yellow solid. LCMS: m/z 307.2 [M+H]⁺, ESI pos.

### Step 2: 2-[2-(1-Ethyl-1,7-diazaspiro[3.5]nonan-7-yl)oxazolo[4,5-b]pyridin-5-yl]-3-methyl-5-(trifluoromethyl)phenol

The title compound was obtained as an off-white foam, LCMS: m/z 447.3 [M+H]⁺, ESI pos, using chemistry similar to that described in Example 14, step 2 starting from 5-chloro-2-(1-ethyl-1,7-diazaspiro[3.5]nonan-7-yl)oxazolo[4,5-b]pyridine (Example 79, step 1) and 3-methyl-2-(4,4,5,5-tetramethyl-1,3,2-dioxaborolan-2-yl)-5-(trifluoromethyl)phenol (CAS #2557358-38-8).

### Example 80

### 3-Hydroxy-5-methyl-4-[2-[rel-(4aS,7aR)-6-ethyl-2,3,4a,5,7,7a-hexahydropyrrolo[3,4-b][1,4]oxazin-4-yloxazolo[4,5-b]pyridin-5-ylbenzonitrile

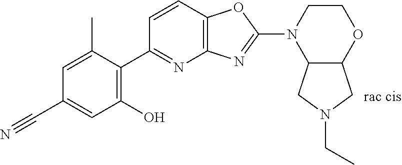 [SMILES: CCN1CC2OCCN(c3nc4nc(-c5c(C)cc(C#N)cc5O)ccc4o3)C2C1]

### Step A: tert-Butyl rel-(4aS,7aR)-6-ethyl-2,3,4a,5,7,7a-hexahydropyrrolo[3,4-b][1,4]oxazine-4-carboxylate

In a sealed tube, to a mixture of commercially available tert-butyl rel-(4aS,7aR)-3,4a,5,6,7,7a-hexahydro-2H-pyrrolo[3,4-b][1,4]oxazine-4-carboxylate (CAS #1312131-49-9, 150 mg, 657 μmol, 1.0 eq,) in tetrahydrofuran (3.1 mL) was added N,N-diisopropylethylamine (212 mg, 287 μL, 1.6 mmol, 2.5 eq) followed by dropwise addition of ethyl iodide (123 mg, 63.7 μL, 788 μmol, 1.2 eq) at r.t. Then, the mixture was stirred at 40° C. for 6 h30, then at r.t. overnight. The reaction mixture was extracted with ethyl acetate, sodium bicarbonate and water. The aqueous layers were back extracted with ethyl acetate twice. The combined organic layers were washed with brine and water, then dried over anhydrous sodium sulfate, filtered and concentrated in vacuo. The crude title compound (149 mg, 84% yield) was obtained as a yellow viscous oil, that was used in the next step without purification. LCMS m/z: 257.2 [M+H]⁺, ESI pos.

### Step B: rel-(4aS,7aR)-6-Ethyl-3,4,4a,5,7,7a-hexahydro-2H-pyrrolo[3,4-b][1,4]oxazine;hydrogen chloride

To a solution of tert-butyl rel-(4aS,7aR)-6-ethyl-2,3,4a,5,7,7a-hexahydropyrrolo[3,4-b][1,4]oxazine-4-carboxylate (147 mg, 544 μmol, 1.0 eq) in dichloromethane (4.2 mL) and methanol (2.1 mL) was added dropwise 4 M HCl in dioxane (1.77 g, 1.36 mL, 5.44 mmol, 10 eq). The reaction mixture was stirred at r.t. overnight under an atmosphere of argon. The solvents were evaporated in vacuo. The title compound (154 mg, 110% yield) was obtained as a light yellow solid, that was used in the next step without purification. LCMS m/z: 157.1 [M+H]⁺, ESI pos.

### Step C: 5-Chloro-2-[rel-(4aS,7aR)-6-ethyl-2,3,4a,5,7,7a-hexahydropyrrolo[3,4-b][1,4]oxazin-4-yl]oxazolo[4,5-b]pyridine

Following to GP1, 5-chloro-2-(methylthio)oxazolo[4,5-b]pyridine (120 mg, 586 μmol, 1.0 eq) and rel-(4aS,7aR)-6-Ethyl-3,4,4a,5,7,7a-hexahydro-2H-pyrrolo[3,4-b][1,4]oxazine;hydrogen chloride (150.6 mg, 703.31 μmol, 1.2 eq) were reacted at 120° C. overnight. After addition of NMP (0.5 mL) stirring was continued at 150° C. for 14 hrs. After work-up, the crude was purified by flash chromatography (gradient 0-80% DCM:MeOH:NH₄OH (110:10:1) in DCM) to afford the title compound (69.5 mg, 37% yield) as brown solid. LCMS: 309.2 [M+H]⁺, ESI pos.

### Step D: 3-Hydroxy-5-methyl-4-[2-[rel-(4aS,7aR)-6-ethyl-2,3,4a,5,7,7a-hexahydropyrrolo[3,4-b][1,4]oxazin-4-yl]oxazolo[4,5-b]pyridin-5-yl]benzonitrile

Following to GP2b, 5-chloro-2-[rel-(4aS,7aR)-6-ethyl-2,3,4a,5,7,7a-hexahydropyrrolo[3,4-b][1,4]oxazin-4-yl]oxazolo[4,5-b]pyridine (68 mg, 220 μmol, 1.0 eq) and (4-cyano-2-hydroxy-6-methyl-phenyl)boronic acid (Example 25, step D) (66.3 mg, 374 μmol, 1.7 eq) were reacted. The pure product was obtained after HPLC chromatography (column: YMC-Triart C₁₈, 12 nm, 5 μm, 100×30 mm; gradient: 15-35-50-100 MeCN in water; 11 mins; MeCN/(water+0.1% TEA)) as a white solid (20.7 mg, 22% yield). LCMS: 406.3 [M+H]⁺, ESI pos.

### Example 81

### 3-Hydroxy-5-methyl-4-[2-(7-methyl-2,3,4,4a,5,6,8,8a-octahydro-1,7-naphthyridin-1-yl)oxazolo[4,5-b]pyridin-5-yl]benzonitrile

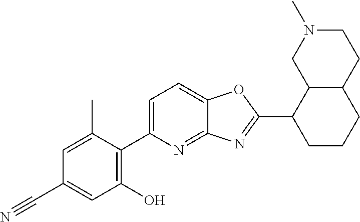 [SMILES: Cc1cc(C#N)cc(O)c1-c1ccc2oc(C3CCCC4CCN(C)CC43)nc2n1]

### Step A: 5-Chloro-2-(7-methyl-2,3,4,4a,5,6,8,8a-octahydro-1,7-naphthyridin-1-yl)oxazolo[4,5-b]pyridine

Following to GP1 5-chloro-2-(methylthio)oxazolo[4,5-b]pyridine (225 mg, 1.1 mmol, 1.0 eq) and 7-methyl-2,3,4,4a,5,6,8,8a-octahydro-1H-1,7-naphthyridine;dihydrochloride (300 mg, 1.32 mmol, 1.2 eq) were reacted in a sealed tube at 120° C. for 2.5 hours. Afterwards, NMP (3 mL) was added and the reaction was stirred at 140° C. overnight. After work-up and purification by flash chromatography (gradient 0-10% MeOH in DCM) the title compound was obtained (36.8 mg, 10% yield) as light brown viscous oil. LCMS: 307.2 [M+H]⁺, ESI pos.

### Step B: 3-Hydroxy-5-methyl-4-[2-(7-methyl-2,3,4,4a,5,6,8,8a-octahydro-1,7-naphthyridin-1-yl)oxazolo[4,5-b]pyridin-5-yl]benzonitrile

Following to GP2b 5-chloro-2-(7-methyl-2,3,4,4a,5,6,8,8a-octahydro-1,7-naphthyridin-1-yl)oxazolo[4,5-b]pyridine (35 mg, 108 μmol, 1.0 eq) and (4-cyano-2-hydroxy-6-methyl-phenyl)boronic acid (Example 25, step D) were reacted to yield the title compound (5.8 mg, 13% yield) as a white solid. LCMS: 402.3 [M−H]⁻, ESI neg.

### Example 82

### 2-Hydroxy-4-methyl-3-[2-[[(3R)-1-methyl-3-piperidyl]amino]oxazolo[4,5-b]pyridin-5-yl]benzonitrile;2,2,2-trifluoroacetic acid

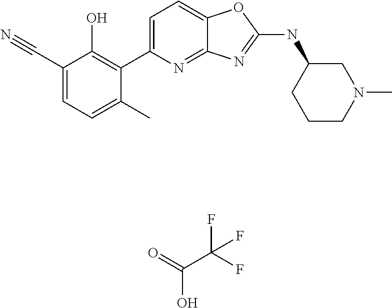 [SMILES: Cc1ccc(C#N)c(O)c1-c1ccc2oc(N[C@@H]3CCCN(C)C3)nc2n1 ; O=C(O)C(F)(F)F]

### Step A: 3-Bromo-2-hydroxy-4-methylbenzonitrile

To a mixture of diisopropylamine (3.03 g, 30.0 mmol, 1.0 eq) and NBS (6.42 g, 36.05 mmol, 1.2 eq) in toluene (60 mL) was added 2-hydroxy-4-methylbenzonitrile (4.0 g, 30.04 mmol, 1.0 eq) at 0° C., then stirred at 20° C. for 12 hours. The above reaction solution was diluted with water (200 mL), extracted with ethyl acetate (50 mL*3). The combined organic phases were washed with brine (100 mL), dried over anhydrous sodium sulfate, filtered and the filtrate was concentrated under reduced pressure. The residue was purified by column chromatography (silica gel, petroleum ether:ethyl acetate=1:0 to 2:1) to afford the title compound (1.10 g, 17% yield) as a yellow solid. ¹H NMR (CDCl₃, 400 MHz) δ [ppm]: 7.42 (d, 1H), 6.91 (d, 1H), 6.23 (br. s, 1H), 2.48 (s, 3H).

### Step B: 3-Bromo-4-methyl-2-((2-(trimethylsilyl)ethoxy)methoxy)benzonitrile

To a solution of 3-bromo-2-hydroxy-4-methylbenzonitrile (1.10 g, 5.19 mmol, 1.0 eq) in MeCN (10 mL) was added K₂CO₃ (1.43 g, 10.4 mmol, 2.0 eq), then 2-(trimethylsilyl)ethoxymethyl chloride (1.38 mL, 7.78 mmol, 1.5 eq) was added, stirred at 20° C. for 1 hour. The above reaction solution was diluted with water (50 mL), extracted with ethyl acetate (20 mL*3). The combined organic phases were washed with brine (50 mL), dried over anhydrous sodium sulfate, filtered and the filtrate was concentrated under reduced pressure. The residue was purified by silica gel column chromatography (petroleum ether:ethyl acetate=1:0 to 10:1) to afford the title compound (1.10 g, 62% yield) as a yellow oil. ¹H NMR (CDCl₃, 400 MHz) δ [ppm]: 7.44 (d, 1H), 7.10 (d, 1H), 5.34 (s, 2H), 4.09-3.96 (m, 2H), 2.49 (s, 3H), 1.08-1.01 (m, 2H), 0.05 (s, 9H).

### Step C: 4-Methyl-3-(4,4,5,5-tetramethyl-1,3,2-dioxaborolan-2-yl)-2-((2-(trimethylsilyl) ethoxy)methoxy)benzonitrile

To a solution of 3-bromo-4-methyl-2-((2-(trimethylsilyl)ethoxy)methoxy)benzonitrile (1.0 g, 2.92 mmol, 1.0 eq) and 2-isopropoxy-4,4,5,5-tetramethyl-1,3,2-dioxaborolane (1.09 g, 5.84 mmol, 2.0 eq) in THF (20 mL) was added iPrMgCl-LiCl (4.49 mL, 1.3 M solution in hexane, 2.0 eq) under nitrogen atmosphere, at 0° C., and then stirred for 2 hours at 20° C. The above reaction solution was quenched with sodium sulfite (150 mL), extracted with ethyl acetate (50 mL*3). The combined organic phases were washed with brine (100 mL), dried over anhydrous sodium sulfate, filtered and the filtrate was concentrated under reduced pressure to give a yellow oil. The residue was purified by silica gel column chromatography (petroleum ether:ethyl acetate=1:0 to 10:1) to afford the title compound (0.30 g, 26% yield) as a yellow oil. ¹H NMR (CDCl₃, 400 MHz) δ [ppm]: 7.50 (d, 1H), 6.95 (d, 1H), 5.24 (s, 2H), 4.03-3.88 (m, 2H), 2.45 (s, 3H), 1.40 (s, 12H), 1.08-0.97 (m, 2H), 0 (s, 9H).

### Step D: (R)-4-Methyl-3-(2-((1-methylpiperidin-3-yl)amino)oxazolo[4,5-b]pyridin-5-yl)-2-((2-(trimethylsilyl)ethoxy)methoxy)benzonitrile

To a solution of 4-methyl-3-(4,4,5,5-tetramethyl-1,3,2-dioxaborolan-2-yl)-2-(2-trimethylsilylethoxymethoxy)benzonitrile (0.080 g, 0.21 mmol, 1.0 eq) in 1,4-dioxane (1 mL) and water (0.2 mL) was added (R)-5-bromo-N-(1-methylpiperidin-3-yl)oxazolo[4,5-b]pyridin-2-amine (0.080 g, 0.19 mmol, 0.92 eq), CsF (0.078 g, 0.51 mmol, 2.5 eq) and XPhosPdG3 (0.035 g, 0.04 mmol, 0.2 eq), then the above reaction mixture was stirred at 95° C. for 1 hour. The above reaction solution was diluted with water (100 mL) and extracted with ethyl acetate (20 mL*3). The combined organic phases were washed with brine (50 mL), dried over anhydrous sodium sulfate, filtered and the filtrate was concentrated under reduced pressure, then purified by prep-TLC (DCM:MeOH=10:1, Rf=0.1) to afford the title compound (0.040 g, 38% yield) as a yellow solid. LCMS: m/z 494.2 [M+H]⁺, ESI pos. ¹H NMR (DMSO-d₆, 400 MHz) δ=7.57-7.45 (m, 2H), 7.10 (d, 1H), 6.95 (d, 1H), 6.62-6.18 (m, 1H), 4.81 (s, 2H), 4.30-4.18 (m, 1H), 3.61-3.40 (m, 2H), 2.81-2.47 (m, 3H), 2.33 (s, 3H), 2.21 (s, 3H), 2.11 (s, 1H), 1.96-1.81 (m, 2H), 1.72-1.59 (m, 2H), 0.90-0.76 (m, 2H), 0 (s, 9H).

### Step E: 2-Hydroxy-4-methyl-3-[2-[[(3R)-1-methyl-3-piperidyl]amino]oxazolo[4,5-b]pyridin-5-yl]benzonitrile;2,2,2-trifluoroacetic acid

To a solution of (R)-4-methyl-3-(2-((1-methylpiperidin-3-yl)amino)oxazolo[4,5-b]pyridin-5-yl)-2-((2-(trimethylsilyl)ethoxy)methoxy)benzonitrile (20 mg, 0.04 mmol, 1.0 eq) in DCM (2 mL) was added TFA (1.0 mL) then the above reaction mixture was stirred at 20° C. for 1 h. The above reaction mixture was concentrated under reduced pressure. The crude product was purified by prep-HPLC (Column Phenomenex Synergi Polar-RP 100*25 mm*4 um; Condition: water(TFA)-MeCN Begin B, 22 End B 42; Gradient Time(min)7 100% B Hold Time(min)2, FlowRate (mL/min) 25), then the solvent was removed by lyophilization to afford the title compound (4.6 mg, 24% yield) as a yellow solid. ¹H NMR (CD₃OD, 400 MHz) δ [ppm]: 7.83 (d, 1H), 7.49 (d, 1H), 7.17 (d, 1H), 6.98 (d, 1H), 4.33-3.84 (m, 2H), 3.63-3.44 (m, 1H), 3.18-2.84 (m, 5H), 2.32-2.23 (m, 1H), 2.21 (s, 3H), 2.18-2.09 (m, 1H), 2.02-1.86 (m, 1H), 1.78-1.56 (m, 1H).

### Example 83

### 5-Chloro-3-methyl-2-[2-[(1-methyl-3-piperidyl)methyl]oxazolo[4,5-b]pyridin-5-yl]phenol

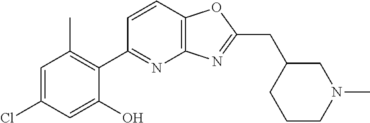 [SMILES: Cc1cc(Cl)cc(O)c1-c1ccc2oc(CC3CCCN(C)C3)nc2n1]

### Step A: 5-Bromo-2-[(1-methyl-3-piperidyl)methyl]oxazolo[4,5-b]pyridine

2-Amino-6-bromopyridin-3-ol (50.0 mg, 0.26 mmol, 1.0 eq) and 2-(1-methyl-3-piperidyl)acetic acid (46.0 mg, 0.29 mmol, 1.11 eq) were added to a solution of Eaton's Reagent (1.0 mL) at r.t. and the reaction was heated to 100° C. and left to stir for 2 hours. The reaction was left to stir overnight (16 h). The reaction mixture was poured to a stirring conical flask containing sat. NaHCO₃ (50 mL), when bubbling ceased, the solution was added to a separating funnel containing DCM (30 mL) and extracted. The separated aqueous layer was further extracted with DCM (2×30 mL). The combined organic layers were dried over Na₂SO₄, filtered, and concentrated to give the title compound (66.0 mg, 64% yield) as a dark brown solid. The compound was used without further purification in the next step. LCMS m/z: 310.0; 312.0 (Cl isotopes) [M+H]⁺, ESI pos.

### Step B: 5-Chloro-3-methyl-2-[2-[(1-methyl-3-piperidyl)methyl]oxazolo[4,5-b]pyridin-5-yl]phenol

A solution of 5-bromo-2-[(1-methyl-3-piperidyl)methyl]oxazolo[4,5-b]pyridine (66.0 mg, 0.17 mmol, 1.0 eq), (4-chloro-2-hydroxy-6-methyl-phenyl)boronic acid (CAS #1207961-50-9, 36.0 mg, 0.19 mmol, 1.13 eq) and potassium carbonate (60.0 mg, 0.43 mmol, 2.55 eq) in water (0.5 mL) and 1,4-dioxane (2.5 mL) was sparged for 5 minutes with nitrogen. [1,1′-bis(diphenylphosphino)-ferrocene]dichloropalladium(II) (7.0 mg, 0.01 mmol, 0.06 eq) was added and the reaction mixture was heated to 90° C. for 1.5 hour. The crude mixture was concentrated under reduced pressure and loaded onto silica. The crude reaction mixture was purified by column chromatography (silica gel, 24 g, 0-20% MeOH (0.7M NH₃):EtOAc) to afford the title compound (43.0 mg, 67% yield) as a light brown solid. LCMS m/z: 372.2; 374.0 (Cl isotopes) [M+H]⁺, ESI pos.

### Example 84

### Methyl 4-[(3R)-3-[[5-(4-chloro-2-hydroxy-6-methyl-phenyl)oxazolo[4,5-b]pyridin-2-yl]amino]-1-piperidyl]butanoate

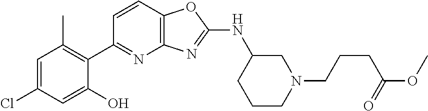 [SMILES: COC(=O)CCCN1CCCC(Nc2nc3nc(-c4c(C)cc(Cl)cc4O)ccc3o2)C1]

### Step A: tert-Butyl (3R)-3-[[5-(4-chloro-2-hydroxy-6-methyl-phenyl)oxazolo[4,5-b]pyridin-2-yl]amino]piperidine-1-carboxylate

A solution of (4-chloro-2-hydroxy-6-methyl-phenyl)boronic acid (CAS #1207961-50-9, 524 mg, 2.81 mmol, 1.12 eq), tert-Butyl (3R)-3-[(5-bromooxazolo[4,5-b]pyridin-2-yl)amino]piperidine-1-carboxylate (Example 75, Step A) (1.0 g, 2.52 mmol, 1.0 eq) and cesium carbonate (1715.0 mg, 5.26 mmol, 2.09 eq) in 1,4-Dioxane (20 mL) and Water (5 mL) was sparged (bubbling N₂ while sonicating) for 10 min. [1,1′-bis(diphenylphosphino)ferrocene]dichloropalladium(II) (95.0 mg, 0.13 mmol, 0.05 eq) was added and the reaction mixture was heated to 80° C. for 2 h. The crude mixture was filtered through Celite, rinsing with EtOAc (50 mL) and concentrated under reduced pressure. The residue was loaded onto Celite and was purified by column chromatography (C₁₈, 10-100% MeCN: 10 mM aq. NH₄HCO₃), to afford the title compound (1.16 g, 95% yield) as a light brown solid. LCMS m/z: 459.4; 461.4 (Cl isotopes) [M+H]⁺, ESI pos.

### Step B: 5-Chloro-3-methyl-2-[2-[[(3R)-3-piperidyl]amino]oxazolo[4,5-b]pyridin-5-yl]phenol;dihydrochloride

To a stirred solution of tert-butyl (3R)-3-[[5-(4-chloro-2-hydroxy-6-methyl-phenyl)oxazolo[4,5-b]pyridin-2-yl]amino]piperidine-1-carboxylate (1.16 g, 2.53 mmol, 1.0 eq) in 1,4-dioxane (22 mL) at 0° C. was added 4M HCl in dioxane (2.5 mL, 10.0 mmol, 3.96 eq) and the reaction was allowed to warm to r.t. The reaction was diluted with 1,4-dioxane (5 mL) and MeOH (5 mL) and stirred for 4 hr. The reaction mixture was concentrated under reduced pressure to afford the title compound (1.23 g, 2.85 mmol, 95% yield) as a yellow solid. LCMS m/z: 359.3; 361.3 (Cl isotopes) [M+H]⁺, ESI pos.

### Step C: Methyl 4-[(3R)-3-[[5-(4-chloro-2-hydroxy-6-methyl-phenyl)oxazolo[4,5-b]pyridin-2-yl]amino]-1-piperidyl]butanoate

5-chloro-3-methyl-2-[2-[[(3R)-3-piperidyl]amino]oxazolo[4,5-b]pyridin-5-yl]phenol;dihydro-chloride (84.0 mg, 0.19 mmol, 1.0 eq) was dissolved in triethylamine (82.0 μL, 0.59 mmol, 3.02 eq) and DCM (2.5 mL) and stirred for 5 min. Then acetic acid (56.0 μL, 0.98 mmol, 5.03 eq) was added and the mixture stirred for 1 min before the addition of methyl 4-oxobutanoate (31.0 μL, 0.3 mmol, 1.52 eq). The reaction mixture was stir for 1 h and then sodium triacetoxyborohydride (83.0 mg, 0.39 mmol, 2.01 eq) was added and the reaction mixture was stirred for a 2 h. Then methyl 4-oxobutanoate (21.0 uL, 0.2 mmol, 1.03 eq) was added, and after 0.5 h, sodium triacetoxyborohydride (42.0 mg, 0.2 mmol, 1.02 eq) was added and stirred at r.t. for 16 h. The reaction mixture was quenched with HCl (1M, 100 μL), diluted with MeOH (5 ml) and left to stir for 15 min. The reaction mixture was concentrated under reduced pressure to provide a viscous brown creamy solution. The solution was sonicated in THF (3 mL) and the resulting white solid was collected by filtration. The solid was taken up in DCM (10 mL) and K3PO4 (5 mL, 10% Aq) and stirred vigorously for 10 mins. The organic layer was separated and the aqueous was further extracted with DCM (10 mL). The combined organic layers were dried with MgSO₄, concentrated in vacuo and purified by column chromatography (silica gel, 0-10% (0.7M NH3) MeOH/DCM) to afford the title compound (30.0 mg, 33% yield) as a white solid. LCMS m/z: 459.2 [M+H]⁺, ESI pos.

### Example 85

### 3-Hydroxy-4-[2-[[(3R)-1-(2-hydroxyethyl)-3-piperidyl]amino]oxazolo[4,5-b]pyridin-5-yl]-5-methyl-benzonitrile

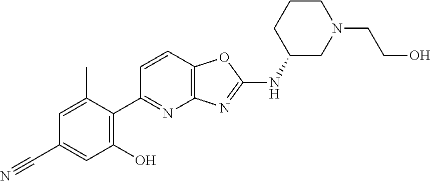 [SMILES: Cc1cc(C#N)cc(O)c1-c1ccc2oc(N[C@@H]3CCCN(CCO)C3)nc2n1]

The title compound was obtained as a dark brown foam, LCMS: m/z 394.3 [M+H]⁺, ESI pos, using chemistry similar to that described in Example 14, step 2 starting from 2-[(3R)-3-[(5-chlorooxazolo[4,5-b]pyridin-2-yl)amino]-1-piperidyl]ethanol (Example 52, step 2) and (4-cyano-2-hydroxy-6-methyl-phenyl)boronic acid (Example 25, step D).

### Example 86

### Methyl 3-[(3R)-3-[[5-(4-chloro-2-hydroxy-6-methyl-phenyl)oxazolo[4,5-b]pyridin-2-yl]amino]-1-piperidyl]propanoate

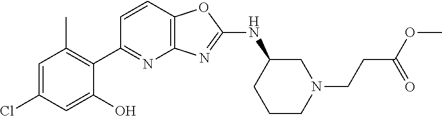 [SMILES: COC(=O)CCN1CCC[C@@H](Nc2nc3nc(-c4c(C)cc(Cl)cc4O)ccc3o2)C1]

### Step A: Methyl 3-[(3R)-3-[(5-bromooxazolo[4,5-b]pyridin-2-yl)amino]-1-piperidyl]propanoate

To a stirred solution of 5-bromo-N-[(3R)-3-piperidyl]oxazolo[4,5-b]pyridin-2-amine;dihydrochloride (50.0 mg, 0.12 mmol, 1.0 eq) (Example 75, step B) in MeCN (2.4 mL) was added cesium carbonate (310.0 mg, 0.95 mmol, 4.0 eq) and the reaction mixture was sonicated (2 min) and left to stir for 0.5 h. Then, methyl 3-bromopropionate (29.0 μL, 0.27 mmol, 1.12 eq) was added and the reaction was left to stir for 1 h. The reaction was then heated at 60° C. for 2 h. Afterwards, the reaction was filtered through Celite, concentrated under reduced pressure and loaded onto Celite. The crude reaction mixture was purified by reverse phase column chromatography (dry load, C₁₈, 15-60% MeCN:H₂O (0.1% NH4HCO₃)) to yield the title compound (60.0 mg, 53% yield) as an off-white glass. LCMS m/z: 383.2 (⁷⁹Br); 385.2 (⁸¹Br) [M+H]⁺, ES pos.

### Step B: Methyl 3-[(3R)-3-[[5-(4-chloro-2-hydroxy-6-methyl-phenyl)oxazolo[4,5-b]pyridin-2-yl]amino]-1-piperidyl]propanoate

Following the procedures outlined in example 95, aforementioned methyl 3-[(3R)-3-[(5-bromooxazolo[4,5-b]pyridin-2-yl)amino]-1-piperidyl]propanoate and (4-chloro-2-hydroxy-6-methyl-phenyl)boronic acid (CAS #1207961-50-9) were reacted to afford the title compound. LCMS m/z: 445.3; 447.3 (Cl isotopes) [M+H]⁺, ESI pos.

### Example 87

### 5-Chloro-2-[2-[[6-(hydroxymethyl)-1-methyl-3-piperidyl]amino]oxazolo[4,5-b]pyridin-5-yl]-3-methyl-phenol;2,2,2-trifluoroacetic acid

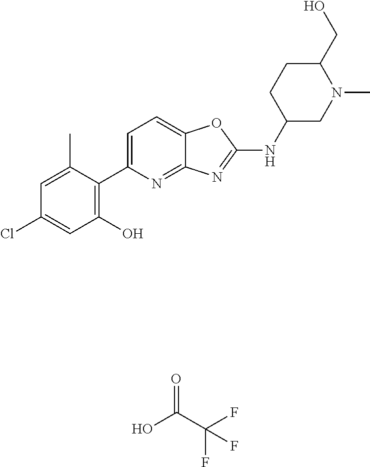 [SMILES: Cc1cc(Cl)cc(O)c1-c1ccc2oc(NC3CCC(CO)N(C)C3)nc2n1 ; O=C(O)C(F)(F)F]

### Step A: 5-((tert-Butoxycarbonyl)amino)-2-(hydroxymethyl)-1-methylpyridin-1-ium (HI salt)

To a solution of tert-butyl (6-(hydroxymethyl)pyridin-3-yl) carbamate (100 mg, 0.45 mmol, 1.0 eq) (CAS: 323578-38-7) in MeCN (2 mL) was added iodomethane (189.9 mg, 1.34 mmol, 3.0 eq). The mixture was stirred at 70° C. for 5 hours. The reaction mixture was cooled to 20° C. and concentrated in vacuum. The residue was triturated in ethyl acetate (3 ml) and stirred for 30 minutes. The suspension was filtered and the filter cake was washed with ethyl acetate (2 ml×2), dried in vacuum to afford the title compound (100.0 mg, 0.42 mmol, 94% yield, HI salt) as yellow solid. LCMS: 239.0 [M+H]⁺, ESI pos.

### Step B: tert-Butyl (6-(hydroxymethyl)-1-methylpiperidin-3-yl)carbamate

To a solution of 5-((tert-butoxycarbonyl)amino)-2-(hydroxymethyl)-1-methylpyridin-1-ium (100.0 mg, 0.42 mmol, 1.0 eq) in methanol (5 mL) was added PtO₂ (10 mg, 0.04 mmol, 0.1 eq) under N₂. The suspension was degassed under vacuum and purged with H₂ several times. Then the mixture was stirred under 2280 mmHg of H₂ pressure for 48 hours at 25° C. The suspension was filtered and the filter cake was washed with MeOH (20 mL×3). The combined filtrate was concentrated in vacuum to afford the title compound (100 mg, 98% yield) as yellow oil. ¹H NMR (400 MHz, DMSO-d₆) δ 7.98 (s, 1H), 3.95-3.85 (m, 1H), 3.84-3.77 (m, 1H), 3.64 (dd, 1H), 3.00 (s, 3H), 2.84-2.80 (m, 1H), 2.75-2.70 (m, 2H), 2.05-1.84 (m, 2H), 1.83-1.60 (m, 2H), 1.44 (s, 9H).

### Step C: (5-Amino-1-methylpiperidin-2-yl)methanol (HCl salt)

A mixture of tert-butyl (6-(hydroxymethyl)-1-methylpiperidin-3-yl)carbamate (100.0 mg, 0.41 mmol, 1.0 eq) in 1,4-dioxane (2 mL) was added HCl/MeOH (2.0 mL). The mixture was stirred at 20° C. for 12 hours. Afterwards, the reaction mixture was concentrated in vacuum to afford the title compound (70.0 mg, 0.39 mmol, 95% yield, HCl salt) as yellow oil. ¹H NMR (400 MHz, CD₃OD) δ 4.10-3.95 (m, 1H), 3.92-3.80 (m, 2H), 3.75-3.50 (m, 1H), 3.43-3.37 (m, 1H), 3.25-3.15 (m, 1H), 3.05 (s, 3H), 2.12-2.08 (m, 2H), 2.05-1.98 (m, 2H) 1.85-1.75 (m, 1H).

### Step D: (5-((5-Bromooxazolo[4,5-b]pyridin-2-yl) amino)-1-methylpiperidin-2-yl) methanol (TFA salt)

To a solution of (5-amino-1-methylpiperidin-2-yl)methanol (55.72 mg, 0.31 mmol, 1.2 eq) in NMP (1 mL) was added DIPEA (99.46 mg, 0.77 mmol, 3.0 eq) and 5-bromo-2-chlorooxazolo[4,5-b]pyridine (60.0 mg, 0.26 mmol, 1.0 eq) in portions. The mixture was stirred at 20° C. for 1 hour. The residue was purified by column chromatography (C₁₈, 0.1% TFA in water/MeCN) to afford the title compound (90.0 mg, 77% yield, TFA salt) as yellow oil. ¹H NMR (400 MHz, CD₃OD) δ 7.59 (d, 1H), 7.25 (d, 1H), 4.25-4.15 (m, 1H), 4.10-4.00 (m, 2H), 3.67-3.61 (m, 1H), 3.42-3.35 (m, 1H), 2.94 (s, 3H), 2.40-2.25 (m, 2H), 2.15-2.07 (m, 2H) 1.95-1.85 (m, 1H).

### Step E: 5-Chloro-2-[2-[[6-(hydroxymethyl)-1-methyl-3-piperidyl]amino]oxazolo[4,5-b]pyridin-5-yl]-3-methyl-phenol;2,2,2-trifluoroacetic acid

To a solution of (5-((5-bromooxazolo[4,5-b]pyridin-2-yl)amino)-1-methylpiperidin-2-yl) methanol (80.0 mg, 0.18 mmol, 1.0 eq) and (4-chloro-2-hydroxy-6-methylphenyl)boronic acid (49.1 mg, 0.26 mmol, 1.5 eq) in 1,4-dioxane (3 mL) and water (0.6 mL) was added K₂CO₃ (72.86 mg, 0.53 mmol, 3.0 eq) and XPhos Pd G3 (14.9 mg, 0.02 mmol, 0.1 eq) under N₂. The mixture was stirred at 80° C. for 2 hours under N₂. Then, the mixture was cooled to 20° C. and concentrated in vacuum. The residue was dissolved with MeOH (2 ml) and purified by prep-HPLC (Column: Phenomenex Synergi Polar-RP 100*25 mm*4 um, Condition: water(TFA)-MeCN, Begin B: 22; End B: 42; Gradient Time(min): 7; 100% B Hold Time(min): 2; FlowRate (ml/min): 25) to afford the title compound (58.3 mg, 62% yield, TFA salt) as yellow solid. LCMS: m/z 403.2 [M+H]⁺, ESI pos.

### Example 88

### 5-Chloro-2-[2-[(4-isopropylmorpholin-2-yl)methyl]oxazolo[4,5-b]pyridin-5-yl]-3-methyl-phenol;formic acid

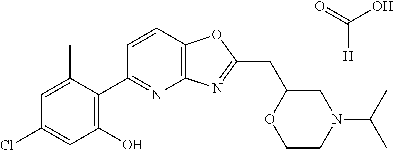

### Step A: 5-Bromo-2-[(4-isopropylmorpholin-2-yl)methyl]oxazolo[4,5-b]pyridine

2-amino-6-bromopyridin-3-ol (50.0 mg, 0.26 mmol, 1.0 eq) and 2-(4-isopropylmorpholin-2-yl)acetic acid (54.0 mg, 0.29 mmol, 1.09 eq) were added to a solution of Eaton's Reagent (1.0 mL) at r.t. and the reaction was heated to 100° C. and left to stir overnight. The reaction mixture was poured to a stirring conical flask containing sat. NaHCO₃ (50 mL), when bubbling ceased, the solution was added to a separating funnel containing DCM (30 mL) and extracted. The separated aqueous layer was further extracted with DCM (2×30 mL). The combined organic layers were dried over Na₂SO₄, filtered, and concentrated to give the title compound (84.0 mg, 71% yield) as a dark brown solid. LCMS m/z: 340.0; 342.0 (Cl isotopes) [M+H]⁺, ESI pos.

### Step B: 5-Chloro-2-[2-[(4-isopropylmorpholin-2-yl)methyl]oxazolo[4,5-b]pyridin-5-yl]-3-methyl-phenol;formic acid

A solution of 5-bromo-2-[(4-isopropylmorpholin-2-yl)methyl]oxazolo[4,5-b]pyridine (84.0 mg, 0.19 mmol, 1.0 eq), (4-chloro-2-hydroxy-6-methyl-phenyl)boronic acid (39.0 mg, 0.21 mmol, 1.12 eq) and potassium carbonate (65.0 mg, 0.47 mmol, 2.51 eq) in water (0.5 mL) and 1,4-dioxane (2 mL) was sparged with N2 for 10 min. [1,1′-bis(diphenylphosphino)ferrocene]-dichloropalladium(II) (7.0 mg, 0.01 mmol, 0.05 eq) was added and the reaction mixture was heated to 90° C. for 70 min. The reaction was left to stir overnight. The crude mixture concentrated under reduced pressure and purified by column chromatography (silica gel, 0-12% MeOH (0.7M NH₃):EtOAc) to provide product with co-eluted starting material. A second reverse-phase column was carried out (dry load celite; C₁₈, 10-60% MeCN (0.1% formic acid): 0.1% aq. formic acid to afford the title compound (25.0 mg, 28% yield) as a light brown solid. LCMS m/z: 402.2; 404.2 (Cl isotopes) [M+H]⁺, ESI pos.

### Example 89

### 3-Methyl-2-[2-[[(3R)-1-methyl-3-piperidyl]amino]oxazolo[4,5-b]pyridin-5-yl]-5-methylsulfonyl-phenol;2,2,2-trifluoroacetic acid

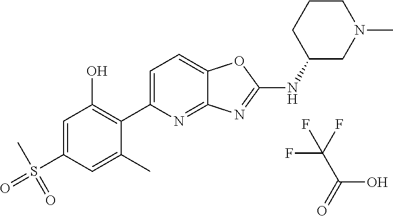

### Step A: 3-Methyl-5-(methylsulfonyl)phenol

The apparatus was dried by heating with a heat gun under vacuum. To a mixture of 3-bromo-5-methylphenol (4500.0 mg, 24.06 mmol, 1.0 eq CAS: 74204-00-5) in DMSO (45 mL) was added CuI (1142.86 mg, 6.02 mmol, 0.25 eq), L-Proline (1385.0 mg, 12.03 mmol, 0.5 eq) and sodium hydroxide (481.17 mg, 12.03 mmol, 0.5 eq) and sodium methanesulfinate (7362.5 mg, 72.2 mmol, 3.0 eq), and the mixture was stirred at 120° C. for 3 h under N₂. The reaction mixture was quenched with water (20 mL), then adjust pH=8 by 1N HCl solution, then extracted by EtOAc (30 mL×3), dry over anhydrous Na₂SO₄, filtrated and the filtrate was concentrated under reduce pressure. The crude was purified by column chromatography (silica gel, petroleum ether/ethyl acetate=10/1 to 1/1) to give the title compound (3000 mg, 67% yield) was as yellow oil. ¹H NMR (CD₃OD, 400 MHz) δ 7.21 (s, 1H), 7.11 (s, 1H), 6.92 (s, 1H), 3.06 (s, 3H), 2.36 (s, 3H).

### Step B: 2-Iodo-3-methyl-5-methylsulfonyl-phenol

To a mixture of 3-methyl-5-methylsulfonyl-phenol (3.0 g, 16.11 mmol, 1.0 eq) in toluene (25 mL) was added NaH (1.29 g, 32.22 mmol, 2.0 eq 60%) at 0° C., and the mixture was stirred at 0° C. for 10 mins, then I₂ (3.26 g, 12.89 mmol, 0.8 eq) was added to the mixture and the mixture was stirred at 25° C. for 12 h. The reaction mixture was quenched with water, then adjust pH=6 by 1N HCl solution, then extracted by EtOAc (20 mL×3), washed with brine (20 mL×2), dry over anhydrous Na₂SO₄, filtrated and the filtrate was concentrated to get a yellow solid. The residue was purified by column chromatography (silica gel, petroleum ether/ethyl acetate=10:0 to 1:1) to afford the title compound (1.3 g, 26% yield) as a yellow solid. LCMS: m/z 312.9 [M+H]⁺, ESI pos.

### Step C: 3-Methyl-5-methylsulfonyl-2-(4,4,5,5-tetramethyl-1,3,2-dioxaborolan-2-yl)phenol and (2-hydroxy-6-methyl-4-methylsulfonyl-phenyl)boronic acid

To a mixture of 2-iodo-3-methyl-5-methylsulfonyl-phenol (400.0 mg, 1.28 mmol, 1.0 eq), 4,4,4′,4′,5,5,5′,5′-octamethyl-2,2′-bi(1,3,2-dioxaborolane) (1.62 g, 6.41 mmol, 5.0 eq) in 1,4-dioxane (8 mL) was added KOAc (377.3 mg, 3.84 mmol, 3.0 eq) and Pd(dppf)Cl₂ (93.7 mg, 0.13 mmol, 0.1 eq), then the mixture was stirred at 95° C. for 4 hours. The mixture was cooled to 25° C. The reaction mixture was filtered, and the filtrate was concentrated under vacuum. The crude was purified by column chromatography (C₁₈, 0.1% TFA in water/MeCN) to afford the title compound (100 mg, 25% yield) as a yellow solid. LCMS: m/z 313.3 [M+H]⁺, ESI pos.

### Step D: 3-Methyl-2-[2-[[(3R)-1-methyl-3-piperidyl]amino]oxazolo[4,5-b]pyridin-5-yl]-5-methylsulfonyl-phenol;2,2,2-trifluoroacetic acid

To a solution of 3-methyl-5-methylsulfonyl-2-(4,4,5,5-tetramethyl-1,3,2-dioxaborolan-2-yl)phenol (50.0 mg, 0.16 mmol, 1.0 eq), 5-bromo-N-[(3R)-1-methyl-3-piperidyl]oxazolo[4,5-b]pyridin-2-amine;2,2,2-trifluoroacetic acid (Example 30, step H) (68.1 mg, 0.16 mmol, 1.0 eq), K₂CO₃ (66.31 mg, 0.48 mmol, 3.0 eq), in 1,4-dioxane (1 mL)/water (0.2 mL) was added Pd(dppf)Cl₂ (11.71 mg, 0.02 mmol, 0.1 eq) under N2 at 25° C., then stirred for 4 hours at 95° C. The mixture was then cooled to 25° C., filtered, and the filtrate was concentrated under vacuum. The crude was purified by C18 column chromatography (20 g, 0.1% TFA in water/MeCN), to afford the title compound (35.9 mg, 42% yield) as a white solid. LCMS: m/z 417.1 [M+H]⁺, ESI pos.

### Example 90

### 5-Chloro-2-[2-[2-methoxyethyl-[(3R)-1-methyl-3-piperidyl]amino]oxazolo[4,5-b]pyridin-5-yl]-3-methyl-phenol;formic acid

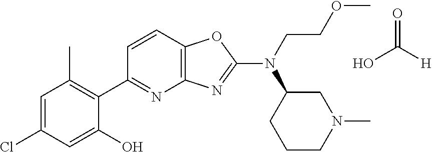

### Step A: 5-Chloro-N-(2-methoxyethyl)-N-[(3R)-1-methyl-3-piperidyl]oxazolo[4,5-b]pyridin-2-amine

A solution of 5-chloro-N-[(3R)-1-methyl-3-piperidyl]oxazolo[4,5-b]pyridin-2-amine (Example 15, step 1) (200 mg, 0.75 mmol, 1.0 eq) in DMF (2 mL) was cooled to 0° C. and sodium hydride (60% in mineral oil, 45 mg, 1.12 mmol, 1.5 eq) added. The mixture was allowed to warm to r.t. and stirred for 5 mins. Then 2-bromoethyl methyl ether (CAS #6482-24-2, 0.08 mL, 0.9 mmol, 1.2 eq) was added to the reaction mixture which was stirred at rt for 16 hrs. The reaction mixture was diluted with water (10 mL) and EtOAc (10 mL). The phases were separated and the aqueous was extracted using further EtOAc (2×10 mL). The combined organic extracts were washed with brine (2×10 mL), dried (MgSO4), filtered and concentrated in vacuo. The crude product was purified by chromatography on silica gel (12 g, 0-10% (0.7 M Ammonia/MeOH)/DCM) to afford the title compound (65.0 mg, 25% yield) as a colorless gum. LCMS m/z 325.3 [M+H]⁺, ESI pos.

### Step B: 5-Chloro-2-[2-[2-methoxyethyl-[(3R)-1-methyl-3-piperidyl]amino]oxazolo[4,5-b]pyridin-5-yl]-3-methyl-phenol;formic acid

A mixture of 5-chloro-N-(2-methoxyethyl)-N-[(3R)-1-methyl-3-piperidyl]oxazolo[4,5-b]pyridin-2-amine (65.0 mg, 0.2 mmol, 1.0 eq), (4-chloro-2-hydroxy-6-methyl-phenyl)boronic acid (41.0 mg, 0.22 mmol, 1.1 eq), 1,1′-bis(diphenylphosphino)ferrocenepalladium(II) dichloride toluene adduct (8.2 mg, 0.01 mmol, 0.05 eq) and potassium carbonate (55.3 mg, 0.4 mmol, 2.0 eq) in 1,4-Dioxane (1.6 mL) and water (0.4 mL) was degassed with N₂ for ˜5 min, then was heated to 80° C. for 4 h. The mixture was cooled to rt, after which XPhos Pd G3 (8.5 mg, 0.01 mmol, 0.05 eq) was added and the reaction mixture degassed with N₂ for ˜5 mins and then heated to 80° C. for 3 h. The reaction mixture was cooled to rt and stirred at this temperature for 2 days. The reaction mixture was diluted with brine (10 mL) and EtOAc (10 mL), after which the layers were separated, and the aqueous layer was further extracted with EtOAc (2×10 mL). The combined organics were concentrated in vacuo and the crude product was purified by silica gel chromatography (4 g, 0-10% ((0.7M NH₃ in MeOH)/DCM)) and then by RP chromatography (12 g, 5-55% (0.1% formic acid in MeCN)/(0.1% formic acid in water)) to afford the title compound (26.0 mg, 26% yield) as a pale yellow solid. LCMS m/z 431.4 [M+H]⁺, ESI pos.

### Example 91

### 2-[2-[[(3R)-1-Ethylazepan-3-yl]amino]oxazolo[4,5-b]pyridin-5-yl]-3-methyl-5-(trifluoromethyl)phenol

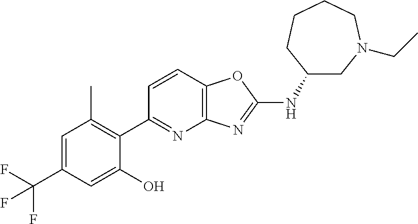 [SMILES: CCN1CCCC[C@@H](Nc2nc3nc(-c4c(C)cc(C(F)(F)F)cc4O)ccc3o2)C1]

### Step 1: tert-Butyl N-[(3R)-1-ethylazepan-3-yl]carbamate

The title compound was obtained as a light yellow oil, LCMS: m/z 243.4 [M+H]⁺, ESI pos, using chemistry similar to that described in Example 46, step 1 starting from tert-butyl N-[(3R)-azepan-3-yl]carbamate (CAS #1354351-56-6) and iodoethane (CAS #75-03-6).

### Step 2: 5-Chloro-N-[(3R)-1-ethylazepan-3-yl]oxazolo[4,5-b]pyridin-2-amine

The title compound was obtained as a light yellow solid, LCMS: m/z 295.3 [M+H]⁺, ESI pos, using chemistry similar to that described in Example 52, step 2 starting from tert-butyl N-[(3R)-1-ethylazepan-3-yl]carbamate (Example 91, step 1) and 5-chloro-2-(methylthio)oxazolo[4,5-b]pyridine (CAS #1783370-92-2).

### Step 3: 2-[2-[[(3R)-1-Ethylazepan-3-yl]amino]oxazolo[4,5-b]pyridin-5-yl]-3-methyl-5-(trifluoromethyl)phenol

The title compound was obtained as a yellow foam, LCMS: m/z 435.3 [M+H]⁺, ESI pos, using chemistry similar to that described in Example 14, step 2 starting from 5-chloro-N-[(3R)-1-ethylazepan-3-yl]oxazolo[4,5-b]pyridin-2-amine (Example 91, step 1) and 3-methyl-2-(4,4,5,5-tetramethyl-1,3,2-dioxaborolan-2-yl)-5-(trifluoromethyl)phenol (CAS #2557358-38-8).

### Example 92

### 5-Chloro-2-[2-[(3-hydroxycyclobutyl)methylamino]oxazolo[4,5-b]pyridin-5-yl]-3-methyl-phenol

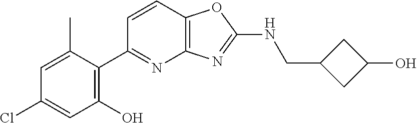 [SMILES: Cc1cc(Cl)cc(O)c1-c1ccc2oc(NCC3CC(O)C3)nc2n1]

### Step A: 3-(Aminomethyl)cyclobutanol;hydrochloride

To a solution of trans-3-(Boc-aminomethyl)cyclobutanol (100 mg, 0.5 mmol, 1.0 eq) in 1,4-dioxane (0.5 mL) was added HCl/dioxane (0.5 mL, 2.0 mmol, 4.03 eq). The mixture was stirred at 20° C. for 2 hrs. Afterwards, the mixture was concentrated under reduced pressure to yield the title compound (68.0 mg, 99% yield) as a white solid.

### Step B: 3-[[(5-Bromooxazolo[4,5-b]pyridin-2-yl)amino]methyl]cyclobutanol;2,2,2-trifluoro-acetic acid

To a solution of 3-(aminomethyl)cyclobutanol;hydrochloride (68.0 mg, 0.49 mmol, 1.0 eq) and DIPEA (0.26 mL, 1.48 mmol, 3.0 eq) in MeCN (7 mL) was added 5-bromo-2-chloro-oxazolo[4,5-b]pyridine (115.4 mg, 0.49 mmol, 1.0 eq) in five portions and the mixture was stirred at 20° C. for 1 h. The reaction mixture was filtered and the filtrate was concentrated under vacuum to give the residue which was purified by column chromatography (Cis, 0.1% TFA in water/MeCN) to yield the title compound (45.0 mg, 31% yield) as white foam. LCMS: m/z 298.1 [M+H]⁺, ESI pos.

### Step C: 5-Chloro-2-[2-[(3-hydroxycyclobutyl)methylamino]oxazolo[4,5-b]pyridin-5-yl]-3-methyl-phenol

To a solution of 3-[[(5-bromooxazolo[4,5-b]pyridin-2-yl)amino]methyl]cyclobutanol;2,2,2-trifluoroacetic acid (20.0 mg, 0.07 mmol, 1.0 eq) and (4-chloro-2-hydroxy-6-methyl-phenyl)boronic acid (18.76 mg, 0.1 mmol, 1.5 eq) in 1,4-dioxane (2 mL) and water (0.4 mL) was added CsF (40.8 mg, 0.27 mmol, 4.0 eq) and XPhosPdG3 (5.68 mg, 0.01 mmol, 0.1 eq) under nitrogen atmosphere. The mixture was stirred at 90° C. for 2 hours. Afterwards, the reaction mixture was cooled to 25° C., filtered and the filtrate was concentrated in vacuum. The crude product was purified by column chromatography (Cis, 0.1% TFA in water/MeCN) to give the title compound (14.5 mg, 58% yield) as a white solid. LCMS: m/z 360.0 [M+H]⁺, ESI pos.

### Example 93

### 5-Chloro-2-[2-[[(3R)-1-(2-hydroxyethyl)-3-piperidyl]amino]oxazolo[4,5-b]pyridin-5-yl]-3-methyl-phenol

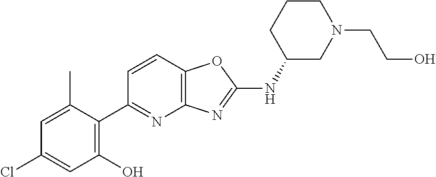 [SMILES: Cc1cc(Cl)cc(O)c1-c1ccc2oc(N[C@@H]3CCCN(CCO)C3)nc2n1]

The title compound was obtained as an off-white foam, LCMS: m/z 403.2 [M+H]⁺, ESI pos, using chemistry similar to that described in Example 19, starting from 2-[(3R)-3-[(5-chlorooxazolo[4,5-b]pyridin-2-yl)amino]-1-piperidyl]ethanol (Example 52, step 2) and (4-chloro-2-hydroxy-6-methyl-phenyl)boronic acid (CAS #1207961-50-9).

### Example 94

### 5-Chloro-3-(hydroxymethyl)-2-[2-[[(3R)-1-methyl-3-piperidyl]amino]oxazolo[4,5-b]pyridin-5-yl]phenol

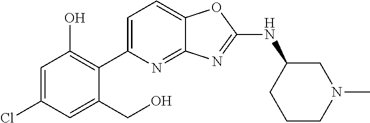 [SMILES: CN1CCC[C@@H](Nc2nc3nc(-c4c(O)cc(Cl)cc4CO)ccc3o2)C1]

### Step A: 5-Chloro-3-hydroxy-2-iodo-benzaldehyde

To a mixture of 3-chloro-5-hydroxybenzaldehyde (CAS #1829-33-0, 8.1 g, 51.7 mmol, 1.0 eq,) in DMF (81 mL) was added NaH (2.48 g, 103.47 mmol, 2.0 eq 60%) portion wise at 0° C. under N2, stirred for 30 mins, then I₂ (13.1 g, 51.7 mmol, 1.0 eq) was added portion wise and stirred for 15.5 hours at 25° C. The mixture was poured into one mole of hydrochloric acid in aqueous solution (100 ml) and extracted with ethyl acetate (100 mL×3). The combined organic layers were washed with brine (150 mL), dried over anhydrous sodium sulfate, filtered, and the filtrate was concentrated under reduced pressure. The crude was purified by silica gel column chromatography (petroleum ether:ethyl acetate=1:0 to 5:1) and re-purified by column chromatography (Cis, 20 g, 0.1% NH₃H₂O in water/MeCN) to yield the title compound (730 mg, 4% yield) as a light green solid. LCMS: m/z 280.9 [M−H]⁻, ESI neg.

### Step B: 5-Chloro-2-iodo-3-(2-trimethylsilylethoxymethoxy)benzaldehyde

A mixture of Cs₂CO₃ (865.13 mg, 2.66 mmol, 1.5 eq) and 5-chloro-3-hydroxy-2-iodo-benzaldehyde (500.0 mg, 1.77 mmol, 1.0 eq) in DMF (10 mL) was degassed and purged with N₂ three times and stirred for 30 mins. Then, SEM-Cl (442.7 mg, 2.66 mmol, 1.5 eq) was added to the mixture in one portion. The mixture was stirred at 25° C. for 2 hrs. Afterwards, the mixture was poured into water (50 mL) and extracted with ethyl acetate (40 mL×3). The combined organic layers were washed with brine (20 mL), dried over anhydrous sodium sulfate, filtered, and the filtrate was concentrated under reduced pressure. The crude was purified by column chromatography (silica gel, petroleum ether:ethyl acetate=1:0 to 10:1) to give the title compound (500 mg, 68% yield) as yellow oil. ¹H NMR (400 MHz, DMSO-d₆) δ [ppm]: 10.02 (s, 1H), 7.48 (d, 1H), 7.38 (d, 1H), 5.46 (s, 2H), 3.76 (t, 2H), 0.89 (t, 2H), −0.04 (s, 9H).

### Step C: [5-Chloro-2-iodo-3-(2-trimethylsilylethoxymethoxy)phenyl]methanol

Sodium borohydride (55.0 mg, 1.45 mmol, 1.2 eq) was added in small portions to a solution of 5-chloro-2-iodo-3-(2-trimethylsilylethoxymethoxy)benzaldehyde (500.0 mg, 1.21 mmol, 1.0 eq) in methanol (5 mL) and water (0.5 mL). The mixture was stirred for 3 hours at 25° C. The mixture was poured water (15 ml) and extracted with ethyl acetate (30 mL×3). The combined organic layers were washed with brine (20 mL), dried over anhydrous sodium sulfate, filtered, and the filtrate was concentrated under reduced pressure to give the title compound (470 mg, 94% yield) as yellow oil. ¹H NMR (400 MHz, DMSO-d₆) δ [ppm]: 7.13 (s, 1H), 7.09 (d, 1H), 5.60 (t, 1H), 5.36 (s, 2H), 4.42-4.37 (m, 2H), 3.73 (t, 2H), 0.92-0.85 (m, 2H), −0.02-0.05 (m, 9H).

### Step D: [4-Chloro-2-(hydroxymethyl)-6-(2-trimethylsilylethoxymethoxy)phenyl]boronic acid

A mixture of [5-chloro-2-iodo-3-(2-trimethylsilylethoxymethoxy)phenyl]methanol (60.0 mg, 0.14 mmol, 1.0 eq) and bis(pinacolato)diboron (183.69 mg, 0.72 mmol, 5.0 eq) in 1,4-dioxane (150 mL) was degassed and purged with nitrogen three times and KOAc (42.6 mg, 0.43 mmol, 3.0 eq), Pd(dppf)Cl₂ (10.6 mg, 0.01 mmol, 0.1 eq) was added to the mixture in one portion. The mixture was heated under microwave irradiation at 90° C. for 2 h. The mixture was purified by column chromatography (Cis, 0.1% TFA in water/MeCN) to yield the title compound (40.0 mg, 0.1 mmol, 67% yield) as dark brown oil. ¹H NMR (400 MHz, CD₃OD) δ [ppm]: 6.99 (s, 1H), 6.94 (s, 1H), 5.29 (s, 1H), 5.22 (s, 1H), 4.55 (s, 2H), 3.81-3.71 (m, 2H), 0.93 (t, 2H), 0.01 (s, 9H).

### Step E: [5-Chloro-2-[2-[[(3R)-1-methyl-3-piperidyl]amino]oxazolo[4,5-b]pyridin-5-yl]-3-(2-trimethylsilylethoxymethoxy)phenyl]methanol

A mixture of K₂CO₃ (41.5 mg, 0.3 mmol, 2.5 eq), 5-bromo-N-[(3R)-1-methyl-3-piperidyl]oxazolo[4,5-b]pyridin-2-amine;2,2,2-trifluoroacetic acid (76.7 mg, 0.18 mmol, 1.5 eq) and [4-chloro-2-(hydroxymethyl)-6-(2-trimethylsilylethoxymethoxy)phenyl]boronic acid (40.0 mg, 0.12 mmol, 1.0 eq) in 1,4-dioxane (1 mL) and water (0.2 mL) was degassed and purged with nitrogen three times and XPhos Pd G3 (10.11 mg, 0.01 mmol, 0.1 eq) was added to the mixture. The mixture was heated at 95° C. for 2 h. The mixture was concentrated under reduced pressure and purified by column chromatography (Cis, 0.1% NH₃H₂O in water/MeCN) to yield the title compound (27.0 mg, 43% yield) as a red oil. ¹H NMR (400 MHz, CD₃OD) δ [ppm]: 7.80 (d, 1H), 7.28 (s, 1H), 7.23 (s, 1H), 7.13 (d, 1H), 5.13 (s, 2H), 4.34 (s, 1H), 4.15-4.09 (m, 1H), 3.88 (d, 1H), 3.57 (t, 2H), 3.54-3.47 (m, 1H), 2.94 (s, 3H), 2.32-2.19 (m, 1H), 2.18 (br s, 1H), 2.03 (s, 1H), 1.93-1.84 (m, 1H), 1.72-1.64 (m, 1H), 1.42-1.27 (m, 1H), 0.85 (t, 2H), −0.05 (s, 9H).

### Step F: 5-Chloro-3-(hydroxymethyl)-2-[2-[[(3R)-1-methyl-3-piperidyl]amino]oxazolo[4,5-b]pyridin-5-yl]phenol

A solution of [5-chloro-2-[2-[[(3R)-1-methyl-3-piperidyl] amino]oxazolo[4,5-b]pyridin-5-yl]-3-(2-trimethylsilylethoxymethoxy)phenyl]methanol (14.0 mg, 0.03 mmol, 1.0 eq) and TFA (0.26 mL, 3.49 mmol, 129.4 eq) in DCM (1 mL) was stirred at 25° C. for 2 h. After removal of the solvent, the residue was purified by column chromatography (C₁₈, 0.1% TFA in water/MeCN) to yield the title compound (7.25 mg, 0.01 mmol, 69% yield) as a yellow solid. ¹H NMR (400 MHz, CD₃OD) δ [ppm]: 7.87 (d, 1H), 7.27 (d, 1H), 7.10 (s, 1H), 6.90 (s, 1H), 4.50-4.32 (m, 2H), 4.29-4.09 (m, 1H), 3.98-3.79 (m, 1H), 3.64-3.38 (m, 1H), 3.15-3.05 (m, 1H), 2.94 (s, 5H), 2.29-2.08 (m, 1H), 2.03-1.82 (m, 2H), 1.79-1.59 (m, 1H).

### Example 95

### 5-Chloro-2-[2-[[(3R)-1-(3-hydroxypropyl)-3-piperidyl]amino]oxazolo[4,5-b]pyridin-5-yl]-3-methyl-phenol

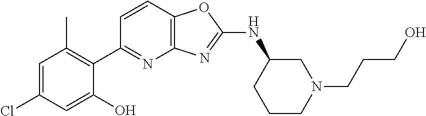 [SMILES: Cc1cc(Cl)cc(O)c1-c1ccc2oc(N[C@@H]3CCCN(CCCO)C3)nc2n1]

### Step A: 3-[(3R)-3-[(5-bromooxazolo[4,5-b]pyridin-2-yl)amino]-1-piperidyl]propan-1-ol

To a stirred solution of 5-bromo-N-[(3R)-3-piperidyl]oxazolo[4,5-b]pyridin-2-amine;dihydrochloride (Example 75, step B) (50.0 mg, 0.12 mmol, 1.0 eq) in 1,4-dioxane (1.2 mL) was added N,N-diisopropylethylamine (200 μL, 1.15 mmol, 9.66 eq) and 3-bromo-1-propanol (12.0 μL, 0.13 mmol, 1.12 eq) and the reaction was left to stir at 100° C. for 18 hr. The reaction was cooled to r.t. and diluted with DCM (5 mL) and MeOH (1 mL) and then concentrated under reduced pressure onto silica. The crude reaction mixture was purified by column chromatography (silica gel, 0-10% MeOH (0.7 M NH₃):EtOAc, to afford the title compound (22 mg, 60% yield) as a light yellow oil. LCMS m/z 355.2.1; 357.2 (Br isotopes) [M+H]⁺, ESI pos.

### Step B: 5-Chloro-2-[2-[[(3R)-1-(3-hydroxypropyl)-3-piperidyl]amino]oxazolo[4,5-b]pyridin-5-yl]-3-methyl-phenol

A solution of 3-[(3R)-3-[(5-bromooxazolo[4,5-b]pyridin-2-yl)amino]-1-piperidyl]propan-1-ol (55.0 mg, 0.15 mmol, 1.0 eq), (4-chloro-2-hydroxy-6-methyl-phenyl)boronic acid (33.0 mg, 0.18 mmol, 1.14 eq) and potassium carbonate (43.0 mg, 0.31 mmol, 2.01 eq) in 1,4-dioxane (3 mL) and water (1 mL) was sparged (bubbling N₂ while sonicating) for 10 min. [1,1′-bis(diphenylphosphino)ferrocene]dichloropalladium(II) (7.0 mg, 0.01 mmol, 0.06 eq) was added and the reaction mixture was heated to 90° C. for 16 h. The crude mixture was concentrated under reduced pressure and purified by column chromatography (silica gel, 0-50% MeOH:EtOAc ) to afford the title compound (39.0 mg, 59% yield) as a dark brown solid. LCMS m/z m/z 417.4 [M+H]⁺, ESI pos.

### Example 96

### 5-Chloro-2-[2-[[(3R)-1-(3-methoxypropyl)-3-piperidyl]amino]oxazolo[4,5-b]pyridin-5-yl]-3-methyl-phenol

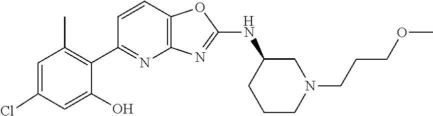

### Step A: 5-Bromo-N-[(3R)-1-(3-methoxypropyl)-3-piperidyl]oxazolo[4,5-b]pyridin-2-amine

To a stirred solution of 5-bromo-N-[(3R)-3-piperidyl]oxazolo[4,5-b]pyridin-2-amine;dihydrochloride (50.0 mg, 0.12 mmol, 1.0 eq) (Example 73, step 3) in MeCN (2.4 mL) was added cesium carbonate (315.0 mg, 0.97 mmol, 4.07 eq) and the reaction mixture was sonicated (2 min) and left to stir for 0.5 h. Then, 1-bromo-3-methoxypropane (29.0 μL, 0.26 mmol, 1.1 eq) was added and the reaction was left to stir for 1 h. Afterwards, the reaction was heated at 60° C. overnight (16 h). The reaction was filtered through Celite, concentrated under reduced pressure and loaded onto Celite. The crude reaction mixture was purified by reverse phase column chromatography (dry load, C₁₈, 40 g, 05-20% MeCN:H₂O (0.1% HCO₂H) to yield the title compound (34.0 mg, 38% yield) as a colorless amorphous solid.

### Step B: 5-Chloro-2-[2-[[(3R)-1-(3-methoxypropyl)-3-piperidyl]amino]oxazolo[4,5-b]pyridin-5-yl]-3-methyl-phenol

Following the experimental procedures outlined above (Example 95) aforementioned 5-bromo-N-[(3R)-1-(3-methoxypropyl)-3-piperidyl]oxazolo[4,5-b]pyridin-2-amine (34 mg, 0.09 mmol, 1.0 eq) and (4-chloro-2-hydroxy-6-methyl-phenyl)boronic acid (21 mg, 1.2 mmol, 1.2 eq) were reacted to afford the title compound (18.6 mg, 46% yield). LCMS m/z 431.4; 433.4 (Cl isotopes) [M+H]⁺, ESI pos.

### Example 97

### 5-Chloro-3-methyl-2-[2-[[(3R)-1-(2-hydroxypropyl)-3-piperidyl]amino]oxazolo[4,5-b]pyridin-5-yl]phenol

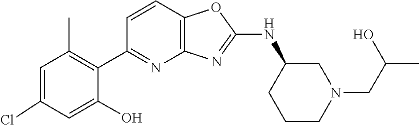

### Step A: 1-[(3R)-3-[(5-bromooxazolo[4,5-b]pyridin-2-yl)aminol-1-piperidyl]propan-2-ol

A solution of 5-bromo-N-[(3R)-3-piperidyl]oxazolo[4,5-b]pyridin-2-amine (Example 75, step B) (159 mg, 0.41 mmol, 1.0 eq) (Example 75, step 3) and potassium carbonate (281 mg, 2.0 mmol, 5.0 eq) in DMF (2 mL) was stirred for 5 mins after which a solution of 1-chloro-2-propanol (76.9 mg, 0.81 mmol, 2.0 eq) in DMF (0.5 mL) was added. The reaction mixture was heated at 60° C. for 16 hours. Then, the reaction mixture was heated up to 90° C. and stirring was continued for 5 hours. Finally, the reaction mixture was heated up to 110° C. for 16 hrs. Afterwards, the reaction mixture was allowed to cool to r.t. and left at this temperature for 5 days. The reaction mixture was diluted with water (10 mL) and 1M aq. HCl added until pH 7 was reached. EtOAc (10 mL) was added and the layers separated. The aqueous layer was extracted with EtOAc (2×10 mL) after which the combined organics were concentrated in vacuo. The crude product was purified by chromatography (silica gel, column, 0-10% (MeOH/DCM)), after which the column was flushed with 20% (0.7M NH₃ in MeOH)/DCM to yield the title compound (21.0 mg, 18% yield) as a brown oil. LCMS m/z: 355.2; 357.1 (Br isotopes) [M+H]⁺, ESI pos.

### Step B: 5-Chloro-3-methyl-2-[2-[[(3R)-1-(2-hydroxypropyl)-3-piperidyl]amino]oxazolo[4,5-b]pyridin-5-yl]phenol

Following the experimental procedures outlined above (Example 95) aforementioned 1-[(3R)-3-[(5-bromooxazolo[4,5-b]pyridin-2-yl)amino]-1-piperidyl]propan-2-ol (21.0 mg, 0.05 mmol, 1.0 eq) and (4-chloro-2-hydroxy-6-methyl-phenyl)boronic acid (11.6 mg, 0.06 mmol, 1.2 eq) were reacted to afford the title compound (8.0 mg, 33% yield). LCMS m/z 417.3 [M+H]⁺, ESI pos.

### Example 98

### 5-Chloro-2-[2-[2-hydroxyethyl-[(3R)-1-methyl-3-piperidyl]amino]oxazolo[4,5-b]pyridin-5-yl]-3-methyl-phenol

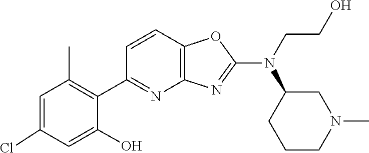 [SMILES: Cc1cc(Cl)cc(O)c1-c1ccc2oc(N(CCO)[C@@H]3CCCN(C)C3)nc2n1]

### Step A: 5-Bromo-N-[2-[tert-butyl(dimethyl)silyl]oxyethyl]-N-[(3R)-1-methyl-3-piperidyl]-oxazolo[4,5-b]pyridin-2-amine

A solution of 5-bromo-N-[(3R)-1-methyl-3-piperidyl]oxazolo[4,5-b]pyridin-2-amine (Example 30, step H) (120 mg, 0.39 mmol, 1.0 eq) in DMF (2 mL) was cooled to 0° C. and sodium hydride (60% in mineral oil, 23.1 mg, 0.58 mmol, 1.5 eq) was added. The mixture was allowed to warm to rt and stirred for 5 mins. Then (2-bromoethoxy)-tert-butyldimethylsilane (CAS #86864-60-0, 0.1 mL, 0.46 mmol, 1.2 eq) was added and stirred at rt for 16 h. The reaction mixture was cooled to 0° C. and sodium hydride (60% in mineral oil, 7.7 mg, 0.19 mmol, 0.5 eq) was added and then stirred at rt for 2 h. The reaction was quenched with water (2 mL), diluted with 10% aq. LiCl solution (10 mL) and EtOAc (3 mL) and the layers separated. The organic layer was washed with 10% aq. LiCl solution (2×10 mL) and concentrated in vacuo. The crude product was purified by chromatography (silica gel, 5-10% MeOH/DCM) to afford the title compounds (76 mg, 40% yield) as a pale-yellow solid. LCMS m/z 469.3; 471.3 (Br isotopes) [M+H]⁺, ESI pos.

### Step B: 2-[2-[2-[tert-Butyl(dimethyl)silyl]oxyethyl-[(3R)-1-methyl-3-piperidyl]amino]-oxazolo[4,5-b]pyridin-5-yl]-5-chloro-3-methyl-phenol

A solution of 5-bromo-N-[2-[tert-butyl(dimethyl)silyl]oxyethyl]-N-[(3R)-1-methyl-3-piperidyl]oxazolo[4,5-b]pyridin-2-amine (76.0 mg, 0.16 mmol, 1.0 eq), (4-chloro-2-hydroxy-6-methyl-phenyl)boronic acid (58.0 mg, 0.31 mmol, 1.92 eq) and potassium carbonate (47.0 mg, 0.34 mmol, 2.1 eq) in 1,4-dioxane (3 mL) and water (1 mL) was sparged (bubbling N₂ while sonicating) for 10 min. [1,1′-bis(diphenylphosphino)ferrocene]dichloropalladium(II) (7.0 mg, 0.01 mmol, 0.06 eq) was added and the reaction mixture was heated to 90° C. for 1 h. The reaction diluted with 50 v % brine (50 mL) and EtOAc (50 mL) and layers separated. The aqueous layer was further with extracted EtOAc (2×50 mL), the combined organic layers were dried (Na₂SO₄), filtered and concentrated under reduced pressure. The crude reaction mixture was purified by column chromatography on silica gel (40 g, 0-10% MeOH (0.7M NH₃)/EtOAc) to afford the title compound (91.0 mg, 85% yield) as a light-yellow solid. LCMS m/z 531.4 [M+H]⁺, ESI pos.

### Step C: 5-Chloro-2-[2-[2-hydroxyethyl-[(3R)-1-methyl-3-piperidyl]amino]oxazolo[4,5-b]pyridin-5-yl]-3-methyl-phenol

To a stirred solution of 2-[2-[2-[tert-butyl(dimethyl)silyl]oxyethyl-[(3R)-1-methyl-3-piperidyl]amino]oxazolo[4,5-b]pyridin-5-yl]-5-chloro-3-methyl-phenol (79.0 mg, 0.15 mmol, 1.0 eq) in 1,4-dioxane (2 mL) and methanol (200 μL) was added 4M HCl in dioxane (750.0 μL, 3.0 mmol, 20.2 eq) and the reaction mixture was stirred at rt for 45 min. The reaction was concentrated to dryness to give 68 mg of brown solid. 40 mg of the isolated material was dissolved in MeOH (˜2 mL) and applied to an SCX column. The column was washed with MeOH (50 mL), and then the product was eluted with 7M NH₃ in MeOH. The solvent was concentrated in vacuo and the resulting solid was dried in a vacuum desiccator at 45° C. to afford the title compound (28.0 mg, 43% yield) as a light brown solid. LCMS m/z 417.3 [M+H]⁺, ESI pos.

### Example 99a

### 5-Chloro-2-[2-[[(3S,4R)-1-ethyl-4-fluoro-3-piperidyl]amino]oxazolo[4,5-b]pyridin-5-yl]-3-methyl-phenol

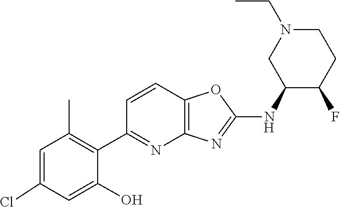

### Step A: rel cis-3-[(5-chlorooxazolo[4,5-b]pyridin-2-yl)amino]-4-fluoro-piperidine-1-carboxylic acid tert-butyl ester

Following to GP1, 5-chloro-2-(methylthio)oxazolo[4,5-b]pyridine (150 mg, 733 μmol, 1.0 eq) and rel cis-3-amino-4-fluoro-piperidine-1-carboxylic acid tert-butyl ester (201.9 mg, 879.1 μmol, 1.2 eq) were reacted in a sealed tube under MW conditions at 140° C. for 1 h, then stirring was continued for 2 d at 140° C. Afterwards, N,N-diisopropylethylamine (142.0 mg, 188 μL, 1.1 mmol, 1.5 eq) and rel cis-3-amino-4-fluoro-piperidine-1-carboxylic acid tert-butyl ester (202.0 mg, 879 μmol, 1.2 eq) were added again and the reaction was stirred at 150° C. for 1 day. After work-up and purification by flash chromatography (silica gel, gradient 0-50% EtOAc in heptane) the title compound was obtained (50.2 mg, 18%) as orange viscous oil. LCMS: m/z 371.2 [M+H]⁺, ESI pos.

### Step B: 5-Chloro-2-[2-[[(3S,4R)-1-ethyl-4-fluoro-3-piperidyl]amino]oxazolo[4,5-b]pyridin-5-yl]-3-methyl-phenol and 5-chloro-2-[2-[[(3R,4S)-1-ethyl-4-fluoro-3-piperidyl]amino]oxazolo[4,5-b]pyridin-5-yl]-3-methyl-phenol

Following to GP2a, rel cis-3-[(5-chlorooxazolo[4,5-b]pyridin-2-yl)amino]-4-fluoro-piperidine-1-carboxylic acid tert-butyl ester (175 mg, 353.95 μmol, 1.0 eq) and (4-chloro-2-hydroxy-6-methyl-phenyl)boronic acid (118.8 mg, 637 μmol, 1.8 eq) were reacted. The crude was purified by flash chromatography (gradient 0-50% EtOAc in heptane) to afford rel cis-3-[[5-(4-chloro-2-hydroxy-6-methyl-phenyl)oxazolo[4,5-b]pyridin-2-yl]amino]-4-fluoro-piperidine-1-carboxylic acid tert-butyl ester (147 mg, 80%) as yellow viscous oil. LCMS: m/z 477.3 [M+H]⁺, ESI pos. After chiral SFC separation (column chiral IK, 5 μm, 250×20 mm, 80 mL flow, 100 bar, 28% MeOH) two fractions were obtained. First eluting 5-Chloro-2-[2-[[(3S,4R)-1-ethyl-4-fluoro-3-piperidyl]amino]oxazolo[4,5-b]pyridin-5-yl]-3-methyl-phenol (Rt 3.06; 53.4 mg, 34% yield) and second eluting 5-chloro-2-[2-[[(3R,4S)-1-ethyl-4-fluoro-3-piperidyl]amino]oxazolo[4,5-b]pyridin-5-yl]-3-methyl-phenol (Rₜ 3.92; 48.5 mg, 33% yield). LCMS: 477.2 [M+H]⁺, ESI pos.

### Step C: 5-Chloro-2-[2-[[(3S,4R)-1-ethyl-4-fluoro-3-piperidyl]amino]oxazolo[4,5-b]pyridin-5-yl]-3-methyl-phenol

Aforementioned Enantiomer 5-Chloro-2-[2-[[(3S,4R)-1-ethyl-4-fluoro-3-piperidyl]amino]oxazolo[4,5-b]pyridin-5-yl]-3-methyl-phenol (53.4 mg, 104 μmol, 1.0 eq) was reacted in DCM (1 mL) and MeOH (0.5 mL) with 4 M HCl in dioxane (310 mg, 260 μL, 1.04 mmol, 10 eq). The reaction mixture was stirred at r.t. for 3 h to afford 5-chloro-2-[2-[[(3S,4R)-4-fluoro-3-piperidyl]amino]oxazolo[4,5-b]pyridin-5-yl]-3-methyl-phenol;hydrogen chloride (50.9 mg, 109%) as light yellow powder. LCMS: m/z 377.2 [M+H]⁺, ESI pos. The compound was used without further purification in the next step which was undertaken with acetaldehyde following to GP4. The title compound was obtained (38.4 mg, 84% yield) as an off-white powder. LCMS: 405.3 [M+H]⁺, ESI pos.

### Example 99b

### 5-Chloro-2-[2-[[(3R,4S)-1-ethyl-4-fluoro-3-piperidyl]amino]oxazolo[4,5-b]pyridin-5-yl]-3-methyl-phenol

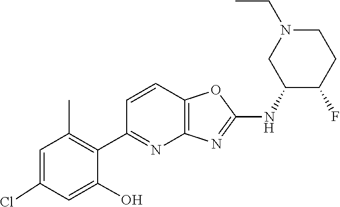

Aforementioned Enantiomer 5-chloro-2-[2-[[(3R,4S)-1-ethyl-4-fluoro-3-piperidyl]amino]oxazolo[4,5-b]pyridin-5-yl]-3-methyl-phenol (48.5 mg, 102 μmol, 1.0 eq) was reacted in DCM (1 mL) and MeOH (0.5 mL) with 4 M HCl in dioxane (254 μL, 1.02 mmol, 10 eq). The reaction mixture was stirred at r.t. for 3 h to afford 5-chloro-2-[2-[[(3R,4S)-4-fluoro-3-piperidyl]amino]oxazolo[4,5-b]pyridin-5-yl]-3-methyl-phenol;hydrogen chloride (49.6 mg, 116%) as light yellow powder. LCMS: m/z 377.2 [M+H]⁺, ESI pos. The compound was used without further purification in the next step which was undertaken with acetaldehyde following to the procedure outlined in GP4. The title compound was obtained (32.8 mg, 70% yield) as light yellow powder. LCMS: m/z 405.3 [M+H]⁺, ESI pos.

### Example 100

### (3S,5R)-5-[[5-[4-Chloro-2-hydroxy-6-(methoxymethyl)phenyl]oxazolo[4,5-b]pyridin-2-yl]amino]-1-methyl-piperidin-3-ol

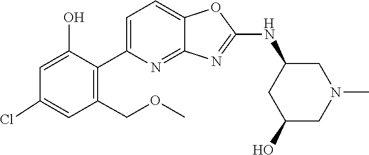

### Step A: 5-Chloro-3-hydroxy-2-iodo-benzaldehyde

To a mixture of 3-chloro-5-hydroxybenzaldehyde (CAS #1829-33-0, 10.0 g, 63.9 mmol, 1.0 eq,) in DMF (100 mL) was added NaH (3.07 g, 127.7 mmol, 2.0 eq 60%) portion wise at 0° C. under N2, stirred for 30 mins, then iodine (16.2 g, 63.9 mmol, 1.0 eq) was added portion wise and stirred for 15.5 hours at 25° C. The mixture was poured into one mole of hydrochloric acid in aqueous solution (100 mL) and extracted with ethyl acetate (100 mL×3). The combined organic layers were washed with brine (150 mL), dried over anhydrous sodium sulfate, filtered, and the filtrate was concentrated under reduced pressure. The crude was purified by column chromatography on silica gel (petroleum ether:ethyl acetate=1:0 to 5:1) and re-purified by column chromatography (C₁₈, 0.1% NH₃H₂O in water/MeCN) to afford the title compound (730 mg, 4% yield) as a light green solid. LCMS: m/z 280.9 [M−H]−, ESI neg.

### Step B: 5-Chloro-2-iodo-3-(2-trimethylsilylethoxymethoxy)benzaldehyde

A mixture of Cs₂CO₃ (1263.1 mg, 3.88 mmol, 1.5 eq) and 5-chloro-3-hydroxy-2-iodo-benzaldehyde (730.0 mg, 2.58 mmol, 1.0 eq) in DMF (10 mL) was degassed and purged with N2 three times and stirred for 30 mins. Then, SEM-Cl (646.32 mg, 3.88 mmol, 1.5 eq) was added to the mixture in one portion. The mixture was stirred at 25° C. for 2 hours. The mixture was poured into water (50 mL) and extracted with ethyl acetate (40 mL×3). The combined organic layers were washed with brine (20 mL), dried over anhydrous sodium sulfate, filtered, and the filtrate was concentrated under reduced pressure. Then, the residue was purified by column chromatography on silica gel (petroleum ether:ethyl acetate=1:0 to 10:1) to the title compound (700.0 mg, 1.7 mmol, 66% yield) as yellow oil. ¹H NMR (400 MHz, DMSO-d₆) δ [ppm]: 10.02 (s, 1H), 7.48 (d, 1H), 7.38 (d, 1H), 5.46 (s, 2H), 3.76 (t, 2H), 0.89 (t, 2H), −0.04 (s, 9H).

### Step C: [5-Chloro-2-iodo-3-(2-trimethylsilylethoxymethoxy)phenyl]methanol

Sodium borohydride (77.0 mg, 2.04 mmol, 1.20 eq) was added in small portions to a solution of 5-chloro-2-iodo-3-(2-trimethylsilylethoxymethoxy)benzaldehyde (700.0 mg, 1.7 mmol, 1.0 eq) in methanol (7 mL) and water (0.7 mL). The mixture was stirred for 3 hours at 25° C. The mixture was poured into water (15 ml) and extracted with ethyl acetate (30 mL×3). The combined organic layers were washed with brine (20 mL), dried over anhydrous sodium sulfate, filtered, and the filtrate was concentrated under reduced pressure to give the title compound (700.0 mg, 99% yield) as yellow oil. ¹H NMR (400 MHz, DMSO-d₆) δ [ppm]: 7.13 (s, 1H), 7.09 (d, 1H), 5.60 (t, 1H), 5.36 (s, 2H), 4.42-4.37 (m, 2H), 3.73 (t, 2H), 0.92-0.85 (m, 2H), −0.02-0.05 (m, 9H).

### Step D: 2-[[5-Chloro-2-iodo-3-(methoxymethyl)phenoxy]methoxy]ethyl-trimethyl-silane

To a mixture of [5-chloro-2-iodo-3-(2-trimethylsilylethoxymethoxy)phenyl]methanol (700.0 mg, 1.69 mmol, 1.0 eq) and NaH (97.22 mg, 4.05 mmol, 2.4 eq 60%) in THF (8 mL) was added Me-I (0.21 mL, 3.38 mmol, 2.0 eq) in one portion under N2 at 25° C., and stirred for 5 hours. The mixture was poured into water (30 mL) and extracted with ethyl acetate (30 mL×3). The combined organic layers were washed with brine (30 mL), dried over anhydrous sodium sulfate, filtered, and the filtrate was concentrated under reduced pressure. The residue was purified by column chromatography (SiO₂, Petroleum ether/Ethyl acetate=0/1 to 5/1) and concentrated under reduced pressure to the title compound (600.0 mg, 83% yield) as colorless oil. ¹H NMR (400 MHz, DMSO-d₆) δ [ppm]: 7.12 (d, 1H), 7.07 (d, 1H), 5.37 (s, 2H), 4.38 (s, 2H), 3.78-3.70 (m, 2H), 3.38 (s, 3H), 0.91-0.85 (m, 2H), −0.03-0.06 (m, 9H).

### Step E: 2-[[5-Chloro-2-iodo-3-(methoxymethyl)phenoxy]methoxy]ethyl-trimethyl-silane

A mixture of 2-[[5-chloro-2-iodo-3-(methoxymethyl) phenoxy]methoxy] ethyl-trimethyl-silane (60.0 mg, 0.14 mmol, 1.0 eq) and bis (pinacolato) diboron (355.35 mg, 1.4 mmol, 10.0 eq) in 1,4-dioxane (150 mL) was degassed and purged with N2 three times and KOAc (68.67 mg, 0.7 mmol, 5.0 eq), Pd(dppf)Cl₂ (10.24 mg, 0.01 mmol, 0.1 eq) was added to the mixture in one portion. The mixture was heated under microwave irradiation at 90° C. for 2 hours. The mixture was purified by C18 column chromatography (20 g, 0.1% TFA in water/MeCN to afford the title compound (20.0 mg, 0.05 mmol, 33% yield) as dark brown oil. ¹H NMR (400 MHz, CD₃OD) δ [ppm]: 7.02 (d, 1H), 6.92 (d, 1H), 5.21 (s, 2H), 4.43 (s, 2H), 3.77 (t, 2H), 3.29 (s, 3H), 1.37 (s, 12H), 0.96-0.90 (m, 3H), 0.005 (s, 9H).

### Step F: tert-Butyl (3R,5S)-3-((5-bromooxazolo[4,5-b]pyridin-2-yl)amino)-5-hydroxy-piperidine-1-carboxylate

To a solution of (3R,5S)-3-amino-5-hydroxy-piperidine-1-carboxylic acid tert-butyl ester (CAS #1932513-59-1, 277.94 mg, 1.29 mmol, 2.5 eq) and DIEA (331.6 mg, 2.57 mmol, 5.0 eq) in NMP (1 mL) was added 5-bromo-2-chloro-oxazolo[4,5-b]pyridine (120.0 mg, 0.51 mmol, 1.0 eq), stirred for 2 hours at 25° C. The mixture was filtered and the filtrate was concentrated in vacuum to give the crude product which was purified by reversed phase flash (water (0.1% TFA)-MeCN) to give the title compound (200 mg, 94% yield) as a yellow solid. ¹H NMR (400 MHz, CD₃OD) δ [ppm]: 7.43 (d, 1H), 7.09 (d, 1H), 3.93-3.83 (m, 0.3H), 3.82-3.77 (m, 1H), 3.76-7.75 (m, 0.6H), 3.72-3.55 (m, 2H), 3.41-3.29 (m, 0.5H), 3.18-3.06 (m, 0.5H), 3.04-2.87 (m, 1H), 0.2.5-2.17 (m, 1H), 1.67-1.50 (m, 1H), 1.37-1.15 (m, 9H).

### Step G: (3S,5R)-5-[(5-Bromooxazolo[4,5-b]pyridin-2-yl)amino]piperidin-3-ol;hydrochloride

A solution of tert-butyl (3R,5S)-3-[(5-bromooxazolo[4,5-b]pyridin-2-yl)amino]-5-hydroxy-piperidine-1-carboxylate (200.0 mg, 0.48 mmol, 1.0 eq) and HCl/dioxane (2.0 mL, 8.0 mmol, 16.53 eq) was stirred for 2 hrs at 25° C. The mixture was concentrated in vacuum to give the crude product which was purified by reversed phase flash (water (0.1% HCl)-MeCN) to give the title compound (160 mg, 95% yield) as a yellow solid. ¹H NMR (400 MHz, CD₃OD) δ [ppm]: 7.59 (d, 1H), 7.25 (d, 1H), 4.30-4.21 (m, 1H), 4.16-4.11 (m, 1H), 3.53-3.38 (m, 1H), 3.38-3.37 (m, 0.5H), 3.33-3.32 (m, 0.5H), 3.26-3.23 (m, 1H), 3.06-3.01 (m, 1H), 2.35-2.30 (m, 1H), 1.97-1.90 (m, 1H).

### Step H: (3S,5R)-5-[(5-Bromooxazolo[4,5-b]pyridin-2-yl)amino]-1-methyl-piperidin-3-ol;2,2,2-trifluoroacetic acid

To a solution of (3S,5R)-5-[(5-bromooxazolo[4,5-b]pyridin-2-yl)amino]piperidin-3-ol;hydrochloride (150.0 mg, 0.43 mmol, 1.0 eq), NaOAc (42.23 mg, 0.51 mmol, 1.2 eq), in methanol (2 mL) was added formaldehyde (104.45 mg, 1.29 mmol, 3.0 eq, 37%) under N2, and stirred at 25° C. for 0.5 h, then NaBH₃CN (134.81 mg, 2.15 mmol, 5.0 eq) was added and stirring was continued for 1.5 hours at 25° C. The mixture was filtered and the filtrate was concentrated in vacuum. The crude was purified by reversed phase flash (water (0.1% TFA)-MeCN) to the title compound (130.0 mg, 67% yield) as a yellow solid. ¹H NMR (400 MHz, CD₃OD) δ [ppm]: 7.58 (d, 1H), 7.24 (d, 1H), 4.47-4.13 (m, 2H), 3.57-3.31 (m, 4H), 2.94 (s, 3H), 2.43-2.00 (m, 2H).

### Step I: (3S,5R)-5-[[5-[4-Chloro-2-(methoxymethyl)-6-(2-trimethylsilylethoxymethoxy)-phenyl]oxazolo[4,5-b]pyridin-2-yl]amino]-1-methyl-piperidin-3-ol

A mixture of K₂CO₃ (16.92 mg, 0.12 mmol, 2.5 eq), 2-[[5-chloro-3-(methoxymethyl)-2-(4,4,5,5-tetramethyl-1,3,2-dioxaborolan-2-yl)phenoxy]methoxy]ethyl-trimethyl-silane (21.0 mg, 0.05 mmol, 1.0 eq) and (3S,5R)-5-[(5-bromooxazolo[4,5-b]pyridin-2-yl)amino]-1-methyl-piperidin-3-ol;2,2,2-trifluoroacetic acid (28.1 mg, 0.06 mmol, 1.3 eq) in 1,4-dioxane (1 mL) and water (0.200 mL) was degassed and purged with N2 three times and XPhos Pd G3 (4.12 mg, 0.0 mmol, 0.1 eq) was added to the mixture. The mixture was heated under microwave irradiation at 95° C. for 2 h. The mixture was concentrated under reduced pressure and purified by C18 column chromatography (20 g, 0.1% NH₃H₂O in water/MeCN) to afford the title compound (20.0 mg, 74% yield) as a red oil. ¹H NMR (400 MHz, CD₃OD) δ [ppm]: 7.80 (d, 1H), 7.24 (d, 1H), 7.21 (s, 1H), 7.08 (d, 1H), 5.13 (s, 2H), 4.76-4.58 (m, 1H), 4.49-4.32 (m, 1H), 4.21 (s, 2H), 3.95-3.75 (m, 1H), 3.59 (t, 2H), 3.53-3.37 (m, 1H), 3.26 (s, 1H), 3.21 (s, 3H), 2.94 (s, 3H), 2.89-2.66 (m, 1H), 2.60-2.43 (m, 1H), 2.30-2.03 (m, 2H), 1.79-1.53 (m, 1H), 0.86 (t, 2H), −0.04 (s, 9H).

### Step J: (3S,5R)-5-[[5-[4-Chloro-2-hydroxy-6-(methoxymethyl)phenyl]oxazolo[4,5-b]pyridin-2-yl]amino]-1-methyl-piperidin-3-ol

A solution of (3S,5R)-5-[[5-[4-chloro-2-(methoxymethyl)-6-(2-trimethylsilylethoxymethoxy) phenyl]oxazolo[4,5-b]pyridin-2-yl]amino]-1-methyl-piperidin-3-ol (20.0 mg, 0.04 mmol, 1.0 eq) and TFA (0.5 mL, 6.73 mmol, 184.82 eq) in DCM (1 mL) was stirred at 25° C. for 2 h. The residue was purified by C18 column chromatography (20 g, 0.1% TFA in water/MeCN) to afford the title compound (8.63 mg, 54% yield) as a yellow solid. ¹H NMR (400 MHz, CD₃OD) δ [ppm]: 7.90 (d, 1H), 7.20 (d, 1H), 7.04 (d, 1H), 6.92 (d, 1H), 4.80-4.34 (m, 2H), 4.24 (s, 2H), 4.06-3.79 (m, 1H), 3.69-3.36 (m, 2H), 3.21 (s, 3H), 2.95 (s, 3H), 2.85-2.66 (m, 1H), 2.26-2.07 (m, 1H), 2.03 (s, 1H), 1.94-1.49 (m, 1H).

### Example 101

### 1-[2-[[(3R)-1-Ethyl-3-piperidyl]amino]-5-[2-hydroxy-6-methyl-4-(trifluoromethyl)phenyl]-oxazolo[4,5-b]pyridin-7-yl]azetidin-3-ol

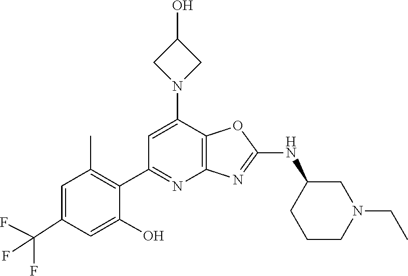

### Step A: 2-[7-Chloro-2-[[(3R)-1-ethyl-3-piperidyl]amino]oxazolo[4,5-b]pyridin-5-yl]-3-methyl-5-(trifluoromethyl)phenol

tert-Butyl (3R)-3-[[7-chloro-5-[2-methoxy-6-methyl-4-(trifluoromethyl)phenyl]oxazolo[4,5-b]pyridin-2-yl]amino]piperidine-1-carboxylate (275.0 mg, 0.46 mmol, 1.0 eq) was dissolved in DCM (15 mL) and boron tribromide (1 M in DCM) (2.29 mL, 2.29 mmol, 5.0 eq) was added. The mixture was stirred at room temperature for 2 h, and then concentrated in vacuo. The resulting residue was dissolved in 0.7 N ammonia in MeOH (5 mL), then concentrated in vacuo. The resulting residue was dissolved in EtOH (10 mL) and one drop of AcOH was added, followed by acetaldehyde (0.1 mL, 1.83 mmol, 4.0 eq). The reaction was stirred for 1 h, then STAB (242.42 mg, 1.14 mmol, 2.5 eq) was added. The reaction was stirred for 1 h, then wet MeOH (5 mL) and the mixture stirred at room temperature for 45 min. The reaction mixture was dry-loaded onto silica and purified by flash chromatography on silica gel (12 g column, 0-10% (0.7 N ammonia in MeOH/DCM)) to afford the title compound (195 mg, 93% yield) as a light brown solid. LCMS m/z 455.2 [M+H]⁺, ESI pos.

### Step B: 1-[2-[[(3R)-1-Ethyl-3-piperidyl]amino]-5-[2-hydroxy-6-methyl-4-(trifluoromethyl)-phenyl]oxazolo[4,5-b]pyridin-7-yl]azetidin-3-ol

2-[7-chloro-2-[[(3R)-1-ethyl-3-piperidyl]amino]oxazolo[4,5-b]pyridin-5-yl]-3-methyl-5-(trifluoromethyl)phenol (Example 101, step A) (150 mg, 0.33 mmol, 1.0 eq) was dissolved in NMP (2 mL) and 3-hydroxyazetidine (482.1 mg, 6.6 mmol, 20.0 eq) was added. The mixture was sealed in a tube and stirred at 80° C. for 24 h. The reaction mixture was diluted to 5.1 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% Ammonia in water-MeCN gradient over 12.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 25% MeCN; 0.5-10.5 min, ramped from 25% MeCN to 55% MeCN; 10.5-10.6 min, ramped from 55% MeCN to 100% MeCN; 10.6-12.5 min, held at 100% MeCN. This afforded the title compound (6.35 mg, 3.7% yield) as a white solid. LCMS m/z 492.3 [M+H]⁺, ESI pos.

### Example 102

### 1-[5-[2-Hydroxy-6-methyl-4-(trifluoromethyl)phenyl]-2-[[(3R)-1-methyl-3-piperidyl]amino]oxazolo[4,5-b]pyridin-7-yl]ethanone;2,2,2-trifluoroacetic acid

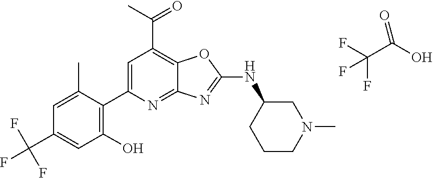

### Step A: 6-Chloro-4-iodo-2-nitropyridin-3-ol

The apparatus was dried by heating with a heatgun under vacuum. In a three round bottom flask, 6-chloro-4-iodo-pyridin-3-ol (CAS: 877133-58-9; 5.0 g, 19.57 mmol, 1.0 eq) in acetic acid (50 mL) was added fuming nitric acid (2.45 mL, 58.72 mmol, 3.0 eq) at 20° C., then the mixture was stirred at 20° C. for 2 hours. The reaction mixture was cooled to r.t. It was slowly pipetted into 100 mL of sat. aq. NaHCO₃ solution. Gas evolution was observed. EtOAc (120 mL) and water (120 mL) were added and layers were separated. The aqueous phase was extracted with EtOAc (100 mL×2). Combined extracts were washed with brine (100 mL), dried over Na₂SO₄, filtered, and concentrated under vacuum to give a yellow solid. The solid was purified by column (PE:EA=4:1 to 2:1) to give the title compound (2.7 g, 46% yield) as yellow solid. LCMS: m/z 301.0 [M+H]⁺, ESI pos.

### Step B: 2-Amino-6-chloro-4-iodopyridin-3-ol

To a mixture of 6-chloro-4-iodo-2-nitro-pyridin-3-ol (2.8 g, 9.32 mmol, 1.0 eq) in methanol (28 mL) was added tin(II) chloride (8.84 g, 46.6 mmol, 5.0 eq), and the mixture was stirred at 70° C. for 2 hours. The reaction mixture was cooled to r.t. EtOAc (80 mL) and water (80 mL) were added and layers were separated. The aqueous phase was extracted with EtOAc (60 mL×2). Combined extracts were washed with brine (80 mL), dried over Na₂SO₄, filtered, and concentrated under vacuum to give a yellow solid. The residue was purified by column (PE:EA=5:1 to 1:1) to give the title compound (1.7 g, 67% yield) as a yellow solid. LCMS: m/z 270.9 [M+H]⁺, ESI pos.

### Step C: 5-Chloro-7-iodooxazolo[4,5-b]pyridine-2-thiol

The apparatus was dried by heating with a heatgun under vacuum. To a mixture of 2-amino-6-chloro-4-iodo-pyridin-3-ol (2.3 g, 8.5 mmol, 1.0 eq) in THF (23 mL) was added TCDI (3.03 g, 17.01 mmol, 2.0 eq), and the mixture was stirred at 50° C. for 12 hours. The reaction mixture was cooled to r.t. EtOAc (30 mL) and water (40 mL) were added and layers were separated. The aqueous phase was extracted with EtOAc (40 mL×2). Combined extracts were washed with brine (40 mL), dried over Na₂SO₄, filtered, and concentrated under vacuum to give a brown oil. The residue was purified by column (PE:EA=10:1 to 5:1) to give the title compound (1.3 g, 49% yield) as a brown oil. LCMS: m/z 313.0 [M+H⁺, ESI pos.

### Step D: 2,5-Dichloro-7-iodooxazolo[4,5-b]pyridine

To a mixture of 5-chloro-7-iodo-7,7a-dihydrooxazolo[4,5-b]pyridine-2-thiol (900 mg, 2.86 mmol, 1.0 eq) in oxalyl chloride (12.1 mL, 143.07 mmol, 50.0 eq) was added DMF (20.9 mg, 0.29 mmol, 0.1 eq), then the mixture was stirred at 50° C. for 1 h. The reaction mixture was cooled to r.t. EtOAc (20 mL) and water (20 mL) were added and layers were separated. The aqueous phase was extracted with EtOAc (20 mL×2). Combined extracts were washed with brine (20 mL), dried over Na₂SO₄, filtered, and concentrated under vacuum to give a yellow solid. The solid was purified by column (PE:EA=5:1 to 2:1) to give the title compound (360.0 mg, 1.14 mmol, 40% yield) as a yellow solid. LCMS: m/z 314.9 [M+H]⁺, ESI pos.

### Step E: (R)-5-Chloro-7-iodo-N-(1-methylpiperidin-3-yl)oxazolo[4,5-b]pyridin-2-amine 2,2,2-trifluoroacetate

To a mixture of (3R)-1-methylpiperidin-3-amine (377.1 mg, 3.3 mmol, 2.0 eq) in MeCN (5 mL) was added 2,5-dichloro-7-iodo-oxazolo[4,5-b]pyridine (520.0 mg, 1.65 mmol, 1.0 eq) and DIEA (0.54 mL, 3.3 mmol, 2.0 eq), then the mixture was stirred at 25° C. for 0.5 hours. The reaction mixture was quenched by addition of 1 ml water, then diluted with 3 ml MeOH to get a brown mixture. The mixture was purified by reversed phase flash (0.1% TFA water/ACN condition). The eluate was dried by lyolization to get a yellow solid. The solid was re-purified by prep-HPLC (Column: 3_Phenomenex Luna C18 75*30 mm*3 um; Condition: water(TFA)-ACN; Begin B: 15; End B: 35; Gradient Time(min): 9; 100% B Hold Time(min): 2; FlowRate (ml/min): 25) to yield the title compound (325 mg, 50% yield) as a yellow solid. LCMS: m/z 393.1 [M+H]⁺, ESI pos.

### Step F: (R)-1-(5-Chloro-2-((1-methylpiperidin-3-yl)amino)oxazolo[4,5-b]pyridin-7-yl)ethan-1-one

5-Chloro-7-iodo-N-[(3R)-1-methyl-3-piperidyl]oxazolo[4,5-b]pyridin-2-amine (100.0 mg, 0.25 mmol, 1.0 eq) and tributyl(1-ethoxyvinyl)stannane (0.44 g, 1.21 mmol, 4.77 eq) in DMF (3 mL) was degassed with nitrogen for 5 minutes. Pd(PPh₃)₄ (58.78 mg, 0.05 mmol, 0.2 eq) was added, and the mixture was heated to 80° C. for 6 hours. The reaction mixture was cooled to r.t. EtOAc (20 mL) and water (20 mL) were added and layers were separated. The aqueous phase was extracted with EtOAc (30 mL×2). Combined extracts were washed with brine (30 mL), dried over Na₂SO₄, filtered, and concentrated under vacuum to give a white solid. The residue was purified by column (PE:EA=20:1 to 10:1) to give the title compound (40.0 mg, 51% yielded) as a white solid LCMS: m/z 309.2 [M+H]+, ESI pos.

### Step G: 1-[5-[2-Hydroxy-6-methyl-4-(trifluoromethyl)phenyl]-2-[[(3R)-1-methyl-3-piperidyl]amino]oxazolo[4,5-b]pyridin-7-yl]ethanone;2,2,2-trifluoroacetic acid

To a solution of 1-[5-chloro-2-[[(3R)-1-methyl-3-piperidyl]amino]oxazolo[4,5-b]pyridin-7-yl]ethanone (10.0 mg, 0.03 mmol, 1.0 eq) and [2-hydroxy-6-methyl-4-(trifluoromethyl)phenyl]boronic acid (28.49 mg, 0.13 mmol, 4.0 eq) in 1,4-dioxane (1 mL) and water (0.2 mL) was added CsF (19.7 mg, 0.13 mmol, 4.0 eq) and XPhos Pd G3 (2.74 mg, 0.0 mmol, 0.1 eq) under nitrogen atmosphere. The mixture was stirred at 95° C. for 5 hours. The reaction mixture was cooled to r.t. and concentrated in vacuum. The crude was purified by C18 column chromatography (20 g, 0.1% TFA in water/MeCN) to give the title compound (5.03 mg, 34% yield) as a white solid. LCMS: m/z 449.2 [M+H]+, ESI pos.

### Example 103

### 2-[2-[[(3R)-1-Ethyl-3-piperidyl]amino]-7-(methylamino)oxazolo[4,5-b]pyridin-5-yl]-3-methyl-5-(trifluoromethyl)phenol

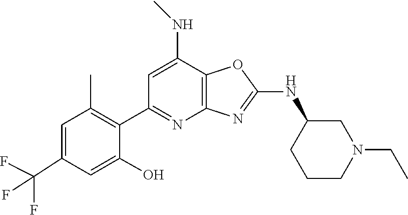

### Step A: tert-Butyl (3R)-3-[[5-[2-methoxy-6-methyl-4-(trifluoromethyl)phenyl]oxazolo[4,5-b]pyridin-2-yl]amino]piperidine-1-carboxylate

2-[2-Methoxy-6-methyl-4-(trifluoromethyl)phenyl]-4,4,5,5-tetramethyl-1,3,2-dioxaborolane (725.71 mg, 2.3 mmol, 1.2 eq), XPhos Pd G3 (121.59 mg, 0.14 mmol, 0.08 eq), sat. aq. NaHCO₃ (1.75 mL) and tert-butyl-(3R)-3-[(5-bromooxazolo[4,5-b]pyridin-2-yl)amino]piperidine-1-carboxylate (Example 75, Step A) (760 mg, 1.91 mmol, 1.0 eq) were suspended in 1,4-dioxane (12 mL) and degassed with N₂ (5 min). The reaction mixture was heated to 80° C. and stirred for 90 min. The reaction mixture was cooled, dry-loaded onto silica gel and purified by chromatography (silica gel, 0-10% MeOH/DCM) to afford the title compound (824 mg, 71% yield) as a yellow solid. LCMS m/z 541.4 (³⁵Cl) [M+H]+, ESI pos.

### Step B: tert-Butyl (3R)-3-[[5-[2-methoxy-6-methyl-4-(trifluoromethyl)phenyl]-4-oxido-oxazolo[4,5-b]pyridin-4-ium-2-yl]amino]piperidine-1-carboxylate

Methyltrioxorhenium(VII) (79.7 mg, 0.32 mmol, 0.2 eq) was added to a stirred solution of 30% aq. hydrogen peroxide (0.82 mL, 8.0 mmol, 5.0 eq) and tert-butyl (3R)-3-[[5-[2-methoxy-6-methyl-4-(trifluoromethyl)phenyl]oxazolo[4,5-b]pyridin-2-yl]amino]piperidine-1-carboxylate (900.0 mg, 1.6 mmol, 1.0 eq) in MeOH (12 mL). The reaction mixture was stirred at room temperature for 2 h and then further methyltrioxorhenium(vii) (83.7 mg, 0.34 mmol, 0.2 eq) was added and stirred at RT for 24 hr. The reaction mixture was added slowly to a stirred 10 wt % aqueous Na₂SO₃ (30 mL) solution at 0° C. The mixture was diluted with water (50 mL) and extracted with EtOAc (2×50 mL). The combined organic extracts were dried using a phase separator and concentrated in vacuo. The resulting residue was purified by flash chromatography (silica gel, 0-10% MeOH/DCM) to afford the title compound (600 mg, 62%) as a light yellow solid. LCMS m/z 523.4 [M+H]⁺, ESI pos.

### Step C: tert-Butyl (3R)-3-[[7-chloro-5-[2-methoxy-6-methyl-4-(trifluoromethyl)-phenyl]oxazolo[4,5-b]pyridin-2-yl]amino]piperidine-1-carboxylate

tert-Butyl (3R)-3-[[5-[2-methoxy-6-methyl-4-(trifluoromethyl)phenyl]-4-oxido-oxazolo[4,5-b]pyridin-4-ium-2-yl]amino]piperidine-1-carboxylate (Example 103, step B) (420 mg, 0.8 mmol, 1.0 eq) was dissolved in DMF (12 mL) and oxalyl chloride (0.41 mL, 4.82 mmol, 6.0 eq) was added slowly with vigorous stirring at 0° C. The mixture was stirred at room temperature for 2 h, then 2 N aqueous NaOH (2 mL) was added, and the mixture stirred for a further 45 min. The mixture was diluted with EtOAc (30 mL), washed with 10 wt % aqueous LiCl (3×25 mL), dried using a phase separator and concentrated in vacuo. The resulting residue was purified by chromatography on silica gel (12 g column, 0-10% MeOH/DCM) to afford the title compound (300 mg, 62% yield) as a light brown gum. LCMS m/z 541.4 (³⁵Cl) [M+H]+, ESI pos.

### Step D: tert-Butyl (3R)-3-[[7-[tert-butoxycarbonyl(methyl)amino]-5-[2-methoxy-6-methyl-4-(trifluoromethyl)phenyl]oxazolo[4,5-b]pyridin-2-yl]amino]piperidine-1-carboxylate

tert-Butyl N-methylcarbamate (270.6 mg, 2.06 mmol, 10.0 eq), XPhos Pd G3 (52.39 mg, 0.06 mmol, 0.3 eq), XPhos (29.5 mg, 0.06 mmol, 0.3 eq), tert-butyl (3R)-3-[[7-chloro-5-[2-methoxy-6-methyl-4-(trifluoromethyl)phenyl]oxazolo[4,5-b]pyridin-2-yl]amino]piperidine-1-carboxylate (120.0 mg, 0.21 mmol, 1.0 eq) and Cs₂CO₃ (336.1 mg, 1.03 mmol, 5.0 eq) were suspended in 1,4-dioxane (2 mL) and the mixture degassed (N2, 5 min). The mixture was stirred at 80° C. for 4 h. The mixture was cooled and further XPhos (29.5 mg, 0.06 mmol, 0.3 eq) and XPhosPdG3 (52.39 mg, 0.06 mmol, 0.3 eq) were added. The mixture was placed under nitrogen and stirred at 80° C. for a further 16 h. The reaction mixture cooled, diluted with water (50 mL) and extracted with EtOAc (2×30 mL). The combined organic extracts were dried using a phase separator and concentrated in vacuo. The resulting residue was purified by flash chromatography (silica gel, 0-10% MeOH/DCM) to afford the title compound (76.0 mg, 32% yield) as a yellow oil. LCMS m/z 636.5 [M+H]+, ESI pos.

### Step E: 2-[2-[[(3R)-1-Ethyl-3-piperidyl]amino]-7-(methylamino)oxazolo[4,5-b]pyridin-5-yl]-3-methyl-5-(trifluoromethyl)phenol

tert-Butyl (3R)-3-[[7-[tert-butoxycarbonyl(methyl)amino]-5-[2-methoxy-6-methyl-4-(trifluoro-methyl)phenyl]oxazolo[4,5-b]pyridin-2-yl]amino]piperidine-1-carboxylate (76.0 mg, 0.07 mmol, 1.0 eq) was dissolved in DCM (4 mL) and boron tribromide (1 M in DCM) (0.33 mL, 0.33 mmol, 5.0 eq) was added. The mixture was stirred at r.t. for 16 h, then concentrated in vacuo. The resulting residue was dissolved in 0.7 N ammonia in MeOH (5 mL), then concentrated in vacuo. The resulting residue was dissolved in EtOH (4 mL) and one drop of AcOH was added, followed by acetaldehyde (0.02 mL, 0.27 mmol, 4.0 eq). The reaction was stirred for 1 h, then STAB (56.76 mg, 0.27 mmol, 4.0 eq) was added. The reaction was stirred for 1 h, then wet MeOH (5 mL) was added, and the mixture stirred for 30 min. The reaction mixture was dry-loaded onto silica and purified by flash chromatography on silica gel (4 g column, 0-20% (0.7 N ammonia in MeOH)/DCM) to afford the title compound (17.4 mg, 50% yield) as a light brown solid. LCMS: m/z 450.4 [M+H]+, ESI pos.

### Example 104

### 2-[2-[[(3R)-1-Ethyl-3-piperidyl]amino]-7-methoxy-oxazolo[4,5-b]pyridin-5-yl]-3-methyl-5-(trifluoromethyl)phenol

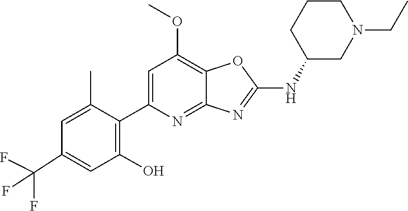

### Step A: 6-Bromo-4-methoxy-2-nitro-pyridin-3-ol

To a magnetically stirred solution of 4,6-dibromo-2-nitro-pyridin-3-ol (450 mg, 1.5 mmol, 1.0 eq) in DMSO (12 mL) was added sodium methoxide (81.6 mg, 1.51 mmol, 1.00 eq) at room temperature. The reaction mixture was heated at 60° C. for 30 minutes, then additional sodium methoxide (1.5 eq) were added and stirring was continued for 6 hours. The reaction mixture was cooled to 15-20° C. and quenched with 1.5 N HCl. The aq. layer (pH˜ 1) was extracted twice with MTBE. The combined organic layers were concentrated and the crude was purified by normal phase chromatography (DCN-MeOH 0 to 10%) to the title compound (239 mg, 60% yield) as yellow solid. LCMS m/z: 251 [M+H]⁺, ESI pos.

### Step B: 6-[2-Hydroxy-6-methyl-4-(trifluoromethyl)phenyl]-4-methoxy-2-nitro-pyridin-3-ol

A mixture of 6-bromo-4-methoxy-2-nitro-pyridin-3-ol (55 mg, 210 μmol, 1.0 eq), 3-methyl-2-(4,4,5,5-tetramethyl-1,3,2-dioxaborolan-2-yl)-5-(trifluoromethyl)phenol (100 mg, 315 μmol, 1.5 eq), cesium carbonate (136.7 mg, 420 μmol, 2.0 eq) and XPhos Pd G3 (18.7 mg, 22.0 μmol, 0.105 eq) in 1,4-dioxane (1 mL) and water (0.25 mL) was flushed with argon and stirred at 90° C. overnight. The mixture was cooled to ambient temperature, filtered over decalite and the solvent was removed under reduced pressure. The crude was purified by preparative HPLC (column: YMC-Triart C₁₈, 12 nm, 5 μm, 100×30 mm; gradient: MeCN/water+0.1% HCOOH) to yield the title compound (17 mg, 23% yield). ¹H NMR (300 MHz, CDCl₃) δ [ppm]: 2.54 (s, 3H) 4.08 (s, 3H) 7.10 (s, 1H) 7.16-7.19 (m, 1H) 7.33 (s, 1H) 10.10 (s, 1H) 10.31-10.52 (m, 1H). The reaction was repeated on larger scale.

### Step C: 2-Amino-6-[2-hydroxy-6-methyl-4-(trifluoromethyl)phenyl]-4-methoxy-pyridin-3-ol

6-[2-Hydroxy-6-methyl-4-(trifluoromethyl)phenyl]-4-methoxy-2-nitro-pyridin-3-ol (122 mg, 354 μmol, 1.0 eq) was dissolved in methanol (12.2 mL) and Pd/C (12.2 mg, 11.46 μmol, 0.032 eq) was added at rt. The mixture was degassed 3 times and was then stirred under H₂ for 1 hour. The reaction mixture was filtered over decalite, rinsed with MeOH and evaporated to dryness to give the title compound (104.9 mg, 91%) as dark green powder. The product was directly used in the next step without any further purification. LCMS m/z: 315.2 [M+H]⁺, ESI pos.

### Step E: 2-[7-Methoxy-2-(methylthio)oxazolo[4,5-b]pyridin-5-yl]-3-methyl-5-(trifluoro-methyl)phenol

To a solution of 2-amino-6-[2-hydroxy-6-methyl-4-(trifluoromethyl)phenyl]-4-methoxy-pyridin-3-ol (104 mg, 331 μmol, 1.0 eq) in DMF (1.1 mL) under argon atmosphere was added 1,1′-thiocarbonyldiimidazole (59 mg, 331 μmol, 1.0 eq). The reaction mixture was stirred at r.t. overnight. Afterwards, K₂CO₃ (91.5 mg, 662 μmol, 2.0 eq) was added followed by methyl iodide (54.5 mg, 24 μL, 384 μmol, 1.16 eq). The reaction mixture was stirred at r.t. for 30 min. After cooling to r.t., the mixture was then extracted with ammonium chloride, water and ethyl acetate. The aqueous layers were back-extracted with ethyl acetate. The combined organic layers were washed with brine, dried over anhydrous sodium sulfate, filtered and concentrated in vacuo. The crude was purified by flash chromatography (gradient 0-20% ethyl acetate in heptane) to afford the title compound (98.5 mg, 79% yield) as a white foam. LCMS m/z: 371.1 [M+H]⁺, ESI pos.

### Step F: 2-[2-[[(3R)-1-Ethyl-3-piperidyl]aminol-7-methoxy-oxazolo[4,5-b]pyridin-5-yl]-3-methyl-5-(trifluoromethyl)phenol

To a colorless mixture of 2-[7-methoxy-2-(methylthio)oxazolo[4,5-b]pyridin-5-yl]-3-methyl-5-(trifluoromethyl)phenol (32.6 mg, 86 μmol, 1.0 eq) in 1,4-dioxane (400 μL) was added under stirring [(3R)-1-ethyl-3-piperidyl]amine (13.0 mg, 102 μmol, 1.18 eq) followed by triethylamine (10.1 mg, 13.9 μL, 100 μmol, 1.16 eq) at r.t. The solution was stirred at 90° C. for 3 days. Then, NEt₃ (10.1 mg, 13.9 μL, 100 μmol, 1.16 eq) and [(3R)-one-ethyl-3-piperidyl] amine (13 mg, 102 μmol, 1.18 eq) was added and stirring was continued at 90° C. overnight. After cooling to r.t., the mixture was extracted with EtOAc, water and ammonium chloride. The aqueous layers were back-extracted with ethyl acetate twice. The combined organic layers were washed with water and brine, then dried over anhydrous sodium sulfate, filtered and concentrated in vacuo. The crude was purified by preparative HPLC (column: Gemini NX, 12 nm, 5 μm, 100×30 mm, gradient: MeCN/water+0.10% TEA) to afford the title compound (13.1 mg, 30% yield) as a white solid. LCMS m/z: 451.2 [M+H]⁺, ESI pos.

### Example 105 and 106

### 2-[7-(1-Hydroxy-1-methyl-ethyl)-2-[[(3R)-1-methyl-3-piperidyl]amino]oxazolo[4,5-b]pyridin-5-yl]-3-methyl-5-(trifluoromethyl)phenol;2,2,2-trifluoroacetic acid and 2-[7-(1-Hydroxyethyl)-2-[[(3R)-1-methyl-3-piperidyl]amino]oxazolo[4,5-b]pyridin-5-yl]-3-methyl-5-(trifluoromethyl)phenol

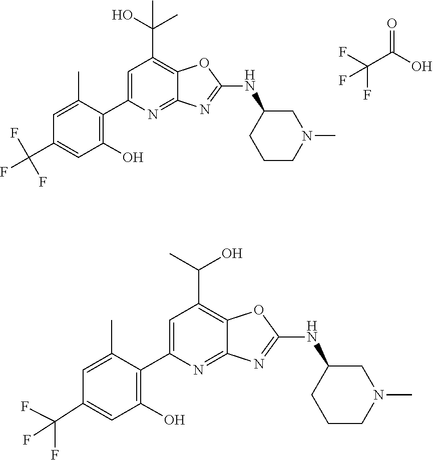

### Step A: Methyl (R)-5-chloro-2-((1-methylpiperidin-3-yl)amino)oxazolo[4,5-b]pyridine-7-carboxylate

To a solution of (R)-5-chloro-7-iodo-N-(1-methylpiperidin-3-yl)oxazolo[4,5-b]pyridin-2-amine (Example 60, step E) (90.0 mg, 0.23 mmol, 1.0 eq) and methanol (172.1 mg, 3.44 mmol, 15.0 eq) in DMF (5 mL) was added TEA (116 mg, 1.15 mmol, 5.0 eq) and Pd(dppf)Cl₂ (16.8 mg, 0.02 mmol, 0.1 eq). The mixture was degassed with nitrogen three times and stirred at 80° C. under CO atmosphere at 2280 mmHg for 3 hours. After cooling to ambient temperature, the reaction mixture was concentrated under reduced pressure. The residue was purified by column chromatography (C₁₈, 0.1% TFA in water-MeCN), to afford the title compound (60.0 mg, 81% yield) as a yellow solid. LCMS: m/z 325.1 [M+H]⁺, ESI pos.

### Step B: (R)-2-(5-Chloro-2-((1-methylpiperidin-3-yl)amino)oxazolo[4,5-b]pyridin-7-yl)propan-2-ol and (R)-1-(5-chloro-2-((1-methylpiperidin-3-yl)amino)oxazolo[4,5-b]pyridin-7-yl)ethan-1-one

To a solution of methyl (R)-5-chloro-2-((1-methylpiperidin-3-yl)amino)oxazolo[4,5-b]pyridine-7-carboxylate (40.0 mg, 0.12 mmol, 1.0 eq) in THF (5 mL) under nitrogen atmosphere, methyl lithium (0.88 mL, 1.23 mmol, 10.0 eq) was added dropwise at −65° C. and the stirring was continued for 2 hours. The mixture was quenched with water (1 mL) at −50° C. and concentrated under reduced pressure. The residue was purified by column chromatography (Cis, 0.1% TFA in water/MeCN) to afford (R)-2-(5-chloro-2-((1-methylpiperidin-3-yl)amino)oxazolo[4,5-b]pyridin-7-yl)propan-2-ol (20.0 mg, 50% yield) as yellow oil and 1-(R)-1-(5-chloro-2-((1-methylpiperidin-3-yl)amino)oxazolo[4,5-b]pyridin-7-yl)ethan-1-one (10.0 mg, 26% yield) as yellow oil. LCMS of (R)-2-(5-chloro-2-((1-methylpiperidin-3-yl)amino)oxazolo[4,5-b]pyridin-7-yl)propan-2-ol: m/z 325.4, [M+H]⁺, ESI pos.

### Step C: 2-[7-(1-Hydroxy-1-methyl-ethyl)-2-[[(3R)-1-methyl-3-piperidyl]amino]oxazolo[4,5-b]pyridin-5-yl]-3-methyl-5-(trifluoromethyl)phenol;2,2,2-trifluoroacetic acid and (R)-1-(5-(2-hydroxy-6-methyl-4-(trifluoromethyl)phenyl)-2-((1-methylpiperidin-3-yl)amino)oxazolo[4,5-b]pyridin-7-yl)ethan-1-one

The apparatus was dried by heating with a heat gun under vacuum. To a mixture of (R)-2-(5-chloro-2-((1-methylpiperidin-3-yl)amino)oxazolo[4,5-b]pyridin-7-yl)propan-2-ol (20.0 mg, 0.06 mmol, 1.0 eq), 1-(R)-1-(5-chloro-2-((1-methylpiperidin-3-yl)amino)oxazolo[4,5-b]pyridin-7-yl)ethan-1-one (9.5 mg, 0.03 mmol, 0.5 eq), CsF (46.77 mg, 0.31 mmol, 5.0 eq) and [2-hydroxy-6-methyl-4-(trifluoromethyl)phenyl]boronic acid (40.63 mg, 0.18 mmol, 3.0 eq) in 1,4-dioxane (1 mL)/water (0.2 mL) was added XPhos Pd G3 (5.22 mg, 0.01 mmol, 0.1 eq) at 25° C. The mixture was stirred at 125° C. for 2 hours in the microwave. After cooling to ambient temperature, the mixture was quenched by addition of water (10 mL), filtered, and the filtrate was concentrated under reduced pressure. The residue was purified by C18 column chromatography (20 g, 0.1% TFA in water/MeCN) to afford (R)-2-(7-(2-hydroxypropan-2-yl)-2-((1-methylpiperidin-3-yl)amino)oxazolo[4,5-b]pyridin-5-yl)-3-methyl-5-(trifluoromethyl)phenol;2,2,2-trifluoroacetic acid (16.4 mg, 0.03 mmol, 44% yield) as a light yellow solid. LCMS: m/z 465.2, (M+H)⁺ (ESI⁺); and (R)-1-(5-(2-hydroxy-6-methyl-4-(trifluoromethyl)phenyl)-2-((1-methylpiperidin-3-yl)-amino)oxazolo[4,5-b]pyridin-7-yl)ethan-1-one (5.0 mg, 18% yield) as a light yellow solid. LCMS: m/z 449.2 [M+H]⁺, ESI pos.

### Step D: 2-[7-(1-Hydroxyethyl)-2-[[(3R)-1-methyl-3-piperidyl]amino]oxazolo[4,5-b]pyridin-5-yl]-3-methyl-5-(trifluoromethyl)phenol

To a solution of (R)-1-(5-(2-hydroxy-6-methyl-4-(trifluoromethyl)phenyl)-2-((1-methylpiperidin-3-yl)amino)oxazolo[4,5-b]pyridin-7-yl)ethan-1-one (5.0 mg, 0.01 mmol, 1.0 eq) in methanol (1 mL) was added NaBH₄ (0.84 mg, 0.02 mmol, 2.0 eq), and the mixture was stirred for 0.5 hour at 25° C. After filtration, the solvents were removed under reduced pressure and the crude was purified by C₁₈ column chromatography (20 g, 0.1% NH₃H₂O in water/MeCN) to afford the title compound (2.31 mg, 43% yield) as a yellow solid. LCMS: m/z 451.1 [M+H]+, ESI pos.

### Example 107

### 3-Chloro-4-[2-[[(3R)-1-ethyl-3-piperidyl]amino]oxazolo[4,5-b]pyridin-5-yl]-5-hydroxy-benzonitrile;2,2,2-trifluoroacetic acid

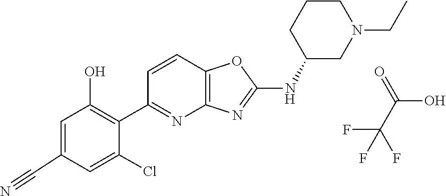

### Step A: 3-Chloro-5-hydroxy-4-iodobenzoic acid

To a mixture of 3-chloro-5-hydroxybenzoic acid (CAS: 53984-36-4; 0.205 g, 1.19 mmol, 1.0 eq) in DMF (10 mL) was added NaH (172.0 mg, 4.3 mmol, 3.62 eq) at 0° C. under N₂, stirred for 30 minutes, then was added a solution of I₂ (238.0 mg, 0.94 mmol, 0.79 eq) in DMF (2 mL) at 20° C. and stirred for 15.5 hrs at 25° C. The reaction mixture was cooled to r.t., EtOAc (30 mL) and water (30 mL) were added and layers were separated. The aqueous phase was extracted with EtOAc (30 mL×2). Combined extracts were washed with brine (40 mL), dried over Na₂SO₄, filtered, and concentrated under vacuum to give a residue. The residue was purified over column chromatography (petroleum ether/EtOAc=1:1) to give the title compound (70.0 mg, 20% yield) as yellow oil. LCMS: m/z 296.8 [M−H]−, ESI neg.

### Step B: Methyl 3-chloro-5-hydroxy-4-iodobenzoate

To a solution of 3-chloro-5-hydroxy-4-iodo-benzoic acid (3.5 g, 11.73 mmol, 1.0 eq) in methanol (35 mL) was added sulfuric acid (664.99 mg, 6.64 mmol, 0.57 eq). The mixture was stirred at 80° C. for 6 hours. The reaction mixture was cooled to r.t., EtOAc (60 mL) and water (60 mL) were added and layers were separated. The aqueous phase was extracted with EtOAc (70 mL×2). Combined extracts were washed with brine (80 mL), dried over Na₂SO₄, filtered, and concentrated under vacuum. The crude was purified by column (PE:EA=5:1 to 1:1) to give the title compound (3.30 g, 90% yield) was obtained as a yellow solid. LCMS: m/z 310.9 [M−H]⁻, ESI neg.

### Step C: 3-Chloro-5-hydroxy-4-iodobenzamide

To a solution of methyl 3-chloro-5-hydroxy-4-iodo-benzoate (3.3 g, 10.6 mmol, 1.0 eq) in NH₃/MeOH (40.0 mL, 280 mmol, 26.5 eq) was added TBD (444 mg, 3.19 mmol, 0.3 eq). The mixture was stirred at 50° C. for 12 hours in a sealed tube. The reaction mixture was cooled to r.t., EtOAc (60 mL) and water (60 mL) were added, and layers were separated. The aqueous phase was extracted with EtOAc (60 mL×2). Combined extracts were washed with brine (60 mL), dried over Na₂SO₄, filtered, and concentrated under vacuum to give a yellow solid. The solid was purified by column (PE:EA=5:1 to 1:1) to give the title compound (820.0 mg, 2.76 mmol, 26% yield) as yellow solid. LCMS: m/z 297.9 [M−H]⁻, ESI neg.

### Step D: 3-Chloro-5-hydroxy-4-iodobenzonitrile

To a solution of 3-chloro-5-hydroxy-4-iodo-benzamide (100.0 mg, 0.34 mmol, 1.0 eq) in THF (3 mL) was added SOCl₂ (0.12 mL, 1.68 mmol, 5.0 eq). The mixture was stirred at 50° C. for 12 hours. The reaction mixture was cooled to r.t., EtOAc (20 mL) and water (20 mL) were added, and layers were separated. The aqueous phase was extracted with EtOAc (20 mL×2). Combined extracts were washed with brine (20 mL), dried over Na₂SO₄, filtered, and concentrated under vacuum. The crude was purified by column chromatography (PE:EA=10:1 to 1:1) to give the title compound (70.0 mg, 75% yield) as yellow solid. LCMS: m/z 277.9 [M−H]−, ESI neg.

### Step E: (2-Chloro-4-cyano-6-hydroxyphenyl)boronic acid

To a mixture of 3-chloro-5-hydroxy-4-iodo-benzonitrile (70.0 mg, 0.25 mmol, 1.0 eq), 4,4,4′,4′,5,5,5′,5′-octamethyl-2,2′-bi(1,3,2-dioxaborolane) (253.5 mg, 1.0 mmol, 4.0 eq) in 1,4-dioxane (1 mL) was added KOAc (73.8 mg, 0.75 mmol, 3.0 eq) and Pd(dppf)Cl₂ (18.31 mg, 0.03 mmol, 0.1 eq), then the mixture was stirred at 95° C. for 4 hours. The reaction mixture was cooled to r.t, EtOAc (20 mL) and water (20 mL) were added, and layers were separated. The aqueous phase was extracted with EtOAc (20 mL×2). Combined extracts were washed with brine (20 mL), dried over Na₂SO₄, filtered, and concentrated under vacuum. The crude was purified by column (PE:EA=10:1 to 1:1) to give the title compound (40.0 mg, 57% yield) as white solid.

### Step F: 3-Chloro-4-[2-[[(3R)-1-ethyl-3-piperidyl]amino]oxazolo[4,5-b]pyridin-5-yl]-5-hydroxy-benzonitrile;2,2,2-trifluoroacetic acid

To a solution of (2-chloro-4-cyano-6-hydroxy-phenyl)boronic acid (40.0 mg, 0.2 mmol, 1.0 eq) and 5-bromo-N-[(3R)-1-ethyl-3-piperidyl]oxazolo[4,5-b]pyridin-2-amine;2,2,2-trifluoroacetic acid (Example 21, step H) (106.8 mg, 0.24 mmol, 1.2 eq) in 1,4-dioxane (1 mL) and water (0.2 mL) was added CsF (123.1 mg, 0.81 mmol, 4.0 eq) and XPhos Pd G3 (17.17 mg, 0.02 mmol, 0.1 eq) under N2. The mixture was stirred at 95° C. for 2 hours. The reaction mixture was cooled to r.t. The mixture was filtered, and concentrated under vacuum to give a residue which was purified by column chromatography (C₁₈, 0.1% TFA in water/MeCN) to afford the title compound (2.91 mg, 3% yield) as a yellow solid. LCMS: m/z 398.0 [M+H]⁺, ESI pos.

### Example 108

### 4-[2-[[(1R,2R)-2-(Dimethylamino)cyclopentyl]amino]oxazolo[4,5-b]pyridin-5-yl]-3-hydroxy-5-methyl-benzonitrile

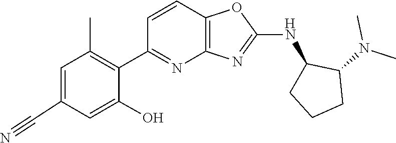

### Step A: N-[(1R,2R)-2-[(5-Chlorooxazolo[4,5-b]pyridin-2-yl)amino]cyclopentyl]carbamic acid tert-butyl ester

To a mixture of 5-chloro-2-(methylthio)oxazolo[4,5-b]pyridine (120 mg, 599 μmol, 1.0 eq) and N-[(1R,2R)-2-aminocyclopentyl]carbamic acid tert-butyl ester (155.7 mg, 777 μmol, 1.3 eq) in 1,4-dioxane (1.67 mL) was added triethylamine (108 μL, 777 μmol, 1.3 eq). The reaction mixture was stirred overnight at 120° C. After cooling to r.t., it was extracted with EtOAc and water. The aqueous layers were back-extracted with EtOAc. The combined organic layers were washed with water and brine, then dried over anhydrous sodium sulfate, filtered and concentrated in vacuo. The crude (280 mg) was used in the next step without further purification. LCMS m/z: 353.2 [M+H⁺, ESI pos.

### Step B: N-[(1R,2R)-2-[[5-(4-cyano-2-hydroxy-6-methyl-phenyl)oxazolo[4,5-b]pyridin-2-yl]amino]cyclopentyl]carbamic acid tert-butyl ester

To a mixture of N-[(1R,2R)-2-[(5-chlorooxazolo[4,5-b]pyridin-2-yl)amino]cyclopentyl]-carbamic acid tert-butyl ester (280 mg, 0.778 mmol, 1.0 eq) and (4-cyano-2-hydroxy-6-methyl-phenyl)boronic acid (Example 25, step D) (234 mg, 1.32 mmol, 1.7 eq) in 1,4-dioxane (4.8 mL) and water (1.2 mL) was added cesium carbonate (760 mg, 2.33 mmol, 3.0 eq), followed by the addition of XPhos Pd G3 (98.75 mg, 116.66 μmol, 0.15 eq) under argon atmosphere. The reaction mixture was stirred at 100° C. for 5 hours. The reaction mixture was extracted with ethyl acetate, ammonium chloride and water. The aqueous layers were back-extracted twice with ethyl acetate. The combined organic layers were washed with water and brine, then dried with anhydrous sodium sulfate, filtered and concentrated in vacuo. The crude was purified by flash chromatography (gradient 0-50% ethyl acetate in heptane) to afford the title compound (273.1 mg, 77% yield) as light brown viscous oil. LCMS m/z: 450.3 [M+H]⁺, ESI pos.

### Step C: 4-[2-[[(1R,2R)-2-aminocyclopentyl]amino]oxazolo[4,5-b]pyridin-5-yl]-3-hydroxy-5-methyl-benzonitrile;1:1 hydrogen chloride

To a solution of N-[(1R,2R)-2-[[5-(4-cyano-2-hydroxy-6-methyl-phenyl)oxazolo[4,5-b]pyridin-2-yl]amino]cyclopentyl]carbamic acid tert-butyl ester (273.1 mg, 607.6 μmol, 1.0 eq) in methanol (2.5 mL) and DCM (5 mL) was added dropwise 4 M HCl in dioxane (1.52 mL, 6.08 mmol, 10 eq). The reaction was stirred at r.t. overnight under an atmosphere of argon. The solvents were evaporated in vacuo to afford the title compound (233.9 mg, 96%) as off-white solid. LCMS m/z: 350.2 [M+H]⁺, ESI pos.

### Step D: 4-[2-[[(1R,2R)-2-(Dimethylamino)cyclopentyl]amino]oxazolo[4,5-b]pyridin-5-yl]-3-hydroxy-5-methyl-benzonitrile

To a suspension of N-[(1R,2R)-2-[[5-(4-cyano-2-hydroxy-6-methyl-phenyl)oxazolo[4,5-b]pyridin-2-yl]amino]cyclopentyl]carbamic acid tert-butyl ester (90 mg, 224 μmol, 1.0 eq) in 1,2-dichloroethane (2.4 mL) was added formaldehyde (408 mg, 36.7 μL, 492.6 μmol, 2.2 eq) and sodium acetate (45.9 mg, 559.8 μmol, 2.5 eq). The mixture was cooled with an ice bath and sodium triacetoxyborohydride (189.8 mg, 895.7 μmol, 4.0 eq) was added. After 5 mins, the reaction mixture (still a suspension) was allowed to come to r.t. stirring was continued for 2 h. Afterwards, the reaction mixture was extracted with sodium bicarbonate, water and DCM. The aqueous layers were back-extracted twice with DCM. The combined organic layers were dried over anhydrous sodium sulfate, filtered and concentrated in vacuo. The crude (77 mg, light yellow powder) was purified via SFC (column chiral AD-H, 5 μm, 250×20 mm, 25% MeOH+0.2% DEA) to afford the title compound (51 mg, 60% yield) as off-white solid. LCMS m/z: 378.3 [M+H]⁺, ESI pos.

### Example 109

### 4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid

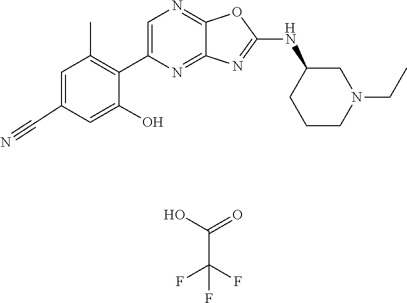

### Step A: 3-(Benzyloxy)-6-chloropyrazin-2-amine

To a stirred solution of benzyl alcohol (4.49 mL, 43.18 mmol, 1.0 eq) in THF (90 mL) at r.t. was added NaH (1.73 g, 43.18 mmol, 1.0 eq, 60%), The mixture was stirred at ambient temperature for 10 min. 3-Bromo-6-chloropyrazin-2-amine (9.0 g, 43.18 mmol, 1.0 eq; CAS: 212779-21-0) was then added and the reaction mixture was heated at 70° C. for 4 hours. The reaction mixture was cooled to r.t. Ethyl acetate (60 mL) and water (80 mL) were added and layers were separated. The aqueous phase was extracted with ethyl acetate (80 mL×2). Combined extracts were washed with brine (80 mL), dried over Na₂SO₄, filtered, and concentrated under reduced pressure. The residue was purified by column chromatography on silica gel (petroleum ether:ethyl acetate=5:1 to 1:1), then purified by normal phase flash (Column Welch Ultimate XB-CN 250*70*10 um, condition: Hexane-EtOH Begin B 12 End B 12; Gradient Time(min) 15 100% B Hold Time(min) 5 FlowRate (mL/min) 140) to afford the title compound (2.3 g, 23% yield) as a yellow solid. LCMS: m/z 236.0 [M+H]⁺, ESI pos.

### Step B: Benzyl (R)-3-isothiocyanatopiperidine-1-carboxylate

To a solution of benzyl (R)-3-aminopiperidine-1-carboxylate (1.0 g, 4.27 mmol, 1.0 eq) in DCM (5 mL) was added a solution of thiophosgene (0.490 g, 4.27 mmol, 1.0 eq) in DCM (5 mL) dropwise at 0° C., then stirred at 20° C. for 2 hrs. The above reaction mixture was purified by silica gel column directly (petroleum ether:ethyl acetate=1:0 to 5:1) to afford the title compound (0.85 g, 3.08 mmol, 72% yield) as a yellow oil. ¹H NMR (DMSO-d₆, 400 MHz) δ [ppm]: 7.45-7.27 (m, 5H), 5.12 (s, 2H), 4.06-3.98 (m, 1H), 3.96-3.62 (m, 2H), 3.44-3.36 (m, 1H), 3.28-2.95 (m, 1H), 1.95-1.82 (m, 2H), 1.69-1.45 (m, 2H).

### Step C: Benzyl (R)-3-(3-(3-(benzyloxy)-6-chloropyrazin-2-yl)thioureido)piperidine-1-carboxylate

To a solution of 3-(benzyloxy)-6-chloropyrazin-2-amine (0.600 g, 2.55 mmol, 1.0 eq) in THF (6 mL) was added NaH (203.67 mg, 5.09 mmol, 2.0 eq) at 0° C., stirred at 0° C. for 0.5 hour, then the solution of benzyl (R)-3-isothiocyanatopiperidine-1-carboxylate (0.703 g, 2.55 mmol, 1.0 eq) in THF (3 mL) was added dropwise at 0° C. and stirring was continued at 20° C. for 1 hour. The above reaction solution was quenched with saturated ammonium chloride (100 mL), extracted with ethyl acetate (20 mL×3). The combined organic phases were washed with brine (50 mL), dried over anhydrous sodium sulfate, filtered and the filtrate was concentrated under reduced pressure The residue was purified by column chromatography on silica gel (petroleum ether:ethyl acetate=1:0 to 5:1) to afford the title compound (0.500 g, 36% yield) as a yellow gum. LCMS: m/z 512.1 [M+H]⁺, ESI pos.

### Step D: (R)-1-(6-Chloro-3-hydroxypyrazin-2-yl)-3-(piperidin-3-yl)thiourea

To a solution of benzyl (R)-3-(3-(3-(benzyloxy)-6-chloropyrazin-2-yl)thioureido)piperidine-1-carboxylate (0.50 g, 0.98 mmol, 1.0 eq) in TFA (1.0 mL, 13.2 mmol, 13.47 eq) was added anisole (0.21 mL, 1.95 mmol, 2.0 eq), then stirred at 75° C. for 12 hrs. The above reaction solution was concentrated under reduced pressure and purified by reversed phase flash (0.1% TFA, water-ACN) to yield the title compound (0.060 mg, 12% yield) as a yellow solid. LCMS: m/z 288.2 [M+H]⁺, ESI pos.

### Step E: (R)-1-(6-Chloro-3-hydroxypyrazin-2-yl)-3-(1-ethylpiperidin-3-yl)thiourea

To a solution of (R)-1-(6-chloro-3-hydroxypyrazin-2-yl)-3-(piperidin-3-yl)thiourea (0.040 g, 0.1 mmol, 1.0 eq) in methanol (1 mL) was added TEA (0.020 g, 0.2 mmol, 2.0 eq), stirred for 30 min, then acetic acid (0.030 g, 0.5 mmol, 5.0 eq), 4A MS (0.050 g) and acetaldehyde (0.07 mL, 0.5 mmol, 5.0 eq) were added, stirred at 20° C. for 20 min. Afterwards, NaBH₃CN (0.032 g, 0.5 mmol, 5.0 eq) was added and stirring was continued at 20° C. for 1 hour. The reaction mixture was filtered. The filtrate was concentrated. The crude was purified by reversed phase flash (0.1% TFA, water-ACN) to afford the title compound (0.040 g, 92% yield) as a yellow solid. LCMS: m/z 315.9 [M+H]⁺, ESI pos.

### Step F: (R)-5-Chloro-N-(1-ethylpiperidin-3-yl)oxazolo[4,5-b]pyrazin-2-amine

To a mixture of (R)-1-(6-chloro-3-hydroxypyrazin-2-yl)-3-(1-ethylpiperidin-3-yl)thiourea (0.030 g, 0.02 mmol, 1.0 eq) and tetrabutylazanium;iodide (0.008 g, 0.02 mmol, 1.0 eq) in THF (0.5 mL) was added H₂O₂ (0.005 g, 0.05 mmol, 2.0 eq) and stirred at 25° C. for 2 hours. Then the above reaction solution was purified by reversed phase flash (0.1% TFA, water-ACN) to afford the title compound (0.020 g, 71% yield) as yellow oil. LCMS: m/z 282.0 [M+H]⁺, ESI pos.

### Step G: 4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid

To a solution of (R)-5-chloro-N-(1-ethylpiperidin-3-yl)oxazolo[4,5-b]pyrazin-2-amine (Example 109, step F) (0.010 g, 0.03 mmol, 1.0 eq) in 1,4-dioxane (1 mL) and water (0.2 mL) was added (4-cyano-2-hydroxy-6-methylphenyl)boronic acid (0.004 g, 0.03 mmol, 1.0 eq), CsF (0.009 g, 0.06 mmol, 2.5 eq) and Xphos Pd G3 (0.004 g, 0.01 mmol, 0.2 eq) and the above reaction mixture was stirred at 95° C. for 2 hours under N2 atmosphere. After cooling to r.t., the reaction mixture was filtered and the filtrate was concentrated under reduced pressure. The crude was purified by reversed phase flash (0.1% TFA, water-MeCN) and re-purified by prep-HPLC (Column Waters Xbridge C₁₈ 150*50 mm*10 μm Condition water (NH₄HCO₃)-ACN Begin B 26 End B 56 Gradient Time(min) 10 100% B Hold Time(min)2 FlowRate (mL/min)30) to afford the title compound (0.001 g, 7.4% yield) as a white solid. LCMS: m/z 379.1 [M+H]⁺, ESI pos.

### Example 110

### 4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid

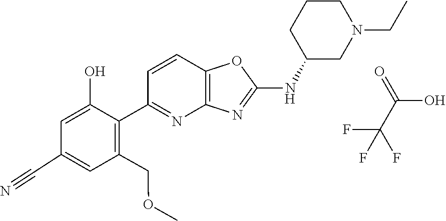

### Step A: Methyl 2-amino-5-bromo-3-methoxybenzoate

To a solution of methyl 2-amino-3-methoxy-benzoate (10.0 g, 55.2 mmol, 1.0 eq CAS: 5121-34-6) in methanol (40 mL) was added the solution of Br₂ (3.4 mL, 66.2 mmol, 1.2 eq) in acetic acid (20 mL) dropwise at 0-10° C. The reaction mixture was stirred at 20° C. for 2 hours. The reaction mixture was poured into aq. NaHSO₃ (20 mL) and extracted with EtOAc (150 mL×3). The organic phase was washed with brine (20 mL), dried over Na₂SO₄, filtered and concentrated under reduced pressure. The residue was purified by column chromatography (silica gel, petroleum ether/ethyl acetate=100/1 to 5/1) to give the title compound (10.0 g, 70% yield) was as a yellow solid. ¹H NMR (400 MHz, DMSO-d₆) δ [ppm]: 7.43 (d, 1H), 7.10 (d, 1H), 6.46 (br. s, 1H), 3.85 (s, 3H), 3.80 (s, 3H).

### Step B: Methyl 2-amino-5-cyano-3-methoxybenzoate

To a solution of methyl 2-amino-5-bromo-3-methoxy-benzoate (10.0 g, 38.45 mmol, 1.0 eq) in NMP (50 mL) was added CuCN (4.13 g, 46.14 mmol, 1.2 eq), The mixture was stirred at 180° C. for 2 hours under MW. The reaction mixture was poured into water (10 mL) and extracted with ethyl acetate (100 ml×3). The organic phase was washed with brine (100 mL×3), dried over Na₂SO₄, filtered and concentrated under reduced pressure. The residue was purified by column chromatography on silica gel (petroleum ether:ethyl acetate=1:0 to 5:1) to afford the title compound (6.0 g, 76% yield) as a white solid. LCMS: m/z 207.1 [M+H]⁺, ESI pos.

### Step C: Methyl 2-bromo-5-cyano-3-methoxybenzoate

To a solution of methyl 2-amino-5-cyano-3-methoxy-benzoate (5.7 g, 27.64 mmol, 1.0 eq), CuBr (5.95 g, 41.5 mmol, 1.5 eq) in MeCN (80 mL) was added butyl nitrite (4.28 g, 41.5 mmol, 1.5 eq), The mixture was stirred at 65° C. for 2 hours. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography on silica gel (petroleum ether:ethyl acetate=1:0 to 5:1) to afford the title compound (4.0 g, 54% yield) as a white solid. ¹H NMR (400 MHz, DMSO-d₆) δ [ppm]: 7.78 (d, 1H), 7.74 (d, 1H), 3.96 (s, 3H), 3.88 (s, 3H).

### Step D: 2-Bromo-5-cyano-3-hydroxybenzoic acid

To a solution of methyl 2-bromo-5-cyano-3-methoxy-benzoate (3.8 g, 14.1 mmol, 1.0 eq) in DCM (20 mL) was added BBr₃ (13.6 mL, 140.7 mmol, 10.0 eq) at −65° C., then the mixture was stirred at 25° C. for 16 hrs. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography on silica gel (petroleum ether:ethyl acetate=1:0 to 2:1) to afford the title compound (2.9 g, 85% yield) as yellow solid. ¹H NMR (400 MHz, DMSO-d₆) δ [ppm]: 13.76 (br. s, 1H), 11.41 (s, 1H), 7.52 (d, 1H), 7.33 (d, 1H).

### Step E: 2-Trimethylsilylethoxymethyl 2-bromo-5-cyano-3-(2-trimethylsilylethoxymethoxy)-benzoate

A solution of 2-bromo-5-cyano-3-hydroxy-benzoic acid (2.9 g, 11.9 mmol, 1.0 eq), Cs₂CO₃ (7.81 g, 23.96 mmol, 2.0 eq) in DMF (50 mL) was stirred for 10 mins at 25° C., then the SEM-Cl (4.0 g, 23.96 mmol, 2.0 eq) was added and stirred at 25° C. for 2 hours. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography (silica gel, petroleum ether:ethyl acetate=1:0 to 2:1) to afford the title compound (3.60 g, 60% yield) as a white solid. ¹H NMR (400 MHz, DMSO-d₆) δ [ppm]: 7.83 (d, 1H), 7.78 (d, 1H), 5.49 (d, 4H), 3.82-3.73 (m, 4H), 0.96-0.87 (m, 4H), −0.01 (s, 9H), −0.07 (s, 9H).

### Step F: 4-Bromo-3-(hydroxymethyl)-5-((2-(trimethylsilyl)ethoxy)methoxy)benzonitrile

To a solution of 2-trimethylsilylethoxymethyl 2-bromo-5-cyano-3-(2-trimethylsilylethoxymethoxy)benzoate (3.0 g, 5.97 mmol, 1.0 eq) in THF (20 mL) was added NaBH₄ (903.31 mg, 23.9 mmol, 4.0 eq) and stirred for 16 hrs at 25° C. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography (silica gel, petroleum ether:ethyl acetate=1:0 to 5:1) to afford the title compound (1.6 g, 75% yield) as a white solid. ¹H NMR (CD₃OD, 400 MHz) δ [ppm]: 7.55 (d, 1H), 7.48 (d, 1H), 5.65 (t, 1H), 5.43 (s, 2H), 4.52 (d, 2H), 3.73 (t, 2H), 0.88 (t, 2H), −0.05 (s, 9H).

### Step G: 4-Bromo-3-(methoxymethyl)-5-((2-(trimethylsilyl)ethoxy)methoxy)benzonitrile

To a solution of 4-bromo-3-(hydroxymethyl)-5-(2-trimethylsilylethoxymethoxy)benzonitrile (200 mg, 0.56 mmol, 1.0 eq) in THF (3 mL) was added NaH (44.7 mg, 1.12 mmol, 2.0 eq 60%) and stirred for 10 mins at 25° C., then Mel (0.1 mL, 1.61 mmol, 2.88 eq) was added and stirred for 2 hours at 25° C. The mixture was quenched with water (30 mL), and extracted with EtOAc (30 mL×3). The organic phase was washed with brine (30 mL×2), dried over anhydrous Na₂SO₄, filtered and concentrated in vacuum. The residue was purified by column chromatography on silica gel (PE/EtOAc, 50:1 to 10:1) to give the title compound (130 mg, 63% yield) was obtained as colorless oil. ¹H NMR (DMSO-d₆, 400 MHz) δ [ppm]: 7.60 (s, 1H), 7.46 (s, 1H), 5.44 (s, 2H), 4.48 (s, 2H), 3.74 (t, 2H), 3.39 (s, 1H), 0.88 (t, 2H), −0.04 (s, 9H).

### Step H: 3-(Methoxymethyl)-4-(4,4,5,5-tetramethyl-1,3,2-dioxaborolan-2-yl)-5-((2-(trimethylsilyl)ethoxy)methoxy)benzonitrile

To a mixture of 4-bromo-3-(methoxymethyl)-5-(2-trimethylsilylethoxymethoxy)benzonitrile (130.0 mg, 0.35 mmol, 1.0 eq), 4,4,4′,4′,5,5,5′,5′-octamethyl-2,2′-bi(1,3,2-dioxaborolane) (441.68 mg, 1.75 mmol, 5.0 eq) in 1,4-dioxane (4 mL) was added KOAc (102.8 mg, 1.05 mmol, 3.0 eq) and Pd(dppf)Cl₂ (25.5 mg, 0.03 mmol, 0.1 eq), then the mixture was stirred at 95° C. for 4 hours. The reaction mixture was poured into water (10 mL) and extracted with ethyl acetate (100 mL×3). The organic phase was washed with brine (100 mL×3), dried over Na₂SO₄, filtered and concentrated under reduced pressure. The residue was purified by column chromatography on silica gel (petroleum ether:ethyl acetate=1:0 to 10:1) to afford the title compound (20.0 mg, 14% yield) as a white solid. ¹H NMR (CD₃OD, 400 MHz) δ [ppm]: 7.31 (s, 1H), 7.26 (s, 1H), 5.30 (s, 2H), 4.49 (s, 2H), 3.84-3.76 (m, 2H), 3.32 (s, 3H), 1.40 (s, 12H), 0.99-0.92 (m, 2H), 0.01 (s, 9H).

### Step I: (R)-4-(2-((1-Ethylpiperidin-3-yl)amino)oxazolo[4,5-b]pyridin-5-yl)-3 (methoxymethyl)-5-((2-(trimethylsilyl)ethoxy)methoxy)benzonitrile

To a mixture of (3S,5R)-5-[(5-bromooxazolo[4,5-b]pyridin-2-yl)amino]-1-ethyl-piperidin-3-ol;2,2,2-trifluoroacetic acid (28.22 mg, 0.06 mmol, 1.3 eq), 3-(methoxymethyl)-4-(4,4,5,5-tetramethyl-1,3,2-dioxaborolan-2-yl)-5-(2-trimethylsilylethoxymethoxy)benzonitrile (20.0 mg, 0.05 mmol, 1.0 eq), K₂CO₃ (16.5 mg, 0.12 mmol, 2.5 eq) in 1,4-dioxane (1 mL) and water (0.2 mL) was added XPhos Pd G3 (4.01 mg, 0.1 eq) and purged with N2 three times. The mixture was heated at 95° C. for 2 hrs. The mixture was then cooled to 25° C., concentrated under reduced pressure and the crude was purified by column chromatography (Cis, 0.1% TFA in water/MeCN) to give the title compound (15.0 mg, 85% yield) as a yellow solid. LCMS: m/z 538.4 [M+H]⁺, ESI pos.

### Step J: 4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid

To a mixture of 4-[2-[[(3R)-1-ethyl-3-piperidyl]amino]oxazolo[4,5-b]pyridin-5-yl]-3-(methoxymethyl)-5-(2-trimethylsilylethoxymethoxy)benzonitrile (15.0 mg, 0.03 mmol, 1.0 eq) in DCM (1 mL) was added TFA (27.06 mg, 0.28 mmol, 10.0 eq) and stirred for 2 hours at 25° C. The mixture was concentrated under reduced pressure and purified by column chromatography (C₁₈, 0.1% TFA in water/MeCN) to afford the title compound (7.7 mg, 52% yield) as a yellow solid. LCMS: m/z 408.1 [M+H]+, ESI pos.

### Example 111

### 4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid

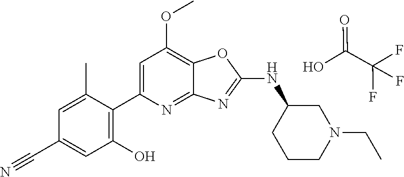

### Step A: R-5-Chloro-N-(1-ethylpiperidin-3-yl)-7-iodooxazolo[4,5-b]pyridin-2-amine: trifluoroacetic acid

To a mixture of R-1-methyl piperidine-3-amine (81.4 mg, 0.64 mmol, 2.5 eq) in MeCN (3 mL) was added 2,5-dichloro-7-iodooxazolo[4,5-b]pyridine (80.0 mg, 0.25 mmol, 1.0 eq) and DIEA (0.08 mL, 0.51 mmol, 2.0 eq) and the mixture was stirred at 25° C. for 1 h. The reaction mixture was diluted with 2 mL MeOH. Afterwards, the residue was purified by column chromatography (C₁₈, 0.1% TFA in water/MeCN) and re-purified by prep-HPLC (column: 3_Phenomenex Luna C18 75*30 mm*3 um; Condition: water(TFA)-CAN; Begin B: 15; End B: 35; Gradient Time(min): 9; 100% B Hold Time(min): 2; FlowRate (ml/min): 25) to afford the title compound (70.0 mg, 68% yield, TFA salt) as a yellow solid. LCMS: m/z 407.1 [M+H]⁺ ESI pos.

### Step B: R-5-Chloro-N-(1-ethylpiperidin-3-yl)-7-methoxyoxazolo[4,5-b]pyridin-2-amine

A mixture of 1,10-phenanthroline (3.46 mg, 0.02 mmol, 0.2 eq), R-5-chloro-N-(1-ethylpiperidin-3-yl)-7-iodooxazolo[4,5-b]pyridin-2-amine;trifluoroacetic acid (50.0 mg, 0.1 mmol, 1.0 eq), Cs₂CO₃ (62.42 mg, 0.19 mmol, 2.0 eq) and CuI (1.82 mg, 0.01 mmol, 0.1 eq) in Methanol (0.5 mL). N₂ was bubbled into the reaction mixture for 3 minutes. Then the reaction vessel was sealed and heated in a microwave at 110° C. for 60 mins. The reaction mixture was cooled to r.t. and concentrated under reduced pressure. The residue was purified by column chromatography (C₁₈, 0.1% TFA in water/MeCN) and re-purified by column chromatography (C₁₈, 0.1% NH₃·H₂O in water/MeCN) to afford the title compound (10.0 mg, 26% yield) as yellow oil. LCMS: m/z 311.1 [M+H]+, ESI pos.

### Step C: 4-[2-[[(3R)-1-Ethyl-3-piperidyl]amino]-7-methoxy-oxazolo[4,5-b]pyridin-5-yl]-3-hydroxy-5-methyl-benzonitrile;2,2,2-trifluoroacetic acid

To a solution of (4-cyano-2-hydroxy-6-methyl phenyl)boronic acid (13.67 mg, 0.08 mmol, 3.0 eq) and R-5-chloro-N-(1-ethylpiperidin-3-yl)-7-methoxyoxazolo[4,5-b]pyridin-2-amine (8.0 mg, 0.03 mmol, 1.0 eq) in 1,4-dioxane (1 mL) and water (0.2 mL) was added CsF (20.0 mg, 0.13 mmol, 5.11 eq) and XPhos Pd G3 (2.18 mg, 0.0 mmol, 0.1 eq) under N₂. The mixture was stirred at 95° C. for 2 hours. Afterwards, the reaction mixture was cooled to r.t. EtOAc (10 mL) and water (10 mL) were added and layers were separated. The aqueous phase was extracted with EtOAc (10 mL×2). Combined extracts were washed with brine (20 mL), dried over Na₂SO₄, filtered, and concentrated under reduced pressure. The residue was purified by prep-HPLC (Method: column: Waters Xbridge 150*25 mm*5 um; Condition: water (NH₄HCO₃)-ACN Begin B: 36; End B: 66; Gradient Time (min): 9; 100% B Hold Time(min): 2; FlowRate (ml/min): 25.). After lyophilization, the compound was re-purified by prep-HPLC (Method: Column: YMC Triart C18 150*25 mm*5 um; Condition: water(TFA)-ACN, Begin B: 19; End B: 39; Gradient Time(min): 10; 100% B Hold Time(min): 2; FlowRate (ml/min): 25.) to afford the title compound (1.06 mg, 10% yield) as a yellow solid. LCMS: m/z 408.1 [M+H]⁺, ESI pos.

### Example 112

### 5-Chloro-2-[2-[[(1R,2R)-2-(dimethylamino)cyclopentyl]amino]oxazolo[4,5-b]pyridin-5-yl]-3-methyl-phenol

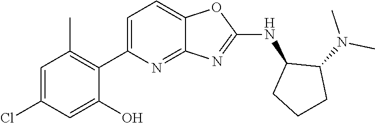 [SMILES: Cc1cc(Cl)cc(O)c1-c1ccc2oc(N[C@@H]3CCC[C@H]3N(C)C)nc2n1]

### Step A: N-[(1R,2R)-2-[(5-chlorooxazolo[4,5-b]pyridin-2-yl)amino]cyclopentyl]carbamic acid tert-butyl ester

Following to GP1, 5-chloro-2-(methylthio)oxazolo[4,5-b]pyridine (115 mg, 573.1 μmol, 1.0 eq) and N-[(1R,2R)-2-aminocyclopentyl]carbamic acid tert-butyl ester (126.27 mg, 630.45 μmol, 1.1 eq) were reacted to yield the title compound (240 mg, 107%) as light brown crystalline, that was used in the next step without further purification.

### Step B: N-[(1R,2R)-2-[[5-(4-chloro-2-hydroxy-6-methyl-phenyl)oxazolo[4,5-b]pyridin-2-yl]amino]cyclopentyl]carbamic acid tert-butyl ester

Following to GP2a, N-[(1R,2R)-2-[(5-chlorooxazolo[4,5-b]pyridin-2-yl)amino]cyclopentyl]-carbamic acid tert-butyl ester (240 mg, 612.2 μmol, 1.0 eq) and (4-chloro-2-hydroxy-6-methyl-phenyl)boronic acid (CAS #1207961-50-9) were reacted to afford the title compound (195.1 mg, 62%) as light yellow solid. LCMS: 459.3 [M+H]⁺, ESI pos.

### Step C: 2-[2-[[(1R,2R)-2-Aminocyclopentyl]amino] oxazolo[4,5-b]pyridin-5-yl]-5-chloro-3-methyl-phenol

To a solution of N-[(1R,2R)-2-[[5-(4-chloro-2-hydroxy-6-methyl-phenyl)oxazolo[4,5-b]pyridin-2-yl]amino]cyclopentyl]carbamic acid tert-butyl ester (190.5 mg, 373.58 umol, 1.0 eq) in DCM (3.2 mL) and methanol (1.6 mL) was added dropwise 4 M HCl in dioxane (1.11 g, 0.9 mL, 3.74 mmol, 10 eq). The reaction mixture was stirred at r.t. under an atmosphere of argon for 4.5 hours. The solvent was removed under reduced pressure. Afterwards, the residue was dissolved in water and extracted with DCM. The organic phase was then washed with sodium bicarbonate and water. The aqueous layers were re-extracted with DCM. The combinated organic layers were washed with water and brine, then dried with sodium sulfate, filtered and concentrated in vacuo. The crude was purified by flash chromatography (gradient 0-80% DCM:MeOH:NH₄OH (110:10:1) in DCM) to afford the title compound (93.7 mg, 69%) as white solid. LCMS: 359.1 [M+H]⁺, ESI pos.

### Step D: 5-Chloro-2-[2-[[(1R,2R)-2-(dimethylamino)cyclopentyl]amino]oxazolo[4,5-b]pyridin-5-yl]-3-methyl-phenol

According to GP4, aforementioned 2-[2-[[(1R,2R)-2-aminocyclopentyl]amino]oxazolo[4,5-b]pyridin-5-yl]-5-chloro-3-methyl-phenol was reacted to obtain the title compound after HPLC chromatography (Column: Gemini NX, 12 nm, 5 μm, 100×30 mm; MeCN/(water+0.1% TEA)) to afford the title compound as a white powder (34.3 mg, 40%). LCMS: 385.3 [M−H]⁻, ESI neg.

### Example 113

### 5-Chloro-2-[2-[[(3R)-1-ethyl-3-piperidyl]amino]oxazolo[4,5-b]pyrazin-5-yl]-3-methyl-phenol;2,2,2-trifluoroacetic acid

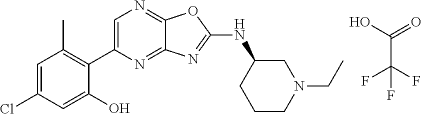

To a solution of aforementioned (R)-5-chloro-N-(1-ethylpiperidin-3-yl)oxazolo[4,5-b]pyrazin-2-amine (Example 109, step F) (0.010 g, 0.03 mmol, 1.0 eq) in 1,4-dioxane (1 mL) and water (0.2 mL) was added (4-chloro-2-hydroxy-6-methylphenyl)boronic acid (0.005 g, 0.03 mmol, 1.0 eq), CsF (0.009 g, 0.06 mmol, 2.5 eq) and XPhos Pd G3 (0.004 g, 0.01 mmol, 0.2 eq). Then the above reaction mixture was stirred at 90° C. for 2 hours under nitrogen atmosphere. The above reaction mixture was cooled to r.t., filtered and the filtrate was concentrated. The crude was subsequently purified by reversed phase flash (0.1% TFA, water-MeCN) to afford the title compound (0.002 g, 19% yield) as a yellow solid. LCMS: m/z 388.1 [M+H]+, ESI pos.

### Example 114

### 5-Chloro-2-[2-[[(3R)-1-ethyl-3-piperidyl]amino]oxazolo[4,5-b]pyridin-5-yl]-3-(trifluoromethoxy)phenol;2,2,2-trifluoroacetic acid

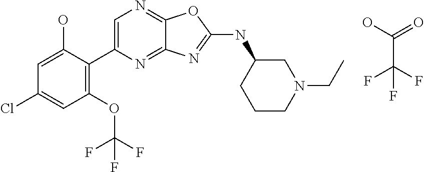

### Step A: 4-Chloro-2-iodo-6-(trifluoromethoxy) aniline

To a solution of 4-chloro-2-(trifluoromethoxy) aniline (CAS #175205-77-3, 9.10 g, 43.0 mmol, 1.0 eq) in acetic acid (50 mL) was added N-iodosuccinimide (9.6 g, 42.7 mmol, 0.99 eq) at 0° C., then stirred at 20° C. for 3 hours. The above reaction solution was diluted with water (200 mL), extracted with ethyl acetate (50 mL×3). The combined organic phase was washed with saturated NaHCO₃ solution (100 mL×2), dried over anhydrous sodium sulfate, filtered and the filtrate was concentrated under reduced pressure. The residue was purified by silica gel column chromatography (petroleum ether:ethyl acetate=1:0 to 100:1) to afford the title compound (10.0 g, 69% yield) as a yellow oil. ¹H NMR (CDCl₃, 400 MHz) δ [ppm]: 7.59 (d, 1H), 7.16 (d, 1H), 4.36 (br s, 2H).

### Step B: 2-(Benzyloxy)-4-chloro-6-(trifluoromethoxy) aniline

To a solution of benzyl alcohol (5.77 g, 53.3 mmol, 3.0 eq) in toluene (120 mL) was added Cs₂CO₃ (17.4 g, 53.3 mmol, 3.0 eq), then stirred at 20° C. for 30 mins. Then 4-chloro-2-iodo-6-(trifluoromethoxy) aniline (6.0 g, 17.8 mmol, 1.0 eq), 1,10-Phenanthroline (0.64 g, 3.56 mmol, 0.2 eq), CuI (0.34 g, 1.78 mmol, 0.1 eq), was added and stirred at 110° C. for 12 hours. The above reaction solution was diluted with water (200 mL), extracted with ethyl acetate (100 mL×3). The combined organic phase was washed with brine (200 mL), dried over anhydrous sodium sulfate, filtered and the filtrate was concentrated under reduced pressure. The residue was purified by silica gel column chromatography (petroleum ether:ethyl acetate=1:0 to 50:1) to get a black oil which was then purified by reversed phase flash (0.1% TFA, water-MeCN) to afford the title compound (2.2 g, 39% yield) as a yellow solid. LCMS: m/z 318.0 [M+H]+, ESI pos.

### Step C: 1-(Benzyloxy)-5-chloro-2-iodo-3-(trifluoromethoxy)benzene

To a mixture of 2-(benzyloxy)-4-chloro-6-(trifluoromethoxy) aniline (0.70 g, 2.2 mmol, 1.0 eq) in HCl (7.0 mL, 37% purity) was added the solution of NaNO₂ (0.30 g, 4.41 mmol, 2.0 eq) in water (3 mL) at 0° C., stirred at 0° C. for 0.5 h, then the solution of KI (1.46 g, 8.81 mmol, 4.0 eq) in water (4 mL) was added dropwise at 0° C. and stirring was continued at 20° C. for 12 hours. The mixture was quenched with saturated sodium sulfite (50 mL), extracted with ethyl acetate (20 mL×3). The combined organic layers were washed with brine (50 mL), dried over anhydrous sodium sulfate, filtered and the filtrate was concentrated under reduced pressure. The residue was purified by column chromatography (silica gel, petroleum ether:ethyl acetate=1:0 to 20:1) to afford the title compound (0.30 g, 32% yield) as a yellow solid. ¹H NMR (CDCl₃, 400 MHz) δ [ppm]: 7.53-7.48 (m, 2H), 7.47-7.40 (m, 2H), 7.39-7.33 (m, 1H), 7.00-6.95 (m, 1H), 6.82 (d, 1H), 5.18 (s, 2H).

### Step D: 2-(2-(Benzyloxy)-4-chloro-6-(trifluoromethoxy)phenyl)-4,4,5,5-tetramethyl-1,3,2-dioxaborolane

To a solution of 1-(benzyloxy)-5-chloro-2-iodo-3-(trifluoromethoxy)benzene (0.20 g, 0.47 mmol, 1.0 eq) in THF (1 mL) was added 2-isopropoxy-4,4,5,5-tetramethyl-1,3,2-dioxaborolane (0.22 mg, 1.18 mmol, 2.53 eq), then iprMgCl-LiCl (0.54 mL, 0.7 mmol, 1.5 eq) was dropped under −5° C., then stirred at 20° C. for 1 hour. The above reaction solution was quenched with saturated NH₄Cl solution (50 mL) at 0° C. and extracted with ethyl acetate (20 mL×3). The combined organic phase was washed with brine (50 mL), dried over anhydrous sodium sulfate, filtered and the filtrate was concentrated under reduced pressure. The residue was purified by silica gel column chromatography (petroleum ether:ethyl acetate=1:0 to 50:1) to afford the title compound (30.0 mg, 15% yield) as a yellow solid. ¹H NMR (CDCl₃, 400 MHz) δ [ppm]: 7.47-7.43 (m, 2H), 7.41-7.30 (m, 3H), 6.89 (s, 1H), 6.85 (s, 1H), 5.05 (s, 2H), 1.30 (s, 12H).

### Step E: (R)-5-(2-(benzyloxy)-4-chloro-6-(trifluoromethoxy)phenyl)-N-(1-ethylpiperidin-3-yl)oxazolo[4,5-b]pyridin-2-amine

To a solution of 2-(2-(benzyloxy)-4-chloro-6-(trifluoromethoxy)phenyl)-4,4,5,5-tetramethyl-1,3,2-dioxaborolane (0.030 g, 0.07 mmol, 1.0 eq) in 1,4-dioxane (1 mL) and water (0.2 mL) was added (R)-5-bromo-N-(1-ethylpiperidin-3-yl)oxazolo[4,5-b]pyridin-2-amine (0.031 g, 0.07 mmol, 1.0 eq), CsF (0.026 g, 0.17 mmol, 2.5 eq) and XPhos Pd G3 (0.012 g, 0.01 mmol, 0.2 eq), then the above reaction mixture was stirred at 90° C. for 1 h under N₂ atmosphere. After cooling down to ambient temperature, it was filtered and the filtrate was concentrated under reduced pressure. The residue was purified by prep-TLC (DCM:MeOH=10:1, Rf=0.1) to afford the title compound (0.020 mg, 29% yield) as a yellow gum. LCMS: m/z 547.3 [M+H]+, ESI pos.

### Step F: 5-Chloro-2-[2-[[(3R)-1-ethyl-3-piperidyl]amino]oxazolo[4,5-b]pyridin-5-yl]-3-(trifluoromethoxy)phenol;2,2,2-trifluoroacetic acid

The solution of (R)-5-(2-(benzyloxy)-4-chloro-6-(trifluoromethoxy)phenyl)-N-(1-ethylpiperidin-3-yl)oxazolo[4,5-b]pyridin-2-amine (0.010 mg, 0.02 mmol, 1.0 eq) in TFA (0.5 mL, 6.73 mmol, 368 eq) was stirred at 75° C. for 12 hrs. After cooling down to ambient temperature, concentrated under reduced pressure. The residue was purified by prep-HPLC (Column 3_Phenomenex Luna C₁₈ 75*30 mm*3 um Condition water (TFA)-MeCN Begin B 29End B 49 Gradient Time(min) 10100% B Hold Time(min) 2 FlowRate (ml/min) 25) to get a yellow solid. Then the above yellow solid was re-purified by prep-HPLC (Column Waters Xbridge C₁₈ 150*50 mm*10 um Condition water (NH4HCO3)-MeCN Begin B 50End B 80 Gradient Time(min) 10100% B Hold Time(min) 2 FlowRate (mL/min) 30) to afford the title compound (1.33 mg, 12% yield) as a white solid. LCMS: m/z 457.1 [M+H]⁺, ESI pos.

### Example 115

### 3-Hydroxy-5-methyl-4-[2-[[(2S)-2-hydroxy-1,2,3,5,6,7,8,8a-octahydroindolizin-8-yl]amino]oxazolo[4,5-b]pyridin-5-yl]benzonitrile;2,2,2-trifluoroacetic acid

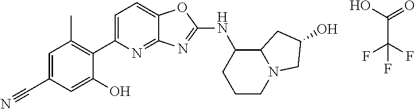 [SMILES: Cc1cc(C#N)cc(O)c1-c1ccc2oc(NC3CCCN4C[C@@H](O)CC34)nc2n1 ; O=C(O)C(F)(F)F]

### Step A: tert-Butyl (2-bromopyridin-3-yl)carbamate

To a solution of 2-bromopyridin-3-amine (10.0 g, 57.8 mmol, 1.0 eq) in THF (200 mL) LiHMDS (115.6 mL, 115.6 mmol, 2.0 eq) was added dropwise at 0° C., then stirred at 0° C. for 0.5 hour, then the solution of di-t-butyldicarbonate (13.3 g, 60.7 mmol, 1.05 eq) in THF (10 mL) was added slowly at 0° C. The reaction mixture was stirred at 20° C. for 12 hours. The above reaction solution was quenched with saturated ammonium chloride (500 mL), extracted with ethyl acetate (100 mL×3). The combined organic phases were washed with brine (200 mL), dried over anhydrous sodium sulfate, filtered and the filtrate was concentrated under reduced pressure. The residue was purified by silica gel column chromatography (petroleum ether:ethyl acetate=1:0 to 2:1) to afford the title compound (12.0 g, 70% yield) as a yellow oil. LCMS: m/z 275.0 [M+H]⁺, ESI pos.

### Step B: tert-Butyl (2-allylpyridin-3-yl)carbamate

To a solution of tert-butyl (2-bromopyridin-3-yl)carbamate (5.0 g, 18.31 mmol, 1.0 eq) in MeCN (400 mL) was added 2-allyl-4,4,5,5-tetramethyl-1,3,2-dioxaborolane (6.15 g, 36.61 mmol, 2.0 eq), Pd(dppf)Cl₂ (0.67 g, 0.92 mmol, 0.05 eq) and CsF (6.95 g, 45.77 mmol, 2.5 eq), then stirred at 70° C. for 2 hours. The above reaction solution was diluted with water (300 mL), extracted with ethyl acetate (100 mL×3). The combined organic phases were washed with brine (300 mL), dried over anhydrous sodium sulfate, filtered and the filtrate was concentrated under reduced pressure. The residue was purified by silica gel column chromatography (petroleum ether:ethyl acetate=1:0 to 5:1) to afford the title compound (3.80 g, 86% yield) as a yellow oil. LCMS: m/z 235.0 [M+H]⁺, ESI pos.

### Step C: tert-Butyl (S)-(2-(2,3-dihydroxypropyl)pyridin-3-yl)carbamate

To a solution of tert-butyl (2-allylpyridin-3-yl)carbamate (1.0 g, 4.27 mmol, 1.0 eq) in tert-butanol (40 mL) and water (40 mL) was added AD-mix-((8.31 g, 10.67 mmol, 2.5 eq) and methanesulfonamide (406.0 mg, 4.27 mmol, 1.0 eq). Stirring was continued at 20° C. for 4 hours, then another batch of AD-mix-a (8.31 g, 10.67 mmol, 2.5 eq) was added and the reaction was stirred at 20° C. for 12 hours. The above reaction solution was quenched with sodium sulfite (150 mL), extracted with ethyl acetate (50 mL×3). The combined organic phases were washed with brine (100 mL), dried over anhydrous sodium sulfate, filtered and the filtrate was concentrated under reduced pressure to give a yellow oil. The residue was purified by silica gel column chromatography (petroleum ether:ethyl acetate=1:0 to 10:1) to afford the title compound (600.0 mg, 52% yield) as a yellow solid. LCMS: m/z 269.1 [M+H]⁺, ESI pos.

### Step D: (S)-8-((tert-Butoxycarbonyl)amino)-2-hydroxy-2,3-dihydro-1H-indolizin-4-ium chloride

To a solution of tert-butyl (S)-(2-(2,3-dihydroxypropyl)pyridin-3-yl)carbamate (600.0 mg, 2.24 mmol, 1.0 eq) in DCM (9 mL) was added TEA (250.0 mg, 2.47 mmol, 1.1 eq), then the solution of ethanesulfonyl chloride (0.2 mL, 2.1 mmol, 0.94 eq) in DCM (3 mL) was added at 0° C., then stirred at 20° C. for 2 hours. The solvent of the reaction solution was removed by nitrogen flow to get a white solid, then purified by reversed phase flash (neutral, water-MeCN). Afterwards, the solvent was removed by lyophilization to afford the title compound (300 mg, 47% yield) as a yellow gum. LCMS: m/z 251.0 [M+H]⁺, ESI pos.

### Step E: tert-Butyl ((2S)-2-hydroxyoctahydroindolizin-8-yl)carbamate hydrochloride

To a solution of (S)-8-((tert-butoxycarbonyl)amino)-2-hydroxy-2,3-dihydro-1H-indolizin-4-ium chloride (300 mg, 1.05 mmol, 1.0 eq) in methanol (2 mL) was added PtO₂ (47.5 mg, 0.21 mmol, 0.2 eq), then stirred at 20° C. for 2 hours under H₂ atmosphere. The above reaction mixture was filtered and the filtrate was concentrated under reduced pressure to afford the title compound (250 mg, 0.85 mmol, crude product) as a yellow oil. ¹H NMR (CD₃OD, 400 MHz) δ [ppm]: 4.71-4.42 (m, 2H), 4.19-3.93 (m, 1H), 3.54-3.40 (m, 1H), 3.35-3.34 (m, 3H), 2.60-2.39 (m, 1H), 2.00-1.64 (m, 5H), 1.46 (s, 9H).

### Step F: (2S)-8-Aminooctahydroindolizin-2-ol hydrochloride

To a solution of tert-butyl ((2S)-2-hydroxyoctahydroindolizin-8-yl)carbamate hydrochloride (250.0 mg, 0.85 mmol, 1.0 eq) in 1,4-dioxane (1 mL) was added HCl/dioxane (2.19 mL, 8.75 mmol, 10.25 eq) and stirring was continued at 20° C. for 2 hours. The above reaction mixture was concentrated under reduced pressure to afford the title compound (250 mg, crude product) as yellow oil. ¹H NMR (CD₃OD, 400 MHz) δ [ppm]: 4.44-4.34 (m, 1H), 3.77-3.67 (m, 1H), 3.58-3.42 (m, 1H), 3.20-3.05 (m, 2H), 2.97-2.95 (m, 2H), 1.86-1.63 (m, 4H), 1.58-1.42 (m, 2H).

### Step G: (2S)-8-((5-Bromooxazolo[4,5-b]pyridin-2-yl)amino)octahydroindolizin-2-ol 2,2,2-trifluoroacetate

To a solution of (2S)-8-aminooctahydroindolizin-2-ol hydrochloride (250.0 mg, 1.3 mmol, 1.0 eq) in 1,4-dioxane (2 mL) was added DIPEA (836.84 mg, 6.49 mmol, 5.0 eq). Subsequently a solution of 5-bromo-2-chlorooxazolo[4,5-b]pyridine (272.59 mg, 1.17 mmol, 0.9 eq) in 1,4-dioxane (2 mL) was added dropwise at 0° C., then the above reaction mixture was stirred at 20° C. for 2 hours under nitrogen. The above reaction solution was cooled to room temperature, filtered and the filtrate was concentrated under reduced pressure. The crude product was purified by reversed phase flash (0.1% TFA, water-MeCN), then the solvent was removed by lyophilization to afford the title compound (50.0 mg, 8% yield) as yellow oil. LCMS: m/z 355.0 [M+H]+, ESI pos.

### Step H: 3-Hydroxy-5-methyl-4-[2-[[(2)-2-hydrox-1,2,3,5,6,7,8,8a-octahydroindolizin-8-yl]amino]oxazolo[4,5-b]pyridin-5-yl]benzonitrile;2,2,2-trifluoroacetic acid

To a solution of (2S)-8-((5-bromooxazolo[4,5-b]pyridin-2-yl)amino)octahydroindolizin-2-ol 2,2,2-trifluoroacetate (20.0 mg, 0.04 mmol, 1.0 eq) in 1,4-dioxane (1 mL) and water (0.2 mL) was added (4-cyano-2-hydroxy-6-methylphenyl)boronic acid (7.6 mg, 0.04 mmol, 1.0 eq), CsF (16.0 mg, 0.11 mmol, 2.46 eq) and XPhos Pd G3 (7.0 mg, 0.01 mmol, 0.19 eq). The reaction mixture was stirred at 95° C. for 2 hours under nitrogen, then was cooled to room temperature, filtered and the filtrate was concentrated under reduced pressure. The crude product was purified by reversed phase flash (0.10% TFA, water-MeCN). The solvent was removed by lyophilization and the yellow solid was purified by prep-HPLC (Column YMC Triart C₁₈ 150×25 mm×5 μm; condition water(TFA)-MeCN Begin B 22End B 42 Gradient Time(min) 10 100% B Hold Time(min) 2; Flowrate (mL/min) 25) to afford the title compound (1.93 mg, 8% yield) as yellow gum. LCMS: m/z 406.1 [M+H]⁺, ESI pos.

### Example 116

### 2-[7-(Dimethylamino)-2-[[(3R)-1-ethyl-3-piperidyl]amino]oxazolo[4,5-b]pyridin-5-yl]-3-methyl-5-(trifluoromethyl)phenol

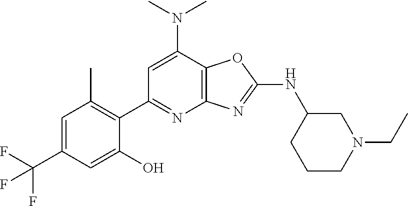 [SMILES: CCN1CCCC(Nc2nc3nc(-c4c(C)cc(C(F)(F)F)cc4O)cc(N(C)C)c3o2)C1]

### Step A: tert-Butyl (3R)-3-[[7-bromo-5-[2-methoxy-6-methyl-4-(trifluoromethyl)-phenyl]oxazolo[4,5-b]pyridin-2-yl]amino]piperidine-1-carboxylate

tert-Butyl (3R)-3-[[5-[2-methoxy-6-methyl-4-(trifluoromethyl)phenyl]-4-oxido-oxazolo[4,5-b]pyridin-4-ium-2-yl]amino]piperidine-1-carboxylate (Example 103, step B) (580 mg, 1.11 mmol, 1.0 eq) was dissolved in DMF (20 mL) and oxalyl bromide (0.95 mL, 6.66 mmol, 6.0 eq) was added with vigorous stirring at 0° C. The mixture was stirred at room temperature for 20 min, then 2 N aqueous NaOH (2 mL) was added. The mixture was diluted with EtOAc (50 mL), washed with 10 wt % aqueous LiCl (3×50 mL), dried using a phase separator and concentrated in vacuo. The resulting residue was purified by chromatography on silica gel (24 g column, 0-7% MeOH/DCM) to afford the title compound (250 mg, 31% yield) as a light yellow solid. LCMS m/z 585.1 (⁷⁹Br) [M+H]⁺, ESI pos.

### Step B: tert-Butyl (3R)-3-[[7-(dimethylamino)-5-[2-methoxy-6-methyl-4-(trifluoromethyl)-phenyl]oxazolo[4,5-b]pyridin-2-yl]amino]piperidine-1-carboxylate

Dimethylamine (2 M in THF) (2.0 mL, 4.0 mmol, 38.26 eq), RuPhos Pd G3 (26.23 mg, 0.03 mmol, 0.3 eq), tert-butyl (3R)-3-[[7-bromo-5-[2-methoxy-6-methyl-4-(trifluoromethyl)-phenyl]oxazolo[4,5-b]pyridin-2-yl]amino]piperidine-1-carboxylate (72.0 mg, 0.1 mmol, 1.0 eq) and Cs₂CO₃ (136.3 mg, 0.42 mmol, 4.0 eq) were combined in a sealed vial. This was heated to 90° C. in a microwave reactor for 2 h. The reaction mixture was dry loaded onto silica and purified by flash chromatography (silica gel, 0-10% (0.7 N ammonia in MeOH)/DCM) to afford the title compound (62.0 mg, 90% yield) as a brown gum. LCMS m/z 550.5 [M+H]⁺, ESI pos.

### Step C: 2-[7-(Dimethylamino)-2-[[(3R)-1-ethyl-3-piperidyl]amino]oxazolo[4,5-b]pyridin-5-yl]-3-methyl-5-(trifluoromethyl)phenol

tert-Butyl (3R)-3-[[7-(dimethylamino)-5-[2-methoxy-6-methyl-4-(trifluoromethyl)phenyl]-oxazolo[4,5-b]pyridin-2-yl]amino]piperidine-1-carboxylate (70.0 mg, 0.11 mmol, 1.0 eq) was dissolved in DCM (4 mL) and boron tribromide (1 M in DCM) (0.53 mL, 0.53 mmol, 5.0 eq) was added. The mixture was stirred at room temperature for 2 h, then concentrated in vacuo. The resulting residue was dissolved in 0.7 N ammonia in MeOH (5 mL), then concentrated in vacuo. The resulting residue was dissolved in EtOH (4 mL) and one drop of AcOH was added, followed by acetaldehyde (0.02 mL, 0.42 mmol, 4.0 eq). The reaction was stirred for 1 h, then STAB (89.62 mg, 0.42 mmol, 4.0 eq) was added. The reaction was stirred for 1 h, then wet MeOH (5 mL) was added, and the mixture left to stand for 16 h. The reaction mixture was dry-loaded onto silica and purified by flash chromatography on silica gel (4 g column, 0-15% (0.7 N ammonia in MeOH)/DCM) to afford an orange solid (40 mg). This was dissolved in DMSO (1 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% ammoniac in water-MeCN gradient over 12.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 40% MeCN; 0.5-10.5 min, ramped from 40% MeCN to 70% MeCN; 10.5-10.6 min, ramped from 70% MeCN to 100% MeCN; 10.6-12.5 min, held at 100% MeCN. This afforded the title compound (7.2 mg, 15% yield) as an off-white solid. LCMS m/z 464.5 [M+H]+, ESI pos.

### Example 117

### 2-[[(3R)-1-Ethyl-3-piperidyl]amino]-5-[2-hydroxy-6-methyl-4 (trifluoromethyl)phenyl]-oxazolo[4,5-b]pyridine-7-carbonitrile

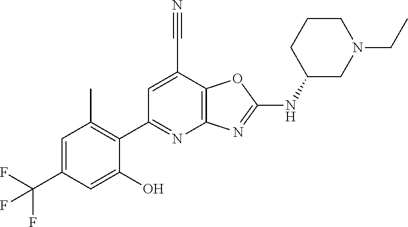 [SMILES: CCN1CCC[C@@H](Nc2nc3nc(-c4c(C)cc(C(F)(F)F)cc4O)cc(C#N)c3o2)C1]

Potassium ferrocyanide trihydrate (74.3 mg, 0.18 mmol, 4.0 eq), 2-[7-chloro-2-[[(3R)-1-ethyl-3-piperidyl]amino]oxazolo[4,5-b]pyridin-5-yl]-3-methyl-5-(trifluoromethyl)phenol (Example 101, step A) (25.0 mg, 0.04 mmol, 1.0 eq), Pd-117 (CAS #205319-06-8, 23.6 mg, 0.03 mmol, 0.75 eq) and KOAc (8.62 mg, 0.13 mmol, 3.0 eq) were suspended in 1,4-dioxane (1 mL) and water (0.5 mL) was added. The reaction mixture was degassed (N2, 5 min), then stirred at 90° C. for 6 h. The reaction mixture was concentrated in vacuo and the resulting residue dissolved in 20% MeOH in DCM (20 mL), then filtered through a plug of silica. The filtrate was concentrated in vacuo. The resulting residue was dissolved in DMSO (1 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min⁻¹ eluting with a 0.3% Ammonia in water-MeCN gradient over 12.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 25% MeCN; 0.5-10.5 min, ramped from 25% MeCN to 55% MeCN; 10.5-10.6 min, ramped from 55% MeCN to 100% MeCN; 10.6-12.5 min, held at 100% MeCN. This afforded the title compound (2.7 mg, 13% yield) as a light-yellow solid. LCMS m/z 446.4 [M+H⁺, ESI pos.

### Example 118a and 118b

### 5-chloro-2-[2-[[(3R)-1-[[rac trans-2-hydroxycyclobutyl]methyl]-3-piperidyl]amino]oxazolo-[4,5-b]pyridin-5-yl]-3-methyl-phenol or 5-chloro-2-[2-[[(3R)-1-[[rac cis-2-hydroxy-cyclobutyl]methyl]-3-piperidyl]amino]oxazolo[4,5-b]pyridin-5-yl]-3-methyl-phenol and 5-chloro-2-[2-[[(3R)-1-[[rac trans-2-hydroxycyclobutyl]methyl]-3-piperidyl]amino]oxazolo-[4,5-b]pyridin-5-yl]-3-methyl-phenol or 5-chloro-2-[2-[[(3R)-1-[[rac cis-2-hydroxy-cyclobutyl]methyl]-3-piperidyl]amino]oxazolo[4,5-b]pyridin-5-yl]-3-methyl-phenol

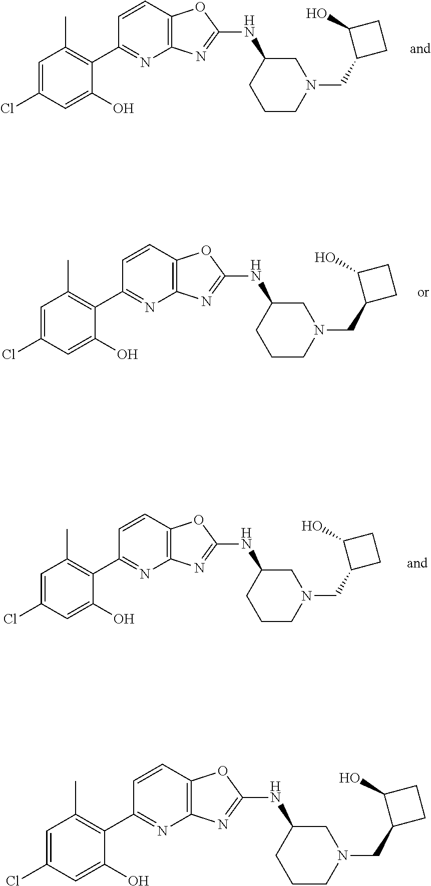

### Step A: 2-[[(3R)-3-[(5-bromooxazolo[4,5-b]pyridin-2-yl)amino]-1-piperidyl]methyl]cyclo-butanone

A solution of (2-oxocyclobutyl)methyl methanesulfonate (150 mg, 0.84 mmol, 1.0 eq) in MeCN (3 mL) was added dropwise to a suspension of 5-bromo-N-[(3R)-3-piperidyl]oxazolo[4,5-b]pyridin-2-amine;dihydrochloride (Example 75, step B) (307 mg, 0.83 mmol, 1.0 eq) and cesium carbonate (1.11 g, 3.39 mmol, 4.1 eq) in MeCN (5 mL) and stirring was continued at r.t. overnight (16 h). Then, cesium carbonate (270 mg, 0.83 mmol, 1.0 eq) and (2-oxocyclobutyl)methyl methanesulfonate (50.0 mg, 0.28 mmol, 0.34 eq) in MeCN (1 mL) was added and the reaction was left to stir for an additional hour. The reaction was concentrated under reduced pressure and loaded onto silica. The crude reaction mixture was purified by column chromatography (silica, 40 g, 0-10% MeOH (0.7M NH₃):EtOAc) to afford the title compound (167.0 mg, 52% yield) as a colourless gum. LCMS m/z: 379.2; 381.2 (br isotopes) [M+H]⁺, ESI pos.

### Step B: 2-[[(3R)-3-[[5-(4-Chloro-2-hydroxy-6-methyl-phenyl)oxazolo[4,5-b]pyridin-2-yl]amino]-1-piperidyl]methyl]cyclobutanone

A solution of aforementioned 2-[[(3R)-3-[(5-bromooxazolo[4,5-b]pyridin-2-yl)amino]-1-piperidyl]methyl]-cyclobutanone (162 mg, 0.43 mmol, 1.0 eq), (4-chloro-2-hydroxy-6-methyl-phenyl)boronic acid (93.0 mg, 0.5 mmol, 1.17 eq) and potassium carbonate (120.0 mg, 0.87 mmol, 2.03 eq) in 1,4-dioxane (3 mL)) and water (1 mL) was sparged with N₂ for 10 min. [1,1′-bis(diphenylphosphino)ferrocene]-dichloropalladium(II) (16.0 mg, 0.02 mmol, 0.05 eq) was added and the reaction mixture was heated to 90° C. for 18 h. The reaction was cooled to r.t. (4-chloro-2-hydroxy-6-methyl-phenyl)boronic acid (16.0 mg, 0.09 mmol, 0.2 eq) and potassium carbonate (30.0 mg, 0.22 mmol, 0.51 eq) was added followed by [1,1′-bis(diphenylphosphino)ferrocene]dichloropalladium(II) (6.0 mg, 0.01 mmol, 0.02 eq) after sparging for 5 min. The reaction was heated to 90° C. for a further 1 h. The reaction mixture was cooled and concentrated under reduced pressure directly onto silica. The crude reaction mixture was purified by column chromatography (on silica, 0-15% MeOH:EtOAc) to afford the title compound (102 mg, 38% yield) as a brown solid. LCMS m/z 441.3; 443.4 (Cl isotopes) [M+H]+, ESI pos.

### Step C: 5-Chloro-2-(2-(((3R)-1-((2-hydroxycyclobutyl)methyl)piperidin-3-yl)amino)oxazolo[4,5-b]pyridin-5-yl)-3-methylphenol

To a stirred solution of 2-[[(3R)-3-[[5-(4-chloro-2-hydroxy-6-methyl-phenyl)oxazolo[4,5-b]pyridin-2-yl]amino]-1-piperidyl]methyl]cyclobutanone (95.0 mg, 0.22 mmol, 1.0 eq) in THF (4 mL) at 0° C. was added sodium borohydride (25.0 mg, 0.66 mmol, 3.07 eq) and the reaction was stirred at rt for 30 mins. The reaction was quenched with 1 M HCl (˜500 μL) and the reaction was left to stir for 15 min. Then MeOH (0.5 mL) was added, and the reaction mixture was stirred at 50° C. for 4 hrs and then at rt for 16 h. The reaction mixture was concentrated under reduced pressure and the crude product was purified by reversed phase preparative HPLC (Waters XBridge BEH C18 ODB prep column, 130 Å, 5 μm, 30 mm×100 mm, flow rate 40 mL min; gradient information: 0.0-0.5 min, 25% MeCN; 0.5-10.5 min, ramped from 25% MeCN to 55% MeCN; 10.5-10.6 min, ramped from 55% MeCN to 100% MeCN; 10.6-12.5 min, held at 100% MeCN) to afford 118b (9.57 mg, 10% yield) and 118a (1.81 mg, 1.9% yield). LCMS (118b) m/z 433.4; 345.4 (Cl isotopes) [M+H]⁺, ESI pos. ¹H NMR (500 MHz, MeOD) δ [ppm]: 7.67 (dd, 1H), 7.01 (d, 1H), 6.82 (d, 1H), 6.78 (d, 1H), 4.04-3.95 (m, 1H), 3.80-3.70 (m, 1H), 3.16-3.03 (m, 1H), 2.82-2.71 (m, 1H), 2.70-2.60 (m, 1H), 2.51-2.44 (m, 1H), 2.44-2.34 (m, 1H), 2.34-2.22 (m, 2H), 2.22-2.15 (m, 1H), 2.11 (s, 3H), 2.06-2.01 (m, 1H), 1.93-1.81 (m, 2H), 1.81-1.75 (m, 1H), 1.75-1.66 (m, 1H), 1.58-1.45 (m, 1H), 1.24-1.13 (m, 1H). LCMS (118a) m/z 433.4; 345.4 (Cl isotopes) [M+H]⁺, ESI pos. ¹H NMR (500 MHz, MeOD) δ [ppm]: 7.67 (d, 1H), 7.01 (d, 1H), 6.82 (dd, 1H), 6.78 (d, 1H), 4.44-4.34 (m, 1H), 4.01-3.95 (m, 1H), 3.14-3.04 (m, 1H), 2.98-2.91 (m, 1H), 2.86-2.69 (m, 2H), 2.59-2.51 (m, 1H), 2.34-2.18 (m, 3H), 2.11 (s, 3H), 2.09-1.98 (m, 2H), 1.91-1.80 (m, 2H), 1.75-1.60 (m, 2H), 1.57-1.47 (m, 1H).

### Example 119

### 2-[2-[[(3R)-1-Ethyl-3-piperidyl]amino]oxazolo[4,5-b]pyridin-5-yl]-3-hydroxy-benzonitrile

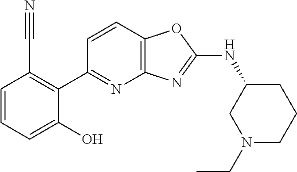 [SMILES: CCN1CCC[C@@H](Nc2nc3nc(-c4c(O)cccc4C#N)ccc3o2)C1]

### Step A: 2-[2-[[(3R)-1-Ethyl-3-piperidyl]amino]oxazolo[4,5-b]pyridin-5-yl]-3-methoxy-benzonitrile

A mixture of 2-(5,5-dimethyl-1,3,2-dioxaborinan-2-yl)-3-methoxy-benzonitrile (CAS 3 214360-47-1, 50.0 mg, 0.2 mmol, 1.1 eq), 5-bromo-N-[(3R)-1-ethyl-3-piperidyl]oxazolo[4,5-b]pyridin-2-amine (Example 21, step H) (60.0 mg, 0.18 mmol, 1.0 eq), cesium carbonate (180.0 mg, 0.55 mmol, 3.0 eq) and XPhosPdG3 (16.0 mg, 0.02 mmol, 0.1 eq) in MeCN (1.8 mL) and water (0.2 mL) was degassed with N₂ for 5 mins, then was heated to 60° C. and was stirred for ˜16 h. The reaction was allowed to cool to rt, then was concentrated in vacuo. The crude material was purified by column chromatography on silica gel (0-10% MeOH(NH3)/DCM) to give the title compound (23.2 mg, 26% yield) as a yellow gum. LCMS m/z: 378.4 [M+H]⁺, ESI pos.

The reaction was repeated once more, starting with 80 mg of 5-bromo-N-[(3R)-1-ethyl-3-piperidyl]oxazolo[4,5-b]pyridin-2-amine.

### Step B: 2-[2-[[(3R)-1-Ethyl-3-piperidyl]amino]oxazolo[4,5-b]pyridin-5-yl]-3-hydroxy-benzonitrile

BBr₃ (0.95 mL, 0.95 mmol, 10 eq) was added dropwise to a stirred solution of 2-[2-[[(3R)-1-ethyl-3-piperidyl]amino]oxazolo[4,5-b]pyridin-5-yl]-3-methoxy-benzonitrile (48.0 mg, 0.09-mmol, 1.0 eq) and tetrabutylammonium iodide (120.0 mg, 0.32 mmol, 3.5 eq) in DCM (3 mL) at 0° C. and the reaction was allowed to rt. The reaction was stirred for a further ˜16 h, then was quenched by adding dropwise to a stirred solution of NH₃ in MeOH (50 mL, 7M) at 0° C. The solvent was concentrated in vacuo, and the resulting residue was taken up in DCM (20 mL) and water (20 mL). The aqueous phase was adjusted to ˜pH 12 with NaOH (2M aq.) and the organic phase was separated. The aqueous phase was then adjusted ˜pH 7 with HCl (1M aq.) and then was extracted again with DCM (3×20 mL). The combined organic layers were dried with MgSO₄, concentrated in vacuo and then purified by column chromatography on silica gel (12 g cartridge, 0-10% MeOH (0.7M NH₃)/DCM) to give the title compound (9.2 mg, 27% yield) as an off-white solid. LCMS m/z: 364.3 [M+H]⁺, ESI pos.

### Example 120

### 3-Methyl-2-[2-[(2-methyl-2-azabicyclo[2.2.2]octan-4-yl)amino]oxazolo[4,5-b]pyridin-5-yl]-5-(trifluoromethyl)phenol

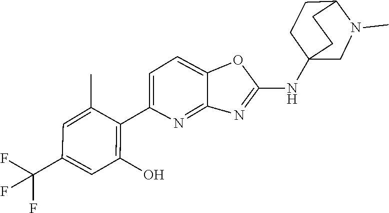 [SMILES: Cc1cc(C(F)(F)F)cc(O)c1-c1ccc2oc(NC34CCC(CC3)N(C)C4)nc2n1]

### Step A: 4-[(5-Chlorooxazolo[4,5-b]pyridin-2-yl)amino]-2-azabicyclo[2.2.2]octane-2-carboxylic acid tert-butyl ester

To a mixture of 5-chloro-2-(methylthio)oxazolo[4,5-b]pyridine (150 mg, 0.748 mmol, 1.00 eq) in 1,4-dioxane (0.91 mL) was added 4-amino-2-azabicyclo[2.2.2]octane-2-carboxylic acid tert-butyl ester (186.11 mg, 0.822 mmol, 1.10 eq) followed by triethylamine (84.72 mg, 116.7 μL, 0.837 mmol, 1.12 eq). The brown solution was stirred at 90° C. overnight. Afterwards, 4-amino-2-azabicyclo[2.2.2]octane-2-carboxylic acid tert-butyl ester (64 mg, 0.234 mmol, 0.314 eq) and triethylamine (84.72 mg, 116.7 uL, 0.837 mmol, 1.120 eq) were added to the reaction mixture and stirring was continued at 100° C. for 4 hours. The solvent was evaporated and the residue was dissolved in NMP (0.91 mL). To the reaction was added triethylamine (84.7 mg, 117 μL, 0.837 mmol, 1.12 eq) and it was stirred for two hours at 140° C. Again, triethylamine (117 uL, 0.837 mmol, 1.12 eq) was added to the reaction mixture and it was stirred at 140° C. overnight. After cooling to r.t the reaction mixture was extracted with ethyl acetate (˜20 mL), and 10% aq. LiCl (˜5 mL) solution. The aqueous layer was back extracted with ethyl acetate (˜20 mL). The organic layers were washed with water (˜10 mL) and brine (˜10 mL). The combined organic layers were dried over anhydrous sodium sulfate, filtered and concentrated in vacuo. The crude was purified by flash chromatography (silica gel, 12 g, gradient 0% to 10% MeOH in DCM) to afford the title compound (63 mg, 59%) as a white solid. LCMS m/z: 379.2 [M+H]⁺, ESI pos.

### Step B: 2-Azabicyclo[2.2.2]octan-4-yl-(5-chlorooxazolo[4,5-b]pyridin-2-yl)amine

According to GP3 the title compound was obtained as a white solid (30 mg, 53% yield). LCMS m/z: 279.1 [M+H]⁺, ESI pos.

### Step C: 5-Chlorooxazolo[4,5-b]pyridin-2-yl)-(2-methyl-2-azabicyclo[2.2.2]octan-4-yl)amine

To a mixture of 2-azabicyclo[2.2.2]octan-4-yl-(5-chlorooxazolo[4,5-b]pyridin-2-yl)amine (30 mg, 0.11 mmol, 1.0 eq) in 1,2-dichloroethane (1.16 mL) was added triethylamine (16.77 mg, 23.1 μL, 0.166 mmol, 1.54 eq) and stirred for 5 minutes at r.t. Then, formaldehyde, 37% aqueous solution (11.6 mg, 10.66 μL, 0.143 mmol, 1.33 eq) was added followed by sodium triacetoxyborohydride (68.6 mg, 0.324 mmol, 3.01 eq) and stirring was continued at r.t. for 3 hours. Again, formaldehyde, 37% aqueous solution (11.6 mg, 10.7 μL, 0.14 mmol, 1.3 eq) was added. The reaction mixture was stirred at r.t. for 2 hours. The reaction mixture was extracted with DCM (˜30 mL) and sat. aq. NaHCO₃-solution (˜5 mL). The aqueous layer was back extracted twice with DCM (˜30 mL). The combined organic layers were dried over anhydrous sodium sulfate, filtered and concentrated in vacuo. The crude product was purified by flash chromatography (silica gel, 4 g, DCM/MeOH gradient 0% to 20% methanol+1% NH₃) to afford the title compound (19 mg, 60%) as off-white solid. LCMS m/z: 293.1 [M+H]⁺, ESI pos.

### Step D: 3-Methyl-2-[2-[(2-methyl-2-azabicyclo[2.2.2]octan-4-yl)amino]oxazolo[4,5-b]pyridin-5-yl]-5-(trifluoromethyl)phenol

According to GP2b 5-chlorooxazolo[4,5-b]pyridin-2-yl)-(2-methyl-2-azabicyclo[2.2.2]octan-4-yl)amine (16 mg, 54.7 umol, 1.0 eq) and 3-methyl-2-(4,4,5,5-tetramethyl-1,3,2-dioxaborolan-2-yl)-5-(trifluoromethyl)phenol (24 mg, 19.8 μL, 79.4 μmol, 1.45 eq) were reacted to afford the title compound as a yellow solid (7.3 mg, 28% yield). LCMS m/z: 431.2[M−H]⁻, ESI neg.

### Example 121

### 5-(Difluoromethyl)-3-methyl-2-[2-[[(3R)-1-methyl-3-piperidyl]amino]oxazolo[4,5-b]pyridin-5-yl]phenol

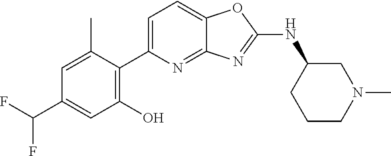 [SMILES: Cc1cc(C(F)F)cc(O)c1-c1ccc2oc(N[C@@H]3CCCN(C)C3)nc2n1]

Similarly to GP2a, 5-bromo-N-[(3R)-1-methyl-3-piperidyl]oxazolo[4,5-b]pyridin-2-amine; 2,2,2-trifluoroacetic acid (Example 30, step H) (143.7 mg, 0.34 mmol, 1.2 eq) and 5-(difluoromethyl)-3-methyl-2-(4,4,5,5-tetramethyl-1,3,2-dioxaborolan-2-yl) phenol (80.0 mg, 0.28 mmol, 1.0 eq) were reacted. The crude was purified by column chromatography (C₁₈, 0.1% TFA in water/MeCN) and re-purified by prep-HPLC (column: Waters Xbridge 150×25 mm×5 μm; condition: water (NH₄HCO₃)-MeCN; Begin B: 29; End B: 59; Gradient Time(min): 9; 100% B Hold Time(min): 2; FlowRate (ml/min): 25) to yield the title compound (14.49 mg, 0.04 mmol, 13% yield) as yellow solid. LCMS: m/z 389.3 [M+H]+, ESI pos.

### Example 122

### 5-(Difluoromethyl)-2-[2-[[(3R)-1-ethyl-3-piperidyl]amino]oxazolo[4,5-b]pyridin-5-yl]-3-methyl-phenol;2,2,2-trifluoroacetic acid

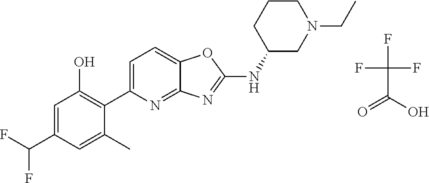 [SMILES: CCN1CCC[C@@H](Nc2nc3nc(-c4c(C)cc(C(F)F)cc4O)ccc3o2)C1 ; O=C(O)C(F)(F)F]

According to GP2a 5-bromo-N-[(3R)-1-ethyl-3-piperidyl]oxazolo[4,5-b]pyridin-2-amine (Example 21, step H) (41.2 mg, 0.13 mmol, 1.2 eq) and 5-(difluoromethyl)-3-methyl-2-(4,4,5,5-tetramethyl-1,3,2-dioxaborolan-2-yl)phenol (30.0 mg, 0.11 mmol, 1.0 eq) were reacted. The crude was purified by reversed phase flash (0.1% TFA water/ACN condition, 60 g C₁₈ cartridge, detection in 220 nm and 254 nm) to yield the title compound (4.38 mg, 0.01 mmol, 8% yield) as a yellow solid. LCMS: m/z 403.2 [M+H]+, ESI pos.

### Example 123

### 2-[2-[[(3R)-1-Ethyl-3-piperidyl]amino]oxazolo[4,5-b]pyridin-5-yl]-3,5-dimethyl-phenol

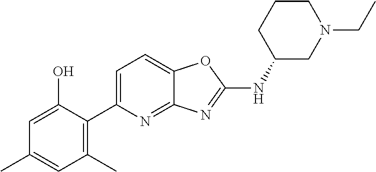

### Step A: 2-Iodo-3,5-dimethyl-phenol

To a mixture of 3,5-dimethylphenol (5.0 g, 40.93 mmol, 1.0 eq, CAS: 108-68-9) in toluene (100 mL) was added NaH (3.27 g, 81.86 mmol, 2.0 eq 60%) at 0° C. under N₂, stirred for 30 mins, then I₂ (8.31 g, 32.74 mmol, 0.8 eq) was added and stirred for 15.5 hours at 25° C. The mixture was poured into 1N HCl (200 mL) and extracted with ethyl acetate (60 mL×3). The combined organic layers were washed with brine (50 mL), dried over anhydrous sodium sulfate, filtered, and the filtrate was concentrated under reduced pressure. The residue was purified by silica gel column chromatography (petroleum ether:ethyl acetate=1:0 to 10:1) to afford the title compound (1.10 g, 11% yield) as a white solid. LCMS: m/z 246.9 [M−H]−, ESI neg.

### Step B: 1-Benzyloxy-2-iodo-3,5-dimethyl-benzene

To a solution of 2-iodo-3,5-dimethyl-phenol (1.1 g, 4.43 mmol, 1.0 eq) in DMF (20 mL) was added K₂CO₃ (1.23 g, 8.87 mmol, 2.0 eq) portion wise, followed by dropwise addition of benzyl bromide (0.79 mL, 6.65 mmol, 1.5 eq). The reaction mixture was stirred at 25° C. for 2 hrs under nitrogen atmosphere. Afterwards, the reaction was quenched with water (50 mL), extracted with EtOAc (100 mL×3), washed with brine (20 mL). The organic layer was dried over Na₂SO₄, filtrated and the filtrate was concentrated under reduced pressure. The residue was purified by silica gel column chromatography (petroleum ether:ethyl acetate=1:0 to 5:1) to afford the title compound (1.3 g, 87% yield) as colourless oil. ¹H NMR (400 MHz, CD₃OD) δ [ppm]: 7.52 (d, 2H), 7.39-7.35 (m, 2H), 7.32-7.25 (m, 1H), 6.76 (s, 1H), 6.62 (s, 1H), 5.19 (s, 1H), 5.11 (s, 2H), 2.40 (s, 3H), 2.26 (s, 3H).

### Step C: 2-(2-Benzyloxy-4,6-dimethyl-phenyl)-4,4,5,5-tetramethyl-1,3,2-dioxaborolane

To a mixture of 1-benzyloxy-2-iodo-3,5-dimethyl-benzene (650.0 mg, 1.92 mmol, 1.0 eq) and 2-isopropoxy-4,4,5,5-tetramethyl-1,3,2-dioxaborolane (643.71 mg, 3.46 mmol, 1.8 eq) in THF (7 mL) and was added dropwise nBuLi (1.15 mL, 2.88 mmol, 1.5 eq) under nitrogen atmosphere and stirred at −60° C. for 30 mins. The mixture was poured into an aq. NH₄Cl solution (30 mL) and extracted with ethyl acetate (30 mL×3). The combined organic layers were washed with brine (20 mL), dried over anhydrous sodium sulfate, filtered, and the filtrate was concentrated under reduced pressure. The residue was purified by silica gel column chromatography (petroleum ether:ethyl acetate=1:0 to 20:1) to give the title compound (500.0 mg, 1.48 mmol, 77% yield) as a colorless oil. ¹H NMR (400 MHz, CD₃OD) δ [ppm]: 7.47 (d, 2H), 7.37-7.28 (m, 3H), 6.62 (d, 2H), 4.99 (s, 2H), 2.28 (d, 6H), 1.27 (s, 12H).

### Step D: (2-Hydroxy-4,6-dimethyl-phenyl)boronic acid and 3,5-dimethyl-2-(4,4,5,5-tetramethyl-1,3,2-dioxaborolan-2-yl)phenol

A mixture of 2-(2-benzyloxy-4,6-dimethyl-phenyl)-4,4,5,5-tetramethyl-1,3,2-dioxaborolane (250.0 mg, 0.74 mmol, 1.0 eq) in EtOAc (3 mL) was degassed and purged with H₂ three times before Pd/C (15 mg) and Pa(OH)₂ (15.0 mg) was added to the mixture in one portion. The mixture was stirred at 25° C. for 2 hours. The mixture was filtered off and the filtrate was concentrated under reduced pressure to afford the title compound (100.0 mg, 0.4 mmol, 55% yield) as yellow oil. ¹H NMR (400 MHz, CD₃OD) δ [ppm]: 6.49 (s, 1H), 6.43 (s, 1H), 4.87 (s, 7H), 2.38 (s, 3H), 2.21 (s, 3H), 2.01 (s, 1H), 1.38 (s, 12H).

### Step E: 2-[2-[[(3R)-1-Ethyl-3-piperidyl]amino]oxazolo[4,5-b]pyridin-5-yl]-3,5-dimethyl-phenol

A mixture of CsF (104.08 mg, 0.75 mmol, 2.5 eq), (2-hydroxy-4,6-dimethyl-phenyl)boronic acid (50.0 mg, 0.3 mmol, 1.0 eq) and 5-bromo-N-[(3R)-1-ethyl-3-piperidyl]oxazolo[4,5-b]-pyridin-2-amine (97.96 mg, 0.3 mmol, 1.0 eq) in 1,4-dioxane (1 mL) and water (0.2 mL) was degassed and purged with N2 three times and Pd(dppf)Cl₂ (22.04 mg, 0.03 mmol, 0.1 eq) was added to the mixture. The mixture was heated under microwave irradiation at 90° C. for 2 hours. Afterwards, the mixture was poured into water (10 mL) and extracted with ethyl acetate (15 mL×3). The combined organic layers were washed with brine (20 mL), dried over anhydrous sodium sulfate, filtered, and the residue was purified by C18 column chromatography (20 g, 0.10% TFA in water/MeCN) to afford the title compound (5.1 mg, 0.01 mmol, 5% yield) as a white solid. LCMS: m/z 367.2. [M+H]+, ESI pos.

### Example 124

### 3-[[5-(4-Chloro-2-hydroxy-6-methyl-phenyl)oxazolo[4,5-b]pyridin-2-yl]amino]-1-ethyl-piperidin-4-ol

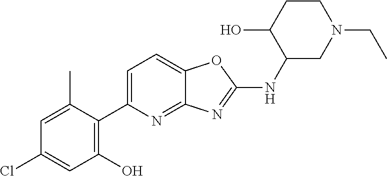

### Step A: 3-[(5-Chlorooxazolo[4,5-b]pyridin-2-yl)amino]-1-ethyl-piperidin-4-ol

To a dark orange-brown mixture of 5-chloro-2-(methylthio)oxazolo[4,5-b]pyridine (300 mg, 1.4 mmol, 1.0 eq) in 1,4-dioxane (1.5 mL) and NMP (1.5 mL) was added under stirring 3-amino-1-ethyl-piperidin-4-ol;hydrochloride (308 mg, 1.7 mmol, 1.2 eq) followed by triethylamine (359.3 mg, 495 μL, 3.55 mmol, 2.5 eq) at r.t. The brown solution was stirred at 100° C. for 16 hrs and then heated to 150° C. for 4 hrs. To the dark brown reaction mixture was added under stirring more 3-amino-1-ethyl-piperidin-4-ol;hydrochloride (100 mg, 0.553 mmol, 0.4 eq) and triethylamine (72.6 mg, 100 μL, 0.72 mmol, 0.501 eq) at r.t. and the mixture heated to 150° C. for 6 hrs. The reaction mixture was quenched with water (10 mL) and extracted with ethylacetate twice (2×30 mL). The organic layers were washed with water (30 mL) and brine (30 mL). The combined organic extracts were dried over anhydrous sodium sulfate, filtered off and concentrated in vacuo. The crude product was purified by flash chromatography (silica gel, 0%-90% DCM:MeOH:NH₄OH (v/v) 110:10:1 in DCM) to afford the title compound (80 mg, 19% yield) as dark brown solid. LCMS m/z: 297.1 [M+H]⁺, ESI pos.

### Step B: 3-[[5-(4-Chloro-2-hydroxy-6-methyl-phenyl)oxazolo[4,5-b]pyridin-2-yl]amino]-1-ethyl-piperidin-4-ol

Following GP2a 3-[(5-chlorooxazolo[4,5-b]pyridin-2-yl)amino]-1-ethyl-piperidin-4-ol (80 mg, 0.27 mmol, 1.0 eq) was reacted with (4-chloro-2-hydroxy-6-methyl-phenyl)boronic acid (79.6 mg, 0.43 mmol, 1.6 eq) to afford the title compound as a white solid (20 mg, 19% yield). LCMS m/z: 401.2; 403.2 (Cl isotopes) [M−H]−, ESI neg.

### Example 125

### 2-[2-[[(3R)-1-Ethyl-3-piperidyl]amino]oxazolo[4,5-b]pyridin-5-yl]-3-methyl-phenol

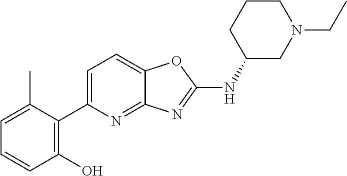

Following to GP2a, 5-chloro-N-[(3R)-1-ethyl-3-piperidyl]oxazolo[4,5-b]pyridin-2-amine (Example 14, step 1) (60 mg, 0.21 mmol, 1.0 eq) and (2-hydroxy-6-methyl-phenyl)boronic acid (52 mg, 0.34 mmol, 1.6 eq) were reacted to afford the title compound (26 mg, 35% yield) as a white amorph freeze-dried solid. LCMS m/z: 353.2 [M+H]⁺, ESI pos.

### Example 126

### 5-Fluoro-2-[2-[[(3R)-1-methyl-3-piperidyl]amino]oxazolo[4,5-b]pyridin-5-yl]-3-(trifluoromethyl)phenol;2,2,2-trifluoroacetic acid

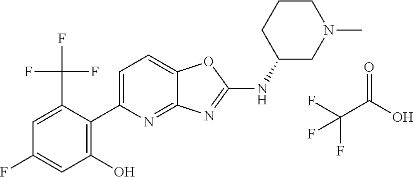

### Step A: 1-(Benzyloxy)-2-bromo-5-fluoro-3-(trifluoromethyl)benzene

To a solution of 2-bromo-5-fluoro-3-(trifluoromethyl)phenol (500.0 mg, 1.93 mmol, 1.0 eq) in DMF (5 mL) was added K₂CO₃ (533.6 mg, 3.86 mmol, 2.0 eq), followed by addition of BnBr (495.3 mg, 2.9 mmol, 1.5 eq) and the reaction mixture was stirred at 25° C. for 2 hrs under nitrogen atmosphere. Afterwards, the reaction mixture was quenched with aq. 1N HCl (20 mL), extracted with ethyl acetate (100 mL×3), washed with brine (20 mL). The combined organic layers were dried over anhydrous Na₂SO₄, filtered and the filtrate was concentrated under reduced pressure. The residue was purified by silica gel chromatography (petroleum ether) to give the title compound (550.0 mg, 72% yield) as a white solid. ¹H NMR (400 MHz, DMSO-d₆) δ [ppm]: 7.56-7.50 (m, 1H), 7.48-7.44 (m, 2H), 7.42-7.40 (m, 2H), 7.38-7.36 (m, 2H), 5.30 (s, 2H).

### Step B: 2-(2-(Benzyloxy)-4-fluoro-6-(trifluoromethyl)phenyl)4,4,5,5-tetramethyl-1,3,2-dioxaborolane

To a solution of 1-benzyloxy-2-bromo-5-fluoro-3-(trifluoromethyl)benzene (300.0 mg, 0.86 mmol, 1.0 eq) and 2-isopropoxy-4,4,5,5-tetramethyl-1,3,2-dioxaborolane (319.76 mg, 1.72 mmol, 2.0 eq) in THF (5 mL) was added n-BuLi (0.62 mL, 1.55 mmol, 1.8 eq) at −60° C., then the reaction mixture was stirred at this temperature for 1 hour under nitrogen atmosphere. The reaction mixture was quenched with a saturated aqueous solution of NH₄Cl (30 mL), extracted with ethyl acetate (100 mL×3) and washed with brine (20 mL). The combined organic layers were dried over anhydrous Na₂SO₄, filtered and the filtrate was concentrated under reduced pressure. The residue was purified by silica gel chromatography (petroleum ether:ethyl acetate=10:0 to 10:1) to give the title compound (60.0 mg, 18% yield) as a white solid. ¹H NMR (400 MHz, CDCl₃) δ [ppm] 7.44-7.36 (m, 5H), 6.95 (d, 1H), 6.78 (d, 1H), 5.04 (s, 2H), 1.28 (s, 12H).

### Step C: 5-Fluoro-2-(4,4,5,5-tetramethyl-1,3,2-dioxaborolan-2-yl)-3-(trifluoromethyl)phenol

To a mixture of 2-(2-(benzyloxy)-4-fluoro-6-(trifluoromethyl)phenyl)-4,4,5,5-tetramethyl-1,3,2-dioxaborolane (40.0 mg, 0.1 mmol, 1.0 eq) in ethyl acetate (2 mL) was added Pd/C (10.0 mg, 10%) and the reaction was stirred at r.t. for 1 h under H₂ at 1100 mmHg. The reaction mixture was filtered and the filtrate was concentrated under vacuum to the title compound (30.0 mg, 97% yield) as a yellow solid. LCMS: m/z 305.0 [M−H]−, ESI neg.

### Step D: 5-Fluoro-2-[2-[[(3R)-1-methyl-3-piperidyl]amino]oxazolo[4,5-b]pyridin-5-yl]-3-(trifluoromethyl)phenol;2,2,2-trifluoroacetic acid

To a solution of 5-bromo-N-[(3R)-1-methyl-3-piperidyl]oxazolo[4,5-b]pyridin-2-amine; 2,2,2-trifluoroacetic acid (Example 30, step H) (40.0 mg, 0.09 mmol, 1.0 eq), 5-fluoro-2-(4,4,5,5-tetra methyl-1,3,2-dioxaborolan-2-yl)-3-(trifluoromethyl)phenol (25.91 mg, 0.08 mmol, 0.9 eq) and K₂CO₃ (38.95 mg, 0.28 mmol, 3.0 eq) in 1,4-dioxane (1 mL)/water (0.2 mL) was added Pd(dppf)Cl₂ (6.88 mg, 0.01 mmol, 0.1 eq) under nitrogen atmosphere and the reaction was stirred for 2 hours at 95° C. The mixture was cooled to 25° C., filtered and the filtrate was concentrated in vacuum to give the crude product which was purified by column chromatography (C₁₈, 0.1% TFA in water/MeCN) to afford the title compound (5.23 mg, 10% yield) as a yellow solid. LCMS: m/z 411.1 [M+H]+, ESI pos.

### Example 127

### 5-Chloro-2-[6-Chloro-2-[[(3R)-1-ethyl-3-piperidyl]amino]oxazolo[4,5-b]pyridin-5-yl]phenol

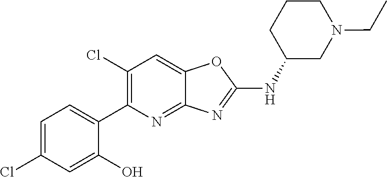 [SMILES: CCN1CCC[C@@H](Nc2nc3nc(-c4ccc(Cl)cc4O)c(Cl)cc3o2)C1]

### Step A: 6-Bromo-5-chloro-3-methoxypyridin-2-amine

The apparatus was dried by heating with a heatgun under vacuum. To a mixture of 6-bromo-3-methoxypyridin-2-amine (800.0 mg, 3.94 mmol, 1.0 eq) in acetic acid (8 mL) was added N-chlorosuccinimide (578.75 mg, 4.33 mmol, 1.1 eq), and the mixture was stirred at 25° C. for 6 hours. The reaction mixture was quenched by water (10 mL), then extracted by EtOAc (30 mL×3), the combined organic phases were washed with brine (40 mL×2), dried over anhydrous sodium sulfate, filtered, and the filtrate was concentrated under reduced pressure. The residue was purified by column (petroleum ether:ethyl acetate=20:1 to 10:1) to afford the title compound (710 mg, 76% yield) as a yellow solid. LCMS: m/z 236.9 [M+H]⁺, ESI pos.

### Step B: 2-(6-Amino-3-chloro-5-methoxypyridin-2-yl)-5-chlorophenol

The apparatus was dried by heating with a heat gun under a vacuum. To a mixture of 6-bromo-5-chloro-3-methoxypyridin-2-amine (700.0 mg, 2.95 mmol, 1.0 eq), 4-chloro-2-hydroxyphenyl boronic acid (558.89 mg, 3.24 mmol, 1.1 eq) and K₂CO₃ (813.54 mg, 5.9 mmol, 2.0 eq) in 1,4-dioxane (10 mL) and water (2 mL) was added Pd(dppf)Cl₂ (215.47 mg, 0.29 mmol, 0.1 eq). The mixture was evacuated and refilled with nitrogen for three times and stirred at 90° C. for 2 hours. After cooling to ambient temperature, the reaction mixture was quenched by addition of water (10 mL), then extracted with EtOAc (10 mL×3) and the combined organic phase were washed brine (20 mL×2), dried over anhydrous sodium sulfate, filtered and the filtrate was concentrated under reduced pressure. The residue was purified by silica gel column chromatography (petroleum ether:ethyl acetate=10:1 to 5:1) to the title compound (600.0 mg, 71% yield) as a yellow solid. LCMS: m/z 284.9 [M+H]+, ESI pos.

### Step C: 2-Amino-5-chloro-6-(4-chloro-2-hydroxyphenyl)pyridin-3-ol

The apparatus was dried by heating with a heat gun under vacuum. To a mixture of 2-(6-amino-3-chloro-5-methoxypyridin-2-yl)-5-chlorophenol (50.0 mg, 0.18 mmol, 1.0 eq) and BBr₃ (439.31 mg, 1.75 mmol, 10.0 eq) was added in DCM (0.5 mL) under nitrogen, stirred at −60° C. for 10 minutes, then the mixture was stirred at 25° C. for 1 h. The reaction mixture was quenched by ice water (2 mL) and neutralized by NH₃H₂O. The mixture was purified by column chromatography (C₁₈, 0.1% NH3H2O in water/MeCN) to yield the title compound (20.0 mg, 42% yield) as a yellow solid. LCMS: m/z 270.9 [M+H]+, ESI pos.

### Step D: 5-Chloro-2-(6-chloro-2-mercaptooxazolo[4,5-b]pyridin-5-yl)phenol

The apparatus was dried by heating with a heat gun under vacuum. To a mixture of 2-amino-5-chloro-6-(4-chloro-2-hydroxyphenyl)pyridin-3-ol (20.0 mg, 0.07 mmol, 1.0 eq) in DMF (3 mL) was added TCDI (19.7 mg, 0.11 mmol, 1.5 eq), and the mixture was stirred at 25° C. for 12 hours. The reaction mixture was quenched by water (10 mL), then extracted with EtOAc (10 mL×3), washed with brine (20 mL×2), dried over anhydrous sodium sulfate, filtered and the filtrate was concentrated under reduce pressure to afford the title compound (20.0 mg, 87% yield) as a yellow solid. LCMS: m/z 312.9 [M+H]⁺, ESI pos.

### Step E: 5-Chloro-2-(2,6-dichlorooxazolo[4,5-b]pyridin-5-yl)phenol

The apparatus was dried by heating with a heat gun under vacuum. To a mixture of 5-chloro-2-(6-chloro-2-mercaptooxazolo[4,5-b]pyridin-5-yl)phenol (20.0 mg, 0.06 mmol, 1.0 eq) in oxalyl chloride (405.32 mg, 3.19 mmol, 50.0 eq) was added DMF (0.47 mg, 0.01 mmol, 0.1 eq), and the mixture was stirred at 50° C. for 1 hour. After cooling to ambient temperature, the reaction mixture was quenched by addition of ice-water (5 mL), then extracted with EtOAc (10 mL×3), washed with brine (15 mL×3), dried over anhydrous sodium sulfate, filtered and the filtrate was concentrated under reduced pressure to afford the title compound (20.0 mg, 0.06 mmol, 99% yield) as a yellow solid. LCMS: m/z 316.7 [M+H]+, ESI pos.

### Step F: 5-Chloro-2-[6-Chloro-2-[[(3R)-1-ethyl-3-piperidyl]amino]oxazolo[4,5-b]pyridin-5-yl]phenol

The apparatus was dried by heating with a heat gun under vacuum. The mixture of 5-chloro-2-(2,6-dichlorooxazolo[4,5-b]pyridin-5-yl)phenol (20.0 mg, 0.06 mmol, 1.0 eq) in DMF (0.5 mL) was added DIEA (16.35 mg, 0.13 mmol, 2.0 eq), and the mixture was stirred at 25° C. for 12 hours. The reaction mixture was quenched by water (5 mL), then extracted by EA (10 mL×3), washed with brine (20 mL×2), anhydrous sodium sulfate, filtrated and the filtrate was concentrated under reduce pressure. The crude was purified by prep-HPLC (Column: Waters Xbridge 150×25 mm×5 μm; condition: water (NH₄HCO₃)—CAN; Begin B: 41; End B: 71; Gradient Time(min): 9; 100% B Hold Time(min): 2; FlowRate (ml/min): 25) to afford the title compound (4.49 mg, 0.01 mmol, 17% yield) as a white solid. LCMS: m/z 407.1 [M+H]+, ESI pos.

### Example 128

### 3,5-Dimethyl-2-[2-[[(3R)-1-methyl-3-piperidyl]amino]oxazolo[4,5-b]pyridin-5-yl]phenol;2,2,2-trifluoroacetic acid

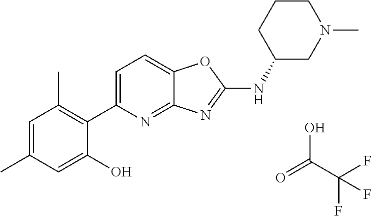

According to GP2a 3,5-dimethyl-2-(4,4,5,5-tetramethyl-1,3,2-dioxaborolan-2-yl)phenol (Example 123, step D) (63.79 mg, 0.26 mmol, 2.0 eq) and 5-bromo-N-[(3R)-1-methyl-3-piperidyl]oxazolo[4,5-b]pyridin-2-amine (40.0 mg, 0.13 mmol, 1.0 eq) were reacted. The crude was purified by column chromatography (C₁₈, 0.1% TFA in water/MeCN) to afford the title compound (6.83 mg, 11% yield) as yellow oil. LCMS: m/z 353.2. [M+H⁺, ESI pos.

### Example 129

### 3-Chloro-2-[2-[[(3R)-1-ethyl-3-piperidyl]amino]oxazolo[4,5-b]pyridin-5-yl]-5-fluoro-phenol;2,2,2-trifluoroacetic acid

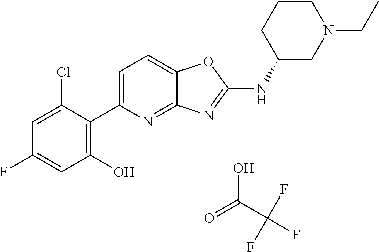 [SMILES: CCN1CCC[C@@H](Nc2nc3nc(-c4c(O)cc(F)cc4Cl)ccc3o2)C1 ; O=C(O)C(F)(F)F]

### Step A: 3-Chloro-5-fluoro-2-iodo-phenol and 5-chloro-3-fluoro-2-iodophenol

The apparatus was dried by heating with a heatgun under vacuum. To a mixture of 3-chloro-5-fluoro phenol (1.0 g, 6.82 mmol, 1.0 eq) in toluene (10 mL) was added NaH (0.55 g, 13.7 mmol, 2.0 eq, 60%) at 0° C. under N₂, stirred for 30 mins to get a yellow solution, then iodine (1.73 g, 6.82 mmol, 1.0 eq) was added and stirred for 15.5 hrs at 25° C. and a white mixture was obtained. The mixture was quenched by addition of water (20 mL) and extracted with EtOAc (30 mL×3), washed with brine (40 mL×2), dried over anhydrous Na₂SO₄, filtered and the filtrate was concentrated under reduce pressure. The crude was purified by column chromatography (silica gel, Petroleum ether/Ethyl acetate=10/1 to 5/1) to give a mixture of 3-chloro-5-fluoro-2-iodo-phenol and 5-chloro-3-fluoro-2-iodophenol=2:1 (700 mg, 38% yield) as a yellow oil. ¹H NMR (400 MHz, DMSO-d₆) δ [ppm]: 11.26 (br. s, 1H), 7.00 (d, 1H), 6.65 (d, 1H).

### Step B: 1-Chloro-5-fluoro-2-iodo-3-methoxy-benzene and 5-chloro-1-fluoro-2-iodo-3-methoxybenzene

The apparatus was dried by heating with a heatgun under vacuum. To a solution of 3-chloro-5-fluoro-2-iodo-phenol (700 mg, 2.57 mmol, 1.0 eq) in DMF (1 mL) was added Cs₂CO₃ (1674.2 mg, 5.14 mmol, 2.0 eq) and the mixture was stirred at 25° C. for 5 mins, then Mel (1094.6 mg, 7.71 mmol, 3.0 eq) was added to the mixture and stirring was continued for 2 hours. The reaction mixture was quenched by addition of water (10 mL) and extracted by EtOAc (20 mL×3), washed with brine (30 mL×2), dried over anhydrous Na₂SO₄, filtered and the filtrate was concentrated under reduce pressure. The crude was purified by column chromatography on silica gel (petroleum ether:ethyl acetate=20:0 to 10:1) to afford a mixture of 1-chloro-5-fluoro-2-iodo-3-methoxy-benzene and 5-chloro-1-fluoro-2-iodo-3-methoxybenzene=2:1 (450 mg, 61% yield) as a yellow solid. ¹H NMR (DMSO-d₆, 400 Hz) δ [ppm]: 7.20 (d, 1H), 6.96 (d, 1H), 3.87 (s, 3H).

### Step C: 2-(2-Chloro-4-fluoro-6-methoxy-phenyl)-4,4,5,5-tetramethyl-1,3,2-dioxaborolane and 2-(4-chloro-2-fluoro-6-methoxyphenyl)-4,4,5,5-tetramethyl-1,3,2-dioxaborolane

The apparatus was dried by heating with a heatgun under vacuum. To a mixture of 1-chloro-5-fluoro-2-iodo-3-methoxy-benzene (200 mg, 0.7 mmol, 1.0 eq) and 2-isopropoxy-4,4,5,5-tetramethyl-1,3,2-dioxaborolane (194.9 mg, 1.05 mmol, 1.5 eq) in THF (3 mL) was added n-BuLi (0.42 mL, 0.84 mmol, 1.2 eq) at −78° C., and the mixture was stirred at −10° C. for 30 mins to get a colorless solution. The reaction mixture was quenched by addition of aq. sat. NH₄Cl solution (5 mL), then diluted with water (5 mL). After extraction with EtOAc (30 mL×3), the organic phase was washed with brine (40 mL×2), dried over anhydrous Na₂SO₄, filtered and the filtrate was concentrated under reduce pressure. The crude was purified by column chromatography on silica gel (petroleum ether:ethyl acetate=1:0 to 5:1) to afford a mixture of 2-(2-chloro-4-fluoro-6-methoxy-phenyl)-4,4,5,5-tetramethyl-1,3,2-dioxaborolane and 2-(4-chloro-2-fluoro-6-methoxyphenyl)-4,4,5,5-tetramethyl-1,3,2-dioxaborolane=2:1 (70.0 mg, 35% yield) as a yellow oil. ¹H NMR (CD₃OD, 400 MHz) δ [ppm]: 6.74 (s, 1H), 6.71 (s, 1H), 3.76 (s, 3H), 1.30 (s, 12H).

### Step D: (2-Chloro-4-fluoro-6-hydroxy-phenyl)boronic acid and (4-chloro-2-fluoro-6-hydroxyphenyl)boronic acid

The apparatus was dried by heating with a heatgun under vacuum. To a mixture of 2-(2-chloro-4-fluoro-6-methoxy-phenyl)-4,4,5,5-tetramethyl-1,3,2-dioxaborolane (70.0 mg, 0.24 mmol, 1.0 eq) in DCM (0.5 mL) BBr₃ (612.03 mg, 2.44 mmol, 10.0 eq) was added under nitrogen atmosphere to get a white solution, and the mixture was stirred at −60° C. for 10 minutes, then stirred at 25° C. for 1 hour. The reaction was quenched by addition of ice water (2 mL) and the neutralized by addition of aq. ammonia solution to get a yellow solution. After removal of the solvent, the solution was purified by reversed phase flash (0.1% NH₃H₂O water/ACN condition) to yield a mixture of (2-chloro-4-fluoro-6-hydroxy-phenyl)boronic acid and (4-chloro-2-fluoro-6-hydroxyphenyl)boronic acid=2:1 (25.0 mg, 38% yield) as a white solid. LCMS: m/z 189.0 [M−H]⁻, ESI neg.

### Step E: (R)-3-Chloro-2-(2-((1-ethylpiperidin-3-yl)amino)oxazolo[4,5-b]pyridin-5-yl)-5-fluorophenol 2,2,2-trifluoroacetate and (R)-5-chloro-2-(2-((1-ethylpiperidin-3-yl)amino)oxazolo[4,5-b]pyridin-5-yl)-3-fluorophenol 2,2,2-trifluoroacetate

The apparatus was dried by heating with a heatgun under vacuum. To a mixture of 5-bromo-N-[(3R)-1-ethyl-3-piperidyl]oxazolo[4,5-b]pyridin-2-amine;2,2,2-trifluoroacetic acid (Example 21, step H) (32.3 mg, 0.07 mmol, 0.7 eq), (2-chloro-4-fluoro-6-hydroxy-phenyl)boronic acid and (4-chloro-2-fluoro-6-hydroxyphenyl)boronic acid (20.0 mg, 0.11 mmol, 1.0 eq 70%) and K₂CO₃ (29.0 mg, 0.21 mmol, 2.0 eq) in 1,4-dioxane (0.5 mL) and water (0.1 mL) was added Pd(dppf)Cl₂ (7.68 mg, 0.01 mmol, 0.1 eq) to get a red mixture, then the mixture was stirred at 90° C. for 2 hours under N2 to get a brown solution. The reaction mixture was quenched by addition of water (2 mL) and then diluted with MeOH (2 mL) to get a brown solution. The solution was purified by reversed phase flash (C₁₈ cartridge, 0.1% TFA water/ACN condition) to yield 3-chloro-2-[2-[[(3R)-1-ethyl-3-piperidyl]amino]oxazolo[4,5-b]pyridin-5-yl]-5-fluoro-phenol;2,2,2-trifluoroacetic acid (2.51 mg, 4.7% yield) as a white solid. LCMS: m/z 391.1 [M+H]⁺, ESI pos.

¹H NMR (CD₃OD, 400 MHz) δ 7.79 (d, 1H), 7.13 (d, 1H), 7.65 (d, 1H), 7.63 (d, 1H), 4.20-4.14 (m, 1H), 4.00-3.95 (m, 1H), 3.68-3.55 (m, 1H), 3.26-3.24 (m, 2H), 3.00-2.98 (m, 1H), 2.90-2.75 (m, 1H), 2.26-2.24 (m, 1H), 2.18-2.10 (m, 1H), 2.00-1.80 (m, 1H), 1.76-1.60 (m, 1H), 1.39-1.36 (m, 3H). 5-Chloro-2-[2-[[(3R)-1-ethyl-3-piperidyl]amino]oxazolo[4,5-b]pyridin-5-yl]-3-fluoro-phenol;2,2,2-trifluoroacetic acid (1.5 mg, 2.8% yield) was obtained as a yellow solid. LCMS: m/z 391.1 [M+H]⁺, ESI pos. ¹H NMR (400 MHz, DMSO-d₆) δ 12.6 (br. s, 0.45 H), 9.45 (br. s, 0.30 H), 9.03 (br. s, 0.38 H), 7.92 (d, 1H), 7.40 (d, 1H), 6.90 (d, 1H), 6.84 (s, 1H), 3.80-3.70 (m, 1H), 3.51-3.48 (m, 1H), 3.33-3.23 (m, 2H), 3.00-2.90 (m, 1H), 2.88-2.68 (m, 1H), 2.18-2.10 (m, 1H), 2.00-1.80 (m, 2H), 1.76-1.60 (m, 1H), 1.58-1.50 (m, 1H). 1.39-1.36 (m, 3H).

### Example 130

### 5-(Difluoromethoxy)-2-[2-[[(3R)-1-ethyl-3-piperidyl]amino]oxazolo[4,5-b]pyridin-5-yl]-3-methyl-phenol;2,2,2-trifluoroacetic acid

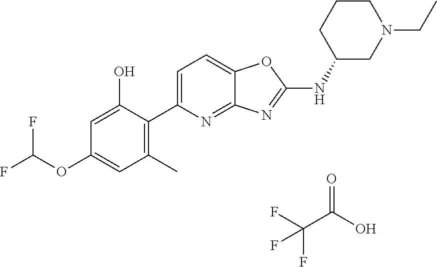 [SMILES: CCN1CCC[C@@H](Nc2nc3nc(-c4c(C)cc(OC(F)F)cc4O)ccc3o2)C1 ; O=C(O)C(F)(F)F]

### Step A: 3-(Difluoromethoxy)-5-methylphenol

To a solution of orcinol (CAS #504-15-4, 5.00 g, 40.3 mmol, 1.0 eq) in DMF (150 mL) and water (25 mL) was added Cs₂CO₃ (2.62 g, 80.6 mmol, 2.0 eq), followed by sodium 2-chloro-2,2-difluoro-acetate (CAS #1895-39-2, 7417.6 mg, 48.3 mmol, 1.2 eq), at 25° C. after stirred for 15 min, the reaction mixture was warmed to 100° C. and stirred for 4 hours under nitrogen atmosphere. Subsequently, the reaction mixture was quenched with 1N HCl (pH˜5), extracted with EtOAc (100 mL×3), washed with brine (20 mL×2), dried over Na₂SO₄, filtrated and the filtrate was concentrated under reduced pressure. The crude was purified by column chromatography on silica gel (petroleum ether:ethyl acetate=1:0 to 6:1) to afford the title compound (1.70 g, 24% yield) as yellow oil. ¹H NMR (CDCl₃, 400 MHz,) δ [ppm]: 6.66-6.29 (m, 4H), 4.79 (br s, 1H), 2.31 (s, 3H).

### Step B: 5-(Difluoromethoxy)-2-iodo-3-methylphenol

To a solution of 3-(difluoromethoxy)-5-methyl-phenol (390 mg, 2.24 mmol, 1.0 eq) in toluene (4 mL) was added NaH (179.2 mg, 4.48 mmol, 2.0 eq 60%). After stirring at 0° C. for 10 min, a solution of iodine (397.9 mg, 1.57 mmol, 0.7 eq) in toluene (4 mL) was added dropwise and the reaction mixture was stirred at 25° C. for 12 hrs under nitrogen atmosphere. Afterwards, the reaction mixture was quenched with 1N HCl (pH˜4), extracted with EtOAc (100 mL×3), washed with brine (20 mL×2), the organic layer was dried over Na₂SO₄, filtered and the filtrate was concentrated under reduced pressure. The crude was purified by prep-TLC (petroleum ether:ethyl acetate=3:1, Rf=0.5) to afford the title compound (250 mg, 37% yield) as a yellow oil. LCMS: m/z 298.8 [M−H]⁻, ESI neg.

### Step C: 1-(Benzyloxy)-5-(difluoromethoxy)-2-iodo-3-methylbenzene

To a solution of 5-(difluoromethoxy)-2-iodo-3-methyl-phenol (250.0 mg, 0.83 mmol, 1.0 eq) in N,N-dimethylformamide (5 mL) was added K₂CO₃ (230.3 mg, 1.67 mmol, 2.0 eq), followed by dropwise addition of benzyl bromide (0.15 mL, 1.25 mmol, 1.5 eq) and the reaction mixture was stirred at 25° C. for 2 hours under nitrogen atmosphere. Afterwards, the reaction mixture was quenched with a sat. aq. solution of NH₄Cl (30 mL), extracted with EtOAc (100 mL×3), the organic layer was dried over Na₂SO₄, filtered and the filtrate was concentrated under reduced pressure. The crude was purified by column chromatography on silica gel (petroleum ether:ethyl acetate=10:0 to 20:1) to afford the title compound (300 mg, 92% yield) as a colorless oil.

### Step D: 2-(2-(Benzyloxy)-4-(difluoromethoxy)-6-methylphenyl)-4,4,5,5-tetramethyl-1,3,2-dioxaborolane

To a solution of 1-benzyloxy-5-(difluoromethoxy)-2-iodo-3-methyl-benzene (230 mg, 0.59 mmol, 1.0 eq) and 2-isopropoxy-4,4,5,5-tetramethyl-1,3,2-dioxaborolane (219.4 mg, 1.18 mmol, 2.0 eq) in THF (5 mL) was added n-BuLi (0.46 mL, 1.15 mmol, 1.95 eq) dropwise at −60° C. and stirring was continued at −60° C. for 1 h under nitrogen atmosphere. The reaction was quenched with a sat. aq. solution of NH₄Cl (30 mL), extracted with EtOAc (100 mL×3), washed with brine (10 mL). The organic layer was dried over anhydrous Na₂SO₄, filtered and the filtrate was concentrated under reduced pressure. The crude was purified by column chromatography on silica gel (petroleum ether:ethyl acetate=1:0 to 10:1) to afford the title compound (240 mg, 0.62 mmol, 89% yield) as a yellow oil. ¹H NMR (CDCl₃, 400 MHz,) δ [ppm]: 7.46 (d, 1H), 7.38-7.32 (m, 3H), 6.64-6.27 (m, 3H), 5.00 (s, 2H), 2.37 (s, 3H), 1.37 (s, 12H).

### Step E: 5-(Difluoromethoxy)-3-methyl-2-(4,4,5,5-tetramethyl-1,3,2-dioxaborolan-2-yl)phenol

A solution of 2-[2-benzyloxy-4-(difluoromethoxy)-6-methyl-phenyl]-4,4,5,5-tetramethyl-1,3,2-dioxaborolane (160.0 mg, 0.41 mmol, 1.0 eq) in EtOAc (6 mL) and methanol (2 mL.) was degassed and purged with nitrogen three times, then Pd/C (60 mg) was added and the mixture was degassed and purged with hydrogen three times. The reaction mixture was stirred at 20° C. for 2 hrs under hydrogen atmosphere (15 psi). Afterwards, the reaction mixture was filtered, the solid was washed with MeOH (20 mL×2), and the filtrate was concentrated under reduced pressure. The crude was purified by column chromatography on silica gel (petroleum ether:ethyl acetate=10:1 to 0:1) to afford the title compound (120.0 mg, 90% yield) as a yellow oil. LCMS: m/z 301.1 [M+H]⁺, ESI pos.

### Step F: 5-(Difluoromethoxy)-2-[2-[(3R)-1-ethyl-3-piperidyl]amino]oxazolo[4,5-b]pyridin-5-yl]-3-methyl-phenol;2,2,2-trifluoroacetic acid

A mixture of 5-bromo-N-[(3R)-1-ethyl-3-piperidyl]oxazolo[4,5-b]pyridin-2-amine;2,2,2-trifluoroacetic acid (Example 21, step H) (30.0 mg, 0.07 mmol, 1.0 eq), 5-(difluoromethoxy)-3-methyl-2-(4,4,5,5-tetramethyl-1,3,2-dioxaborolan-2-yl)phenol (30.1 mg, 0.1 mmol, 1.47 eq) and K₂CO₃ (47.14 mg, 0.34 mmol, 5.0 eq) in 1,4-dioxane (3 mL) and water (0.3 mL) was degassed and purged with nitrogen atmosphere three times. Then Pd(dppf)Cl₂ (4.99 mg, 0.01 mmol, 0.1 eq) was added and the reaction mixture was degassed and purged with nitrogen three times. Afterwards, the mixture was stirred at 100° C. for 2 hrs. After cooling to r.t., the mixture was diluted with MeOH (20 mL) and EtOAc (20 mL), filtered and the filtrate was concentrated under reduced pressure. The crude was purified by reversed-phase flash (0.1% TFA/MeCN condition) to afford the title compound (19.3 mg, 53% yield) as a yellow solid. LCMS: m/z 419.2 [M+H]⁺, ESI pos.

### Example 131

### 2-[2-[(1-Ethylpyrrolidin-3-yl)amino]oxazolo[4,5-b]pyridin-5-yl]-3-methyl-5-(trifluoromethyl)phenol

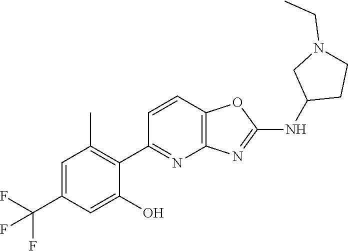 [SMILES: CCN1CCC(Nc2nc3nc(-c4c(C)cc(C(F)(F)F)cc4O)ccc3o2)C1]

### Step A: 3-[(5-Chlorooxazolo[4,5-b]pyridin-2-yl)amino]pyrrolidine-1-carboxylic acid tert-butyl ester

Following to GP1, 5-chloro-2-(methylthio)oxazolo[4,5-b]pyridine (150 mg, 0.748 mmol, 1.0 eq) and 3-aminopyrrolidine-1-carboxylic acid tert-butyl ester (155 mg, 0.83 mmol, 1.1 eq) were reacted to afford the title compound (265 mg, 94%) as a brown solid. LCMS m/z: 383.1 [M+H−tBu]⁺, ESI pos.

Step B: (5-Chlorooxazolo[4,5-b]pyridin-2-yl)-pyrrolidin-3-yl-amine

3-[(5-Chlorooxazolo[4,5-b]pyridin-2-yl)amino]pyrrolidine-1-carboxylic acid tert-butyl ester (262 mg, 0.7 mmol, 1.0 eq) was dissolved in MeOH (2.5 mL) and 4 M HCl in dioxane (1.57 mL, 6.26 mmol, 9.0 eq) was added dropwise to the reaction mixture and stirred at r.t. for 2 hrs. The solvent was removed and the crude taken up in ethyl acetate (25 mL) and water (10 mL). The water phase was extracted back twice with EtOAc (20 mL). LC-MS showed that product was in the water layer. To the water layer 1N NaOH (5 mL) was added (pH 10). A light brown solid precipitated. The water phase was extracted again DCM (30 mL). The organic phases were combined and washed with brine (10 mL). The combined organic layers were dried over anhydrous sodium sulfate, filtered and concentrated in vacuo. The crude product was purified by flash chromatography (silica gel, DCM/MeOH with 1% NH₃ 0-10% MeOH) to afford the title compound (54 mg, 27% yield) as a brown solid. LCMS m/z: 239.1 [M+H]⁺, ESI pos.

### Step C: (5-Chlorooxazolo[4,5-b]pyridin-2-yl)-(1-ethylpyrrolidin-3-yl)amine

Following to GP4 (5-chlorooxazolo[4,5-b]pyridin-2-yl)-pyrrolidin-3-yl-amine;hydrochloride (50 mg, 0.15 mmol, 1.0 eq) and acetaldehyde (13.4 mg, 15.19 μL, 0.3 mmol, 2.0 eq) to afford the title compound (11 mg, 28% yield) as light yellow solid. LCMS m/z: 267.1 [M+H]⁺, ESI pos.

### Step D: 2-[2-[(1-Ethylpyrrolidin-3-yl)amino]oxazolo[4,5-b]pyridin-5-yl]-3-methyl-5-(trifluoromethyl)phenol

Following to GP2b (5-chlorooxazolo[4,5-b]pyridin-2-yl)-(1-ethylpyrrolidin-3-yl)amine (10 mg, 0.037 mmol, 1.0 eq) and [2-hydroxy-6-methyl-4-(trifluoromethyl)phenyl]boronic acid (12 mg, 0.054 mmol, 1.45 eq) were reacted to afford the title compound (8.5 mg, 50%) as yellow solid. LCMS m/z: 407.2 [M+H]⁺, ESI pos.

### Example 132

### 3,5-Dichloro-2-[2-[[(3R)-1-ethyl-3-piperidyl]amino]oxazolo[4,5-b]pyridin-5-yl]phenol;2,2,2-trifluoroacetic acid

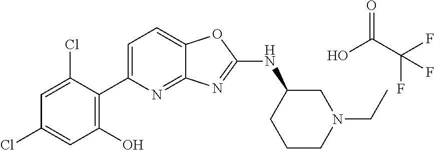

### Step A: 3,5-Dichloro-2-iodophenol

A 100 mL-reactor was set under inert atmosphere. Iodine (3.11 g, 12.3 mmol, 1.0 eq) was dissolved in toluene (10 mL) and stirred until full dissolution (0.5 h) at 25° C., toluene (5 mL) was fed into the reactor followed by addition of NaH (982 mg, 24.5 mmol, 2.0 eq 60%), The suspension was cooled to 0-5° C. and a solution of 3,5-dichlorophenol (2.0 g, 12.27 mmol, 1.0 eq) in toluene (8 mL) was added during 0.5 h keeping the internal temperature <10° C. After complete addition, stirring was continued for 4 hrs. Afterwards, the reaction was quenched by addition of aq. 1N HCl (10 mL) keeping the internal temperature <15° C. The layers were separated and the aqueous phase was extracted with EtOAc (100 mL). The combined organic phases were washed with brine, dried over Na₂SO₄, filtered and the filtrate was concentrated under vacuum. The residue was purified by silica gel column chromatography (petroleum ether:ethyl acetate=100:1 to 10/1) to give the title compound (1.4 g, 39% yield) as a yellow solid. LCMS: m/z 286.7 [M−H]⁻, ESI neg.

### Step B: 1,5-Dichloro-2-iodo-3-methoxybenzene

A 100 mL-reactor was set under inert atmosphere. To a mixture of 3,5-dichloro-2-iodo-phenol (1.4 g, 4.85 mmol, 1.0 eq) and Cs₂CO₃ (2.37 g, 7.27 mmol, 1.5 eq) in DMF (10 mL) was added dropwise Mel (1375.7 mg, 9.69 mmol, 2.0 eq), under nitrogen atmosphere at 0° C., then the mixture was stirred at 50° C. for 4 hrs. The reaction was subsequently quenched by addition of 1N HCl (10 mL) keeping the internal temperature <15° C. The layers were separated and the aqueous phase was extracted with EtOAc (100 mL). The combined organic phases were washed with brine, dried over Na₂SO₄ and filtered. The filtrate was concentrated under vacuum. The residue was purified by silica gel column chromatography (petroleum ether:ethyl acetate=100:1 to 10/1) to give the title compound (1.0 g, 68% yield) as a white solid. ¹H NMR (CDCl₃, 400 MHz) δ [ppm]: 7.14 (s, 1H), 6.69 (s, 1H), 3.90 (s, 3H).

### Step C: 2-(2,4-Dichloro-6-methoxyphenyl)-4,4,5,5-tetramethyl-1,3,2-dioxaborolane

To a mixture of 1,5-dichloro-2-iodo-3-methoxy-benzene (500.0 mg, 1.65 mmol, 1.0 eq), 2-isopropoxy-4,4,5,5-tetramethyl-1,3,2-dioxaborolane (552.8 mg, 2.97 mmol, 1.8 eq), in dry THF (10 mL) was added dropwise nBuLi (1.19 mL, 2.97 mmol, 1.8 eq) under nitrogen atmosphere and stirred for −70° C. at 30 mins, The mixture was poured into aq. sat. NH₄Cl solution (20 mL) and extracted with ethyl acetate (100 mL×3). The combined organic layers were washed with brine (20 mL), dried over anhydrous sodium sulfate, filtered, and the filtrate was concentrated under reduced pressure. The residue was purified by silica gel column chromatography (petroleum ether:ethyl acetate=100:1 to 20/1) to give the title compound (270.0 mg, 54% yield) as a white solid. ¹H NMR (CD₃OD, 400 MHz) δ [ppm]: 6.98 (s, 1H), 6.92 (s, 1H), 3.79 (s, 3H), 1.36 (s, 12H).

### Step D: (2,4-Dichloro-6-hydroxyphenyl)boronic acid

To a solution of 2-(2,4-dichloro-6-methoxy-phenyl)-4,4,5,5-tetramethyl-1,3,2-dioxaborolane (270 mg, 0.89 mmol, 1.0 eq) in DCM (2 mL) was dropwise added BBr₃ (2.30 g, 17.8 mmol, 20.0 eq) under nitrogen atmosphere at −60° C. and then the mixture was stirred at 25° C. for 2 hrs. The reaction mixture was poured into 10 mL water at 0° C., the pH was adjusted to 8 by addition of aq. 1N NaOH solution. The mixture was washed with DCM (10 mL×3) and subsequently was acidified with aq. 1N HCl solution to pH=4 and a white precipitate was formed. It was collected by filtration and air-dried to give the title compound (0.8 g, 87% yield) as a white solid LCMS: m/z 203.9, [M−H]⁻, ESI neg.

### Step E: 3,5-Dichloro-2-[2-[[(3R)-1-ethyl-3-piperidyl]amino]oxazolo[4,5-b]pyridin-5-yl]phenol;2,2,2-trifluoroacetic acid

The apparatus was dried by heating with a heat gun under vacuum. To a solution of (2,4-dichloro-6-hydroxyphenyl)boronic acid (353.15 mg, 0.340 mmol, 3 eq), 5-bromo-N-[(3R)-1-ethyl-3-piperidyl]oxazolo[4,5-b]pyridin-2-amine;2,2,2-trifluoroacetic acid (Example 21, step H) (50.0 mg, 0.110 mmol, 1 eq) and K₂CO₃ (78.55 mg, 0.570 mmol, 5 eq) in 1,4-dioxane (2 mL)/water (0.2 mL) was added Pd(dppf)Cl₂ (8.32 mg, 0.010 mmol, 0.1 eq) under nitrogen at 25° C. The reaction mixture was stirred at 90° C. for 3 hrs. After cooling to r.t. EtOAc (40 mL) and water (40 mL) were added and layers were separated. The aqueous phase was extracted with EtOAc (50 mL×5). Combined extracts were washed with brine (60 mL), dried over MgSO₄, filtered, and concentrated under vacuum. The crude was purified by column chromatography (C₁₈, 0.1% TFA in water/MeCN) to afford the title compound (7.95 mg, 17% yield) as a yellow solid. LCMS: m/z 407.1, [M+H]⁺, ESI pos.

### Example 133

### 2-[2-[[(3R)-1-Ethyl-3-piperidyl]amino]-6-fluoro-1H-imidazo[4,5-b]pyridin-5-yl]-5-(trifluoromethyl)phenol

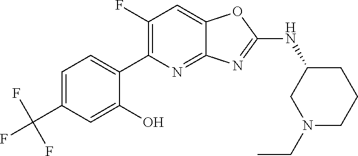 [SMILES: CCN1CCC[C@@H](Nc2nc3nc(-c4ccc(C(F)(F)F)cc4O)c(F)cc3o2)C1]

### Step A: 2,6-Dichloro-3-fluoro-5-nitropyridine

To a solution of H₂SO₄ (21.3 g, 216.9 mmol, 4.0 eq) was added 2,6-dichloro-3-fluoro-pyridine (CAS #52208-50-1, 9.0 g, 54.2 mmol, 1.0 eq) at 0° C. to get a light yellow solution. Afterwards, fuming nitric acid (26.3 g, 271.1 mmol, 5.0 eq) was added to the mixture at 0° C. to get a yellow solution, and the mixture was stirred at 0° C. for 1 hour, then the mixture was stirred at 100° C. for 12 hrs. The reaction mixture was quenched by 100 mL of ice water, then extracted with EtOAc (200 mL×3), washed with brine (200 mL×2), filtered and the filtrate was concentrated under reduce pressure to get a yellow solid. The crude was purified by column chromatography on silica gel (petroleum ether:ethyl acetate=1:0 to 10:1) to obtain the title compound (5.40 g, 47% yield) as a yellow solid. ¹H NMR (DMSO-δ₆, 400 MHz) δ [ppm]: 8.97 (d, 1H).

### Step B: 2-Chloro-5-fluoro-6-(2-methoxy-4-(trifluoromethyl)phenyl)-3-nitropyridine

To a solution of 2,6-dichloro-3-fluoro-5-nitro-pyridine (450 mg, 2.13 mmol, 1.0 eq), 2-methoxy-4-(trifluoromethyl)-phenylboronic acid (516.1 mg, 2.35 mmol, 1.1 eq; CAS: 312936-89-3), CsF (991.8 mg, 6.4 mmol, 3.0 eq) in 1,4-dioxane (5 mL)/water (1 mL) under N₂ at 25° C. The reaction mixture was stirred at 100° C. for 3 hrs. After cooling to r.t., EtOAc (40 mL) and water (40 mL) were added and layers were separated. The aqueous phase was extracted with EtOAc (50 mL×5). Combined extracts were washed with brine (60 mL), dried over Na₂SO₄, filtered, and concentrated under vacuum. The crude was purified by reversed phase flash (water (0.1% TFA)-MeCN) to afford the title compound (300 mg, 28% yield) as a yellow solid. ¹H NMR (CD₃OD, 400 MHz) δ [ppm]: 8.50-8.47 (m, 1H), 7.66-7.47 (m, 1H), 7.44-7.29 (m, 2H), 3.92 (s, 3H).

### Step C: 5-Fluoro-6-(2-methoxy-4-(trifluoromethyl)phenyl)-3-nitropyridin-2-amine

A solution of 2-chloro-5-fluoro-6-[2-methoxy-4-(trifluoromethyl)phenyl]-3-nitro-pyridine (250.0 mg, 0.71 mmol, 1.0 eq) and NH₃/MeOH (2.04 mL, 14.26 mmol, 20.0 eq) was stirred at 75° C. for 12 hrs. The reaction mixture was concentrated under vacuum to give a residue which was purified by column chromatography on silica gel (petroleum ether:ethyl acetate=1:0 to 10:1) to afford the title compound (150.0 mg, 0.45 mmol, 58% yield) as a yellow solid. LCMS: m/z 331.8, [M+H]⁺, ESI pos.

### Step D: 5-Fluoro-6-(2-methoxy-4-(trifluoromethyl)phenyl)pyridine-2,3-diamine

To a solution of 5-fluoro-6-[2-methoxy-4-(trifluoromethyl)phenyl]-3-nitro-pyridin-2-amine (770 mg, 2.32 mmol, 1.0 eq) in methanol (10 mL) was added Pd/C (50.0 mg, 10%) under N₂. The suspension was degassed under vacuum and purged with H₂ several times. The mixture was stirred under H₂ balloon for 2 hrs at 20° C. The reaction mixture was filtered and the filter cake was washed with MeOH (10 mL×5). The combined filtrates were concentrated to dryness to give the title compound (600.0 mg, 65% yield) as a yellow solid. LCMS: m/z 302.2, [M+H]⁺, ESI pos.

### Step E: 6-Fluoro-5-(2-methoxy-4-(trifluoromethyl)phenyl)-3H-imidazo[4,5-b]pyridin-2-ol

To a solution of 5-fluoro-6-[2-methoxy-4-(trifluoromethyl)phenyl]pyridine-2,3-diamine (600.0 mg, 1.99 mmol, 1.0 eq) in THF (20 mL) was added CDI (580.8 mg, 3.59 mmol, 1.8 eq), and the mixture was stirred at 25° C. for 12 hrs. The reaction mixture was concentrated under vacuum. The crude was purified by column chromatography on silica gel (petroleum ether:ethyl acetate=1:0 to 2:1) to obtain the title compound (500 mg, 75% yield) as a white solid. LCMS: m/z 328.1, [M+H]⁺, ESI pos.

### Step F: 2-Chloro-6-fluoro-5-(2-methoxy-4-(trifluoromethyl)phenyl)-3H-imidazo[4,5-b]pyridine

A mixture of 6-fluoro-5-[2-methoxy-4-(trifluoromethyl)phenyl]-3H-imidazo[4,5-b]pyridin-2-ol (500 mg, 1.53 mmol, 1.0 eq) and POCl₃ (9.37 g, 61.12 mmol, 40.0 eq) was stirred at 110° C. for 2 hrs. The reaction mixture was concentrated under vacuum to give a residue which was purified by column chromatography (silica gel, petroleum ether:ethyl acetate=1:0 to 2:1) to obtain the title compound (150 mg, 27% yield) as a white solid. LCMS: m/z 346.1 [M+H]⁺, ESI pos.

### Step G: N-[(3R)-1-Ethyl-3-piperidyl]-6-fluoro-5-[2-methoxy-4-(trifluoromethyl)phenyl]-3H-imidazo[4,5-b]pyridin-2-amine;2,2,2-trifluoroacetic acid

A solution of 2-chloro-6-fluoro-5-[2-methoxy-4-(trifluoromethyl)phenyl]-3H-imidazo[4,5-b]pyridine (150.0 mg, 0.43 mmol, 1.0 eq) and (3R)-1-ethylpiperidin-3-amine (556.38 mg, 4.34 mmol, 10.0 eq), DIEA (111.95 mg, 0.87 mmol, 2.0 eq) in NMP (3 mL) was stirred at 130° C. for 32 hrs. After cooling to r.t., the reaction mixture was directly concentrated under reduced pressure to give a residue which was purified on automated flash chromatography system (ACN-0.1% TFA) to yield the title compound (85.0 mg, 35% yield) as a yellow solid. LCMS: m/z 438.2 [M+H]+, ESI pos.

### Step H: 2-[2-[[(3R)-1-Ethyl-3-piperidyl]aminol-6-fluoro-1H-imidazo[4,5-b]pyridin-5-yl]-5-(trifluoromethyl)phenol

To a solution of N-[(3R)-1-ethyl-3-piperidyl]-6-fluoro-5-[2-methoxy-4-(trifluoromethyl)-phenyl]-3H-imidazo[4,5-b]pyridin-2-amine;2,2,2-trifluoroacetic acid (85.0 mg, 0.15 mmol, 1.0 eq) in DCM (1 mL) was dropwise added BBr₃ (0.4 g, 3.08 mmol, 20 eq) under N₂ at −60° C., and then the mixture was stirred at 25° C. for 2 hrs. The reaction mixture was quenched with MeOH (20 mL) at −60° C. Afterwards, the mixture was quenched with aq. ammonia solution (to pH˜8). The precipitate was filtered off and the filtrate was concentrated in vacuum to give the residue which was purified by reversed phase flash (ACN-0.1% NH₃H₂O) to obtain the title compound (15.5 mg, 23% yield) as a white solid. LCMS: m/z 424.2, [M+H]⁺, ESI pos.

### Example 134

### (3S,5R)-5-[[5-(4-Chloro-2-hydroxy-6-methyl-phenyl)oxazolo[4,5-b]pyridin-2-yl]amino]-1-ethyl-piperidin-3-ol

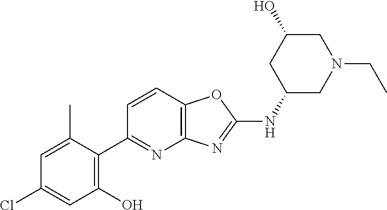 [SMILES: CCN1C[C@@H](O)C[C@@H](Nc2nc3nc(-c4c(C)cc(Cl)cc4O)ccc3o2)C1]

The title compound was obtained as an off-white amorph freeze-dried solid, using chemistry similar to that described in Example 28 (step 4) starting from (3S,5R)-5-[(5-chlorooxazolo[4,5-b]pyridin-2-yl)amino]-1-ethyl-piperidin-3-ol (Example 28, step 3) and (4-chloro-2-hydroxy-6-methyl-phenyl)boronic acid (CAS #1207961-50-9). LCMS: m/z 401.1; 403.1 (Cl isotopes) [M−H]−, ESI neg.

### Example 135

### 3-Methyl-2-[2-[4-(methylamino)tetrahydropyran-4-yl]oxazolo[4,5-b]pyridin-5-yl]-5-(trifluoromethyl)phenol;2,2,2-trifluoroacetic acid

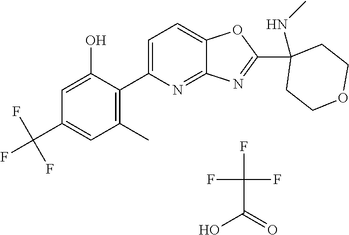 [SMILES: CNC1(c2nc3nc(-c4c(C)cc(C(F)(F)F)cc4O)ccc3o2)CCOCC1 ; O=C(O)C(F)(F)F]

### Step A: tert-Butyl (4-((6-bromo-3-hydroxypyridin-2-yl)carbamoyl)tetrahydro-2H-pyran-4-yl)carbamate

To a solution of DIEA (1.05 g, 8.15 mmol, 2.0 eq), 4-((tert-butoxycarbonyl)amino)tetrahydro-2H-pyran-4-carboxylic acid (1.00 g, 4.08 mmol, 1.0 eq), HOBt (825.6 mg, 6.12 mmol, 1.5 eq) and EDCI (1.22 g, 6.12 mmol, 1.5 eq) in DMF (10 mL) was added 2-amino-6-bromopyridin-3-ol (770.6 mg, 4.08 mmol, 1.0 eq). The mixture was stirred at 25° C. for 12 hrs. The mixture was quenched with ice water (20 mL) and extracted with ethyl acetate (30 mL×3). The combined organic phases were washed with brine (40 mL×3), dried over anhydrous sodium sulfate, filtered, and the filtrate was concentrated under reduced pressure. The residue was purified by silica gel column chromatography (petroleum ether:ethyl acetate=5:1 to 2:1) to afford the title compound (800 mg, 47% yield) as a pink solid. LCMS: m/z 418.0, [M+2+H]⁺ (Br isotope), ESI pos.

### Step B: tert-Butyl (4-(5-bromooxazolo[4,5-b]pyridin-2-yl)tetrahydro-2H-pyran-4-yl)carbamate

To a solution of DIAD (194.3 mg, 0.96 mmol, 2.0 eq) and PPh₃ (252 mg, 0.96 mmol, 2.0 eq) in THF (3 mL) was added tert-butyl (4-((6-bromo-3-hydroxypyridin-2-yl)carbamoyl)tetrahydro-2H-pyran-4-yl)carbamate (200.0 mg, 0.48 mmol, 1.0 eq), and the reaction mixture was stirred at 90° C. for 12 hrs. After cooling to ambient temperature, the reaction mixture was quenched by addition of water (10 mL), extracted with ethyl acetate (20 mL×3), washed with brine (30 mL×2), dried over anhydrous sodium sulfate, filtered and the filtrate was concentrated under reduce pressure. The residue was purified by column chromatography (C₁₈, 0.1% TFA in water-MeCN) to afford the title compound (60.0 mg, 31% yield) as a pink solid. LCMS: m/z 400.0, [M+2+H]⁺ (Br isotope), ESI pos.

### Step C: tert-Butyl (4-(5-bromooxazolo[4,5-b]pyridin-2-yl)tetrahydro-2H-pyran-4-yl)(methyl)carbamate

The apparatus was dried by heating with a heat gun under vacuum. To a mixture of tert-butyl (4-(5-bromooxazolo[4,5-b]pyridin-2-yl)tetrahydro-2H-pyran-4-yl)carbamate (150.0 mg, 0.38 mmol, 1.0 eq) in THF (3 mL) was added t-BuOK/THF (0.56 mL, 0.56 mmol, 1.5 eq) followed by addition of Mel (53.1 mg, 0.38 mmol, 1.0 eq). The mixture was stirred at 25° C. for 12 hrs and then concentrated under reduced pressure to afford the title compound (100.0 mg, 0.24 mmol, 64% yield) as a yellow oil. LCMS: m/z 414.0, [M+2+H]⁺ (Br isotope), ESI pos.

### Step D: 4-(5-Bromooxazolo[4,5-b]pyridin-2-yl)-N-methyltetrahydro-2H-pyran-4-amine

The apparatus was dried by heating with a heatgun under vacuum. To a mixture of tert-butyl (4-(5-bromooxazolo[4,5-b]pyridin-2-yl)tetrahydro-2H-pyran-4-yl)(methyl)carbamate (80.0 mg, 0.19 mmol, 1.0 eq) in DCM (1 mL) was added TFA (44.24 mg, 0.39 mmol, 2.0 eq) at 0° C. The mixture was stirred at 25° C. for 2 hrs then concentrated under reduced pressure to afford the title compound (80.0 mg, 97% yield) as yellow solid. LCMS: m/z 312.0 [M+H]⁺, ESI pos.

### Step E: 3-Methyl-2-[2-[4-(methylamino)tetrahydropyran-4-yl]oxazolo[4,5-b]pyridin-5-yl]-5-(trifluoromethyl)phenol;2,2,2-trifluoroacetic acid

The apparatus was dried by heating with a heatgun under vacuum. To a solution of 4-(5-bromooxazolo[4,5-b]pyridin-2-yl)-N-methyltetrahydro-2H-pyran-4-amine;2,2,2 trifluoroacetic acid (70.0 mg, 0.16 mmol, 1.0 eq), [2-hydroxy-6-methyl-4-(trifluoromethyl)phenyl]boronic acid (54.19 mg, 0.25 mmol, 1.5 eq) in 1,4-dioxane (2 mL) and water (0.4 mL) was added K₂CO₃ (68.0 mg, 0.49 mmol, 3.0 eq), then Pd(dppf)Cl₂ (18.0 mg, 0.02 mmol, 0.15 eq) was added to the mixture at 25° C. The mixture was stirred at 90° C. for 2 hrs under nitrogen atmosphere. After cooling to ambient temperature, the reaction mixture was diluted with MeOH (5 mL) and purified by C18 column chromatography (0.1% TFA water-MeCN) and re-purified by prep-HPLC (column: Phenomenex luna C₁₈ 150×25 mm×10 μm; condition: water (TFA)-CAN; Begin B: 25; End B: 55; Gradient Time(min): 11; 100% B Hold Time(min): 2; FlowRate (ml/min): 25) to afford the title compound (4.86 mg, 6% yield) as a white solid. LCMS: m/z 408.1 [M+H]⁺, ESI pos.

### Example 136

### 5-Chloro-2-[2-[[(3R)-1-ethyl-3-piperidyl]amino]-3H-imidazo[4,5-b]pyridin-5-yl]-3-methyl-phenol

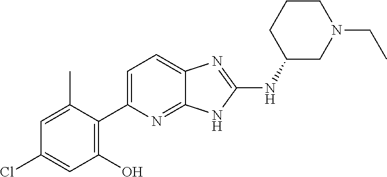 [SMILES: CCN1CCC[C@@H](Nc2nc3ccc(-c4c(C)cc(Cl)cc4O)nc3[nH]2)C1]

### Step A: (5-Bromo-1H-imidazo[4,5-b]pyridin-2-yl)-[(3R)-1-ethyl-3-piperidyl]amine

To a mixture of [(3R)-1-ethyl-3-piperidyl]amine;dihydrochloride (259.6 mg, 1.29 mmol, 3.0 eq) in N-methyl-2-pyrrolidinone (1 mL) was added N,N-diisopropyl ethylamine (500.36 mg, 676 μL, 3.87 mmol, 9.0 eq). After stirring for 10 min at ambient temperature 5-bromo-2-chloro-1H-imidazo[4,5-b]pyridine (CAS #1260669-88-2, 100 mg, 0.430 mmol, 1.0 eq) was added. The reaction mixture was stirred at 120° C. in a oilbath for 16 hrs. Afterwards, more [(3R)-1-ethyl-3-piperidyl]amine;dihydrochloride (259.6 mg, 1.29 mmol, 3.0 eq) was added and stirred continued at 150° C. for 24 hrs. The reaction mixture was quenched with water (1 mL) and extracted with TBME (2×30 mL). The organic layers were washed with water (30 mL) and brine (30 mL). The combined organic extracts were dried over sodium sulfate, filtered off and concentrated in vacuo. The residue was purified by flash chromatography (silica gel, 0-80% DCM/MeOH/NH₄OH 110:10:1 in DCM) to afford the title compound (96 mg, 63%) as light brown foam. LCMS: m/z 324.1; 326.1 (Br isotopes) [M+H]⁺, ESI pos.

### Step B: 5-Chloro-2-[2-[[(3R)-1-ethyl-3-piperidyl]amino]-3H-imidazo[4,5-b]pyridin-5-yl]-3-methyl-phenol

The title compound was obtained as a light brown foam (48 mg, 40%), using chemistry similar to that described in Example 18 (step 3) starting from (5-bromo-1H-imidazo[4,5-b]pyridin-2-yl)-[(3R)-1-ethyl-3-piperidyl]amine and (4-chloro-2-hydroxy-6-methyl-phenyl)boronic acid (CAS #1207961-50-9). LCMS: m/z 386.2; 388.3 (Cl isotopes) [M+H]⁺, ESI pos.

### Example 137

### 2-[2-[[(3R)-1-Ethyl-3-piperidyl]amino]oxazolo[4,5-b]pyridin-5-yl]-3-methyl-5-(trifluoromethoxy)phenol;2,2,2-trifluoroacetic acid

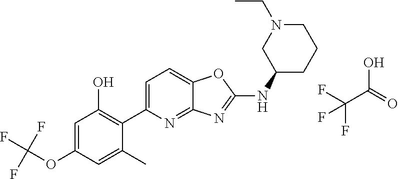

### Step A: 3-Methyl-5-(trifluoromethoxy) aniline

A mixture of 3-bromo-5-(trifluoromethoxy) aniline (6.8 g, 26.56 mmol, 1.0 eq), trimethylboroxine (11.38 mL, 39.84 mmol, 1.5 eq) and K₂CO₃ (7.33 g, 53.12 mmol, 2.0 eq) in 1,4-dioxane (400 mL) and water (80 mL) was degassed and purged with nitrogen three times and Pd(dppf)Cl₂ (0.97 g, 1.33 mmol, 0.05 eq) was added to the mixture. The mixture was stirred at 100° C. for 12 hrs. Afterwards, the mixture was poured into water (200 mL) and extracted with EtOAc (100 mL×3). The combined organic layers were washed with brine (200 mL), dried over anhydrous sodium sulfate, filtered and the filtrate was concentrated under reduced pressure and the crude was purified by silica gel column chromatography (petroleum ether:ethyl acetate=100:1 to 5/1) to afford the title compound (5.0 g, 98% yield) as yellow oil. ¹H NMR (CDCl₃, 400 MHz) δ [ppm]: 6.42 (s, 1H), 6.41 (s, 1H), 6.34 (s, 1H), 2.26 (s, 3H).

### Step B: 3-Methyl-5-(trifluoromethoxy)phenol

To a mixture of 3-methyl-5-(trifluoromethoxy) aniline (5.0 g, 26.2 mmol, 1.0 eq) in H₂SO₄ (10 mL)/water (10 mL) was added NaNO₂ (3.61 g, 52.3 mmol, 2.0 eq) and purged with nitrogen three times. The mixture was stirred at 80° C. for 3 hrs. Afterwards, the mixture was poured into water (200 mL) and extracted with EtOAc (100 mL×3). The combined organic layers were washed with brine (200 mL), dried over anhydrous sodium sulfate, filtered and the filtrate was concentrated under reduced pressure and purified by silica gel column chromatography (petroleum ether:ethyl acetate=100:1 to 5/1) to give the title compound (1.4 g, 23% yield) as a yellow oil. ¹H NMR (CDCl₃, 400 MHz) δ [ppm]: 6.62 (s, 1H), 6.58 (s, 1H), 6.53 (s, 1H), 2.32 (s, 3H)._LCMS: m/z 191.0, [M−H]⁻, ESI neg.

### Step C: 2,6-Diiodo-3-methyl-5-(trifluoromethoxy)phenol

To a mixture of 3-methyl-5-(trifluoromethoxy)phenol (1.40 g, 7.29 mmol, 1.00 eq) in toluene (20 mL) was added NaH (583 mg, 14.6 mmol, 2.0 eq, 60%) at 25° C. under nitrogen, stirred for 30 mins, then iodine (1849.4 mg, 7.29 mmol, 1.0 eq) was added and stirred for 15.5 hrs at 25° C. The mixture was poured into aq. 1N HCl (50 mL) and extracted with ethyl acetate (100 mL×3). The combined organic layers were washed with brine (20 mL), dried over anhydrous sodium sulfate, filtered, and the filtrate was concentrated under reduced pressure. The residue was purified by column chromatography (silica gel, petroleum ether:ethyl acetate=100:1 to 10/1) to give the title compound (2.0 g, 62% yield) as colorless oil. LCMS: m/z 442.8, [M−H]⁻, ESI neg.

### Step D: 2-Iodo-3-methyl-5-(trifluoromethoxy)phenol

To a solution of benzyl N-[(3R)-1-ethyl-3-piperidyl]carbamate (10.0 g, 38.12 mmol, 1 eq) in methanol (100 mL) was added Pd/C (100 mg, 10%) under nitrogen atmosphere. The suspension was degassed under vacuum and purged with H₂ several times. The mixture was stirred under H₂ balloon at 20° C. for 2 hrs. The suspension was filtered and the filter cake was washed with MeOH (100 mL×5). The combined filtrates were concentrated to dryness to give the title compound (1400.0 mg, 31% yield) as a white solid. LCMS: m/z 316.9, [M−H]⁻, ESI neg.

### Step E: 1-(Benzyloxy)-2-iodo-3-methyl-5-(trifluoromethoxy)benzene

To a solution of 2-iodo-3-methyl-5-(trifluoromethoxy)phenol (1.4 g, 1.36 mmol, 1.0 eq), K₂CO₃ (376.6 mg, 2.73 mmol, 2.0 eq) in DMF (20 mL) was added BnBr (350.11 mg, 2.05 mmol, 1.5 eq) under nitrogen. Then the mixture was stirred at 25° C. for 2 hrs. The reaction was quenched by addition of 1N HCl (10 mL) keeping the internal temperature <15° C. The layers were separated and the aqueous phase was extracted with EtOAc (100 mL). The combined organic phases were washed with brine, dried over Na₂SO₄ and filtered. The filtrate was concentrated under vacuum and the residue was purified by silica gel column chromatography (petroleum ether:ethyl acetate=100:1 to 10/1) to afford the title compound (300 mg, 54% yield) as a white solid. ¹H NMR (CD₃OD, 400 MHz) δ [ppm]: 7.53-7.51 (m, 2H), 7.37-7.32 (m, 3H), 6.71 (s, 1H), 6.68 (s, 1H), 5.17 (s, 2H), 2.49 (s, 3H).

### Step F: 2-(2-(Benzyloxy)-6-methyl-4-(trifluoromethoxy)phenyl)-4,4,5,5-tetramethyl-1,3,2-dioxaborolane

To a mixture of nBuLi (0.53 mL, 1.32 mmol, 1.8 eq) and 1-benzyloxy-2-iodo-3-methyl-5-(trifluoromethoxy)benzene (300.0 mg, 0.74 mmol, 1.0 eq) in THF (10 mL) was added dropwise 2-isopropoxy-4,4,5,5-tetramethyl-1,3,2-dioxaborolane (205.14 mg, 1.1 mmol, 1.5 eq) under nitrogen and stirring was continued at −70° C. for 30 mins. The mixture was poured into sat. aq. NH₄Cl solution (20 mL) and extracted with ethyl acetate (100 mL×3). The combined organic layers were washed with brine (20 mL), dried over anhydrous sodium sulfate, filtered, and the filtrate was concentrated under reduced pressure. The residue was purified by silica gel column chromatography (petroleum ether:ethyl acetate=100:1 to 10/1) to give the title compound (110.0 mg, 37% yield) as a white solid. ¹H NMR (CD₃OD, 400 MHz) δ [ppm]: 7.48-7.46 (m, 2H), 7.37-7.32 (m, 3H), 6.88 (s, 1H), 6.74 (s, 1H), 5.02 (s, 2H), 2.34 (s, 3H), 1.27 (s, 12H).

### Step G: 3-Methyl-2-(4,4,5,5-tetramethyl-1,3,2-dioxaborolan-2-yl)-5(trifluoromethoxy)phenol

To a solution of 2-[2-benzyloxy-6-methyl-4-(trifluoromethoxy)phenyl]-4,4,5,5-tetramethyl-1,3,2-dioxaborolane (100 mg, 0.24 mmol, 1.0 eq) in EtOAc (2 mL)/methanol (2 mL) was added Pd/C (10.0 mg, 10%) under nitrogen. The suspension was degassed under vacuum and purged with H₂ several times. The mixture was stirred under H₂ atmosphere at 20° C. for 2 hrs. The suspension was filtered and the filter cake was washed with MeOH (10 mL×5). The combined filtrates were concentrated to dryness to the title compound (60.0 mg, 0.19 mmol, 77% yield) as a yellow oil. ¹H NMR (CD₃OD, 400 MHz) δ [ppm]: 6.54 (s, 1H), 6.48 (s, 1H), 2.43 (s, 3H), 1.38 (s, 12H).

### Step H: 2-[2-[[(3R)-1-Ethyl-3-piperidyl]amino]oxazolo[4,5-b]pyridin-5-yl]-3-methyl-5-(trifluoromethoxy)phenol;2,2,2-trifluoroacetic acid

To a mixture of 3-methyl-2-(4,4,5,5-tetramethyl-1,3,2-dioxaborolan-2-yl)-5-(trifluoromethoxy)phenol (20.0 mg, 0.06 mmol, 1.0 eq), 5-bromo-N-[(3R)-1-ethyl-3-piperidyl]oxazolo[4,5-b]pyridin-2-amine;2,2,2-trifluoroacetic acid (Example 21, step H) (33.1 mg, 0.08 mmol, 1.2 eq) and K₂CO₃ (34.7 mg, 0.25 mmol, 4.0 eq) in 1,4-dioxane (1 mL)/water (0.2 mL) was added Pd(dppf)Cl₂ (2.3 mg, 0.0 mmol, 0.05 eq) under nitrogen atmosphere and stirring was continued at 95° C. for 2 hrs. The mixture was filtered and the filtrate was concentrated in vacuum. The crude was purified by column chromatography (C₁₈, 0.10% TFA in water/MeCN) to afford the title compound (12.3 mg, 35% yield) as a yellow solid. LCMS: m/z 437.1, [M+H]⁺, ESI pos.

### Example 138

### 2-[2-(1-Ethyl-3-piperidyl)oxazolo[4,5-b]pyridin-5-yl]-3-methyl-5-(trifluoromethyl)phenol

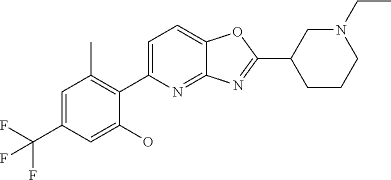

### Step A: 5-Bromo-2-(1-ethylpiperidin-3-yl)oxazolo[4,5-b]pyridine

The mixture of 2-amino-6-bromopyridin-3-ol (100 mg, 0.53 mmol, 1.0 eq) and 1-ethylpiperidine-3-carboxylic acid (83.2 mg, 0.53 mmol, 1.0 eq) in PPA (1.0 mL) was stirred at 150° C. for 4 hrs. After cooling to ambient temperature, the reaction mixture was diluted with ice-water (150 mL), then treated with saturated sodium carbonate to adjusted the pH to about 10 and extracted with ethyl acetate (50 mL×3). The combined organic phases were washed with brine (100 mL), dried over anhydrous sodium sulfate, filtered and the filtrate was concentrated under reduced pressure. The residue was purified by prep-HPLC (Column Waters Xbridge 150×25 mm×5 μm Condition water (ammonia hydroxide v/v)-ACN Begin B 5 End B 35 Gradient Time (min) 9100% B Hold Time(min) 2 FlowRate (ml/min) 25) to afford the title compound (10 mg, 5% yield) as a yellow solid. LCMS: m/z 312.0 [M+2+H]⁺ (Br isotopes), ESI pos.

### Step B: 2-[2-(1-Ethyl-3-piperidyl)oxazolo[4,5-b]pyridin-5-yl]-3-methyl-5-(trifluoromethyl)-phenol

To a solution of 5-bromo-2-(1-ethylpiperidin-3-yl)oxazolo[4,5-b]pyridine (10.0 mg, 0.03 mmol, 1.0 eq) in 1,4-dioxane (1 mL) and water (0.2 mL) was added (2-hydroxy-6-methyl-4-(trifluoromethyl)phenyl)boronic acid (14.2 mg, 0.06 mmol, 2.0 eq), Na₂CO₃ (10.3 mg, 0.1 mmol, 3.0 eq), and Pd(dppf)Cl₂ (4.72 mg, 0.01 mmol, 0.2 eq). The above reaction mixture was stirred at 100° C. for 2 hrs under nitrogen atmosphere. After cooling to ambient temperature, the reaction mixture was filtered and the filtrate was concentrated under reduced pressure. The crude was purified by column chromatography (Cis, 0.1% NH₃—H₂O in water-MeCN) to afford the title compound (7.97 mg, 60% yield) as yellow oil. LCMS: m/z 406.0 [M+H]⁺, ESI pos.

### Example 139a and 139b

### 4-[2-[[(8S,8aS)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8R,8aR)-1,2,3,5,6,7,8,8a-octahydroindolizin-8-yl]amino]oxazolo[4,5-b]pyridin-5-yl]-3-hydroxy-5-methyl-benzonitrile and 4-[2-[[(8R,8aR)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8S,8aS)-1,2,3,5,6,7,8,8a-octahydroindolizin-8-yl]amino]oxazolo[4,5-b]pyridin-5-yl]-3-hydroxy-5-methyl-benzonitrile

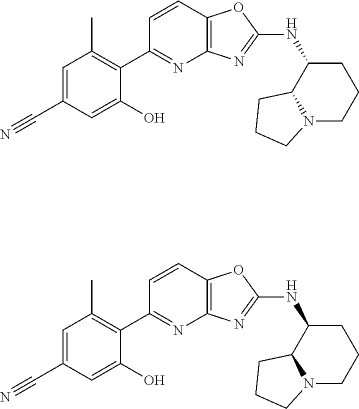

### Step A: N-[rel-(8R,8aR)-1,2,3,5,6,7,8,8a-octahydroindolizin-8-yl]-5-bromo-oxazolo[4,5-b]pyridin-2-amine

To a mixture of rel-(8R,8aR)-1,2,3,5,6,7,8,8a-octahydroindolizin-8-amine;dihydrochloride commercially available (603 mg, 2.83 mmol, 1.1 eq), DIEA (1.66 g, 12.9 mmol, 5.0 eq) in DMF (10 mL) was added 5-bromo-2-chloro-oxazolo[4,5-b]pyridine (600 mg, 2.57 mmol, 1.0 eq) and stirred for 2 hrs at 25° C. The mixture was filtered and the filtrate was concentrated in vacuum. The crude product was purified by reversed phase flash (water (0.1% TFA)-MeCN) to give the title compound (700 mg, 81% yield) as a yellow solid. LCMS: m/z 339.1, [M+H]⁺ ESI pos.

### Step B: 3-Hydroxy-5-methyl-4-[2-[[rel-(8R,8aR)-1,2,3,5,6,7,8,8a-octahydroindolizin-8-yl]amino]oxazolo[4,5-b]pyridin-5-yl]benzonitrile

To a mixture of (4-cyano-2-hydroxy-6-methyl-phenyl)boronic acid (Example 25, step D) (661.3 mg, 3.74 mmol, 1.8 eq), CsF (1.26 g, 8.3 mmol, 4.0 eq), N-[rel-(8R,8aR)-1,2,3,5,6,7,8,8a-octahydroindolizin-8-yl]-5-bromo-oxazolo[4,5-b]pyridin-2-amine (700 mg, 2.08 mmol, 1.0 eq) in 1,4-dioxane (20 mL)/water (4 mL) was added XPhos Pd G3 (175.9 mg, 0.21 mmol, 0.1 eq) and stirred for 2 hrs at 95° C. The mixture was filtered and the filtrate was concentrated in vacuum. The crude product was purified by reversed phase flash (water (0.1% TFA)-MeCN) to give the title compound (400 mg, 49% yield) as yellow solid. LCMS: m/z 390.2 [M+H]⁺ ESI pos.

### Step C: 4-[2-[[(8S,8aS)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8R,8aR)-1,2,3,5,6,7,8,8a-octahydroindolizin-8-yl]amino]oxazolo[4,5-b]pyridin-5-yl]-3-hydroxy-5-methyl-benzonitrile and 4-[2-[[(8R,8aR)-1,2,3,5,6,7,8,8a-Octahydroindolizin-8-yl]amino]oxazolo[4,5-b]pyridin-5-yl]-3-hydroxy-5-methyl-benzonitrile or 4-[2-[[(8S,8aS)-1,2,3,5,6,7,8,8a-octahydroindolizin-8-yl]amino]oxazolo-[4,5-b]pyridin-5-yl]-3-hydroxy-5-methyl-benzonitrile

The title compounds were obtained after chiral separation of aforementioned 3-hydroxy-5-methyl-4-[2-[[rel-(8R,8aR)-1,2,3,5,6,7,8,8a-octahydroindolizin-8-yl]amino]oxazolo[4,5-b]pyridin-5-yl]benzonitrile (400 mg, 1.03 mmol, 1.0 eq) by SFC separation. (Instrument: ACSWH-PREP-SFC-C, Method Column: DAICEL CHIRALPAK IC (250 mm×30 mm×10 μm). Enantiomer 1 (139a): (111.4 mg, 28% yield) as a white solid ¹H NMR (CD₃OD, 400 MHz) δ [ppm]: 7.66 (d, 1H), 7.13 (s, 1H), 7.03 (s, 1H), 6.99 (d, 1H), 3.77-3.67 (m, 1H), 3.15-3.05 (m, 2H), 2.32-2.18 (m, 2H), 2.14-2.12 (m, 3H), 2.10-2.09 (m, 1H), 2.08-2.02 (m, 2H), 1.86-1.66 (m, 5H), 1.45-1.31 (m, 1H). LCMS: m/z 390.4, (M+H)⁺ (ESI+) and Enantiomer 2 (139b) (112.3 mg, 28% yield) as a white solid. ¹H NMR (CD₃OD, 400 MHz) δ [ppm]: 7.67 (d, 1H), 7.13 (s, 1H), 7.03 (s, 1H), 6.98 (d, 1H), 3.77-3.67 (m, 1H), 3.15-3.05 (m, 2H), 2.32-2.18 (m, 2H), 2.14-2.12 (m, 3H), 2.10-2.09 (m, 1H), 2.08-2.02 (m, 2H), 1.86-1.66 (m, 5H), 1.45-1.31 (m, 1H). LCMS: m/z 390.3 [M+H]⁺ ESI pos.

### Example A

A compound of formula I can be used in a manner known per se as the active ingredient for the production of tablets of the following composition:

### Per Tablet

| | | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

### Per Capsule

| | | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula Ic 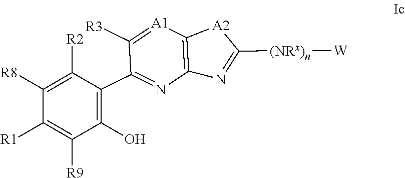
wherein
A¹ is —N— or —CR¹⁰—;
A² is —O—, —NH—, —NCH₃—, or —S—;
R¹ is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 4-to-6-membered cycloalkyl or heterocycle comprising a single O heteroatom;
R² is H, halo, alkyl, cyano, haloalkoxy, haloalkyl, alkoxy, alkoxyalkyl, or hydroxyalkyl, and R³ is H, halo, or alkyl, wherein at least one of R² and R³ is not H;
R⁸ is H or halo;
R¹⁰ is selected from H, alkyl, acetyl, alkylamino, dialkylamino, hydroxyalkyl, hydroxyalkylalkyl, cyano, halo, haloalkyl, alkoxy, haloalkoxy, and NR′R″, wherein R′ and R″ are independently selected from H and alkyl, or R′ and R″ and the N atom to which they are attached form either a 4-to-6 membered N-containing heterocycle optionally substituted with —OH, or a 5-membered lactam ring;
n can be 0 or 1;
R^{X} is H, alkyl, alkoxyalkyl, or hydroxyalkyl;
W is selected from
a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, cyano, haloalkyl, alkylester, alkylsulfon, cycloalkyl, heterocycle comprising a single O heteroatom, cycloalkyl substituted with —OH, cycloalkyl-CH₂— substituted with —OH, a 6-membered heteroaryl substituted with alkoxy, and a 5-membered heteroaryl with 2 N heteroatoms,
a 4-to-6-membered heterocycle-CH₂— comprising up to 2 heteroatoms independently selected from N and O, wherein the maximum number of O heteroatoms is 1, optionally substituted by alkyl,
a 3-to-6 membered cycloalkyl optionally substituted with 1 to 2 substituents independently selected from —OH, alkylamino and dialkylamino,
a 4-membered cycloalkyl-CH₂— optionally substituted with 1 or 2 substituents independently selected from —OH and alkyl, and
a 5-membered heteroaryl-CH₂— comprising 2 N heteroatoms;
and pharmaceutically acceptable salts.

2. A compound according to claim 1, wherein A² is —O— or —NH—.

3. A compound according to claim 1, wherein A² is —O—.

4. A compound according to claim 1, wherein
R is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 5-membered heterocycle comprising a single O heteroatom.

5. A compound according to claim 1, wherein
R¹ is halo, alkyl, haloalkyl, or cyano;
R⁹ is H;
or R¹ and R⁹ form a 5-membered heterocycle comprising a single O heteroatom.

6. A compound according to claim 1, wherein R¹ is halo or cyano and R⁹ is H.

7. A compound according to claim 1, wherein R² is alkyl or alkoxyalkyl and R³ is H.

8. A compound according to claim 1, wherein R⁸ is H.

9. A compound according to claim 1, wherein R¹⁰ is selected from H, alkyl, acetyl, alkylamino, dialkylamino, hydroxyalkyl, hydroxyalkylalkyl, cyano, alkoxy, and NR′R″, wherein R′ and R″ and the N atom to which they are attached form either a 4-to-5-membered N-containing heterocycle optionally substituted with —OH, or a 5-membered lactam ring.

10. A compound according to claim 1, wherein R¹⁰ is selected from H, cyano, and alkoxy.

11. (canceled)

12. A compound according to claim 1, wherein R^{X} is H, alkoxyalkyl, or hydroxyalkyl.

13. (canceled)

14. A compound according to claim 1, wherein W is selected from
a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, haloalkyl, a 4-membered cycloalkyl, a 4-membered heterocycle comprising 1 O heteroatom, a 4-membered cycloalkyl substituted with —OH, and a 4-membered cycloalkyl-CH₂— substituted with —OH,
a 4-to-6-membered heterocycle-CH₂— comprising up to 2 heteroatoms independently selected from N and O, wherein the maximum number of O heteroatoms is 1, optionally substituted by alkyl,
a 3-to-6-membered cycloalkyl optionally substituted with —OH, alkylamino or dialkylamino,
a 4-membered cycloalkyl-CH₂— optionally substituted with 1 or 2 substituents independently selected from —OH and alkyl, and
a 5-membered heteroaryl-CH₂— comprising 2 N heteroatoms.

15. A compound according to claim 1, wherein W is selected from
a 6-to-9-membered heterocycle comprising up to 3 heteroatoms independently selected from N and O, wherein the maximum number of O heteroatoms is 1, optionally substituted by 1 or 2 substituents selected from oxo, —OH, halo, alkyl and hydroxyalkyl,
a 6-membered heterocycle-CH₂— comprising a single N heteroatom, substituted by alkyl, and
a 5-membered cycloalkyl substituted with dialkylamino.

16. A compound according to claim 1, wherein W is a 6-membered heterocycle comprising a single N heteroatom, optionally substituted by either alkyl or oxo, or both alkyl and —OH.

17. A compound according to claim 1, wherein
A¹ is —N— or —CR¹⁰—;
A² is —O— or —NH—;
R¹ is H, halo, alkyl, haloalkyl, haloalkoxy, cyano, alkoxy, cycloalkyl or alkylsulfonyl;
R⁹ is H, alkyl, halo or cyano;
or R¹ and R⁹ form a 5-membered heterocycle comprising a single O heteroatom;
R² is H, halo, alkyl, cyano, haloalkoxy, haloalkyl, alkoxy, alkoxyalkyl, or hydroxyalkyl, and R³ is H, halo, or alkyl, wherein at least one of R² and R³ is not H;
R⁸ is H or halo;
R¹⁰ is selected from H, alkyl, acetylalkylamino, dialkylamino, hydroxyalkyl, hydroxyalkylalkyl, cyano, alkoxy, and NR′R″, wherein R′ and R″ and the N atom to which they are attached form either a 4-to-5-membered N-containing heterocycle optionally substituted with —OH, or a 5-membered lactam ring;
n can be 0 or 1;
R^{X} is H, alkoxyalkyl, or hydroxyalkyl;
W is selected from
a 4-to-10-membered heterocycle comprising up to 3 heteroatoms independently selected from N and O, wherein the maximum number of O heteroatoms is 1, optionally substituted by 1 or 2 substituents selected from oxo, —OH, halo, alkyl, alkoxyalkyl, alkylamino, dialkylaminoalkyl, hydroxyalkyl, haloalkyl, a 4-membered cycloalkyl, a 4-membered heterocycle comprising a single O heteroatom, a 4-membered cycloalkyl substituted with —OH, and a 4-membered cycloalkyl-CH₂— substituted with —OH,
a 4-to-6-membered heterocycle-CH₂— comprising up to 2 heteroatoms independently selected from N and O, wherein the maximum number of O heteroatoms is 1, optionally substituted by alkyl,
a 3-to-6-membered cycloalkyl optionally substituted with —OH, alkylamino or dialkylamino,
a 4-membered cycloalkyl-CH₂— optionally substituted with 1 or 2 substituents independently selected from —OH and alkyl, and
a 5-membered heteroaryl-CH₂— comprising 2 N heteroatoms;
and pharmaceutically acceptable salts.

18. A compound according to claim 1, wherein
A¹ is —N— or —CR¹⁰—;
A² is —O—;
R¹ is halo, alkyl, haloalkyl, or cyano;
R⁹ is H;
or R¹ and R⁹ form a 5-membered heterocycle comprising a single O heteroatom;
R² is alkyl or alkoxyalkyl and R³ is H;
R⁸ is H;
R¹⁰ is selected from H, cyano and alkoxy;
n can be 0 or 1;
R^{X} is H;
W is selected from
a 6-to-9-membered heterocycle comprising up to 3 heteroatoms independently selected from N and O, wherein the maximum number of O heteroatoms is 1, optionally substituted by 1 or 2 substituents selected from oxo, —OH, halo, alkyl and hydroxyalkyl,
a 6-membered heterocycle-CH₂— comprising a single N heteroatom, substituted by alkyl, and
a 5-membered cycloalkyl substituted with dialkylamino;
and pharmaceutically acceptable salts.

19. A compound according to claim 1, wherein
A¹ is —N— or —CR¹⁰—;
A² is —O—;
R¹ is halo or cyano;
R⁹ is H;
R² is alkyl or alkoxyalkyl and R³ is H;
R⁸ is H;
R¹⁰ is H;
n can be 0 or 1;
R^{X} is H;
W is a 6-membered heterocycle comprising a single N heteroatom, optionally substituted by either alkyl or oxo, or both alkyl and —OH;
and pharmaceutically acceptable salts.

20. A compound according to claim 1, selected from
(rac)-2-[2-[(1-Ethyl-3-piperidyl)amino]oxazolo[4,5-b]pyridin-5-yl]-3-methyl-5-(trifluoromethyl)phenol;
(rac)-2-[2-[3-[(Dimethylamino)methyl]morpholin-4-yl]oxazolo[4,5-b]pyridin-5-yl]-3-methyl-5-(trifluoromethyl)phenol;
2-[2-[(3R or 3S)-3-[(Dimethylamino)methyl]morpholin-4-yl]oxazolo[4,5-b]pyridin-5-yl]-3-methyl-5-(trifluoromethyl)phenol;
2-[2-[(3S or 3R)-3-[(Dimethylamino)methyl]morpholin-4-yl]oxazolo[4,5-b]pyridin-5-yl]-3-methyl-5-(trifluoromethyl)phenol;
(rac)-1-[5-[2-Hydroxy-6-methyl-4-(trifluoromethyl)phenyl]oxazolo[4,5-b]pyridin-2-yl]piperidin-3-ol;
(3R or 3S)-1-[5-[2-Hydroxy-6-methyl-4-(trifluoromethyl)phenyl]oxazolo[4,5-b]pyridin-2-yl]piperidin-3-ol;
(3S or 3R)-1-[5-[2-Hydroxy-6-methyl-4-(trifluoromethyl)phenyl]oxazolo[4,5-b]pyridin-2-yl]piperidin-3-ol;
3-Methyl-2-(2-morpholinooxazolo[4,5-b]pyridin-5-yl)-5-(trifluoromethyl)phenol;
(rac)-[2-[2-(Hydroxymethyl)morpholin-4-yl]oxazolo[4,5-b]pyridin-5-yl]-3-methyl-5-(trifluoromethyl)phenol;
3-Methyl-2-(2-piperazin-1-yloxazolo[4,5-b]pyridin-5-yl)-5-(trifluoromethyl)phenol;
3-Methyl-2-[2-[[3-(methylamino)cyclohexyl]amino]oxazolo[4,5-b]pyridin-5-yl]-5-(trifluoromethyl)phenol;
3-Methyl-2-(2-piperazin-1-yl-1H-imidazo[4,5-b]pyridin-5-yl)-5-(trifluoromethyl)phenol;
(rac)-5-(5-(2-Hydroxy-6-methyl-4-(trifluoromethyl)phenyl)oxazolo[4,5-b]pyridin-2-yl) piperidin-2-one;
(rac)-3-Methyl-2-[2-[3-(methylamino)-1-piperidyl]oxazolo[4,5-b]pyridin-5-yl]-5-(trifluoromethyl) phenol;
(rac)-3-Methyl-2-[2-(tetrahydropyran-3-ylamino)oxazolo[4,5-b]pyridin-5-yl]-5-(trifluoromethyl)phenol;
1-[5-[2-Hydroxy-6-methyl-4-(trifluoromethyl)phenyl]oxazolo[4,5-b]pyridin-2-yl]azetidin-3-ol;
2-[2-[(3-Hydroxycyclohexyl)amino]oxazolo[4,5-b]pyridin-5-yl]-3-methyl-5-(trifluoromethyl)phenol;
2-[2-[[(3R)-1-Ethyl-3-piperidyl]amino]oxazolo[4,5-b]pyridin-5-yl]-3-methyl-5-(trifluoromethyl)phenol;
3-Methyl-2-[2-[[(3R)-1-methyl-3-piperidyl]amino]oxazolo[4,5-b]pyridin-5-yl]-5-(trifluoromethyl)phenol;
(rac)-3-Methyl-2-(2-(1-methylpiperidin-2-yl)-3H-imidazo[4,5-b]pyridin-5-yl)-5-(trifluoromethyl)phenol;
(rac)-3-Methyl-2-(2-(pyrrolidin-2-yl)-1H-imidazo[4,5-b]pyridin-5-yl)-5-(trifluoro-methyl)phenol;
(rac)-2-[2-[(1-Ethyl-3-piperidyl)amino]-1H-imidazo[4,5-b]pyridin-5-yl]-3-methyl-5-(trifluoromethyl)phenol;
2-[2-[[(3R or 3S)-1-Ethyl-3-piperidyl]amino]-1H-imidazo[4,5-b]pyridin-5-yl]-3-methyl-5-(trifluoromethyl)phenol;
2-[2-[[(3S or 3R)-1-Ethyl-3-piperidyl]amino]-1H-imidazo[4,5-b]pyridin-5-yl]-3-methyl-5-(trifluoromethyl)phenol;
5Chloro-h2-[2-[[(3R)-1-ethyl-3-piperidyl]amino]oxazolo[4,5-b]pyridin-5-yl]-3-methyl-phenol;
5-Chloro-3-methyl-2-[2-(6-methyl-3,3a,4,5,7,7a-hexahydro-2H-pyrrolo[2,3-c]pyridin-1-yl)oxazolo[4,5-b]pyridin-5-yl]phenol;
2-[2-[(3aS,7aR or 3aR,7aS)-6-Methyl-3,3a,45,7,7a-hexahydro-2H-pyrrolo[2,3-c]pyridin-1-yl]oxazolo[4,5-b]pyridin-5-yl]-5-chloro-3-methyl-phenol;
2-[2-[(3aR,7aS or 3aS,7aR)-6-Methyl-3,3a,4,5,7,7a-hexahydro-2H-pyrrolo[2,3-c]pyridin-1-yl]oxazolo[4,5-b]pyridin-5-yl]-5-chloro-3-methyl-phenol;
5-Chloro-2-[2-[[(3R)-1-ethyl-3-piperidyl]amino]oxazolo[4,5-b]pyridin-5-yl]-6-fluoro-3-methyl-phenol;
5-Chloro-2-[2-[[(3R)-1-ethyl-3-piperidyl]amino]oxazolo[4,5-b]pyridin-5-yl]-4-fluoro-3-methyl-phenol;
5-Chloro-2-[2-[[(3R)-1-ethyl-3-piperidyl]amino]oxazolo[4,5-b]pyridin-5-yl]-3-hydroxy-benzonitrile;
5-Chloro-2-[2-[[(3R)-1-ethyl-3-piperidyl]amino]oxazolo[4,5-b]pyridin-5-yl]-3-(methoxymethyl)phenol;
4-[2-[[(3R)-1-Ethyl-3-piperidyl]amino]oxazolo[4,5-b]pyridin-5-yl]-3-hydroxy-5-methyl-benzonitrile;
3-hydroxy-5-Methyl-4-[2-[[(3R)-1-methyl-3-piperidyl]amino]oxazolo[4,5-b]pyridin-5-yl]benzonitrile;
4-[2-[(3aS,7aR or 3aR,7aS)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[(3aR,7aS or 3aS, 7aR)-6-Methyl-3,3a,4,5,7,7a-hexahydro-2H-pyrrolo[2,3-c]pyridin-1-yl]oxazolo[4,5-b]pyridin-5-yl]-3-hydroxy-5-methyl-benzonitrile;
4-[2-[[(3R,5S)-1-Ethyl-5-hydroxy-3-piperidyl]amino]oxazolo[4,5-b]pyridin-5-yl]-3-hydroxy-5-methyl-benzonitrile;
4-[2-(1,2,3,5,6,7,8,8a-Octahydroindolizin-8-ylamino)oxazolo[4,5-b]pyridin-5-yl]-3-hydroxy-5-methyl-benzonitrile;
3-(Difluoromethyl)-2-[2-[[(3R)-1-methyl-3-piperidyl]amino]oxazolo[4,5-b]pyridin-5-yl]-5-(trifluoromethyl)phenol;
2-[2-[[(3R)-1-Ethyl-3-piperidyl]amino]oxazolo[4,5-b]pyridin-5-yl]-3-methoxy-5-(trifluoromethyl)phenol;
2-[2-[(1-Ethyl-3-piperidyl)amino]oxazolo[4,5-b]pyrazin-5-yl]-3-methyl-5-(trifluoromethyl)phenol;
3-Methyl-2-[7-methyl-2-[[(3R)-1-methyl-3-piperidyl]amino]oxazolo[4,5-b]pyridin-5-yl]-5-(trifluoromethyl)phenol;
5-Methoxy-3-methyl-2-[2-[[(3R)-1-methyl-3-piperidyl]amino]oxazolo[4,5-b]pyridin-5-yl]phenol;
5-[2-[[(3R)-1-Ethyl-3-piperidyl]amino]-6-methyl-oxazolo[4,5-b]pyridin-5-yl]-2,3-dihydrobenzofuran-4-ol;
5-Cyclopropyl-3-methyl-2-[2-[[(3R)-1-methyl-3-piperidyl]amino]oxazolo[4,5-b]pyridin-5-yl]phenol;
2-[2-[(3aS,7aR)-6-Methyl-3,3a,4,5,7,7a-hexahydro-2H-pyrrolo[2,3-c]pyridin-1-yl]oxazolo[4,5-b]pyridin-5-yl]-3-methyl-5-(trifluoromethyl)phenol;
2-[2-[(3aR,7aS)-6-Methyl-3,3a,4,5,7,7a-hexahydro-2H-pyrrolo[2,3-c]pyridin-1-yloxazolo[4,5-b]pyridin-5-yl]-3-methyl-5-(trifluoromethyl)phenol;
(rac)-3-Methyl-2-[2-(tetrahydrofuran-3-ylamino)oxazolo[4,5-b]pyridin-5-yl]-5-(trifluoromethyl)phenol;
5-Chloro-3-methyl-2-[2-[[rac-(8S,8aR)-1,2,3,5,6,7,8,8a-octahydroindolizin-8-yl]amino]oxazolo[4,5-b]pyridin-5-yl]phenol;
2-[2-[[(8S,8aR)-1,2,3,5,6,7,8,8a-Octahydroindolizin-8-yl]amino]oxazolo[4,5-b]pyridin-5-yl]-5-chloro-3-methyl-phenol;
2-[2-[[(8R,8aS)-1,2,3,5,6,7,8,8a-Octahydroindolizin-8-yl]amino]oxazolo[4,5-b]pyridin-5-yl]-5-chloro-3-methyl-phenol;
or;
5-Chloro-3-methyl-2-[2-(6-methyl-3,4a,5,7,8,8a-hexahydro-2H-pyrido[4,3-b][1,4]oxazin-4-yl)oxazolo[4,5-b]pyridin-5-yl]phenol;
2-[2-(2-Azabicyclo[2.1.1]hexan-1-ylmethylamino)oxazolo[4,5-b]pyridin-5-yl]-3-methyl-5-(trifluoromethyl)phenol; dihydrochloride;
5-Chloro-3-methyl-2-[2-[[(3R)-1-methyl-3-piperidyl]amino]oxazolo[4,5-b]pyridin-5-yl]phenol;
2-[2-[[(3R)-1-Ethyl-3-piperidyl]amino]oxazolo[4,5-b]pyridin-5-yl]-5-fluoro-3-methyl-phenol;
5-Chloro-3-methyl-2-[2-[[(3R)-1-propyl-3-piperidyl]amino]oxazolo[4,5-b]pyridin-5-yl]phenol;
5-Chloro-2-[2-[[(3R)-1-cyclobutyl-3-piperidyl]amino]oxazolo[4,5-b]pyridin-5-yl]-3-methyl-phenol;
5-[2-[[(3R)-1-Ethyl-3-piperidyl]amino]oxazolo[4,5-b]pyridin-5-yl]-6-methyl-2,3-dihydrobenzofuran-4-ol;
(3S,5R)-5-[[5-(4-Chloro-2-hydroxy-6-methyl-phenyl)oxazolo[4,5-b]pyridin-2-yl]amino]1-methyl-piperidin-3-ol;
5-Fluoro-3-methyl-2-[2-[[(3R)-1-methyl-3-piperidyl]amino]oxazolo[4,5-b]pyridin-5-yl]phenol;
(3S,5R)-1-Ethyl-5-[[5-[2-hydroxy-6-methyl-4-(trifluoromethyl)phenyl]oxazolo[4,5-b]pyridin-2-yl]amino]piperidin-3-ol;
2-[2-[[(3R)-1-(2-Hydroxyethyl)-3-piperidyl]amino]oxazolo[4,5-b]pyridin-5-yl]-3-methyl-5-(trifluoromethyl)phenol;
2-[2-[[(3R-1-Ethyl-3-piperidyl]amino]oxazolo[4,5-b]pyridin-5-yl]-3-fluoro-5-(trifluoromethyl)phenol;
5-Chloro-2-[2-[(3R,5S)-1-ethyl-5-fluoro-3-piperidyl]amino]oxazolo[4,5-b]pyridin-5-yl]-3-methyl-phenol;
2-[2-[[(1R,2R)-2-(Dimethylamino)cyclopropyl]amino]oxazolo[4,5-b]pyridin-5-yl]-3-methyl-5-(trifluoromethyl)phenol;
3-Hydroxy-4-[2-[[(3R,5S)-5-hydroxy-1-methyl-3-piperidyl]amino]oxazolo[4,5-b]pyridin-5-yl]-5-methyl-benzonitrile;
cis-2-[2-[(3-Hydroxy-3-methyl-cyclobutyl)methylamino]oxazolo[4,5-b]pyridin-5-yl]-3-methyl-5-(trifluoromethyl)phenol;
trans-2-[2-[(3-1-Hydroxy-3-methyl-cyclobutyl)methylamino]oxazolo[4,5-b]pyridin-5-yl-3-methyl-5-(trifluoromethyl)phenol;
5-Chloro-2-[2-[(1-ethylazetidin-3-yl)methylamino]oxazolo[4,5-b]pyridin-5-yl]-3-methyl-phenol;
1-[5-(4-(Chloro-2-hydroxy-6-methyl-phenyl)-2-[[rac-(3R)-1-methyl-3-piperidyl]amino]oxazolo[4,5-b]pyridin-7-yl]pyrrolidin-2-one;
2-[2-[[(3R)-1-(3-Hydroxycyclobutyl)-3-piperidyl]amino]oxazolo[4,5-b]pyridin-5-yl]-3-methyl-5-(trifluoromethyl)phenol;
2-[2-(1,3,3a,4,6,6a-Hexahydrofuro[3,4-c]pyrrol-5-yl)oxazolo[4,5-b]pyridin-5-yl]-3-methyl-5-(trifluoromethyl)phenol;
5-Chloro-3-methyl-2-[2-[[(3R)-1-(oxetan-3-yl)-3-piperidyl]amino]oxazolo[4,5-b]pyridin-5-yl]phenol;
3-Methyl-2-[2-[rel-(4aR,7aR)-3,4a,5,6,7,7a-hexahydro-2H-pyrrolo[3,4-b][1,4]oxazin-4-yl]oxazolo[4,5-b]pyridin-5-yl]-5-(trifluoromethyl)phenol;2,2,2-trifluoroacetic acid;
2-[2-[(4aS,7aS)-3,4a,5,6,7,7a-Hexahydro-2H-pyrrolo[3,4-b]1,4]oxazin-4-yl]oxazolo[4,5-b]pyridin-5-yl]-3-methyl-5-(trifluoromethyl)phenol;
2-[2-[(4aS,7aS)-3,4a,5,6,7,7a-Hexahydro-2H-pyrrolo[3,4-b]1,4]oxazin-4-yl]oxazolo[4,5-b]pyridin-5-yl]-3-methyl-5-(trifluoromethyl)phenol;
N-(1,2,3,5,6,7,8,8a-Octahydroindolizin-6-yl)-5-chloro-oxazolo[4,5-b]pyridin-2-amine;
3-Methyl-2-[2-[[(2S)-1-methylazetidin-2-yl]methylamino]oxazolo[4,5-b]pyridin-5-yl]-5-(trifluoromethyl)phenol;
3-methyl-2-[2-[[(2R)-1-methylazetidin-2-yl]methylamino]oxazolo[4,5-b]pyridin-5-yl]-5-(trifluoromethyl)phenol;
3-Methyl-2-[2-[(1-methylazetidin-2-yl)methylamino]oxazolo[4,5b]pyridin-5-yl]-5-(trifluoromethyl)phenol;
5-Chloro-2-[2-(1H-imidazol-5-ylmethylamino)oxazolo[4,5-b]pyridin-5-yl]-3-methyl-phenol;
(3RS,5R)-5-[[5-(4-Chloro-2-hydroxy-6-methyl-phenyl)oxazolo[4,5-b]pyridin-2-yl]amino]-1-ethyl-piperidin-3-ol;formic acid;
3,6-Dimethyl-2-[2-(3R)-1-methyl-3-piperidyl]amino]oxazolo[4,5-b]pyridin-5-yl]phenol;
(3R,5R)-5-[[5-(4-Chloro-2-hydroxy-6-methyl-phenyl)oxazolo[4,5-b]pyridin-2-yl]amino]-1-ethyl-piperidin-3-ol;formic acid;
4-[2-[[(3R,5R)-1-Ethyl-5-hydroxy-3-piperidyl]amino]oxazolo[4,5-b]pyridin-5-yl-3-hydroxy-5-methyl-benzonitrile;
4-[2-[(3aS,7aR)-6-Ethyl-3,3a,4,5,7,7a-hexahydro-2H-pyrrolo[2,3-c]pyridin-1-yl]oxazolo[4,5-b]pyridin-5-yl-3-hydroxy-5-methyl-benzonitrile;
4-[2-[3aR,7aS)-6-ethyl-3,3a,4,5,7,7a-hexahydro-2H-pyrrolo[2,3-c]pyridin-1-yl]oxazolo[4,5-b]pyridin-5-yl-3-hydroxy-5-methyl-benzonitrile;
or;
5-[5-(4-Chloro-2-hydroxy-6-methyl-phenyl]oxazolo[4,5-b]pyridin-2-yl]piperidin-2-one;
(5S)-5-[5-(4-Chloro-2-hydroxy-6-methyl-phenyl)oxazolo[4,5-b]pyridin-2-yl]piperidin-2-one;
(5R)-5-[5-(4-Chloro-2-hydroxy-6-methyl-phenyl oxazolo[4,5-b]pyridin-2-yl]piperidin-2-one;
5-Chloro-2-[2-[[(3R)-1-(3,3-difluoropropyl)-3-piperidyl]amino]oxazolo[4,5-b]pyridin-5-yl]-3-methyl-phenol;
5-Chloro-3-methyl-2-[2-[[(3R)-1-[(3-hydroxycyclobutyl)methyl]-3-piperidyl]amino]oxazolo[4,5-b]pyridin-5-yl]phenol;
5-Chloro-2-[2-(1H-imidazol-2-ylmethylamino)oxazolo[4,5-b]pyridin-5-yl]-3-methyl-phenol;
2-[2-[3-(Hydroxymethyl)-1-piperidyl]oxazolo[4,5-b]pyridin-5-yl]-3-methyl-5-(trifluoromethyl)phenol;
2-[2-[4-(Hydroxymethyl)-1-piperidyl]oxazolo[4,5-b]pyridin-5-yl]-3-methyl-5-(trifluoromethyl)phenol;
2-[2-(1-Ethyl-1,7-diazaspiro[3.5]nonan-7-yl)oxazolo[4,5-b]pyridin-5-yl]3-methyl-5-(trifluoromethyl)phenol;
3-Hydroxy-5-methyl-4-[2-[rac trans-6-ethyl-2,3,4a,5,7,7a-hexahydropyrrolo[3,4-b][1,4]-oxazin-4-yl]oxazolo[4,5-b]pyridin-5-yl]benzonitrile;
3-Hydroxy-5-methyl-4-[2-(7-methyl-2,3,4,4a,5,6,8,8a-octahydro-1,7-naphthyridin-1-yl)oxazolo[4,5-b]pyridin-5-yl]benzonitrile;
2-Hydroxy-4-methyl-3-[2-[[(3R)-1-methyl-3-piperidyl]amino]oxazolo[4,5-b]pyridin-5-yl]benzonitrile;2,2,2-trifluoroacetic acid;
5-Chloro-3-methyl-2-[2-[(1-methyl-3-piperidyl)methyl]oxazolo[4,5-b]pyridin-5-yl]phenol;
Methyl 4-[(3R)-3-[[5-(4-chloro-2-hydroxy-6-methyl-phenyl)oxazolo[4,5-b]pyridin-2-yl]amino]-1-piperidyl]butanoate;
3-Hydroxy-4-[2-[[(3R)-1-(2-hydroxyethyl-3-piperidyl]amino]oxazolo[4,5-b]pyridin-5-yl]-5-methyl-benzonitrile;
Methyl 3-[(3R)-3-[[5-(4-chloro-2-hydroxy-6-methyl-phenyl)oxazolo[4,5-b]pyridin-2-yl]amino]-1-piperidyl]propanoate;
5-Chloro-2-[2-[[6-hydroxymethyl)-1-methyl-3-piperidyl]amino]oxazolo[4,5-b]pyridin-5-yl]-3-methyl-phenol;2,2,2-trifluoroacetic acid;
5-Chloro-2-[2-[(4-isopropylmorpholin-2-yl)methyl]oxazolo[4,5-b]pyridin-5-yl-3-methyl-phenol;formic acid;
3-Methyl-2-[2-[[(3R)-1-methyl-3-piperidyl]amino]oxazolo[4,5-b]pyridin-5-yl]-5-methylsulfonyl-phenol;2,2,2-trifluoroacetic acid;
5-Chloro-2-[2-[2-methoxyethyl-[(3R)-1-methyl-3-piperidyl]amino]oxazolo[4,5-b]pyridin-5-yl]-3-methyl-phenol;formic acid;
2-[2-[[(3R)-1-Ethylazepan-3-yl]amino]oxazolo[4,5-b]pyridin-5-yl-3-methyl-5-(trifluoromethyl)phenol;
5-Chloro-2-[2-[(3-hydroxycyclobutyl)methylamino]oxazolo[4,5-b]pyridin-5-yl]-3-methyl-phenol;
5-Chloro-2-[2-[[(3R)-1-(2-hydroxyethyl)-3-piperidyl)amino]oxazolo[4,5-b]pyridin-5 yl]-3-methyl-phenol;
5-Chloro-3-(hydroxymethyl)-2-[2-[[(3R)-1-methyl-3-piperidyl]amino]oxazolo[4,5-b]pyridin-5-yl]phenol;
5-Chloro-2-[2-[[(3R)-1-(3-hydroxypropyl)-3-piperidyl]amino]oxazolo[4,5-b]pyridin-5 yl]-3-methyl-phenol;
5-Chloro-2-[2-[[rac-(3R)-1-(2-hydroxypropyl)-3-piperidyl]amino]oxazolo[4,5-b]pyridin-5-yl]-phenol;
5-Chloro-2-[2-[2-hydroxyethyl-[(3R)-1-methyl-3-piperidyl]amino]oxazolo[4,5-b]pyridin-5-yl]-3-methyl-phenol;
5-Chloro-2-[2-(((3S,4R)-1-ethyl-4-fluoro-3-piperidyl]amino]oxazolo[4,5-b]pyridin-5-yl]-3-methyl-phenol;
5-Chloro-2-[2-[[(3R,4S)-1-ethyl-4-fluoro-3-piperidyl]amino]oxazolo[4,5-b]pyridin-5-yl]-3-methyl-phenol;
(3S,5R)-5-[[5-[4-Chloro-2-hydroxy-6-(methoxymethyl)phenyl)oxazolo[4,5-b]pyridin-2-yl]amino]-1-methyl-piperidin-3-ol;
1-[2-[[(3R)-1-Ethyl-3-piperidyl]amino]5-[2-hydroxy-6-methyl-4-(trifluoromethyl)phenyl]oxazolo[4,5-b]pyridin-7-yl]azetidin-3-ol;
1-5-[2-Hydroxy-6-methyl-4-(trifluoromethyl)phenyl]-2-[[(3R)-1-methyl-3-piperidyl]amino]oxazolo[4,5-b]pyridin-7-yl]ethanone;2,2,2-trifluoroacetic acid;
2-[2-[[(3R)-1-Ethyl-3-piperidyl]amino]-7-(methylamino)oxazolo[4,5-b]pyridin-5-yl]-3-methyl-5-(trifluoromethyl)phenol,
2-[2-[[(3R)-1-Ethyl-3-piperidyl]amino]-7-methoxy-oxazolo[4,5-b]pyridin-5-yl]-3-methyl-5-(trifluoromethyl)phenol;
2-[7-(1-Hydroxy-1-methyl-ethyl)-2-[[(3R)-1-methyl-3-piperidyl]amino]oxazolo[4,5-b]pyridin-5-yl]-methyl-5-(trifluoromethyl)phenol;2,2,2-trifluoroacetic acid;
2-[7-(1-Hydroxyethyl)-2-[[(3R)-1-methyl-3-piperidyl]amino]oxazolo[4,5-b]pyridin-5-yl]-3-methyl-5-(trifluoromethyl)phenol;
3-Chloro-4-[2-[[(3R)-1-methyl-3-piperidyl]amino]oxazolo 4,5-b]pyridin-5-yl]-5-hydroxy-benzonitrile;2,2,2-trifluoroacetic acid;
4-[2-[[(1R,2R)-2-(Dimethylamino)cyclopentyl]amino]oxazolo[4,5-b]pyridin-5-yl]-3-hydroxy-5-methyl-benzonitrile;
4-[2-[[(3R)-1-Ethyl-3-piperidyl]amino]oxazolo[4,5-b]pyrazin-5-yl]-3-hydroxy-5-methyl-benzonitrile;2,2,2-trifluoroacetic acid;
4-[2-[[(3R)-1-Ethyl-3-piperidyl]amino]oxazolo[4,5-b]pyridin-5-yl]-3-hydroxy-5-(methoxymethyl)benzonitrile;2,2,2-trifluoroacetic acid;
4-[2-[(3R)-1-Ethyl-3-piperidyl]amino]-7-methoxy-oxazolo[4,5-b]pyridin-5-yl-3-hydroxy-5-methyl-benzonitrile;2,2,2-trifluoroacetic acid;
5-Chloro-2-[2-[[(1R,2R)-2-(dimethylamino)cyclopentyl]amino]oxazolo[4,5-b]pyridin-5-yl]-3-methyl-phenol;
5-Chloro-2-[2-[[(3R)-1-ethyl-3-piperidyl]amino]oxazolo[4,5-b]pyrazin-5-yl]-3-methyl-phenol;22,2,2-trifluoroacetic acid;
5-Chloro-2-[2-[[(3R)-1-ethyl-3-piperidyl]amino]oxazolo[4,5-b]pyridin-5-yl]-3-(trifluoromethoxy)phenol;2,2,2-trifluoroacetic acid;
3-Hydroxy-5-methyl-4-[2-[[(2S)-2-hydroxy-1,2,3,5,6,7,8,8a-octahydroindolizin-8-yl]amino]oxazolo[4,5-b]pyridin-5-yl]benzonitrile;2,2,2-trifluoroacetic acid;
2-[7-(Dimethylamino)-2-[[(3R)-1-ethyl-3-piperidyl]amino]oxazolo[4,5-b]pyridin-5-yl]-3-methyl-5-(trifluoromethyl)phenol;
2-[[(3R)-1-Ethyl-3-piperidyl]amino]-5-[2-hydroxy-6-methyl-4-(trifluoromethyl)phenyl]oxazolo[4,5-b]pyridine-7-carbonitrile;
5-chloro-2-[2-[[(3R)-1-(rac trans-2-hydroxycyclobutyl]methyl]-3-piperidyl]amino]oxazolo[4,5-b]pyridin-5-yl]-3-methyl-phenol;
5-chloro-2-[2-[[(3R)-1-[[rac cis-2-hydroxycyclobutyl]methyl]-3-piperidyl]amino]oxazolo[4,5-b]pyridin-5-yl]-3-methyl-phenol;
5-chloro-2-[2-[[(3R)-1-[[rac trans-2-hydroxycyclobutyl]methyl]-3-piperidyl]amino]oxazolo[4,5-b]pyridin-5-yl]-3-methyl-phenol;
2-[2-[[(3R)-1-Ethyl-3-piperidyl]amino]oxazolo[4,5-b]pyridin-5-yl]-3-hydroxy-benzonitrile;
3-Methyl-2-[2-[(2-methyl-2-azabicyclo[2,2,2]octan-4-yl)amino]oxazolo[4,5-b]pyridin-5-yl]-5-(trifluoromethyl)phenol;
5-(Diflouromethyl)-3-methyl-2-[2-[[(3R)-1-methyl-3-piperidyl]amino]oxazolo[4,5-b]pyridin-5-yl]phenol;
5-(Diflouromethyl)-2-[2-[[(3R)-1-ethyl-3-piperidyl]amino]oxazolo[4,5-b]pyridin-5-yl]-3-methyl-phenol;2,2,2-trifluoroacetic acid;
5-(Difluoromethyl)-2-[2-[[(3R)-1-ethyl-3-piperidyl]amino]oxazolo 4,5-b]pyridin-5-yl-3-methyl-phenol;
2-(2-[[(3R)-1-Ethyl-3-piperidyl]amino]oxazolo[4,5-b]pyridin-5-yl]-3,5-dimethyl-phenol;
3-[[5-(4-Chloro-2-hydroxy-6-methyl-phenyl]oxazolo[4,5-b]pyridin-2-yl]amino]-1-ethyl-piperidin-4-ol;
2-[2-[[(3R)-1-Ethyl-3-piperidyl]amino]oxazolo[4,5-b]pyridin-5-yl]-3-methyl-phenol;
5-Fluoro-2-[[(3R)-1-methyl-3-piperidyl]amino]oxazolo[4,5-b]pyridin-5-yl]-3 (trifluoromethyl)phenol;2,2,2-trifluoroacetic acid;
5-Chloro-2-[6-chloro-2-[[(3R)-1-ethyl-3-piperidyl]amino]oxazolo[4,5-b pyridin-5-yl]phenol;
3,5-Dimethyl-2-[2-[[(3R)-1-methyl-3-piperidyl]amino]oxazolo[4,5-b]pyridin-5-yl]phenol;2,2,2-trifluoroacetic acid;
3-Chloro-2-(2-[[(3R)-1-ethyl-3-piperidyl]amino]oxazolo[4,5-b]pyridin-5-yl]-5-fluoro-phenol;2,2,2-trifluoroacetic acid;
5-(Diflouromethyl)-2-[2-[[(3R)-1-ethyl-3-piperidyl]amino]oxazolo[4,5-b]pyridin-5-yl]-3-methyl-phenol;2,2,2-trifluoroacetic acid;
2-[2-(1-Ethylpyrrolidin-3-yl)amino]oxazolo[4,5-b]pyridin-5-yl]-3-methyl-5-(trifluoromethyl)phenol;
3,5-Dichloro-2-[2-[[(3R)-1-ethyl-3-piperidyl]amino]oxazolo[4,5-b]pyridin-5-yl]phenol;2,2,2-trifluoroacetic acid;
2-[2-[[(3R)-1-Ethyl-3-piperidyl]amino]-6-fluoro-1H-imidazo[4,5-b]pyridin-5-yl]-5-(trifluoromethyl)phenol;
(3S,5R)-5-[[5-[4-Chloro-2-hydroxy-6-(methyl-phenyl)oxazolo[4,5-b]pyridin-2-yl]amino]-1-ethyl-piperidin-3-ol;
3-Methyl-2-[2-[4-(methylamino)tetrahydropyran-4-yl]oxazolo[4,5-b]pyridin-5-yl]-5-(trifluoromethyl)phenol;2,2,2-trifluoroacetic acid;
5-Chloro-2-[2-[[(3R)-1-ethyl-3-piperidyl]amino]-3H-imidazo[4,5-b]pyridin-5-yl]-3-methyl-phenol;
2-[2-[[(3R)-1-Ethyl-3-piperidyl]amino]oxazolo[4,5-b]pyridin-5-yl]-3-methyl-5-(trifluoromethoxy)phenol;2,2,2-trifluoroacetic acid;
2-[2-(1-Ethyl-3-piperidyl)oxazolo[4,5-b]pyridin-5-yl]-3-methyl-5-(trifluoromethyl)phenol;
4-[2-[[(8R,8aR)-1,2,3,5,6,7,8,8a-Octahydroindolizin-8-yl]amino]oxazolo[4,5-b]pyridin-5-yl]-3-hydroxy-5-methyl-benzonitrile;
or 4-[2-[[(8S,8aS)-1,2,3,5,6,7,8,8a-Octahydroindolizin-8-yl]amino]oxazolo[4,5-b]pyridin-5-yl]-3-hydroxy-5-methyl-benzonitrile;
and pharmaceutically acceptable salts thereof.

21-29. (canceled)

30. A process to prepare a compound according to claim 1 comprising reacting a compound of formula X to a compound of formula Ic according to the scheme below, wherein R¹, R², R³, R⁸, R⁹, R^{X}, n, and W are as described above, A¹ is N and A² is O.

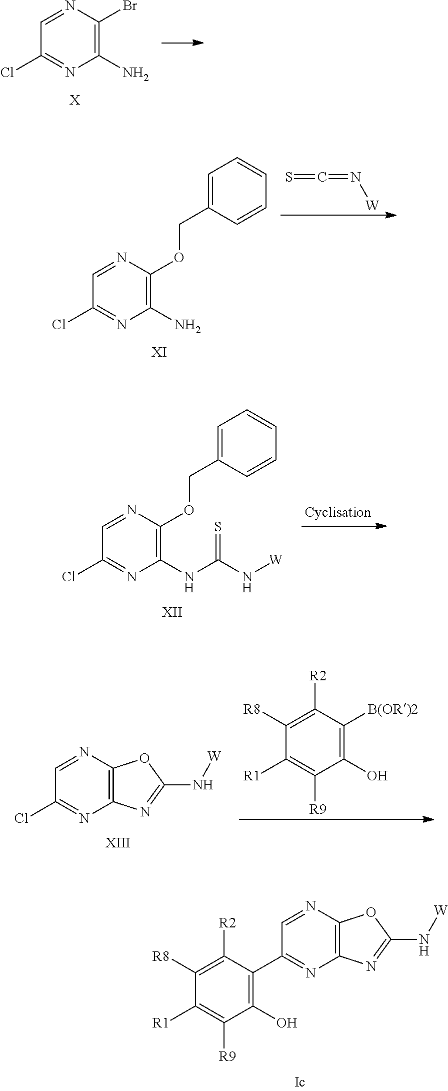

31-33. (canceled)

34. A pharmaceutical composition comprising a compound according to claim 1 and a therapeutically inert carrier.

35-38. (canceled)

39. A method of inhibiting NLRP3, which method comprises administering an effective amount of a compound as claimed in claim 1 to inhibit NLRP3.

40. A method for the treatment or prophylaxis of a disease, disorder or condition, which method comprises administering an effective amount of a compound according to claim 1, wherein the disease, disorder or condition is selected from Asthma or COPD.

41. (canceled)
